(19) United States
(12) Patent Application Publication
(10) Pub. No.: US 2023/0354808 A1
(43) Pub. Date: Nov. 9, 2023

(54) METHOD OF CONTROLLING SOYBEAN RUST FUNGUS RESISTANT TO QOI FUNGICIDE

(71) Applicant: SUMITOMO CHEMICAL COMPANY, LIMITED, Chuo-ku (JP)

(72) Inventors: Yuichi MATSUZAKI, Takarazuka-shi (JP); Takaaki NAKANO, Kasai-shi (JP); Hidekatsu TOBITA, Kasai-shi (JP); Keisuke ARAI, Takarazuka-shi (JP); Nao MAEHATA, Osaka-shi (JP)

(73) Assignee: SUMITOMO CHEMICAL COMPANY, LIMITED, Chuo-ku (JP)

(21) Appl. No.: 17/759,461

(22) PCT Filed: Jan. 29, 2021
(86) PCT No.: PCT/JP2021/003436

§ 371 Date: Jul. 26, 2022

(30) Foreign Application Priority Data
Jan. 31, 2020 (JP) 2020-015187
May 20, 2020 (JP) 2020-088494

(51) Int. Cl. A01N 37/50; A01N 37/38; A01N 43/713; A01N 43/653; A01N 43/88; A01N 43/78; A01N 43/56; A01P 3/00

(52) U.S. Cl. CPC A01N 37/50; A01N 37/38; A01N 43/713; A01N 43/653; A01N 43/88; A01N 43/78; A01N 43/56; A01P 3/00

(57) ABSTRACT

The present invention provides a method for controlling soybean rust fungus having an amino acid replacement of F129L in mitochondrial cytochrome b protein. A compound represented by formula (I) [wherein: Q represents a group represented by Q1 etc. (wherein “•” represents a position bonding to benzene ring); E represents C1-C6 chain hydrocarbon group, etc.; R¹ represents C1-C3 chain hydrocarbon group, etc.; n is 0, 1, 2 or 3; when n is 2 or 3, two or more of R² may be identical to or different from each other; and R² represents C1-C3 chain hydrocarbon group, etc.] can be used for controlling soybean rust fungus having an amino acid replacement of F129L in mitochondrial cytochrome b protein.

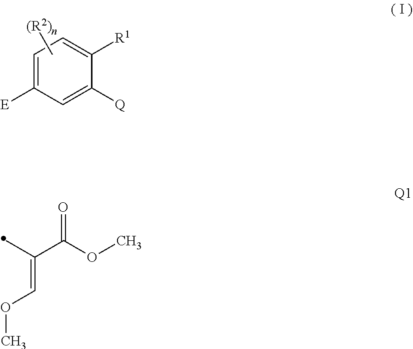

### TECHNICAL FIELD

This application claims priority to and the benefit of Japanese Patent Application Nos. 2020-015187 filed Jan. 31, 2020 and 2020-088494 filed May 20, 2020, the entire contents of which are incorporated herein by reference.

The present invention relates to a method for controlling soybean rust fungus having an amino acid substitution of F129L on mitochondrial cytochrome b protein.

### BACKGROUND ART

The spread of phytopathogenic fungi that shows acquired character being resistant to agricultural fungicides becomes a major problem. Under such circumstances, FRAC (Fungicide Resistance Action Committee) has been established as an organization that provides guidelines for acquiring a resistance to existing agricultural fungicides, and suppressing and delaying the spread of the fungi having the resistance acquired. A variety of information on phytopathogenic fungi that shows a resistance to agricultural fungicides is available on the FRAC-provided website (http://www.frac.info/). It has been known that in the case of a phytopathogenic fungi, the main cause of acquiring a resistance is that a mutation of the phytopathogenic fungal gene encoding the target enzyme of the fungicide causes a partial substitution of amino acids in the target enzyme of the fungicides, which results in reducing the affinity between the fungicides and the target enzyme.

QoI fungicides are named as aliases a strobilurin fungicide, or a methoxyacrylate fungicide because of its characteristic structure. The QoI fungicides are one group of agricultural fungicides that have been widely used to control phytopathogenic fungi including soybean rust fungus. QoI fungicides usually bind to the ubihydroquinone oxidation centers of cytochrome bcl complex (electron transfer complex III) in mitochondria, and suppress a respiration of the phytopathogenic fungi, which results in killing the phytopathogenic fungi or stopping the growth of the same. The above-mentioned oxidation center is located outside the mitochondrial inner membrane (see Non-patent document 1).

It has been revealed by model studies in the laboratory before QoI fungicides were actually used extensively as agricultural fungicides that phytopathogenic fungi are subjected to a selection pressure by QoI fungicide, which results in easily generating the fungi having a resistance to a QoI fungicide that has acquired a gene mutation that causes a specific single amino acid substitution such as G143A in the cytochrome b gene of the target enzyme cytochrome bcl complex (see Non-patent documents 2 to 4).

Soybean rust fungus (scientific name: Phakopsora pachyrhizi) is a phytopathogenic fungus that causes damages to soybeans. Since QoI fungicides have been widely used for controlling soybean rust disease as agricultural fungicides, an emergence of soybean rust fungi showing a resistance to the QoI fungicides has been reported (see Non-patent document 5).

For soybean rust fungus, a strain which has acquired a gene mutation causing a single amino acid substitution of F129L in the same cytochrome b gene becomes a problem as a resistant fungus against QoI fungicide. The efficacy of the QoI fungicides conventionally used against soybean rust fungi, that is, pyribencarb, pyraclostrobin, azoxystrobin, picoxystrpbin, orysastrobin, dimoxystrobin, metominostrobin, and the others, has been reduced to the level of practical problems against the resistant fungi (see Non-patent document 6).

### CITATION LIST

### Non-Patent Document

Non-Patent Document 1: Sauter, “Modern Crop Protection Compounds”, Volume 2, Wiley-VCH Verlag, 2007, p. 457-495: the 13.2 Chapter, Strobilurins and other complex III inhibitors;
Non-Patent Document 2: “Journal of Biological Chemistry”, 1989, Volume 264, no. 24, p. 14543-14548
Non-Patent Document 3: “Genetics”, 1991, Volume 127, p. 335-343
Non-Patent Document 4: “Current Genetics”, 2000, Volume 3, p. 148-155
Non-Patent Document 5: “Pest Management Science”, 2014, Volume 70, no. 3, p. 379-388
Non-Patent Document 6: “Pesq. agropec. bras.” (Brasilia), 2016, Volume 51, no. 5, p. 407-421

### SUMMARY OF THE INVENTION

### Problems to be Solved by Invention

On the basis of these facts, the present invention aims to provide a method for controlling soybean rust fungus having an amino acid substitution of F129L on mitochondrial cytochrome b protein.

### Means to Solve Problems

The present invention is as follows.

[1] A method for controlling a soybean rust fungus having an amino acid substitution of F129L on mitochondrial cytochrome b protein, which comprises applying an effective amount of a compound represented by formula (I):

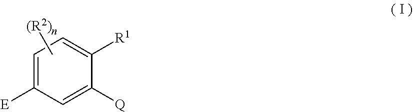

[wherein,
a combination of Q and E represents
   a combination wherein
Q represents a group represented by Q1, or a group represented by Q2,
E represents a C1-C6 chain hydrocarbon group which may be optionally substituted with one or more substituents selected from Group A, a C3-C6 cycloalkyl group which may be optionally substituted with one or more substituents selected from Group B, R⁴R⁵C═C(R⁶)—, R¹⁵—CR¹⁶R³⁶—O—N═C(R⁷)—, R⁸R⁹C═N—O—CH₂—, R¹²O—N═C(R⁷)—C(R⁹)═N—O—CH₂—, R¹²C(O)—C(R⁹)═N—O—CH₂—, R¹²C(═N—O—R⁷)—C(R⁹)═N—O—CH₂—, R³⁴R³⁵C═N—N═C(R¹⁰)—, R¹²R⁹N—N═C(R⁷)—, R¹²SC(R⁷)═N—, R¹²SC(SR⁷)═N—, R¹²N═C(R⁷)—S—CH₂—, R¹²N═C(SR⁷)—S—CH₂—, R¹²O—N═C(R⁷)—S—CH₂—, R¹²O—N═C(SR⁷)—S—CH₂—, R¹²R⁹N—C(S)—O—CH₂—, or R¹⁴O—; or
   a combination wherein
Q represents a group represented by Q3, a group represented by Q4, a group represented by Q5, a group represented by Q6, a group represented by Q7, or a group represented by Q8,
E represents a C1-C6 chain hydrocarbon group which may be optionally substituted with one or more substituents selected from Group A, a C3-C6 cycloalkyl group, a C5-C6 cycloalkenyl group {the C3-C6 cycloalkyl group, and the C5-C6 cycloalkenyl group may be optionally substituted with one or more substituents selected from Group B}, a C6-C10 aryl group, a five- to ten-membered aromatic heterocyclic group {the C6-C10 aryl group and the five- to ten-membered aromatic heterocyclic group may be optionally substituted with one or more substituents selected from Group D}, R⁴R⁵C═C(R⁶)—, R¹³O—N═C(R⁷)—, R⁸R⁹C═N—O—CH₂—, R¹²O—N═C(R⁷)—C(R⁹)═N—O—CH₂—, R¹²C(O)—C(R⁹)═N—O—CH₂—, R¹²C(═N—O—R⁷)—C(R⁹)═N—O—CH₂—, R³⁴R³⁵C═N—N═C(R¹⁰)—, R¹²R⁹N—N═C(R⁷)—, R¹²SC(R⁷)═N—, R¹²SC(SR⁷)═N—, R¹²N═C(R⁷)—S—CH₂—, R¹²N═C(SR⁷)—S—CH₂—, R¹²O—N═C(R⁷)—S—CH₂—, R¹²O—N═C(SR⁷)—S—CH₂—, R¹²R⁹N—C(S)—O—CH₂—, R¹⁹O—, R²⁰—C≡C—, or R¹⁸—N═C(R⁷)—,
each of the group represented by Q1, the group represented by Q2, the group represented by Q3, the group represented by Q4, the group represented by Q5, the group represented by Q6, the group represented by Q7, and the group represented by Q8 represents the following group respectively (where • represents a binding site to a benzene ring),

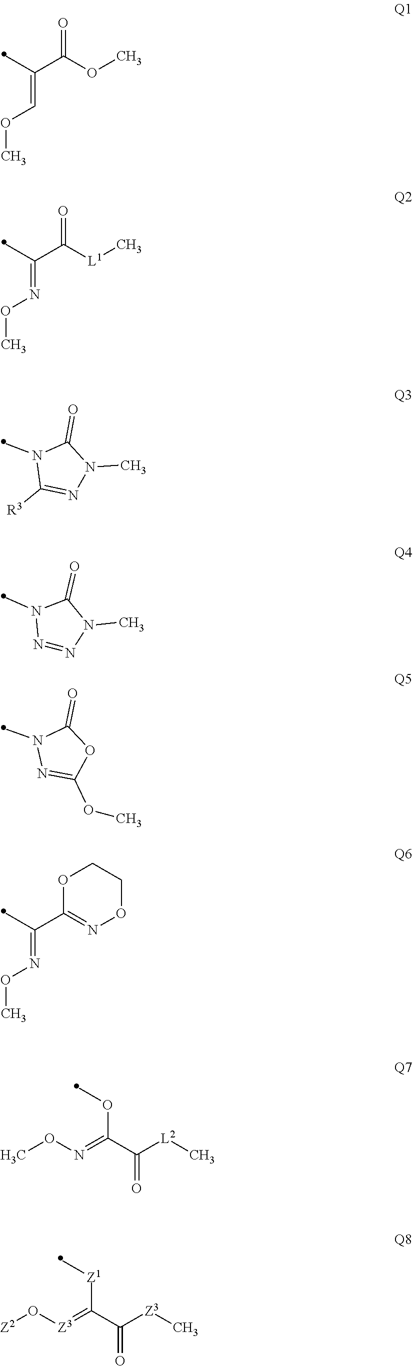

L¹, L² and L³ are identical to or different from each other and represent an oxygen atom or NH,
Z¹ represents a single bond, CH₂, an oxygen atom or NCH₃,
Z² represents a methyl group which is substituted with one or more fluorine atoms,
Z³ represents CH or a nitrogen atom,
R¹ represents a C1-C3 chain hydrocarbon group, a C1-C3 alkoxy group {the C1-C3 chain hydrocarbon group, and the C1-C3 alkoxy group may be optionally substituted with one or more halogen atoms}, a cyclopropyl group, a halogen atom, or a hydrogen atom,
n is 0, 1, 2 or 3,
when n is 2 or 3, a plural of R² may be identical to or different from each other,
R² represents a C1-C3 chain hydrocarbon group which may be optionally substituted with one or more halogen atoms, or a C1-C3 alkoxy group {the C1-C3 chain hydrocarbon group, and the C1-C3 alkoxy group may be optionally substituted with one or more halogen atoms}, a cyclopropyl group, or a halogen atom,
R³ represents a C1-C3 alkoxy group which may be optionally substituted with one or more halogen atoms, or a C1-C3 chain hydrocarbon group which may be optionally substituted with one or more halogen atoms,
R⁴ and R⁶ are identical to or different from each other and represent a C1-C3 chain hydrocarbon group which may be optionally substituted with one or more halogen atoms, a cyclopropyl group, a halogen atom, a cyano group, or a hydrogen atom,
R⁵ represents a C1-C6 chain hydrocarbon group which may be optionally substituted with one or more substituents selected from Group A, a C3-C6 cycloalkyl group which may be optionally substituted with one or more substituents selected from Group B, a C6-C10 aryl group, a five- to ten-membered aromatic heterocyclic group {the C6-C10 aryl group and the five- to ten-membered aromatic heterocyclic group may be optionally substituted with one or more substituents selected from Group D}, a halogen atom, a cyano group, or a hydrogen atom,
R⁸, R¹², R¹³, R¹⁵, R¹⁸, R²⁰, and R³⁴ are identical to or different from each other and represent a C1-C6 chain hydrocarbon group which may be optionally substituted with one or more substituents selected from Group A, a C3-C10 alicyclic hydrocarbon group which may be optionally substituted with one or more substituents selected from Group E, a C6-C10 aryl group, or a five- to ten-membered aromatic heterocyclic group {the C6-C10 aryl group and the five- to ten-membered aromatic heterocyclic group may be optionally substituted with one or more substituents selected from Group D},
R⁷, R⁹, R¹⁰ and R³⁵ are identical to or different from each other and represent a C1-C3 chain hydrocarbon group which may be optionally substituted with one or more halogen atoms, a cyclopropyl group, or a hydrogen atom,
R⁴ and R⁵ may combine together with a carbon atom to which they are attached to form a C3-C10 alicyclic hydrocarbon group or a three- to ten-membered non-aromatic heterocyclic group {the C3-C10 alicyclic hydrocarbon group and the three- to ten-membered non-aromatic heterocyclic group may be optionally substituted with one or more substituents selected from Group E},
R⁸ and R⁹ may combine together with a carbon atom to which they are attached to form a C3-C10 alicyclic hydrocarbon group or a three- to ten-membered non-aromatic heterocyclic group {the C3-C10 alicyclic hydrocarbon group and the three- to ten-membered non-aromatic heterocyclic group may be optionally substituted with one or more substituents selected from Group E},
R³⁴ and R³⁵ may combine together with a carbon atom to which they are attached to form a C3-C10 alicyclic hydrocarbon group or a three- to ten-membered non-aromatic heterocyclic group {the C3-C10 alicyclic hydrocarbon group and the three- to ten-membered non-aromatic heterocyclic group may be optionally substituted with one or more substituents selected from Group E},
R¹⁴ represents a C6-C10 aryl group, a five- to ten-membered aromatic heterocyclic group {the C6-C10 aryl group and the five- to ten-membered aromatic heterocyclic group may be optionally substituted with one or more substituents selected from Group D}, R¹⁷R⁷NC(S)—, R¹⁷O—C(O)—, or R¹⁷C(O)—,
R¹⁵ represents a C6-C10 aryl group, or a five- to ten-membered aromatic heterocyclic group {the C6-C10 aryl group and the five- to ten-membered aromatic heterocyclic group may be optionally substituted with one or more substituents selected from Group C},
R¹⁶ represents a C1-C3 chain hydrocarbon group which may be optionally substituted with one or more halogen atoms, or a cyclopropyl group,
R¹⁷ represents a C1-C6 chain hydrocarbon group which may be optionally substituted with one or more substituents selected from Group A, a C3-C6 cycloalkyl group which may be optionally substituted with one or more substituents selected from Group B, a C6-C10 aryl group, or a five- to ten-membered aromatic heterocyclic group {the C6-C10 aryl group and the five- to ten-membered aromatic heterocyclic group may be optionally substituted with one or more substituents selected from Group C},
R¹⁹ represents a C6-C10 aryl group, a five- to ten-membered aromatic heterocyclic group {the C6-C10 aryl group and the five- to ten-membered aromatic heterocyclic group may be optionally substituted with one or more substituents selected from Group D}, a methyl group which is substituted with one or more substituents selected from Group F, a C2-C6 chain hydrocarbon group which may be optionally substituted with one or more substituents selected from Group F, a C3-C4 cycloalkyl group which may be optionally substituted with one or more substituents selected from Group B, R¹⁷R⁷NC(S)—, R¹⁷O—C(O)—, R¹⁷C(O)—, or R¹⁷R⁷NC(O)—,
R³⁶ represents a C1-C3 chain hydrocarbon group which may be optionally substituted with one or more halogen atoms, a cyclopropyl group, or a hydrogen atom,
Group A is a group consisting of a C3-C6 cycloalkyl group {the C3-C6 cycloalkyl group may be optionally substituted with one or more substituents selected from Group B}, a C1-C4 alkoxy group, a C1-C4 alkylthio group {the C1-C4 alkoxy group and the C1-C4 alkylthio group may be optionally substituted with one or more substituents selected from Group F}, a halogen atom, a cyano group, a nitro group, a hydroxy group, an oxo group, a thioxo group, a phenoxy group, a C6-C10 aryl group, and a five- to ten-membered aromatic heterocyclic group {the phenoxy group, the C6-C10 aryl group and the five- to ten-membered aromatic heterocyclic group may be optionally substituted with one or more substituents selected from Group D},
Group B is a group consisting of a C1-C3 chain hydrocarbon group which may be optionally substituted with one or more halogen atoms, a halogen atom, and a cyano group.
Group C is a group consisting of a C1-C6 chain hydrocarbon group, a C1-C6 alkoxy group, a C1-C6 alkylthio group {the C1-C6 chain hydrocarbon group, the C1-C6 alkoxy group and the C1-C6 alkylthio group may be optionally substituted with one or more substituents selected from Group F}, a C3-C6 cycloalkyl group {the C3-C6 cycloalkyl group may be optionally substituted with one or more substituents selected from Group B}, a halogen atom, a cyano group, a nitro group, and a hydroxy group,
Group D is a group consisting of a C1-C6 chain hydrocarbon group which may be optionally substituted with one or more substituents selected from Group F, a C3-C6 cycloalkyl group which may be optionally substituted with one or more substituents selected from Group B, a C1-C6 alkylthio group which may be optionally substituted with one or more substituents selected from Group F, OR¹¹, C(O)R¹¹, C(O)OR¹¹, NR¹¹R⁷, C(R⁷)═N—OR¹¹, a phenyl group, a naphthyl group, a five- to six-membered aromatic heterocyclic group {the phenyl group, the naphthyl group, and the five- to six-membered aromatic heterocyclic group may be optionally substituted with one or more substituents selected from Group C}, a halogen atom, a cyano group, and a nitro group,
R¹¹ represents a C1-C6 chain hydrocarbon group which may be optionally substituted with one or more substituents selected from Group A, a C3-C6 cycloalkyl group which may be optionally substituted with one or more substituents selected from Group B, a phenyl group, a naphthyl group, a five- to six-membered aromatic heterocyclic group {the phenyl group, the naphthyl group, and the five- to six-membered aromatic heterocyclic group may be optionally substituted with one or more substituents selected from Group C}, or a hydrogen atom,
Group E is a group consisting of an oxo group, a thioxo group, a C1-C3 chain hydrocarbon group, a C1-C3 alkoxy group {the C1-C3 chain hydrocarbon group and the C1-C3 alkoxy group may be optionally substituted with one or more halogen atoms}, a halogen atom, and a cyano group,
Group F is a group consisting of a C3-C4 cycloalkyl group, a halogen atom, and a C1-C3 alkoxy group](hereinafter, referred to as “Present compound N”), or its N oxide or an agriculturally acceptable salt thereof (hereinafter, Compound represented by formula (I), or its N oxide or agriculturally acceptable salts are referred to as “Present compound”).

[2] The method according to [1] wherein the compound represented by formula (I), or its N oxide or an agriculturally acceptable salt thereof represents a compound wherein
Q represents a group represented by Q1, a group represented by Q2, a group represented by Q3, a group represented by Q4, a group represented by Q5, a group represented by Q6, a group represented by Q7, or a group represented by Q8,
E represents R⁴R⁵C═C(R⁶)—, R¹⁵—CR¹⁶R³⁶—O—N═C(R⁷)—, R⁸R⁹C═N—O—CH₂—, R³⁴R³⁵C═N—N═C(R¹⁰)—, or R¹⁴O—,
R⁴ and R⁶ are identical to or different from each other and represent a C1-C3 alkyl group, a halogen atom, or a hydrogen atom,
R⁵ represents a C1-C6 chain hydrocarbon group which may be optionally substituted with one or more substituents selected from Group J, a C3-C6 cycloalkyl group which may be optionally substituted with one or more halogen atoms, or a phenyl group which may be optionally substituted with one or more substituents selected from Group G,
R⁷, R⁹ and R³⁵ are identical to or different from each other and represent a C1-C3 alkyl group, or a hydrogen atom,
R⁸ and R³⁴ are identical to or different from each other and represent a phenyl group, an indanyl group, a tetrahydronaphthyl group, or a five- to six-aromatic heterocyclic group {the phenyl group, the indanyl group, the tetrahydronaphthyl group, and the five- to six-aromatic heterocyclic group may be optionally substituted with one or more substituents selected from Group G},
R⁴ and R⁵ may combine together with a carbon atom to which they are attached to form a C3-C10 alicyclic hydrocarbon group, or a three- to ten-membered non-aromatic heterocyclic group {the C3-C10 alicyclic hydrocarbon group and the three- to ten-membered non-aromatic heterocyclic group may be optionally substituted with one or more substituents selected from Group E},
R⁸ and R⁹ may combine together with a carbon atom to which they are attached to form a C3-C10 alicyclic hydrocarbon group or a three- to ten-membered non-aromatic heterocyclic group {the C3-C10 alicyclic hydrocarbon group and the three- to ten-membered non-aromatic heterocyclic group may be optionally substituted with one or more substituents selected from Group E},
R³⁴ and R³⁵ may combine together with a carbon atom to which they are attached to form a C3-C10 alicyclic hydrocarbon group or a three- to ten-membered non-aromatic heterocyclic group {the C3-C10 alicyclic hydrocarbon group and the three- to ten-membered non-aromatic heterocyclic group may be optionally substituted with one or more substituents selected from Group E},
R¹⁴ and R¹⁵ are identical to or different from each other and represent a phenyl group, or a five- to six-membered aromatic heterocyclic group {the a phenyl group and the five- to six-membered aromatic heterocyclic group may be optionally substituted with one or more substituents selected from Group G},
R¹⁶ represents a C1-C3 alkyl group, and
R³⁶ represents a hydrogen atom,
Group G is a group consisting of a C1-C3 chain hydrocarbon group, a C1-C3 alkoxy group {the C1-C3 chain hydrocarbon group and the C1-C3 alkoxy group may be optionally substituted with one or more halogen atoms}, a halogen atom, and a cyano group,
Group J is a group consisting of a halogen atom and a cyclopropyl group,
   or its N oxide or agriculturally acceptable salt thereof.

[3] The method according to [2] wherein the compound represented by formula (I), or its N oxide or an agriculturally acceptable salt thereof represents a compound wherein
E represents R¹⁵—CR¹⁶R³⁶—O—N═C(R⁷)—, R⁸R⁹C═N—O—CH₂—, or R¹⁴O—,
R⁷ and R⁸ are identical to or different from each other and represent a C1-C3 alkyl group or a hydrogen atom,
R⁸, R¹⁴ and R¹⁵ are identical to or different from each other and represent a phenyl group which may be optionally substituted with one or more substituents selected from Group G,
R¹⁶ represents a C1-C3 alkyl group, and
R³⁶ represents a hydrogen atom,
   or its N oxide or agriculturally acceptable salt thereof.

[4] The method according to [1] wherein the compound respected by formula (I) is a compound wherein
E represents R¹⁴O—,
R¹⁴ represents a thiazolyl group which may be optionally substituted with one or more substituents selected from Group H, or a thienyl group which may be optionally substituted with one or more halogen atoms,
Group H is a group consisting of a C1-C3 chain hydrocarbon group which may be optionally substituted with one or more substituents selected from Group F, a C3-C6 cycloalkyl group, a phenyl group {the C3-C6 cycloalkyl group and the phenyl group may be optionally substituted with one or more halogen atoms}, and a halogen atom.

[5] The method according to any one of [1] to [4] wherein the compound represented by formula (I), or its N oxide or an agriculturally acceptable salt thereof represents a compound wherein
Q represents a group represented by Q1 or a group represented by Q2,
R¹ represents a methyl group, or a halogen atom, and
n is 0,
   or its N oxide or agriculturally acceptable salt thereof.

[6] The method according to any one of [1] to [4] wherein the compound represented by formula (I), or its N oxide or an agriculturally acceptable salt thereof represents a compound wherein
Q represents a group represented by Q1,
R¹ represents a methyl group, and
n is 0,
   or its N oxide or agriculturally acceptable salt thereof.

[7] Use of the compound represented by formula (I) according to any one of [1] to [6], or its N oxide or an agriculturally acceptable salt thereof, for controlling soybean rust fungus having an amino acid substitution of F129L on mitochondrial cytochrome b protein.

[8] A compound represented by formula (II):

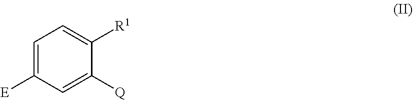

[wherein
R¹ represents a methyl group,
Q represents a group represented by Q1 or a group represented by Q2 (where • represents a binding site to a benzene ring),

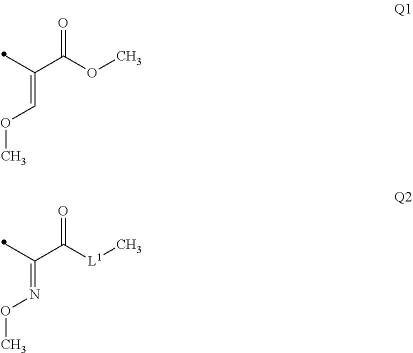

L¹ represents an oxygen atom or NH,
E represents R³⁴R³⁵C═N—N═C(R¹⁰)—, R⁸R⁹C═N—O—CH₂—, R¹⁵—CH(R¹⁶)—O—N═C(R⁷)—, or R¹⁹O—,
R³⁴ represents a C1-C6 chain hydrocarbon group which may be optionally substituted with one or more substituents selected from Group J, a C3-C10 alicyclic hydrocarbon group which may be optionally substituted with one or more substituents selected from Group E, a phenyl group, or a five- to six-membered aromatic heterocyclic group {the phenyl group and the five- to six-membered aromatic heterocyclic group may be optionally substituted with one or more substituents selected from Group G},
R⁹ and R³⁵ are identical to or different from each other and represent a C1-C3 alkyl group, or a hydrogen atom,
R⁷, R¹⁰ and R¹⁶ are identical to or different from each other and represent a C1-C3 alkyl group,
R⁸ represents an indanyl group, a tetrahydronaphthyl group, a dihydrobenzofuranyl group, or a dihydrobenzopyranyl group {the indanyl group, the tetrahydronaphthyl group, the dihydrobenzofuranyl group, and the dihydrobenzopyranyl group may be optionally substituted with one or more substituents selected from Group G},
R⁸ and R⁹ may combine together with a carbon atom to which they are attached to form a C3-C10 alicyclic hydrocarbon group or a three- to ten-membered non-aromatic heterocyclic group {the C3-C10 alicyclic hydrocarbon group and the three- to ten-membered non-aromatic heterocyclic group may be optionally substituted with one or more substituents selected from Group E},
R³⁴ and R³⁵ may combine together with a carbon atom to which they are attached to form a C3-C10 alicyclic hydrocarbon group or a three- to ten-membered non-aromatic heterocyclic group {the C3-C10 alicyclic hydrocarbon group and the three- to ten-membered non-aromatic heterocyclic group may be optionally substituted with one or more substituents selected from Group E},
R¹⁵ represents a phenyl group, or a five- to six-membered aromatic heterocyclic group {the phenyl group and the five- to six-membered aromatic heterocyclic group may be optionally substituted with one or more substituents selected from Group G},
R¹⁹ represents a phenyl group which is substituted at its 2-position with substituent(s) selected from Group L, a phenyl group which is substituted at its 3-position with substituent(s) selected from Group M, a phenyl group which is substituted at its 4-position with substituent(s) selected from Group N, a phenyl group which is substituted with two or more substituents selected from Group P (where the two or more substituents selected from Group P may be identical to or different from each other), a pyridyl group which may be optionally substituted with substituent(s) selected from Group P, a pyrazolyl group which may be optionally substituted with substituent(s) selected from Group G, or a pyrimidinyl group which may be optionally substituted with substituent(s) selected from Group Q,
Group E is a group consisting of an oxo group, a thioxo group, a C1-C3 chain hydrocarbon group, a C1-C3 alkoxy group {the C1-C3 chain hydrocarbon group and the C1-C3 alkoxy group may be optionally substituted with one or more halogen atoms}, a halogen atom, and a cyano group,
Group G is a group consisting of a C1-C3 chain hydrocarbon group, a C1-C3 alkoxy group {the C1-C3 chain hydrocarbon group and the C1-C3 alkoxy group may be optionally substituted with one or more halogen atoms}, a halogen atom, and a cyano group,
Group J is a group consisting of a halogen atom and a cyclopropyl group,
Group L is a group consisting of a C2-C4 chain hydrocarbon group, a C1-C4 alkoxy group {the C2-C4 chain hydrocarbon group and the C1-C4 alkoxy group may be optionally substituted with one or more halogen atoms}, a cyclopropyl group, a halogen atom, and a cyano group,
Group M is a group consisting of a C2-C3 chain hydrocarbon group, a C2-C3 alkoxy group {the C2-C3 chain hydrocarbon group and the C2-C3 alkoxy group may be optionally substituted with one or more halogen atoms}, a trifluoromethyl group, a cyclopropyl group, and a chlorine atom,
Group N is a group consisting of a C1-C3 alkyl group which is substituted with one or more fluorine atoms, a methoxy group, a cyclopropyl group, a halogen atom, and a cyano group,
Group P is a group consisting of a C2-C3 chain hydrocarbon group, a C1-C3 alkoxy group {the C2-C3 chain hydrocarbon group and the C1-C3 alkoxy group may be optionally substituted with one or more halogen atoms}, a halogen atom, and a cyano group,
Group Q is a group consisting of a C1-C3 chain hydrocarbon group which may be optionally substituted with one or more halogen atoms, a C1-C3 alkoxy group, a halogen atom, and a cyano group]
   (hereinafter, referred to as “Present compound C”), or its N oxide or agriculturally acceptable salt thereof.

[9] The compound according to [8] wherein
E represents R¹⁹O—,
R¹⁹ represents a phenyl group which is substituted at its 2-position with substituent(s) selected from Group V, a phenyl group which is substituted at its 4-position with substituent(s) selected from Group V, a 3-chlorophenyl group, a phenyl group which is substituted with two or more halogen atoms, or a pyridyl group which may be optionally substituted with one or more halogen atoms,
Group V is a group consisting of a halogen atom and a methoxy group,
   or its N oxide or agriculturally acceptable salt thereof.

[10] An agricultural composition which comprises the compound according to [8] or [9] or its N-oxide compound or an agriculturally acceptable salt thereof and an inert carrier.

[11] The agricultural composition according to [10] which comprises one or more ingredients selected from the group consisting of the following Groups (a), (b), (c) and (d):
Group (a): a group consisting of insecticidal ingredients, miticidal ingredients, and nematicidal ingredients;
Group (b): fungicidal ingredients:
Group (c): plant growth modulating ingredients; and
Group (d): repellent ingredients.

[12] A method for controlling a soybean rust fungus having an amino acid substitution of F129L on mitochondrial cytochrome b protein, which comprises applying an effective amount of a compound represented by formula (III):

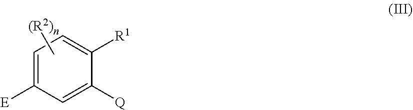

[wherein
E³ represents R³⁹OCH₂—, R⁴⁰CH₂O—, R⁴¹S—, R⁴²SCH₂—, R⁴³CH₂S—, or R⁴⁶O—,
Q represents a group represented by Q1, a group represented by Q2, a group represented by Q3, a group represented by Q4, a group represented by Q5, a group represented by Q6, a group represented by Q7, or a group represented by Q8,
each of the group represented by Q1, the group represented by Q2, the group represented by Q3, the group represented by Q4, the group represented by Q5, the group represented by Q6, the group represented by Q7, and the group represented by Q8 represents the following group respectively (where • represents a binding site to a benzene ring),

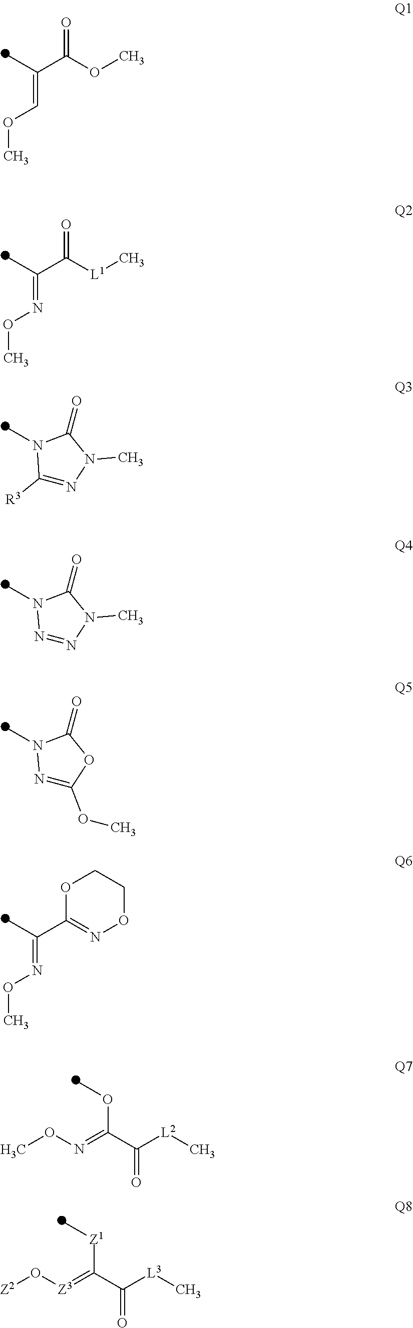

L¹, L² and L³ are identical to or different from each other and represent an oxygen atom, or NH,
Z¹ represents a single bond, CH₂, an oxygen atom or NCH₃,
Z² represents a methyl group which is substituted with one or more fluorine atoms,
Z³ represents CH or a nitrogen atom,
R¹ represents a C1-C3 chain hydrocarbon group, a C1-C3 alkoxy group {the C1-C3 chain hydrocarbon group, and the C1-C3 alkoxy group may be optionally substituted with one or more halogen atoms}, a cyclopropyl group, a halogen atom, or a hydrogen atom,
n is 0, 1, 2, or 3,
when n is 2 or 3, a plural of R² may be identical to or different from each other,
R² represents a C1-C3 chain hydrocarbon group, a C1-C3 alkoxy group {the C1-C3 chain hydrocarbon group, and the C1-C3 alkoxy group may be optionally substituted with one or more halogen atoms}, a cyclopropyl group, or a halogen atom,
R³⁹ represents a five- to ten-membered aromatic heterocyclic group which may be optionally substituted with one or more substituents selected from Group R,
R⁴⁰ represents a C6-C10 aryl group, or a five- to ten-membered aromatic heterocyclic group {the C6-C10 aryl group, and the five- to ten-membered aromatic heterocyclic group may be optionally substituted with one or more substituents selected from Group R},
R⁴¹ represents a C1-C6 chain hydrocarbon group which may be optionally substituted with one or more substituents selected from Group S, a C6-C10 aryl group, a five- to ten-membered aromatic heterocyclic group {the C6-C10 aryl group, and the five- to ten-membered aromatic heterocyclic group may be optionally substituted with one or more substituents selected from Group R}, or a C3-C6 cycloalkyl group which may be optionally substituted with one or more substituents selected from Group T,
R⁴² represents a C6-C10 aryl group, or a five- to ten-membered aromatic heterocyclic group {the C6-C10 aryl group, and the five- to ten-membered aromatic heterocyclic group may be optionally substituted with one or more substituents selected from Group R},
R⁴³ represents a C6-C10 aryl group, or a five- to ten-membered aromatic heterocyclic group {the C6-C10 aryl group, and the five- to ten-membered aromatic heterocyclic group may be optionally substituted with one or more substituents selected from Group R},
R⁴⁶ represents a C5-C6 cycloalkyl group which may be optionally substituted with one or more substituents selected from Group T,
Group R is a group consisting of a C1-C6 chain hydrocarbon group, a C1-C6 alkoxy group, a C1-C6 alkylthio group {the C1-C6 chain hydrocarbon group, the C1-C6 alkoxy group, and the C1-C6 alkylthio group may be optionally substituted with one or more substituents selected from Group S}, a C3-C6 cycloalkyl group which may be optionally substituted with one or more substituents selected from Group T, a phenyl group, a five- to six-membered aromatic heterocyclic group {the phenyl group, and the five- to six-membered aromatic heterocyclic group may be optionally substituted with one or more substituents selected from Group U}, a halogen atom, a cyano group, and a nitro group,
Group S is a group consisting of a C3-C4 cycloalkyl group, a halogen atom, and a C1-C3 alkoxy group,
Group T is a group consisting of a C1-C3 chain hydrocarbon group which may be optionally substituted with one or more halogen atoms, a halogen atom, and a cyano group,
Group U is a group consisting of a C1-C6 chain hydrocarbon group, a C1-C6 alkoxy group, a C1-C6 alkylthio group {the C1-C6 chain hydrocarbon group, the C1-C6 alkoxy group, and the C1-C6 alkylthio group may be optionally substituted with one or more substituents selected from Group S}, a C3-C6 cycloalkyl group which may be optionally substituted with one or more substituents selected from Group T, a halogen atom, a cyano group, and a nitro group.](hereinafter, referred to as “Present compound D”), or its N oxide or agriculturally acceptable salt thereof.

[13] The method according to [12] wherein the compound represented by formula (III) is a compound wherein
E³ represents R³⁹OCH₂—, R⁴⁰CH₂O—, R⁴¹S—, R⁴²SCH₂—, or R⁴³CH₂S—,
R⁴¹ represents a C1-C6 chain hydrocarbon group which may be optionally substituted with one or more substituents selected from Group S, a C6-C10 aryl group, or a five- to ten-membered aromatic heterocyclic group {the C6-C10 aryl group, and the five- to ten-membered aromatic heterocyclic group may be optionally substituted with one or more substituents selected from Group R.}.

[14] Use of the compound represented by formula (III) according to [12] or [13], or its N oxide or an agriculturally acceptable salt thereof, for controlling soybean rust fungus having an amino acid substitution of F129L on mitochondrial cytochrome b protein.

[15] A composition for controlling a plant disease, which comprises a compound represented by the following formula (IV) (hereinafter, referred to as “Present compound A”) and one or more of compounds selected from Group (B) (hereinafter, referred to as “Present compound B”) (hereinafter, a composition is referred to as “Present composition of the present invention”),
a compound represented by formula (IV):

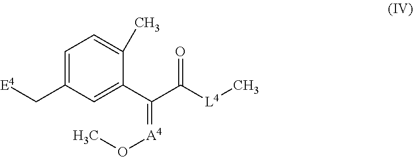

[wherein
E⁴ represents a phenyl group, a 2-methylphenyl group, a 3-methylphenyl group, a 4-methylphenyl group, a 3-fluorophenyl group, a 3-methoxyphenyl group, a 3-(trifluoromethoxy)phenyl group, a 3-phenoxyphenyl group, a 3-thienyl group, a 2-thiazolyl group, a 4-(trifluoromethyl)thizol-2-yl group, a 5-chlorothiazol-2-yl group, a 5-bromothiazol-2-yl group, a 5-(trifluoromethyl)thiazol-2-yl group, a 5-cyclopropylthiazol-2-yl group, a 5-ethynylthiazol-2-yl group, or a (5-cyclopropylethynyl)thiazol-2-yl group,
a combination of A⁴ and L⁴ represents a combination wherein
A⁴ represents CH, and L⁴ represents an oxygen atom; or
   a combination wherein
A⁴ represents a nitrogen atom, and L⁴ represents NH.] Group (B) is a group consisting of the following sub-groups (B-1), (B-2), (B-3) and (B-4),
Sub-group (B-1) is a mitochondrial electron transport chain complex III inhibitor, which consists of coumoxystrobin, enoxastrobin, flufenoxystrobin, picoxystrobin, pyraoxystrobin, pyraclostrobin, mandestrobin, pyrametostrobin, triclopyricarb, trifloxystrobin, dimoxystrobin, fenaminstrobin, metominostrobin, orysastrobin, fluoxastrobin, fenamidone, pyribencarb, metyltetraprole, cyazofamid, amisulbrom, fenpicoxamid, and florylpicoxamid,
Sub-group (B-2) is a mitochondrial electron transport chain complex II inhibitor, which consists of bixafen, fluxapyroxad, benzovindiflupyr, fluindapyr, pydiflumetofen, 3-(difluoromethyl)-N-(2,3-dihydro-1,1,3-trimethyl-1H-inden-4-yl)-1-methyl-1H-pyrazole-4-carboxamide, a compound represented by the following formula (1), a compound represented by the following formula (2), and a compound represented by the following formula (3),

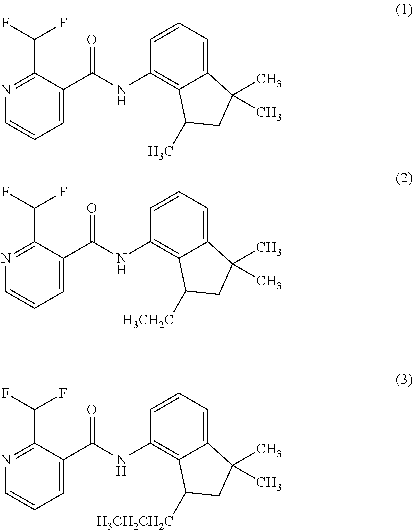

Sub-group (B-3) is a sterol biosynthesis inhibitor, which consists of prothioconazole and mefentrifluconazole,
Sub-group (B-4) is a group consisting of 3-(2-methylphenyl)butan-2-yl N-[(3-acetoxy-4-methoxypyridin-2-yl)carbonyl]alaninate, 3-(4-fluoro-2-methylphenyl)butan-2-yl N-[(3-acetoxy-4-methoxypyridin-2-yl)carbonyl]alaninate, 3-(4-methoxy-2-methylphenyl)butan-2-yl N-[(3-acetoxy-4-methoxypyridin-2-yl)carbonyl]alaninate, 3-(2,4-dimethylphenyl)butan-2-yl N-[(3-acetoxy-4-methoxypyridin-2-yl)carbonyl]alaninate, 3-(2-methylphenyl)butan-2-yl N-({3-[(2-methylpropanoyl)oxy]-4-methoxypyridin-2-yl}carbonyl)alaninate, 3-(4-fluoro-2-methylphenyl)butan-2-yl N-({3-[(2-methylpropanoyl)oxy]-4-methoxypyridin-2-yl}carbonyl)alaninate, 3-(4-methoxy-2-methylphenyl)butan-2-yl N-({3-[(2-methylpropanoyl)oxy]-4-methoxypyridin-2-yl}carbonyl)alaninate, 3-(2,4-dimethylphenyl)butan-2-yl N-({3-[(2-methylpropanoyl)oxy]-4-methoxypyridin-2-yl}carbonyl)alaninate, 3-(2-methylphenyl)butan-2-yl N-[(3-hydroxy-4-methoxypyridin-2-yl)carbonyl]alaninate, 3-(4-fluoro-2-methylphenyl)butan-2-yl N-[(3-hydroxy-4-methoxypyridin-2-yl)carbonyl]alaninate, 3-(4-methoxy-2-methylphenyl)butan-2-yl N-[(3-hydroxy-4-methoxypyridin-2-yl)carbonyl]alaninate, and 3-(2,4-dimethylphenyl)butan-2-yl N-[(3-hydroxy-4-methoxypyridin-2-yl)carbonyl]alaninate.

[16] The composition for controlling a plant disease according to [15], wherein the compound represented by formula (IV) is a compound wherein
E⁴ represents a phenyl group, a 2-methylphenyl group, a 3-methylphenyl group, a 4-methylphenyl group, a 3-fluorophenyl group, a 3-methoxyphenyl group, a 3-(trifluoromethoxy)phenyl group, or a 3-phenoxyphenyl group, and
Group (B) is a group consisting of the sub-group (B-1), the sub-group (B-2), and the sub-group (B-3).

[17] The composition for controlling a plant disease according to [16] wherein the compound represented by formula (IV) is

a compound wherein
A⁴ represents CH, L⁴ represents an oxygen atom, E⁴ represents a phenyl group, a 2-methylphenyl group, a 3-methylphenyl group, a 4-methylphenyl group, a 3-fluorophenyl group, a 3-methoxyphenyl group, a 3-(trifluoromethoxy)phenyl group, or a 3-phenoxyphenyl group, or
   a compound wherein
A⁴ represents a nitrogen atom, L⁴ represents NH, and E⁴ represents a 2-methylphenyl group, or a 3-fluorophenyl group.

[18] The composition for controlling a plant disease according to [16] or [17] wherein in the Group (B),
the sub-group (B-1) is a group consisting of pyraclostrobin, picoxystrobin, metyltetraprole, fenpicoxamid, and florylpicoxamid, and
the sub-group (B-2) is a group consisting of fluxapyroxad, benzovindiflupyr, fluindapyr, pydiflumetofen, 3-(difluoromethyl)-N-(2,3-dihydro-1,1,3-trimethyl-1H-inden-4-yl)-1-methyl-1H-pyrazole-4-carboxamide, the compound represented by formula (1), the compound represented by formula (2), and the compound represented by formula (3),

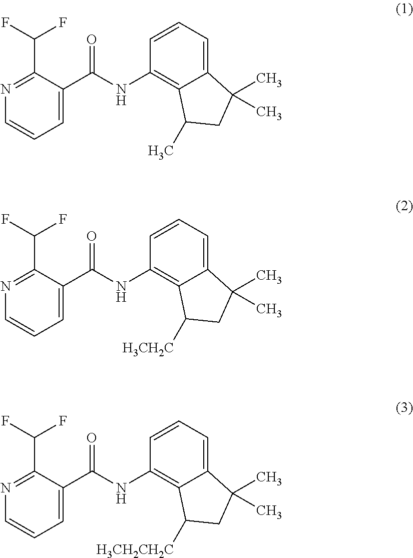

the sub-group (3) is a group consisting of prothioconazole and mefentrifluconazole.

[19] The composition for controlling a plant disease according to any one of [15] to [18] wherein the weight ratio of the compound represented by formula (IV) to one or more compounds selected from Group (B) is within a range of 1:0.01 to 1:100.

[20] The composition for controlling a plant disease according to any one of [15] to [18] wherein a weight ratio of the compound represented by formula (IV) to one of more compounds selected from Group (B) is within a range of 1:0.1 to 1:10.

[21] A method for controlling a plant disease which comprises applying an effective amount of the composition for controlling a plant disease according to any one of [15] to [20] to a plant or soil where a plant is cultivated.

[22] Use of any one of the composition for controlling a plant disease according to any one of [15] to [20].

### Effect of Invention

The present invention can control soybean rust fungus having an amino acid substitution of F129L on mitochondrial cytochrome b protein.

### MODE FOR CARRYING OUT THE INVENTION

The substituent(s) as described herein is/are explained.

The term “halogen atom” represents fluorine atom, chlorine atom, bromine atom, or iodine atom.

When the substituent has two or more halogen atoms, these halogen atoms may be identical to or different from each other.

The expression of “CX-CY” as used herein represents that the number of carbon atom is from X to Y. For example, the expression of “C1-C6” represents that the number of carbon atom is from 1 to 6.

The term “chain hydrocarbon group” represents an alkyl group, an alkenyl group, or an alkynyl group.

Examples of the “alkyl group” include methyl group, ethyl group, propyl group, isopropyl group, 1,1-dimethylpropyl group, 1,2-dimethylpropyl group, 1-ethylpropyl group, butyl group, sec-butyl group, tert-butyl group, pentyl group, and hexyl group.

Examples of the “alkenyl group” include vinyl group, 1-propenyl group, 2-propenyl group, 1-methyl-1-propenyl group, 1-methyl-2-propenyl group, 1,2-dimethyl-1-propenyl group, 1-ethyl-2-propenyl group, 3-butenyl group, 4-pentenyl group, and 5-hexenyl group.

Examples of the “alkynyl group” include ethynyl group, 1-propynyl group, 2-propynyl group, 1-methyl-2-propynyl group, 1,1-dimethyl-2-propynyl group, 1-ethyl-2-propynyl group, 2-butynyl group, 4-pentynyl group, and 5-hexynyl group.

Examples of the “alkoxy group” include methoxy group, ethoxy group, propoxy group, isopropoxy group, butoxy group, tert-butoxy group, pentyloxy group, and hexyloxy group.

Examples of the “alkylthio group” includes methylthio group, ethylthio group, propylthio group, isopropylthio group, butylthio group, pentylthio group, and hexylthio group.

Examples of the “alicyclic hydrocarbon group” include cycloalkyl group and cycloalkenyl group.

Examples of the “cycloalkyl group” include cyclopropyl group, cyclobutyl group, cyclopentyl group, cyclohexyl group, bicyclo[6.5.0]nonyl group, and bicyclo[6.6.0]decyl group.

Examples of the “cycloalkenyl group” include cyclopentenyl group, and cyclohexenyl group. Also the cycloalkenyl group may be fused by a benzene ring, and includes, for example, indanyl group and tetrahydronaphthyl group.

Examples of the “aryl group” include phenyl group, and naphthyl group.

Examples of the “aromatic heterocyclic group” include five-membered aromatic heterocyclic group such as pyrrolyl group, furanyl group, thienyl group, pyrazolyl group, imidazolyl group, triazolyl group, tetrazolyl group, oxazolyl group, isoxazolyl group, thiazolyl group, isothiazolyl group, oxadiazolyl group, and thiadiazolyl group and so on; six-membered aromatic heterocyclic group such as pyridyl group, pyridazinyl group, pyrimidinyl group, pyrazinyl group, triazinyl group, and tetrazinyl group and so on; nine-membered aromatic heterocyclic group such as indazolyl group, indolidinyl group, imidazopyridyl group and so on; and ten-membered aromatic heterocyclic group such as quinolyl group, isoquinolyl group, quinazolinyl group, naphthylidinyl group, and benzopyranyl group and so on.

Examples of the “non-aromatic heterocyclic group” include aziridinyl group, oxiranyl group, thiranyl group, azetidinyl group, oxetanyl group, thietanyl group, pyrrolidinyl group, tetrahydrofuranyl group, tetrahydrothienyl group, pyrazolynyl group, pyrazolidinyl group, imidazolinyl group, imidazolidinyl group, oxazolinyl group, thiazolinyl group, oxazolidinyl group, thiazolidinyl group, isoxazolinyl group, isoxazolidinyl group, isothiazolynyl group, isothiazolidinyl group, dioxolanyl group, dioxanyl group, piperidyl group, piperazinyl group, morpholinyl group, thiomorpholinyl group, pyranyl group, dihydropyranyl group, tetrahydropyranyl group, azepanyl group, oxepanyl group, thiepanyl group, dihydrobenzofuranyl group, 1,3-benzodioxolyl group, and a dihydrobenzopyranyl group.

The term(s) as described herein is/are explained.

The term of “soybean rust fungus having an amino acid substitution of F129L on mitochondrial cytochrome b protein” represents soybean rust fungus (scientific name: Phakopsora pachyrhizi) which shows a resistance against QoI fungicide by having a mutation in the mitochondrial cytochrome b gene encoding mitochondrial cytochrome protein and as a result of the mutation, causing amino acid substitution of F129L.

The present compound N, the present compound C, the present compound D, and the present compound A may be existed as one or more stereoisomers. Examples of the stereoisomer include enantiomer, diastereoisomer, atropisomer, and geometric isomer. Each stereoisomer, and stereoisomer mixture(s) in an arbitrary ratio of the present compound N, the present compound C, the present compound D, and the present compound A are included.

Examples of the geometric isomer include the following structures.

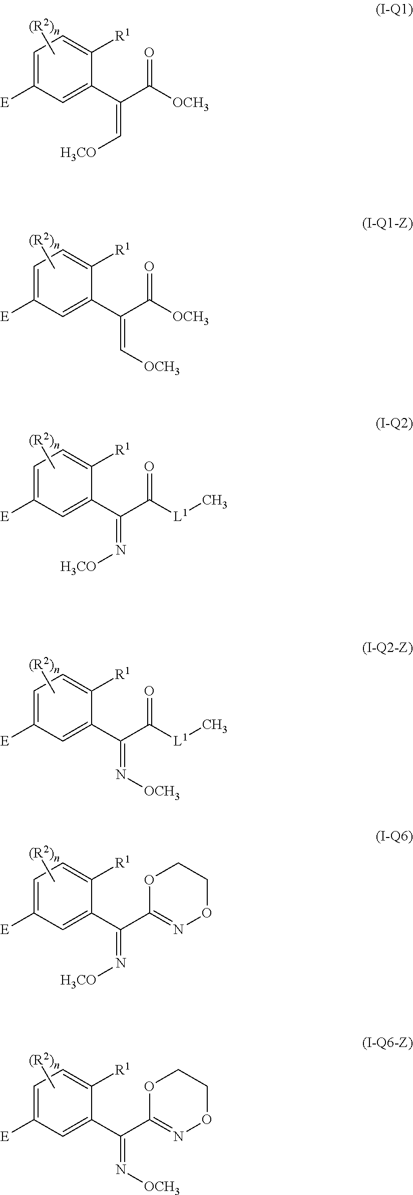

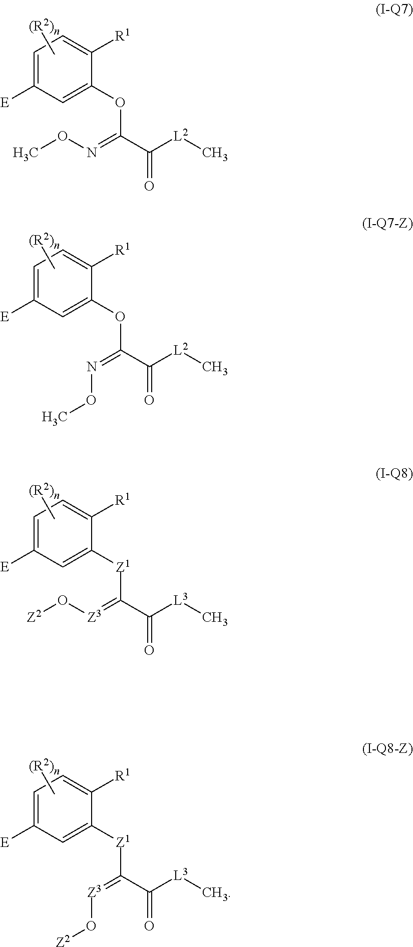

Examples of the agriculturally acceptable salt thereof include acid addition salts such as hydrochloride salts, sulfates, nitrates, phosphates, sulfonates, acetates, and benzoates.

Examples of the embodiment of the present compound N include the following compounds.

[Embodiment 1] The present compound N wherein E represents R²⁰—C≡C—, R²⁰ represents a C1-C6 chain hydrocarbon group which may be optionally substituted with one or more substituents selected from Group F, or a C3-C6 cycloalkyl group which may be optionally substituted with one or more halogen atoms.

[Embodiment 2] The present compound N wherein E represents R²⁰—C≡C—, R²⁰ represents a C1-C6 chain hydrocarbon group which may be optionally substituted with one or more halogen atoms, or a C3-C4 cycloalkyl group which may be optionally substituted with one or more halogen atoms.

[Embodiment 3] The present compound N wherein E represents R²⁰—C≡C—, R²⁰ represents a C1-C6 chain hydrocarbon group, or a C3-C4 cycloalkyl group.

[Embodiment 4] The present compound N wherein E represents R²⁰—C≡C—, R²⁰ represents a C1-C6 chain hydrocarbon group, or a cyclopropyl group.

[Embodiment 5] The present compound N wherein E represents a phenyl group which may be optionally substituted with one or more substituents selected from Group D.

[Embodiment 6] The present compound N wherein E represents a phenyl group which may be optionally substituted with one or more substituents selected from Group J1, and
Group J1 is a group consisting of a C1-C3 alkyl group, a C1-C3 alkoxy group {the C1-C3 alkyl group and the C1-C3 alkoxy group may be optionally substituted with one or more halogen atoms}, a halogen atom, a cyano group, and C(R⁷)═N—OR¹¹.

[Embodiment 7] The present compound N wherein E represents a group represented by formula (V)

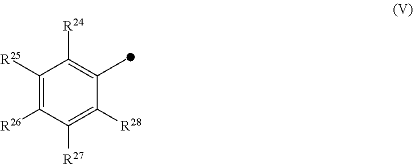

[wherein
R²⁴ and R²⁸ are identical to or different from each other and represent a halogen atom, or a hydrogen atom,
R²⁵ and R²⁷ are identical to or different from each other and represent a C1-C3 alkyl group which may be optionally substituted with one or more halogen atoms, a C1-C3 alkoxy group which may be optionally substituted with one or more halogen atoms, C(O) R^{11X}, C(R^{7X})═N—OR^{11X}, a halogen atom, a cyano group, or a hydrogen atom,
R^{7x} and R^{11x} are identical to or different from each other and represent a C1-C3 alkyl group, and
R²⁶ represents a C1-C3 alkyl group which may be optionally substituted with one or more halogen atoms, a C1-C3 alkoxy group which may be optionally substituted with one or more halogen atoms, a halogen atom, or a hydrogen atom.

[Embodiment 8] The present compound according to Embodiment 7 wherein R²⁴, R²⁶ and R²⁸ are identical to or different from each other and represent a fluorine atom or a hydrogen atom,
R²⁵ and R²⁷ are identical to or different from each other and represent C(R^{7X})═N—OR^{11X}, a halogen atom, a cyano group, or a hydrogen atom.

[Embodiment 9] The present compound N wherein E represents a five- to six-membered aromatic heterocyclic group which may be optionally substituted with one or more substituents selected from Group D.

[Embodiment 10] The present compound N wherein E represents a pyridyl group which may be optionally substituted with one or more substituents selected from Group J2, or a pyrimidinyl group which may be optionally substituted with one or more substituents selected from Group K,
Group J2 is a group consisting of a C1-C3 alkyl group, a C1-C3 alkoxy group {the C1-C3 alkyl group and the C1-C3 alkoxy group may be optionally substituted with one or more halogen atoms}, a halogen atom, and C(R^{7X})═N—OR^{11Y},
R^{11Y} represents a C1-C6 chain hydrocarbon group which may be optionally substituted with one or more substituents selected from Group F, and
Group K is a group consisting of a C1-C3 alkyl group which may be optionally substituted with one or more halogen atoms, a phenoxy group which may be optionally substituted with one or more substituents selected from Group G, and a halogen atom.

[Embodiment 11] The present compound N wherein E represents a pyridyl group which may be optionally substituted with one or more substituents selected from Group J3, or a pyrimidinyl group which may be optionally substituted with one or more substituents selected from Group K1,
Group J3 is a group consisting of a C1-C3 alkyl group, a C1-C3 alkoxy group {the C1-C3 alkyl group and the C1-C3 alkoxy group may be optionally substituted with one or more halogen atoms}, and a halogen atom,
Group K1 is a group consisting of a C1-C3 alkyl group which may be optionally substituted with one or more halogen atoms, a phenoxy group which may be optionally substituted with one or more substituents selected from Group G, and a halogen atom,
Group G1 is a group consisting of a cyano group and a halogen atom.

[Embodiment 12] The present compound N wherein E represents a thienyl group, a furanyl group, a thiadiazolyl group, or a pyrazolyl group {the thienyl group, the furanyl group, the thiadiazolyl group, and the pyrazolyl group may be optionally substituted with one or more substituents selected from Group J4},
Group J4 is a group consisting of a C1-C6 chain hydrocarbon group which may be optionally substituted with one or more substituents selected from Group F, and a C1-C6 alkoxy group which may be optionally substituted with one or more halogen atoms, and a halogen atom.

[Embodiment 13] The present compound N wherein E represents a thienyl group, a furanyl group, a thiadiazolyl group, or a pyrazolyl group {the thienyl group, the furanyl group, the thiadiazolyl group, and the pyrazolyl group may be optionally substituted with one or more substituents selected from Group J5}, and
Group J5 is a group consisting of a C1-C6 chain hydrocarbon group which may be optionally substituted with one or more substituents selected from Group F, and a halogen atom.

[Embodiment 14] The present compound N wherein E represents a group represented by formula (IV-1), a group represented by formula (IV-2), a group represented by formula (IV-3), a group represented by formula (IV-4), a group represented by formula (IV-5), a group represented by formula (IV-6), or a group represented by formula (IV-7),

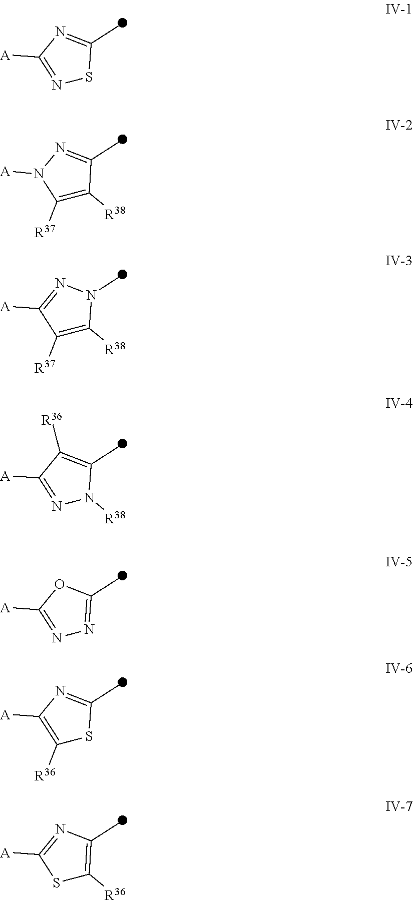

R³⁶ and R³⁷ are identical to or different from each other and represent a C1-C3 chain hydrocarbon group which may be optionally substituted with one or more halogen atoms, a halogen atom, or a hydrogen atom,
R³⁸ represents a C1-C3 chain hydrocarbon group which may be optionally substituted with one or more halogen atoms, or a hydrogen atom, and
A represents a phenyl group, or a pyridyl group {the phenyl group and the pyridyl group may be optionally substituted with one or more substituents selected from Group G}.

[Embodiment 15] The present compound N wherein E represents a group represented by formula (IV-1), a group represented by formula (IV-2), or a group represented by formula (IV-3), and A represents a phenyl group which may be optionally substituted with one or more halogen atoms.

[Embodiment 16] The present compound N wherein E represents a pyridyl group which may be optionally substituted with one or more substituents selected from Group J2, a pyrimidinyl group which may be optionally substituted with one or more substituents selected from Group K, a thienyl group, a furanyl group, a thiadiazolyl group, a pyrazolyl group {the thienyl group, the furanyl group, the thiadiazolyl group, and the pyrazolyl group may be optionally substituted with one or more substituents selected from Group J4}, a group represented by formula (IV-1), a group represented by formula (IV-2), a group represented by formula (IV-3), a group represented by formula (IV-4), a group represented by formula (IV-5), a group represented by formula (IV-6), or a group represented by formula (IV-7). [Embodiment 17] The present compound N wherein E represents a pyridyl group which may be optionally substituted with one or more substituents selected from Group J3, a pyrimidinyl group which may be optionally substituted with one or more substituents selected from Group K1, a thienyl group, a furanyl group, a thiadiazolyl group, a pyrazolyl group {the thienyl group, the furanyl group, the thiadiazolyl group, and the pyrazolyl group may be optionally substituted with one or more substituents selected from Group J5}, a group represented by formula (IV-1), a group represented by formula (IV-2), or a group represented by formula (IV-3), and A represents a phenyl group which may be optionally substituted with one or more halogen atoms.

[Embodiment 18] The present compound N wherein E represents R¹³O—N═C(R⁷)—, R⁷ represents a C1-C3 alkyl group, or a hydrogen atom, R¹³ represents a C1-C6 chain hydrocarbon group which may be optionally substituted with one or more substituents selected from Group F, a C3-C6 cycloalkyl group which may be optionally substituted with one or more halogen atoms, a phenyl group, a five- to six-membered aromatic heterocyclic group {the phenyl group and the five- to six-membered aromatic heterocyclic group may be optionally substituted with one or more substituents selected from Group G}, or R¹⁸—CH₂—, R¹⁸ represents a phenyl group, or a five- to six-membered aromatic heterocyclic group {the phenyl group and the five- to six-membered aromatic heterocyclic group may be optionally substituted with one or more substituents selected from Group G}.

[Embodiment 19] The present compound N wherein E represents R¹⁵—CH₂—O—N═C(R⁷)—, R⁷ represents a C1-C3 alkyl group, or a hydrogen atom, R¹⁵ represents a C1-C6 chain hydrocarbon group which may be optionally substituted with one or more substituents selected from Group F, a C3-C4 cycloalkyl group which may be optionally substituted with one or more halogen atoms, or R¹⁸—CH₂—, and R¹⁸ represents a phenyl group which may be optionally substituted with one or more substituents selected from Group G.

[Embodiment 20] The present compound N wherein E represents R¹⁵—CH₂—O—N═C(R⁷)—, R⁷ represents a methyl group, or a hydrogen atom, R¹⁵ represents a C1-C4 alkyl group which may be optionally substituted with one or more substituents selected from Group F, a C1-C4 alkyl group which may be optionally substituted with one or more substituents selected from Group F, a C3-C4 cycloalkyl group, or R¹⁸—CH₂—, and R¹⁸ represents a phenyl group {the phenyl group may be optionally substituted with one or more substituents selected from a C1-C3 alkyl group which may be optionally substituted with one or more halogen atoms and a halogen atom}.

[Embodiment 21] The present compound N wherein E represents R¹⁹O—, and R¹⁹ represents a C2-C6 chain hydrocarbon group which may be optionally substituted with one or more substituents selected from Group F, a C3-C4 cycloalkyl group which may be optionally substituted with one or more halogen atoms, or R¹⁷R⁷NC(O)—.

[Embodiment 22] The present compound N wherein E represents R¹⁹O—, and R¹⁹ represents a C2-C6 chain hydrocarbon group which may be optionally substituted with one or more halogen atoms.

[Embodiment 23] The present compound N wherein E represents R¹⁹O—, and R¹⁹ represents a C2-C6 chain hydrocarbon group.

[Embodiment 24] The present compound N wherein E represents, and R¹⁹ represents a C2-C6 alkyl group.

[Embodiment 25] The present compound N wherein E represents a C5-C6 cycloalkenyl group which may be optionally substituted with one or more substituents selected from Group B.

[Embodiment 26] The present compound N wherein E represents a C5-C6 cycloalkenyl group which may be optionally substituted with one or more halogen atoms.

[Embodiment 27] The present compound N wherein E represents a C5-C6 cycloalkenyl group.

[Embodiment 28] The present compound N wherein E represents a phenyl group which may be optionally substituted with one or more substituents selected from Group D, or a five- to six-membered aromatic heterocyclic group which may be optionally substituted with one or more substituents selected from Group D.

[Embodiment 29] The present compound N wherein E represents a phenyl group which may be optionally substituted with one or more substituents selected from Group J1, a pyridyl group which may be optionally substituted with one or more substituents selected from Group J2, a pyrimidinyl group which may be optionally substituted with one or more substituents selected from Group K, a thienyl group, a furanyl group, a thiadiazolyl group, a pyrazolyl group {the a thienyl group, the furanyl group, the thiadiazolyl group, and the pyrazolyl group may be optionally substituted with one or more substituents selected from Group J4}, a group represented by formula (IV-1), a group represented by formula (IV-2), a group represented by formula (IV-3), a group represented by formula (IV-4), a group represented by formula (IV-5), a group represented by formula (IV-6), or a group represented by formula (IV-7).

[Embodiment 30] The present compound N wherein E represents a phenyl group which may be optionally substituted with one or more substituents selected from Group J1, a pyridyl group which may be optionally substituted with one or more substituents selected from Group J3, a pyrimidinyl group which may be optionally substituted with one or more substituents selected from Group K1, a thienyl group, a furanyl group, a thiadiazolyl group, a pyrazolyl group {the a thienyl group, the furanyl group, the thiadiazolyl group, and the pyrazolyl group may be optionally substituted with one or more substituents selected from Group J5}, a group represented by formula (IV-1), a group represented by formula (IV-2), or a group represented by formula (IV-3), and A represents a phenyl group which may be optionally substituted with one or more halogen atoms.

[Embodiment 31] The present compound N wherein E represents a group represented by formula (V), a pyridyl group which may be optionally substituted with one or more substituents selected from Group J3, a pyrimidinyl group which may be optionally substituted with one or more substituents selected from Group K1, a thienyl group, a furanyl group, a thiadiazolyl group, a pyrazolyl group {the thienyl group, the furanyl group, the thiadiazolyl group, and the pyrazolyl group may be optionally substituted with one or more substituents selected from Group J5}, a group represented by formula (IV-1), a group represented by formula (IV-2), or a group represented by formula (IV-3), and A represents a phenyl group which may be optionally substituted with one or more halogen atoms.

[Embodiment 32] The present compound N wherein R²⁴, R²⁶ and R²⁸ are identical to or different from each other and represent a fluorine atom, or a hydrogen atom, and R²⁵ and R²⁷ are identical to or different from each other and represent C(R^{7X})═N—OR^{11X}, a halogen atom, a cyano group, or a hydrogen atom.

[Embodiment 33] The present compound N wherein E represents a phenyl group which may be optionally substituted with one or more substituents selected from Group D, a five- to six-membered aromatic heterocyclic group which may be optionally substituted with one or more substituents selected from Group D, or R²⁰—C≡C—, and R²⁰ represents a C1-C6 chain hydrocarbon group which may be optionally substituted with one or more substituents selected from Group, or a C3-C6 cycloalkyl group which may be optionally substituted with one or more halogen atoms.

[Embodiment 34] The present compound N wherein E represents a phenyl group which may be optionally substituted with one or more substituents selected from Group J1, a five- to six-membered aromatic heterocyclic group which may be optionally substituted with one or more substituents selected from Group I, or R²⁰—C≡C—, and R²⁰ represents a C1-C6 chain hydrocarbon group, or C3-C4 cycloalkyl group.

[Embodiment 35] The present compound N wherein E represents a phenyl group which may be optionally substituted with one or more substituents selected from Group J1, a pyridyl group which may be optionally substituted with one or more substituents selected from Group J2, a pyrimidinyl group which may be optionally substituted with one or more substituents selected from Group K, a thienyl group, a furanyl group, a thiadiazolyl group, a pyrazolyl group {the a thienyl group, the furanyl group, the thiadiazolyl group, and the pyrazolyl group may be optionally substituted with one or more substituents selected from Group J4}, a group represented by formula (IV-1), a group represented by formula (IV-2), a group represented by formula (IV-3), a group represented by formula (IV-4), a group represented by formula (IV-5), a group represented by formula (IV-6), a group represented by formula (IV-7), or R²⁰—C≡C—, and R²⁰ represents a C1-C6 chain hydrocarbon group, or a C3-C4 cycloalkyl group.

[Embodiment 36] The present compound N wherein E represents a phenyl group which may be optionally substituted with one or more substituents selected from Group J1, a pyridyl group which may be optionally substituted with one or more substituents selected from Group J3, a pyrimidinyl group which may be optionally substituted with one or more substituents selected from Group K1, a thienyl group, a furanyl group, a thiadiazolyl group, a pyrazolyl group {the a thienyl group, the furanyl group, the thiadiazolyl group, and the pyrazolyl group may be optionally substituted with one or more substituents selected from Group J5}, a group represented by formula (IV-1), a group represented by formula (IV-2), a group represented by formula (IV-3), or R²⁰—C≡C—, R²⁰ represents a C1-C6 alkyl group, or a cyclopropyl group, and A represents a phenyl group which may be optionally substituted with one or more halogen atoms.

[Embodiment 37] The present compound N wherein E represents a group represented by formula (V), a pyridyl group which may be optionally substituted with one or more substituents selected from Group J3, a pyrimidinyl group which may be optionally substituted with one or more substituents selected from Group K1, a thienyl group, a furanyl group, a thiadiazolyl group, a pyrazolyl group {the thienyl group, the furanyl group, the thiadiazolyl group, and the pyrazolyl group may be optionally substituted with one or more substituents selected from Group J5}, a group represented by formula (IV-1), a group represented by formula (IV-2), a group represented by formula (IV-3), or R²⁰—C≡C—, R²⁰ represents a C1-C6 alkyl group, or a cyclopropyl group, and A represents a phenyl group which may be optionally substituted with one or more halogen atoms.

[Embodiment 38] The compound according to [Embodiment 37] wherein R²⁴, R²⁶ and R²⁸ are identical to or different from each other and represent a fluorine atom or a hydrogen atom, and R²⁵ and R²⁷ are identical to or different from each other and represent C(R^{7X})═N—OR^{11X}, a halogen atom, a cyano group, or a hydrogen atom.

[Embodiment 39] The present compound N wherein E represents a phenyl group which may be optionally substituted with one or more substituents selected from Group D, a five- to six-membered aromatic heterocyclic group which may be optionally substituted with one or more substituents selected from Group D, a C5-C6 cycloalkenyl group which may be optionally substituted with one or more substituents selected from Group D, or R²⁰—C≡C—, and R²⁰ represents a C1-C6 chain hydrocarbon group which may be optionally substituted with one or more substituents selected from Group F, or a C3-C6 cycloalkyl group which may be optionally substituted with one or more halogen atoms.

[Embodiment 40] The present compound N wherein E represents a phenyl group which may be optionally substituted with one or more substituents selected from Group J1, a pyridyl group which may be optionally substituted with one or more substituents selected from Group J2, a pyrimidinyl group which may be optionally substituted with one or more substituents selected from Group K, a thienyl group, a furanyl group, a thiadiazolyl group, a pyrazolyl group {the thienyl group, the furanyl group, the thiadiazolyl group, and the pyrazolyl group may be optionally substituted with one or more substituents selected from Group J4}, a group represented by formula (IV-1), a group represented by formula (IV-2), a group represented by formula (IV-3), a group represented by formula (IV-4), a group represented by formula (IV-5), a group represented by formula (IV-6), a group represented by formula (IV-7), a C5-C6 cycloalkenyl group, or R²⁰—C≡C—, and R²⁰ represents a C1-C6 chain hydrocarbon group, or a C3-C4 cycloalkyl group.

[Embodiment 41] The present compound N wherein E represents a phenyl group which may be optionally substituted with one or more substituents selected from Group J1, a pyridyl group which may be optionally substituted with one or more substituents selected from Group J3, a pyrimidinyl group which may be optionally substituted with one or more substituents selected from Group K1, a thienyl group, a furanyl group, a thiadiazolyl group, a pyrazolyl group {the thienyl group, the furanyl group, the thiadiazolyl group, and the pyrazolyl group may be optionally substituted with one or more substituents selected from Group J5}, a group represented by formula (IV-1), a group represented by formula (IV-2), a group represented by formula (IV-3), a C5-C6 cycloalkenyl group, or R²⁰—C≡C—, R²⁰ represents a C1-C6 alkyl group, or a cyclopropyl group, and A represents a phenyl group which may be optionally substituted with one or more halogen atoms.

[Embodiment 42] The present compound N wherein E represents a group represented by formula (V), a pyridyl group which may be optionally substituted with one or more substituents selected from Group J3, a pyrimidinyl group which may be optionally substituted with one or more substituents selected from Group K1, a thienyl group, a furanyl group, a thiadiazolyl group, a pyrazolyl group {the thienyl group, the furanyl group, the thiadiazolyl group, and the pyrazolyl group may be optionally substituted with one or more substituents selected from Group J5}, a group represented by formula (IV-1), a group represented by formula (IV-2), a group represented by formula (IV-3), a C5-C6 cycloalkenyl group, or R²⁰—C≡C—, R²⁰ represents a C1-C6 alkyl group, or a cyclopropyl group, and A represents a phenyl group which may be optionally substituted with one or more halogen atoms.

[Embodiment 43] The compound according to [Embodiment 42] wherein R²⁴, R²⁵, and R²⁶ are identical to or different from each other and represent a fluorine atom or a hydrogen atom, R²⁵ and R²⁷ are identical to or different from each other and represent C(R^{7X})═N—OR^{11X}, a halogen atom, a cyano group, or a hydrogen atom.

[Embodiment 44] The present compound N wherein E represents a C1-C6 alkyl group which may be optionally substituted with one or more halogen atoms, a cyclopropyl group which may be optionally substituted with one or more substituents selected from Group B, or R⁴R⁵C═C(R⁶)—, R⁴ and R⁶ are identical to or different from each other and represent a C1-C3 alkyl group, a halogen atom, or a hydrogen atom, and R⁵ represents a C1-C6 chain hydrocarbon group which may be optionally substituted with one or more substituents selected from Group J, or a C3-C6 cycloalkyl group which may be optionally substituted with one or more halogen atoms.

[Embodiment 45] The present compound N wherein E represents R⁴R⁵C═C(R⁶)—, R⁴ and R⁶ represent a hydrogen atom, R⁵ represents a C1-C6 chain hydrocarbon group which may be optionally substituted with one or more substituents selected from Group J, or a C3-C6 cycloalkyl group which may be optionally substituted with one or more halogen atoms.

[Embodiment 46] The present compound N wherein E represents R⁴R⁵C═C(R⁶)—, R⁴ and R⁶ represent a hydrogen atom, R⁵ represents a C1-C6 chain hydrocarbon group, or a C3-C4 cycloalkyl group.

[Embodiment 47] The present compound N wherein E represents a R¹⁹O—, and R¹⁹ represents a phenyl group, or a five- to six-membered aromatic heterocyclic group {the phenyl group, and the five- to six-membered aromatic heterocyclic group may be optionally substituted with one or more substituents selected from Group G}.

[Embodiment 48] The present compound N wherein E represents R¹⁹O—, and R¹⁹ represents a phenyl group which may be optionally substituted with one or more substituents selected from Group G.

[Embodiment 49] The present compound N wherein E represents R¹⁹O—, and R¹⁹ represents a phenyl group which is substituted with one or more substituents selected from a halogen atom and a methyl group {with the proviso that a 2-methyl phenyl group, a 3-methylphenyl group, a 4-methylphenyl group, a 3,5-dimethylphenyl group, a 3-fluorophenyl group, a 3-bromophenyl group, and a 3-iodophenyl group are excluded}.

[Embodiment 50] The present compound N wherein E represents R¹⁹O—, and R¹⁹ represents a phenyl group which is substituted with one or more chlorine atoms.

[Embodiment 51] The present compound N wherein E represents R¹⁹O—, and R¹⁹ represents a phenyl group which is substituted with one or more fluorine atoms {with the proviso that a 3-fluorophenyl group is excluded}.

[Embodiment 52] The present compound N wherein E represents R¹⁹O—, and R¹⁹ represents a phenyl group which is substituted at its 2-position with a halogen atom, a phenyl group which is substituted at its 4-position with a halogen atom, a phenyl group which is substituted with two or more halogen atoms, or a 3-chlorophenyl group.

[Embodiment 53] The present compound N wherein E represents R¹⁹O—, and R¹⁹ represents a phenyl group which is substituted at its 2-position with a halogen atom.

[Embodiment 54] The present compound N wherein E represents R¹⁹O—, and R¹⁹ represents a phenyl group which is substituted at its 4-position with a halogen atom.

[Embodiment 55] The present compound N wherein E represents R¹⁹O—, and R¹⁹ represents a phenyl group which is substituted at its 2- or 4-position with a halogen atom.

[Embodiment 56] The present compound N wherein E represents R¹⁹O—, and R¹⁹ represents a phenyl group which is substituted at its 2-position with substituent(s) selected from Group L.

[Embodiment 57] The present compound N wherein E represents R¹⁹O—, and R¹⁹ represents a phenyl group which is substituted at its 3-position with substituent(s) selected from Group M.

[Embodiment 58] The present compound N wherein E represents R¹⁹O—, and R¹⁹ represents a phenyl group which is substituted at its 4-position with substituent(s) selected from Group N.

[Embodiment 59] The present compound N wherein E represents R¹⁹O—, and R¹⁹ represents a phenyl group which is substituted at its 2- or 4-position with substituent(s) selected from Group O,
Group O is a group consisting of a C2-C4 chain hydrocarbon group which may be optionally substituted with one or more halogen atoms, a methoxy group, a cyclopropyl group, a halogen atom, and a cyano group.

[Embodiment 60] The present compound N wherein E represents R¹⁹O—, and R¹⁹ represents a phenyl group which is substituted with two or more substituents selected from Group P.

[Embodiment 61] The present compound N wherein E represents R¹⁹O—, and R¹⁹ represents a phenyl group which is substituted at its 2-position with substituent(s) selected from Group L, a phenyl group which is substituted at its 3-position with substituent(s) selected from Group M, a phenyl group which is substituted at its 4-position with substituent(s) selected from Group N, or a phenyl group which is substituted with two or more substituents selected from Group P.

[Embodiment 62] The present compound N wherein E represents R¹⁹O—, and R¹⁹ represents a pyridyl group which may be optionally with substituent(s) selected from Group P, a pyrazolyl group which may be optionally substituted with substituent(s) selected from Group G, or a pyrimidinyl group which may be optionally substituted with substituent(s) selected from Group Q.

[Embodiment 63] The present compound N wherein E represents R¹⁹O—, and R¹⁹ represents a phenyl group which is substituted at its 2-position with substituent(s) selected from Group L a phenyl group which is substituted at its 3-position with substituent(s) selected from Group M, a phenyl group which is substituted at its 4-position with substituent(s) selected from Group N, a phenyl group which is substituted with two or more substituents selected from Group P, a pyridyl group which may be optionally with substituent(s) selected from Group P, a pyrazolyl group which may be optionally substituted with substituent(s) selected from Group G, or a pyrimidinyl group which may be optionally substituted with substituent(s) selected from Group Q.

[Embodiment 64] The present compound N wherein E represents R¹⁵—CH(R¹⁶)—O—N═C(R⁷)—, and R⁷ represents a C1-C3 alkyl group, R¹⁵ represents a phenyl group, or a five- to six-membered aromatic heterocyclic group {the phenyl group and the five- to six-membered aromatic heterocyclic group may be optionally substituted with one or more substituents selected from Group G}, and R¹⁶ represents a C1-C3 alkyl group.

[Embodiment 65] The present compound N wherein E represents R¹⁵—CH(R¹⁶)—O—N═C(R⁷)—, R⁷ represents a C1-C3 alkyl group, R¹⁵ represents a phenyl group which may be optionally substituted with one or more substituents selected from Group G, and R¹⁶ represents a C1-C3 alkyl group.

[Embodiment 66] The present compound N wherein E represents R¹⁵—CH(R¹⁶)—O—N═C(R⁷)—, R⁷ represents a methyl group, R⁵ represents a phenyl group {the phenyl group may be optionally substituted with one or more substituents selected from a C1-C3 alkyl group which may be optionally substituted with halogen atom and halogen atom}, and R¹⁶ represents a methyl group.

[Embodiment 67] The present compound N wherein E represents R¹⁵—CH(R¹⁶)—O—N═C(R⁷)—, R⁷ represents a hydrogen atom, R¹⁵ represents a phenyl group may be optionally substituted with one or more substituents selected from Group G6, and Group G6 is a group consisting of a C2-C3 chain hydrocarbon group which may be optionally substituted with one or more halogen atoms, a C1-C3 alkoxy group, a fluorine atom, or a cyano group, and R¹⁶ represents a C1-C3 alkyl group.

[Embodiment 68] The present compound N wherein E represents R¹⁵—CH(R¹⁶)—O—N═C(R⁷)—, and a combination of R⁷, R¹⁵ and R¹⁶ represents
a combination wherein R⁷ represents a C1-C3 alkyl group, or a cyclopropyl group, R¹⁵ represents a phenyl group which may be optionally substituted with one or more substituents selected from Group G, R¹⁶ represents a C1-C3 alkyl group, or
a combination wherein R⁷ represents a hydrogen atom, R¹⁵ represents a phenyl group may be optionally substituted with one or more substituents selected from Group G6, and R¹⁶ represents a C1-C3 alkyl group.

[Embodiment 69] The present compound N wherein E represents R³⁴R³⁵C═N—N═C(R¹⁰)—, R³⁴ represents a C1-C6 chain hydrocarbon group which may be optionally substituted with one or more substituents selected from Group J, a C3-C10 alicyclic hydrocarbon group which may be optionally substituted with one or more substituents selected from Group E, a phenyl group, or a five- to six-membered aromatic heterocyclic group {the phenyl group and the five- to six-membered aromatic heterocyclic group may be optionally substituted with one or more substituents selected from Group G}, R³⁵ represents a C1-C3 alkyl group, or a hydrogen atom,
R³⁴ and R³⁵ may combine together with a carbon atom to which they are attached to form a C3-C10 alicyclic hydrocarbon group, or a three- to ten-membered non-aromatic heterocyclic group {the C3-C10 alicyclic hydrocarbon group and the three- to ten-membered non-aromatic heterocyclic group may be optionally substituted with one or more substituents selected from Group E}.

[Embodiment 70] The present compound N wherein E represents R³⁴R³⁵C═N—N═C(R¹⁰)—. R³⁴ represents a phenyl group, or a five- to six-membered aromatic heterocyclic group {the phenyl group, and the five- to six-membered aromatic heterocyclic group may be optionally substituted with one or more substituents selected from Group G},
R³⁵ represents a C1-C3 alkyl group, or a hydrogen atom,
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, a 3,4-dihydro-2H-1-benzofuran-4-ylidene group, a 3,4-dihydro-2H-1-benzopyran-4-ylidene group, or a 3,4-dihydro-2H-1-benzopyran-4-ylidene group {the indan-1-ylidene group, the 1,2,3,4-tetrahydronaphthalen-1-yline group, the 2,3-dihydrobenzofuran-3-yline group, and the 3,4-dihydro-2H-1-benzofuran-4-ylidene group, the 3,4-dihydro-2H-1-benzopyran-4-ylidene group, and the 3,4-dihydro-2H-1-benzopyran-4-ylidene group may be optionally substituted with one or more substituents selected from Group G}.

[Embodiment 71] The present compound N wherein E represents R³⁴R³⁵C═N—N═C(R¹⁰)—, and R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, a 3,4-dihydro-2H-1-benzofuran-4-ylidene group, a 3,4-dihydro-2H-1-benzopyran-4-ylidene group, or a 3,4-dihydro-2H-1-benzopyran-4-ylidene group {the indan-1-ylidene group, the 1,2,3,4-tetrahydronaphthalen-1-yline group, the 2,3-dihydrobenzofuran-3-yline group, and the 3,4-dihydro-2H-1-benzofuran-4-ylidene group, the 3,4-dihydro-2H-1-benzopyran-4-ylidene group, and the 3,4-dihydro-2H-1-benzopyran-4-ylidene group may be optionally substituted with one or more substituents selected from Group G}.

[Embodiment 72] The present compound N wherein E represents R³⁴R³⁵C═N—N═C(R¹⁰)—, R³⁴ represents a C1-C6 chain hydrocarbon group which may be optionally substituted with one or more substituents selected from Group J, a C3-C6 cycloalkyl group which may be optionally substituted with one or more halogen atoms, a phenyl group, or a five- to six-membered aromatic heterocyclic group {the phenyl group and the five- to six-membered aromatic heterocyclic group may be optionally substituted with one or more substituents selected from Group G}, and R³⁵ represents a C1-C3 alkyl group, or a hydrogen atom.

[Embodiment 73] The present compound N wherein E represents R³⁴R³⁵C═N—N═C(R¹⁰)—, R³⁴ represents a phenyl group which may be optionally substituted with one or more substituents selected from Group G, and R³⁵ represents a methyl group.

[Embodiment 74] The present compound N wherein E represents R³⁴R³⁵C═N—N═C(R¹⁰)—, R³⁴ represents a phenyl group, a pyridyl group, a pyrazolyl group, or a thiazolyl group {the phenyl group, the pyridyl group, the pyrazolyl group, and the thiazolyl group may be optionally substituted with one or more substituents selected from Group G}, and R³⁵ represents a C1-C3 alkyl group, or
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, a 3,4-dihydro-2H-1-benzofuran-4-ylidene group, a 3,4-dihydro-2H-1-benzopyran-4-ylidene group, or a 3,4-dihydro-2H-1-benzopyran-4-ylidene group {the indan-1-ylidene group, the 1,2,3,4-tetrahydronaphthalen-1-yline group, the 2,3-dihydrobenzofuran-3-yline group, and the 3,4-dihydro-2H-1-benzofuran-4-ylidene group, the 3,4-dihydro-2H-1-benzopyran-4-ylidene group, and the 3,4-dihydro-2H-1-benzopyran-4-ylidene group may be optionally substituted with one or more substituents selected from Group G}.

[Embodiment 75] The present compound N wherein E represents R³⁴R³⁵C═N—N═C(R¹⁰)—, R³⁴ represents a phenyl group which may be optionally substituted with one or more substituents selected from Group G, and R³⁵ represents a C1-C3 alkyl group, or
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, or a 3,4-dihydro-2H-1-benzofuran-4-ylidene group {the indan-1-ylidene group, the 1,2,3,4-tetrahydronaphthalen-1-yline group, the 2,3-dihydrobenzofuran-3-yline group, and the 3,4-dihydro-2H-1-benzofuran-4-ylidene group may be optionally substituted with one or more substituents selected from Group G}.

[Embodiment 76] The present compound N wherein E represents R³⁴R³⁵C═N—N═C(R¹⁰)—, R³⁴ represents a phenyl group which may be optionally substituted with one or more substituents selected from Group G, and R³⁵ represents a C1-C3 alkyl group, or
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, or a 3,4-dihydro-2H-1-benzofuran-4-ylidene group {the indan-1-ylidene group, the 1,2,3,4-tetrahydronaphthalen-1-yline group, the 2,3-dihydrobenzofuran-3-yline group, and the 3,4-dihydro-2H-1-benzofuran-4-ylidene group may be optionally substituted with one or more substituents selected from Group J3}.

[Embodiment 77] The compound according to [Embodiment 69] wherein R¹⁰ represents a C1-C3 alkyl group, or a hydrogen atom.

[Embodiment 78] The compound according to [Embodiment 70] wherein R¹⁰ represents a C1-C3 alkyl group, or a hydrogen atom.

[Embodiment 79] The compound according to [Embodiment 71] wherein R¹⁰ represents a C1-C3 alkyl group, or a hydrogen atom.

[Embodiment 80] The compound according to [Embodiment 72] wherein R¹⁰ represents a C1-C3 alkyl group, or a hydrogen atom.

[Embodiment 81] The compound according to [Embodiment 73] wherein R¹⁰ represents a C1-C3 alkyl group, or a hydrogen atom.

[Embodiment 82] The compound according to [Embodiment 69] wherein R¹⁰ represents a C1-C3 alkyl group.

[Embodiment 83] The compound according to [Embodiment 70] wherein R¹⁰ represents a C1-C3 alkyl group.

[Embodiment 84] The compound according to [Embodiment 71] wherein R¹⁰ represents a C1-C3 alkyl group.

[Embodiment 85] The compound according to [Embodiment 72] wherein R¹⁰ represents a C1-C3 alkyl group.

[Embodiment 86] The compound according to [Embodiment 73] wherein R¹⁰ represents a C1-C3 alkyl group.

[Embodiment 87] The compound according to [Embodiment 74] wherein R¹⁰ represents a C1-C3 alkyl group.

[Embodiment 88] The compound according to [Embodiment 75] wherein R¹⁰ represents a C1-C3 alkyl group.

[Embodiment 89] The compound according to [Embodiment 76] wherein R¹⁰ represents a C1-C3 alkyl group.

[Embodiment 90] The compound according to [Embodiment 69] wherein R¹⁰ represents a methyl group.

[Embodiment 91] The compound according to [Embodiment 70] wherein R¹⁰ represents a methyl group.

[Embodiment 92] The compound according to [Embodiment 71] wherein R¹⁰ represents a methyl group.

[Embodiment 93] The compound according to [Embodiment 72] wherein R¹⁰ represents a methyl group.

[Embodiment 94] The compound according to [Embodiment 73] wherein R¹⁰ represents a methyl group.

[Embodiment 95] The compound according to [Embodiment 74] wherein R¹⁰ represents a methyl group.

[Embodiment 96] The compound according to [Embodiment 75] wherein R¹⁰ represents a methyl group.

[Embodiment 97] The compound according to [Embodiment 76] wherein R¹⁰ represents a methyl group.

[Embodiment 98] The compound according to [Embodiment 70] wherein R¹⁰ represents a hydrogen atom.

[Embodiment 99] The compound according to [Embodiment 71] wherein R¹⁰ represents a hydrogen atom.

[Embodiment 100] The compound according to [Embodiment 72] wherein R¹⁰ represents a hydrogen atom.

[Embodiment 101] The compound according to [Embodiment 73] wherein R¹⁰ represents a hydrogen atom.

[Embodiment 102] The present compound N wherein E represents R⁸R⁹C═N—O—CH₂—, R⁸ and R⁹ may combine together with a carbon atom to which they are attached to form a C3-C10 alicyclic hydrocarbon group, or a three- to ten-membered non-aromatic heterocyclic group {the C3-C10 alicyclic hydrocarbon group and the three- to ten-membered non-aromatic heterocyclic group may be optionally substituted with one or more substituents selected from Group E}.

[Embodiment 103] The present compound N wherein E represents R⁸R⁹C═N—O—CH₂—,
R⁸ represents a C1-C6 chain hydrocarbon group which may be optionally substituted with one or more substituents selected from Group J, a C3-C10 alicyclic hydrocarbon group which may be optionally substituted with one or more substituents selected from Group E, a phenyl group, or a five- to six-membered aromatic heterocyclic group {the phenyl group and the five- to six-membered aromatic heterocyclic group may be optionally substituted with one or more substituents selected from Group G}, and
R⁹ represents a C1-C3 alkyl group, or a hydrogen atom.

[Embodiment 104] The present compound N wherein E represents R⁸R⁹C═N—O—CH₂—,
R⁸ represents a phenyl group which may be optionally substituted with one or more substituents selected from Group G, and
R⁹ represents a methyl group.

[Embodiment 105] The present compound N wherein E represents R⁸R⁹C═N—O—CH₂—,
R⁸ represents an indanyl group, a tetrahydronaphthyl group, a dihydrobenzofuranyl group, or a dihydrobenzopyranyl group {the indanyl group, the tetrahydronaphthyl group, the dihydrobenzofuranyl group, and the dihydrobenzopyranyl group may be optionally substituted with one or more substituents selected from Group G}, and
R⁹ represents a C1-C3 alkyl group, or a hydrogen atom,
R⁸ and R⁹ may combine together with a carbon atom to which they are attached to form an indan-1-ylidene group, a 1,2,3,4-tetrahydronaphthalen-1-yline group, a 2,3-dihydrobenzofuran-3-yline group, a 3,4-dihydro-2H-1-benzofuran-4-ylidene group, a 3,4-dihydro-2H-1-benzopyran-4-ylidene group, or a 3,4-dihydro-2H-1-benzopyran-4-ylidene group {the indan-1-ylidene group, the 1,2,3,4-tetrahydronaphthalen-1-yline group, the 2,3-dihydrobenzofuran-3-yline group, the 3,4-dihydro-2H-1-benzofuran-4-ylidene group, the 3,4-dihydro-2H-1-benzopyran-4-ylidene group, and the 3,4-dihydro-2H-1-benzopyran-4-ylidene group may be optionally substituted with one or more substituents selected from Group G}.

[Embodiment 106] The present compound N wherein E represents R⁸R⁹C═N—O—CH₂—,
R⁸ represents an indanyl group, a tetrahydronaphthyl group, a dihydrobenzofuranyl group, or a dihydrobenzopyranyl group {the indanyl group, the tetrahydronaphthyl group, the dihydrobenzofuranyl group, and the dihydrobenzopyranyl group may be optionally substituted with one or more substituents selected from Group J3}, and
R⁹ represents a C1-C3 alkyl group.

[Embodiment 107] The present compound N wherein E represents R⁸R⁹C═N—O—CH₂—,
R⁸ represents an indanyl group, or a tetrahydronaphthyl group {the indanyl group, and the tetrahydronaphthyl group may be optionally substituted with one or more substituents selected from Group J3}, and
R⁹ represents a C1-C3 alkyl group.

[Embodiment 108] The present compound N wherein E represents R⁸R⁹C═N—O—CH₂—,
R⁸ represents an indanyl group, a tetrahydronaphthyl group, a dihydrobenzofuranyl group, or a dihydrobenzopyranyl group {the indanyl group, the tetrahydronaphthyl group, the dihydrobenzofuranyl group, and the dihydrobenzopyranyl group may be optionally substituted with one or more substituents selected from Group G}, and
R⁹ represents a C1-C3 alkyl group,
R⁸ and R⁹ may combine together with a carbon atom to which they are attached to form a C3-C10 alicyclic hydrocarbon group, or a three- to ten-membered non-aromatic heterocyclic group {the C3-C10 alicyclic hydrocarbon group and the three- to ten-membered non-aromatic heterocyclic group may be optionally substituted with one or more substituents selected from Group E}.

[Embodiment 109] The present compound N wherein E represents R⁸R⁹C═N—O—CH₂—, and
R⁸ and R⁹ may combine together with a carbon atom to which they are attached to form an indan-1-ylidene group, a 1,2,3,4-tetrahydronaphthalen-1-yline group, a 2,3-dihydrobenzofuran-3-yline group, a 3,4-dihydro-2H-1-benzofuran-4-ylidene group, a 3,4-dihydro-2H-1-benzopyran-4-ylidene group, or a 3,4-dihydro-2H-1-benzopyran-4-ylidene group {the indan-1-ylidene group, the 1,2,3,4-tetrahydronaphthalen-1-yline group, the 2,3-dihydrobenzofuran-3-yline group, the 3,4-dihydro-2H-1-benzofuran-4-ylidene group, the 3,4-dihydro-2H-1-benzopyran-4-ylidene group, and the 3,4-dihydro-2H-1-benzopyran-4-ylidene group may be optionally substituted with one or more substituents selected from Group J3}.

[Embodiment 110] The present compound N wherein E represents T-O—CH₂—, and
T represents a phenyl group, or a five- to six-membered aromatic heterocyclic group {the phenyl group and the five- to six-membered aromatic heterocyclic group which may be optionally substituted with one or more substituents selected from Group D1},
Group D1 is a group consisting of a C1-C6 chain hydrocarbon group which may be optionally substituted with one or more substituents selected from Group F, a C3-C6 cycloalkyl group which may be optionally substituted with one or more substituents selected from Group B, a C1-C3 alkoxy group which may be optionally substituted with one or more halogen atoms, a phenyl group, a five- to six-membered aromatic heterocyclic group {the phenyl group and the five- to six-membered aromatic heterocyclic group may be optionally substituted with one or more substituents selected from Group G}, a halogen atom, and a cyano group.

[Embodiment 111] The present compound N wherein E represents T-O—CH₂—, and
T represents a phenyl group, a pyridin-2-yl group {the phenyl group and the pyridin-2-yl group may be optionally substituted with one or more substituents selected from Group D2}, a 1-phenyl-1H-pyrazol-3-yl group, or a 1-methyl-3-phenyl-1H-pyrazol-5-yl group {the phenyl group in the 1-phenyl-1H-pyrazol-3-yl group and the 1-methyl-3-phenyl-1H-pyrazol-5-yl group are identical to or different from each other and may be optionally substituted with one or more substituents selected from Group D3, and the pyrazolyl group in the 1-phenyl-1H-pyrazol-3-yl group and the 1-methyl-3-phenyl-1H-pyrazol-5-yl group are identical to or different from each other and may be optionally substituted with one or more substituents selected from Group D2},
Group D2 is a group consisting of a C1-C3 alkyl group which may be optionally substituted with one or more halogen atoms and a halogen atom,
Group D3 is a group consisting of a C1-C3 alkyl group which may be optionally substituted with one or more halogen atoms, a C1-C3 alkoxy group which may be optionally substituted with one or more halogen atoms, a cyano group and a halogen atom.

[Embodiment 112] The present compound N wherein E represents R⁴R⁵C═C(R⁶)—, R¹⁵—CH(R¹⁶)—O—N═C(R⁷)—, or R¹⁹O—,
R⁴ and R⁶ represent a hydrogen atom, and R⁵ represents a C1-C6 chain hydrocarbon group, or a C3-C4 cycloalkyl group,
R⁷ represents a methyl group, or a hydrogen atom, R¹⁵ represents a phenyl group which may be optionally substituted with one or more substituents selected from Group G, and R¹⁶ represents a methyl group, and
R¹⁹ represents a phenyl group which is substituted with one or more fluorine atoms [with the proviso that a 3-fluorophenyl group is excluded}.

[Embodiment 113] The present compound N wherein E represents R⁴R⁵C═C(R⁶)—, R¹⁵—CH(R¹⁶)—O—N═C(R⁷)—, R⁸R⁹C═N—O—CH₂—, or R¹⁹O—,
R⁴ and R⁶ represent a hydrogen atom, and R⁵ represents a C1-C6 chain hydrocarbon group, or C3-C4 cycloalkyl group,
R⁷ represents a methyl group, or a hydrogen atom, R¹⁵ represents a phenyl group which may be optionally substituted with one or more substituents selected from Group G, and R¹⁶ represents a methyl group,
R⁸ represents a phenyl group which may be optionally substituted with one or more substituents selected from Group G, and R⁹ represents a methyl group,
R¹⁹ represents a phenyl group which is substituted with one or more fluorine atoms {with the proviso that a 3-fluorophenyl group is excluded}.

[Embodiment 114] The present compound N wherein E represents R⁴R⁵C═C(R⁶)—, R¹⁵—CH(R¹⁶)—O—N═C(R⁷)—, R⁸R⁹C═N—O—CH₂—, T-O—CH₂— or R¹⁹O—,
R⁴ and R⁶ represent a hydrogen atom, and R⁵ represents a C1-C6 chain hydrocarbon group, or a C3-C4 cycloalkyl group,
R⁷ represents a methyl group, or a hydrogen atom, R¹⁵ represents a phenyl group which may be optionally substituted with one or more substituents selected from Group G, and R¹⁶ represents a methyl group,
R⁸ represents a phenyl group which may be optionally substituted with one or more substituents selected from Group G, and R⁹ represents a methyl group,
R¹⁹ represents a phenyl group which is substituted with one or more fluorine atoms [with the proviso that a 3-fluorophenyl group is excluded},
T represents a phenyl group, a pyridin-2-yl group {the phenyl group and the pyridin-2-yl group may be optionally substituted with one or more substituents selected from Group D2}, a 1-phenyl-1H-pyrazol-3-yl group, or a 1-methyl-3-phenyl-1H-pyrazol-5-yl group {the phenyl group in the 1-phenyl-1H-pyrazol-3-yl group and the 1-methyl-3-phenyl-1H-pyrazol-5-yl group are identical to or different from each other and may be optionally substituted with one or more substituents selected from Group D3, and the pyrazolyl group in the 1-phenyl-1H-pyrazol-3-yl group and the 1-methyl-3-phenyl-1H-pyrazol-5-yl group are identical to or different from each other and may be optionally substituted with one or more substituents selected from Group D2}.

[Embodiment 115] The present compound N wherein E represents R⁴R⁵C═C(R⁶)—, R¹⁵—CH(R¹⁶)—O—N═C(R⁷)—, R⁸R⁹C═N—O—CH₂—, T-O—CH₂—, R³⁴R³⁵C═N—N═C(R¹⁰)—, or R¹⁹O—,
R⁴ and R⁶ represent a hydrogen atom, R⁵ represents a C1-C6 chain hydrocarbon group, or a C3-C4 cycloalkyl group,
R⁷ represents a methyl group, or a hydrogen atom, R¹⁵ represents a phenyl group which may be optionally substituted with one or more substituents selected from Group G, and R¹⁶ represents a methyl group,
R⁸ represents a phenyl group which may be optionally substituted with one or more substituents selected from Group G, R⁹ represents a methyl group,
R¹⁹ represents a phenyl group which is substituted with one or more fluorine atoms {with the proviso that a 3-fluorophenyl group is excluded},
T represents a phenyl group a pyridin-2-yl group {the phenyl group, and the pyridin-2-yl group may be optionally substituted with one or more substituents selected from Group D2}, a 1-phenyl-1H-pyrazol-3-yl group, or a 1-methyl-3-phenyl-1H-pyrazol-5-yl group {the phenyl group in the 1-phenyl-1H-pyrazol-3-yl group and the 1-methyl-3-phenyl-1H-pyrazol-5-yl group are identical to or different from each other and may be optionally substituted with one or more substituents selected from Group D3, and the pyrazolyl group in the 1-phenyl-1H-pyrazol-3-yl group and the 1-methyl-3-phenyl-1H-pyrazol-5-yl group are identical to or different from each other and may be optionally substituted with one or more substituents selected from Group D2},
R³⁴ represents a phenyl group which may be optionally substituted with one or more substituents selected from Group G,
R³⁵ represents a C1-C3 alkyl group, or a hydrogen atom,
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, a 3,4-dihydro-2H-1-benzofuran-4-ylidene group, a 3,4-dihydro-2H-1-benzopyran-4-ylidene group, a 1-oxo-3,4-dihydro-2H-1-benzopyran-4-ylidene group, a 3,4-dihydro-2H-1-benzopyran-4-ylidene group, or a 1,1-dioxo-3,4-dihydro-2H-1-benzopyran-4-ylidene group {the indan-1-ylidene group, the 1,2,3,4-tetrahydronaphthalen-1-yline group, the 2,3-dihydrobenzofuran-3-yline group, and the 3,4-dihydro-2H-1-benzofuran-4-ylidene group, the 3,4-dihydro-2H-1-benzopyran-4-ylidene group, the 1-oxo-3,4-dihydro-2H-1-benzopyran-4-ylidene group, the 3,4-dihydro-2H-1-benzopyran-4-ylidene group, and the 1,1-dioxo-3,4-dihydro-2H-1-benzopyran-4-ylidene group may be optionally substituted with one or more substituents selected from Group G},
R¹⁰ represents a C1-C3 alkyl group, or a hydrogen atom.

[Embodiment 116] The present compound N wherein E represents R³⁴R³⁵C═N—N═C(R¹⁰)—, R⁸R⁹C═N—O—CH₂—, R¹⁵—CH(R¹⁶)—O—N═C(R⁷)—, or R¹⁹O—,
R³⁴ represents a C1-C6 chain hydrocarbon group which may be optionally substituted with one or more substituents selected from Group J, a C3-C10 alicyclic hydrocarbon group which may be optionally substituted with one or more substituents selected from Group E, a phenyl group, or a five- to six-membered aromatic heterocyclic group {the phenyl group and the five- to six-membered aromatic heterocyclic group may be optionally substituted with one or more substituents selected from Group G},
R⁹ and R³⁵ are identical to or different from each other and represent a C1-C3 alkyl group, or a hydrogen atom,
R⁷, R¹⁰ and R¹⁶ are identical to or different from each other and represent a C1-C3 alkyl group,
R⁸ represents an indanyl group, a tetrahydronaphthyl group, a dihydrobenzofuranyl group, or a dihydrobenzopyranyl group {the indanyl group, the tetrahydronaphthyl group, the dihydrobenzofuranyl group, and the dihydrobenzopyranyl group may be optionally substituted with one or more substituents selected from Group G},
R⁸ and R⁹ may combine together with a nitrogen atom to which they are attached to form a C3-C10 alicyclic hydrocarbon group, or a three- to ten-membered non-aromatic heterocyclic group {the C3-C10 alicyclic hydrocarbon group and the three- to ten-membered non-aromatic heterocyclic group may be optionally substituted with one or more substituents selected from Group E},
R³⁴ and R³⁵ may combine together with a carbon atom to which they are attached to form a C3-C10 alicyclic hydrocarbon group, or a three- to ten-membered non-aromatic heterocyclic group {the C3-C10 alicyclic hydrocarbon group, and the three- to ten-membered non-aromatic heterocyclic group may be optionally substituted with one or more substituents selected from Group E},
R¹⁵ represents a phenyl group, or a five- to six-membered aromatic heterocyclic group {the phenyl group and the five- to six-membered aromatic heterocyclic group may be optionally substituted with one or more substituents selected from Group G},
R¹⁹ represents a phenyl group which is substituted at its 2-position with substituent(s) selected from Group L a phenyl group which is substituted at its 3-position with substituent(s) selected from Group M, a phenyl group which is substituted at its 4-position with substituent(s) selected from Group N, a phenyl group which is substituted with two or more substituents selected from Group P (the two or more substituents selected from Group P may be identical to or different from each other), a pyridyl group which may be optionally with substituent(s) selected from Group P, A pyrazolyl group which may be optionally substituted with substituent(s) selected from Group G, or a pyrimidinyl group which may be optionally substituted with substituent(s) selected from Group Q.

[Embodiment 117] The present compound N wherein E represents R³⁴R³⁵C═N—N═C(R¹⁰)—, R⁸R⁹C═N—O—CH₂—, or R¹⁹O—,
R³⁴ represents a C1-C6 chain hydrocarbon group which may be optionally substituted with one or more substituents selected from Group J, a C3-C10 alicyclic hydrocarbon group which may be optionally substituted with one or more substituents selected from Group E, a phenyl group, or a five- to six-membered aromatic heterocyclic group {the phenyl group and the five- to six-membered aromatic heterocyclic group may be optionally substituted with one or more substituents selected from Group G},
R⁹ and R³⁵ are identical to or different from each other and represent a C1-C3 alkyl group, or a hydrogen atom,
R¹⁰ represents a C1-C3 alkyl group,
R⁸ represents an indanyl group, a tetrahydronaphthyl group, a dihydrobenzofuranyl group, or a dihydrobenzopyranyl group {the indanyl group, the tetrahydronaphthyl group, the dihydrobenzofuranyl group, and the dihydrobenzopyranyl group may be optionally substituted with one or more substituents selected from Group G},
R⁸ and R⁹ may combine together with a nitrogen atom to which they are attached to form a C3-C10 alicyclic hydrocarbon group, or a three- to ten-membered non-aromatic heterocyclic group{the C3-C10 alicyclic hydrocarbon group and the three- to ten-membered non-aromatic heterocyclic group may be optionally substituted with one or more substituents selected from Group E},
R³⁴ and R³⁵ may combine together with a carbon atom to which they are attached to form a C3-C10 alicyclic hydrocarbon group, or a three- to ten-membered non-aromatic heterocyclic group {the C3-C10 alicyclic hydrocarbon group and the three- to ten-membered non-aromatic heterocyclic group may be optionally substituted with one or more substituents selected from Group E},
R¹⁹ represents a phenyl group which is substituted at its 2-position with substituent(s) selected from Group L, a phenyl group which is substituted at its 3-position with substituent(s) selected from Group M, a phenyl group which is substituted at its 4-position with substituent(s) selected from Group N, a phenyl group which is substituted with two or more substituents selected from Group P (the two or more substituents selected from Group P may be identical to or different from each other), a pyridyl group which may be optionally with substituent(s) selected from Group P, a pyrazolyl group which may be optionally substituted with substituent(s) selected from Group G, or a pyrimidinyl group which may be optionally substituted with substituent(s) selected from Group Q.

[Embodiment 118] The present compound N wherein E represents R³⁴R³⁵C═N—N═C(R¹⁰)—, or R¹⁹O—,
R³⁴ represents a C1-C6 chain hydrocarbon group which may be optionally substituted with one or more substituents selected from Group J, a C3-C10 alicyclic hydrocarbon group which may be optionally substituted with one or more substituents selected from Group E, a phenyl group, or a five- to six-membered aromatic heterocyclic group {the phenyl group and the five- to six-membered aromatic heterocyclic group may be optionally substituted with one or more substituents selected from Group G},
R³⁵ represents a C1-C3 alkyl group, or a hydrogen atom,
R³⁴ and R³⁵ may combine together with a carbon atom to which they are attached to form, C3-C10 alicyclic hydrocarbon group, or a three- to ten-membered non-aromatic heterocyclic group{the C3-C10 alicyclic hydrocarbon group and the three- to ten-membered non-aromatic heterocyclic group may be optionally substituted with one or more substituents selected from Group E},
R¹⁰ represents a C1-C3 alkyl group,
R¹⁹ represents a phenyl group which is substituted at its 2-position with substituent(s) selected from Group L, a phenyl group which is substituted at its 3-position with substituent(s) selected from Group M, a phenyl group which is substituted at its 4-position with substituent(s) selected from Group N, a phenyl group which is substituted with two or more substituents selected from Group P (the two or more substituents selected from Group P may be identical to or different from each other), a pyridyl group which may be optionally with substituent(s) selected from Group P, a pyrazolyl group which may be optionally substituted with substituent(s) selected from Group G, or a pyrimidinyl group which may be optionally substituted with substituent(s) selected from Group Q.

[Embodiment 119] The present compound N wherein E represents R⁸R⁹C═N—O—CH₂—, or R¹⁹O—,
R⁸ represents an indanyl group, a tetrahydronaphthyl group, a dihydrobenzofuranyl group, or a dihydrobenzopyranyl group {the indanyl group, the tetrahydronaphthyl group, the dihydrobenzofuranyl group, and the dihydrobenzopyranyl group may be optionally substituted with one or more substituents selected from Group G}, and R⁹ represents a C1-C3 alkyl group, or
R⁸ and R⁹ may combine together with a nitrogen atom to which they are attached to form an indan-1-ylidene group, a 1,2,3,4-tetrahydronaphthalen-1-yline group, a 2,3-dihydrobenzofuran-3-yline group, a 3,4-dihydro-2H-1-benzofuran-4-ylidene group, a 3,4-dihydro-2H-1-benzopyran-4-ylidene group, or a 3,4-dihydro-2H-1-benzopyran-4-ylidene group {the indan-1-ylidene group, the 1,2,3,4-tetrahydronaphthalen-1-yline group, the 2,3-dihydrobenzofuran-3-yline group, the 3,4-dihydro-2H-1-benzofuran-4-ylidene group, the 3,4-dihydro-2H-1-benzopyran-4-ylidene group, and the 3,4-dihydro-2H-1-benzopyran-4-ylidene group may be optionally substituted with one or more substituents selected from Group G},
R¹⁹ represents a phenyl group which is substituted at its 2-position with substituent(s) selected from Group L a phenyl group which is substituted at its 3-position with substituent(s) selected from Group M, a phenyl group which is substituted at its 4-position with substituent(s) selected from Group N, a phenyl group which is substituted with two or more substituents selected from Group P (the two or more substituents selected from Group P may be identical to or different from each other), a pyridyl group which may be optionally with substituent(s) selected from Group P, a pyrazolyl group which may be optionally substituted with substituent (s) selected from Group G, or a pyrimidinyl group which may be optionally substituted with substituent(s) selected from Group Q.

[Embodiment 120] The present compound N wherein E represents R³⁴R³⁵C═N—N═C(R¹⁰)—, or R⁸R⁹C═N—O—CH₂—,
R³⁴ represents a C1-C6 chain hydrocarbon group which may be optionally substituted with one or more substituents selected from Group J, a C3-C6 cycloalkyl group which may be optionally substituted with one or more halogen atoms, a phenyl group, or a five- to six-membered aromatic heterocyclic group {the phenyl group and the five- to six-membered aromatic heterocyclic group may be optionally substituted with one or more substituents selected from Group G},
R³⁵ represents a C1-C3 alkyl group, or a hydrogen atom,
R³⁴ and R³⁵ may combine together with a carbon atom to which they are attached to form a C3-C10 alicyclic hydrocarbon group, or a three- to ten-membered non-aromatic heterocyclic group {the C3-C10 alicyclic hydrocarbon group and the three- to ten-membered non-aromatic heterocyclic group may be optionally substituted with one or more substituents selected from Group E},
R¹⁰ represents a C1-C3 alkyl group,
R⁸ represents an indanyl group, a tetrahydronaphthyl group, a dihydrobenzofuranyl group, or a dihydrobenzopyranyl group {the indanyl group, the tetrahydronaphthyl group, the dihydrobenzofuranyl group, and the dihydrobenzopyranyl group may be optionally substituted with one or more substituents selected from Group G}, and R⁹ represents a C1-C3 alkyl group, or
R⁸ and R⁹ may combine together with a nitrogen atom to which they are attached to form an indan-1-ylidene group, a 1,2,3,4-tetrahydronaphthalen-1-yline group, a 2,3-dihydrobenzofuran-3-yline group, a 3,4-dihydro-2H-1-benzofuran-4-ylidene group, a 3,4-dihydro-2H-1-benzopyran-4-ylidene group, or a 3,4-dihydro-2H-1-benzopyran-4-ylidene group {the indan-1-ylidene group, the 1,2,3,4-tetrahydronaphthalen-1-yline group, the 2,3-dihydrobenzofuran-3-yline group, the 3,4-dihydro-2H-1-benzofuran-4-ylidene group, the 3,4-dihydro-2H-1-benzopyran-4-ylidene group, and the 3,4-dihydro-2H-1-benzopyran-4-ylidene group may be optionally substituted with one or more substituents selected from Group G}.

[Embodiment 121] The present compound N wherein E represents R³⁴R³⁵C═N—N═C(R¹⁰)—, R¹⁵—CH(R¹⁶)—O—N═C(R⁷)—, or R¹⁹O—,
R³⁴ represents a C1-C6 chain hydrocarbon group which may be optionally substituted with one or more substituents selected from Group J, a C3-C6 cycloalkyl group which may be optionally substituted with one or more halogen atoms, a phenyl group, or a five- to six-membered aromatic heterocyclic group {the phenyl group and the five- to six-membered aromatic heterocyclic group may be optionally substituted with one or more substituents selected from Group G},
R³⁵ represents a C1-C3 alkyl group, or a hydrogen atom,
R³⁴ and R³⁵ may combine together with a carbon atom to which they are attached to form a C3-C10 alicyclic hydrocarbon group, or a three- to ten-membered non-aromatic heterocyclic group {the C3-C10 alicyclic hydrocarbon group and the three- to ten-membered non-aromatic heterocyclic group may be optionally substituted with one or more substituents selected from Group E},
R¹⁰ represents a C1-C3 alkyl group,
R⁷ represents a C1-C3 alkyl group,
R¹⁵ represents a phenyl group, or a five- to six-membered aromatic heterocyclic group {the phenyl group and the five- to six-membered aromatic heterocyclic group may be optionally substituted with one or more substituents selected from Group G},
R¹⁶ represents a C1-C3 alkyl group,
R¹⁹ represents a phenyl group which is substituted at its 2-position with substituent(s) selected from Group L, a phenyl group which is substituted at its 3-position with substituent(s) selected from Group M, a phenyl group which is substituted at its 4-position with substituent(s) selected from Group N, a phenyl group which is substituted with two or more substituents selected from Group P (the two or more substituents selected from Group P may be identical to or different from each other), a pyridyl group which may be optionally with substituent(s) selected from Group P, a pyrazolyl group which may be optionally substituted with substituent(s) selected from Group G, or a pyrimidinyl group which may be optionally substituted with substituent(s) selected from Group Q.

[Embodiment 122] The present compound N wherein E represents R⁸R⁹C═N—O—CH₂—, R¹⁵—CH(R¹⁶)—O—N═C(R⁷)—, or R¹⁹O—,
R⁸ represents an indanyl group, a tetrahydronaphthyl group, a dihydrobenzofuranyl group, or a dihydrobenzopyranyl group {the indanyl group, the tetrahydronaphthyl group, the dihydrobenzofuranyl group, and the dihydrobenzopyranyl group may be optionally substituted with one or more substituents selected from Group G}, and R⁹ represents a C1-C3 alkyl group,
R⁸ and R⁹ may combine together with a nitrogen atom to which they are attached to form an indan-1-ylidene group, a 1,2,3,4-tetrahydronaphthalen-1-yline group, a 2,3-dihydrobenzofuran-3-yline group, a 3,4-dihydro-2H-1-benzofuran-4-ylidene group, a 3,4-dihydro-2H-1-benzopyran-4-ylidene group, or a 3,4-dihydro-2H-1-benzopyran-4-ylidene group {the indan-1-ylidene group, the 1,2,3,4-tetrahydronaphthalen-1-yline group, the 2,3-dihydrobenzofuran-3-yline group, the 3,4-dihydro-2H-1-benzofuran-4-ylidene group, the 3,4-dihydro-2H-1-benzopyran-4-ylidene group, and the 3,4-dihydro-2H-1-benzopyran-4-ylidene group may be optionally substituted with one or more substituents selected from Group G},
R⁷ represents a C1-C3 alkyl group,
R¹⁵ represents a phenyl group, or a five- to six-membered aromatic heterocyclic group {the phenyl group and the five- to six-membered aromatic heterocyclic group may be optionally substituted with one or more substituents selected from Group G},
R¹⁶ represents a C1-C3 alkyl group,
R¹⁹ represents a phenyl group which is substituted at its 2-position with substituent(s) selected from Group L, a phenyl group which is substituted at its 3-position with substituent(s) selected from Group M, a phenyl group which is substituted at its 4-position with substituent(s) selected from Group N, a phenyl group which is substituted with two or more substituents selected from Group P (the two or more substituents selected from Group P may be identical to or different from each other), a pyridyl group which may be optionally with substituent(s) selected from Group P, a pyrazolyl group which may be optionally substituted with substituent(s) selected from Group G, or a pyrimidinyl group which may be optionally substituted with substituent(s) selected from Group Q.

[Embodiment 123] The present compound N wherein E represents R³⁴R³⁵C═N—N═C(R¹⁰)—, R¹⁵—CH(R¹⁶)—O—N═C(R⁷)—, or R⁸R⁹C═N—O—CH₂—
R³⁴ represents a C1-C6 chain hydrocarbon group which may be optionally substituted with one or more substituents selected from Group J, a C3-C6 cycloalkyl group which may be optionally substituted with one or more halogen atoms, a phenyl group, or a five- to six-membered aromatic heterocyclic group {the phenyl group and the five- to six-membered aromatic heterocyclic group may be optionally substituted with one or more substituents selected from Group G}, and
R³⁵ represents a C1-C3 alkyl group, or a hydrogen atom,
R³⁴ and R³⁵ may combine together with a carbon atom to which they are attached to form a C3-C10 alicyclic hydrocarbon group, or a three- to ten-membered non-aromatic heterocyclic group {the C3-C10 alicyclic hydrocarbon group and the three- to ten-membered non-aromatic heterocyclic group may be optionally substituted with one or more substituents selected from Group E},
R¹⁰ represents a C1-C3 alkyl group,
R⁷ represents a C1-C3 alkyl group, R¹⁵ represents a phenyl group which may be optionally substituted with one or more substituents selected from Group G, R¹⁶ represents a C1-C3 alkyl group,
R⁸ represents an indanyl group, a tetrahydronaphthyl group, a dihydrobenzofuranyl group, or a dihydrobenzopyranyl group {the indanyl group, the tetrahydronaphthyl group, the dihydrobenzofuranyl group, and the dihydrobenzopyranyl group may be optionally substituted with one or more substituents selected from Group G}, R⁹ represents a C1-C3 alkyl group, or
R⁸ and R⁹ may combine together with a nitrogen atom to which they are attached to form an indan-1-ylidene group, a 1,2,3,4-tetrahydronaphthalen-1-yline group, a 2,3-dihydrobenzofuran-3-yline group, a 3,4-dihydro-2H-1-benzofuran-4-ylidene group, a 3,4-dihydro-2H-1-benzopyran-4-ylidene group, or a 3,4-dihydro-2H-1-benzopyran-4-ylidene group {the indan-1-ylidene group, the 1,2,3,4-tetrahydronaphthalen-1-yline group, the 2,3-dihydrobenzofuran-3-yline group, the 3,4-dihydro-2H-1-benzofuran-4-ylidene group, the 3,4-dihydro-2H-1-benzopyran-4-ylidene group, and the 3,4-dihydro-2H-1-benzopyran-4-ylidene group may be optionally substituted with one or more substituents selected from Group G}.

[Embodiment 124] The present compound N wherein E represents R¹⁵—CH(R⁶)—O—N═C(R⁷)—, or R¹⁹O—,
R⁷ represents a C1-C3 alkyl group,
R³⁵ represents a phenyl group, or a five- to six-membered aromatic heterocyclic group {the phenyl group and the five- to six-membered aromatic heterocyclic group may be optionally substituted with one or more substituents selected from Group G},
R¹⁶ represents a C1-C3 alkyl group,
R¹⁹ represents a phenyl group which is substituted at its 2-position with substituent(s) selected from Group L, a phenyl group which is substituted at its 3-position with substituent(s) selected from Group M, a phenyl group which is substituted at its 4-position with substituent(s) selected from Group N, a phenyl group which is substituted with two or more substituents selected from Group P (the two or more substituents selected from Group P may be identical to or different from each other), a pyridyl group which may be optionally with substituent(s) selected from Group P, a pyrazolyl group which may be optionally substituted with substituent(s) selected from Group G, or a pyrimidinyl group which may be optionally substituted with substituent(s) selected from Group Q.

[Embodiment 125] The present compound N wherein E represents R⁸R⁹C═N—O—CH₂—, or R¹⁵—CH(R¹⁶)—O—N═C(R⁷)—,
R⁸ represents an indanyl group, a tetrahydronaphthyl group, a dihydrobenzofuranyl group, or a dihydrobenzopyranyl group {the indanyl group, the tetrahydronaphthyl group, the dihydrobenzofuranyl group, and the dihydrobenzopyranyl group may be optionally substituted with one or more substituents selected from Group G}, and R⁹ represents a C1-C3 alkyl group,
R⁸ and R⁹ may combine together with a nitrogen atom to which they are attached to form an indan-1-ylidene group, a 1,2,3,4-tetrahydronaphthalen-1-yline group, a 2,3-dihydrobenzofuran-3-yline group, a 3,4-dihydro-2H-1-benzofuran-4-ylidene group, a 3,4-dihydro-2H-1-benzopyran-4-ylidene group, or a 3,4-dihydro-2H-1-benzopyran-4-ylidene group {the indan-1-ylidene group, the 1,2,3,4-tetrahydronaphthalen-1-yline group, the 2,3-dihydrobenzofuran-3-yline group, the 3,4-dihydro-2H-1-benzofuran-4-ylidene group, the 3,4-dihydro-2H-1-benzopyran-4-ylidene group, and the 3,4-dihydro-2H-1-benzopyran-4-ylidene group may be optionally substituted with one or more substituents selected from Group G},
R⁷ represents a C1-C3 alkyl group,
R¹⁵ represents a phenyl group, or a five- to six-membered aromatic heterocyclic group {the phenyl group and the five- to six-membered aromatic heterocyclic group may be optionally substituted with one or more substituents selected from Group G}, and
R¹⁶ represents a C1-C3 alkyl group.

[Embodiment 126] The present compound N wherein E represents R³⁴R³⁵C═N—N═C(R¹⁰)—, or R¹⁵—CH(R¹⁶)—O—N═C(R⁷)—,
R³⁴ represents a C1-C6 chain hydrocarbon group which may be optionally substituted with one or more substituents selected from Group J, a C3-C6 cycloalkyl group which may be optionally substituted with one or more halogen atoms, a phenyl group, or a five- to six-membered aromatic heterocyclic group {the phenyl group, and the five- to six-membered aromatic heterocyclic group may be optionally substituted with one or more substituents selected from Group G}, and
R³⁵ represents a C1-C3 alkyl group, or a hydrogen atom, or
R³⁴ and R³⁵ may combine together with a carbon atom to which they are attached to form a C3-C10 alicyclic hydrocarbon group, or a three- to ten-membered non-aromatic heterocyclic group {the C3-C10 alicyclic hydrocarbon group and the three- to ten-membered non-aromatic heterocyclic group may be optionally substituted with one or more substituents selected from Group E},
R¹⁰ represents a C1-C3 alkyl group,
R⁷ represents a C1-C3 alkyl group,
R¹⁵ represents a phenyl group which may be optionally substituted with one or more substituents selected from Group G, and
R¹⁶ represents a C1-C3 alkyl group.

[Embodiment 127] The present compound N wherein E represents R³⁴R³⁵C═N—N═C(R¹⁰)—, R⁸R⁹C═N—O—CH₂—, R¹⁵—CH(R¹⁶)—O—N═C(R⁷)—, or R¹⁹O—,
R³⁴ represents a phenyl group, a pyridyl group, a pyrazolyl group, or a thiazolyl group {the phenyl group, the pyridyl group, the pyrazolyl group, and the thiazolyl group may be optionally substituted with one or more substituents selected from Group G}, and R³⁵ represents a C1-C3 alkyl group,
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, a 3,4-dihydro-2H-1-benzofuran-4-ylidene group, a 3,4-dihydro-2H-1-benzopyran-4-ylidene group, or a 3,4-dihydro-2H-1-benzopyran-4-ylidene group {the indan-1-ylidene group, the 1,2,3,4-tetrahydronaphthalen-1-yline group, the 2,3-dihydrobenzofuran-3-yline group, the 3,4-dihydro-2H-1-benzofuran-4-ylidene group, the 3,4-dihydro-2H-1-benzopyran-4-ylidene group, and the 3,4-dihydro-2H-1-benzopyran-4-ylidene group may be optionally substituted with one or more substituents selected from Group G},
R¹⁰ represents a C1-C3 alkyl group,
R⁸ represents an indanyl group, a tetrahydronaphthyl group, a dihydrobenzofuranyl group, or a dihydrobenzopyranyl group {the indanyl group, the tetrahydronaphthyl group, the dihydrobenzofuranyl group, and the dihydrobenzopyranyl group may be optionally substituted with one or more substituents selected from Group J3}, R⁹ represents a C1-C3 alkyl group,
R⁷ represents a C1-C3 alkyl group,
R¹⁵ represents a phenyl group which may be optionally substituted with one or more substituents selected from Group G,
R¹⁶ represents a C1-C3 alkyl group,
R¹⁹ represents a phenyl group which is substituted at its 2- or 4-position with a halogen atom.

[Embodiment 128] The present compound N wherein E represents R³⁴R³⁵C═N—N═C(R¹⁰)—, or R¹⁹O—,
R³⁴ represents a phenyl group, a pyridyl group, a pyrazolyl group, or a thiazolyl group {the phenyl group, the pyridyl group, the pyrazolyl group, and the thiazolyl group may be optionally substituted with one or more substituents selected from Group G}, and R³⁵ represents a C1-C3 alkyl group, or
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, a 3,4-dihydro-2H-1-benzofuran-4-ylidene group, a 3,4-dihydro-2H-1-benzopyran-4-ylidene group, or a 3,4-dihydro-2H-1-benzopyran-4-ylidene group {the indan-1-ylidene group, the 1,2,3,4-tetrahydronaphthalen-1-yline group, the 2,3-dihydrobenzofuran-3-yline group, the 3,4-dihydro-2H-1-benzofuran-4-ylidene group, the 3,4-dihydro-2H-1-benzopyran-4-ylidene group, and the 3,4-dihydro-2H-1-benzopyran-4-ylidene group may be optionally substituted with one or more substituents selected from Group G},
R¹⁰ represents a C1-C3 alkyl group,
R¹⁹ represents a phenyl group which is substituted at its 2- or 4-position with a halogen atom.

[Embodiment 129] The present compound N wherein E represents R⁸R⁹C═N—O—CH₂—, or R¹⁹O—,
R⁸ represents an indanyl group, a tetrahydronaphthyl group, a dihydrobenzofuranyl group, or a dihydrobenzopyranyl group {the indanyl group, the tetrahydronaphthyl group, the dihydrobenzofuranyl group, and the dihydrobenzopyranyl group may be optionally substituted with one or more substituents selected from Group J3}, R⁹ represents a C1-C3 alkyl group, and
R¹⁹ represents a phenyl group which is substituted at its 2- or 4-position with a halogen atom.

[Embodiment 130] The present compound N wherein E represents R³⁴R³⁵C═N—N═C(R¹⁰)—, or R⁸R⁹C═N—O—CH₂—,
R³⁴ represents a phenyl group, a pyridyl group, a pyrazolyl group, or a thiazolyl group {the phenyl group, the pyridyl group, the pyrazolyl group, and the thiazolyl group may be optionally substituted with one or more substituents selected from Group G}, and R³⁵ represents a C1-C3 alkyl group, and
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, a 3,4-dihydro-2H-1-benzofuran-4-ylidene group, a 3,4-dihydro-2H-1-benzopyran-4-ylidene group, or a 3,4-dihydro-2H-1-benzopyran-4-ylidene group {the indan-1-ylidene group, the 1,2,3,4-tetrahydronaphthalen-1-yline group, the 2,3-dihydrobenzofuran-3-yline group, the 3,4-dihydro-2H-1-benzofuran-4-ylidene group, the 3,4-dihydro-2H-1-benzopyran-4-ylidene group, and the 3,4-dihydro-2H-1-benzopyran-4-ylidene group may be optionally substituted with one or more substituents selected from Group G},
R¹⁰ represents a C1-C3 alkyl group,
R⁸ represents an indanyl group, a tetrahydronaphthyl group, a dihydrobenzofuranyl group, or a dihydrobenzopyranyl group {the indanyl group, the tetrahydronaphthyl group, the dihydrobenzofuranyl group, and the dihydrobenzopyranyl group may be optionally substituted with one or more substituents selected from Group J3}, and R⁹ represents a C1-C3 alkyl group.

[Embodiment 131] The present compound N wherein E represents R³⁴R³⁵C═N—N═C(R¹⁰)—, R⁸R⁹C═N—O—CH₂—, or R¹⁹O—,
R³⁴ represents a phenyl group, a pyridyl group, a pyrazolyl group, or a thiazolyl group {the phenyl group, the pyridyl group, the pyrazolyl group, and the thiazolyl group may be optionally substituted with one or more substituents selected from Group G}, and R³⁵ represents a C1-C3 alkyl group,
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, a 3,4-dihydro-2H-1-benzofuran-4-ylidene group, a 3,4-dihydro-2H-1-benzopyran-4-ylidene group, or a 3,4-dihydro-2H-1-benzopyran-4-ylidene group {the indan-1-ylidene group, the 1,2,3,4-tetrahydronaphthalen-1-yline group, the 2,3-dihydrobenzofuran-3-yline group, and the 3,4-dihydro-2H-1-benzofuran-4-ylidene group, the 3,4-dihydro-2H-1-benzopyran-4-ylidene group, and the 3,4-dihydro-2H-1-benzopyran-4-ylidene group may be optionally substituted with one or more substituents selected from Group G},
R¹⁰ represents a C1-C3 alkyl group,
R⁸ represents an indanyl group, a tetrahydronaphthyl group, a dihydrobenzofuranyl group, or a dihydrobenzopyranyl group {the indanyl group, the tetrahydronaphthyl group, the dihydrobenzofuranyl group, and the dihydrobenzopyranyl group may be optionally substituted with one or more substituents selected from Group J3}, and R⁹ represents C1-C3 alkyl group,
R⁹ represents a phenyl group which is substituted at its 2-position with a halogen atom, a phenyl group which is substituted at its 4-position with a halogen atom, a phenyl group which is substituted with two or more halogen atoms, or a 3-chlorophenyl group.

[Embodiment 132] The present compound N wherein E represents R³⁴R³⁵C═N—N═C(R¹⁰)—, or R¹⁹O—,
R³⁴ represents a phenyl group, a pyridyl group, a pyrazolyl group, or a thiazolyl group {the phenyl group, the pyridyl group, the pyrazolyl group, and the thiazolyl group may be optionally substituted with one or more substituents selected from Group G}, and R³⁵ represents a C1-C3 alkyl group,
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, a 3,4-dihydro-2H-1-benzofuran-4-ylidene group, a 3,4-dihydro-2H-1-benzopyran-4-ylidene group, or a 3,4-dihydro-2H-1-benzopyran-4-ylidene group {the indan-1-ylidene group, the 1,2,3,4-tetrahydronaphthalen-1-yline group, the 2,3-dihydrobenzofuran-3-yline group, the 3,4-dihydro-2H-1-benzofuran-4-ylidene group, the 3,4-dihydro-2H-1-benzopyran-4-ylidene group, and the 3,4-dihydro-2H-1-benzopyran-4-ylidene group may be optionally substituted with one or more substituents selected from Group G},
R¹⁰ represents a C1-C3 alkyl group, and
R⁹ represents a phenyl group which is substituted at its 2- or 4-position with a halogen atom.

[Embodiment 133] The present compound N wherein E represents R⁸R⁹C═N—O—CH₂—, or R¹⁹O—,
R⁸ represents an indanyl group, a tetrahydronaphthyl group, a dihydrobenzofuranyl group, or a dihydrobenzopyranyl group {the indanyl group, the tetrahydronaphthyl group, the dihydrobenzofuranyl group, and the dihydrobenzopyranyl group may be optionally substituted with one or more substituents selected from Group J3}, R⁹ represents C1-C3 alkyl group,
R¹⁹ represents a phenyl group which is substituted at its 2-position with a halogen atom, a phenyl group which is substituted at its 4-position with a halogen atom, a phenyl group which is substituted with two or more halogen atoms, or a 3-chlorophenyl group.

[Embodiment 134] The present compound N wherein E represents R³⁴R³⁵C═N—N═C(R¹⁰)—, R⁸R⁹C═N—O—CH₂—, or R¹⁹O—,
R³⁴ represents a phenyl group, a pyridyl group, a pyrazolyl group, or a thiazolyl group {the phenyl group, the pyridyl group, the pyrazolyl group, and the thiazolyl group may be optionally substituted with one or more substituents selected from Group G}, and R³⁵ represents a C1-C3 alkyl group, or
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, a 3,4-dihydro-2H-1-benzofuran-4-ylidene group, a 3,4-dihydro-2H-1-benzopyran-4-ylidene group, or a 3,4-dihydro-2H-1-benzopyran-4-ylidene group {the indan-1-ylidene group, the 1,2,3,4-tetrahydronaphthalen-1-yline group, the 2,3-dihydrobenzofuran-3-yline group, the 3,4-dihydro-2H-1-benzofuran-4-ylidene group, the 3,4-dihydro-2H-1-benzopyran-4-ylidene group, and the 3,4-dihydro-2H-1-benzopyran-4-ylidene group may be optionally substituted with one or more substituents selected from Group G},
R¹⁰ represents a C1-C3 alkyl group,
R⁸ represents an indanyl group, a tetrahydronaphthyl group, a dihydrobenzofuranyl group, or a dihydrobenzopyranyl group {the indanyl group, the tetrahydronaphthyl group, the dihydrobenzofuranyl group, and the dihydrobenzopyranyl group may be optionally substituted with one or more substituents selected from Group J3}, R⁹ represents C1-C3 alkyl group, and
R¹⁹ represents a phenyl group which is substituted at its 2- or 4-position with substituent(s) selected from Group O.

[Embodiment 135] The present compound N wherein E represents R³⁴R³⁵C═N—N═C(R¹⁰)—, or R¹⁹O—,
R³⁴ represents a phenyl group, a pyridyl group, a pyrazolyl group, or a thiazolyl group {the phenyl group, the pyridyl group, the pyrazolyl group, and the thiazolyl group may be optionally substituted with one or more substituents selected from Group G}, and R³⁵ represents a C1-C3 alkyl group, or
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, a 3,4-dihydro-2H-1-benzofuran-4-ylidene group, a 3,4-dihydro-2H-1-benzopyran-4-ylidene group, or a 3,4-dihydro-2H-1-benzopyran-4-ylidene group {the indan-1-ylidene group, the 1,2,3,4-tetrahydronaphthalen-1-yline group, the 2,3-dihydrobenzofuran-3-yline group, the 3,4-dihydro-2H-1-benzofuran-4-ylidene group, the 3,4-dihydro-2H-1-benzopyran-4-ylidene group, and the 3,4-dihydro-2H-1-benzopyran-4-ylidene group may be optionally substituted with one or more substituents selected from Group G},
R¹⁰ represents a C1-C3 alkyl group, and
R⁹ represents a phenyl group which is substituted at its 2- or 4-position with substituent(s) selected from Group O.

[Embodiment 136] The present compound N wherein E represents R⁸R⁹C═N—O—CH₂—, or R¹⁹O—,
R⁸ represents an indanyl group, a tetrahydronaphthyl group, a dihydrobenzofuranyl group, or a dihydrobenzopyranyl group {the indanyl group, the tetrahydronaphthyl group, the dihydrobenzofuranyl group, and the dihydrobenzopyranyl group may be optionally substituted with one or more substituents selected from Group J3}, and R⁹ represents C1-C3 alkyl group, or
R⁹ represents a phenyl group which is substituted at its 2- or 4-position with substituent(s) selected from Group O.

[Embodiment 137] The present compound N wherein E represents R³⁴R³⁵C═N—N═C(R¹⁰)—, R⁸R⁹C═N—O—CH₂—, or R¹⁹O—,
R³⁴ represents a phenyl group, a pyridyl group, a pyrazolyl group, or a thiazolyl group {the phenyl group, the pyridyl group, the pyrazolyl group, and the thiazolyl group may be optionally substituted with one or more substituents selected from Group G}, and R³⁵ represents a C1-C3 alkyl group,
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, a 3,4-dihydro-2H-1-benzofuran-4-ylidene group, a 3,4-dihydro-2H-1-benzopyran-4-ylidene group, or a 3,4-dihydro-2H-1-benzopyran-4-ylidene group {the indan-1-ylidene group, the 1,2,3,4-tetrahydronaphthalen-1-yline group, the 2,3-dihydrobenzofuran-3-yline group, the 3,4-dihydro-2H-1-benzofuran-4-ylidene group, the 3,4-dihydro-2H-1-benzopyran-4-ylidene group, and the 3,4-dihydro-2H-1-benzopyran-4-ylidene group may be optionally substituted with one or more substituents selected from Group G},
R¹⁰ represents a C1-C3 alkyl group,
R⁸ represents an indanyl group, a tetrahydronaphthyl group, a dihydrobenzofuranyl group, or a dihydrobenzopyranyl group {the indanyl group, the tetrahydronaphthyl group, the dihydrobenzofuranyl group, and the dihydrobenzopyranyl group may be optionally substituted with one or more substituents selected from Group J3}, R⁹ represents a C1-C3 alkyl group, and
R¹⁹ represents a phenyl group which is substituted with one or more chlorine atoms.

[Embodiment 138] The present compound N wherein E represents R³⁴R³⁵C═N—N═C(R¹⁰)—, or R¹⁹O—,
R³⁴ represents a phenyl group, a pyridyl group, a pyrazolyl group, or a thiazolyl group {the phenyl group, the pyridyl group, the pyrazolyl group, and the thiazolyl group may be optionally substituted with one or more substituents selected from Group G}, and R³⁵ represents a C1-C3 alkyl group, or
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, a 3,4-dihydro-2H-1-benzofuran-4-ylidene group, a 3,4-dihydro-2H-1-benzopyran-4-ylidene group, or a 3,4-dihydro-2H-1-benzopyran-4-ylidene group {the indan-1-ylidene group, the 1,2,3,4-tetrahydronaphthalen-1-yline group, the 2,3-dihydrobenzofuran-3-yline group, and the 3,4-dihydro-2H-1-benzofuran-4-ylidene group, the 3,4-dihydro-2H-1-benzopyran-4-ylidene group, and the 3,4-dihydro-2H-1-benzopyran-4-ylidene group may be optionally substituted with one or more substituents selected from Group G},
R¹⁰ represents a C1-C3 alkyl group,
R⁹ represents a phenyl group which is substituted with one or more chlorine atoms.

[Embodiment 139] The present compound N wherein E represents R⁸R⁹C═N—O—CH₂—, or R¹⁹O—,
R⁸ represents an indanyl group, a tetrahydronaphthyl group, a dihydrobenzofuranyl group, or a dihydrobenzopyranyl group {the indanyl group, the tetrahydronaphthyl group, the dihydrobenzofuranyl group, and the dihydrobenzopyranyl group may be optionally substituted with one or more substituents selected from Group J3}, R⁹ represents a C1-C3 alkyl group, and
R¹⁹ represents a phenyl group which is substituted with one or more chlorine atoms.

[Embodiment 140] The present compound N wherein E represents R³⁴R³⁵C═N—N═C(R¹⁰)—, R⁸R⁹C═N—O—CH₂—, or R¹⁹O—,
R³⁴ represents a phenyl group which may be optionally substituted with one or more substituents selected from Group G, R³⁵ represents a C1-C3 alkyl group,
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, or a 3,4-dihydro-2H-1-benzofuran-4-ylidene group {the indan-1-ylidene group, the 1,2,3,4-tetrahydronaphthalen-1-yline group, the 2,3-dihydrobenzofuran-3-yline group, and the 3,4-dihydro-2H-1-benzofuran-4-ylidene group may be optionally substituted with one or more substituents selected from Group J3},
R¹⁰ represents a C1-C3 alkyl group,
R⁸ represents an indanyl group, a tetrahydronaphthyl group, a dihydrobenzofuranyl group, or a dihydrobenzopyranyl group {the indanyl group, the tetrahydronaphthyl group, the dihydrobenzofuranyl group, and the dihydrobenzopyranyl group may be optionally substituted with one or more substituents selected from Group J3}, R⁹ represents C1-C3 alkyl group, and
R¹⁹ represents a phenyl group which is substituted at its 2- or 4-position with a halogen atom.

[Embodiment 141] The present compound N wherein E represents R³⁴R³⁵C═N—N═C(R¹⁰)—, or R¹⁹O—,
R³⁴ represents a phenyl group which may be optionally substituted with one or more substituents selected from Group G, and R³⁵ represents a C1-C3 alkyl group,
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, or a 3,4-dihydro-2H-1-benzofuran-4-ylidene group {the indan-1-ylidene group, the 1,2,3,4-tetrahydronaphthalen-1-yline group, the 2,3-dihydrobenzofuran-3-yline group, and the 3,4-dihydro-2H-1-benzofuran-4-ylidene group may be optionally substituted with one or more substituents selected from Group J3},
R¹⁰ represents a C1-C3 alkyl group, and
R¹⁹ represents a phenyl group which is substituted at its 2- or 4-position with a halogen atom.

[Embodiment 142] The present compound N wherein E represents R³⁴R³⁵C═N—N═C(R¹⁰)—, or R⁸R^{9C}═N—O—CH₂—,
R³⁴ represents a phenyl group which may be optionally substituted with one or more substituents selected from Group G, and R³⁵ represents a C1-C3 alkyl group,
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, or a 3,4-dihydro-2H-1-benzofuran-4-ylidene group {the indan-1-ylidene group, the 1,2,3,4-tetrahydronaphthalen-1-yline group, the 2,3-dihydrobenzofuran-3-yline group, and the 3,4-dihydro-2H-1-benzofuran-4-ylidene group may be optionally substituted with one or more substituents selected from Group J3},
R¹⁰ represents a C1-C3 alkyl group,
R⁸ represents an indanyl group, a tetrahydronaphthyl group, a dihydrobenzofuranyl group, or a dihydrobenzopyranyl group {the indanyl group, the tetrahydronaphthyl group, the dihydrobenzofuranyl group, and the dihydrobenzopyranyl group may be optionally substituted with one or more substituents selected from Group J3}, and R⁹ represents a C1-C3 alkyl group.

[Embodiment 143] The present compound N wherein E represents R³⁴R³⁵C═N—N═C(R¹⁰)—, R⁸R^{9C}═N—O—CH₂—, or R¹⁹O—,
R³⁴ represents a phenyl group which may be optionally substituted with one or more substituents selected from Group G, R³⁵ represents a C1-C3 alkyl group,
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, or a 3,4-dihydro-2H-1-benzofuran-4-ylidene group {the indan-1-ylidene group, the 1,2,3,4-tetrahydronaphthalen-1-yline group, the 2,3-dihydrobenzofuran-3-yline group, and the 3,4-dihydro-2H-1-benzofuran-4-ylidene group may be optionally substituted with one or more substituents selected from Group J3},
R¹⁰ represents a C1-C3 alkyl group,
R⁸ represents an indanyl group, a tetrahydronaphthyl group, a dihydrobenzofuranyl group, or a dihydrobenzopyranyl group {the indanyl group, the tetrahydronaphthyl group, the dihydrobenzofuranyl group, and the dihydrobenzopyranyl group may be optionally substituted with one or more substituents selected from Group J31, R⁹ represents a C1-C3 alkyl group, and
R¹⁹ represents a phenyl group which is substituted at its 2-position with a halogen atom, a phenyl group which is substituted at its 4-position with a halogen atom, a phenyl group which is substituted with two or more halogen atoms, or a 3-chlorophenyl group.

[Embodiment 144] The present compound N wherein E represents R³⁴R³⁵C═N—N═C(R¹⁰)—, or R¹⁹O—,
R³⁴ represents a phenyl group which may be optionally substituted with one or more substituents selected from Group G, R³⁵ represents a C1-C3 alkyl group,
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, or a 3,4-dihydro-2H-1-benzofuran-4-ylidene group {the indan-1-ylidene group, the 1,2,3,4-tetrahydronaphthalen-1-yline group, the 2,3-dihydrobenzofuran-3-yline group, and the 3,4-dihydro-2H-1-benzofuran-4-ylidene group may be optionally substituted with one or more substituents selected from Group J3},
R¹⁰ represents a C1-C3 alkyl group, and
R¹⁹ represents a phenyl group which is substituted at its 2- or 4-position with a halogen atom.

[Embodiment 145] The present compound N wherein E represents R³⁴R³⁵C═N—N═C(R¹⁰)—, R⁸R⁹C═N—O—CH₂—, or R¹⁹O—,
R³⁴ represents a phenyl group which may be optionally substituted with one or more substituents selected from Group G, and R³⁵ represents a C1-C3 alkyl group, or
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, or a 3,4-dihydro-2H-1-benzofuran-4-ylidene group {the indan-1-ylidene group, the 1,2,3,4-tetrahydronaphthalen-1-yline group, the 2,3-dihydrobenzofuran-3-yline group, and the 3,4-dihydro-2H-1-benzofuran-4-ylidene group may be optionally substituted with one or more substituents selected from Group J3},
R¹⁰ represents a C1-C3 alkyl group,
R⁸ represents an indanyl group, a tetrahydronaphthyl group, a dihydrobenzofuranyl group, or a dihydrobenzopyranyl group {the indanyl group, the tetrahydronaphthyl group, the dihydrobenzofuranyl group, and the dihydrobenzopyranyl group may be optionally substituted with one or more substituents selected from Group J3}, R⁹ represents C1-C3 alkyl group, and
R¹⁹ represents a phenyl group which is substituted at its 2- or 4-position with substituent(s) selected from Group O.

[Embodiment 146] The present compound N wherein E represents R³⁴R³⁵C═N—N═C(R¹⁰)—, R⁸R⁹C═N—O—CH₂—, or R¹⁹O—,
R³⁴ represents a phenyl group which may be optionally substituted with one or more substituents selected from Group G, R³⁵ represents a C1-C3 alkyl group,
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, or a 3,4-dihydro-2H-1-benzofuran-4-ylidene group {the indan-1-ylidene group, the 1,2,3,4-tetrahydronaphthalen-1-yline group, the 2,3-dihydrobenzofuran-3-yline group, and the 3,4-dihydro-2H-1-benzofuran-4-ylidene group may be optionally substituted with one or more substituents selected from Group J3},
R¹⁰ represents a C1-C3 alkyl group, and
R¹⁹ represents a phenyl group which is substituted at its 2- or 4-position with substituent(s) selected from Group O.

[Embodiment 147] The present compound N wherein E represents R³⁴R³⁵C═N—N═C(R¹⁰)—, R⁸R⁹C═N—O—CH₂—, or R¹⁹O—,
R³⁴ represents a phenyl group which may be optionally substituted with one or more substituents selected from Group G, and R³⁵ represents a C1-C3 alkyl group, or
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, or a 3,4-dihydro-2H-1-benzofuran-4-ylidene group {the indan-1-ylidene group, the 1,2,3,4-tetrahydronaphthalen-1-yline group, the 2,3-dihydrobenzofuran-3-yline group, and the 3,4-dihydro-2H-1-benzofuran-4-ylidene group may be optionally substituted with one or more substituents selected from Group J3},
R¹⁰ represents a C1-C3 alkyl group,
R⁸ represents an indanyl group, a tetrahydronaphthyl group, a dihydrobenzofuranyl group, or a dihydrobenzopyranyl group {the indanyl group, the tetrahydronaphthyl group, the dihydrobenzofuranyl group, and the dihydrobenzopyranyl group may be optionally substituted with one or more substituents selected from Group J3}, R⁹ represents C1-C3 alkyl group, and
R¹⁹ represents a phenyl group which is substituted with one or more chlorine atoms.

[Embodiment 148] The present compound N wherein E represents R³⁴R³⁵C═N—N═C(R¹⁰)—, or R¹⁹O—,
R³⁴ represents a phenyl group which may be optionally substituted with one or more substituents selected from Group G, and R³⁵ represents a C1-C3 alkyl group, or
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, or a 3,4-dihydro-2H-1-benzofuran-4-ylidene group {the indan-1-ylidene group, the 1,2,3,4-tetrahydronaphthalen-1-yline group, the 2,3-dihydrobenzofuran-3-yline group, and the 3,4-dihydro-2H-1-benzofuran-4-ylidene group may be optionally substituted with one or more substituents selected from Group J3},
R¹⁰ represents a C1-C3 alkyl group, and
R¹⁹ represents a phenyl group which is substituted with one or more chlorine atoms.

[Embodiment 149] The present compound N wherein E represents R³⁴R³⁵C═N—N═C(R¹⁰)—, R⁸R⁹C═N—O—CH₂—, or R¹⁹O—,
R³⁴ represents a phenyl group which may be optionally substituted with one or more substituents selected from Group G,
R⁹, R¹⁰ and R³⁵ are identical to or different from each other and represent a C1-C3 alkyl group, or
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, or a 3,4-dihydro-2H-1-benzofuran-4-ylidene group {the indan-1-ylidene group, the 1,2,3,4-tetrahydronaphthalen-1-yline group, the 2,3-dihydrobenzofuran-3-yline group, and the 3,4-dihydro-2H-1-benzofuran-4-ylidene group may be optionally substituted with one or more substituents selected from Group J3},
R⁸ represents an indanyl group, or a tetrahydronaphthyl group {the indanyl group, and the tetrahydronaphthyl group may be optionally substituted with one or more substituents selected from Group J3}, and
R¹⁹ represents a phenyl group which is substituted at its 2-position with substituent(s) selected from Group L, a phenyl group which is substituted at its 3-position with substituent(s) selected from Group M, a phenyl group which is substituted at its 4-position with substituent(s) selected from Group N, or a phenyl group which is substituted with two or more substituents selected from Group P (the two or more substituents selected from Group P may be identical to or different from each other).

[Embodiment 150] The present compound N wherein E represents R⁸R⁹C═N—O—CH₂—, or R¹⁹O—,
R⁸ represents an indanyl group, or a tetrahydronaphthyl group {the indanyl group, and the tetrahydronaphthyl group may be optionally substituted with one or more substituents selected from Group J3},
R⁹ represents a C1-C3 alkyl group, and
R¹⁹ represents a phenyl group {the phenyl group is substituted at its 2-position with substituent(s) selected from Group L, is substituted at its 3-position with substituent(s) selected from Group M, is substituted at its 4-position with substituent(s) selected from Group N, or is substituted with two or more substituents selected from Group P},

[Embodiment 151] The present compound N wherein E represents R⁸R⁹C═N—O—CH₂—, or R¹⁹O—,
R¹⁰ and R³⁵ are identical to or different from each other and represent a C1-C3 alkyl group,
R³⁴ represents a phenyl group which may be optionally substituted with one or more substituents selected from Group G,
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, or a 3,4-dihydro-2H-1-benzofuran-4-ylidene group {the indan-1-ylidene group, the 1,2,3,4-tetrahydronaphthalen-1-yline group, the 2,3-dihydrobenzofuran-3-yline group, and the 3,4-dihydro-2H-1-benzofuran-4-ylidene group may be optionally substituted with one or more substituents selected from Group J3},
R¹⁹ represents a phenyl group which is substituted at its 2-position with substituent(s) selected from Group L, a phenyl group which is substituted at its 3-position with substituent(s) selected from Group M, a phenyl group which is substituted at its 4-position with substituent(s) selected from Group N, or a phenyl group which is substituted with two or more substituents selected from Group P (the two or more substituents selected from Group P may be identical to or different from each other).

[Embodiment 152] The present compound N wherein E represents R³⁴R³⁵C═N—N═C(R¹⁰)—, or R⁸R⁹C═N—O—CH₂—,
R³⁴ represents a phenyl group which may be optionally substituted with one or more substituents selected from Group G,
R⁹, R¹⁰, a and R³⁵ represent are identical to or different from each other and represent a C1-C3 alkyl group,
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, or a 3,4-dihydro-2H-1-benzofuran-4-ylidene group {the indan-1-ylidene group, the 1,2,3,4-tetrahydronaphthalen-1-yline group, the 2,3-dihydrobenzofuran-3-yline group, and the 3,4-dihydro-2H-1-benzofuran-4-ylidene group may be optionally substituted with one or more substituents selected from Group J3}, and
R⁸ represents an indanyl group, or a tetrahydronaphthyl group {the indanyl group, and the tetrahydronaphthyl group may be optionally substituted with one or more substituents selected from Group J3}.

[Embodiment 192] The present compound N wherein E represents R¹⁵—CR¹⁶R³⁶—O—N═C(R⁷)—, R⁸R⁹C═N—O—CH₂—, or R¹⁴O—, R⁷ and R⁸ are identical to or different from each other and represent a C1-C3 alkyl group, or a hydrogen atom, R⁸ and R¹⁵ are identical to or different from each other and represent a phenyl group which may be optionally substituted with one or more substituents selected from Group G, R¹⁴ represents a phenyl group which may be optionally substituted with one or more substituents selected from Group G, a thiazolyl group which may be optionally substituted with one or more substituents selected from Group H, or a thienyl group which may be optionally substituted with one or more halogen atoms, and R³⁶ represents a hydrogen atom.

[Embodiment 193] The present compound N wherein E represents R¹⁴O—, R¹⁴ represents a phenyl group which may be optionally substituted with one or more substituents selected from Group G, a thiazolyl group which may be optionally substituted with one or more substituents selected from Group H, or a thienyl group which may be optionally substituted with one or more halogen atoms.

[Embodiment 194] The present compound N wherein E represents R¹⁴O—, R¹⁴ represents a phenyl group which may be optionally substituted with one or more substituents selected from Group G, a thiazolyl group which may be optionally substituted with one or more substituents selected from Group H2, or a thienyl group which may be optionally substituted with one or more halogen atoms,
Group H2 is a group consisting of a C1-C3 chain hydrocarbon group, a phenyl group {the C1-C3 chain hydrocarbon group, and the phenyl group may be optionally substituted with one or more halogen atoms}, a cyclopropyl group, and a halogen atom.

[Embodiment 195] The present compound N wherein E represents R¹⁴O—, R¹⁴ represents a phenyl group, or a five- to six-membered aromatic heterocyclic group {the phenyl group, and the five- to six-membered aromatic heterocyclic group may be optionally substituted with one or more substituents selected from Group G}.

[Embodiment 196] The present compound N wherein E represents R¹⁴O—, and R¹⁴ represents a phenyl group which may be optionally substituted with one or more substituents selected from Group G.

[Embodiment 197] The present compound N wherein E represents R¹⁴O—, and R¹⁴ represents a thiazolyl group which may be optionally substituted with one or more substituents selected from Group H.

[Embodiment 198] The present compound N wherein E represents R¹⁴O—, and R¹⁴ represents a phenyl group, a 2-methyl phenyl group, a 3-methylphenyl group, a 4-methylphenyl group, a 3-fluorophenyl group, a 3-methoxyphenyl group, a 3-(trifluoromethoxy)phenyl group, a 3-phenoxyphenyl group, a 3-thienyl group, a 2-thiazolyl group, a 4-(trifluoromethyl)thizol-2-yl group, a 5-chlorothiazol-2-yl group, a 5-bromothiazol-2-yl group, a 5-(trifluoromethyl)thiazol-2-yl group, a 5-cyclopropylthiazol-2-yl group, a 5-ethynylthiazol-2-yl group, or a (5-cyclopropylethynyl)thiazol-2-yl group.

[Embodiment 199] The present compound N wherein E represents R¹⁴O—, and R¹⁴ represents a phenyl group, a 2-methyl phenyl group, a 3-methylphenyl group, a 4-methylphenyl group, a 3-fluorophenyl group, a 3-methoxyphenyl group, a 3-(trifluoromethoxy)phenyl group, or a 3-phenoxyphenyl group.

[Embodiment 200] The present compound N wherein E represents R¹⁴O—, and R¹⁴ represents a 3-thienyl group, a 2-thiazolyl group, a 4-(trifluoromethyl)thizol-2-yl group, a 5-chlorothiazol-2-yl group, a 5-bromothiazol-2-yl group, a 5-(trifluoromethyl)thiazol-2-yl group, a 5-cyclopropylthiazol-2-yl group, a 5-ethynylthiazol-2-yl group, or a (5-cyclopropylethynyl)thiazol-2-yl group.

[Embodiment 201] The present compound N wherein E represents R¹⁴O—, and R¹⁴ represents a 2-thiazolyl group, a 4-(trifluoromethyl)thizol-2-yl group, a 5-chlorothiazol-2-yl group, a 5-bromothiazol-2-yl group, a 5-(trifluoromethyl)thiazol-2-yl group, a 5-cyclopropylthiazol-2-yl group, a 5-ethynylthiazol-2-yl group, or a (5-cyclopropylethynyl)thiazol-2-yl group.

[Embodiment 202] The present compound N wherein E represents a C1-C6 alkyl group which may be optionally substituted with one or more substituents selected from Group A2, a C3-C6 cycloalkyl group which may be optionally substituted with one or more substituents selected from Group B2, R⁴R⁵C═C(R⁶)—, R¹⁵—CR¹⁶R³⁶—O—N═C(R⁷)—, R⁸R⁹C═N—O—CH₂—, R³⁴R³⁵C═N—N═C(R¹⁰)—, R¹²O—N═C(SR⁷)—S—CH₂—, or R¹⁴O—, R⁴, R⁶, and R¹⁶ represent a hydrogen atom, R⁵ represents a C1-C6 chain hydrocarbon group which may be optionally substituted with one or more substituents selected from Group A2, a C3-C6 cycloalkyl group which may be optionally substituted with one or more substituents selected from Group B2, a phenyl group, or a five- to six-membered aromatic heterocyclic group {the phenyl group, and the five- to six-membered aromatic heterocyclic group may be optionally substituted with one or more substituents selected from Group G}, R⁷, R⁹, and R¹⁰ are identical to or different from each other and represent a methyl group, or a hydrogen atom, R⁸ represents a phenyl group which may be optionally substituted with one or more substituents selected from Group G, R¹² represents a C1-C6 chain hydrocarbon group which may be optionally substituted with one or more substituents selected from Group A2, R¹⁴ represents a phenyl group, or a five- to ten-membered aromatic heterocyclic group {the phenyl group and the five- to ten-membered aromatic heterocyclic group may be optionally substituted with one or more substituents selected from Group D4}, R¹⁵ represents a phenyl group which may be optionally substituted with one or more substituents selected from Group G, and R³⁶ represents a hydrogen atom,
Group A2 is a group consisting of a C3-C6 cycloalkyl group, a halogen atom, a phenoxy group, a phenyl group, and a five- to six-membered aromatic heterocyclic group {the a phenoxy group, the phenyl group, and the five- to six-membered aromatic heterocyclic group may be optionally substituted with one or more substituents selected from Group G},
Group B2 is a group consisting of a C1-C3 alkyl group and a C3-C6 cycloalkyl group,
Group D4 is a group consisting of a C1-C6 chain hydrocarbon group which may be optionally substituted with one or more substituents selected from Group F, a C3-C6 cycloalkyl group which may be optionally substituted with one or more substituents selected from Group B, a C1-C6 alkoxy group which may be optionally substituted with one or more substituents selected from Group F, a phenoxy group, a phenyl group, a five- to six-membered aromatic heterocyclic group {the a phenoxy group, the phenyl group, and the five- to six-membered aromatic heterocyclic group may be optionally substituted with one or more substituents selected from Group G}, and a halogen atom.

[Embodiment 203] The present compound N wherein E represents a C1-C6 alkyl group which may be optionally substituted with one or more substituents selected from Group A3, a cyclopropyl group, R⁴R⁵C═C(R⁶)—, R¹⁵—CR¹⁶R³⁶—O—N═C(R⁷)—, R⁸R⁹C═N—O—CH₂—, R³⁴R³⁵C═N—N═C(R¹⁰)—, R¹²O—N═C(SR⁷)—S—CH₂—, or R¹⁴O—, R⁴, R⁶, and R³⁶ represent a hydrogen atom, R⁵ represents a C1-C6 chain hydrocarbon group which may be optionally substituted with one or more phenyl groups, a phenyl group which may be optionally substituted with one or more halogen atoms, or a cyclopropyl group, R⁷, R⁹, and R¹⁶ are identical to or different from each other and represent a methyl group, or a hydrogen atom, R⁸ represents a phenyl group which may be optionally substituted with one or more trifluoromethyl groups, R¹² represents a C1-C6 chain hydrocarbon group, R¹⁵ represents a phenyl group, R¹⁴ represents a phenyl group, a naphthyl group, a pyridyl group, a pyrimidinyl group, a thienyl group, a thiazolyl group, thiadiazolyl group, a benzothiazolyl group, a benzoxazolyl group {the phenyl group, the a naphthyl group, the pyridyl group, the pyrimidinyl group, the thienyl group, the thiazolyl group, the thiadiazolyl group, the benzothiazolyl group, and the benzoxazolyl group may be optionally substituted with one or more substituents selected from Group G}, or a pyrazolyl group {the pyrazolyl group may be optionally substituted with a phenyl group which may be optionally substituted with one or more halogen atoms},
Group A3 is a group consisting of a halogen atom, a cyclopropyl group, a phenoxy group, a phenyl group, and a pyrazolyl group {the phenyl group, and the pyrazolyl group may be optionally substituted with one or more substituents selected from Group G2},
Group G2 is a group consisting of a C1-C3 alkyl group and a halogen atom.

[Embodiment 204] The present compound N wherein E represents R⁴R⁵C═C(R⁶)—, R¹⁵—CR¹⁶R³⁶—O—N═C(R⁷)—, R⁸R⁹C═N—O—CH₂—, R³⁴R³⁵C═N—N═C(R¹⁰)—, or R¹⁴O—, R⁴, R⁶, R⁷, and R³⁶ represent a hydrogen atom, R⁵ represents a C1-C6 chain hydrocarbon group 1-C6 chain hydrocarbon group which may be optionally substituted with one or more halogen atoms, a C3-C6 cycloalkyl group which may be optionally substituted with one or more halogen atoms, or a phenyl group which may be optionally substituted with one or more substituents selected from Group G, R⁹, R¹⁶, and R³⁵ are identical to or different from each other and represent a C1-C3 alkyl group, R⁸ and R¹⁵ are identical to or different from each other and represent a phenyl group which may be optionally substituted with one or more substituents selected from Group G, R³⁴ represents a phenyl group, an indanyl group, or a five- to six-aromatic heterocyclic group {the phenyl group, the indanyl group, and the five- to six-aromatic heterocyclic group may be optionally substituted with one or more substituents selected from Group G}, R³⁴ and R³⁵ may combine together with a carbon atom to which they are attached to form an indan-1-ylidene group, or a 3,4-dihydro-2H-1-benzofuran-4-ylidene group {the indan-1-ylidene group, and the 3,4-dihydro-2H-1-benzofuran-4-ylidene group may be optionally substituted with one or more halogen atoms}, R¹⁴ represents a phenyl group, a naphthyl group, or a five- to six-membered aromatic heterocyclic group {the phenyl group, the naphthyl group, and the five- to six-membered aromatic heterocyclic group may be optionally substituted with one or more substituents selected from Group D4}.

[Embodiment 205] The present compound N wherein E represents R⁴R⁵C═C(R⁶)—, R¹⁵—CR¹⁶R³⁶—O—N═C(R⁷)—, R⁸R⁹C═N—O—CH₂—, R³⁴R³⁵C═N—N═C(R¹⁰)—, or R¹⁴O—, R⁴, R⁶, R⁷, and R³⁶ represent a hydrogen atom, R⁵ represents a C1-C6 chain hydrocarbon group which may be optionally substituted with one or more halogen atoms, a C3-C6 cycloalkyl group, or a phenyl group, R⁹, R¹⁶ and R³⁵ represent a methyl group, R⁸ and R¹⁵ are identical to or different from each other and represent a phenyl group which may be optionally substituted with one or more trifluoromethyl groups, R³⁴ represents a phenyl group, an indanyl group, a pyridyl group, or a thiazolyl group {the phenyl group, the indanyl group, the pyridyl group, and the thiazolyl group may be optionally substituted with a C1-C3 alkyl group which may be optionally substituted with one or more halogen atoms}, R³⁴ and R³⁵ may combine together with a carbon atom to which they are attached to form an indan-1-ylidene group, or a 3,4-dihydro-2H-1-benzofuran-4-ylidene group {the indan-1-ylidene group, and the 3,4-dihydro-2H-1-benzofuran-4-ylidene group may be optionally substituted with one or more halogen atoms}, R¹⁴ represents a phenyl group, a naphthyl group, a pyridyl group, a pyrimidinyl group, a thienyl group, a thiazolyl group, a thiadiazolyl group, a benzothiazolyl group, a benzoxazolyl group {the phenyl group, the naphthyl group, the pyridyl group, the pyrimidinyl group, the thienyl group, the thiazolyl group, the thiadiazolyl group, the benzothiazolyl group, and the benzoxazolyl group may be optionally substituted with one or more substituents selected from Group G}, or a pyrazolyl group {the pyrazolyl group may be optionally substituted with a phenyl group which may be optionally substituted with one or more halogen atoms}.

[Embodiment 206] The present compound N wherein E represents R¹⁴O—, and R¹⁴ represents a phenyl group which may be optionally substituted with one or more substituents selected from a halogen atom and a methyl group (with the proviso that a 2-methyl phenyl group, a 3-methylphenyl group, a 4-methylphenyl group, a 3,5-dimethylphenyl group, a 3-fluorophenyl group, a 3-bromophenyl group, and a 3-iodophenyl group are excluded).

[Embodiment 207] The present compound N wherein E represents R¹⁴O—, and R¹⁴ represents a phenyl group which is substituted with one or more chlorine atoms.

[Embodiment 208] The present compound N wherein E represents R¹⁴O—, and R¹⁴ represents a phenyl group which is substituted with one or more fluorine atoms {with the proviso that a 3-fluorophenyl group is excluded}.

[Embodiment 209] The present compound N wherein E represents R¹⁴O—, and R¹⁴ represents a phenyl group which is substituted at its 2-position with a halogen atom, a phenyl group which is substituted at its 4-position with a halogen atom, a phenyl group which is substituted with two or more halogen atoms, or a 3-chlorophenyl group.

[Embodiment 210] The present compound N wherein E represents R¹⁴O—, and R¹⁴ represents a phenyl group which is substituted at its 2-position with a halogen atom.

[Embodiment 211] The present compound N wherein E represents R¹⁴O—, and R¹⁴ represents a phenyl group which is substituted at its 4-position with a halogen atom.

[Embodiment 212] The present compound N wherein E represents R¹⁴O—, and R¹⁴ represents a phenyl group which is substituted at its 2- or 4-position with a halogen atom.

[Embodiment 213] The present compound N wherein E represents R¹⁴O—, and R¹⁴ represents a phenyl group which is substituted at its 2-position with substituent(s) selected from Group L.

[Embodiment 214] The present compound N wherein E represents R¹⁴O—, and R¹⁴ represents a phenyl group which is substituted at its 3-position with substituent(s) selected from Group M.

[Embodiment 215] The present compound N wherein E represents R¹⁴O—, and R¹⁴ represents a phenyl group which is substituted at its 4-position with substituent(s) selected from Group N.

[Embodiment 216] The present compound N wherein E represents R¹⁴O—, and R¹⁴ represents a phenyl group which is substituted at its 2- or 4-position with substituent(s) selected from Group O.

[Embodiment 217] The present compound N wherein E represents R¹⁴O—, and R¹⁴ represents a phenyl group which is substituted with two or more substituents selected from Group P.

[Embodiment 218] The present compound N wherein E represents R¹⁴O—, and R¹⁴ represents a phenyl group which is substituted at its 2-position with substituent(s) selected from Group L, a phenyl group which is substituted at its 3-position with substituent(s) selected from Group M, a phenyl group which is substituted at its 4-position with substituent(s) selected from Group N, or a phenyl group which is substituted with two or more substituents selected from Group P.

[Embodiment 219] The present compound N wherein E represents R¹⁴O—, and R¹⁴ represents a pyridyl group which may be optionally with substituent(s) selected from Group P, a pyrazolyl group which may be optionally substituted with substituent(s) selected from Group G, or a pyrimidinyl group which may be optionally substituted with substituent(s) selected from Group Q.

[Embodiment 220] The present compound N wherein E represents R¹⁴O—, and R¹⁴ represents a phenyl group which is substituted at its 2-position with substituent(s) selected from Group L, a phenyl group which is substituted at its 3-position with substituent(s) selected from Group M, a phenyl group which is substituted at its 4-position with substituent(s) selected from Group N, a phenyl group which is substituted with two or more substituents selected from Group P, a pyridyl group which may be optionally with substituent(s) selected from Group P, a pyrazolyl group which may be optionally substituted with substituent(s) selected from Group G, or a pyrimidinyl group which may be optionally substituted with substituent(s) selected from Group Q.

[Embodiment 153] The compound according to any one of [Embodiment 1] to [Embodiment 152], [Embodiment 192] to

[Embodiment 220], or the present compound N wherein R¹ represents a C1-C3 chain hydrocarbon group which may be optionally substituted with one or more halogen atoms, or a halogen atom, and n is 0.

[Embodiment 154] The compound according to any one of [Embodiment 1] to [Embodiment 152], [Embodiment 192] to [Embodiment 220], or the present compound N wherein R¹ represents a methyl group, and n is 0.

[Embodiment 155] The compound according to any one of [Embodiment 44] to [Embodiment 152], [Embodiment 192] to [Embodiment 220], or the present compound N wherein R¹ represents a C1-C3 chain hydrocarbon group which may be optionally substituted with one or more halogen atoms, or a halogen atom, n is 0, and Q represents a group represented by Q1.

[Embodiment 156] The compound according to any one of [Embodiment 44] to [Embodiment 152], [Embodiment 192] to [Embodiment 220], or the present compound N wherein R¹ represents a methyl group, n is 0, and Q represents a group represented by Q1.

[Embodiment 157] The compound according to any one of [Embodiment 44] to [Embodiment 152], [Embodiment 192] to [Embodiment 220], or the present compound N wherein R¹ represents a C1-C3 chain hydrocarbon group which may be optionally substituted with one or more halogen atoms, or a halogen atom, n is 0, and Q represents a group represented by Q2.

[Embodiment 158] The compound according to any one of [Embodiment 44] to [Embodiment 152], [Embodiment 192] to [Embodiment 220], or the present compound N wherein R¹ represents a methyl group, n is 0, and Q represents a group represented by Q2.

[Embodiment 159] The compound according to any one of [Embodiment 44] to [Embodiment 152], [Embodiment 192] to [Embodiment 220], or the present compound N wherein R¹ represents a C1-C3 chain hydrocarbon group which may be optionally substituted with one or more halogen atoms, or a halogen atom, n is 0, and Q represents a group represented by Q1 or a group represented by Q2.

[Embodiment 160] The compound according to any one of [Embodiment 44] to [Embodiment 152], [Embodiment 192] to [Embodiment 220], or the present compound N wherein R¹ represents a methyl group, n is 0, and Q represents a group represented by Q1 or a group represented by Q2.

[Embodiment 161] The compound according to any one of [Embodiment 1] to [Embodiment 152], [Embodiment 192] to [Embodiment 220], or the present compound N wherein R¹ represents a C1-C3 chain hydrocarbon group which may be optionally substituted with one or more halogen atoms, or a halogen atom, n is 0, and Q represents a group represented by Q3.

[Embodiment 162] The compound according to any one of [Embodiment 1] to [Embodiment 152], [Embodiment 192] to [Embodiment 220], or the present compound N wherein R¹ represents a methyl group, n is 0, and Q represents a group represented by Q3.

[Embodiment 163] The compound according to any one of [Embodiment 1] to [Embodiment 152], [Embodiment 192] to [Embodiment 220], or the present compound N wherein R¹ represents a C1-C3 chain hydrocarbon group which may be optionally substituted with one or more halogen atoms, or a halogen atom, n is 0, and Q represents a group represented by Q4.

[Embodiment 164] The compound according to any one of [Embodiment 1] to [Embodiment 152], [Embodiment 192] to [Embodiment 220], or the present compound N wherein R¹ represents a methyl group, n is 0, and Q represents a group represented by Q4.

[Embodiment 165] The compound according to any one of [Embodiment 1] to [Embodiment 152], [Embodiment 192] to [Embodiment 220], or the present compound N wherein R¹ represents a C1-C3 chain hydrocarbon group which may be optionally substituted with one or more halogen atoms, or a halogen atom, n is 0, and Q represents a group represented by Q5.

[Embodiment 166] The compound according to any one of [Embodiment 1] to [Embodiment 152], [Embodiment 192] to [Embodiment 220], or the present compound N wherein R¹ represents a methyl group, n is 0, and Q represents a group represented by Q5.

[Embodiment 167] The compound according to any one of [Embodiment 1] to [Embodiment 152], [Embodiment 192] to [Embodiment 220], or the present compound N wherein R¹ represents a C1-C3 chain hydrocarbon group which may be optionally substituted with one or more halogen atoms, or a halogen atom, n is 0, and Q represents a group represented by Q6.

[Embodiment 168] The compound according to any one of [Embodiment 1] to [Embodiment 152], [Embodiment 192] to [Embodiment 220], or the present compound N wherein R¹ represents a methyl group, n is 0, and Q represents a group represented by Q6.

[Embodiment 169] The compound according to any one of [Embodiment 1] to [Embodiment 152], [Embodiment 192] to [Embodiment 220], or the present compound N wherein R¹ represents a C1-C3 chain hydrocarbon group which may be optionally substituted with one or more halogen atoms, or a halogen atom, n is 0, and Q represents a group represented by Q7.

[Embodiment 170] The compound according to any one of [Embodiment 1] to [Embodiment 152], [Embodiment 192] to [Embodiment 220], or the present compound N wherein R¹ represents a methyl group, n is 0, and Q represents a group represented by Q7.

[Embodiment 171] The compound according to any one of [Embodiment 1] to [Embodiment 152], [Embodiment 192] to [Embodiment 220], or the present compound N wherein R¹ represents a C1-C3 chain hydrocarbon group which may be optionally substituted with one or more halogen atoms, or a halogen atom, n is 0, and Q represents a group represented by Q8.

[Embodiment 172] The compound according to any one of [Embodiment 1] to [Embodiment 152], [Embodiment 192] to [Embodiment 220], or the present compound N wherein R¹ represents a methyl group, n is 0, and Q represents a group represented by Q8.

[Embodiment 173] The compound according to any one of [Embodiment 1] to [Embodiment 152], [Embodiment 192] to [Embodiment 220], or the present compound N wherein R¹ represents a methyl group, n is 0, Q represents a group represented by Q8, Z¹ represents an oxygen atom, Z² represents a methyl group which is substituted with one or more fluorine atoms, Z³ represents CH, and L³ represents an oxygen atom.

[Embodiment 174] The compound according to any one of [Embodiment 1] to [Embodiment 152], [Embodiment 192] to [Embodiment 220], or the present compound N wherein R¹ represents a methyl group, n is 0, Q represents a group represented by Q8, Z¹ represents an oxygen atom, Z² represents a methyl group which is substituted with one or more fluorine atoms, Z³ represents a nitrogen atom, and L³ represents an oxygen atom.

[Embodiment 175] The compound according to any one of [Embodiment 1] to [Embodiment 152], [Embodiment 192] to [Embodiment 220], or the present compound N wherein R¹ represents a methyl group, n is 0, Q represents a group represented by Q8, Z¹ represents an oxygen atom, Z² represents a methyl group which is substituted with one or more fluorine atoms, Z³ represents a nitrogen atom, and L³ represents NH.

[Embodiment 176] The compound according to any one of [Embodiment 1] to [Embodiment 152], [Embodiment 192] to [Embodiment 220], or the present compound N wherein R¹ represents a methyl group, n is 0, Q represents a group represented by Q8, Z¹ represents CH₂, Z² represents a methyl group which is substituted with one or more fluorine atoms, Z³ represents CH, and L³ represents an oxygen atom.

[Embodiment 177] The compound according to any one of [Embodiment 1] to [Embodiment 152], [Embodiment 192] to [Embodiment 220], or the present compound N wherein R¹ represents a methyl group, n is 0, Q represents a group represented by Q8, Z¹ represents CH₂, Z² represents a methyl group which is substituted with one or more fluorine atoms, Z³ represents a nitrogen atom, and L³ represents an oxygen atom.

[Embodiment 178] The compound according to any one of [Embodiment 1] to [Embodiment 152], [Embodiment 192] to [Embodiment 220], or the present compound N wherein R¹ represents a methyl group, n is 0, Q represents a group represented by Q8, Z¹ represents CH₂, Z² represents a methyl group which is substituted with one or more fluorine atoms, Z³ represents a nitrogen atom, and L³ represents NH.

[Embodiment 179] The compound according to any one of [Embodiment 1] to [Embodiment 152], [Embodiment 192] to [Embodiment 220], or the present compound N wherein R¹ represents a hydrogen group, n is 0, Q represents a group represented by Q8, Z¹ represents NCH₃, Z² represents a methyl group which is substituted with one or more fluorine atoms, Z³ represents CH, and L³ represents NH.

[Embodiment 180] The compound according to any one of [Embodiment 1] to [Embodiment 152], [Embodiment 192] to [Embodiment 220], or the present compound N wherein R¹ represents a hydrogen atom, n is 0, Q represents a group represented by Q8, Z¹ represents NCH₃, Z² represents a methyl group which is substituted with one or more fluorine atoms, Z³ represents a nitrogen atom, and L³ represents an oxygen atom.

[Embodiment 181] The compound according to any one of [Embodiment 1] to [Embodiment 152], [Embodiment 192] to [Embodiment 220], or the present compound N wherein R¹ represents a hydrogen atom, n is 0, Q represents a group represented by Q8, Z¹ represents NCH₃, Z² represents a methyl group which is substituted with one or more fluorine atoms, Z³ represents a nitrogen atom, and L³ represents NH.

[Embodiment 182] The compound according to any one of [Embodiment 1] to [Embodiment 152], [Embodiment 192] to [Embodiment 220], or the present compound N wherein R¹ represents a methyl group, n is 0, Q represents a group represented by Q8, Z¹ represents a single bond, Z² represents a methyl group which is substituted with one or more fluorine atoms, Z³ represents CH, and L³ represents an oxygen atom.

[Embodiment 183] The compound according to any one of [Embodiment 1] to [Embodiment 152], [Embodiment 192] to [Embodiment 220], or the present compound N wherein R¹ represents a methyl group, n is 0, Q represents a group represented by Q8, Z¹ represents a single bond, Z² represents a methyl group which is substituted with one or more fluorine atoms, Z³ represents a nitrogen atom, and L³ represents an oxygen atom.

[Embodiment 184] The compound according to any one of [Embodiment 1] to [Embodiment 152], [Embodiment 192] to [Embodiment 220], or the present compound N wherein R¹ represents a methyl group, n is 0, Q represents a group represented by Q8, Z¹ represents a single bond, Z² represents a methyl group which is substituted with one or more fluorine atoms, Z³ represents a nitrogen atom, and L³ represents NH.

[Embodiment 185] The compound according to any one of [Embodiment 1] to [Embodiment 152], [Embodiment 192] to [Embodiment 220], or the present compound N wherein R¹ represents a hydrogen atom, n is 1, and R² represents a methyl group, or a halogen atom.

[Embodiment 186] The compound according to any one of [Embodiment 1] to [Embodiment 152], [Embodiment 192] to [Embodiment 220], or the present compound N wherein R¹ represents a hydrogen atom, n is 1, and R² represents a methyl group.

[Embodiment 187] The compound according to any one of [Embodiment 1] to [Embodiment 152], [Embodiment 192] to [Embodiment 220], or the present compound N wherein R¹ represents a hydrogen atom, n is 1, and R² represents a chlorine atom.

[Embodiment 188] The compound according to any one of [Embodiment 1] to [Embodiment 152], [Embodiment 192] to [Embodiment 220], or the present compound N wherein R¹ represents a hydrogen atom, n is 1, R² represents a methyl group, or a halogen atom, and Q represents a group represented by Q1.

[Embodiment 189] The compound according to any one of [Embodiment 44] to [Embodiment 152], [Embodiment 192] to [Embodiment 220], or the present compound N wherein R¹ represents a hydrogen atom, n is 1, R² represents a methyl group, and Q represents a group represented by Q1.

[Embodiment 190] The compound according to any one of [Embodiment 44] to [Embodiment 152], [Embodiment 192] to [Embodiment 220], or the present compound N wherein R¹ represents a hydrogen atom, n is 1, R² represents a hydrogen atom, and Q represents a group represented by Q1.

[Embodiment 191] The compound according to [Embodiment 116] wherein R¹ represents a methyl group, n is 0, Q represents a group represented by Q1 or a group represented by Q2, and L¹ represents an oxygen atom or NH.

Examples of the embodiment of the present compound C include the following compounds.

[Embodiment C1] The present compound C wherein E represents R³⁴R³⁵C═N—N═C(R¹⁰)—, R⁸R⁹C═N—O—CH₂—, or R¹⁹O—.

[Embodiment C2] The present compound C wherein E represents R³⁴R³⁵C═N—N═C(R¹⁰)—, or R¹⁶O—.

[Embodiment C3] The present compound C wherein E represents R⁸R⁹C═N—O—CH₂—, or R¹⁹O—,
R⁹ represents a C1-C3 alkyl group, and
R⁸ and R⁹ may combine together with a nitrogen atom to which they are attached to form an indan-1-ylidene group, a 1,2,3,4-tetrahydronaphthalen-1-yline group, a 2,3-dihydrobenzofuran-3-yline group, a 3,4-dihydro-2H-1-benzofuran-4-ylidene group, a 3,4-dihydro-2H-1-benzopyran-4-ylidene group, or a 3,4-dihydro-2H-1-benzopyran-4-ylidene group {the indan-1-ylidene group, the 1,2,3,4-tetrahydronaphthalen-1-yline group, the 2,3-dihydrobenzofuran-3-yline group, the 3,4-dihydro-2H-1-benzofuran-4-ylidene group, the 3,4-dihydro-2H-1-benzopyran-4-ylidene group, and the 3,4-dihydro-2H-1-benzopyran-4-ylidene group may be optionally substituted with one or more substituents selected from Group G}.

[Embodiment C4] The present compound C wherein E represents R³⁴R³⁵C═N—N═C(R¹⁰)—, or R⁸R⁹C═N—O—CH₂—,
R³⁴ represents a C1-C6 chain hydrocarbon group which may be optionally substituted with one or more substituents selected from Group J, a C3-C6 cycloalkyl group which may be optionally substituted with one or more halogen atoms, a phenyl group, or a five- to six-membered aromatic heterocyclic group {the phenyl group, and the five- to six-membered aromatic heterocyclic group may be optionally substituted with one or more substituents selected from Group G},
R³⁴ and R³⁵ may combine together with a carbon atom to which they are attached to form a C3-C10 alicyclic hydrocarbon group, or a three- to ten-membered non-aromatic heterocyclic group {the C3-C10 alicyclic hydrocarbon group, and the three- to ten-membered non-aromatic heterocyclic group may be optionally substituted with one or more substituents selected from Group E},
R⁹ represents a C1-C3 alkyl group, and
R⁸ and R⁹ may combine together with a nitrogen atom to which they are attached to form an indan-1-ylidene group, a 1,2,3,4-tetrahydronaphthalen-1-yline group, a 2,3-dihydrobenzofuran-3-yline group, a 3,4-dihydro-2H-1-benzofuran-4-ylidene group, a 3,4-dihydro-2H-1-benzopyran-4-ylidene group, or a 3,4-dihydro-2H-1-benzopyran-4-ylidene group {the indan-1-ylidene group, the 1,2,3,4-tetrahydronaphthalen-1-yline group, the 2,3-dihydrobenzofuran-3-yline group, the 3,4-dihydro-2H-1-benzofuran-4-ylidene group, the 3,4-dihydro-2H-1-benzopyran-4-ylidene group, and the 3,4-dihydro-2H-1-benzopyran-4-ylidene group may be optionally substituted with one or more substituents selected from Group G}.

[Embodiment C5] The present compound C wherein E represents R³⁴R³⁵C═N—N═C(R¹⁰)—, R¹⁵—CH(R¹⁶)—O—N═C(R⁷)—, or R¹⁹O—,
R³⁴ represents a C1-C6 chain hydrocarbon group which may be optionally substituted with one or more substituents selected from Group J, a C3-C6 cycloalkyl group, a phenyl group which may be optionally substituted with one or more halogen atoms, or a five- to six-membered aromatic heterocyclic group {the phenyl group, and the five- to six-membered aromatic heterocyclic group may be optionally substituted with one or more substituents selected from Group G},
R³⁴ and R³⁵ may combine together with a carbon atom to which they are attached to form a C3-C10 alicyclic hydrocarbon group, or a three- to ten-membered non-aromatic heterocyclic group {the C3-C10 alicyclic hydrocarbon group, and the three- to ten-membered non-aromatic heterocyclic group may be optionally substituted with one or more substituents selected from Group E}.

[Embodiment C6] The present compound C wherein E represents R⁸R⁹C═N—O—CH₂—, R¹⁵—CH(R¹⁶)—O—N═C(R⁷)—, or R¹⁹O—,
R⁹ represents a C1-C3 alkyl group, and
R⁸ and R⁹ may combine together with a nitrogen atom to which they are attached to form an indan-1-ylidene group, a 1,2,3,4-tetrahydronaphthalen-1-yline group, a 2,3-dihydrobenzofuran-3-yline group, a 3,4-dihydro-2H-1-benzofuran-4-ylidene group, a 3,4-dihydro-2H-1-benzopyran-4-ylidene group, or a 3,4-dihydro-2H-1-benzopyran-4-ylidene group {the indan-1-ylidene group, the 1,2,3,4-tetrahydronaphthalen-1-yline group, the 2,3-dihydrobenzofuran-3-yline group, the 3,4-dihydro-2H-1-benzofuran-4-ylidene group, the 3,4-dihydro-2H-1-benzopyran-4-ylidene group, and the 3,4-dihydro-2H-1-benzopyran-4-ylidene group may be optionally substituted with one or more substituents selected from Group G}.

[Embodiment C7] The present compound C wherein E represents R³⁴R³⁵C═N—N═C(R¹⁰)—, R¹⁵—CH(R¹⁶)—O—N═C(R⁷)—, or R⁸R⁹C═N—O—CH₂—,
R³⁴ represents a C1-C6 chain hydrocarbon group which may be optionally substituted with one or more substituents selected from Group J, a C3-C6 cycloalkyl group, a phenyl group which may be optionally substituted with one or more halogen atoms, or a five- to six-membered aromatic heterocyclic group {the phenyl group, and the five- to six-membered aromatic heterocyclic group may be optionally substituted with one or more substituents selected from Group G},
R³⁴ and R³⁵ may combine together with a carbon atom to which they are attached to form a C3-C10 alicyclic hydrocarbon group, or a three- to ten-membered non-aromatic heterocyclic group {the C3-C10 alicyclic hydrocarbon group, and the three- to ten-membered non-aromatic heterocyclic group may be optionally substituted with one or more substituents selected from Group E},
R¹⁵ represents a phenyl group which may be optionally substituted with one or more substituents selected from Group G,
R⁹ represents a C1-C3 alkyl group,
R⁸ and R⁹ may combine together with a nitrogen atom to which they are attached to form an indan-1-ylidene group, a 1,2,3,4-tetrahydronaphthalen-1-yline group, a 2,3-dihydrobenzofuran-3-yline group, a 3,4-dihydro-2H-1-benzofuran-4-ylidene group, a 3,4-dihydro-2H-1-benzopyran-4-ylidene group, or a 3,4-dihydro-2H-1-benzopyran-4-ylidene group {the indan-1-ylidene group, the 1,2,3,4-tetrahydronaphthalen-1-yline group, the 2,3-dihydrobenzofuran-3-yline group, the 3,4-dihydro-2H-1-benzofuran-4-ylidene group, the 3,4-dihydro-2H-1-benzopyran-4-ylidene group, and the 3,4-dihydro-2H-1-benzopyran-4-ylidene group may be optionally substituted with one or more substituents selected from Group G}.

[Embodiment C8] The present compound C wherein E represents R¹⁵—CH(R¹⁶)—O—N═C(R⁷)—, or R¹⁹O—.

[Embodiment C9] The present compound C wherein E represents R⁸R⁹C═N—O—CH₂—, or R¹⁵—CH(R¹⁶)—O—N═C(R⁷)—,
R⁹ represents a C1-C3 alkyl group, and
R⁸ and R⁹ may combine together with a nitrogen atom to which they are attached to form an indan-1-ylidene group, a 1,2,3,4-tetrahydronaphthalen-1-yline group, a 2,3-dihydrobenzofuran-3-yline group, a 3,4-dihydro-2H-1-benzofuran-4-ylidene group, a 3,4-dihydro-2H-1-benzopyran-4-ylidene group, or a 3,4-dihydro-2H-1-benzopyran-4-ylidene group {the indan-1-ylidene group, the 1,2,3,4-tetrahydronaphthalen-1-yline group, the 2,3-dihydrobenzofuran-3-yline group, the 3,4-dihydro-2H-1-benzofuran-4-ylidene group, the 3,4-dihydro-2H-1-benzopyran-4-ylidene group, and the 3,4-dihydro-2H-1-benzopyran-4-ylidene group may be optionally substituted with one or more substituents selected from Group G}.

[Embodiment C10] The present compound C wherein E represents R³⁴R³⁵C═N—N═C(R¹⁰)—, or R¹⁵—CH(R¹⁶)—O—N═C(R⁷)—,
R³⁴ represents a C1-C6 chain hydrocarbon group which may be optionally substituted with one or more substituents selected from Group J, a C3-C6 cycloalkyl group which may be optionally substituted with one or more halogen atoms, a phenyl group, or a five- to six-membered aromatic heterocyclic group {the phenyl group, and the five- to six-membered aromatic heterocyclic group may be optionally substituted with one or more substituents selected from Group G},
R³⁴ and R³⁵ may combine together with a carbon atom to which they are attached to form a C3-C10 alicyclic hydrocarbon group, or a three- to ten-membered non-aromatic heterocyclic group {the C3-C10 alicyclic hydrocarbon group, and the three- to ten-membered non-aromatic heterocyclic group may be optionally substituted with one or more substituents selected from Group E}, and
R¹⁵ represents a phenyl group which may be optionally substituted with one or more substituents selected from Group.

[Embodiment C11] The present compound C wherein E represents R³⁴R³⁵C═N—N═C(R¹⁰)—, R⁸R⁹C═N—O—CH₂—, R¹⁵—CH(R¹⁶)—O—N═C(R⁷)—, or R¹⁹O—,
R³⁴ represents a phenyl group, a pyridyl group, a pyrazolyl group, or a thiazolyl group {the phenyl group, the pyridyl group, the pyrazolyl group, and the thiazolyl group may be optionally substituted with one or more substituents selected from Group G}, and R³⁵ represents a C1-C3 alkyl group,
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, a 3,4-dihydro-2H-1-benzofuran-4-ylidene group, a 3,4-dihydro-2H-1-benzopyran-4-ylidene group, or a 3,4-dihydro-2H-1-benzopyran-4-ylidene group {the indan-1-ylidene group, the 1,2,3,4-tetrahydronaphthalen-1-yline group, the 2,3-dihydrobenzofuran-3-yline group, and the 3,4-dihydro-2H-1-benzofuran-4-ylidene group, the 3,4-dihydro-2H-1-benzopyran-4-ylidene group, and the 3,4-dihydro-2H-1-benzopyran-4-ylidene group may be optionally substituted with one or more substituents selected from Group G},
R⁸ represents an indanyl group, a tetrahydronaphthyl group, a dihydrobenzofuranyl group, or a dihydrobenzopyranyl group {the indanyl group, the tetrahydronaphthyl group, the dihydrobenzofuranyl group, and the dihydrobenzopyranyl group may be optionally substituted with one or more substituents selected from Group J3}, R⁹ represents a C1-C3 alkyl group,
R¹⁵ represents a phenyl group which may be optionally substituted with one or more substituents selected from Group G, and
R¹⁹ represents a phenyl group which is substituted at its 2- or 4-position with a halogen atom.

[Embodiment C12] The present compound C wherein E represents R³⁴R³⁵C═N—N═C(R¹⁰)—, or R¹⁹O—,
R³⁴ represents a phenyl group, a pyridyl group, a pyrazolyl group, or a thiazolyl group {the phenyl group, the pyridyl group, the pyrazolyl group, and the thiazolyl group may be optionally substituted with one or more substituents selected from Group G}, and R³⁵ represents a C1-C3 alkyl group,
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, a 3,4-dihydro-2H-1-benzofuran-4-ylidene group, a 3,4-dihydro-2H-1-benzopyran-4-ylidene group, or a 3,4-dihydro-2H-1-benzopyran-4-ylidene group {the indan-1-ylidene group, the 1,2,3,4-tetrahydronaphthalen-1-yline group, the 2,3-dihydrobenzofuran-3-yline group, and the 3,4-dihydro-2H-1-benzofuran-4-ylidene group, the 3,4-dihydro-2H-1-benzopyran-4-ylidene group, and the 3,4-dihydro-2H-1-benzopyran-4-ylidene group may be optionally substituted with one or more substituents selected from Group G},
R¹⁹ represents a phenyl group which is substituted at its 2- or 4-position with a halogen atom.

[Embodiment C13] The present compound C wherein E represents R⁸R⁹C═N—O—CH₂—, or R¹⁹O—,
R⁸ represents an indanyl group, a tetrahydronaphthyl group, a dihydrobenzofuranyl group, or a dihydrobenzopyranyl group {the indanyl group, the tetrahydronaphthyl group, the dihydrobenzofuranyl group, and the dihydrobenzopyranyl group may be optionally substituted with one or more substituents selected from Group J3}, R⁹ represents a C1-C3 alkyl group, and
R¹⁹ represents a phenyl group which is substituted at its 2- or 4-position with a halogen atom.

[Embodiment C14] The present compound C wherein E represents R³⁴R³⁵C═N—N═C(R¹⁰)—, or R⁸R⁹C═N—O—CH₂—,
R³⁴ represents a phenyl group, a pyridyl group, a pyrazolyl group, or a thiazolyl group {the phenyl group, the pyridyl group, the pyrazolyl group, and the thiazolyl group may be optionally substituted with one or more substituents selected from Group G}, and R³⁵ represents a C1-C3 alkyl group,
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, a 3,4-dihydro-2H-1-benzofuran-4-ylidene group, a 3,4-dihydro-2H-1-benzopyran-4-ylidene group, or a 3,4-dihydro-2H-1-benzopyran-4-ylidene group {the indan-1-ylidene group, the 1,2,3,4-tetrahydronaphthalen-1-yline group, the 2,3-dihydrobenzofuran-3-yline group, and the 3,4-dihydro-2H-1-benzofuran-4-ylidene group, the 3,4-dihydro-2H-1-benzopyran-4-ylidene group, and the 3,4-dihydro-2H-1-benzopyran-4-ylidene group may be optionally substituted with one or more substituents selected from Group G},
R⁸ represents an indanyl group, a tetrahydronaphthyl group, a dihydrobenzofuranyl group, or a dihydrobenzopyranyl group {the indanyl group, the tetrahydronaphthyl group, the dihydrobenzofuranyl group, and the dihydrobenzopyranyl group may be optionally substituted with one or more substituents selected from Group J3}, and R⁹ represents a C1-C3 alkyl group.

[Embodiment C15] The present compound C wherein E represents R³⁴R³⁵C═N—N═C(R¹⁰)—, R⁸R⁹C═N—O—CH₂—, or R¹⁹O—,
R³⁴ represents a phenyl group, a pyridyl group, a pyrazolyl group, or a thiazolyl group {the phenyl group, the pyridyl group, the pyrazolyl group, and the thiazolyl group may be optionally substituted with one or more substituents selected from Group G}, and R³⁵ represents a C1-C3 alkyl group,
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, a 3,4-dihydro-2H-1-benzofuran-4-ylidene group, a 3,4-dihydro-2H-1-benzopyran-4-ylidene group, or a 3,4-dihydro-2H-1-benzopyran-4-ylidene group {the indan-1-ylidene group, the 1,2,3,4-tetrahydronaphthalen-1-yline group, the 2,3-dihydrobenzofuran-3-yline group, the 3,4-dihydro-2H-1-benzofuran-4-ylidene group, the 3,4-dihydro-2H-1-benzopyran-4-ylidene group, and the 3,4-dihydro-2H-1-benzopyran-4-ylidene group may be optionally substituted with one or more substituents selected from Group G},
R⁸ represents an indanyl group, a tetrahydronaphthyl group, a dihydrobenzofuranyl group, or a dihydrobenzopyranyl group {the indanyl group, the tetrahydronaphthyl group, the dihydrobenzofuranyl group, and the dihydrobenzopyranyl group may be optionally substituted with one or more substituents selected from Group J31, R⁹ represents a C1-C3 alkyl group, and
R¹⁹ represents a phenyl group which is substituted at its 2-position with a halogen atom, a phenyl group which is substituted at its 4-position with a halogen atom, a phenyl group which is substituted with two or more halogen atoms, or a 3-chlorophenyl group.

[Embodiment C16] The present compound C wherein E represents R³⁴R³⁵C═N—N═C(R¹⁰)—, or R¹⁹O—,
R³⁴ represents a phenyl group, a pyridyl group, a pyrazolyl group, or a thiazolyl group {the phenyl group, the pyridyl group, the pyrazolyl group, and the thiazolyl group may be optionally substituted with one or more substituents selected from Group G}, and R³⁵ represents a C1-C3 alkyl group,
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, a 3,4-dihydro-2H-1-benzofuran-4-ylidene group, a 3,4-dihydro-2H-1-benzopyran-4-ylidene group, or a 3,4-dihydro-2H-1-benzopyran-4-ylidene group {the indan-1-ylidene group, the 1,2,3,4-tetrahydronaphthalen-1-yline group, the 2,3-dihydrobenzofuran-3-yline group, the 3,4-dihydro-2H-1-benzofuran-4-ylidene group, the 3,4-dihydro-2H-1-benzopyran-4-ylidene group, and the 3,4-dihydro-2H-1-benzopyran-4-ylidene group may be optionally substituted with one or more substituents selected from Group G}, and
R¹⁹ represents a phenyl group which is substituted at its 2- or 4-position with a halogen atom.

[Embodiment C17] The present compound C wherein E represents R⁸R⁹C═N—O—CH₂—, or R¹⁹O—,
R⁸ represents an indanyl group, a tetrahydronaphthyl group, a dihydrobenzofuranyl group, or a dihydrobenzopyranyl group {the indanyl group, the tetrahydronaphthyl group, the dihydrobenzofuranyl group, and the dihydrobenzopyranyl group may be optionally substituted with one or more substituents selected from Group J3}, R⁹ represents a C1-C3 alkyl group,
R¹⁹ represents a phenyl group which is substituted at its 2-position with substituent(s), a phenyl group which is substituted at its 4-position with a halogen atom, a phenyl group which is substituted with two or more halogen atoms, or a 3-chlorophenyl group.

[Embodiment C18] The present compound C wherein E represents R³⁴R³⁵C═N—N═C(R¹⁰)—, R⁸R⁹C═N—O—CH₂—, or R¹⁹O—,
R³⁴ represents a phenyl group, a pyridyl group, a pyrazolyl group, or a thiazolyl group {the phenyl group, the pyridyl group, the pyrazolyl group, and the thiazolyl group may be optionally substituted with one or more substituents selected from Group G}, and R³⁵ represents a C1-C3 alkyl group,
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, a 3,4-dihydro-2H-1-benzofuran-4-ylidene group, a 3,4-dihydro-2H-1-benzopyran-4-ylidene group, or a 3,4-dihydro-2H-1-benzopyran-4-ylidene group {the indan-1-ylidene group, the 1,2,3,4-tetrahydronaphthalen-1-yline group, the 2,3-dihydrobenzofuran-3-yline group, the 3,4-dihydro-2H-1-benzofuran-4-ylidene group, the 3,4-dihydro-2H-1-benzopyran-4-ylidene group, and the 3,4-dihydro-2H-1-benzopyran-4-ylidene group may be optionally substituted with one or more substituents selected from Group G},
R⁸ represents an indanyl group, a tetrahydronaphthyl group, a dihydrobenzofuranyl group, or a dihydrobenzopyranyl group {the indanyl group, the tetrahydronaphthyl group, the dihydrobenzofuranyl group, and the dihydrobenzopyranyl group may be optionally substituted with one or more substituents selected from Group J3}, R⁹ represents a C1-C3 alkyl group, and
R¹⁹ represents a phenyl group which is substituted at its 2- or 4-position with substituent(s) selected from Group O.

[Embodiment C19] The present compound C wherein E represents R³⁴R³⁵C═N—N═C(R¹⁰)—, or R¹⁹O—,
R³⁴ represents a phenyl group, a pyridyl group, a pyrazolyl group, or a thiazolyl group {the phenyl group, the pyridyl group, the pyrazolyl group, and the thiazolyl group may be optionally substituted with one or more substituents selected from Group G}, and R³⁵ represents a C1-C3 alkyl group,
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, a 3,4-dihydro-2H-1-benzofuran-4-ylidene group, a 3,4-dihydro-2H-1-benzopyran-4-ylidene group, or a 3,4-dihydro-2H-1-benzopyran-4-ylidene group {the indan-1-ylidene group, the 1,2,3,4-tetrahydronaphthalen-1-yline group, the 2,3-dihydrobenzofuran-3-yline group, the 3,4-dihydro-2H-1-benzofuran-4-ylidene group, the 3,4-dihydro-2H-1-benzopyran-4-ylidene group, and the 3,4-dihydro-2H-1-benzopyran-4-ylidene group may be optionally substituted with one or more substituents selected from Group G}, and
R¹⁹ represents a phenyl group which is substituted at its 2- or 4-position with substituent(s) selected from Group O.

[Embodiment C20] The present compound C wherein E represents R⁸R⁹C═N—O—CH₂—, or R¹⁹O—,
R⁸ represents an indanyl group, a tetrahydronaphthyl group, a dihydrobenzofuranyl group, or a dihydrobenzopyranyl group {the indanyl group, the tetrahydronaphthyl group, the dihydrobenzofuranyl group, and the dihydrobenzopyranyl group may be optionally substituted with one or more substituents selected from Group J3}, R⁹ represents a C1-C3 alkyl group, and
R¹⁹ represents a phenyl group which is substituted at its 2- or 4-position with substituent(s) selected from Group O.

[Embodiment C21] The present compound C wherein E represents R³⁴R³⁵C═N—N═C(R¹⁰)—, R⁸R⁹C═N—O—CH₂—, or R¹⁹O—,
R³⁴ represents a phenyl group, a pyridyl group, a pyrazolyl group, or a thiazolyl group {the phenyl group, the pyridyl group, the pyrazolyl group, and the thiazolyl group may be optionally substituted with one or more substituents selected from Group G}, and R³⁵ represents a C1-C3 alkyl group,
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, a 3,4-dihydro-2H-1-benzofuran-4-ylidene group, a 3,4-dihydro-2H-1-benzopyran-4-ylidene group, or a 3,4-dihydro-2H-1-benzopyran-4-ylidene group {the indan-1-ylidene group, the 1,2,3,4-tetrahydronaphthalen-1-yline group, the 2,3-dihydrobenzofuran-3-yline group, the 3,4-dihydro-2H-1-benzofuran-4-ylidene group, the 3,4-dihydro-2H-1-benzopyran-4-ylidene group, and the 3,4-dihydro-2H-1-benzopyran-4-ylidene group may be optionally substituted with one or more substituents selected from Group G},
R⁸ represents an indanyl group, a tetrahydronaphthyl group, a dihydrobenzofuranyl group, or a dihydrobenzopyranyl group {the indanyl group, the tetrahydronaphthyl group, the dihydrobenzofuranyl group, and the dihydrobenzopyranyl group may be optionally substituted with one or more substituents selected from Group J3}, R⁹ represents a C1-C3 alkyl group, and
R¹⁹ represents a phenyl group which is substituted with one or more chlorine atoms.

[Embodiment C22] The present compound C wherein E represents R³⁴R³⁵C═N—N═C(R¹⁰)—, or R¹⁹O—,
R³⁴ represents a phenyl group, a pyridyl group, a pyrazolyl group, or a thiazolyl group {the phenyl group, the pyridyl group, the pyrazolyl group, and the thiazolyl group may be optionally substituted with one or more substituents selected from Group G}, and R³⁵ represents a C1-C3 alkyl group,
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, a 3,4-dihydro-2H-1-benzofuran-4-ylidene group, a 3,4-dihydro-2H-1-benzopyran-4-ylidene group, or a 3,4-dihydro-2H-1-benzopyran-4-ylidene group {the indan-1-ylidene group, the 1,2,3,4-tetrahydronaphthalen-1-yline group, the 2,3-dihydrobenzofuran-3-yline group, and the 3,4-dihydro-2H-1-benzofuran-4-ylidene group, the 3,4-dihydro-2H-1-benzopyran-4-ylidene group, and the 3,4-dihydro-2H-1-benzopyran-4-ylidene group may be optionally substituted with one or more substituents selected from Group G}, and
R¹⁹ represents a phenyl group which is substituted with one or more chlorine atoms.

[Embodiment C23] The present compound C wherein E represents R⁸R⁹C═N—O—CH₂—, or R¹⁹O—,
R⁸ represents an indanyl group, a tetrahydronaphthyl group, a dihydrobenzofuranyl group, or a dihydrobenzopyranyl group {the indanyl group, the tetrahydronaphthyl group, the dihydrobenzofuranyl group, and the dihydrobenzopyranyl group may be optionally substituted with one or more substituents selected from Group J3}, R⁹ represents a C1-C3 alkyl group, and
R¹⁹ represents a phenyl group which is substituted with one or more chlorine atoms.

[Embodiment C24] The present compound C wherein E represents R³⁴R³⁵C═N—N═C(R¹⁰)—, R⁸R⁹C═N—O—CH₂—, or R¹⁹O—,
R³⁴ represents a phenyl group which may be optionally substituted with one or more substituents selected from Group G, R³⁵ represents a C1-C3 alkyl group,
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, or a 3,4-dihydro-2H-1-benzofuran-4-ylidene group {the indan-1-ylidene group, the 1,2,3,4-tetrahydronaphthalen-1-yline group, the 2,3-dihydrobenzofuran-3-yline group, and the 3,4-dihydro-2H-1-benzofuran-4-ylidene group may be optionally substituted with one or more substituents selected from Group J3},
R⁸ represents an indanyl group, a tetrahydronaphthyl group, a dihydrobenzofuranyl group, or a dihydrobenzopyranyl group {the indanyl group, the tetrahydronaphthyl group, the dihydrobenzofuranyl group, and the dihydrobenzopyranyl group may be optionally substituted with one or more substituents selected from Group J3}, R⁹ represents C1-C3 alkyl group, and
R¹⁹ represents a phenyl group which is substituted at its 2- or 4-position with a halogen atom.

[Embodiment C25] The present compound C wherein E represents R³⁴R³⁵C═N—N═C(R¹⁰)—, or R¹⁹O—,
R³⁴ represents a phenyl group which may be optionally substituted with one or more substituents selected from Group G, R³⁵ represents a C1-C3 alkyl group, or
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, or a 3,4-dihydro-2H-1-benzofuran-4-ylidene group {the indan-1-ylidene group, the 1,2,3,4-tetrahydronaphthalen-1-yline group, the 2,3-dihydrobenzofuran-3-yline group, and the 3,4-dihydro-2H-1-benzofuran-4-ylidene group may be optionally substituted with one or more substituents selected from Group J3}, and
R¹⁹ represents a phenyl group which is substituted at its 2- or 4-position with a halogen atom.

[Embodiment C26] The present compound C wherein E represents R³⁴R³⁵C═N—N═C(R¹⁰)—, or R⁸R⁹C═N—O—CH₂—,
R³⁴ represents a phenyl group which may be optionally substituted with one or more substituents selected from Group G, R³⁵ represents a C1-C3 alkyl group,
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, or a 3,4-dihydro-2H-1-benzofuran-4-ylidene group {the indan-1-ylidene group, the 1,2,3,4-tetrahydronaphthalen-1-yline group, the 2,3-dihydrobenzofuran-3-yline group, and the 3,4-dihydro-2H-1-benzofuran-4-ylidene group may be optionally substituted with one or more substituents selected from Group J3},
R⁸ represents an indanyl group, a tetrahydronaphthyl group, a dihydrobenzofuranyl group, or a dihydrobenzopyranyl group {the indanyl group, the tetrahydronaphthyl group, the dihydrobenzofuranyl group, and the dihydrobenzopyranyl group may be optionally substituted with one or more substituents selected from Group J3}, and R⁹ represents a C1-C3 alkyl group.

[Embodiment C27] The present compound C wherein E represents R³⁴R³⁵C═N—N═C(R¹⁰)—, R⁸R⁹C═N—O—CH₂—, or R¹⁹O—,
R³⁴ represents a phenyl group which may be optionally substituted with one or more substituents selected from Group G, R³⁵ represents a C1-C3 alkyl group,
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, or a 3,4-dihydro-2H-1-benzofuran-4-ylidene group {the indan-1-ylidene group, the 1,2,3,4-tetrahydronaphthalen-1-yline group, the 2,3-dihydrobenzofuran-3-yline group, and the 3,4-dihydro-2H-1-benzofuran-4-ylidene group may be optionally substituted with one or more substituents selected from Group J3},
R⁸ represents an indanyl group, a tetrahydronaphthyl group, a dihydrobenzofuranyl group, or a dihydrobenzopyranyl group {the indanyl group, the tetrahydronaphthyl group, the dihydrobenzofuranyl group, and the dihydrobenzopyranyl group may be optionally substituted with one or more substituents selected from Group J3}, R⁹ represents a C1-C3 alkyl group, and
R¹⁹ represents a phenyl group which is substituted at its 2-position with a halogen atom, a phenyl group which is substituted at its 4-position with a halogen atom, a phenyl group which is substituted with two or more halogen atoms, or a 3-chlorophenyl group.

[Embodiment C28] The present compound C wherein E represents R³⁴R³⁵C═N—N═C(R¹⁰)—, R⁸R⁹C═N—O—CH₂—, or R¹⁹O—,
R³⁴ represents a phenyl group which may be optionally substituted with one or more substituents selected from Group G, R³⁵ represents a C1-C3 alkyl group,
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, or a 3,4-dihydro-2H-1-benzofuran-4-ylidene group {the indan-1-ylidene group, the 1,2,3,4-tetrahydronaphthalen-1-yline group, the 2,3-dihydrobenzofuran-3-yline group, and the 3,4-dihydro-2H-1-benzofuran-4-ylidene group may be optionally substituted with one or more substituents selected from Group J3}, and
R¹⁹ represents a phenyl group which is substituted at its 2- or 4-position with a halogen atom.

[Embodiment C29] The present compound C wherein E represents R³⁴R³⁵C═N—N═C(R¹⁰)—, R⁸R⁹C═N—O—CH₂—, or R¹⁹O—,
R³⁴ represents a phenyl group which may be optionally substituted with one or more substituents selected from Group G, and R³⁵ represents a C1-C3 alkyl group, or
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, or a 3,4-dihydro-2H-1-benzofuran-4-ylidene group {the indan-1-ylidene group, the 1,2,3,4-tetrahydronaphthalen-1-yline group, the 2,3-dihydrobenzofuran-3-yline group, and the 3,4-dihydro-2H-1-benzofuran-4-ylidene group may be optionally substituted with one or more substituents selected from Group J3},
R⁸ represents an indanyl group, a tetrahydronaphthyl group, a dihydrobenzofuranyl group, or a dihydrobenzopyranyl group {the indanyl group, the tetrahydronaphthyl group, the dihydrobenzofuranyl group, and the dihydrobenzopyranyl group may be optionally substituted with one or more substituents selected from Group J3}, R⁹ represents a C1-C3 alkyl group, and
R¹⁹ represents a phenyl group which is substituted at its 2- or 4-position with substituent(s) selected from Group O.

[Embodiment C30] The present compound C wherein E represents R³⁴R³⁵C═N—N═C(R¹⁰)—, or R¹⁹O—,
R³⁴ represents a phenyl group which may be optionally substituted with one or more substituents selected from Group G, R³⁵ represents a C1-C3 alkyl group,
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, or a 3,4-dihydro-2H-1-benzofuran-4-ylidene group {the indan-1-ylidene group, the 1,2,3,4-tetrahydronaphthalen-1-yline group, the 2,3-dihydrobenzofuran-3-yline group, and the 3,4-dihydro-2H-1-benzofuran-4-ylidene group may be optionally substituted with one or more substituents selected from Group J3}, and
R¹⁹ represents a phenyl group which is substituted at its 2- or 4-position with substituent(s) selected from Group O.

[Embodiment C31] The present compound C wherein E represents R³⁴R³⁵C═N—N═C(R¹⁰)—, R⁸R⁹C═N—O—CH₂—, or R¹⁹O—,
R³⁴ represents a phenyl group which may be optionally substituted with one or more substituents selected from Group G, and R³⁵ represents a C1-C3 alkyl group, or
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, or a 3,4-dihydro-2H-1-benzofuran-4-ylidene group {the indan-1-ylidene group, the 1,2,3,4-tetrahydronaphthalen-1-yline group, the 2,3-dihydrobenzofuran-3-yline group, and the 3,4-dihydro-2H-1-benzofuran-4-ylidene group may be optionally substituted with one or more substituents selected from Group J3},
R⁸ represents an indanyl group, a tetrahydronaphthyl group, a dihydrobenzofuranyl group, or a dihydrobenzopyranyl group {the indanyl group, the tetrahydronaphthyl group, the dihydrobenzofuranyl group, and the dihydrobenzopyranyl group may be optionally substituted with one or more substituents selected from Group J3}, R⁹ represents a C1-C3 alkyl group, and
R¹⁹ represents a phenyl group which is substituted with one or more chlorine atoms.

[Embodiment C32] The present compound C wherein E represents R³⁴R³⁵C═N—N═C(R¹⁰)—, or R¹⁹O—,
R³⁴ represents a phenyl group which may be optionally substituted with one or more substituents selected from Group G, and R³⁵ represents a C1-C3 alkyl group, or
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, or a 3,4-dihydro-2H-1-benzofuran-4-ylidene group {the indan-1-ylidene group, the 1,2,3,4-tetrahydronaphthalen-1-yline group, the 2,3-dihydrobenzofuran-3-yline group, and the 3,4-dihydro-2H-1-benzofuran-4-ylidene group may be optionally substituted with one or more substituents selected from Group J3}, and
R¹⁹ represents a phenyl group which is substituted with one or more chlorine atoms.

[Embodiment C33] The present compound C wherein E represents R³⁴R³⁵C═N—N═C(R¹⁰)—, R⁸R⁹C═N—O—CH²—, or R¹⁹O—,
R³⁴ represents a phenyl group which may be optionally substituted with one or more substituents selected from Group G,
R⁹ and R³⁵ are identical to or different from each other and represent, C1-C3 alkyl group,
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, or a 3,4-dihydro-2H-1-benzofuran-4-ylidene group {the indan-1-ylidene group, the 1,2,3,4-tetrahydronaphthalen-1-yline group, the 2,3-dihydrobenzofuran-3-yline group, and the 3,4-dihydro-2H-1-benzofuran-4-ylidene group may be optionally substituted with one or more substituents selected from Group J3},
R⁸ represents an indanyl group, or a tetrahydronaphthyl group {the indanyl group, and the tetrahydronaphthyl group may be optionally substituted with one or more substituents selected from Group J3}, and
R¹⁹ represents a phenyl group which is substituted at its 2-position with substituent(s) selected from Group L a phenyl group which is substituted at its 3-position with substituent(s) selected from Group M, a phenyl group which is substituted at its 4-position with substituent(s) selected from Group N, or a phenyl group which is substituted with two or more substituents selected from Group P (the two or more substituents selected from Group P may be identical to or different from each other).

[Embodiment C34] The present compound C wherein E represents R⁸R⁹C═N—O—CH²—, or R¹⁹O—,
R⁸ represents an indanyl group, or a tetrahydronaphthyl group {the indanyl group, and the tetrahydronaphthyl group may be optionally substituted with one or more substituents selected from Group J3},
R⁹ represents a C1-C3 alkyl group, and
R¹⁹ represents a phenyl group {the phenyl group is substituted at its 2-position with substituent(s) selected from Group L, is substituted at its 3-position with substituent(s) selected from Group M, is substituted at its 4-position with substituent(s) selected from Group N, or is substituted with two or more substituent(s) selected from Group P.

[Embodiment C35] The present compound C wherein E represents R³⁴R³⁵C═N—N═C(R¹⁰)—, R⁸R⁹C═N—O—CH²—, or R¹⁹O—,
R³⁵ represents a C1-C3 alkyl group, and
R³⁴ represents a phenyl group which may be optionally substituted with one or more substituents selected from Group G, or
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, or a 3,4-dihydro-2H-1-benzofuran-4-ylidene group {the indan-1-ylidene group, the 1,2,3,4-tetrahydronaphthalen-1-yline group, the 2,3-dihydrobenzofuran-3-yline group, and the 3,4-dihydro-2H-1-benzofuran-4-ylidene group may be optionally substituted with one or more substituents selected from Group J3}, and
R¹⁹ represents a phenyl group which is substituted at its 2-position with substituent(s) selected from Group L, a phenyl group which is substituted at its 3-position with substituent(s) selected from Group M, a phenyl group which is substituted at its 4-position with substituent(s) selected from Group N, or a phenyl group which is substituted with two or more substituents selected from Group P (the two or more substituents selected from Group P may be identical to or different from each other).

[Embodiment C36] The present compound C wherein E represents R³⁴R³⁵C═N—N═C(R¹⁰)—, or R⁸R⁹C═N—O—CH²—,
R³⁴ represents a phenyl group which may be optionally substituted with one or more substituents selected from Group G,
R⁹ and R³⁵ are identical to or different from each other and represent a C1-C3 alkyl group,
R³⁴ and R³⁵ may combine together with a carbon atom to which they are attached to form an indan-1-ylidene group, a 1,2,3,4-tetrahydronaphthalen-1-yline group, a 2,3-dihydrobenzofuran-3-yline group, or a 3,4-dihydro-2H-1-benzofuran-4-ylidene group {the indan-1-ylidene group, the 1,2,3,4-tetrahydronaphthalen-1-yline group, the 2,3-dihydrobenzofuran-3-yline group, and the 3,4-dihydro-2H-1-benzofuran-4-ylidene group may be optionally substituted with one or more substituents selected from Group J3}, and
R⁸ represents an indanyl group, or a tetrahydronaphthyl group {the indanyl group, and the tetrahydronaphthyl group may be optionally substituted with one or more substituents selected from Group J3}.

[Embodiment C37] The present compound C wherein E represents R¹⁹O—, R¹⁹ represents a phenyl group which is substituted at its 2-position with a halogen atom, a phenyl group which is substituted at its 4-position with a halogen atom, a 3-chlorophenyl group, a phenyl group which is substituted with two or more halogen atoms, or a pyridyl group which may be optionally substituted with one or more halogen atoms.

[Embodiment C38] The present compound C wherein E represents R¹⁹O—, R¹⁹ represents a 2-fluorophenyl group, a 4-fluorophenyl group, a 3-chlorophenyl group, a phenyl group which is substituted with two or more fluorine atoms, or a pyridyl group which may be optionally substituted with one or more fluorine atoms.

[Embodiment C39] The compound according to any one of [Embodiment C1] to [Embodiment C38] or the present compound C wherein Q represents a group represented by Q1.

[Embodiment C40] The compound according to any one of [Embodiment C1] to [Embodiment C38] or the present compound C wherein Q represents a group represented by Q2.

[Embodiment C41] A compound represented by formula (IIa):

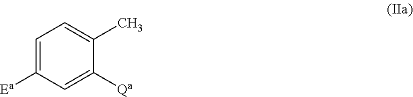

[wherein
Q^{a} represents a group represented by Q1, or a group represented by Q2,
E^{a} represents R^{34a}R^{35a}C═N—N═C(R^{10a})—, R^{8a}R^{9a}C═N—O—CH₂—, R^{15a}—CH(R^{16a})—O—N═C(R^{7a})—, or R^{14a}O—,
R^{34a} represents a C1-C6 chain hydrocarbon group which may be optionally substituted with one or more substituents selected from Group J, a C3-C10 alicyclic hydrocarbon group which may be optionally substituted with one or more substituents selected from Group E, a phenyl group, or a five- to six-membered aromatic heterocyclic group {the phenyl group, and the five- to six-membered aromatic heterocyclic group may be optionally substituted with one or more substituents selected from Group G},
R^{9a} and R^{35a} are identical to or different from each other and represent a C1-C3 alkyl group, or a hydrogen atom,
R^{7a}, R^{10a}, and R^{16a} are identical to or different from each other and represent a C1-C3 alkyl group,
R^{8a} represents an indanyl group, a tetrahydronaphthyl group, a dihydrobenzofuranyl group, or a dihydrobenzopyranyl group {the indanyl group, the tetrahydronaphthyl group, the dihydrobenzofuranyl group, and the dihydrobenzopyranyl group may be optionally substituted with one or more substituents selected from Group G},
R^{8a} and R^{9a} may combine together a carbon atom to which they are attached to form a C3-C10 alicyclic hydrocarbon group, or a three- to ten-membered non-aromatic heterocyclic group {the C3-C10 alicyclic hydrocarbon group, and the three- to ten-membered non-aromatic heterocyclic group may be optionally substituted with one or more substituents selected from Group E},
R^{34a} and R^{35a} may combine together with a carbon atom to which they are attached to form a C3-C10 alicyclic hydrocarbon group, or a three- to ten-membered non-aromatic heterocyclic group {the C3-C10 alicyclic hydrocarbon group, and the three- to ten-membered non-aromatic heterocyclic group may be optionally substituted with one or more substituents selected from Group E},
R^{15a} represents a phenyl group, or a five- to six-membered aromatic heterocyclic group {the phenyl group, and the five- to six-membered aromatic heterocyclic group may be optionally substituted with one or more substituents selected from Group G},
R^{14a} represents a phenyl group which is substituted at its 2-position with substituent(s) selected from Group L, a phenyl group which is substituted at its 3-position with substituent(s) selected from Group M, a phenyl group which is substituted at its 4-position with substituent(s) selected from Group N, a phenyl group which is substituted with two or more substituents selected from Group P (the two or more substituents selected from Group P may be identical to or different from each other), a pyridyl group which may be optionally with substituent(s) selected from Group P, a pyrazolyl group which may be optionally substituted with substituent(s) selected from Group G, or a pyrimidinyl group which may be optionally substituted with substituent(s) selected from Group Q.]
or its N oxide or agriculturally acceptable salt thereof.

[Embodiment C42] The compound according to [Embodiment C41] wherein E^{a} represents R^{14a}O—, R^{14a} represents a phenyl group which is substituted at its 2-position with substituent(s) selected from Group V, a phenyl group which is substituted at its 4-position with substituent(s) selected from Group V, a 3-chlorophenyl group, a phenyl group which is substituted with two or more halogen atoms, or a pyridyl group which may be optionally substituted with one or more halogen atoms.

[Embodiment C43] The compound according to [Embodiment C41] wherein E^{a} represents R^{34a}R^{35a}C═N—N═C(R^{10a})—, or R^{14a}O—.

[Embodiment C44] The compound according to [Embodiment C41] wherein E^{a} represents R^{15a}—CH(R^{16a})—O—N═C(R^{7a})—, or R^{14a}O—.

[Embodiment C45] The compound according to [Embodiment C41] wherein E^{a} represents R^{34a}R^{35a}C═N—N═C(R^{10a})—, R^{15a}—CH(R^{6a})—O—N═C(R^{7a})—, or R^{14a}O—.

[Embodiment C46] The compound according to [Embodiment C41] wherein E^{a} represents R^{14a}O—, and R^{14a} represents a phenyl group which is substituted at its 2-position with a halogen atom, a phenyl group which is substituted at its 4-position with a halogen atom, a 3-chlorophenyl group, a phenyl group which is substituted with two or more halogen atoms, or a pyridyl group which may be optionally substituted with one or more halogen atoms.

[Embodiment C47] The compound according to [Embodiment C41] wherein E^{a} represents R^{14a}O—, and R^{14a} represents a 2-fluorophenyl group, a 4-fluorophenyl group, a 3-chlorophenyl group, a phenyl group which is substituted with two or more fluorine atoms, or a pyridyl group which is substituted with two or more fluorine atoms.

[Embodiment C48] The compound according to [Embodiment C41] wherein E^{a} represents R^{34a}R^{35a}C═N—N═C(R^{10a})—, R^{15a}—CH(R^{16a})—O—N═C(R^{7a})—, or R^{14a}O—, R^{34a} represents a phenyl group, or a five- to six-membered aromatic heterocyclic group {the phenyl group, and the five- to six-membered aromatic heterocyclic group may be optionally substituted with one or more substituents selected from Group G}, R^{35a} represents a C1-C3 alkyl group, or a hydrogen atom, R⁷?, R^{10a}, and R^{16a} are identical to or different from each other and represent a C1-C3 alkyl group, R^{34a} and R^{35a} combine together with a carbon atom to which they are attached to form an indan-1-ylidene group, or a 3,4-dihydro-2H-1-benzofuran-4-ylidene group {the indan-1-ylidene group, and the 3,4-dihydro-2H-1-benzofuran-4-ylidene group may be optionally substituted with one or more substituents selected from Group J3}, R^{15a} represents a phenyl group which may be optionally substituted with one or more substituents selected from Group G, R^{14a} represents a phenyl group which is substituted at its 2-position with substituent(s) selected from Group L2, a phenyl group which is substituted at its 3-position with substituent(s) selected from Group M2, a phenyl group which is substituted at its 4-position with substituent(s) selected from Group L², a phenyl group which is substituted with two or more halogen atoms, or a pyridyl group which is substituted with substituent(s) selected from Group L2,
Group L2 is a group consisting of a C1-C3 alkoxy group and a halogen atom,
Group M2 is a group consisting of a C1-C3 alkyl group which is substituted with one or more halogen atoms, and a chlorine atom.

[Embodiment C49] The compound according to [Embodiment C41] wherein E^{a} represents R^{34a}R^{35a}C═N—N═C(R^{10a})—, R^{15a}—CH(R^{16a})—O—N═C(R^{7a})—, or R^{14a}O—, R^{34a} represents a phenyl group, a pyridyl group, a thiazolyl group, or a pyrazolyl group {the phenyl group, the pyridyl group, the thiazolyl group, and the pyrazolyl group may be optionally substituted with one or more substituents selected from Group G}, R^{35a} represents methyl group, or a hydrogen atom, R^{7a}, R^{10a}, and R^{16a} represent a methyl group, R^{34a} and R^{35a} may combine together with a carbon atom to which they are attached to form an indan-1-ylidene group, or a 3,4-dihydro-2H-1-benzofuran-4-ylidene group {the indan-1-ylidene group, and the 3,4-dihydro-2H-1-benzofuran-4-ylidene group may be optionally substituted with one or more substituents selected from Group J3}, R^{15a} represents a phenyl group which may be optionally substituted with one or more substituents selected from Group G, R^{14a} represents a phenyl group which is substituted at its 2-position with substituent(s) selected from Group L2, a phenyl group which is substituted at its 3-position with substituent(s) selected from Group M2, a phenyl group which is substituted at its 4-position with substituent(s) selected from Group L2, a phenyl group which is substituted with two or more halogen atoms, or a pyridyl group which may be optionally substituted with substituent(s) selected from Group L2.

[Embodiment C50] The compound according to [Embodiment C41] wherein E^{a} represents R^{14a}O—, R^{14a} represents a phenyl group which is substituted at its 2-position with substituent(s) selected from Group L3, a phenyl group which is substituted at its 3-position with substituent(s) selected from Group M3, a phenyl group which is substituted at its 4-position with substituent(s) selected from Group L³, a phenyl group which is substituted with two or more fluorine atoms, or a pyridyl group which may be optionally substituted with substituent(s) selected from Group L3,
Group L3 is a group consisting of a methoxy group, a fluorine atom, and a chlorine atom,
Group M3 is a group consisting of a trifluoromethyl group and a chlorine atom.

[Embodiment C51] The compound according to [Embodiment C41] wherein R^{14a} represents a phenyl group which may be optionally substituted with one or more substituents selected from a halogen atom and a methyl group [with the proviso that a 2-methyl phenyl group, a 3-methylphenyl group, a 4-methylphenyl group, a 3,5-dimethylphenyl group, a 3-fluorophenyl group, a 3-bromophenyl group, and a 3-iodophenyl group are excluded}.

[Embodiment C52] The compound according to [Embodiment C41] wherein E^{a} represents R^{14a}O—, and R^{14a} represents a phenyl group which is substituted with one or more chlorine atoms.

[Embodiment C53] The compound according to [Embodiment C41] wherein E^{a} represents R^{14a}O—, and R^{14a} represents a phenyl group which is substituted with one or more fluorine atoms [with the proviso that a 3-fluorophenyl group is excluded}.

[Embodiment C54] The compound according to [Embodiment C41] wherein E^{a} represents R^{14a}O—, and R^{14a} represents a phenyl group which is substituted at its 2-position with a halogen atom, a phenyl group which is substituted at its 4-position with a halogen atom, a phenyl group which is substituted with two or more halogen atoms, or a 3-chlorophenyl group.

[Embodiment C55] The compound according to [Embodiment C41] wherein E^{a} represents R^{14a}O—, and R^{14a} represents a phenyl group which is substituted at its 2-position with a halogen atom.

[Embodiment C56] The compound according to [Embodiment C41] wherein E^{a} represents R^{14a}O—, and R^{14a} represents a phenyl group which is substituted at its 4-position with a halogen atom.

[Embodiment C57] The compound according to [Embodiment C41] wherein E^{a} represents R^{14a}O—, and R^{14a} represents a phenyl group which is substituted at its 2- or 4-position with a halogen atom.

[Embodiment C58] The compound according to [Embodiment C41] wherein E^{a} represents R^{14a}O—, and R^{14a} represents a phenyl group which is substituted at its 2-position with substituent(s) selected from Group L.

[Embodiment C59] The compound according to [Embodiment C41] wherein E^{a} represents R^{14a}O—, and R^{14a} represents a phenyl group which is substituted at its 3-position with substituent(s) selected from Group M.

[Embodiment C60] The compound according to [Embodiment C41] wherein E^{a} represents R^{14a}O—, and R^{14a} represents a phenyl group which is substituted at its 4-position with substituent(s) selected from Group N.

[Embodiment C61] The compound according to [Embodiment C41] wherein E^{a} represents R^{14a}O—, and R^{14a} represents a phenyl group which is substituted at its 2- or 4-position with substituent(s) selected from Group O.

[Embodiment C62] The compound according to [Embodiment C41] wherein E^{a} represents R^{14a}O—, and R^{14a} represents a phenyl group which is substituted with two or more substituents selected from Group P.

[Embodiment C63] The compound according to [Embodiment C41] wherein E^{a} represents R^{14a}O—, and R^{14a} represents a phenyl group which is substituted at its 2-position with substituent(s) selected from Group L, a phenyl group which is substituted at its 3-position with substituent(s) selected from Group M, a phenyl group which is substituted at its 4-position with substituent(s) selected from Group N, or a phenyl group which is substituted with two or more substituents selected from Group P.

[Embodiment C64] The compound according to [Embodiment C41] wherein E^{a} represents R^{14a}O—, and R^{14a} represents a pyridyl group which may be optionally with substituent(s) selected from Group P, a pyrazolyl group which may be optionally substituted with substituent(s) selected from Group G, or a pyrimidinyl group which may be optionally substituted with substituent(s) selected from Group Q.

[Embodiment C65] The compound according to [Embodiment C41] wherein E^{a} represents R^{14a}O—, and R^{14a} represents a phenyl group which is substituted at its 2-position with substituent(s) selected from Group L, a phenyl group which is substituted at its 3-position with substituent(s) selected from Group M, a phenyl group which is substituted at its 4-position with substituent(s) selected from Group N, a phenyl group which is substituted with two or more substituents selected from Group P, a pyridyl group which may be optionally with substituent(s) selected from Group P, a pyrazolyl group which may be optionally substituted with substituent(s) selected from Group G, or a pyrimidinyl group which may be optionally substituted with substituent(s) selected from Group Q.

[Embodiment C66] The compound according to any one of [Embodiment C41] to [Embodiment C65] or the present compound C wherein Q^{a} represents a group represented by Q1.

[Embodiment C67] The compound according to any one of [Embodiment C41] to [Embodiment C65] or the present compound C wherein Q^{a} represents a group represented by Q2.

Examples of the embodiment of the present compound D include the following compounds.

[Embodiment D1] The present compound D wherein E³ represents R³⁹OCH₂—, or R⁴⁰CH₂O—.

[Embodiment D2] The present compound D wherein E³ represents R³⁹OCH₂—.

[Embodiment D3] The present compound D wherein E³ represents R⁴⁰CH₂O—.

[Embodiment D4] The present compound D1 wherein R³⁹ represents a five- to six-membered aromatic heterocyclic group which may be optionally substituted with one or more substituents selected from Group R, and R⁴⁰ represents a phenyl group which may be optionally substituted with one or more substituents selected from Group R.

[Embodiment D5] The present compound D1 wherein R³⁹ represents a pyrazolyl group, or a pyridyl group {the pyrazolyl group, and the pyridyl group may be optionally substituted with one or more substituents selected from Group W}, and R⁴⁰ represents a phenyl group which may be optionally substituted with one or more substituents selected from Group W,
Group W is a group consisting of a methyl group, a phenyl group {the methyl group, and the phenyl group may be optionally substituted with one or more halogen atoms}, and a halogen atom.

[Embodiment D6] The present compound D2 wherein R³⁹ represents a pyrazolyl group which may be optionally substituted with one or more R⁴⁴, or a pyridyl group which may be optionally substituted with one or more R⁴⁵, R⁴⁴ represents a phenyl group which may be optionally substituted with one or more halogen atoms, and R⁴⁵ represents a methyl group which may be optionally substituted with one or more halogen atoms.

[Embodiment D7] The present compound D3 wherein R⁴⁰ represents a phenyl group which may be optionally substituted with one or more substituents selected from Group Z,
Group Z is a group consisting of a methyl group which may be optionally substituted with one or more halogen atoms, and a halogen atom.

[Embodiment D14] The present compound D wherein E³ represents R⁴⁶O—.

[Embodiment D15] The present compound D wherein E³ represents R⁴¹S—.

[Embodiment D16] The present compound D wherein E³ represents R³⁹OCH₂—, R⁴⁰CH₂O—, R⁴¹S—, or R⁴⁶O—.

[Embodiment D17] The present compound D wherein E³ represents R³⁹OCH₂—, R⁴⁰CH₂O—, or R⁴⁶O—.

[Embodiment D18] The present compound D14 wherein R⁴⁶ represents a C5-C6 cycloalkyl group.

[Embodiment D19] The present compound D15 wherein R⁴¹ represents a phenyl group which may be optionally substituted with one or more halogen atoms.

[Embodiment D20] The present compound D16 wherein R³⁹ represents a pyrazolyl group which may be optionally substituted with one or more R⁴, a pyridyl group which may be optionally substituted with one or more R⁴⁵, or a phenyl group, R⁴⁴ represents a phenyl group which may be optionally substituted with one or more halogen atoms, R⁴⁵ represents a methyl group which may be optionally substituted with one or more halogen atoms, R⁴⁰ represents a phenyl group which may be optionally substituted with one or more substituents selected from Group Z, R⁴¹ represents a phenyl group which may be optionally substituted with one or more halogen atoms, and R⁴⁶ represents a C5-C6 a cycloalkyl group.

[Embodiment D21] The present compound D17 wherein R³⁹ represents a pyrazolyl group which may be optionally substituted with one or more R⁴⁴, a pyridyl group which may be optionally substituted with one or more R⁴⁵, or a phenyl group, R⁴⁴ represents a phenyl group which may be optionally substituted with one or more halogen atoms, R⁴⁵ represents methyl group which may be optionally substituted with one or more halogen atoms, R⁴⁰ represents a phenyl group which may be optionally substituted with one or more substituents selected from Group Z, and R⁴⁶ represents a C5-C6 a cycloalkyl group.

[Embodiment D8] The compound according to any one of [Embodiment D1] to [Embodiment D7], [Embodiment D14] to [Embodiment D21], or the present compound D wherein n is 0, R¹ represents a methyl group, Q represents a group represented by Q1, or a group represented by Q2, and L¹ represents an oxygen atom or NH.

[Embodiment D9] The compound according to any one of [Embodiment D1] to [Embodiment D7], [Embodiment D14] to [Embodiment D21], or the present compound D wherein n is 0, R¹ represents a methyl group, and Q represents a group represented by Q1.

[Embodiment D10] The compound according to any one of [Embodiment D1] to [Embodiment D7], [Embodiment D14] to [Embodiment D21], or the present compound D wherein n is 0, R¹ represents a methyl group, and Q represents a group represented by Q2.

[Embodiment D11] The compound according to any one of [Embodiment D1] to [Embodiment D7], [Embodiment D14] to [Embodiment D21], or the present compound D wherein n is 1, R¹ represents a hydrogen atom, and Q represents a group represented by Q1, or a group represented by Q2.

[Embodiment D12] The compound according to any one of [Embodiment D1] to [Embodiment D7], [Embodiment D14] to [Embodiment D21], or the present compound D wherein n is 1, R¹ represents a hydrogen atom, and Q represents a group represented by Q1.

[Embodiment D13] The compound according to any one of [Embodiment D1] to [Embodiment D7], [Embodiment D14] to [Embodiment D21], or the present compound D wherein n is 1, R¹ represents a hydrogen atom, and Q represents a group represented by Q2.

Next, the process for preparing the compounds disclosed herein (including the present compound C, the present compound D and the present compound A) are explained.

### Process A

A compound represented by formula (A1) (hereinafter, referred to as “Compound (A1)”) can be prepared by reacting a compound represented by formula (B1) (hereinafter, referred to as “Compound (B1)”) with a compound represented by formula (M1) (hereinafter, referred to as “Compound (M1)”) in the presence of a palladium catalyst and a base.

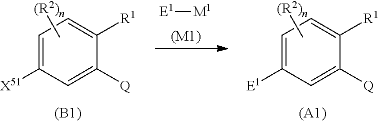

[wherein
a combination of Q and E′ represents a combination wherein Q represents a group represented by Q1, or a group represented by Q2, E′ represents a C1-C6 chain hydrocarbon group which may be optionally substituted with one or more substituents selected from Group A, a cyclopropyl group which may be optionally substituted with one or more substituents selected from Group B, or R⁴R⁵C═C(R⁶)—; or a combination wherein Q represents a group represented by Q3, a group represented by Q4, a group represented by Q5, a group represented by Q6, a group represented by Q7, or a group represented by Q8; and E¹ represents a C1-C6 chain hydrocarbon group which may be optionally substituted with one or more substituents selected from Group A, a cyclopropyl group which may be optionally substituted with one or more substituents selected from Group B, a C6-C10 aryl group which may be optionally substituted with one or more substituents selected from Group D, or a five- to ten-membered aromatic heterocyclic group which may be optionally substituted with one or more substituents selected from Group D, M1 represents B(OH)₂, or a 4,4,5,5-tetramethyl-1,3,2-dioxaborolan-2-yl group; X⁵¹ represents a leaving group such as chlorine atom, bromine atom, iodine atom, or triflyloxy group; and the other symbols are the same as defined above.]

The reaction is usually carried out in a solvent. Examples of the solvent to be used in the reaction include hydrocarbons such as hexane, toluene, and xylene (hereinafter, collectively referred to as hydrocarbons); ethers such as methyl tert-butyl ether (hereinafter, referred to as MTBE), tetrahydrofuran (hereinafter, referred to as THF), dimethoxyethane (hereinafter, collectively referred to as ethers); halogenated hydrocarbons such as chloroform and chlorobenzene (hereinafter, collectively referred to as halogenated hydrocarbons); amides such as dimethylformamide (hereinafter, referred to as DMF) and N-methyl pyrrolidone (hereinafter, collectively referred to as amides); esters such as methyl acetate and ethyl acetate (hereinafter, collectively referred to as esters); nitriles such as acetonitrile and propionitrile (hereinafter, collectively referred to as nitriles); water; and mixed solvents of these two or more solvents.

Example of the palladium catalysts includes [1,1′-bis(diphenylphoshino)ferrocene]palladium (II) dichloride.

Examples of the base include organic bases such as triethylamine and pyridine (hereinafter, collectively referred to as organic bases); alkali metal carbonates such as sodium carbonates and potassium carbonates (hereinafter, collectively referred to as alkali metal carbonates); alkali metal hydrocarbonates such as sodium hydrocarbonate and potassium hydrocarbonate (hereinafter, collectively referred to as alkali metal hydrocarbonates); sodium fluoride, and tripotassium phosphate.

In the reaction, the compound (M1) is usually used within a range of 1 to 10 molar ratio(s), the palladium catalyst is usually used within a range of 0.01 to 1 molar ratio(s), and the base is usually used within a range of 1 to 10 molar ratio(s), as opposed to 1 mole of the compound (B1).

The reaction temperature is usually within a range of 0 to 150° C. The reaction period of the reaction is usually within a range of 0.1 to 120 hours.

When the reaction is completed, water is added to reaction mixtures, and the reaction mixtures are extracted with organic solvent(s), and the organic layers are worked up (such as concentration and drying) to isolate the compound (A1).

The compound (B1) and the compound (M1) are known compounds, or can be prepared according to a known method.

### Process B

The compound (A1) can be prepared by reacting a compound represented by formula (B2) (hereinafter, referred to as “Compound (B2)”) with a compound represented by formula (M2) (hereinafter, referred to as “Compound (M2)”) in the presence of a palladium catalyst and a base.

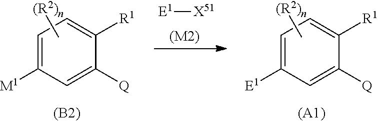

[wherein the symbols are the same as defined above.]

The reaction can be carried out by using the compound (M2) in place of the compound (M1) and using the compound (B2) in place of the compound (M2) according to the process A.

The compound (B2) and the compound (M2) are known compounds, or can be prepared according to a known method.

### Process C

A compound represented by formula (A2) (hereinafter, referred to as “Compound (A2)”) can be prepared by reacting a compound represented by formula (B36) (hereinafter, referred to as “Compound (B36)”) with a compound represented by formula (M3) (hereinafter, referred to as “Compound (M3)”) in the presence of a metal catalyst and a base.

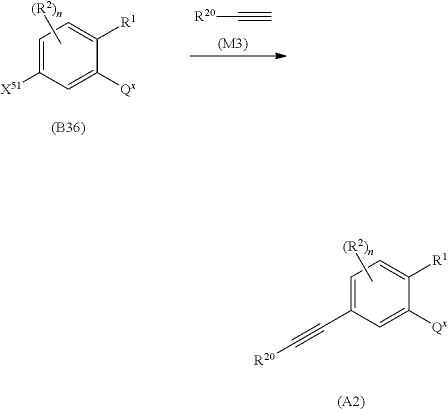

[wherein, Q^{x} represents a group represented by Q3, a group represented by Q4, a group represented by Q5, a group represented by Q6, a group represented by Q7, or a group represented by Q8, and the other symbols are the same as defined above.]

The reaction is usually carried out in a solvent. Examples of the solvents to be used in the reaction include hydrocarbons, ethers, halogenated hydrocarbons, amides, esters, nitriles, and mixed solvents of these two or more solvents.

Examples of the metal catalyst to be used in the reaction include bis(triphenylphosphine)palladium(II) dichloride (hereinafter, referred to as PdCl₂(PPh₃)₂) and a copper (I) iodide.

The base to be used in the reaction includes the organic bases.

In the reaction, the compound (M3) is usually used within a range of 1 to 10 molar ratio(s), the metal catalyst is usually used within a range of 0.01 to 1 molar ratio(s), and the base is usually used within a range of 1 to 10 molar ratio(s), as opposed to 1 mole of the compound (B36).

The reaction temperature is usually within a range of 0 to 150° C. The reaction period of the reaction is usually within a range of 0.1 to 120 hours.

When the reaction is completed, water is added to the reaction mixtures, and the organic layers are worked up (such as concentration and drying) to isolate the compound (A2).

The compound (B36) and the compound (M3) are known compounds, or can be prepared according to a known method.

### Process D

A compound represented by formula (A3) (hereinafter, referred to as “Compound (A3)”) can be prepared by reacting a compound represented by formula (B3) (hereinafter, referred to as “Compound (B3)”) with a compound represented by formula (M4) (hereinafter, referred to as “Compound (M4)”) or salts thereof.

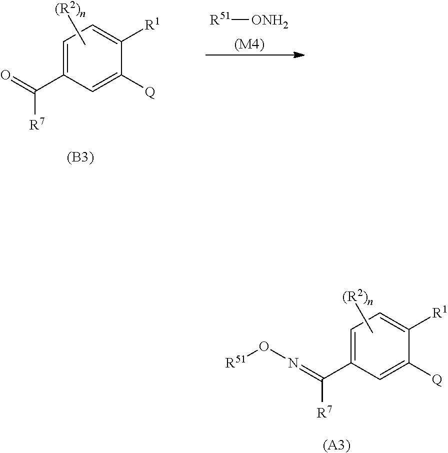

[wherein
a combination of Q and R⁵¹ represents a combination wherein Q represents a group represented by Q1, or a group represented by Q2, and R⁵¹ represents R¹⁵—CH(R¹⁶)—; or a combination wherein Q represents a group represented by Q3, a group represented by Q4, a group represented by Q5, a group represented by Q6, a group represented by Q7, or a group represented by Q8, and R⁵¹ represents R¹³; and the other symbols are the same as defined above.]

Examples of the salts of the compound (M4) include hydrochloride salts and sulfates.

The reaction is usually carried out in a solvent. Examples of the solvents to be used in the reaction include hydrocarbons, ethers, halogenated hydrocarbons, amides, esters, nitriles, alcohols (such as methanol, and ethanol), and mixed solvents of these two or more solvents.

A base may be used in the reaction as needed.

Examples of the bases to be used in the reaction include organic bases, alkali metal carbonates, alkali metal hydrocarbonates, sodium hydride and tripotassium phosphate.

When a base is used in the reaction, the base is usually within a range of 1 to 10 molar ratio(s), as opposed to 1 mole of the compound (B3).

In the reaction, the compound (M4) is usually used within a range of 1 to 10 molar ratio(s) as opposed to 1 mole of the compound (B3).

The reaction temperature is usually within a range of 0 to 150° C. The reaction period of the reaction is usually within a range of 0.1 to 120 hours.

When the reaction is completed, water is added to the reaction mixtures, and the resulting mixtures are extracted with organic solvent(s), and the organic layers are worked up (such as concentration and drying) to isolate the compound (A3).

Also the compound (A3) can be prepared according a method described in WO 1999/043949 A1, WO 2000/041999 A1, or WO 2000/007999 A1.

The compound (B3) and the compound (M4) are known compounds, or can be prepared according to a known method.

### Process E

A compound represented by formula (A4) (hereinafter, referred to as “Compound (A4)”) can be prepared by reacting the compound (B3) with a compound represented by formula (M5) (hereinafter, referred to as “Compound (M5)”) or salts thereof.

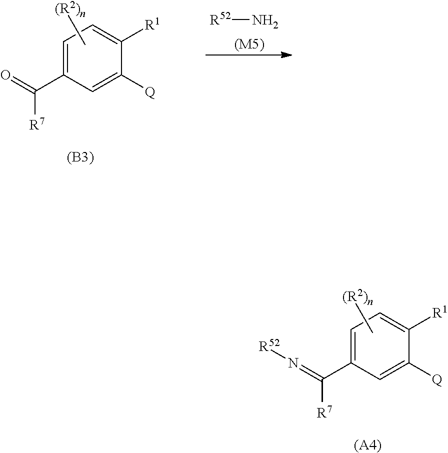

[wherein,
a combination of Q and R⁵² represents a combination wherein Q represents a group represented by Q1, or a group represented by Q2, R⁵² represents R³⁴R³⁵C═N—, or R¹²R⁹N—; or a combination wherein Q represents a group represented by Q3, a group represented by Q4, a group represented by Q5, a group represented by Q6, a group represented by Q7, or a group represented by Q8, and R⁵² represents R³⁴R³⁵C═N—, R¹²R⁹N—, or R¹⁸; and the other symbols are the same as defined above.]

Examples of the salts of the compound (M5) include hydrochloride salts and sulfates.

The reaction can be carried out by using the compound (M5) in place of the compound (M4) according to the process D.

The compound (M5) are known compounds, or can be prepared according to a known method.

### Process F

The compound (A3) can be prepared by reacting a compound represented by formula (B4) (hereinafter, referred to as “Compound (B4)”) with a compound represented by formula (M6) (hereinafter, referred to as “Compound (M6)”) in the presence of a base.

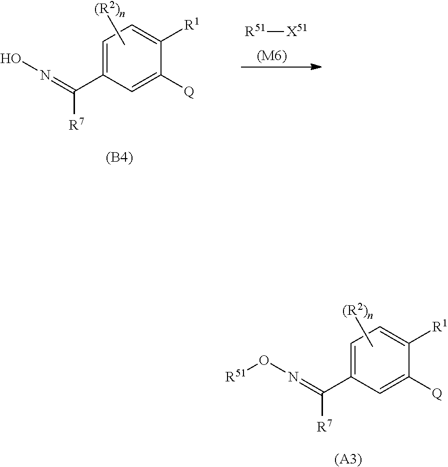

[wherein the symbols are the same as defined above.]

The reaction is usually carried out in a solvent. Examples of the solvents to be used in the reaction include hydrocarbons, ethers, halogenated hydrocarbons, amides, esters, nitriles, and mixed solvents of these two or more solvents.

Examples of the bases include organic bases, alkali metal carbonates, alkali metal hydrocarbonates, sodium hydride and tripotassium phosphate.

A metal catalyst and/or a ligand may be used in the reaction as needed.

Examples of the metal catalyst include copper catalysts (such as copper (I) iodide, copper (I) bromide, copper (I) chloride, copper (I) oxide, trifluoromethane sulfonate copper (I) benzene complex, tetrakis(acetonitrile) copper (I) hexafluorophoshate, and 2-thiophene carboxylate copper (I)); nickel catalysts (such as bis(cyclooctadine)nickel (0), and nickel (II) chloride). When the metal catalyst is used in the reaction, the metal catalyst is usually used within a range of 0.01 to 1 molar ratio(s) as opposed to 1 mole of the compound (B4).

Examples of the ligand include triphenylphosphine, Xantphos, 2,2′-bis(diphenylphosphino)-1,1′-binaphthyl, 1,1′-bis(diphenylphosphino)ferrocene, 2-dicyclohexylphosphino-2′,4′,6′-triisopropylbiphenyl, 2-dicyclohexylphosphino-2′,6′-dimethoxybiphenyl, 1,2-bis(diphenylphosphino)ethane, 2,2′-bipyridine, 2-aminoethanol, 8-hydroxyquinoline, 1,10-phenanthroline, trans-1,2-cyclohexanediamine, trans-N,N′-dimethylcyclohexane-1,2-diamine, N,N′-dimethyletylenediamine, and N,N-dimethylglycine hydrochloride salt.

When the ligand is used in the reaction, the ligand is usually used within a range of 0.1 to 1 molar ratio(s) as opposed to 1 mole of the compound (B4).

In the reaction, the compound (M6) is usually used within a range of 1 to 10 molar ratio(s), and the base is usually used within a range of 1 to 10 molar ratio(s), as opposed to 1 mole of the compound (B4).

The reaction temperature is usually within a range of −20 to 150° C. The reaction period of the reaction is usually within a range of 0.1 to 48 hours.

When the reaction is completed, water is added to the reaction mixtures, and the resulting mixtures are extracted with organic solvent(s), and the organic layers are worked up (such as concentration and drying) to isolate the compound (A3).

The compound (M6) is known compound, or can be prepared according to a known method.

### Process G

A compound represented by formula (A5) (hereinafter, referred to as “Compound (A5)”) can be reacting a compound represented by formula (B5) (hereinafter, referred to as “Compound (B5)”) with a compound represented by formula (M7) (hereinafter, referred to as “Compound (M7)”) in the presence of a base.

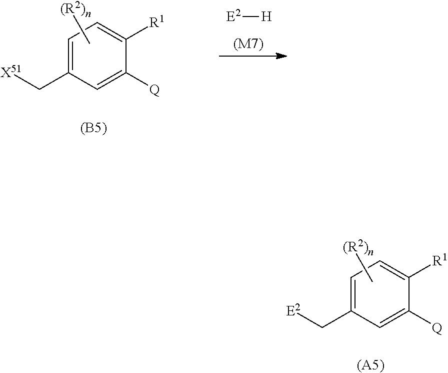

[wherein,
E² represents R⁸R⁹C═N—O—, R¹²O—N═C(R⁷)—C(R⁹)═N—O—, R¹²C(O)—C(R⁹)═N—O—, R¹²C(═N—O—R⁷)—C(R⁹)═N—O—, R¹²N═C(R⁷)—S—, R¹²N═C(SR⁷)—S—, R¹²O—N═C(R⁷)—S—, or R¹²O—N═C(SR⁷)—S—; and the other symbols are the same as defined above.]

The reaction is usually carried out in a solvent. Examples of the solvents to be used in the reaction include hydrocarbons, ethers, halogenated hydrocarbons, amides, esters, nitriles, and mixed solvents of these two or more solvents.

Examples of the bases include organic bases, alkali metal carbonates, alkali metal hydrocarbonates, sodium hydride and tripotassium phosphate.

In the reaction, the compound (M7) is usually used within a range of 1 to 10 molar ratio(s), and the base is usually used within a range of 1 to 10 molar ratio(s), as opposed to 1 mole of the compound (B5).

The reaction temperature is usually within a range of −20 to 150° C. The reaction period of the reaction is usually within a range of 0.1 to 48 hours.

When the reaction is completed, water is added to the reaction mixtures, and the resulting mixtures are extracted with organic solvent(s), and the organic layers are worked up (such as concentration and drying) to isolate the compound (A5).

Also the compound (A5) can be prepared according a method described in WO 1990/07493 A1, WO 1995/18789 A1 and so no.

The compound (M7) is a known compound, or can be prepared according to a known method.

### Process H

A compound represented by formula (AG) (hereinafter, referred to as “Compound (A6)”) can be prepared by reacting a compound represented by formula (B45) (hereinafter, referred to as “Compound (B45)”) with a compound represented by formula (M8) (hereinafter, referred to as “Compound (M8)”) in the presence of phosphines and azodiesters.

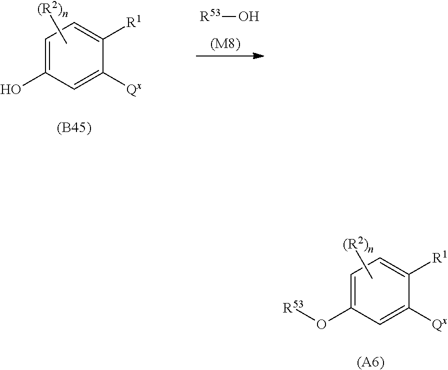

[wherein R⁵³ represents a methyl group which is substituted with one or more substituents selected from Group F, a C2-C6 chain hydrocarbon group which may be optionally substituted with one or more substituents selected from Group F, or a C3-C4 cycloalkyl group which may be optionally substituted with one or more substituents selected from Group B; and the other symbols are the same as defined above.]

The reaction is usually carried out in a solvent. Examples of the solvents to be used in the reaction include hydrocarbons, ethers, halogenated hydrocarbons, amides, esters, nitriles, and mixed solvents of these two or more solvents.

Examples of the phosphines include triphenylphosphine and trimethylphisphine.

Examples of azodiesters include diethyl azodicarboxylate, diisopropyl azodicarboxylate and bis(2-methoxyethyl)azodicarboxylate.

In the reaction, the compound (M8) is usually used within a range of 1 to 10 molar ratio(s), and the phosphines is usually used within a range of 1 to 10 molar ratio(s), and the azodiesters is usually used within a range of 1 to 10 molar ratio(s), as opposed to 1 mole of the compound (B45)

The reaction temperature is usually within a range of 0 to 150° C. The reaction period of the reaction is usually within a range of 0.1 to 48 hours.

When the reaction is completed, water is added to the reaction mixtures, and the resulting mixtures are extracted with organic solvent(s), and the organic layers are worked up (such as concentration and drying) to isolate the compound (A6).

The compound (M8) is a known compound, or can be prepared according to a known method.

### Process I

A compound represented by formula (A7) (hereinafter, referred to as “Compound (A7)”) can be prepared by reacting a compound represented by formula (B6) (hereinafter, referred to as “Compound (B6)”) with a compound represented by formula (M9) (hereinafter, referred to as “Compound (M9)”) in the presence of a base.

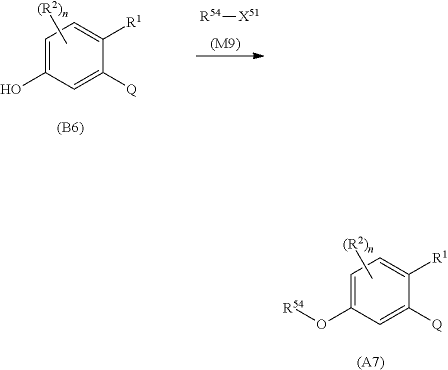

[wherein
a combination of Q and R⁵⁴ represents a combination wherein Q represents a group represented by Q1, or a group represented by Q2, R⁵⁴ represents a C6-C10 aryl group which may be optionally substituted with one or more substituents selected from Group D, or a five- to ten-membered aromatic heterocyclic group which may be optionally substituted with one or more substituents selected from Group D; or a combination wherein Q represents a group represented by Q3 to Q8, and R⁵⁴ represents a C6-C10 aryl group which may be optionally substituted with one or more substituents selected from Group D, a five- to ten-membered aromatic heterocyclic group which may be optionally substituted with one or more substituents selected from Group D, a methyl group which is substituted with one or more substituents selected from Group F, a C2-C6 chain hydrocarbon group which may be optionally substituted with one or more substituents selected from Group F, or a C3-C4 cycloalkyl group which may be optionally substituted with one or more substituents selected from Group B; and the other symbols are the same as defined above.]

The reaction is usually carried out in a solvent. Examples of the solvents to be used in the reaction include hydrocarbons, ethers, halogenated hydrocarbons, amides, esters, nitriles, and mixed solvents of these two or more solvents.

Examples of the bases include organic bases, alkali metal carbonates, alkali metal hydrocarbonates, sodium hydride, and tripotassium phosphate.

A metal catalyst and/or a ligand may be used in the reaction as needed.

Examples of the metal catalyst include copper catalysts (such as copper (I) iodide, copper (I) bromide, copper (I) chloride, copper (I) oxide, trifluoromethane sulfonate copper (I) benzene complex, tetrakis(acetonitrile) copper (I) hexafluorophoshate, and 2-thiophene carboxylate copper (I)); nickel catalysts (such as bis(cyclooctadine)nickel (0), and nickel (II) chloride). When the metal catalyst is used in the reaction, the metal catalyst is usually used within a range of 0.01 to 1 molar ratio(s) as opposed to 1 mole of the compound (B6).

Examples of the ligand include triphenylphosphine, Xantphos, 2,2′-bis(diphenylphosphino)-1,1′-binaphthyl, 1,1′-bis(diphenylphosphino)ferrocene, 2-dicyclohexylphosphino-2′,4′,6′-triisopropylbiphenyl, 2-dicyclohexylphosphino-2′,6′-dimethoxybiphenyl, 1,2-bis(diphenylphosphino)ethane, 2,2′-bipyridine, 2-aminoethanol, 8-hydroxyquinoline, 1,10-phenanthroline, trans-1,2-cyclohexanediamine, trans-N,N′-dimethylcyclohexane-1,2-diamine, N,N′-dimethyletylenediamine, and N,N-dimethylglycine hydrochloride salt.

When the ligand is used in the reaction, the ligand is usually used within a range of 0.01 to 1 molar ratio(s), as opposed to 1 mole of the compound (B6).

In the reaction, the compound (M9) is usually used within a range of 1 to 10 molar ratio(s), and the base is usually used within a range of 1 to 10 molar ratio(s), as opposed to 1 mole of the compound (B6).

The reaction temperature is usually within a range of −20 to 150° C. The reaction period of the reaction is usually within a range of 0.1 to 48 hours.

When the reaction is completed, water is added to the reaction mixtures, and the resulting mixtures are extracted with organic solvent(s), and the organic layers are worked up (such as concentration and drying) to isolate the compound (A7).

The compound (M9) is a known compound, or can be prepared according to a known method.

### Process J

The compound (A7) can be prepared by reacting the compound (B1) with a compound represented by formula (M10) (hereinafter, referred to as “Compound (M10)”) in the presence of a base.

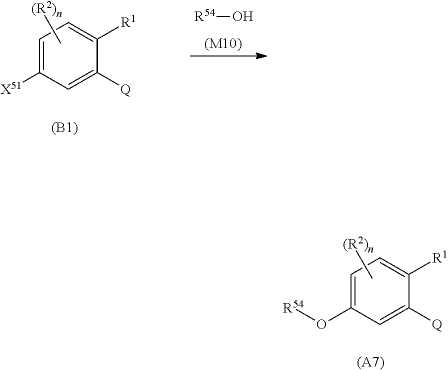

[wherein the symbols are the same as defined above.]

The reaction is usually carried out in a solvent. Examples of the solvents to be used in the reaction include hydrocarbons, ethers, halogenated hydrocarbons, amides, esters, nitriles, and mixed solvents of these two or more solvents.

Examples of the base include organic bases, alkali metal carbonates, alkali metal hydrocarbonates, sodium hydride and tripotassium phosphate.

A metal catalyst and/or a ligand may be used in the reaction as needed.

Examples of the metal catalyst include copper catalysts (such as copper (I) iodide, copper (I) bromide, copper (I) chloride, copper (I) oxide, trifluoromethane sulfonate copper (I) benzene complex, tetrakis(acetonitrile) copper (I) hexafluorophoshate, and 2-thiophene carboxylate copper (I)); nickel catalysts (such as bis(cyclooctadine)nickel (0), and nickel (II) chloride). When the metal catalyst is used in the reaction, the metal catalyst is usually used within a range of 0.01 to 1 molar ratio(s) as opposed to 1 mole of the compound (B1).

Examples of the ligand include triphenylphosphine, Xantphos, 2,2′-bis(diphenylphosphino)-1,1′-binaphthyl, 1,1′-bis(diphenylphosphino)ferrocene, 2-dicyclohexylphosphino-2′,4′,6′-triisopropylbiphenyl, 2-dicyclohexylphosphino-2′,6′-dimethoxybiphenyl, 1,2-bis(diphenylphosphino)ethane, 2,2′-bipyridine, 2-aminoethanol, 8-hydroxyquinoline, 1,10-phenanthroline, trans-1,2-cyclohexanediamine, trans-N,N′-dimethylcyclohexane-1,2-diamine, N,N′-dimethyletylenediamine, and N,N-dimethylglycine hydrochloride salt.

When the ligand is used in the reaction, the ligand is usually used within a range of 0.01 to 1 molar ratio(s), as opposed to 1 mole of the compound (B1).

In the reaction, the compound (M10) is usually used within a range of 1 to 10 molar ratio(s), and the base is usually used within a range of 1 to 10 molar ratio(s), as opposed to 1 mole of the compound (B1).

The reaction temperature is usually within a range of −20 to 150° C. The reaction period of the reaction is usually within a range of 0.1 to 48 hours.

When the reaction is completed, water is added to the reaction mixtures, and the resulting mixtures are extracted with organic solvent(s), and the organic layers are worked up (such as concentration and drying) to isolate the compound (A7).

The compound (M10) is a known compound, or can be prepared according to a known method.

### Process K

A compound represented by formula (A8) (hereinafter, referred to as “Compound (A8)”) can be prepared by reacting the compound (B6) with a compound represented by formula (M11) (hereinafter, referred to as “Compound (M11)”) in the presence of a base.

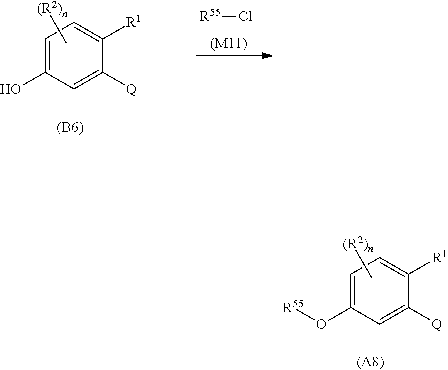

[wherein a combination of Q and R⁵⁵ represents a combination wherein Q represents a group represented by Q1, or a group represented by Q2, and R⁵⁵ represents R¹⁷R⁷NC(S)—, R¹⁷O—C(O)—, or R¹⁷C(O)—; or a combination wherein Q represents a group represented by Q3, a group represented by Q4, a group represented by Q5, a group represented by Q6, a group represented by Q7, or a group represented by Q8, R⁵⁵ represents R¹⁷R⁷NC(S)—, R¹⁷O—C(O)—, R¹⁷C(O)—, or R¹⁷R⁷NC(O)—; and the other symbols are the same as defined above.]

The reaction is usually carried out in a solvent. Examples of the solvents to be used in the reaction include hydrocarbons, ethers, halogenated hydrocarbons, amides, esters, nitriles, and mixed solvents of these two or more solvents.

Examples of the bases include organic bases, alkali metal carbonates, alkali metal hydrocarbonates, sodium hydride, and tripotassium phosphate.

In the reaction, the compound (M11) is usually used within a range of 1 to 10 molar ratio(s), and the base is usually used within a range of 1 to 10 molar ratio(s), as opposed to 1 mole of the compound (B6).

The reaction temperature is usually within a range of −78 to 100° C. The reaction period of the reaction is usually within a range of 0.1 to 48 hours.

When the reaction is completed, water is added to the reaction mixtures, and the resulting mixtures are extracted with organic solvent(s), and the organic layers are worked up (such as concentration and drying) to isolate the compound (A8).

The compound (M11) is a known compound, or can be prepared according to a known method.

### Process L

A compound represented by formula (A9) (hereinafter, referred to as “Compound (A9)”) can be prepared by a step where a compound represented by formula (B7) (hereinafter, referred to as “Compound (B7)”) and a compound represented by formula (M12) (hereinafter, referred to as “Compound (M12)”) are reacted in the presence of a base to obtain a compound represented by (B8) (hereinafter, referred to as “Compound (B8)”) (hereinafter, referred to as Step (L-1)) and a step where the compound (B8) and a compound represented by formula (M13) (hereinafter, referred to as “Compound (M13)”) in the presence of a base (hereinafter, referred to as Step (L-2))

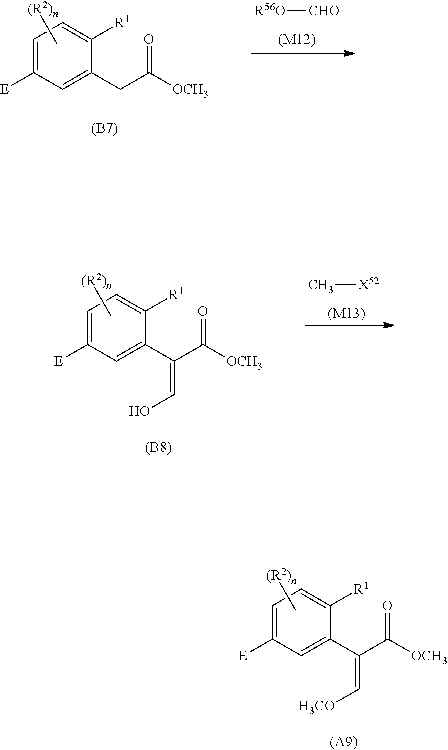

[wherein R⁵⁶ represents a C1-C4 alkyl group, X⁵² represents an iodine atom, a methoxysulfonyloxy group, a mesyloxy group, or a tosyl group; and the other symbols are the same defined above.]

A step (L-1) is usually carried out is a solvent. Examples of the solvent to be used in the reaction include ethers, amides, and mixed solvents of these two or more solvents.

Examples of the base to be used in the reaction include sodium hydride and alkali metal hydrides.

In the reaction, the compound (M12) is usually used within a range of 1 to 10 molar ratio(s), and the base is usually used within a range of 0.5 to 5 molar ratio(s), as opposed to 1 mole of the compound (B7).

The reaction period of the reaction is usually within a range of 5 minutes to 72 hours. The reaction temperature is usually within a range of −20 to 100° C.

When the reaction is completed, water is added to the reaction mixtures, and the resulting mixtures are extracted with organic solvent(s), and the organic layers are worked up (such as concentration and drying) to isolate the compound (B8).

The compound (B7) and the compound (M12) are commercially available compounds, or can be prepared according to a known method.

A step (L-2) is usually carried out is a solvent. Examples of the solvent to be used in the reaction include hydrocarbons, ethers, halogenated hydrocarbons, amides, esters, nitriles and mixed solvents of these two or more solvents.

Examples of the base to be used in the reaction include organic bases, alkali metal carbonates, alkali metal hydrocarbonates, sodium hydride, and mixed solvents of these two or more solvents.

In the reaction, the compound (M13) is usually used within a range of 1 to 10 molar ratio(s), and the base is usually used within a range of 1 to 20 molar ratio(s), as opposed to 1 mole of the compound (B8).

The reaction temperature is usually within a range of −20 to 100° C. The reaction period of the reaction is usually within a range of 0.1 to 48 hours.

When the reaction is completed, water is added to the reaction mixtures, and the resulting mixtures are extracted with organic solvent(s), and the organic layers are worked up (such as concentration and drying) to isolate the compound (A9).

### Process M

A compound represented by formula (A10) (hereinafter, referred to as “Compound (A10)”) can be prepared by a step where a compound represented by formula (B43) (hereinafter, referred to as “Compound (B43)”) and a compound represented by formula (M14) (hereinafter, referred to as “Compound (M14)”) are reacted in the presence of a base to obtain a compound represented by formula (B9) (hereinafter, referred to as “Compound (B9)”) (hereinafter, referred to Step (M-1)) and a step where a compound (B9) and a compound (M13) are reacted in the presence of a base (hereinafter, referred to as Step (M-2)) in the presence of a base.

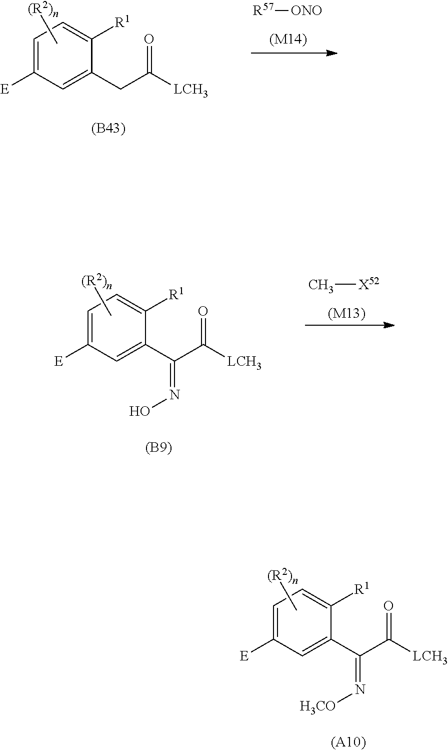

[wherein L represents an oxygen atom or NH, R⁵⁷ represents a t-butyl group, or an isopentyl group; and the other symbols are the same as defined above.]

A step (M-1) is usually carried out is a solvent. Examples of the solvent to be used in the reaction include ethers, amides, alcohols, and mixed solvents of these two or more solvents.

Examples of the base to be used in the reaction include sodium hydride and alkali metal alkoxides (such as sodium methoxide, sodium ethoxide, and potassium t-butoxide)

In the reaction, the compound (M14) is usually used within a range of 1 to 10 molar ratio(s), and the base is usually used within a range of 1 to 5 molar ratio(s), as opposed to 1 mole of the compound (B43).

The reaction period of the reaction is usually within a range of 5 minutes to 72 hours. The reaction temperature is usually within a range of −20 to 100° C.

When the reaction is completed, water is added to the reaction mixtures, and the resulting mixtures are extracted with organic solvent(s), and the organic layers are worked up (such as concentration and drying) to isolate the compound (B9).

The compound (B43) and the compound (M14) are commercially available compounds, or can be prepared according to a known method.

A step (M-2) can be carried out by using the compound (B9) in place of the compound (B8) according to the step (L-2) of the Process L.

The compound (M13) are known compounds, or can be prepared according to a known method.

### Process N

A compound represented by formula (A12) (hereinafter, referred to as “Compound (A12)”) can be prepared by reacting a compound represented by formula (A11) (hereinafter, referred to as “Compound (A11)”) with methyl amine.

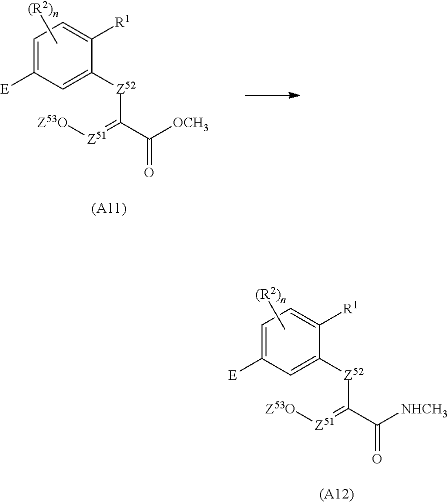

[wherein a combination of Z⁵¹, Z⁵² and Z⁵³ represents a combination where Z⁵¹ represents a nitrogen atom, Z⁵² represents an oxygen atom, and Z⁵³ represents a methyl group, a combination where Z⁵¹ represents a nitrogen atom or CH, Z⁵² represents Z¹, and Z⁵³ represents Z², or a combination where Z⁵¹ represents a nitrogen atom, Z⁵² represents a single bond, and Z⁵³ represents a methyl group; and the other symbols are the same as defined above.]

The reaction is usually carried out in a solvent. Examples of the solvents to be used in the reaction include alcohols, hydrocarbons, ethers, halogenated hydrocarbons, amides, esters, nitriles and mixed solvents of these two or more solvents.

A base may be used in the reaction as needed. Examples of the base to be used in the reaction include organic bases; alkali metal carbonates; alkali metal hydroxides (such as sodium hydroxide, and potassium hydroxide) (hereinafter, referred to as “alkali metal hydroxides”) and sodium hydride. When a base is used in the reaction, the base is usually used within a range of 0.1 to 10 molar ratio(s), as opposed to 1 mole of the compound (A11).

In the reaction, methylamine is usually used within a range of 1 to 100 molar ratio(s) as opposed to 1 mole of the compound (A11).

The reaction temperature is usually within a range of −20 to 150° C. The reaction period of the reaction is usually within a range of 0.1 to 120 hours.

When the reaction is completed, water is added to the reaction mixtures, and the resulting mixtures are extracted with organic solvent(s), and the organic layers are worked up (such as concentration and drying) to isolate the compound (A12).

### Process O

A compound represented by formula (A14) (hereinafter, referred to as “Compound (A14)”) can be prepared by a step where a compound represented by formula (A13) (hereinafter, referred to as “Compound (A13)”) and hydroxyl amine are reacted in the presence of a base to obtain a compound represented by formula (B26) (hereinafter, referred to as “Compound (B26)”) (hereinafter, referred to as Step (O-1)”) and a step where the compound (B26) and a compound represented by formula (M18) (hereinafter, referred to as “Compound (M18)”) are reacted in the presence of a base (hereinafter, referred to as Step (O-2)”).

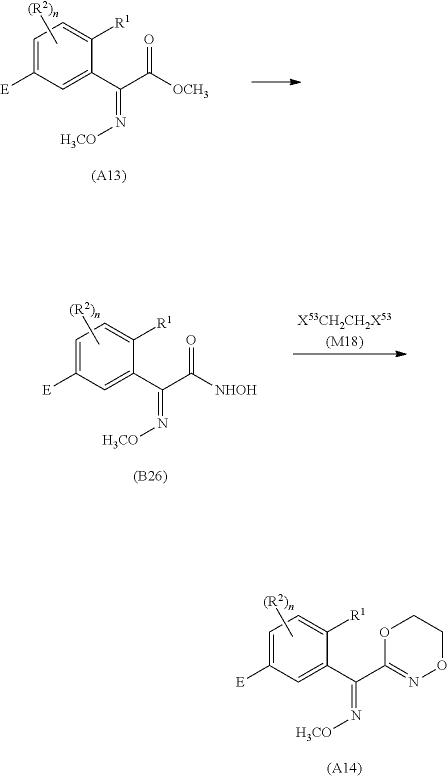

[wherein X⁵³ represents a chlorine atom, bromine atom, or iodine atom, and the other symbols are the same as defined above.]

The step (O-1) can be carried out by using hydroxylamine in place of methylamine according to the process N.

The step (O-2) is usually carried out in a solvent. Examples of the solvents to be used in the reaction include hydrocarbons, ethers, halogenated hydrocarbons, amides, esters, nitriles, water and mixed solvents of these two or more solvents.

Examples of the base include organic bases, alkali metal carbonates, alkali metal hydrocarbonates, sodium hydride and tripotassium phosphate.

In the reaction, the compound (M18) is usually used within a range of 1 to 10 molar ratio(s), and the base is usually used within a range of 1 to 10 molar ratio(s), as opposed to 1 mole of the compound (B26).

The reaction temperature is usually within a range of −20 to 150° C. The reaction period of the reaction is usually within a range of 0.1 to 48 hours.

When the reaction is completed, water is added to the reaction mixtures, and the resulting mixtures are extracted with organic solvent(s), and the organic layers are worked up (such as concentration and drying) to isolate the compound (A14).

The compound (M18) is a known compound, or can be prepared according to a known method.

### Process P

A compound represented by formula (A15) (hereinafter, referred to as “Compound (A15)”) can be prepared by a step where a compound represented by formula (B27) (hereinafter, referred to as “Compound (B27)”) and an isocyanate agent are reacted to obtain a compound represented by formula (B28) (hereinafter, referred to as “Compound (B28)”) (hereinafter, referred to as Step (P-1)), a step wherein the compound (B28) and an azide agent are reacted to obtain a compound represented by formula (B29) (hereinafter, referred to as “Compound (B29)”) (hereinafter, referred to as Step (P-2)), and a step where the compound (B29) and the compound (M13) are reacted in the presence of a base (hereinafter, referred to as “Compound (P-3)”).

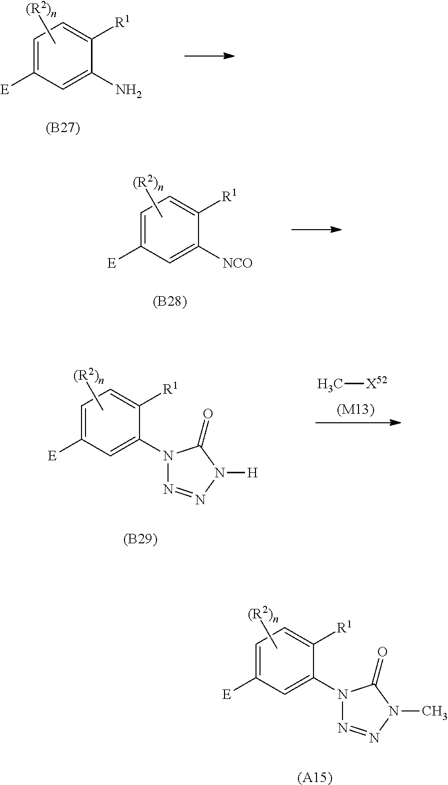

[wherein the symbols are the same as defined above]

The step (P-1) is usually carried out in a solvent. Examples of the solvents to be used in the reaction include alcohols, hydrocarbons, ethers, halogenated hydrocarbons, amides, esters, nitriles, and mixed solvents of these two or more solvents.

Examples of the isocyanate agent include phosgene, diphosgene, triphosgene, thiophosgene, N,N-carbodiimidazole, and N,N-thiocarbonyldiimidazole. These isocyanate agent is usually used within a range of 1 to 10 molar ratio(s) as opposed to 1 mole of Compound (B27).

A base may be used in the reaction as needed. Examples of the base to be used in the reaction include organic bases, alkali metal carbonates, alkali metal hydroxides, and sodium hydride. These bases are usualy used within a range of 0.05 to 5 molar ratio(s) as opposed to 1 mole of the compound (B27).

The reaction temperature is usually within a range of −20 to 150° C. The reaction period of the reaction is usually within a range of 0.1 to 48 hours.

When the reaction is completed, water is added to the reaction mixtures, and the resulting mixtures are extracted with organic solvent(s), and the organic layers are worked up (such as concentration and drying) to isolate the compound (B28).

The compound (B27) is a commercially available compound, or can be prepared according to a known method.

The step (P-2) is usually carried out in a solvent. Examples of the solvent include alcohols, hydrocarbons, ethers, halogenated hydrocarbons, amides, esters, nitriles and mixed solvents of these two or more solvents.

Examples of the azide agent to be used in the reaction include inorganic azides (such as sodium azide, barium azide, and lithium azide); and organic azides (such as trimethylsilyl azide, and diphenylphosphoryl azide). The azide agent is usually used within a range of 1 to 10 molar ratio(s) as opposed to 1 mole of the compound (B28).

A Lewis acid may be used in the reaction as needed. Examples of the Lewis acid to be used in the reaction include aluminium (III) chloride and zinc chloride. The Lewis acid is usually used within a range of 0.05 to 5 molar ratio(s), as opposed to 1 mole of the compound (B28).

The reaction temperature is usually within a range of −20 to 150° C. The reaction period of the reaction is usually within a range of 0.1 to 48 hours.

When the reaction is completed, water is added to the reaction mixtures, and the resulting mixtures are extracted with organic solvent(s), and the organic layers are worked up (such as concentration and drying) to isolate the compound (B29).

The step (P-3) is usually carried out in a solvent. Examples of the solvent include alcohols, hydrocarbons, ethers, halogenated hydrocarbons, amides, esters, nitriles and mixed solvents of these two or more solvents.

Examples of the base to be used in the reaction include organic bases, alkali metal carbonates, alkali metal hydrocarbonates, sodium hydride, and mixed solvents of these two or more solvents.

In the reaction, the compound (M13) are usually used within a range of 1 to 10 molar ratio(s), and the base is usually used within a range of 1 to 10 molar ratio(s), as opposed to 1 mole of the compound (B29).

The reaction temperature is usually within a range of −20 to 150° C. The reaction period of the reaction is usually within a range of 0.1 to 48 hours.

When the reaction is completed, water is added to the reaction mixtures, and the resulting mixtures are extracted with organic solvent(s), and the organic layers are worked up (such as concentration and drying) to isolate the compound (A15).

### Process Q

A compound represented by formula (A16) (hereinafter, referred to as “Compound (A16)”) can be prepared by a step where a compound represented by formula (B44) (hereinafter, referred to as “Compound (B44)”) and hydrazine are reacted to obtain a compound represented by formula (B30) (hereinafter, referred to as “Compound (B30)”) (hereinafter, referred to as “Step (Q-1)”), a step where the compound (B30) and a compound represented by formula (M16) (hereinafter, referred to as “Compound (M16)”) are reacted to obtain a compound represented by formula (B31) (hereinafter, referred to as “Compound (B31)”) (hereinafter, referred to as “Step (Q-2)”), and a step where the compound (B31) and a phosgene are reacted in the presence of a base (hereinafter, referred to as “Step (Q-3)”).

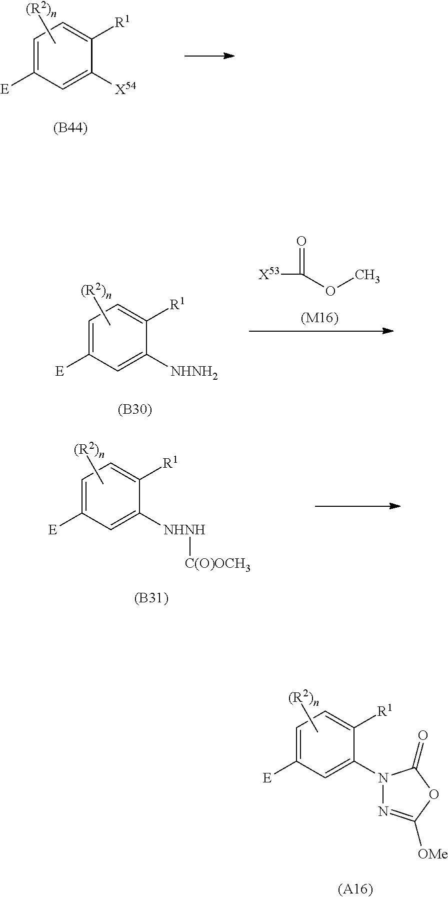

[wherein
X⁵⁴ represents a chlorine atom, bromine atom, or iodine atom; and the other symbols are the same as defined above.]

The step (Q-1) is usually carried out in a solvent. Examples of the solvent include alcohols, hydrocarbons, ethers, halogenated hydrocarbons, amides, esters, nitriles, water, and mixed solvents of these two or more solvents.

The hydrazine to be used in the reaction can be in the form of hydrazine monohydrate, hydrazine hydrochloride salt, hydrazine sulfate, anhydrous hydrazine.

In the reaction, hydrazine is used within a range of 1 to 100 molar ratio(s) as opposed to 1 mole of the compound (B29)

The reaction temperature is usually within a range of −20 to 150° C. The reaction period of the reaction is usually within a range of 0.1 to 48 hours.

When the reaction is completed, water is added to the reaction mixtures, and the resulting mixtures are extracted with organic solvent(s), and the organic layers are worked up (such as concentration and drying) to isolate the compound (B30).

The compound (B44) is a commercially available compound, or can be prepared according to a known method.

The step (Q-2) is usually carried out in a solvent. Examples of the solvent include alcohols, hydrocarbons, ethers, halogenated hydrocarbons, amides, esters, nitriles, and mixed solvents of these two or more solvents.

In the reaction, the compound (M14) is used within a range of 1 to 10 molar ratio(s) as opposed to 1 mole of the compound (B30).

A base may be used in the reaction as needed. Examples of the base to be used in the reaction include organic bases, alkali metal carbonates, alkali metal hydroxides, and sodium hydride. When a base is used in the reaction, the base is usually used within a range of 0.05 to 5 molar ratio(s) as opposed to 1 mole of the compound (B30).

The reaction temperature is usually within a range of −20 to 150° C. The reaction period of the reaction is usually within a range of 0.1 to 48 hours.

When the reaction is completed, water is added to the reaction mixtures, and the resulting mixtures are extracted with organic solvent(s), and the organic layers are worked up (such as concentration and drying) to isolate the compound (B31).

The compound (M16) is a known compound, or can be prepared according to a known method.

The step (Q-3) can be carried out by using the compound (B31) in place of the compound (B28) according to the step (P-2) of the process P.

Also the compound (A16) can be prepared according to the method described in JP 2006-151865 A1 or JP 2006-160620 A1.

### Process R

The present compound wherein Q represents a group represented by Q7, or a group represented by Q8 can be prepared according to a method described in WO 99/67209 A1, JP 2000-103773 A1, or JP H11-286472 A1.

### Process S

A N-oxide of the compound represented by formula (I) can be prepared by reacting a compound represented by formula (I) and an oxidizing agent. The reaction can be carried out by a method described in US patent publication No. 2018/0009778 A1 or WO 2016/121970 A1.

### Process T

A compound represented by formula (A17) (hereinafter, referred to as “Compound (A17)”) can be prepared by reacting a compound represented by formula (A1-T) (hereinafter, referred to as “Compound (A1-T)”) with a reducing agent in the presence of a catalyst.

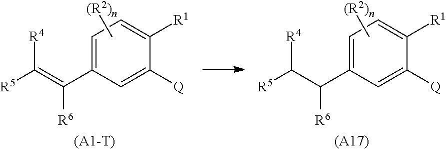

[wherein the symbols are the same as defined above.]

The reaction is usually carried out in a solvent. Examples of the solvent to be used in the reaction include hydrocarbons, ethers, halogenated hydrocarbons, amides, esters, nitriles, alcohols, and mixed solvents of these two or more solvents.

Examples of the catalyst to be used in the reaction include palladium/carbon, and palladium hydroxide.

Examples of the reducing agent to be used in the reaction include hydrogen, ammonium formate, and hydrazine.

In the reaction, the reducing agent is usually used within a range of 1 to 1000 molar ratio(s), and the catalyst is usually used within a range of 0.01 to 10 molar ratio(s), as opposed to 1 mole of the compound (A1-T).

The reaction temperature is usually within a range of −78 to 100° C. The reaction period of the reaction is usually within a range of 0.1 to 48 hours.

When the reaction is completed, water is added to the reaction mixtures, and the resulting mixtures are extracted with organic solvent(s), and the organic layers are worked up (such as concentration and drying) to isolate the compound (A17).

### Process U

A compound represented by formula (A18) (hereinafter, referred to as “Compound (A18)”) can be prepared by reacting a compound represented by formula (B50) (hereinafter, referred to as “Compound (B50)”) with a compound represented by formula (M19) (hereinafter, referred to as “Compound (M19)”) in the presence of a catalyst and a base.

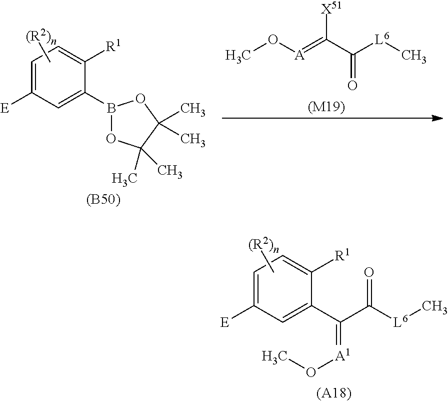

[wherein a combination of A1 and L⁶ represents a combination where A1 represents CH, and L⁶ represents an oxygen atom; a combination where A1 represents a nitrogen atom, and L⁶ represents an oxygen atom or NH; and the other symbols are the same as defined above.]

The reaction is usually carried out in a solvent. Examples of the solvent to be used in the reaction include hydrocarbons, ethers, halogenated hydrocarbons, amides, esters, nitriles, water, and mixed solvents of these two or more solvents.

Examples of the catalyst to be used in the reaction include a palladium catalysts (such as tris(dibenzylideneacetone)dipalladium (0), tetrakis(triphenylphosphine)palladium (0), and [1,1′-bis(diphenylphosphino)ferrocene] palladium (II) dichloride)

Examples of the base to be used in the reaction include organic bases; alkali metal carbonates; alkali metal hydrocarbonates; alkali metal phosphates (such as tripotassium phosphate); and acetates (such as sodium acetate).

A ligand may be used in the reaction as needed. Examples of the ligand to be used in the reaction include triphenylphosphine, Xantphos, 2,2′-bis(diphenylphosphino)-1,1′-binaphthyl, 1,1′-bis(diphenylphosphino)ferrocene, 2-dicyclohexylphosphino-2′,4′,6′-triisopropylbiphenyl, 2-dicyclohexylphosphino-2′,6′-dimethoxybiphenyl, and 1,2-bis(diphenylphosphino)ethane.

When a ligand is used in the reaction, the ligand is usually used within a range of 0.01 to 1 molar ratio(s) as opposed to 1 mole of the compound (B50).

In the reaction, the compound (M19) is usually used within a range of 0.1 to 10 molar ratio(s), the catalyst is usually used within a range of 0.01 to 1 molar ratio(s), and the base is usually used within a range of 1 to 10 molar ration(s), as opposed to 1 mole of the compound (B50).

The reaction period of the reaction is usually within a range of 5 minutes to 72 hours. The reaction temperature is usually within a range of −20 to 150° C.

When the reaction is completed, water is added to the reaction mixtures, and the resulting mixtures are extracted with organic solvent(s), and the organic layers are worked up (such as concentration and drying) to isolate the compound (A18).

The compound (M19) is a known compound, or can be prepared according to a known method.

### Reference Process 1

A compound represented by formula (B12) (hereinafter, referred to as “Compound (B12)”) can be prepared by a step where a compound represented by formula (B10) (hereinafter, referred to as “Compound (B10)”) and a compound represented by formula (M12) (hereinafter, referred to as “Compound (M12)”) are reacted in the presence of a base to obtain a compound represented by formula (B11) (hereinafter, referred to as “Compound (B11)”) (hereinafter, referred to as “Step (1-1)”), and a step where the compound (B11) and the compound (M13) are reacted in the presence of a base (hereinafter, referred to as “Step (1-2)”).

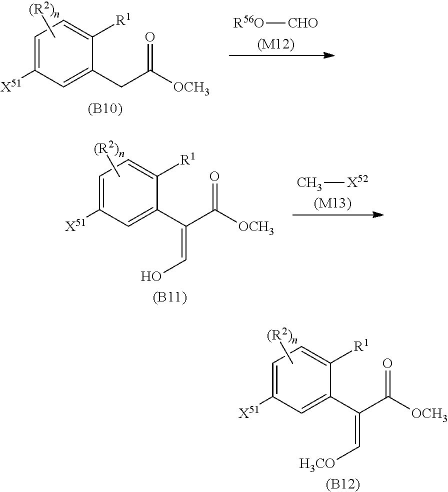

[wherein the symbols are the same as defined above.]

The step (1-1) can be carried out by using the compound (B10) in place of the compound (B7) according to the step (L-1) of the Process L.

The step (1-2) can be carried out by using the compound (B11) in place of the compound (B8) according to the step (L-2) of the Process L.

### Reference Process 2

A compound represented by formula (B15) (hereinafter, referred to as “Compound (B15)”) can be prepared by a step where a compound represented by formula (B13) (hereinafter, referred to as “Compound (B13)”) and a compound represented by formula (M14) (hereinafter, referred to as “Compound (M14)”) are reacted in the presence of a base to obtain a compound represented by formula (B14) (hereinafter, referred to as “Compound (B14)”) (hereinafter, referred to as “Step (2-1)”), and a step where the compound (B14) and the compound (M13) are reacted in the presence of a base (hereinafter, referred to as “Step (2-2)”).

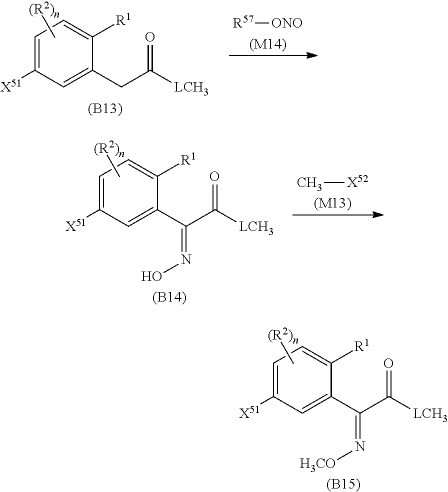

[wherein the symbols are the same as defined above.]

The step (2-1) can be carried out by using the compound (B14) in place of the compound (B43) according to the step (M-1) of the process M.

The step (2-2) can be carried out by using the compound (B14) in place of the compound (B9) according to the step (M-2) of the process M.

### Reference Process 3

A compound represented by formula (B16) (hereinafter, referred to as “Compound (B16)”) can be prepared reacting the compound (B1) with bis(pinacolato)diboron in the presence of a base and a palladium catalyst.

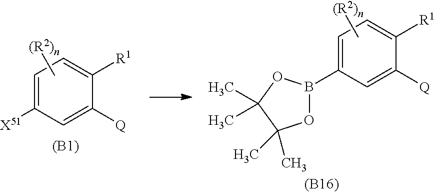

[wherein the symbols are the same defined above.]

The reaction is usually carried out in a solvent. Examples of the solvent to be used in the reaction include hydrocarbons, ethers, halogenated hydrocarbons, amides, esters, sulfoxides (such as dimethylsulfoxide (hereinafter, referred to as DMSO)) (hereinafter, referred to as sulfoxides), nitriles and mixed solvents of these two or more solvents.

Examples of the base to be used in the reaction include organic bases, alkali metal carbonates, alkali metal hydrocarbonates and tripotassium phosphate.

Examples of the palladium catalyst include [1,1′-bis(diphenylphosphino)ferrocene]palladium (II) dichloride.

In the reaction, bis(pinacolato)diboron is usually used within a range of 1 to 5 molar ratio(s), the base is usually used within a range of 1 to 5 molar ratio(s), and the palladium catalyst is usually used within a range of 0.01 to 0.5 molar ratios, as opposed to 1 mole of the compound (B1).

The reaction temperature is usually within a range of 0 to 150° C. The reaction period of the reaction is usually within a range of 0.1 to 48 hours.

When the reaction is completed, water is added to the reaction mixtures, and the resulting mixtures are extracted with organic solvent(s), and the organic layers are worked up (such as concentration and drying) to isolate the compound (B16).

### Reference Process 4

The compound (B6) can be prepared by oxidizing the compound (B16).

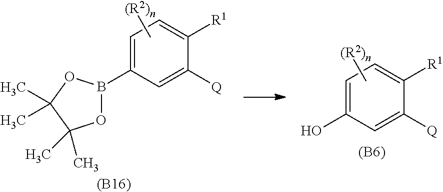

[wherein the symbols are the same as defined above.]

The reaction is usually carried out in a solvent. Examples of the solvent to be used in the reaction include hydrocarbons, ethers, halogenated hydrocarbons, amides, esters, nitriles, alcohols, water, and mixed solvents of these two or more solvents.

Examples of the oxidizing agent to be used in the reaction include meta chloroperbenzoic acid, and aqueous hydrogen peroxide solution.

When the aqueous hydrogen peroxide solution is used as an oxidizing agent, a base may be added as needed.

Examples of the base include alkali metal hydroxides.

When a base is used in the reaction, the base is usually used within a range of 0.1 to 5 molar ratios as opposed to 1 mole of the compound (B16).

In the reaction, the oxidizing agent is usually used within a range of 1 to 5 molar ratio(s) as opposed to 1 mole of the compound (B16).

The reaction temperature is usually within a range of −20 to 120° C. The reaction period of the reaction is usually within a range of 0.1 to 48 hours.

When the reaction is completed, water and a reducing agent (such as sodium thiosulfate) are added to the reaction mixture, and the resulting mixture was extracted with organic solvent(s), and the organic layers are worked up (for example, drying and concentration) to isolate the compound (B6).

### Reference Process 5

A compound represented by formula (B17) (hereinafter, referred to as “Compound (B17)”) can be prepared by reacting the compound (B1) with a compound represented by formula (M15) (hereinafter, referred to as “Compound (M15)”).

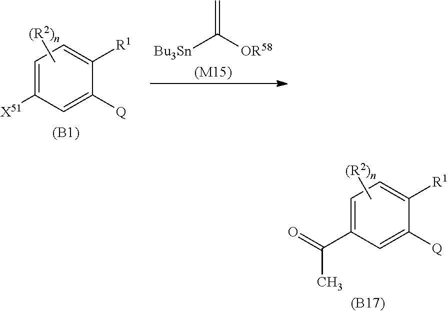

[wherein R⁵¹ represents a methyl group, or ethyl group, and the other symbols are the same as defined above.]

The reaction can be carried out according to the method described in, for example, WO 2016/123253 A1.

### Reference Process 6

The compound (B4) can be prepared by reacting the compound (B3) with hydroxylamine or salts thereof.

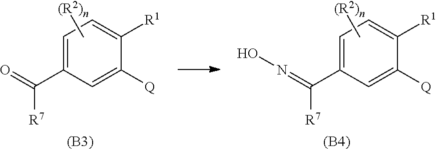

[wherein the symbols are the same as defined above.]

Examples of the salts of hydroxylamine include hydrochloride salts and sulfates.

The reaction can be carried out by using hydroxylamine in place of the compound (M4) according to the process D.

### Reference Process 7

A compound represented by formula (B19) (hereinafter, referred to as “Compound (B19)”) can be prepared by reacting a compound represented by formula (B18) (hereinafter, referred to as “Compound (B18)”) with carbon tetrachloride, carbon tetrabromide, or iodine in the presence of triphenylphosphine.

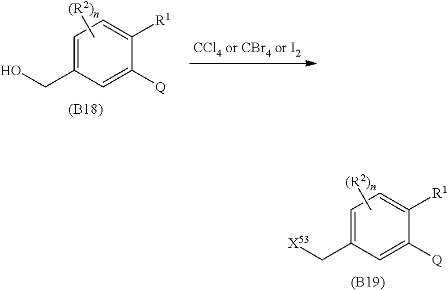

[wherein the symbols are the same as defined above.]

The reaction can be carried out according to the method described in J. Org. Synth., 1974, 54, 63.

### Reference Process 8

The compound (B18) can be prepared by a step where the compound (B1) and N-formylsaccharine in the presence of a palladium catalyst, a ligand, triethylsilane, and a base to obtain a compound represented by formula (B20) (hereinafter, referred to as “Compound (B20)”) (hereinafter, referred to as “Step (8-1)”) and a step where the compound (B20) and sodium borohydride are reacted (hereinafter, referred to as “Step (8-2)”).

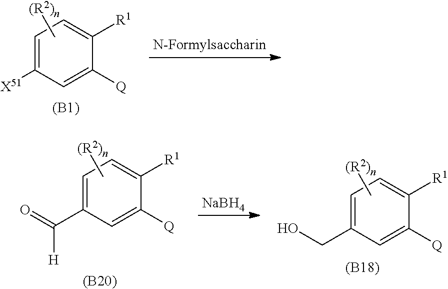

[wherein the symbols are the same as defined above.]

The step (8-1) can be carried out according to a method described in Angew. Chem. Int. Ed., 2013, 52, 8611-8615.

The step (8-2) can be carried out according to a method described in Chemistry-A European Journal, 2019, 25 (15), 3950-3956.

### Reference Process 9

A compound represented by formula (B23) (hereinafter, referred to as “Compound (B23)”) can be prepared by as step where a compound represented by formula (B21) (hereinafter, referred to as “Compound (B21)”) and triphosgene are reacted to obtain a compound represented by formula (B22) (hereinafter, referred to as “Compound (B22)”) (hereinafter, referred to as “Step (9-1)”) and a step where the compound (B22) and N,N-dimethyl hydrazine are reacted (hereinafter, referred to as “Step (9-2)”).

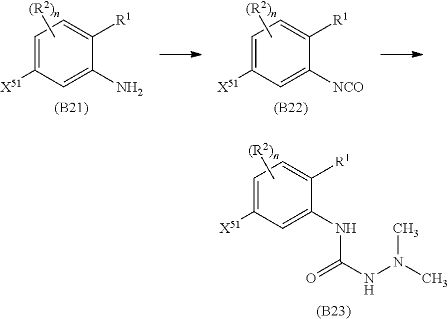

[wherein the symbols are the same as defined above.]

The step (9-1) is usually carried out in a solvent. Examples of the solvents to be used in the reaction include hydrocarbons, halogenated hydrocarbons, ethers and mixed solvents of these two or more solvents.

In the reaction, triphosgene is usually used within a range of 0.3 to 5 molar ratios as opposed to 1 mole of the compound (B21).

The reaction period of the reaction is usually within a range of 5 minutes to 72 hours. The reaction temperature is usually within a range of 0 to 150° C.

When the reaction is completed, the reaction mixture is worked up (for example, concentration) to obtain the compound (B22).

The compound (B21) is a commercially available compound, or can be prepared according to a known method.

The step (9-2) is usually carried out in a solvent. Examples of the solvent to be used in the reaction include hydrocarbons, ethers, amides and mixed solvents of these two or more solvents.

In the reaction, N,N-dimethylhydrazine is usually used within a range of 0.8 to 5 molar ratios as opposed to 1 mole of the compound (B22).

The reaction period of the reaction is usually within a range of 5 minutes to 72 hours. The reaction temperature is usually within a range of −20 to 100° C.

When the reaction is completed, the precipitated out-solids are collected by a filtration, or water is added to the reaction mixture, and the resulting mixture was extracted with organic solvent(s), and the organic layers are worked up (for example, drying and concentration) to obtain the compound (B23).

### Reference Process 10

A compound represented by formula (B25) (hereinafter, referred to as “Compound (B25)”) can be prepared by a step where the compound (B23) and triphosgene are reacted to obtain a compound represented by formula (B24) (hereinafter, referred to as “Compound (B24)”) (hereinafter, referred to as “Step (10-1)”) and a step where the compound (B24) and a compound represented by formula (M17) (hereinafter, referred to as “Compound (M17)”) are reacted in the presence of a base (hereinafter, referred to “Step (10-2)”).

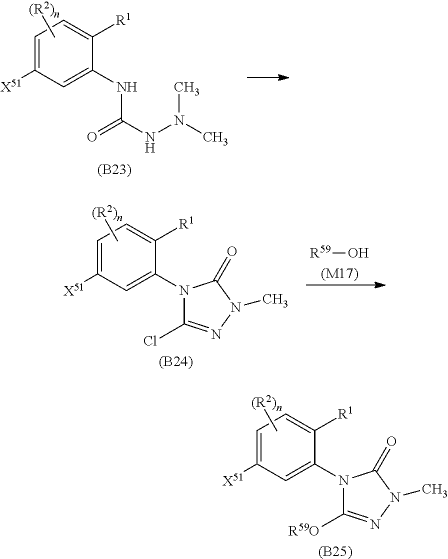

[wherein R⁵⁹ represents a C1-C3 alkyl group which may be optionally substituted with one or more halogen atoms; and the other symbols are the same as defined above.]

The step (10-1) is usually carried out in a solvent. Examples of the solvents to be used in the reaction include hydrocarbons, ethers, halogenated hydrocarbons and mixed solvents of these two or more solvents.

In the reaction, triphosgene is usually carried out within a range of 1 to 10 molar ratio(s) as opposed to 1 mole of the compound (B23).

The reaction period of the reaction is usually within a range of 5 minutes to 72 hours. The reaction temperature is usually within a range of 0 to 150° C.

When the reaction is completed, water is added to the reaction mixtures, and the resulting mixtures are extracted with organic solvent(s), and the organic layers are worked up (such as concentration and drying) to isolate the compound (B24).

The step (10-2) is usually carried out in a solvent. Examples of the solvent to be used in the reaction include hydrocarbons, ethers, halogenated hydrocarbons, amides, esters, nitriles and mixed solvents of these two or more solvents.

Examples of the base to be used in the reaction include organic bases, alkali metal carbonates, alkali metal hydrocarbonates, sodium hydride, and mixed bases of these two or more bases.

In the reaction, the compound (M17) is usually used within a range of 1 to 10 molar ratio(s), and the base is usually used within a range of 1 to 20 molar ratio(s), as opposed to 1 mole of the compound (B24).

The reaction temperature is usually within a range of −20 to 100° C. The reaction period of the reaction is usually within a range of 0.1 to 48 hours.

When the reaction is completed, water is added to the reaction mixtures, and the resulting mixtures are extracted with organic solvent(s), and the organic layers are worked up (such as concentration and drying) to isolate the compound (B25).

### Reference Process 11

A compound represented by formula (B35) (hereinafter, referred to as “Compound (B35)”) can be prepared by a step where a compound represented by formula (B33) (hereinafter, referred to as “Compound (B33)”) and hydroxylamine are reacted in the presence of a base to obtain a compound represented by formula (B34) (hereinafter, referred to as “Compound (B34)”) (hereinafter, referred to as “Step (11-1)”) and a step where the compound (B34) and a compound represented by formula (M18) (hereinafter, referred to as “Compound (M17)”) are reacted in the presence of a base (hereinafter, referred to “Step (11-2)”).

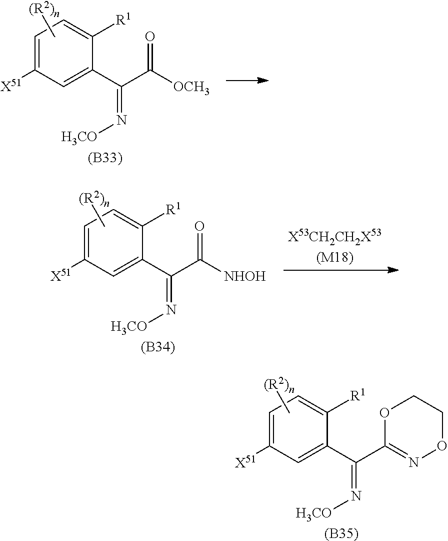

[wherein the symbols are the same as defined above.]

The step (11-1) and the step (11-2) can be carried out according to the Process O.

The compound (B33) is a known compound, or can be prepared according to a known method.

### Reference Process 12

A compound represented by formula (B39) (hereinafter, referred to as “Compound (B39)”) can be prepared by a step where the compound (B22) and an azide agent are reacted to obtain a compound represented by formula (B38) (hereinafter, referred to as “Compound (B38)”) (hereinafter, referred to as “Step (12-1)”) and a step where the compound (B38) and the compound (M13) are reacted in the presence of a base (hereinafter, referred to “Step (12-2)”).

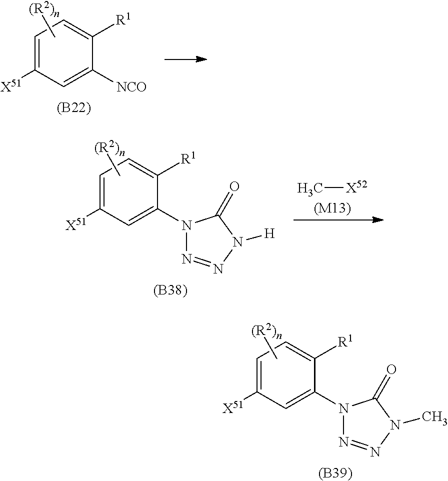

[wherein the symbols are the same as defined above.]

The step (12-1) and the step (12-2) can be carried out according to the Process P.

### Reference Process 13

A compound represented by formula (B42) (hereinafter, referred to as “Compound (B42)”) can be prepared by a step where a compound represented by formula (B40) (hereinafter, referred to as “Compound (B40)”) and the compound (M16) are reacted to obtain a compound represented by formula (B41) (hereinafter, referred to as “Compound (B41)”) (hereinafter, referred to as “Step (13-1)”) and a step where the compound (B41) and a phosgene are reacted in the presence of a base (hereinafter, referred to “Step (13-2)”).

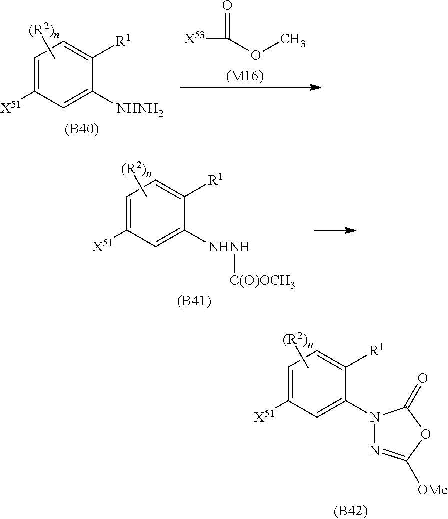

[wherein the symbols are the same as defined above.]

The step (B13-1) and the step (13-2) can be carried out according to the Process Q.

The compound (B40) is a known compound, or can be prepared according to a known method.

### Reference Process 14

A compound represented by formula (B47) (hereinafter, referred to as “Compound (B47)”) can be prepared by reacting a compound represented by formula (B46) (hereinafter, referred to as “Compound (B46)”) in the presence of a hydrazine.

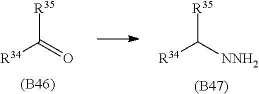

[wherein the symbols are the same as defined above.]

The reaction can be carried out according to a method described in Tetrahedron Letters, 2011, 52, 1553.

The compound (B46) is a known compound, or can be prepared according to a known method.

### Reference Process 15

A compound represented by formula (B49) (hereinafter, referred to as “Compound (B49)”) can be prepared by reacting a compound represented by formula (B48) (hereinafter, referred to as “Compound (B48)”) with the compound (M19) in the presence of a catalyst and a base.

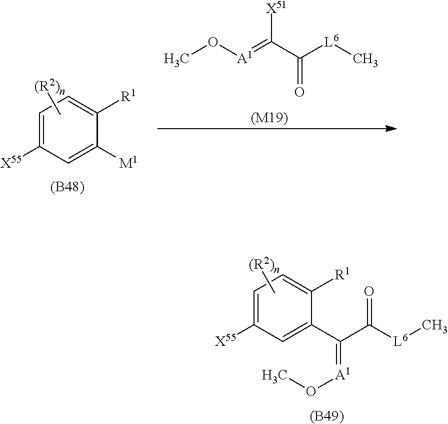

[wherein, X⁵⁵ represents a leaving group (such as a chlorine atom, bromine atom, iodine atom, and triflyloxy group), B(OH)₂, or 4,4,5,5-tetramethyl-1,3,2-dioxaborolan-2-yl group; and the other symbols are the same as defined above.]

The reaction can be carried out according to the Process U.

The compound (B48) is known compounds, or can be prepared according to a known method.

### Reference Process 16

The compound (B50) can be prepared by reacting the compound (B44) with bis(pinacolato)diboron in the presence of a base and a palladium catalyst.

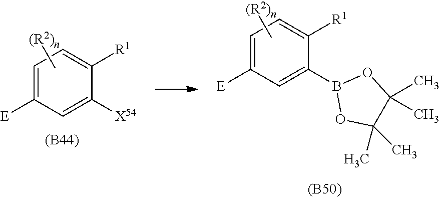

[wherein the symbols are the same as defined above.]

The reaction can be carried out according to the Reference Process 3.

### Process a

A compound represented by formula (a1) can be prepared by reacting the compound (B6) with a compound represented by formula (m1) (hereinafter, referred to as “Compound (m1)”) in the presence of a base.

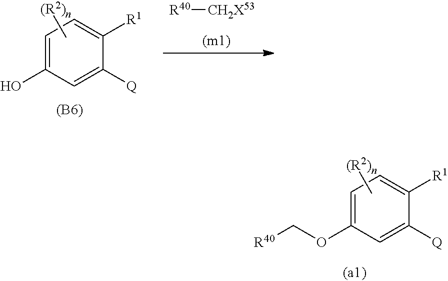

[wherein the symbols are the same as defined above.]

The reaction can be carried out by using the compound (m1) in place of the compound (M11) according to the Process K.

The compound (m1) is a known compound, or can be prepared according to a known method.

### Process b1

A compound represented by formula (a2) can be prepared by reacting the compound (B19) with a compound represented by formula (m2) (hereinafter, referred to as “Compound (m2)”) in the presence of a base.

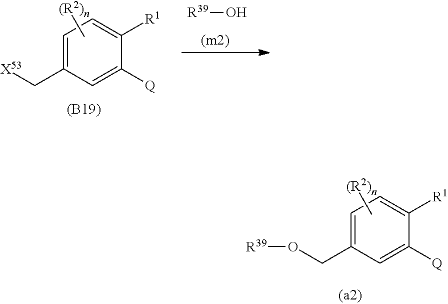

[wherein the symbols are the same as defined above.]

The reaction can be carried out by using the compound (B19) in place of the compound (M11) and using the compound (m2) in place of the compound (B6) according to the Process K.

The compound (m2) is a known compound, or can be prepared according to a known method.

### Process b2

A compound represented by formula (a3) can be prepared by reacting the compound (B19) with a compound represented by formula (m3) (hereinafter, referred to as “Compound (m3)” in the presence of a base.

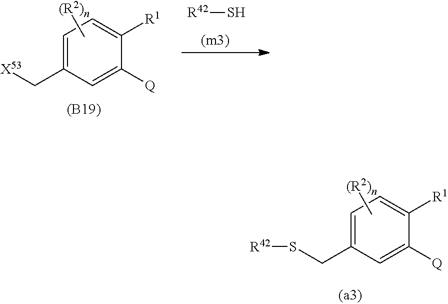

[wherein the symbols are the same as defined above.]

The reaction can be carried out by using the compound (B19) in place of the compound (M11) and using the compound (m3) in place of the compound (B6) according to the Process K.

The compound (m3) is a known compound, or can be prepared according to a known method.

### Process k

A compound represented by formula (a8) can be prepared by reacting a compound represented by formula (b6) (hereinafter, referred to as “Compound (b6)”) with a compound represented by formula (m11) (hereinafter, referred to as “Compound (m11)”) in the presence of a base.

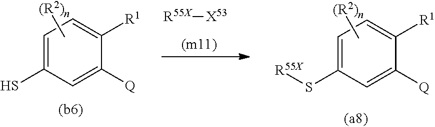

[wherein R^{55X} represents R⁴¹ or R⁴³CH₂—, and the other symbols are the same as defined above.]

The reaction can be carried out by using the compound (b6) in place of the compound (B6) and using the compound (m11) in place of the compound (M11) according to the Process K.

The compound (m11) is a known compound, or can be prepared according to a known method.

### Process 1

A compound represented by formula (a9) can be prepared by a step where a compound represented by formula (b7) (hereinafter, referred to as “Compound (b7)”) and the compound (M12) are reacted in the presence of a base to obtain a compound represented by formula (b8) (hereinafter, referred to as “Compound (b8)”) (hereinafter, referred to as “Step (1-1)”) and a step where the compound (b8) and the compound (M13) are reacted in the presence of a base (hereinafter, referred to “Step (1-2)”).

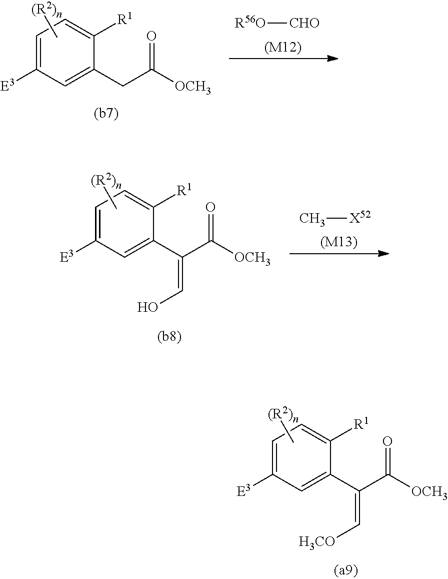

[wherein the symbols are the same defined above.]

The step (1-1) can be carried out by using the compound (b7) in place of the compound (B7) and using the compound (m12) in place of the compound (M12) according to the step (1-1) of the process L.

The step (1-2) can be carried out by using the compound (b8) in place of the compound (B8) and using the compound (m13) in place of the compound (M13) according to the step (1-2) of the Process L.

The compound (b7) is a known compound, or can be prepared according to a known method.

### Process m

A compound represented by formula (a10) can be prepared by a step where a compound represented by formula (b43) (hereinafter, referred to as “Compound (b43)”) and the compound (M14) are reacted in the presence of a base to obtain a compound represented by formula (b9) (hereinafter, referred to as “Compound (b9)”) (hereinafter, referred to as “Step (m-1)”) and a step where the compound (b9) and the compound (M13) are reacted in the presence of a base (hereinafter, referred to “Step (m-2)”).

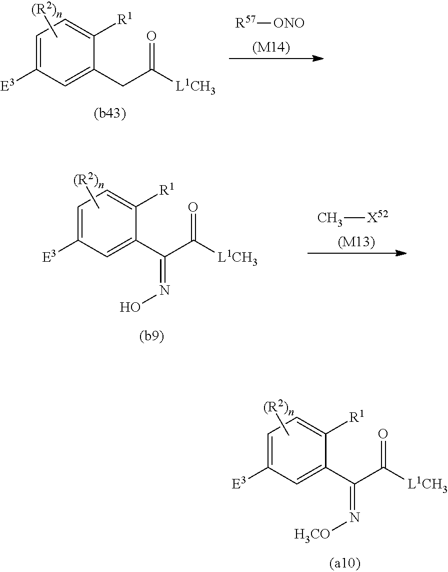

[wherein the symbols are the same as defined above.]

The step (m-1) can be carried out by using the compound (b43) in place of the compound (B43) according to the step (M-1) of the Process M.

The step (m-2) can be carried out by using the compound (b9) in place of the compound (B9) according to the step (M-2) of the Process M.

The compound (b43) is a commercially available compound, or can be prepared according to a known method.

### Process n

A compound represented by formula (a12) can be prepared by reacting a compound represented by formula (a11) (hereinafter, referred to as “Compound (a11)”) with methyl amine.

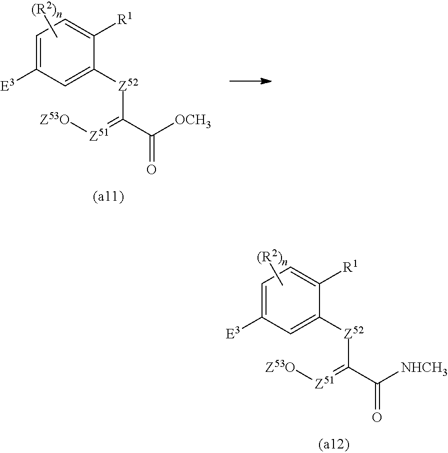

[wherein the symbols are the same as defined above.]

The reaction can be carried out by using the compound (a11) in place of the compound (A11) according to the Process N.

### Process o

A compound represented by formula (a14) can be prepared by a step where a compound represented by formula (a13) (hereinafter, referred to as “Compound (a13)”) and hydroxyl amine are reacted in the presence of a base to obtain a compound represented by formula (b26) (hereinafter, referred to as “Compound (b26)”) (hereinafter, referred to as “Step (o-1)”) and a step where the compound (b26) and the compound (M18) are reacted in the presence of a base (hereinafter, referred to “Step (o-2)”).

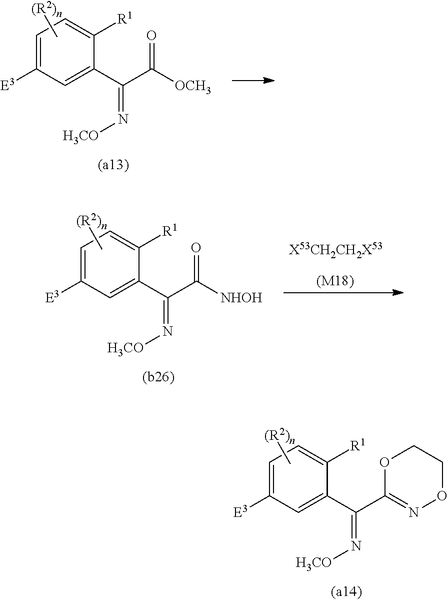

[wherein the symbols are the same as defined above.]

The step (o-1) can be carried out by using the compound (a13) in place of the compound (A13) according to the step (o-1) of the Process O.

The step (o-2) can be carried out by using the compound (b26) in place of the compound (B26) according to the step (o-2) of the Process O.

### Process p

A compound represented by formula (a15) can be prepared by a step where a compound represented by formula (b27) (hereinafter, referred to as “Compound (b27)”) and an isocyanate agent are reacted to obtain a compound represented by formula (b28) (hereinafter, referred to as “Compound (b28)”) (hereinafter, referred to as “Step (p-1)”), a step where the compound (b28) and an azide compound are reacted to obtain a compound represented by formula (b29) (hereinafter, referred to as “Compound (b29)”) (hereinafter, referred to “Step (p-2)”), and a step where the compound (b29) and the compound (M13) are reacted in the presence of a base (hereinafter, referred to “Step (p-3)”).

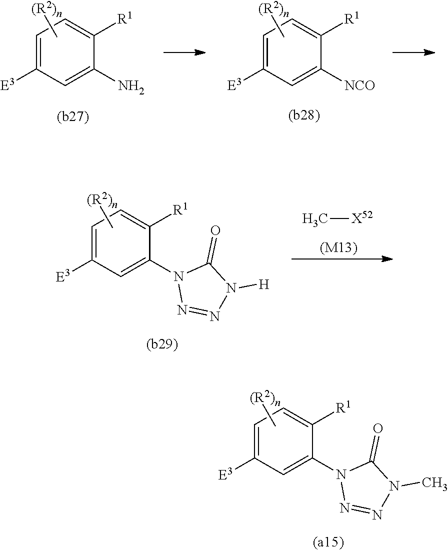

[wherein the symbols are the same as defined above.]

The step (p-1) can be carried out by using the compound (b27) in place of the compound (B27) according to the step (P-1) of the Process P.

The step (p-2) can be carried out by using the compound (b28) in place of the compound (B28) according to the step (P-2) of the Process P.

The step (p-3) can be carried out by using the compound (b29) in place of the compound (B29) according to the step (P-3) of the Process P.

The compound (b27) is a commercially available compound, or can be prepared according to a known method.

### Process q

A compound represented by formula (a16) (hereinafter, referred to as “Compound (b16)”) can be prepared by a step where a compound represented by formula (b44) (hereinafter, referred to as “Compound (b44)”) and hydrazine are reacted to obtain a compound represented by formula (b30) (hereinafter, referred to as “Compound (b30)”) (hereinafter, referred to as “Step (q-1)”), a step where the compound (b30) and the compound (M16) are reacted to obtain a compound represented by formula (b31) (hereinafter, referred to as “Compound (b31)”) (hereinafter, referred to “Step (q-2)”), and a step where the compound (b31) and a phosgene are reacted in the presence of a base (hereinafter, referred to “Step (q-3)”).

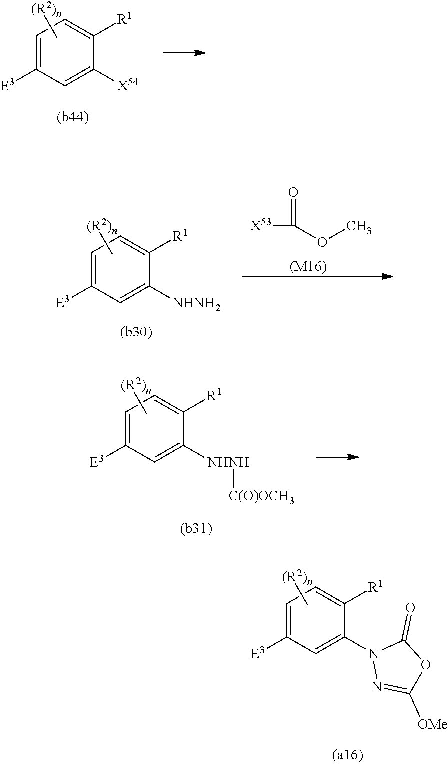

[wherein the symbols are the same as defined above.]

The step (q-1) can be carried out by using the compound (b44) in place of the compound (B44) according to the step (Q-1) of the Process Q.

The step (q-2) can be carried out by using the compound (b30) in place of the compound (B30) according to the step (Q-2) of the Process Q.

The step (q-3) can be carried out by using the compound (b31) in place of the compound (B31) according to the step (Q-3) of the Process Q.

The compound (b44) is a commercially available compound, or can be prepared according to a known method.

Also the compound (a16) can be prepared according to a method described in JP 2006-151865 A1 or JP 2006-160620 A1.

### Process s

A N-oxide of the compound represented by formula (III) can be prepared by reacting a compound represented by formula (III) with an oxidizing agent. The reaction can be carried out according to a method described in US patent publication No. 2018/0009778 A1 or WO2016/121970 A1.

### Process t

A compound represented by formula (a17) (hereinafter, referred to as “Compound (a17)”) can be prepared by reacting a compound represented by formula (b32) (hereinafter, referred to as “Compound (b32)”) with a compound represented by formula (m19) (hereinafter, referred to as “Compound (bm19)”) in the presence of a phosphines and an azodiester

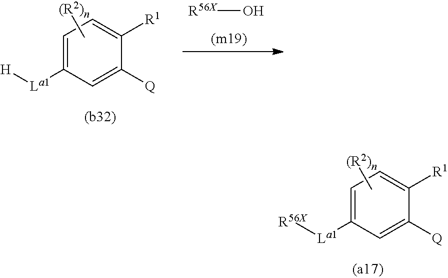

[wherein, a combination of Lai and R^{56X} represents a combination where Lai represents an oxygen atom, R^{56X} represents a C5-C6 a cycloalkyl group which may be optionally substituted with one or more substituents selected from Group T, a combination where L^{a1} represents a sulfur atom, R^{56x} represents a C3-C6 cycloalkyl group which may be optionally substituted with one or more substituents selected from Group T; and the other symbols are the same as defined above.]

The reaction is usually carried out in a solvent. Examples of the solvent to be used in the reaction include hydrocarbons, ethers, halogenated hydrocarbons, amides, esters, nitriles, and mixed solvents of these two or more solvents.

Examples of the phosphines include triphenylphosphine and trimethylphosphine.

Examples of the azodiesters include diethyl azodicarboxylate, diisopropyl azodicarboxylate, and bis(2-methoxyethyl) azodicarboxylate.

In the reaction, the compound (m19) is usually used within a range of 1 to 10 molar ratio(s), the phosphines is usually used within a range of 1 to 10 molar ratio(s), and the azodiester is usually used within a range of 1 to 10 molar ratio(s), as opposed to 1 mole of the compound (b32).

The reaction temperature is usually within a range of 0 to 150° C. The reaction period of the reaction is usually within a range of 0.1 to 48 hours.

When the reaction is completed, water is added to the reaction mixtures, and the resulting mixtures are extracted with organic solvent(s), and the organic layers are worked up (such as concentration and drying) to isolate the compound (a17).

The compound (m19) is a known compound, or can be prepared according to a known method.

### Reference Process 1a

The compound (b6) can be prepared by reacting the compound (B6) with a sulfurizing agent.

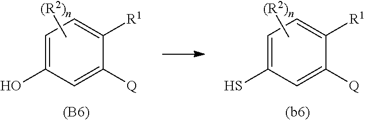

[wherein the symbols are the same as defined above.]

The reaction can be carried out according to a method described in US patent Publication No. 2014/135359 A1.

The present compound C may be mixed or combined with one or more ingredients selected from a group consisting of the following Group (a), Group (b), Group (c), and Group (d), (hereinafter, referred to as “Present ingredient”).

The above-mentioned mixing or combining represents a use of the present compound C and the Present ingredient at same time, separately or at certain intervals.

When the present compound C and the present ingredient are used at the same time, the compound of the present invention and the Present ingredient may be contained in separate formulations respectively, or may be contained in the same one formulation.

One aspect of the present invention is a composition comprising one or more ingredients selected from Group (a), Group (b), Group (c) and Group (d) as well as the present compound C (hereinafter, referred to as Composition A).

Group (a) is a group consisting of each active ingredient as Acetylcholinesterase inhibitors (for example, carbamate insecticides, or organophosphorus insecticides), GABA-gated chloride channel blockers (for example, phenylpyrazole insecticides), Sodium channel modulators (for example, pyrethroid insecticides), Nicotinic acetylcholine receptor competitive modulators (for example, neonicotinoid insecticides), Nicotinic acetylcholine receptor allosteric modulators, Glutamatergic chlorine ion channel allosteric modulators (for example, macrolide insecticides), Juvenile hormone mimic, Multisite inhibitors, chordotonal organ TRPV channel modulators, Mites growth inhibitors, Mitochondria ATP biosynthetic enzyme inhibitors, Uncouplers of oxidative phosphorylation, Nicotinic acetylcholine receptor channel blocker (for example, Nereistoxin insecticides), Chitin synthesis inhibitors, Molting inhibitors, Ecdysone receptor agonist, Octopamine receptor agonist, Inhibitors of Mitochondrial electron transport system complex I, II, III and IV, Voltage-dependent sodium channel blockers, Acetyl CoA carboxylase inhibitor, Ryanodine receptor modulator (for example, Diamide insecticides), Chordotonal organ modulators, Microbial pesticides; and
the other insecticidal, miticidal or nematicidal active ingredients.

These ingredients are classified as a class based on the action mechanism of IRAC.

Group (b) is a group consisting of
Nucleic acid synthesis inhibitors (for example, Phenylamide fungicides, or Acylamino acid fungicides), cell division and cytoskeleton inhibitors (for example, MBC fungicides), Respiratory inhibitors (for example, QoI fungicides or Qil fungicides), Amino acid synthesis and protein synthesis inhibitors (for example, anilinopyridine fungicides), Signal transduction inhibitors, Lipid synthesis and membrane synthesis inhibitors, sterol biosynthesis inhibitors (for example, DMI fungicides such as triazole), cell wall synthesis inhibitors, Melanin synthesis inhibitors, Plant defense inducers, Other action point contact active fungicides, Microbial fungicides, and the other fungicidal ingredients. These are classified as a class based on the action mechanism of FRAC.

Group (c) is a plant growth modulating ingredient group (including Mycorrhizal fungi, and Root nodule bacteria).

Group (d) is a repellent ingredient group consisting of a bird repellant ingredient and an insect repellant ingredient.

Examples of the combination of the Present ingredient and the present compound C are described below. For example, alanycarb+SX represents a combination of alanycarb and SX. The symbol of “SX” represents any one of the compound of the present invention selected from the Compound Class SX944 to the Compound Class SX1453. Also, all of the below-mentioned present active ingredient are known ingredients, and are commercially available or may be produced by the known method. If the present ingredient is a bacterium, it is available from the bacterial authority depository. The numerical number in bracket represents a CAS RN (Register Trademark).

Combinations of the present ingredient of the above Group (a) and the present compound C:
abamectin+SX, acephate+SX, acequinocyl+SX, acetamiprid+SX, acetoprole+SX, acrinathrin+SX, acynonapyr+SX, afidopyropen+SX, afoxolaner+SX, alanycarb+SX, aldicarb+SX, allethrin+SX, alpha-cypermethrin+SX, alpha-endosulfan+SX, aluminium phosphide+SX, amitraz+SX, azadirachtin+SX, azamethiphos+SX, azinphos-ethyl+SX, azinphos-methyl+SX, azocyclotin+SX, bark of Celastrus angulatus+SX, bendiocarb+SX, benfluthrin+SX, benfuracarb+SX, bensultap+SX, benzoximate+SX, benzpyrimoxan+SX, beta-cyfluthrin+SX, beta-cypermethrin+SX, bifenazate+SX, bifenthrin+SX, bioallethrin+SX, bioresmethrin+SX, bistrifluron+SX, borax+SX, boric acid+SX, broflanilide+SX, bromopropylate+SX, buprofezin+SX, butocarboxim+SX, butoxycarboxim+SX, cadusafos+SX, calcium phosphide+SX, carbaryl+SX, carbofuran+SX, carbosulfan+SX, cartap hydrochloride+SX, cartap+SX, chinomethionat+SX, chlorantraniliprole+SX, chlordane+SX, chlorethoxyfos+SX, chlorfenapyr+SX, chlorfenvinphos+SX, chlorfluazuron+SX, chlormephos+SX, chloropicrin+SX, chlorpyrifos+SX, chlorpyrifos-methyl+SX, chromafenozide+SX, clofentezine+SX, clothianidin+SX, concanamycin A+SX, coumaphos+SX, cryolite+SX, cyanophos+SX, cyantraniliprole+SX, cyclaniliprole+SX, cyclobutrifluram+SX, cycloprothrin+SX, cycloxaprid+SX, cyenopyrafen+SX, cyetpyrafen+SX, cyflumetofen+SX, cyfluthrin+SX, cyhalodiamide+SX, cyhalothrin+SX, cyhexatin+SX, cypermethrin+SX, cyphenothrin+SX, cyromazine+SX, dazomet+SX, deltamethrin+SX, demeton-S-methyl+SX, diafenthiuron+SX, diazinon+SX, dichlorvos+SX, dicloromezotiaz+SX, dicofol+SX, dicrotophos+SX, diflovidazin+SX, diflubenzuron+SX, dimefluthrin+SX, dimethoate+SX, dimethylvinphos+SX, dimpropyridaz+SX, dinotefuran+SX, disodium octaborate+SX, disulfoton+SX, DNOC(2-methyl-4,6-dinitrophenol)+SX, doramectin+SX, dried leaves of Dryopteris filix-mas+SX, emamectin-benzoate+SX, empenthrin+SX, endosulfan+SX, EPN(O-ethyl O-(4-nitrophenyl) phenylphosphonothioate)+SX, epsilon-metofluthrin+SX, epsilon-momfluorothrin+SX, esfenvalerate+SX, ethiofencarb+SX, ethion+SX, ethiprole+SX, ethoprophos+SX, etofenprox+SX, etoxazole+SX, extract of Artemisia absinthium+SX, extract of Cassia nigricans+SX, extract of clitoria ternatea+SX, extract of Symphytum officinale+SX, extracts or simulated blend of Chenopodium ambrosioides+SX, extract of Tanacetum vulgare+SX, extract of Urtica dioica+SX, extract of Viscum album+SX, famphur+SX, fenamiphos+SX, fenazaquin+SX, fenbutatin oxide+SX, fenitrothion+SX, fenobucarb+SX, fenoxycarb+SX, fenpropathrin+SX, fenpyroximate+SX, fenthion+SX, fenvalerate+SX, fipronil+SX, flometoquin+SX, flonicamid+SX, fluacrypyrim+SX, fluazaindolizine+SX, fluazuron+SX, flubendiamide+SX, flucycloxuron+SX, flucythrinate+SX, fluensulfone+SX, flufenoprox+SX, flufenoxuron+SX, flufiprole+SX, flumethrin+SX, flupentiofenox+SX, flupyradifurone+SX, flupyrimin+SX, fluralaner+SX, fluvalinate+SX, fluxametamide+SX, formetanate+SX, fosthiazate+SX, furamethrin+SX, furathiocarb+SX, gamma-cyhalothrin+SX, GS-omega/kappa HXTX-Hvla peptide+SX, halfenprox+SX, halofenozide+SX, heptafluthrin+SX, heptenophos+SX, hexaflumuron+SX, hexythiazox+SX, potassium salt of hop beta acid+SX, hydramethylnon+SX, hydroprene+SX, imicyafos+SX, imidacloprid+SX, imidaclothiz+SX, imiprothrin+SX, indoxacarb+SX, isocycloseram+SX, isofenphos+SX, isoprocarb+SX, isopropyl-O-(methoxyaminothiophosphoryl) salicylate+SX, isoxathion+SX, ivermectin+SX, kadethrin+SX, kappa-tefluthrin+SX, kappa-bifenthrin+SX, kinoprene+SX, lambda-cyhalothrin+SX, lenoremycin+SX, lepimectin+SX, lime sulfur+SX, lotilaner+SX, lufenuron+SX, machine oil+SX, malathion+SX, mecarbam+SX, meperfluthrin+SX, metaflumizone+SX, metam+SX, methamidophos+SX, methidathion+SX, methiocarb+SX, methomyl+SX, methoprene+SX, methoxychlor+SX, methoxyfenozide+SX, methyl bromide+SX, metofluthrin+SX, metolcarb+SX, metoxadiazone+SX, mevinphos+SX, milbemectin+SX, milbemycin oxime+SX, momfluorothrin+SX, monocrotophos+SX, moxidectin+SX, naled+SX, neem oil+SX, nicotine+SX, nicotine-sulfate+SX, nitenpyram+SX, novaluron+SX, noviflumuron+SX, oil of the seeds of Chenopodium anthelminticum+SX, omethoate+SX, oxamyl+SX, oxazosulfyl+SX, oxydemeton-methyl+SX, parathion+SX, parathion-methyl+SX, permethrin+SX, phenothrin+SX, phenthoate+SX, phorate+SX, phosalone+SX, phosmet+SX, phosphamidon+SX, phosphine+SX, phoxim+SX, pirimicarb+SX, pirimiphos-methyl+SX, prallethrin+SX, profenofos+SX, profluthrin+SX, propargite+SX, propetamphos+SX, propoxur+SX, propylene glycol alginate+SX, prothiofos+SX, pyflubumide+SX, pymetrozine+SX, pyraclofos+SX, pyrethrins+SX, pyridaben+SX, pyridalyl+SX, pyridaphenthion+SX, pyrifluquinazone+SX, pyrimidifen+SX, pyriminostrobin+SX, pyriprole+SX, pyriproxyfen+SX, quinalphos+SX, resmethrin+SX, rotenone+SX, ryanodine+SX, sarolaner+SX, selamectin+SX, sigma-cypermethrin+SX, silafluofen+SX, sodium borate+SX, sodium metaborate+SX, spinetoram+SX, spinosad+SX, spirodiclofen+SX, spiromesifen+SX, spiropidion+SX, spirotetramat+SX, sulfluramid+SX, sulfotep+SX, sulfoxaflor+SX, sulfur+SX, sulfuryl fluoride+SX, tartar emetic+SX, tau-fluvalinate+SX, tebufenozide+SX, tebufenpyrad+SX, tebupirimfos+SX, teflubenzuron+SX, tefluthrin+SX, temephos+SX, terbufos+SX, terpene constituents of the extract of Chenopodium ambrosioides near ambrosioides+SX, tetrachlorantraniliprole+SX, tetrachlorvinphos+SX, tetradifon+SX, tetramethrin+SX, tetramethylfluthrin+SX, tetraniliprole+SX, theta-cypermethrin+SX, thiacloprid+SX, thiamethoxam+SX, thiocyclam+SX, thiodicarb+SX, thiofanox+SX, thiometon+SX, thiosultap-disodium+SX, thiosultap-monosodium+SX, tioxazafen+SX, tolfenpyrad+SX, tralomethrin+SX, transfluthrin+SX, triazamate+SX, triazophos+SX, trichlorfon+SX, triflumezopyrim+SX, triflumuron+SX, trimethacarb+SX, tyclopyrazoflor+SX, vamidothion+SX, wood extract of Quassia amara+SX, XMC (3,5-dimethylphenyl N-methylcarbamate)+SX, xylylcarb+SX, zeta-cypermethrin+SX, zinc phosphide+SX, 4-[5-(3,5-dichlorophenyl)-5-(trifluoromethyl)-4,5-dihydro-1,2-oxazol-3-yl]-2-methyl-N-(1-oxothietan-3-yl)benzamide (1241050-20-3)+SX, 3-methoxy-N-(5-{5-(trifluoromethyl)-5-[3-(trifluoromethyl)phenyl]-4,5-dihydro-1,2-oxazol-3-yl}indan-1-yl)propanamide (1118626-57-5)+SX, 4-chloro-5-[2,2-difluoro-2-(3,4,5-trifluorophenyl)ethoxy]-2-methylphenyl 2,2,2-trifluoroethyl sulfoxide (1632218-00-8)+SX, 4-fluoro-5-[2,2-difluoro-2-(3,4,5-trifluorophenyl)ethoxy]-2-methylphenyl 2,2,2-trifluoroethyl sulfoxide (1632217-98-1)+SX, 2-({2-fluoro-4-methyl-5-[(2,2,2-trifluoroethyl)sulfinyl]phenyl}imino)-3-(2,2,2-trifluoroethyl)-1,3-thiazolidin-4-one (1445683-71-5)+SX, (3R)-3-(2-chlorothiazol-5-yl)-8-methyl-7-oxo-6-phenyl-2,3-dihydrothiazolo[3,2-a]pyrimidin-4-ium-5-olate (2249718-27-0)+SX, BT crop protein Cry1Ab+SX, BT crop protein Cry1Ac+SX, BT crop protein Cry1Fa+SX, BT crop protein Cry1A.105+SX, BT crop protein Cry2Ab+SX, BT crop protein Vip3A+SX, BT crop protein Cry3A+SX, BT crop protein Cry3Ab+SX, BT crop protein Cry3Bb+SX, BT crop protein Cry34Ab1/Cry35Ab1+SX, Adoxophyes orana granulosis virus strain BV-0001+SX, Anticarsia gemmatalis mNPV+SX, Autographa californica mNPV+SX, Cydia pomonella GV strain V15+SX, Cydia pomonella GV strain V22+SX, Cryptophlebia leucotreta GV+SX, Dendrolimus punctatus cypovirus+SX, Helicoverpa armigera NPV strain BV-0003+SX, Helicoverpa zea NPV+SX, Lymantria dispar NPV+SX, Mamestra brassicae NPV+SX, Mamestra configurata NPV+SX, Neodiprion abietis NPV+SX, Neodiprion lecontei NPV+SX, Neodiprion sertifer NPV+SX, Nosema locustae+SX, Orgyia pseudotsugata NPV+SX, Pieris rapae GV+SX, Plodia interpunctella GV+SX, Spodoptera exigua mNPV+SX, Spodoptera littoralis mNPV+SX, Spodoptera litura NPV+SX, Arthrobotrys dactyloides+SX, Bacillus firmus strain GB-126+SX, Bacillus firmus strain 1-1582+SX, Bacillus megaterium+SX, Bacillus sp. strain AQ175+SX, Bacillus sp. strain AQ177+SX, Bacillus sp. strain AQ178+SX, Bacillus sphaericus strain 2362+SX, Bacillus sphaericus strain ABTS1743+SX, Bacillus sphaericus Serotype strain H5a5b+SX, Bacillus thuringiensis strain AQ52+SX, Bacillus thuringiensis strain BD #32+SX, Bacillus thuringiensis strain CR-371+SX, Bacillus thuringiensis subsp. Aizawai strain ABTS-1857+SX, Bacillus thuringiensis subsp. Aizawai strain AM65-52+SX, Bacillus thuringiensis subsp. Aizawai strain GC-91+SX, Bacillus thuringiensis subsp. Aizawai Serotype strain H-7+SX, Bacillus thuringiensis subsp. Kurstaki strain ABTS351+SX, Bacillus thuringiensis subsp. Kurstaki strain BMP123+SX, Bacillus thuringiensis subsp. Kurstaki strain EG234+SX, Bacillus thuringiensis subsp. Kurstaki strain EG7841+SX, Bacillus thuringiensis subsp. Kurstaki strain EVB113-19+SX, Bacillus thuringiensis subsp. Kurstaki strain F810+SX, Bacillus thuringiensis subsp. Kurstaki strain HD-1+SX, Bacillus thuringiensis subsp. Kurstaki strain PB54+SX, Bacillus thuringiensis subsp. Kurstaki strain SA-11+SX, Bacillus thuringiensis subsp. Kurstaki strain SA-12+SX, Bacillus thuringiensis subsp. Tenebriosis strain NB176+SX, Bacillus thuringiensis subsp. Thuringiensis strain MPPL002+SX, Bacillus thuringiensis subsp. morrisoni+SX, Bacillus thuringiensis var. colmeri+SX, Bacillus thuringiensis var. darmstadiensis strain 24-91+SX, Bacillus thuringiensis var. dendrolimus+SX, Bacillus thuringiensis var. galleriae+SX, Bacillus thuringiensis var. israelensis strain BMP144+SX, Bacillus thuringiensis var. israelensis serotype strain H-14+SX, Bacillus thuringiensis var. japonensis strain buibui+SX, Bacillus thuringiensis var. san diego strain M-7+SX, Bacillus thuringiensis var. 7216+SX, Bacillus thuringiensis var. aegypti+SX, Bacillus thuringiensis var. T36+SX, Beauveria bassiana strain ANT-03+SX, Beauveria bassiana strain ATCC74040+SX, Beauveria bassiana strain GHA+SX, Beauveria brongniartii+SX, Burkholderia rinojensis strain A396+SX, Chromobacterium subtsugae strain PRAA4-1T+SX, Dactyllela ellipsospora+SX, Dectylaria thaumasia+SX, Hirsutella minnesotensis+SX, Hirsutella rhossiliensis+SX, Hirsutella thompsonii+SX, Lagenidium giganteum+SX, Lecanicillium lecanii strain KV01+SX, Lecanicillium lecanii conidia of strain DAOM198499+SX, Lecanicillium lecanii conidia of strain DAOM216596+SX, Lecanicillium muscarium strain Ve6+SX, Metarhizium anisopliae strain F52+SX, Metarhizium anisopliae var. acridum+SX, Metarhizium anisopliae var. anisopliae BIPESCO 5/F52+SX, Metarhizium flavoviride+SX, Monacrosporium phymatopagum+SX, Paecilomyces fumosoroseus Apopka strain 97+SX, Paecilomyces lilacinus strain 251+SX, Paecilomyces tenuipes strain T1+SX, Paenibacillus popilliae+SX, Pasteuria nishizawae strain Pn1+SX, Pasteuria penetrans+SX, Pasteuria usgae+SX, Pasteuria thoynei+SX, Serratia entomophila+SX, Verticillium chlamydosporium+SX, Verticillium lecani strain NCIM1312+SX, 2-chloro-N-cyclopropyl-5-{l-[2,6-dichloro-4-(1,1,1,2,3,3,3-heptafluoropropan-2-yl)phenyl]-1H-pyrazol-4-yl}-N-methylpyridine-3-carboxamide (1771741-86-6)+SX, N-{4-chloro-3-[(1-cyanocyclopropyl)carbamoyl]phenyl}-1-methyl-4-(methanesulfonyl)-3-(1,1,2,2,2-pentafluoroethyl)-1H-pyrazole-3-carboxamide (1400768-21-9)+SX, 11-(4-chloro-2,6-dimethylphenyl)-12-hydroxy-1,4-dioxa-9-azadispiro[4.2.4.2]tetradec-11-en-10-one (907187-07-9)+SX, 3-(4′-fluoro-2,4-dimethyl[1,1′-biphenyl]-3-yl)-4-hydroxy-8-oxa-1-azaspiro[4.5]dec-3-en-2-one (1031385-91-7)+SX, N-[3-chloro-1-(pyridin-3-yl)-1H-pyrazol-4-yl]-2-(methanesulfonyl)propanamide (2396747-83-2)+SX, 2-isopropyl-5-[(3,4,4-trifluoro-3-buten-1-yl)sulfonyl]-1,3,4-thiadiazole (2058052-95-0)+SX, 1,4-dimethyl-2-[2-(3-pyridinyl)-2H-indazol-5-yl]-1,2,4-triazolidine-3,5-dione (2171099-09-3)+SX, cyproflanilide+SX, N-({2-fluoro-4-[(2S,3S)-2-hydroxy-3-(3,4,5-trichlorophenyl)-3-(trifluoromethyl)pyrrolidin-1-yl]phenyl}methyl)cyclopropanecarboxamide+SX.

Combinations of the present ingredient of the above Group (b) and the present compound C:
acibenzolar-S-methyl+SX, aldimorph+SX, ametoctradin+SX, aminopyrifen+SX, amisulbrom+SX, anilazine+SX, azaconazole+SX, azoxystrobin+SX, basic copper sulfate+SX, benalaxyl+SX, benalaxyl-M+SX, benodanil+SX, benomyl+SX, benthiavalicarb+SX, benthiavalicarb-isopropyl+SX, benzovindiflupyr+SX, binapacryl+SX, biphenyl+SX, bitertanol+SX, bixafen+SX, blasticidin-S+SX, Bordeaux mixture+SX, boscalid+SX, bromothalonil+SX, bromuconazole+SX, bupirimate+SX, captafol+SX, captan+SX, carbendazim+SX, carboxin+SX, carpropamid+SX, chinomethionat+SX, chitin+SX, chloroneb+SX, chlorothalonil+SX, chlozolinate+SX, colletochlorin B+SX, copper(II) acetate+SX, copper(II) hydroxide+SX, copper oxychloride+SX, copper(II) sulfate+SX, coumoxystrobin+SX, cyazofamid+SX, cyflufenamid+SX, cymoxanil+SX, cyproconazole+SX, cyprodinil+SX, dichlobentiazox+SX, dichlofluanid+SX, diclocymet+SX, diclomezine+SX, dicloran+SX, diethofencarb+SX, difenoconazole+SX, diflumetorim+SX, dimethachlone+SX, dimethirimol+SX, dimethomorph+SX, dimoxystrobin+SX, diniconazole+SX, diniconazole-M+SX, dinocap+SX, dipotassium hydrogenphosphite+SX, dipymetitrone+SX, dithianon+SX, dodecylbenzenesulphonic acid bisethylenediamine copper(II) salt+SX, dodemorph+SX, dodine+SX, edifenphos+SX, enoxastrobin+SX, epoxiconazole+SX, etaconazole+SX, ethaboxam+SX, ethirimol+SX, etridiazole+SX, extract from Melaleuca alternifolia+SX, extract from Reynoutria sachalinensis+SX, extract from the cotyledons of lupine plantlets (“BLAD”)+SX, extract of Allium sativum+SX, extract of Equisetum arvense+SX, extract of Tropaeolum majus+SX, famoxadone+SX, fenamidone+SX, fenaminstrobin+SX, fenarimol+SX, fenbuconazole+SX, fenfuram+SX, fenhexamid+SX, fenoxanil+SX, fenpiclonil+SX, fenpicoxamid+SX, fenpropidin+SX, fenpropimorph+SX, fenpyrazamine+SX, fentin acetate+SX, fentin chloride+SX, fentin hydroxide+SX, ferbam+SX, ferimzone+SX, florylpicoxamid+SX, fluazinam+SX, flubeneteram+SX, fludioxonil+SX, flufenoxystrobin+SX, fluindapyr+SX, flumorph+SX, fluopicolide+SX, fluopyram+SX, fluopimomide+SX, fluoroimide+SX, fluoxapiprolin+SX, fluoxastrobin+SX, fluquinconazole+SX, flusilazole+SX, flusulfamide+SX, flutianil+SX, flutolanil+SX, flutriafol+SX, fluxapyroxad+SX, folpet+SX, fosetyl+SX, fosetyl-aluminium+SX, fuberidazole+SX, furalaxyl+SX, furametpyr+SX, guazatine+SX, hexaconazole+SX, hymexazole+SX, imazalil+SX, imibenconazole+SX, iminoctadine+SX, iminoctadine triacetate+SX, inpyrfluxam+SX, iodocarb+SX, ipconazole+SX, ipfentrifluconazole+SX, ipflufenoquin+SX, iprobenfos+SX, iprodione+SX, iprovalicarb+SX, isofetamid+SX, isoflucypram+SX, isoprothiolane+SX, isopyrazam+SX, isotianil+SX, kasugamycin+SX, kresoxim-methyl+SX, laminarin+SX, leaves and bark of Quercus+SX, mancozeb+SX, mandestrobin+SX, mandipropamid+SX, maneb+SX, mefentrifluconazole+SX, mepanipyrim+SX, mepronil+SX, meptyldinocap+SX, metalaxyl+SX, metalaxyl-M+SX, metconazole+SX, methasulfocarb+SX, metiram+SX, metominostrobin+SX, metrafenone+SX, metyltetraprole+SX, mineral oils+SX, myclobutanil+SX, naftifine+SX, nuarimol+SX, octhilinone+SX, ofurace+SX, orysastrobin+SX, oxadixyl+SX, oxathiapiprolin+SX, oxine-copper+SX, oxolinic acid+SX, oxpoconazole+SX, oxpoconazole fumarate+SX, oxycarboxin+SX, oxytetracycline+SX, pefurazoate+SX, penconazole+SX, pencycuron+SX, penflufen+SX, penthiopyrad+SX, phenamacril+SX, phosphorous acid+SX, phthalide+SX, picarbutrazox+SX, picoxystrobin+SX, piperalin+SX, polyoxins+SX, potassium hydrogencarbonate+SX, potassium dihydrogenphosphite+SX, probenazole+SX, prochloraz+SX, procymidone+SX, propamidine+SX, propamocarb+SX, propiconazole+SX, propineb+SX, proquinazid+SX, prothiocarb+SX, prothioconazole+SX, pydiflumetofen+SX, pyraclostrobin+SX, pyrametostrobin+SX, pyraoxystrobin+SX, pyrapropoyne+SX, pyraziflumid+SX, pyrazophos+SX, pyribencarb+SX, pyributicarb+SX, pyridachlometyl+SX, pyrifenox+SX, pyrimethanil+SX, pyrimorph+SX, pyriofenone+SX, pyrisoxazole+SX, pyroquilon+SX, Quillaja extract+SX, quinconazole+SX, quinofumelin+SX, quinoxyfen+SX, quintozene+SX, Saponins of Chenopodium quinoa+SX, sedaxane+SX, silthiofam+SX, simeconazole+SX, sodium hydrogencarbonate+SX, spiroxamine+SX, streptomycin+SX, sulfur+SX, tebuconazole+SX, tebufloquin+SX, teclofthalam+SX, tecnazene+SX, terbinafine+SX, tetraconazole+SX, thiabendazole+SX, thifluzamide+SX, thiophanate+SX, thiophanate-methyl+SX, thiram+SX, thymol+SX, tiadinil+SX, tolclofos-methyl+SX, tolfenpyrad+SX, tolprocarb+SX, tolylfluanid+SX, triadimefon+SX, triadimenol+SX, triazoxide+SX, triclopyricarb+SX, tricyclazole+SX, tridemorph+SX, trifloxystrobin+SX, triflumizole+SX, triforine+SX, triticonazole+SX, validamycin+SX, valifenalate+SX, vinclozolin+SX, yellow mustard powder+SX, zinc thiazole+SX, zineb+SX, ziram+SX, zoxamide+SX, N′-[4-({3-[(4-chlorophenyl)methyl]-1,2,4-thiadiazol-5-yl}oxy)-2,5-dimethylphenyl]-N-ethyl-N-methylmethanimidamide (1202781-91-6)+SX, N′-{4-[(4,5-dichlorothiazol-2-yl)oxy]-2,5-dimethylphenyl}-N-ethyl-N-methylmethanimidamide (929908-57-6)+SX, N′-(2,5-dimethyl-4-phenoxyphenyl)-N-ethyl-N-methylmethanimidamide (1052688-31-9)+SX, N′-[5-choro-4-(2-fluorophenoxy)-2-methylphenyl)-N-ethyl-N-methylmethanimidamide (2055589-28-9)+SX, N′-[2-choro-4-(2-fluorophenoxy)-5-methylphenyl)-N-ethyl-N-methylmethanimidamide (2055756-21-1)+SX, N′-[4-(1-hydroxy-1-phenyl-2,2,2-trifluoroethyl)-2-methyl-5-methoxyphenyl)-N-isopropyl-N-methylmethanimidamide (2101814-55-3)+SX, N′-[5-bromo-6-(1-methyl-2-propoxyethoxy)-2-methylpyridin-3-yl)-N-ethyl-N-methylmethanimidamide (1817828-69-5)+SX, 4-(2-bromo-4-fluorophenyl)-N-(2-chloro-6-fluorophenyl)-1,3-dimethyl-1H-pyrazol-5-amine (1362477-26-6)+SX, 2-[6-(3-fluoro-4-methoxyphenyl)-5-methylpyridin-2-yl]quinazoline (1257056-97-5)+SX, 5-fluoro-4-imino-3-methyl-1-tosyl-3,4-dihydropyrimidin-2(1H)-one (1616664-98-2)+SX, ethyl (2Z)-3-amino-2-cyano-3-phenylacrylate (39491-78-6)+SX, N-[(2-chlorothiazol-5-yl)methyl]-N-ethyl-6-methoxy-3-nitropyridin-2-amine (1446247-98-8)+SX, 5-(4-chlorobenzyl)-2-(chloromethyl)-2-methyl-1-(1H-1,2,4-triazol-1-ylmethyl)cyclopentan-1-ol (1394057-11-4)+SX, (1R, 2S, 5S)-5-(4-chlorobenzyl)-2-(chloromethyl)-2-methyl-1-(1H-1,2,4-triazol-1-ylmethyl)cyclopentan-1-ol (1801930-06-2)+SX, (1S, 2R, 5R)-5-(4-chlorobenzyl)-2-(chloromethyl)-2-methyl-1-(1H-1,2,4-triazol-1-ylmethyl)cyclopentan-1-ol (1801930-07-3)+SX, 2-(chloromethyl)-5-(4-fluorobenzyl)-2-methyl-1-(1H-1,2,4-triazol-1-ylmethyl)cyclopentan-1-ol (1394057-13-6)+SX, (1R, 2S, 5S)-2-(chloromethyl)-5-(4-fluorobenzyl)-2-methyl-1-(1H-1,2,4-triazol-1-ylmethyl)cyclopentan-1-ol (1801930-08-4)+SX, (1S, 2R, 5R)-2-(chloromethyl)-5-(4-fluorobenzyl)-2-methyl-1-(1H-1,2,4-triazol-1-ylmethyl)cyclopentan-1-ol (1801930-09-5)+SX, methyl 3-[(4-chlorophenyl)methyl]-2-hydroxy-1-methyl-2-(1H-1,2,4-triazol-1-ylmethyl)cyclopentan-1-carboxylate (1791398-02-1)+SX, 1-(2,4-difluorophenyl)-2-(1H-1,2,4-triazol-1-yl)-1-[1-(4-bromo-2,6-difluorophenoxy)cyclopropyl]ethanol (2019215-86-0)+SX, 1-(2,4-difluorophenyl)-2-(1H-1,2,4-triazol-1-yl)-1-[1-(4-chloro-2,6-difluorophenoxy)cyclopropyl]ethanol (2019215-84-8)+SX, 1-[2-(1-chlorocyclopropyl)-3-(2-fluorophenyl)-2-hydroxypropyl]-1H-imidazole-5-carbonitrile (2018316-13-5)+SX, 1-[2-(1-chlorocyclopropyl)-3-(2,3-difluorophenyl)-2-hydroxypropyl]-1H-imidazole-5-carbonitrile (2018317-25-2)+SX, 4-({6-[2-(2,4-difluorophenyl)-1,1-difluoro-2-hydroxy-3-(1H-1,2,4-triazol-1-yl)propyl]pyridin-3-yl}oxy)benzonitrile (2046300-61-0)+SX, 2-[6-(4-bromophenoxy)-2-(trifluoromethyl)pyridin-3-yl]-1-(1H-1,2,4-triazol-1-yl)propan-2-ol (2082661-43-4)+SX, 2-[6-(4-chlorophenoxy)-2-(trifluoromethyl)pyridin-3-yl]-1-(1H-1,2,4-triazol-1-yl)propan-2-ol (2082660-27-1)+SX, methyl ({2-methyl-5-[1-(4-methoxy-2-methylphenyl)-1H-pyrazol-3-yl]phenyl}methyl)carbamate (1605879-98-8)+SX, 2-(difluoromethyl)-N-[1,1,3-trimethyl-2,3-dihydro-1H-inden-4-yl]pyridine-3-carboxamide (1616239-21-4)+SX, 2-(difluoromethyl)-N-[3-ethyl-1,1-dimethyl-2,3-dihydro-1H-inden-4-yl]pyridine-3-carboxamide (1847460-02-9)+SX, 2-(difluoromethyl)-N-[3-propyl-1,1-dimethyl-2,3-dihydro-1H-inden-4-yl]pyridine-3-carboxamide (1847460-05-2)+SX, (2E,3Z)-5-{[1-(4-chlorophenyl)-1H-pyrazol-3-yl]oxy}-2-(methoxyimino)-N,3-dimethylpent-3-enamide (1445331-27-0)+SX, (2E,3Z)-5-{[1-(2,4-dichlorophenyl)-1H-pyrazol-3-yl]oxy}-2-(methoxyimino)-N,3-dimethylpent-3-enamide (1445331-54-3)+SX, 5-chloro-4-({2-[6-(4-chlorophenoxy)pyridin-3-yl]ethyl}amino)-6-methylpyrimidine (1605340-92-8)+SX, N-(1-benzyl-1,3-dimethylbutyl)-8-fluoroquinoline-3-carboxamide (2132414-04-9)+SX, N-(1-benzyl-3,3,3-trifluoro-1-methylpropyl)-8-fluoroquinoline-3-carboxamide (2132414-00-5)+SX, 4,4-dimethyl-2-({4-[5-(trifluoromethyl)-1,2,4-oxadiazol-3-yl]phenyl}methyl)isoxazolidin-3-one (2098918-25-1)+SX, 5,5-dimethyl-2-({4-[5-(trifluoromethyl)-1,2,4-oxadiazol-3-yl]phenyl}methyl)isoxazolidin-3-one (2098918-26-2)+SX, N-acetyl-2-(ethanesulfonyl)-N-[2-(methoxycarbonyl)-4-(trifluoromethoxy) phenyl]-4-(trifluoromethyl) benzamide (2043675-28-9)+SX, Agrobacterium radiobactor strain K1026+SX, Agrobacterium radiobactor strain K84+SX, Bacillus amyloliquefaciens (Aveo (trademark) EZ Nematicide)+SX, Bacillus amyloliquefaciens strain AT332+SX, Bacillus amyloliquefaciens strain B3+SX, Bacillus amyloliquefaciens strain D747+SX, Bacillus amyloliquefaciens strain DB101+SX, Bacillus amyloliquefaciens strain DB102+SX, Bacillus amyloliquefaciens strain GB03+SX, Bacillus amyloliquefaciens strain FZB24+SX, Bacillus amyloliquefaciens strain FZB42+SX, Bacillus amyloliquefaciens strain IN937a+SX, Bacillus amyloliquefaciens strain MBI600+SX, Bacillus amyloliquefaciens strain QST713+SX, Bacillus amyloliquefaciens isolate strain B246+SX, Bacillus amyloliquefaciens strain F727+SX, Bacillus amyloliquefaciens subsp. plantarum strain D747+SX, Bacillus licheniformis strain HB-2+SX, Bacillus licheniformis strain SB3086+SX, Bacillus pumilus strain AQ717+SX, Bacillus pumilus strain BUF-33+SX, Bacillus pumilus strain GB34+SX, Bacillus pumilus strain QST2808+SX, Bacillus simplex strain CGF2856+SX, Bacillus subtilis strain AQ153+SX, Bacillus subtilis strain AQ743+SX, Bacillus subtilis strain BU1814+SX, Bacillus subtilis strain D747+SX, Bacillus subtilis strain DB101+SX, Bacillus subtilis strain FZB24+SX, Bacillus subtilis strain GB03+SX, Bacillus subtilis strain HAI0404+SX, Bacillus subtilis strain IAB/BS03+SX, Bacillus subtilis strain MBI600+SX, Bacillus subtilis strain QST30002/AQ30002+SX, Bacillus subtilis strain QST30004/AQ30004+SX, Bacillus subtilis strain QST713+SX, Bacillus subtilis strain QST714+SX, Bacillus subtilis var. Amyloliquefaciens strain FZB24+SX, Bacillus subtilis strain Y1336+SX, Burkholderia cepacia+SX, Burkholderia cepacia type Wisconsin strain J82+SX, Burkholderia cepacia type Wisconsin strain M54+SX, Candida oleophila strain O+SX, Candida saitoana+SX, Chaetomium cupreum+SX, Clonostachys rosea+SX, Coniothyrium minitans strain CGMCC8325+SX, Coniothyrium minitans strain CON/M/91-8+SX, Cryptococcus albidus+SX, Erwinia carotovora subsp. carotovora strain CGE234M403+SX, Fusarium oxysporum strain Fo47+SX, Gliocladium catenulatum strain J1446+SX, Paenibacillus polymyxa strain AC-1+SX, Paenibacillus polymyxa strain BS-0105+SX, Pantoea agglomerans strain E325+SX, Phlebiopsis gigantea strain VRA1992+SX, Pseudomonas aureofaciens strain TX-1+SX, Pseudomonas chlororaphis strain 63-28+SX, Pseudomonas chlororaphis strain AFS009+SX, Pseudomonas chlororaphis strain MA342+SX, Pseudomonas fluorescens strain 1629RS+SX, Pseudomonas fluorescens strain A506+SX, Pseudomonas fluorescens strain CL145A+SX, Pseudomonas fluorescens strain G7090+SX, Pseudomonas sp. strain CAB-02+SX, Pseudomonas syringae strain 742RS+SX, Pseudomonas syringae strain MA-4+SX, Pseudozyma flocculosa strain PF-A22UL+SX, Pseudomonas rhodesiae strain HAI-0804+SX, Pythium oligandrum strain DV74+SX, Pythium oligandrum strain M1+SX, Streptomyces griseoviridis strain K61+SX, Streptomyces lydicus strain WYCD108US+SX, Streptomyces lydicus strain WYEC108+SX, Talaromyces flavus strain SAY-Y-94-01+SX, Talaromyces flavus strain V117b+SX, Trichoderma asperellum strain ICC012+SX, Trichoderma asperellum SKT-1+SX, Trichoderma asperellum strain T25+SX, Trichoderma asperellum strain T34+SX, Trichoderma asperellum strain TV1+SX, Trichoderma atroviride strain CNCM 1-1237+SX, Trichoderma atroviride strain LC52+SX, Trichoderma atroviride strain IMI 206040+SX, Trichoderma atroviride strain SC1+SX, Trichoderma atroviride strain SKT-1+SX, Trichoderma atroviride strain T11+SX, Trichoderma gamsii strain ICC080+SX, Trichoderma harzianum strain 21+SX, Trichoderma harzianum strain DB104+SX, Trichoderma harzianum strain DSM 14944+SX, Trichoderma harzianum strain ESALQ-1303+SX, Trichoderma harzianum strain ESALQ-1306+SX, Trichoderma harzianum strain IIHR-Th-2+SX, Trichoderma harzianum strain ITEM908+SX, Trichoderma harzianum strain kd+SX, Trichoderma harzianum strain MO1+SX, Trichoderma harzianum strain SF+SX, Trichoderma harzianum strain T22+SX, Trichoderma harzianum strain T39+SX, Trichoderma harzianum strain T78+SX, Trichoderma harzianum strain TH35+SX, Trichoderma polysporum strain IMI206039+SX, trichoderma stromaticum+SX, Trichoderma virens strain G-41+SX, Trichoderma virens strain GL-21+SX, Trichoderma viride+SX, Variovorax paradoxus strain CGF4526+SX, Harpin protein+SX, N-acetyl-2-(ethanesulfonyl)-N-[2-(methoxycarbonyl)-4-(trifluoromethoxy)phenyl]-4-(trifluoromethyl)benzamide (2043675-28-9)+SX, (2S,3S)-3-(2-methylphenyl)butan-2-yl N-[(3-acetoxy-4-methoxypyridin-2-yl)carbonyl]-L-alaninate (2376210-00-1)+SX, (2S,3S)-3-(4-fluoro-2-methylphenyl)butan-2-yl N-[(3-acetoxy-4-methoxypyridin-2-yl)carbonyl]-L-alaninate+SX, (2S,3S)-3-(4-methoxy-2-methylphenyl)butan-2-yl N-[(3-acetoxy-4-methoxypyridin-2-yl)carbonyl]-L-alaninate+SX, (2S,3S)-3-(2,4-dimethylphenyl)butan-2-yl N-[(3-acetoxy-4-methoxypyridin-2-yl)carbonyl]-L-alaninate (2376209-13-9)+SX, (2S,3S)-3-(2-methylphenyl)butan-2-yl N-({3-[(2-methylpropanoyl)oxy]-4-methoxypyridin-2-yl}carbonyl)-L-alaninate (2376210-02-3)+SX, (2S,3S)-3-(4-fluoro-2-methylphenyl)butan-2-yl N-({3-[(2-methylpropanoyl)oxy]-4-methoxypyridin-2-yl}carbonyl)-L-alaninate+SX, (2S,3S)-3-(4-methoxy-2-methylphenyl)butan-2-yl N-({3-[(2-methylpropanoyl)oxy]-4-methoxypyridin-2-yl}carbonyl)-L-alaninate (2376209-40-2)+SX, (2S,3S)-3-(2,4-dimethylphenyl)butan-2-yl N-({3-[(2-methylpropanoyl)oxy]-4-methoxypyridin-2-yl}carbonyl)-L-alaninate (2376209-15-1)+SX, N′-(2-choro-4-phenoxy-5-methylphenyl)-N-ethyl-N-methylmethanimidamide (2062599-39-5)+SX, (2S,3S)-3-(2-methylphenyl)butan-2-yl N-[(3-hydroxy-4-methoxypyridin-2-yl)carbonyl]-L-alaninate+SX, (2S,3S)-3-(4-fluoro-2-methylphenyl)butan-2-yl N-[(3-hydroxy-4-methoxypyridin-2-yl)carbonyl]-L-alaninate+SX, (2S,3S)-3-(4-methoxy-2-methylphenyl)butan-2-yl N-[(3-hydroxy-4-methoxypyridin-2-yl)carbonyl]-L-alaninate+SX, (2S,3S)-3-(2,4-dimethylphenyl)butan-2-yl N-[(3-hydroxy-4-methoxypyridin-2-yl)carbonyl]-L-alaninate+SX, (2S,3S)-3-(4-fluoro-2-methylphenyl)-4-methylpentan-2-yl N-[(3-acetoxy-4-methoxypyridin-2-yl)carbonyl]-L-alaninate+SX, (2S,3S)-3-(2-fluoro-4-methylphenyl)-4-methylpentan-2-yl N-[(3-acetoxy-4-methoxypyridin-2-yl)carbonyl]-L-alaninate+SX, (2S,3S)-3-(2,4-difluorophenyl)-4-methylpentan-2-yl N-[(3-acetoxy-4-methoxypyridin-2-yl)carbonyl]-L-alaninate+SX, (2S,3S)-3-(2,4-dimethylphenyl)-4-methylpentan-2-yl N-[(3-acetoxy-4-methoxypyridin-2-yl)carbonyl]-L-alaninate+SX, (2S,3S)-3-(4-fluoro-2-methoxylphenyl)-4-methylpentan-2-yl N-[(3-acetoxy-4-methoxypyridin-2-yl)carbonyl]-L-alaninate+SX, (2S,3S)-3-(4-fluoro-2-methylphenyl)-4-methylpentan-2-yl N-{[3-(acetoxymethoxy)-4-methoxypyridin-2-yl]carbonyl}-L-alaninate+SX, (2S,3S)-3-(2-fluoro-4-methylphenyl)-4-methylpentan-2-yl N-{[3-(acetoxymethoxy)-4-methoxypyridin-2-yl]carbonyl}-L-alaninate+SX, (2S,3S)-3-(2,4-difluorophenyl)-4-methylpentan-2-yl N-{[3-(acetoxymethoxy)-4-methoxypyridin-2-yl]carbonyl}-L-alaninate+SX, (2S,3S)-3-(2,4-dimethylphenyl)-4-methylpentan-2-yl N-{[3-(acetoxymethoxy)-4-methoxypyridin-2-yl]carbonyl}-L-alaninate+SX, (2S,3S)-3-(4-fluoro-2-methoxylphenyl)-4-methylpentan-2-yl N-{[3-(acetoxymethoxy)-4-methoxypyridin-2-yl]carbonyl}-L-alaninate+SX, (2S,3S)-3-(4-fluoro-2-methylphenyl)-4-methylpentan-2-yl N-({3-[(isobutyryloxy)methoxy]-4-methoxypyridin-2-yl}carbonyl)-L-alaninate+SX, (2S,3S)-3-(2-fluoro-4-methylphenyl)-4-methylpentan-2-yl N-({3-[(isobutyryloxy)methoxy]-4-methoxypyridin-2-yl}carbonyl)-L-alaninate+SX, (2S,3S)-3-(2,4-difluorophenyl)-4-methylpentan-2-yl N-({3-[(isobutyryloxy)methoxy]-4-methoxypyridin-2-yl}carbonyl)-L-alaninate+SX, (2S,3S)-3-(2,4-dimethylphenyl)-4-methylpentan-2-yl N-({3-[(isobutyryloxy)methoxy]-4-methoxypyridin-2-yl}carbonyl)-L-alaninate+SX, (2S,3S)-3-(4-fluoro-2-methoxylphenyl)-4-methylpentan-2-yl N-({3-[(isobutyryloxy)methoxy]-4-methoxypyridin-2-yl}carbonyl)-L-alaninate+SX, (2S,3S)-3-(4-fluoro-2-methylphenyl)-4-methylpentan-2-yl N-[(3-hydroxy-4-methoxypyridin-2-yl)carbonyl]-L-alaninate+SX, (2S,3S)-3-(2-fluoro-4-methylphenyl)-4-methylpentan-2-yl N-[(3-hydroxy-4-methoxypyridin-2-yl)carbonyl]-L-alaninate+SX, (2S,3S)-3-(2,4-difluorophenyl)-4-methylpentan-2-yl N-[(3-hydroxy-4-methoxypyridin-2-yl)carbonyl]-L-alaninate+SX, (2S,3S)-3-(2,4-dimethylphenyl)-4-methylpentan-2-yl N-[(3-hydroxy-4-methoxypyridin-2-yl)carbonyl]-L-alaninate+SX, (2S,3S)-3-(4-fluoro-2-methoxylphenyl)-4-methylpentan-2-yl N-[(3-hydroxy-4-methoxypyridin-2-yl)carbonyl]-L-alaninate+SX, 3-(4-bromo-7-fluoroindol-1-yl)butan-2-yl N-[(3-hydroxy-4-methoxypyridin-2-yl)carbonyl]-L-alaninate+SX, 3-(7-bromoindol-1-yl)butan-2-yl N-[(3-hydroxy-4-methoxypyridin-2-yl)carbonyl]-L-alaninate+SX, 3-(7-bromo-4-fluoroindol-1-yl)butan-2-yl N-[(3-hydroxy-4-methoxypyridin-2-yl)carbonyl]-L-alaninate+SX, 3-(3,5-dichloropyridin-2-yl)butan-2-yl N-[(3-hydroxy-4-methoxypyridin-2-yl)carbonyl]-L-alaninate+SX, 3-(3,5-dichloropyridin-2-yl)butan-2-yl N-{[3-(acetoxymethoxy)-4-methoxypyridin-2-yl]carbonyl}-L-alaninate+SX, (1S)-1-[1-(naphthalen-1-yl)cyclopropyl]ethyl N-[(3-hydroxy-4-methoxypyridin-2-yl)carbonyl]-L-alaninate+SX, (1S)-1-[1-(naphthalen-1-yl)cyclopropyl]ethyl N-[(3-acetoxy-4-methoxypyridin-2-yl)carbonyl]-L-alaninate+SX, (1S)-1-[1-(naphthalen-1-yl)cyclopropyl]ethyl N-{[3-(acetoxymethoxy)-4-methoxypyridin-2-yl]carbonyl}-L-alaninate+SX, chloroinconazide+SX, N-ethyl-2-methyl-N-({4-[5-(trifluoromethyl)-1,2,4-oxadiazol-3-yl]phenyl}methyl)propanamide+SX, N,2-dimethoxy-N-({4-[5-(trifluoromethyl)-1,2,4-oxadiazol-3-yl]phenyl}methyl)propanamide+SX, N-methoxy-N-({4-[5-(trifluoromethyl)-1,2,4-oxadiazol-3-yl]phenyl}methyl)cyclopropanecarboxamide+SX, N-methoxy-N′-methyl-N-({4-[5-(trifluoromethyl)-1,2,4-oxadiazol-3-yl]phenyl}methyl)urea+SX, N′-ethyl-N-methoxy-N-({4-[5-(trifluoromethyl)-1,2,4-oxadiazol-3-yl]phenyl}methyl)urea+SX, N,N′-dimethoxy-N-({4-[5-(trifluoromethyl)-1,2,4-oxadiazol-3-yl]phenyl}methyl)urea+SX, N-(2-fluorophenyl)-4-[5-(trifluoromethyl)-1,2,4-oxadiazol-3-yl]benzamide+SX, N-[2-(difluoromethoxy)phenyl]-4-[5-(trifluoromethyl)-1,2,4-oxadiazol-3-yl]benzamide+SX, N-[2-(difluoromethoxy)-4-fluorophenyl]-4-[5-(trifluoromethyl)-1,2,4-oxadiazol-3-yl]benzamide+SX.

Combinations of the present ingredient of the above Group (c) and the present compound C:
1-methylcyclopropene+SX, 1,3-diphenylurea+SX, 2,3,5-triiodobenzoic acid+SX, IAA ((1H-indol-3-yl) acetic acid)+SX, IBA (4-(1H-indol-3-yl)butyric acid)+SX, MCPA (2-(4-chloro-2-methylphenoxy)acetic acid)+SX, MCPB (4-(4-chloro-2-methylphenoxy)butyric acid)+SX, 4-CPA (4-chlorophenoxyacetic acid)+SX, 5-aminolevulinic acid hydrochloride+SX, 6-benzylaminopurine+SX, abscisic acid+SX, AVG (aminoethoxyvinylglycine)+SX, ancymidol+SX, butralin+SX, calcium carbonate+SX, calcium chloride+SX, calcium formate+SX, calcium peroxide+SX, calcium polysulfide+SX, calcium sulfate+SX, chlormequat-chloride+SX, chlorpropham+SX, choline chloride+SX, cloprop+SX, cyanamide+SX, cyclanilide+SX, daminozide+SX, decan-1-ol+SX, dichlorprop+SX, dikegulac+SX, dimethipin+SX, diquat+SX, ethephon+SX, ethychlozate+SX, flumetralin+SX, flurprimidol+SX, forchlorfenuron+SX, formononetin+SX, Gibberellin A+SX, Gibberellin A3+SX, inabenfide+SX, Kinetin+SX, lipochitooligosaccharide SP104+SX, maleic hydrazide+SX, mefluidide+SX, mepiquat-chloride+SX, oxidized glutathione+SX, pacrobutrazol+SX, pendimethalin+SX, prohexandione-calcium+SX, prohydrojasmon+SX, pyraflufen-ethyl+SX, sintofen+SX, sodium 1-naphthaleneacetate+SX, sodium cyanate+SX, streptmycin+SX, thidiazuron+SX, triapenthenol+SX, Tribufos+SX, trinexapac-ethyl+SX, uniconazole-P+SX, 2-(naphthalen-1-yl)acetamide+SX, [4-oxo-4-(2-phenylethyl)amino]butyric acid+SX, methyl 5-(trifluoromethyl)-benzo[b]thiophen-2-carboxylate+SX, 3-[(6-chloro-4-phenylquinazolin-2-yl)amino]propan-1-ol+SX, Claroideoglomus etunicatum+SX, Claroideoglomus claroideum+SX, Funneliformis mosseae+SX, Gigaspora margarita+SX, Gigaspora rosea+SX, Glomus aggregatum+SX, Glomus deserticola+SX, Glomus monosporum+SX, Paraglomus brasillianum+SX, Rhizophagus clarus+SX, Rhizophagus intraradices RTI-801+SX, Rhizophagus irregularis DAOM 197198+SX, Azorhizobium caulinodans+SX, Azospirillum amazonense+SX, Azospirillum brasilense XOH+SX, Azospirillum brasilense Ab-V5+SX, Azospirillum brasilense Ab-V6+SX, Azospirillum caulinodans+SX, Azospirillum halopraeferens+SX, Azospirillum irakense+SX, Azospirillum lipoferum+SX, Bradyrhizobium elkanii SEMIA 587+SX, Bradyrhizobium elkanii SEMIA 5019+SX, Bradyrhizobium japonicum TA-11+SX, Bradyrhizobium japonicum USDA 110+SX, Bradyrhizobium liaoningense+SX, Bradyrhizobium lupini+SX, Delftia acidovorans RAY209+SX, Mesorhizobium ciceri+SX, Mesorhizobium huakii+SX, Mesorhizobium loti+SX, Rhizobium etli+SX, Rhizobium galegae+SX, Rhizobium leguminosarum bv. Phaseoli+SX, Rhizobium leguminosarum bv. Trifolii+SX, Rhizobium leguminosarum bv. Viciae+SX, Rhizobium trifolii+SX, Rhizobium tropici+SX, Sinorhizobium fredii+SX, Sinorhizobium meliloti+SX, Zucchini Yellow Mosaik Virus weak strain+SX.

Combinations of the present ingredient of the above Group (d) and the present compound C:
anthraquinone+SX, deet+SX, icaridin+SX.

The ratio of the present compound C to the Present ingredient includes, but not limited thereto, as a ratio by weight (the compound of the present invention:the Present ingredient) 1,000:1 to 1:1,000, 500:1 to 1:500, 100:1 to 1:100, 50:1, 20:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10. 1:20, and 1:50, and the others.

The present compound, the present compound C, the present compound D, or the composition A is usually mixed with solid carrier(s), liquid carrier(s), oil(s), and/or surfactant(s), and if necessary, added by the other auxiliary agents for formulation, to formulate into emulsifiable concentrates, oil solutions, dust formulations, granules, wettable powders, wettable dispersible granules, flowables, dry flowables, microcapsules and the others. In these formulations, the present compound, the present compound C, the present compound D, or the composition A is contained in usually 0.1 to 99% by weight, preferably 0.2 to 90%.

Examples of the solid carrier include fine powders or granules of clays (for example, kaolin clay, diatomaceous earth, bentonite, or acid white clay), dry silica, wet silica, hydrated silica, talcs, ceramics, other inorganic minerals (for example, sericite, quartz, sulfur, active carbon, or calcium carbonate); as well as synthetic resins (for example, polyester resins such as polypropylene, polyacrylonitrile, polymethyl methacrylate or polyethylene terephthalate; nylon resins (for example, nylon-6, nylon-11, or nylon-66); polyamide resins; polyvinyl chloride, polyvinylidene chloride, vinyl chloride-propylene copolymers, and the others).

Examples of the liquid carriers include water; alcohols (for example, methanol, ethanol, isopropyl alcohol, butanol, hexanol, benzyl alcohol, ethylene glycol, propylene glycol, or phenoxy ethanol); ketones (for example, acetone, methyl ethyl ketone, or cyclohexanone); aromatic hydrocarbons (for example, toluene, xylene, ethyl benzene, dodecyl benzene, phenyl xylyl ethane, or methylnaphthalene); aliphatic hydrocarbons (for example, hexane, cyclohexane, kerosene, or light oil); esters (for example, ethyl acetate, butyl acetate, isopropyl myristate, ethyl oleate, diisopropyl adipate, diisobutyl adipate, or propylene glycol monomethyl ether acetate); nitriles (for example, acetonitrile, or isobutyronitrile); ethers (for example, diisopropyl ether, 1,4-dioxane, 1,2-dimethoxyethane, diethyleneglycol dimethyl ether, diethylene glycol monomethyl ether, propylene glycol monomethyl ether, dipropylene glycol monomethyl ether, or 3-methoxy-3-methyl-1-butanol); amides (for example, DMF, or N,N-dimethylacetamide); sulfoxides (for example, dimethyl sulfoxide); propylene carbonate; and vegetable oils (for example, soybean oil or cottonseed oil).

Examples of the surfactants include nonionic surfactants such as polyoxyethylenated alkyl ethers, polyoxyethylenated alkyl aryl ethers, and polyethylene glycol fatty acid esters; and anionic surfactants such as alkyl sulfonates, alkylbenzene sulfonates and alkyl sulfates. Specific examples thereof include Nimbus (registered trademark), Assist (registered trademark), Aureo (registered trademark), Iharol (registered trademark), Silwet L-77 (registered trademark), BreakThru (registered trademark), SundanceII (registered trademark), Induce (registered trademark), Penetrator (registered trademark), AgriDex (registered trademark), Lutensol A8 (registered trademark), NP-7 (registered trademark), Triton (registered trademark), Nufilm (registered trademark), Emulgator NP7 (registered trademark), Emulad (registered trademark), TRITON X 45 (registered trademark), AGRAL 90 (registered trademark), AGROTIN (registered trademark), ARPON (registered trademark), EnSpray N (registered trademark), and BANOLE (registered trademark), and the others.

Examples of the other auxiliary agents for formulation include a binder, a dispersant, a colorant and a stabilizer. Specific examples include casein, gelatin, polysaccharides (for example, starch, gum arabic, cellulose derivatives and alginic acid), lignin derivatives, bentonite, water-soluble synthetic polymers (for example, polyvinyl alcohol, polyvinyl pyrrolidone and polyacrylic acids), acidic isopropyl phosphate, 2,6-di-tert-butyl-4-methylphenol, and a mixture of 2-tert-butyl-4-methoxyphenol and 3-tert-butyl-4-methoxyphenol.

Examples of an application of the present compound, the present compound C, the present compound D or the composition A include a spreading to stems and leaves of soybeans, an application to seeds, and an application to soil for cultivating soybeans.

The application dose of the present compound, the present compound C, or the present compound D may be varied depending on a climate condition, a formulation form, an application period, an application method, an application site, plant diseases to be controlled, plant to be applied, and the others. In the cases where these compounds are spread to stems and leaves of soybean or are applied to soil for cultivating soybeans, the application dose thereof is within a range of usually 1 to 500 g, preferably 2 to 200 g per 1,000 m². In the cases where these compounds are applied to seeds, the application dose thereof is within a range of 0.01 to 100 g, preferably 0.01 to 50 g per 1 Kg of seeds. The application dose of the composition A is within a range of usually 1 to 500 g per 1,000 m² in the case where it is spread to stems and leaves of soybean or are applied to soil for cultivating soybeans. In the cases where it is applied to seeds, the application dose thereof is within a range of usually 0.001 to 100 g per 1 Kg of seeds. The emulsifiable concentrate, the wettable powder, the suspension etc., is usually applied by diluting them with water. In these cases, the concentration of the present compound, the present compound C, the present compound D, or the composition A after the dilution is within a range of usually 0.0005 to 2% by weight, preferably 0.005 to 2% by weight. The dust formulation or the granular formulation, etc., is usually applied as itself without diluting them.

Seeds are treated with the present compound C or the composition A to obtain seeds carrying the present compound C or the composition A. The seeds carrying the present compound C or the composition A means seeds in the state where the present compound C or the composition A is adhered to a surface of the seeds. Also, the present compound C or the composition A which are may be adhered on the surface of the seeds may be permeated from the surface to the interior of the plant.

Also, when the composition A is adhered on the surface of the seeds, a layer consisting of single active ingredient may be multiply overlapped, a plural of the active ingredients may be mixed to form a single layer, a layer consisting of the single active ingredient and a layer consisting of the plural of the active ingredients may be multiply overlapped, or a layer consisting of the plural of the active ingredients may be multiply overlapped. In the seeds used for the seed treatment, any materials other than the present compound C or the composition A may be adhered before or after being treated with the present compound C or the composition A.

Examples of the application to seeds (or seed treatments) include an application of the present compound C or the composition A to seeds, and specific examples thereof include spraying treatment in which a suspension of the present compound C or the composition A is sprayed onto seed surface in the form of mist; smearing treatment in which the present compound C or the composition A is coated a surface of seeds; a soaking treatment in which the seeds are soaked into the solution of the present compound C or the composition A for a certain time; and a method for coating the seeds with a carrier containing the present compound C or the composition A (film coating treatment, pellet coating treatment).

When the composition A is applied to seeds, the composition A may be also applied to seeds as a single formulation, or the composition A may be applied to seeds as a divided plural of formulations by a plurality of times. Examples of the method in which the composition A is applied as a divided plural of formulations by a plurality of times include, for exmaple, a method in which the formulations comprising as an active component the present compound C only are applied, and seeds are air dried, followed by applying the formulations comprising the present ingredient: and a method in which the formulations comprising as an active component the present compound C and the present ingredients are applied, and seeds are air dried, followed by applying the formulations comprising the present ingredients other than the already-applied present ingredients, are included.

The above-mentioned soybean may be a plant which can be produced by natural mating, a soybean which can be generated by mutation, a F1 hybrid soybean, and a transgenic soybean (also referred to as genetically modified soybean). In general, these soybeans have characteristics that are tolerance to herbicides, accumulation of toxic substances against pests (which is also referred to as pest resistance), suppression of sensitivity to diseases (which is also referred to as disease resistance), increase of yield potential, improvement of tolerance to biological and abiotic stress factors, modification of quality of products (for example, increase or decrease of the content of ingredient(s), change of composition, or improvement of storability and processability), and the like. Techniques for producing the above-mentioned soybeans include, for example, traditional breed improvement techniques; genetic recombination technologies; genome breeding technologies; new breeding techniques; and genome editing techniques.

Examples of the soybeans which are imparted with herbicide tolerance include auxin type herbicidal compounds such as 2,4-D, dicamba; soybeans having tolerance to glufosinate, soybeans having tolerance to glyphosate, soybeans having tolerance to isoxaflutole, soybeans having tolerance to 4-hydroxyphenylpyruvate dioxygenase inhibitory herbicides (such mesotrione); soybeans having tolerance to imidazolinone type herbicides; acetolactate synthase (ALS) inhibitory herbicides (such as sulfonylurea herbicide inhibitors); and soybeans having tolerance to protoporphyrinogen oxidase inhibitory herbicides (such as flumioxazin), and the others.

The soybeans which are imparted with herbicide tolerance by genetic recombination technologies can be produced by introducing foreign genes (such as genes derived from other organisms such as microorganisms). For example, a tolerance to 2,4-D is introduced by “aad-12” which is a gene derived from Delftia acidovorans; a tolerance to Dicamba is introduced by “dmo” which is a gene derived from Stenotrophomonas maltophilia strain DI-6; a tolerance to glufosinate is introduced by “bar” which is a gene derived from Streptomyces hygroscopicus or “pat” which is a gene derived from Streptomyes viridochromogenes; a tolerance to glyphosate is introduced by “2mepsps” which is a gene derived from Zea mays, “CP4 epsps” which is a gene derived from Agrobacterium tumefaciens strain CP4, or “gat4601” which is a gene derived from Bacillus licheniformis; a tolerance to isoxaflutole is introduced by “hppdPF W336” which is a gene derived from Pseudomonas fluorescens strain A32; a tolerance to mesotrione is introduced by “avhppd-03” which is a gene derived from Oat (Avena sativa); a tolerance to imidazolinone herbicides is introduced by “csr1-2” which is a gene derived from Arabidopsis thaliana; a tolerance to sulfonylurea herbicides is introduced by “gm-hra” which is a gene derived from Glycine max.

Examples of soybeans which are imparted with herbicides by traditional breed improvement techniques or genome breeding technologies include soybean having tolerance to sulfonylurea ALS inhibitory herbicides (such as thifensulfuron methyl) (“STS (registered trademark) soybean”).

Examples of soybeans which are imparted with herbicides by a new breeding technique include the plants in which glyphosate tolerance is imparted to nontransgenic soybean by using Roundup Ready (Registered trademark) having glyphosate tolerance as a rootstock (see, Weed Technology 27: 412-416 2013).

Examples of soybeans which are imparted with pest tolerance include soybean having tolerance to Lepidoptera pests (such as Pseuoplusia includes, Helicoverpa zea, Spodoptera frugiperda), soybean having tolerance to Hemiptera (such as Aphis glycines), and soybean having tolerance to Nematode (such as Heterodera glycines, Meloidogyne incognita).

The soybeans which are imparted with pest tolerance by genetic recombination technologies can be produced by introducing foreign genes (such as genes encoding 5-endotoxin which is insecticidal protein derived from Bacillus thuringiensis For example, a tolerance to Lepidoptera pests is introduced by “cry1Ac” which is a gene derived from Bacillus thuringiensis subsp. Kurstaki strain HD73, “cry1F” which is a gene derived from Bacillus thuringiensis var. aizawai, “cry1A.105” which is a gene derived from Bacillus thuringiensis subsp. kumamotoensis, or “cry2Ab2” which is a gene derived from Bacillus thuringiensis subsp. kumamotoensis.

Examples of soybeans which are imparted with pest tolerance by traditional breed improvement techniques or genome breeding technologies include soybean having as a resistance gene against aphid a resistance gene Rag1 (Tolerance Aphid Gene 1) or a gene Rag1 (Tolerance Aphid Gene 1) and also showing resistance to aphids (see J. Econ. Entomol., 2015, 108, 326.); soybean showing resistance to Heterodera glycines (see Phytopathology, 2016, 106, 1444.); and soybean showing resistance to Spodoptera litura (that is, “Fukuminori”).

Examples of soybeans which are imparted with disease resistance include soybean which is imparted with a resistance to soybean rust disease by traditional breed improvement techniques or genetic recombination technologies. Examples of commonly used resistance genes include, not limited thereto, Rpp1, Rpp2, Rpp3, Rpp4, Rpp5, and Rpp6. These genes may be introduced alone into a soybean, or may be introduced in any combinations of a plural of these genes into soybean. These genes are described in the following scientific documents: Crop Science, 2007, 47, 837.; Theoretical and Applied Genetics, 2008, 117, 57.; Theoretical and Applied Genetics, 117, 545.; Crop Science, 2009, 49, 783.; Theoretical and Applied Genetics, 2009, 119, 271.; Theoretical and Applied Genetics, 2010, 121, 1023.; Theoretical and Applied Genetics, 2012, 125, 133.

Examples of the soybeans which are imparted with disease resistance by genome breeding technologies include soybean showing resistance to soybean stem disease due to Phytophthora sojae by destructing RXLR effector gene (Avr4/6) using CRISPR-Cas9 (see, Mol. Plant. Pathol., 2016, 17, 127.).

Also, soybeans which is imparted with a resistance to soybean diseases other than soybean rust disease (for example, frogeye leaf spot, brown ring spot disease, stem disease, sudden death syndrome) are also included.

Examples of soybeans in which a quality of product is modified by genetic recombination technologies include soybean “Plenish (Trademark)” or “Treus (Trademark)” in which partial gene of 6)-6 desaturase (gm-fad2-1) derived from Glycine max which is the fatty acid desaturase enzyme, is introduced and an expression of the same genes are then suppressed, and the oleic acid contents is enriched; soybean “Vistive Gold (Trademark)”) in which the contents of saturated fatty acid is reduced by introducing genes that produce double-stranded RNA of acyl-acyl carrier protein-thioesterase gene (fatb1-A) derived from Glycine max and genes that produce double-stranded RNA of 5-12 desaturase (fad2-1A) derived from Glycine max; genetically modified soybean in which the contents of stearidonic acid as one of W3 fatty acid is enriched by introducing δ-6 desaturase gene (Pj. D6D) derived from Primula juliae and δ-12 desaturase gene (Nc. Fad3) derived from Neurospora crassa; soybean in which the oil contents is altered; soybean in which the allergen contents is reduced (see U.S. Pat. No. 6,864,362); spybeans in which the lysine contents are increased (see Bio/Technology, 1995, 13, 577.); soybean in which the composition of methionine, leucine, isoleucine, and valine is modified; soybean in which the contents of a sulfur-containing amino acid is increased (see WO 1997/041239 A1); soybean in which the contents of phenolic compound is increased (see US publication No. 2008/235829); soybean in which the contents of vitamin E is increased (see WO 2004/058934 A1).

Examples of soybeans in which a quality of product is modified by genetic recombination technologies include soybean in which the contents of allergen is reduced (that is, “Yumeminori”).

Examples of the plants in which the traits related to plant growth and yields are altered include soybean in which the plant growth is enhanced by introducing a gene derived from thale cress encoding transcription factor which regulates daily periodicity (“bbx32”), and thereby a high yields are expected.

Examples of soybeans having other characteristics include soybean in which an uptake of phosphorus is improved; soybean which is imparted with fertility traits; soybean which is imparted with tolerance to drought; soybean which is imparted with tolerance to low temperature; soybean which is imparted with tolerance to high salinity; soybean in which iron chlorosis is altered; and soybean in which chloride sensitivity is altered.

Examples of the above-mentioned soybeans encompass also soybeans in which two or more characteristics selected from the above-mentioned herbicide tolerance, pest resistance, disease resistance, abiotic stress tolerance, traits relating to growth or yield, traits relating to nutrient intake, traits relating to product quality, or fertility traits are imparted. Examples of these soybeans include soybean having a tolerance to glyphosate; soybean having a tolerance to glyphosate; soybean having tolerance to glufosinate; soybean having a resistance to frogeye leaf spot, Sudden Death Syndrome, southern stem canker, Phytophthora root rot, southern root-knot nematode, Sclerotinia white mold, brown stem rot, or soybean cyst nematode; soybean in which iron chlorosis is improved; and soybean in which chloride sensitivity is altered (that is, “Credenz (registered trademark) soybean”).

Hereinafter, the soybeans that is commercially available or has been developed are listed below. Hereafter, they are described as [Event Name, Event code, Tread name]. Also, NA represents an information that is not existed or is unavailable. Many of these soybeans is listed in a registration database (GM APPROVAL DATABASE) in a website (http://www.isaaa.org/) of INTERNATINAL SERVICE for the ACQUISITION of AGRI-BIOTECH APPLICATIONS, ISAAA).

[260-05(G94-1, G94-19, G168), DD-026005-3, NA], [A2704-12, ACS-GM005-3, Liberty Link (trademark) soybean], [A2704-21, ACS-GM004-2, Liberty Link (trademark) soybean], [A5547-127, ACS-GM006-4, Liberty Link (trademark) soybean], [A5547-35, ACS-GM008-6, Liberty Link (trademark) soybean], [CV127, BPS-CV127-9, Cultivance], [DAS44406-6, DAS-44406-6, NA], [DAS68416-4, DAS-68416-4, Enlist (trademark) Soybean], [DAS68416-4xMON89788, DAS-68416-4xMON-89788-1, NA], [DAS81419, DAS-81419-2, NA], [DAS81419xDAS44406-6, DAS-81419-2xDAS-44406-6, NA], [DP305423, DP-305423-1, Treus (trademark) or Plenish (trademark)], [DP305423xGTS40-3-2, DP-305423-1xMON-04032-6, NA], [DP356043, DP-356043-5, Optimum GAT (trademark)], [FG72(FG072-2,FG072-3), MST-FG072-3, NA], [FG72xA5547-127, MST-FG072-3xACS-GM006-4, NA], [GTS40-3-2(40-3-2), MON-04032-6, Roundup Ready (trademark) soybean], [GU262, ACS-GM003-1, Liberty Link (trademark) soybean], [IND-00410-5, IND-00410-5, Verdeca HB4 Soybean], [MON87701, MON-87701-2, NA], [MON87701xMON89788, MON-87701-2xMON-89788-1, Intacta (trademark) Roundup Ready (trademark) 2 Pro], [MON87705, MON-87705-6, Vistive Gold (trademark)], [MON87705xMON87708, MON-87705-6xMON-87708-9, NA], [MON87705xMON87708xMON89788, MON-87705-6xMON-87708-9xMON-89788-1, NA], [MON87705xMON89788, MON-87705-6xMON-89788-1, NA], [MON87708, MON-87708-9, Genuity (registered trademark) Roundup Ready (trademark) 2 Xtend (trademark)], [MON87708xMON89788, MON-87708-9xMON-89788-1, Roundup Ready 2 Xtend (registered trademark)], [MON87712, MON-87712-4, NA], [MON87751, MON-87751-7, NA], [MON87751xMON87701xMON87708xMON89788, MON-87751-7xMON-87701-2xMON87708xMON89788, NA], [MON87769, MON87769-7, NA], [, MON87769xMON89788, MON-87769-7xMON-89788-1, NA], [MON89788, MON-89788-1, Genuity (registered trademark) Roundup Ready 2 Yield (trademark)], [SYHT0H2, SYN-000H2-5, Herbicide-tolerant Soybean line], [W62, ACS-GM002-9, Liberty Link (trademark) soybean], [W98, ACS-GM001-8, Liberty Link (trademark) soybean], [OT96-15, OT96-15, NA], [NA, NA, STS (registered trademark) soybean], [NA, NA, Credenz (registered trademark) soybean], [NA, NA, Enlist E3 (trademark)], [NA, NA, Enlist (trademark) Roundup Ready 2 Yield (registered trademark)], [NA, NA, Fukuminori], [NA, NA, Yumeminori], [DP305423 x MOV87708, DP-305423-1 x MON-87708-9, NA], [DP305423 x MOV87708 x MON89788, DP-305423-1 x MON-87708-9 x MON-89788-1, NA], [DP305423 x MON89788, DP-305423-1 x MON-89788-1, NA]

An application of the present compound, the present compound C, the present compound D, or the composition A can provide an effect of a promotion of the growth of a plant, such as an increase in the rate of seedling establishment, an increase in the number of healthy leaves, an increase in the height of the plant, an increase in the weight of the plant body, an increase in the leaf area, an increase in the number or weight of seeds, an increase in the number of occasion of flower setting or fruit setting, and a promoted growth of a root and the like. Also, an application of the present compound, the compound of the present invention, or the composition A can provide an increase of a resistance against an abiotic stress such as a temperature stress (for example, high-temperature stress or low-temperature stress), water stress (for example, drought stress or excess water stress), and a salt stress.

The present invention provides a composition having an excellent efficacy for controlling a plant disease, and a method for controlling plant diseases.

The present inventors have intensively studied to find out a composition having an excellent efficacy for controlling a plant disease, and a method for controlling a plant disease using the same composition, and as a result, found that a composition comprising the compound represented by formula (IV) according to the above [15] and one or more compounds selected from Group (B) (hereinafter, referred to as “Composition of the present invention”) has an excellent efficacy for controlling plant diseases.

The composition for controlling plant diseases of the present invention (that is, the composition of the present invention) comprises the above compound represented by formula (IV) (that is, the present compound A) and one or more compounds selected from Group (B) (that is, the present compound B).

The composition for controlling plant diseases of the present invention (that is, the composition of the present invention) can control plant diseases.

Examples of the embodiments of the present composition A include the following compounds.

[Embodiment A1] The present compound A wherein E⁴ represents a phenyl group, or a 2-methyl phenyl group.

[Embodiment A2] The present compound A wherein E⁴ represents a phenyl group, a 3-fluorophenyl group, a 3-methylphenyl group, a 3-methoxyphenyl group, a 3-(trifluoromethoxy)phenyl group, or a 3-phenoxyphenyl group.

[Embodiment A3] The present compound A wherein E⁴ represents a phenyl group, or a 4-methylphenyl group.

[Embodiment A4] The compound according to [Embodiment A1] to [Embodiment A3] or the present compound A wherein A⁴ represents a nitrogen atom, and L⁴ represents NH.

[Embodiment A5] The compound according to [Embodiment A1] to [Embodiment A3] or the present compound A wherein A⁴ represents CH, and L⁴ represents an oxygen atom.

[Embodiment A6] The present compound A wherein E⁴ represents a 2-thiazolyl group, or a 4-(trifluoromethyl)thizol-2-yl group.

[Embodiment A7] The present compound A wherein E⁴ represents a 2-thiazolyl group, a 5-chlorothiazol-2-yl group, a 5-bromothiazol-2-yl group, a 5-(trifluoromethyl)thiazol-2-yl group, a 5-cyclopropylthiazol-2-yl group, a 5-ethynylthiazol-2-yl group, or a (5-cyclopropylethynyl)thiazol-2-yl group.

[Embodiment A8] The present compound A wherein E⁴ represents a 3-thienyl group.

[Embodiment A9] The compound according to [Embodiment A6] to [Embodiment A8] wherein A⁴ represents a nitrogen atom and L⁴ represents NH.

[Embodiment A10] The compound according to [Embodiment A6] to [Embodiment A8] wherein A⁴ represents CH and L⁴ represents an oxygen atom.

[Embodiment A11] The present compound A wherein A⁴ represents CH, L⁴ represents an oxygen atom, and E⁴ represents a phenyl group, a 2-methyl phenyl group, a 3-methylphenyl group, a 4-methylphenyl group, a 3-fluorophenyl group, a 3-methoxyphenyl group, a 3-(trifluoromethoxy)phenyl group, a 3-phenoxyphenyl group, a 3-thienyl group, a 2-thiazolyl group, a 4-(trifluoromethyl)thizol-2-yl group, a 5-chlorothiazol-2-yl group, a 5-bromothiazol-2-yl group, a 5-(trifluoromethyl)thiazol-2-yl group, a 5-cyclopropylthiazol-2-yl group, a 5-ethynylthiazol-2-yl group, or a (5-cyclopropylethynyl)thiazol-2-yl group, or A⁴ represents a nitrogen atom, L⁴ represents NH, and E⁴ represents a 2-methyl phenyl group, or a 3-fluorophenyl group.

[Embodiment A12] The present compound A wherein A⁴ represents CH, L⁴ represents an oxygen atom, and E⁴ represents a phenyl group, a 2-methyl phenyl group, a 3-methylphenyl group, a 4-methylphenyl group, a 3-fluorophenyl group, a 3-methoxyphenyl group, a 3-(trifluoromethoxy)phenyl group, or a 3-phenoxyphenyl group, or
A⁴ represents a nitrogen atom, L⁴ represents NH, and E⁴ represents a 2-methyl phenyl group, or a 3-fluorophenyl group.

[Embodiment A13] The present compound A wherein A⁴ represents CH, L⁴ represents an oxygen atom, and E⁴ represents a 3-thienyl group, a 2-thiazolyl group, a 4-(trifluoromethyl)thizol-2-yl group, a 5-chlorothiazol-2-yl group, a 5-bromothiazol-2-yl group, a 5-(trifluoromethyl)thiazol-2-yl group, a 5-cyclopropylthiazol-2-yl group, a 5-ethynylthiazol-2-yl group, or a (5-cyclopropylethynyl)thiazol-2-yl group.

Examples of the embodiments of the composition of the present invention include the following compositions.

[Embodiment Z1] A composition wherein a weight ratio of the above compound represented by formula (IV) according to [16] and one or more of compounds selected from sub-group (B-1) is within a range of 1:0.01 to 1:100.

[Embodiment Z2] A composition wherein a weight ratio of the above compound represented by formula (IV) according to [17] and one or more of compounds selected from sub-group (B-1) is within a range of 1:0.01 to 1:100.

[Embodiment Z3] A composition wherein a weight ratio of the above compound represented by formula (IV) according to [16] and one or more of compounds selected from sub-group (B-2) is within a range of 1:0.01 to 1:100.

[Embodiment Z4] A composition wherein a weight ratio of the above compound represented by formula (IV) according to [17] and one or more of compounds selected from sub-group (B-2) is within a range of 1:0.1 to 1:10.

[Embodiment Z5] A composition wherein a weight ratio of the above compound represented by formula (IV) according to [16] and one or more of compounds selected from sub-group (B-3) is within a range of 1:0.1 to 1:10.

[Embodiment Z6] A composition wherein a weight ratio of the above compound represented by formula (IV) according to [17] and one or more of compounds selected from sub-group (B-3) is within a range of 1:0.1 to 1:10

[Embodiment Z7] The composition for controlling plant diseases according to the above [15] wherein in the Group (B), the sub-group (B-1) is a group consisting of pyraclostrobin, picoxystrobin, metyltetraprole, fenpicoxamid, and florylpicoxamid, and the sub-group (B-2) is a group consisting of fluxapyroxad, benzovindiflupyr, fluindapyr, pydiflumetofen, 3-(difluoromethyl)-N-(2,3-dihydro-1,1,3-trimethyl-1H-inden-4-yl)-1-methyl-1H-pyrazole-4-carboxamide, the above compound represented by formula (1), the above compound represented by formula (2), and the above compound represented by formula (3), the sub-group (B-3) is a group consisting of prothioconazolea and mefentrifluconazole, the sub-group (B-4) is a group consisting of 3-(2-methylphenyl)butan-2-yl N-[(3-acetoxy-4-methoxypyridin-2-yl)carbonyl]alaninate, 3-(4-fluoro-2-methylphenyl)butan-2-yl N-[(3-acetoxy-4-methoxypyridin-2-yl)carbonyl]alaninate, 3-(4-methoxy-2-methylphenyl)butan-2-yl N-[(3-acetoxy-4-methoxypyridin-2-yl)carbonyl]alaninate, 3-(2,4-dimethylphenyl)butan-2-yl N-[(3-acetoxy-4-methoxypyridin-2-yl)carbonyl]alaninate, 3-(2-methylphenyl)butan-2-yl N-({3-[(2-methylpropanoyl)oxy]-4-methoxypyridin-2-yl}carbonyl)alaninate, 3-(4-fluoro-2-methylphenyl)butan-2-yl N-({3-[(2-methylpropanoyl)oxy]-4-methoxypyridin-2-yl}carbonyl)alaninate, 3-(4-methoxy-2-methylphenyl)butan-2-yl N-({3-[(2-methylpropanoyl)oxy]-4-methoxypyridin-2-yl}carbonyl)alaninate, 3-(2,4-dimethylphenyl)butan-2-yl N-({3-[(2-methylpropanoyl)oxy]-4-methoxypyridin-2-yl}carbonyl)alaninate, 3-(2-methylphenyl)butan-2-yl N-[(3-hydroxy-4-methoxypyridin-2-yl)carbonyl]alaninate, 3-(4-fluoro-2-methylphenyl)butan-2-yl N-[(3-hydroxy-4-methoxypyridin-2-yl)carbonyl]alaninate, 3-(4-methoxy-2-methylphenyl)butan-2-yl N-[(3-hydroxy-4-methoxypyridin-2-yl)carbonyl]alaninate, and 3-(2,4-dimethylphenyl)butan-2-yl N-[(3-hydroxy-4-methoxypyridin-2-yl)carbonyl]alaninate.

[Embodiment Z8] A composition wherein a weight ratio of the above compound represented by formula (IV) according to [15] and one or more of compounds selected from sub-group (B-1) is within a range of 1:0.1 to 1:10.

[Embodiment Z9] A composition wherein a weight ratio of the above compound represented by formula (TV) according to [15] and one or more of compounds selected from sub-group (B-2) is within a range of 1:0.1 to 1:10.

[Embodiment Z10] A composition wherein a weight ratio of the above compound represented by formula (TV) according to [15] and one or more of compounds selected from sub-group (B-3) is within a range of 1:0.1 to 1:10.

[Embodiment Z11] A composition wherein a weight ratio of the above compound represented by formula (TV) according to [15] and one or more of compounds selected from sub-group (B-4) is within a range of 1:0.1 to 1:10.

[Embodiment Z12] A composition wherein a weight ratio of the above compound represented by formula (TV) according to [Embodiment Z7] and one or more of compounds selected from sub-group (B-1) is within a range of 1:0.1 to 1:10.

[Embodiment Z13] A composition wherein a weight ratio of the above compound represented by formula (TV) according to [Embodiment Z7] and one or more of compounds selected from sub-group (B-2) is within a range of 1:0.1 to 1:10.

[Embodiment Z14] A composition wherein a weight ratio of the above compound represented by formula (TV) according to [Embodiment Z7] and one or more of compounds selected from sub-group (B-3) is within a range of 1:0.1 to 1:10.

[Embodiment Z15] A composition wherein a weight ratio of the above compound represented by formula (IV) according to [Embodiment Z7] and one or more of compounds selected from sub-group (B-4) is within a range of 1:0.1 to 1:10.

The present compound B is explained.

Examples of the present compound B include one or more of the compounds selected from the following sub-group (B-1), sub-group (B-2), sub-group (B-3), and sub-group (B-4).

Sub-group (B-1): mitochondrial electron transport chain complex III inhibitor is a group consisting of coumoxystrobin, enoxastrobin, flufenoxystrobin, picoxystrobin, pyraoxystrobin, pyraclostrobin, mandestrobin, pyrametostrobin, triclopyricarb, trifloxystrobin, dimoxystrobinn, fenaminstrobin, metominostrobin, orysastrobin, fluoxastrobin, fenamidone, pyribencarb, metyltetraprole., cyazofamid, amisulbrom, fenpicoxamid, and florylpicoxamid.

Sub-group (B-2): mitochondrial electron transport chain complex II inhibitor is a group consisting of bixafen, fluxapyroxad, benzovindiflupyr, fluindapyr, pydiflumetofen, 3-(difluoromethyl)-N-(2,3-dihydro-1,1,3-trimethyl-1H-inden-4-yl)-1-methyl-1H-pyrazole-4-carboxamide, a compound represented by formula (1) below, a compound represented by formula (2) below, and a compound represented by formula (3) below.

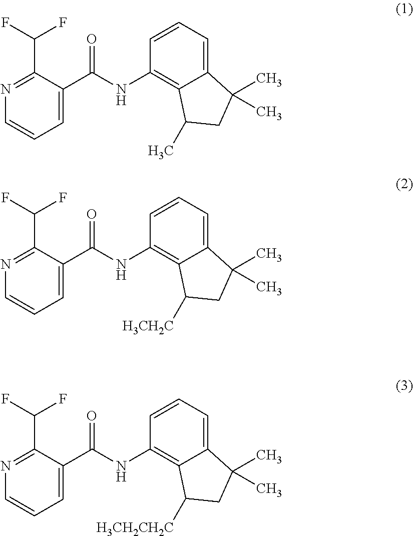

Sub-group (B-3): sterol biosynthesis inhibitor is a group consisting of prothioconazolea and mefentrifluconazole.

Sub-group (B-4): A group consisting of 3-(2-methylphenyl)butan-2-yl N-[(3-acetoxy-4-methoxypyridin-2-yl)carbonyl]alaninate, 3-(4-fluoro-2-methylphenyl)butan-2-yl N-[(3-acetoxy-4-methoxypyridin-2-yl)carbonyl]alaninate, 3-(4-methoxy-2-methylphenyl)butan-2-yl N-[(3-acetoxy-4-methoxypyridin-2-yl)carbonyl]alaninate, 3-(2,4-dimethylphenyl)butan-2-yl N-[(3-acetoxy-4-methoxypyridin-2-yl)carbonyl]alaninate, 3-(2-methylphenyl)butan-2-yl N-({3-[(2-methylpropanoyl)oxy]-4-methoxypyridin-2-yl}carbonyl)alaninate, 3-(4-fluoro-2-methylphenyl)butan-2-yl N-({3-[(2-methylpropanoyl)oxy]-4-methoxypyridin-2-yl}carbonyl)alaninate, 3-(4-methoxy-2-methylphenyl)butan-2-yl N-({3-[(2-methylpropanoyl)oxy]-4-methoxypyridin-2-yl}carbonyl)alaninate, 3-(2,4-dimethylphenyl)butan-2-yl N-({3-[(2-methylpropanoyl)oxy]-4-methoxypyridin-2-yl}carbonyl)alaninate, 3-(2-methylphenyl)butan-2-yl N-[(3-hydroxy-4-methoxypyridin-2-yl)carbonyl]alaninate, 3-(4-fluoro-2-methylphenyl)butan-2-yl N-[(3-hydroxy-4-methoxypyridin-2-yl)carbonyl]alaninate, 3-(4-methoxy-2-methylphenyl)butan-2-yl N-[(3-hydroxy-4-methoxypyridin-2-yl)carbonyl]alaninate, and 3-(2,4-dimethylphenyl)butan-2-yl N-[(3-hydroxy-4-methoxypyridin-2-yl)carbonyl]alaninate.

Coumoxystrobin, enoxastrobin, flufenoxystrobin, picoxystrobin, pyraoxystrobin, pyraclostrobin, mandestrobin, pyrametostrobin, triclopyricarb, trifloxystrobin, dimoxystrobinn, fenaminstrobin, metominostrobin, orysastrobin, fluoxastrobin, fenamidone, pyribencarb, metyltetraprole., cyazofamid, amisulbrom, fenpicoxamid, florylpicoxamid, bixafen, fluxapyroxad, benzovindiflupyr, fluindapyr, pydiflumetofen, 3-(difluoromethyl)-N-(2,3-dihydro-1,1,3-trimethyl-1H-inden-4-yl)-1-methyl-1H-pyrazole-4-carboxamide, prothioconazolea and mefentrifluconazole to be used in the present invention are all known compounds, and are described in, for exmaple, pages 247, 408, 514, 904, 979, 974, 709, 978, 1175, 1180, 376, 444, 779, 832, 530, 442, 993, 789, 256, 45, 466, 498, 126, 558, 97, 515, 969, 652, 966, 728 of “The Pesticide Manual-18th Edition (BCPC published) ISBN 978-9998966-1-4”. These compounds can be obtained from commercially available formulations, or can be prepared by known methods.

The compound represented by formula (1), the compound represented by formula (2), and the compound represented by formula (3) are all known compounds, and described in, for example, WO 2014/095675 A1, and can be prepared by the method described in the publication.

3-(2-Methylphenyl)butan-2-yl N-[(3-acetoxy-4-methoxypyridin-2-yl)carbonyl]alaninate, 3-(4-fluoro-2-methylphenyl)butan-2-yl N-[(3-acetoxy-4-methoxypyridin-2-yl)carbonyl]alaninate, 3-(4-methoxy-2-methylphenyl)butan-2-yl N-[(3-acetoxy-4-methoxypyridin-2-yl)carbonyl]alaninate, 3-(2,4-dimethylphenyl)butan-2-yl N-[(3-acetoxy-4-methoxypyridin-2-yl)carbonyl]alaninate, 3-(2-methylphenyl)butan-2-yl N-({3-[(2-methylpropanoyl)oxy]-4-methoxypyridin-2-yl}carbonyl)alaninate, 3-(4-fluoro-2-methylphenyl)butan-2-yl N-({3-[(2-methylpropanoyl)oxy]-4-methoxypyridin-2-yl}carbonyl)alaninate, 3-(4-methoxy-2-methylphenyl)butan-2-yl N-({3-[(2-methylpropanoyl)oxy]-4-methoxypyridin-2-yl}carbonyl)alaninate, 3-(2,4-dimethylphenyl)butan-2-yl N-({3-[(2-methylpropanoyl)oxy]-4-methoxypyridin-2-yl}carbonyl)alaninate, 3-(2-methylphenyl)butan-2-yl N-[(3-hydroxy-4-methoxypyridin-2-yl)carbonyl]alaninate, 3-(4-fluoro-2-methylphenyl)butan-2-yl N-[(3-hydroxy-4-methoxypyridin-2-yl)carbonyl]alaninate, 3-(4-methoxy-2-methylphenyl)butan-2-yl N-[(3-hydroxy-4-methoxypyridin-2-yl)carbonyl]alaninate, and 3-(2,4-dimethylphenyl)butan-2-yl N-[(3-hydroxy-4-methoxypyridin-2-yl)carbonyl]alaninate are all known compounds, and described in, for example, WO 2019/173665 A1, and can be prepared by the method described in the publication.

In the composition of the present invention, a weight ratio of the present compound A and the present compound B is usually within a range of 1:0.01 to 1:100, preferably 1:0.1 to 1:10.

Though the composition for controlling plant diseases of the present invention may be mere mixtures of the present compound A and the present compound B, the composition of the present invention is usually mixed with the present compound A, and the present compound B, with solid carrier(s), liquid carrier(s), oils and/or surfactant(s), and the others, and if necessary, added by the other auxiliary agents for formulation, to formulate into emulsifiable concentrates, oil solutions, dust formulations, granules, wettable powders, wettable dispersible granules, flowables, dry flowables, microcapsules and the others. In these formulations, the total amount of the present compound A and the present compound B is within a range of usually 0.1 to 100% by weight, preferably 0.2 to 90% by weight, and more preferably 1 to 80% by weight.

Examples of the solid carrier include fine powders or granules of clays (for example, kaolin clay, diatomaceous earth, bentonite, or acid white clay), dry silica, wet silica, hydrated silica, talcs, ceramics, other inorganic minerals (for example, sericite, quartz, sulfur, active carbon, or calcium carbonate); chemical fertilizers (for example, ammonium sulfate, ammonium phosphate, ammonium nitrate, urea, or ammonium chloride); as well as synthetic resins (for example, polyester resins such as polypropylene, polyacrylonitrile, polymethyl methacrylate or polyethylene terephthalate; nylon resins (for example, nylon-6, nylon-11, or nylon-66); polyamide resins; polyvinyl chloride, polyvinylidene chloride, vinyl chloride-propylene copolymers, and the others).

Examples of the above-mentioned liquid carriers include water; alcohols (for example, methanol, ethanol, isopropyl alcohol, butanol, hexanol, benzyl alcohol, ethylene glycol, propylene glycol or phenoxy ethanol); ketones (for Example, acetone, methyl ethyl ketone or cyclohexanone); aromatic hydrocarbons (for example, toluene, xylene, ethyl benzene, dodecyl benzene, phenyl xylyl ethane or methylnaphthalene); aliphatic hydrocarbons (for example, hexane, cyclohexane, kerosene or light oil); esters (for example, ethyl acetate, butyl acetate, isopropyl myristate, ethyl oleate, diisopropyl adipate, diisobutyl adipate or propylene glycol monomethyl ether acetate); nitriles (for Example, acetonitrile or isobutyronitrile); ethers (for example, diisopropyl ether, 1,4-dioxane, ethylene glycol dimethyl ether, diethyleneglycol dimethyl ether, diethylene glycol monomethyl ether, propylene glycol monomethyl ether, dipropylene glycol monomethyl ether or 3-methoxy-3-methyl-1-butanol); acid amides (for example, dimethylformamide or dimethylacetamide); halogenated hydrocarbons (for example, dichloromethane, trichloroethane or carbon tetrachloride); sulfoxides (for example, dimethyl sulfoxide); propylene carbonate; and vegetable oils (for example, soybean oil or cottonseed oil).

Examples of the surfactants include nonionic surfactants such as polyoxyethylenated alkyl ethers, polyoxyethylenated alkyl aryl ethers, and polyethylene glycol fatty acid esters; and anionic surfactants such as alkyl sulfonates, alkylbenzene sulfonates and alkyl sulfates. Specific examples thereof include Nimbus (registered trademark), Assist (registered trademark), Aureo (registered trademark), Iharol (registered trademark), Silwet L-77 (registered trademark), BreakThru (registered trademark), SundanceII (registered trademark), Induce (registered trademark), Penetrator (registered trademark), AgriDex (registered trademark), Lutensol A8 (registered trademark), NP-7 (registered trademark), Triton (registered trademark), Nufilm (registered trademark), Emulgator NP7 (registered trademark), Emulad (registered trademark), TRITON X 45 (registered trademark), AGRAL 90 (registered trademark), AGROTIN (registered trademark), ARPON (registered trademark), EnSpray N (registered trademark), and BANOLE (registered trademark), and the others.

Examples of the other auxiliary agents for formulation include a binder, a dispersant, a colorant, and a stabilizer. Specific examples thereof include casein, gelatin, polysaccharides (for example, starch, gum arabic, cellulose derivatives and alginic acid), lignin derivatives, bentonite, water-soluble synthetic polymers (for example, polyvinyl alcohol, polyvinyl pyrrolidone and polyacrylic acids), acidic isopropyl phosphate, 2,6-di-tert-butyl-4-methylphenol, and a mixture of 2-tert-butyl-4-methoxyphenol and 3-tert-butyl-4-methoxyphenol.

The composition of the present invention may be also prepared by formulating the present compound A and the present compound B respectively by each of the above-mentioned methods, and as needed by diluting the resulting formulations with water, and then mixing the respective formulation or the respective dilution thereof.

The composition of the present invention may further contain one or more kinds of other fungicide and/or pesticide.

The composition of the present invention can control plant diseases which are caused by phytopathogenic microorganism such as fungi, Oomycete, Phytomyxea, and bacteria and the others. Examples of the fungi include Ascomycota, Basidiomycota, Blasocladiomycota, Chytridiomycota, Mucoromycota, and Olpidiomycota. Specific examples thereof include the followings. The descriptions in a parenthesis indicates an academic name of phytopathogenic microorganism that causes each of the disease.

### Rice Diseases:

blast (Pyricularia oryzae), brown spot (Cochliobolus miyabeanus), sheath blight (Rhizoctonia solani), bakanae disease (Gibberella fujikuroi), downy mildew (Sclerophthora macrospora), false blast and head blight (Epicoccum nigrum), and seedling blight (Trichoderma viride, Rhizopus oryzae);

### Wheat Diseases:

powdery mildew (Blumeria graminis), fusarium blight (Fusarium graminearum, Fusarium avenaceum, Fusarium culmorum, Microdochium nivale), stripe rust (Puccinia striiformis), stem rust (Puccinia graminis), leaf rust (Puccinia recondita), snow mould (Microdochium nivale, Microdochium majus), typhula snow blight (Typhula incarnata, Typhula ishikariensis), loose smut (Ustilago tritici), stinking smut (Tilletia caries, Tilletia controversa),
eyespot (Pseudocercosporella herpotrichoides), leaf blotch (Septoria tritici), glume blotch (Stagonospora nodorum), tan spot (Pyrenophora tritici-repentis), rhizoctonia seeding blight (Rhizoctonia solani), take-all disease (Gaeumannomyces graminis), and blast (Pyricularia graminis-tritici);

Barley Diseases:
powdery mildew (Blumeria graminis), fusarium head blight (Fusarium graminearum, Fusarium avenaceum, Fusarium culmorum, Microdochium nivale), stripe rust (Puccinia striiformis), stem rust (Puccinia graminis), dwarf leaf rust (Puccinia hordei), loose smut (Ustilago nuda), scald (Rhynchosporium secalis), net blotch (Pyrenophora teres), spot blotch (Cochliobolus sativus), stripe (Pyrenophora graminea), Ramularia disease (Ramularia collo-cygni), and
rhizoctonia seeding blight (Rhizoctonia solani);

### Corn Diseases:

rust (Puccinia sorghi), southern rust (Puccinia polysora), northern leaf blight (Setosphaeria turcica), tropical rust (Physopella zeae), southern leaf blight (Cochliobolus heterostrophus), anthracnose (Colletotrichum graminicola), gray leaf spot (Cercospora zeae-maydis), eyespot (Kabatiella zeae), phaeosphaeria leaf spot (Phaeosphaeria maydis), diplodia rot (Stenocarpella maydis, Stenocarpella macrospora),
stalk rot (Fusarium graminearum, Fusarium verticilioides, Colletotrichum graminicola),
smut (Ustilago maydis), and Physoderma brown spot and Physoderma stalk rot (Physoderma maydis);

### Cotton Diseases:

anthracnose (Colletotrichum gossypii), grey mildew (Ramularia areola), alternaria leaf spot (Alternaria macrospora, Alternaria gossypii), and black root rot (Thielaviopsis basicola);

### Coffee Diseases:

rust (Hemileia vastatrix), and leaf spot (Cercospora coffeicola);

### Rape Seed Diseases:

sclerotinia rot (Sclerotinia sclerotiorum), gray leaf spot (Alternaria brassicae), root rot (Phoma lingam), and light leaf spot (Pyrenopeziza brassicae);

### Sugar Cane Diseases:

rust (Puccinia melanocephela, Puccinia kuehnii), and smut (Ustilago scitaminea);

### Sunflower Diseases:

rust (Puccinia helianthi), and downy mildew (Plasmopara halstedii);

### Citrus Diseases:

melanose (Diaporthe citri), scab (Elsinoe fawcetti), green mold (Penicillium digitatum), blue mold (Penicillium italicum), Phytophthora rot (Phytophthora parasitica, Phytophthora citrophthora), and aspergillus rot (Aspergillus niger);

### Apple Diseases:

blossom blight (Monilinia mali), valsa canker (Valsa ceratosperma), powdery mildew (Podosphaera leucotricha), alternaria leaf spot (Alternaria alternata apple pathotype), scab (Venturia inaequalis), bitter rot (Glomerella cingulata, Colletotrichum acutatum),
blotch (Diplocarpon mali), ring rot (Botryosphaeria berengeriana), crown rot (Phytophtora cactorum), and rust (Gymnosporangium juniperi-virginianae, Gymnosporangium yamadae);

### Pear Diseases:

scab (Venturia nashicola, Venturia pirina), black spot (Alternaria alternata Japanese pear pathotype), and rust (Gymnosporangium haraeanum);

### Peach Diseases:

brown rot (Monilinia fructicola), scab (Cladosporium carpophilum), Phomopsis rot (Phomopsis sp.), and leaf curl (Taphrina deformans);

### Grapes Diseases:

anthracnose (Elsinoe ampelina), ripe rot (Glomerella cingulata, Colletotrichum acutatum), powdery mildew (Uncinula necator), rust (Phakopsora ampelopsidis), black rot (Guignardia bidwellii), and downy mildew (Plasmopara viticola);

### Japanese Persimmon Diseases:

anthracnose (Gloeosporium kaki, Colletotrichum acutatum), and leaf spot (Cercospora kaki, Mycosphaerella nawae);

### Fig Diseases:

rust (Phakopsora nishidana));

### Diseases of Gourd Family:

anthracnose (Colletotrichum lagenarium), powdery mildew (Sphaerotheca fuliginea), gummy stem blight (Didymella bryoniae), Corynespora leaf spot (Corynespora cassiicola), fusarium wilt (Fusarium oxysporum), downy mildew (Pseudoperonospora cubensis), phytophthora rot (Phytophthora capsici), and damping-off (Pythium sp.);

### Tomato Diseases:

early blight (Alternaria solani), leaf mold (Cladosporium fulvum), Cercospora leaf mold (Pseudocercospora fuligena), late blight (Phytophthora infestans), and powdery mildew (Leveillula taurica);

### Eggplant Diseases:

brown spot (Phomopsis vexans), and powdery mildew (Erysiphe cichoracearum);

### Cruciferous Vegetables Diseases:

alternaria leaf spot (Alternaria japonica), white spot (Cercosporella brassicae), clubroot (Plasmodiophora brassicae), downy mildew (Peronospora parasitica), and white rust (Albugo candida);

### Welsh Onion Disease:

rust (Puccinia allii);

### Soybean Diseases:

purple stain (Cercospora kikuchii), sphaceloma scab (Elsinoe glycines), pod and stem blight (Diaporthe phaseolorum var. sojae), rust (Phakopsora pachyrhizi), target spot (Corynespora cassiicola), anthracnose (Colletotrichum glycines, Colletotrichum truncatum), Rhizoctonia rot (Rhizoctonia solani), septoria brown spot (Septoria glycines), Cercospora leaf spot (Cercospora sojina), stem rot (Sclerotinia sclerotiorum), powdery mildew (Microsphaera diffusa), phytophthora stem and root rot (Phytophthora sojae), downy mildew (Peronospora manshurica), sudden death syndrome (Fusarium virguliforme), red crown rot (Calonectria ilicicola), and Diaporthe/Phomopsis complex (Diaporthe longicolla);

### Kidney Bean Diseases:

stem rot (Sclerotinia sclerotiorum), rust (Uromyces appendiculatus), angular leaf spot (Phaeoisariopsis griseola), and anthracnose (Colletotrichum lindemuthianum), and Fusarium root-rot (Fusarium solani);

### Peanut Diseases:

leaf spot (Cercospora personata), brown leaf spot (Cercospora arachidicola), southern blight (Sclerotium rolfsii), and Cylindrocladium black rot (Calonectria ilicicola);

### Garden Pea Disease:

powdery mildew (Erysiphe pisi), and root rot (Fusarium solani);

### Potato Diseases:

early blight (Alternaria solani), late blight (Phytophthora infestans), Pink rot (Phytophthora erythroseptica), powdery scab (Spongospora subterranea f. sp. subterranea), verticillium wilt (Verticillium albo-atrum, Verticillium dahliae, Verticillium nigrescens), dry rot (Fusarium solani), and potato wart (Synchytrium endobioticum);

### Strawberry Disease:

powdery mildew (Sphaerotheca humuli);

### Tea Diseases:

net blister blight (Exobasidium reticulatum), white scab (Elsinoe leucospila), gray blight (Pestalotiopsis sp.), and anthracnose (Colletotrichum theae-sinensis);

### Tobacco Diseases:

brown spot (Alternaria longipes), anthracnose (Colletotrichum tabacum), blue mold (Peronospora tabacina), and black shank (Phytophthora nicotianae);

### Sugar Beet Diseases:

cercospora leaf spot (Cercospora beticola), leaf blight (Thanatephorus cucumeris), root rot (Thanatephorus cucumeris), and aphanomyces root rot (Aphanomyces cochlioides), and rust (Uromyces betae);

### Rose Diseases:

black spot (Diplocarpon rosae), and powdery mildew (Sphaerotheca pannosa);

### Chrysanthemum Diseases:

leaf blight (Septoria chrysanthemi-indici), and white rust (Puccinia horiana);

### Onion Diseases:

botrytis leaf blight (Botrytis cinerea, Botrytis byssoidea, Botrytis squamosa), gray-mold neck rot (Botrytis allii), and small sclerotial neck rot (Botrytis squamosa);

### Various Crops Diseases:

Botrytis rot (Botrytis cinerea), sclerotinia rot (Sclerotinia sclerotiorum), seedling blight (Pythium aphanidermatum, Pythium irregulare, Pythium ultimum);

### Japanese Radish Disease:

alternaria leaf spot (Alternaria brassicicola);

### Turfgrass Diseases:

dollar spot (Sclerotinia homoeocarpa), brown patch and large patch (Rhizoctonia solani), and pythium bligt (Pythium aphanidermatum);

### Banana Disease:

Sigatoka disease (Mycosphaerella fijiensis, Mycosphaerella musicola);

### Lentils Disease:

ascochyta blight (Ascochyta lentis);

### Chickpea Disease:

ascochyta blight (Ascochyta rabiei);

### Green Pepper Disease:

anthracnose (Colletotrichum scovillei);

### Mango Disease:

anthracnose (Colletotrichum acutatum);

### Fruit Trees Diseases:

white root rot (Rosellinia necatrix), and violet root rot (Helicobasidium mompa);
Postharvest diseases of fruits (for example, apple and pear) Mucor rot diseases (Mucor piriformis);
Seed diseases or diseases in the early stages of the growth of various plants caused by Aspergillus spp., Penicillium spp., Fusarium spp., Gibberella spp., Tricoderma spp., Thielaviopsis spp., Rhizopus spp., Mucor spp., Corticium spp., Phoma spp., Rhizoctonia spp. or Diplodia spp.; and the like;

### Viral Diseases:

Lettuce big-vein disease transmitted by Olpidium brassicae, and viral diseases of several crops transmitted by Polymixa spp. (e.g. Polymyxa betae and Polymyxa graminis);

### Diseases Caused by Bacteria:

bacterial seedling blight of rice (Burkholderia plantarii), bacterial spot of cucumber (Pseudomonas syringae pv. Lachrymans), bacterial wilt of eggplant (Ralstonia solanacearum), canker of citrus (Xanthomonas citri), bacterial soft rot of Chinese cabbage (Erwinia carotovora), scab of potato (Streptomyces scabiei), Goss's wilt of corn (Clavibacter michiganensis), Pierce's disease of grapes, olive and peach (Xylella fastidiosa), and crown gall of Rosacead plants such as apple, peach, cherries (Agrobacterium tumefaciens).

In the above phytopathogenic microorganisms, variations within the species are not particularly limited. Namely, the phytopathogenic microorganisms also include any microorganisms that decrease a sensitivity (which also referred to as “have a resistance”) to a specific fungicide. The decrease of the sensitivity may be attributed to a mutation at a target site (target site mutations), or may be attributed to any factors other than target site mutation (non-target site mutations). Examples of the target site mutations include those wherein amino acid substitution is/are caused at a protein as target site by a mutation of an open reading frame which corresponds to an amino acid sequence of a protein, and those wherein amino acid as a target site is overexpressed by any mutations such as a deletion of suppressor sequence in promotor region, an amplification of an enhancer sequence, and an increase of the copy number of a gene and the others. Examples of the non-target site mutations include an enhancement of efflux function which exhausts extracellularly a fungicide inflowed in a cell by ABC transporter, MFS transporter and the others. Also, a detoxification of a fungicide by a metabolism is included.

Examples of the above-mentioned specific fungicides include nucleic acid synthesis inhibitors (such as phenylamide fungicides, acylamino acid fungicides, DNA topoisomerase type II fungicides), mitosis and cell division-inhibitors (such as MBC fungicides, N-phenylcarbamate fungicides), respiratory inhibitors (such as QoI fungicides, QiI fungicides, SDHI fungicides), inhibitors for amino acid synthesis and protein synthesis (such as anilino pyrimidine fungicides), signal transduction inhibitors (such as phenylpyrrole fungicides, dicarboxyimide fungicides), inhibitors for lipid synthesis and cell membrane synthesis (such as phosphorothioate fungicides, dithiolane inhibitors, aromatic hydrocarbon fungicides, heteroaromatic fungicides, carbamate fungicides), sterol biosynthesis inhibitors (such as DMI fungicides including triazoles, hydroxyanilide fungicides, aminopyrazolinone fungicides), cell wall synthesis inhibitors (such as polyoxin inhibitors, carboxylic amide fungicides), melanin synthesis inhibitors (such as MBI-R fungicides, MBI-D fungicides, MBI-P fungicides), as well as the other fungicides (such as cyanoacetoamide oxime fungicides, phenyl acetamide fungicides).

Examples of the amino acid substitution at target site include the followings.
Cytochrome b: G143A, F129L, G137R, I147V, Y279C, Y279S, M295L, A126T, Y132C, C133Y, G137V, G137A, G137S, M139V, T145F, T145R, T145S, T145C, T145L, T145Y, T148M, T148V, T148L, T148I, T148T, N256Y, N256K, N256I, E272D, E272G, E272Q, W273L, W273F, Y274S, Y274F, L275F, L275S, L275T, L295F or L299F;
Cyp51 protein: A311G, A379G, A381G, A410T, A61V, D107V, D134G, D282E, D411N, E297K, F120L, F219S, F449S, F489L, F495I, G138C/R/S, G312A, G412A, G432S, G434C, G448S, G460D/L, G462A, G464S, G484S, G510C, G54E/K/R/V/W, G54W, H147Y, H303Y, H399P, I145F, I330T, I381V/Δ, I471T, I475T, K142R, K143E, K147Q, K175N, K197N, L50S, L98H, M145L, M220K/I/T/V, M288L, N125I, N178S, N22D, N284H, N513K, P216L, P384S, P394L, Q141H, Q88H, R467K, S188N, S208T, S297T, S405F, S508T, S509T, S524T, S52T, S79T, T289A, T440A, T454P, T469S, V101F, V136A/C/G, V490L, Y121F, Y131F/H, Y132F/H/N, Y134F, Y134F, Y136F, Y137F, Y140F/H, Y145F, Y431C, Y459C/D/N/S/P/A, Y461D/H/S/G/L/N/A, Y463D/H/N, Y491H, S259F, G476S or Y68N;
β-tubulin: H6L/Y, Y50C/N/S, Q134K, A165V, E198A/D/G/K/L/Q/V, F200Y, M257L, F200Y, F167Y, Q73R, or L240F;
SdhB: H277R/Y, P225H/F/L/T, N230I, H272L/R/V/Y, H278Y/R, H249L/N/Y, H273Y, N225I/T, T268I/A, I269V, H242R, H257L or T253I;
SdhC: H134R, P80H/L, A85V, S73P, T90I, I86F, N88S, H154Y/R, K49E, R64K, N75S, G79R, S135R, N87S, H153R, H146R, I29V, N33T, N34T, T79I/N, W80S, A84V, N86K/S/A, G90R, R151T/S, H152R, I161S, G169D or H151R;
SdhD: H133R, H132R, S89P, G109V, D124E/N, H134R, G138V, D145G, I50F, M114V or D129E;
OS-1(Shk1): E753K, G420D, I365N/R/S, V368F, Q369H/P, N373S, T447S, F267L, L290S, T765R, Q777R, T489I, E599K or G736Y;
ERG27: S9G, F26S, P57A, T63I, G170R, V192I, L195F, N196T, A210G, I232M, P238S/A, P250S, P269L, P298A, V309M, A314V, S336C, V365A, E368D, N369D, E375K, A378T, L400F/S, Y408S, F412I/S/V/C, A461S or R496T.

Also, examples of phytopathogenic microorganisms whose sensitivity against fungicides are decreased by overexpressing Cyp51 gene, and host plants therefor include the followings.

Septoria tritici of wheat (Reference literature: Pest Management Science. 2012. 68(7). 1034-1040), Rhynchosporium secalis of barley (Reference literature: Molecular Bilogy and Evolution. 2014. 31(7). 1793-1802), Phakopsora pachyrhizi of soybean (Reference literature: Pest Management Science. 2014. 70(3). 378-388), Venturia inaequalis of apple (Reference literature: Phytopathology. 2016. 106(6). 562-571), Penicillium digitatum of citrus (Reference literature: Applied and Environmental Microbiology. 2000. 66(8). 3421-3426).

The phytopathogenic microorganism which can be controlled by the composition of the present invention may be a plural of the above-mentioned amino acid substitutions. In this case, a plural of the amino acid substitutions may be the identical proteins or the different proteins. Also they may have the non-target site mutations and the target site mutations. For example, phytopathogenic microorganism which causes an amino acid substitution of G143A, F129L and G137R on cytochrome b; phytopathogenic microorganism which has an amino acid substitution of G143A on cytochrome b and also has an amino acid substitution with A311G on Cyp51; phytopathogenic microorganism which has an amino acid substitution of G143A and F129L on cytochrome b and also has an amino acid with A311G on Cyp51; phytopathogenic microorganism which has an amino acid substitution of G143A and F129L on cytochrome b and has an amino acid substitution of H6L/Y on β-tubulin, and further is overexpressed by Cyp51 gene.

Examples of phytopathogenic microorganism having a target site mutation include the followings.
Alternaria alternata which has an amino acid substitution of G143A on cytochrome b;
Alternaria arborescens which has an amino acid substitution of G143A on cytochrome b;
Alternaria solani which has an amino acid substitution of F129L on cytochrome b;
Alternaria tomato which has an amino acid substitution of G143A on cytochrome b;
Botryotinia fuckeliana which has an amino acid substitution of G143A on cytochrome b;
Glomerella graminicola which has an amino acid substitution of G143A on cytochrome b;
Corynespora cassiicola which has an amino acid substitution of G143A on cytochrome b;
Cercospora beticola which has an amino acid substitution of G143A on cytochrome b;
Cercospora sojina which has an amino acid substitution of G143A on cytochrome b;
Cladsporium carpophilum which has an amino acid substitution of G143A on cytochrome b;
Colletotrichum graminicola which has an amino acid substitution of G143A on cytochrome b;
Glomerella cingulata which has an amino acid substitution of G143A on cytochrome b;
Blumeria graminis f. sp. hordei which has an amino acid substitution of G143A on cytochrome b;
Blumeria graminis f. sp. tritici which has an amino acid substitution of G143A on cytochrome b;
Parastagonospora nodorum which has an amino acid substitution of G143A on cytochrome b;
Monographella nivalis which has an amino acid substitution of G143A on cytochrome b;
Microdochium majus, nivale which has an amino acid substitution of G143A on cytochrome b;
Mycosphaerella fijiensis which has an amino acid substitution of G143A on cytochrome b;
Didymella rabiei which has an amino acid substitution of G143A on cytochrome b;
Phakopsora pachyrhizi which has an amino acid substitution of F129L on cytochrome b;
Plasmopara viticola which has an amino acid substitution of F129L or G143A on cytochrome b;
Pleospora allii which has an amino acid substitution of G143A on cytochrome b;
Podosphaera fusca which has an amino acid substitution of G143A on cytochrome b;
Podosphaera xanthii which has an amino acid substitution of G143A on cytochrome b;
Podosphaera xanthii which has an amino acid substitution of G143A on cytochrome b;
Pseudoperonospora cubensis which has an amino acid substitution of G143A on cytochrome b;
Magnaporthe oryzae which has an amino acid substitution of F129L or G143A on cytochrome b;
Pyrenophora teres which has an amino acid substitution of F129L on cytochrome b;
Pyrenophora tritici-repentis which has an amino acid substitution of F129L, G137R or G143A on cytochrome b;
Pythium aphanidermatum which has an amino acid substitution of F129L on cytochrome b;
Thanatephorus cucumeris which has an amino acid substitution of F129L or G143A on cytochrome b;
Ramularia collo-cygni which has an amino acid substitution of G143A on cytochrome b;
Rhynchosporium secalis which has an amino acid substitution of G143A on cytochrome b;
Rhizoctonia solani which has an amino acid substitution of F129L on cytochrome b;
Zymoseptoria tritici which has an amino acid substitution of F129L, G137R or G143A on cytochrome b;
Erysiphe necator which has an amino acid substitution of G143A on cytochrome b;
Venturia inaequalis which has an amino acid substitution of G143A on cytochrome b;
Saccharomyces cerevisiae which has an amino acid substitution of I147V on cytochrome b;
Saccharomyces cerevisiae which has an amino acid substitution of L²⁷⁵F on cytochrome b;
Saccharomyces cerevisiae which has an amino acid substitution of Y279C on cytochrome b;
Saccharomyces cerevisiae which has an amino acid substitution of Y279S on cytochrome b;
Saccharomyces cerevisiae which has an amino acid substitution of M295 on cytochrome b;
Puccinia horiana which has an amino acid substitution of L299F on cytochrome b;
fungi or Oomycota each of which has an amino acid substitution of L299F on cytochrome b;
fungi or Oomycota each of which has an amino acid substitution of A126T on cytochrome b;
fungi or Oomycota each of which has an amino acid substitution of Y132C on cytochrome b;
fungi or Oomycota each of which has an amino acid substitution of C133Y on cytochrome b;
fungi or Oomycota each of which has an amino acid substitution of G137V on cytochrome b;
fungi or Oomycota each of which has an amino acid substitution of G137A on cytochrome b;
fungi or Oomycota each of which has an amino acid substitution of G137S on cytochrome b;
fungi or Oomycota each of which has an amino acid substitution of M139V on cytochrome b;
fungi or Oomycota each of which has an amino acid substitution of T145F on cytochrome b;
fungi or Oomycota each of which has an amino acid substitution of T145R on cytochrome b;
fungi or Oomycota each of which has an amino acid substitution of T145S on cytochrome b;
fungi or Oomycota each of which has an amino acid substitution of T145C on cytochrome b;
fungi or Oomycota each of which has an amino acid substitution of T145L on cytochrome b;
fungi or Oomycota each of which has an amino acid substitution of T145Y on cytochrome b;
fungi or Oomycota each of which has an amino acid substitution of T148M on cytochrome b;
fungi or Oomycota each of which has an amino acid substitution of T148V on cytochrome b;
fungi or Oomycota each of which has an amino acid substitution of T148L on cytochrome b;
fungi or Oomycota each of which has an amino acid substitution of T148I on cytochrome b;
fungi or Oomycota each of which has an amino acid substitution of T148T on cytochrome b;
fungi or Oomycota each of which has an amino acid substitution of N256Y on cytochrome b;
fungi or Oomycota each of which has an amino acid substitution of N256K on cytochrome b;
fungi or Oomycota each of which has an amino acid substitution of N256I on cytochrome b;
fungi or Oomycota each of which has an amino acid substitution of E272D on cytochrome b;
fungi or Oomycota each of which has an amino acid substitution of E272G on cytochrome b;
fungi or Oomycota each of which has an amino acid substitution of E272Q on cytochrome b;
fungi or Oomycota each of which has an amino acid substitution of W273L on cytochrome b;
fungi or Oomycota each of which has an amino acid substitution of W273F on cytochrome b;
fungi or Oomycota each of which has an amino acid substitution of Y274S on cytochrome b;
fungi or Oomycota each of which has an amino acid substitution of Y274F on cytochrome b;
fungi or Oomycota each of which has an amino acid substitution of L275S on cytochrome b;
fungi or Oomycota each of which has an amino acid substitution of L275T on cytochrome b;
fungi or Oomycota each of which has an amino acid substitution of L295F on cytochrome b;
Ajellomyces capsulatus which has an amino acid substitution of Y136F on Cyp51;
Aspergillus flavus which has an amino acid substitution of Y132N, K197N, D282E, M288L, T469S, H399P, D411N or T454P on Cyp51;
Aspergillus fumigatus which has an amino acid substitution of N22D, S52T, G54E/K/R/V/W, Y68N, Q88H, L98H, V101F, Y121F, N125I, G138C/R/S, Q141H, H147Y, P216L, F219S, M220K/I/T/V, T289A, S297T, P394L, Y431C, G432S, G434C, T440A, G448S, Y491H or F495I on Cyp51;
Aspergillus parasiticus which has an amino acid substitution of G54W on Cyp51;
Candida albicans which has an amino acid substitution of A61V, Y132F/H, K143E, S405F, F449S, G464S, R467K or I471T on Cyp51;
Cercospora beticola which has an amino acid substitution of E297K, I330T or P384S on Cyp51;
Blumeria graminis f. sp. hordei which has an amino acid substitution of Y136F, K147Q or S509T on Cyp51;
Blumeria graminis f. sp. tritici which has an amino acid substitution of S79T, Y136F, or K175N on Cyp51;
Filobasidiella neoformans which has an amino acid substitution of Y145F or G484S on Cyp51;
Monilinia fructicola which has an amino acid substitution of Y136F on Cyp51;
Mycosphaerella fijiensis which has an amino acid substitution of Y136F, A313G, A381G, Y461D, G462A or Y463D/H/N on Cyp51;
Phakopsora pachyrhizi which has an amino acid substitution of F120L, Y131F/H, K142R, I145F or I475T on Cyp51;
Puccinia triticina which has an amino acid substitution of Y134F on Cyp51;
Pyrenophora teres triticina which has an amino acid substitution of F489L on Cyp51;
Pyrenopeziza brassicae which has an amino acid substitution of S508T on Cyp51;
Saccharomyces cerevisiae which has an amino acid substitution of Y140F/H on Cyp51;
Zymoseptoria tritici which has an amino acid substitution of L50S, D107V, D134G, V136A/C/G, Y137F, M145L, N178S, S188N, S208T, N284H, H303Y, A311G, G312A, A379G, I381V/L, A410T, G412A, Y459C/D/N/S/P/A, G460D/A, Y461D/H/S, V490L, G510C, N513K or S524T on Cyp51;
Erysiphe necator which has an amino acid substitution of Y136F on Cyp51;
Emericella nidulans which has an amino acid substitution of H6L/Y, Y50N/S, Q134K, A165V, E198D/K/Q, F200Y or M257L on β-tubulin;
Botryotinia fuckeliana which has an amino acid substitution of E198A/G/K/V or F200Y on β-tubulin;
Cochliobolus heterostrophus which has an amino acid substitution of F167Y on β-tubulin;
Cercospora beticola which has an amino acid substitution of F167Y or E198A on β-tubulin;
Gibberella fujikuroi which has an amino acid substitution of Y50N, E198V or F200Y on β-tubulin;
Gibberella zeae which has an amino acid substitution of Y50C, Q73R, F167Y, E198K/L/Q or F200Y on β-tubulin;
Helminthosporium solani which has an amino acid substitution of E198A/Q on β-tubulin;
Hypomyces odoratus which has an amino acid substitution of Y50C on β-tubulin;
Parastagonospora nodorum which has an amino acid substitution of H6Y on β-tubulin;
Monilinia fructicola which has an amino acid substitution of H6Y or E198A/K on β-tubulin;
Monilinia laxa which has an amino acid substitution of H6Y or L240F on β-tubulin;
Microdochium majus which has an amino acid substitution of E198A on β-tubulin;
Mycosphaerella fijiensis which has an amino acid substitution of E198A on β-tubulin;
Neurospora crassa which has an amino acid substitution of F167Y or E198G on β-tubulin;
Penicillium aurantiogriseum which has an amino acid substitution of E198A/K or F200Y on β-tubulin;
Penicillium expansum which has an amino acid substitution of F167Y or E198A/K/V on β-tubulin;
Penicillium italicum which has an amino acid substitution of E198K or F200Y on β-tubulin;
Pyrenopeziza brassicae which has an amino acid substitution of L²⁴⁰F on β-tubulin;
Rhynchosporium secalis which has an amino acid substitution of E198G/K or F200Y on I-tubulin;
Sclerotinia homoeocarpa which has an amino acid substitution of E198A/K on β-tubulin;
Sclerotinia sclerotiorum which has an amino acid substitution of E198A on β-tubulin;
Zymoseptoria tritici which has an amino acid substitution of E198A/G on β-tubulin;
Venturia inaequalis which has an amino acid substitution of E198A/K, F200Y or L240F on β-tubulin;
Alternaria alternata which has an amino acid substitution of H277R/Y on SdhB;
Alternaria solani which has an amino acid substitution of H277R/Y on SdhB;
Botryotinia fuckeliana which has an amino acid substitution of P225H/F/L/T, N230I or H272L/R/V/Y on SdhB;
Corynespora cassiicola which has an amino acid substitution of H278Y/R on SdhB;
Stagonosporopsis cucurbitacearum which has an amino acid substitution of H277R/Y on SdhB;
Eurotium oryzae which has an amino acid substitution of H249L/N/Y on SdhB;
Pyrenophora teres which has an amino acid substitution of H277Y on SdhB;
Sclerotinia sclerotiorum which has an amino acid substitution of H273Y on SdhB;
Zymoseptoria tritici which has an amino acid substitution of N225I/T, H273Y, T268I/A or I269V on SdhB;
Erysiphe necator which has an amino acid substitution of H242R on SdhB;
Ustilago maydis which has an amino acid substitution of H257L on SdhB;
Venturia inaequalis which has an amino acid substitution of T253I on SdhB;
Alternaria alternata which has an amino acid substitution of H134R on SdhC;
Botryotinia fuckeliana which has an amino acid substitution of P80H/L or A85V on SdhC;
Corynespora cassiicola which has an amino acid substitution of S73P on SdhC;
Eurotium oryzae which has an amino acid substitution of T90I on SdhC;
Phakopsora pachyrhizi which has an amino acid substitution of I86F, N88S or H154Y/R on SdhC;
Pyrenophora teres which has an amino acid substitution of K49E, R64K, N75S, G79R, H134R or S135R on SdhC;
Ramularia collo-cygni which has an amino acid substitution of N87S, H146R or H153R on SdhC;
Sclerotinia sclerotiorum which has an amino acid substitution of H146R on SdhC;
Zymoseptoria tritici which has an amino acid substitution of I29V, N33T, N34T, T79I/N, W80S, A84V, N86K/S/A, G90R, R151T/S, H152R or I161S on SdhC;
Erysiphe necator which has an amino acid substitution of G169D on SdhC;
Venturia inaequalis which has an amino acid substitution of H151R on SdhC;
Alternaria alternata which has an amino acid substitution of H133R on SdhD;
Alternaria solani which has an amino acid substitution of H133R on SdhD;
Botryotinia fuckeliana which has an amino acid substitution of H132R on SdhD;
Corynespora cassiicola which has an amino acid substitution of S89P or G109V on SdhD;
Eurotium oryzae which has an amino acid substitution of D124E on SdhD;
Pyrenophora teres which has an amino acid substitution of D124E/N, H134R, G138V or D145G on SdhD;
Sclerotinia sclerotiorum which has an amino acid substitution of H132R or D145G on SdhD;
Zymoseptoria tritici which has an amino acid substitution of I50F, M114V or D129E on SdhD;
Phytophthora capsici which has an amino acid substitution of Q1077K or V1109L/M on CesA3;
Phytophthora drechsleri which has an amino acid substitution of V1109L on CesA3;
Phytophthora infestans which has an amino acid substitution of G1105A/V or V1109L on CesA3;
Plasmopara viticola which has an amino acid substitution of G1105S/V on CesA3;
Pseudoperonospora cubensis which has an amino acid substitution of G1105V/W on CesA3;
Alternaria brassicicola which has an amino acid substitution of E753K on OS-1(Shk1);
Alternaria longipes which has an amino acid substitution of G420D on OS-1(Shk1);
Botryotinia fuckeliana which has an amino acid substitution of I365N/R/S, V368F, Q369H/P, N373S or T447S on OS-1(Shk1);
Pleospora allii which has an amino acid substitution of F267L, L290S, T765R or Q777R on OS-1(Shk1);
Sclerotinia sclerotiorum which has an amino acid substitution of T489I, E599 K or G736Y on OS-1(Shk1);
Botryotinia fuckeliana which has an amino acid substitution of S9G, F26S, P57A, T63I, G170R, V192I, L195F, N196T, A210G, I232M, P238S/A, P250S, P269L, P298A, V309M, A314V, S336C, V365A, E368D, N369D, E375K, A378T, L400F/S, Y408S, F412I/S/V/C, A461S or R496T on ERG27.

Zymoseptoria tritici means the same genus as Septoria tritici.

Examples of the plants which the composition of the present invention can be used include the following plants.

### Crops:

corn (dent corn, flint corn, flour corn, popcorn, glutinous corn, sweet corn), rices (long grain rice, dhort grain rice, medium grain rice, Japonica rice, tropical Japonica rice, paddy rice, dry-land rice, floating rice, direct sowing rice, transplanted rice, glutinous rice), wheat (bread wheat (hard wheat, soft wheat, medium wheat, red wheat, white wheat), macaroni wheat, spelt wheat, club wheat, autumn sowing type thereof, spring sowing type thereof), barley (two-row barley (=beer brewing barley), six-row barley), naked barley, pearl barley, autumn sowing type thereof, spring sowing type thereof), rye (autumn sowing type thereof, spring sowing type thereof), triticale (autumn sowing type thereof, spring sowing type thereof), oat (autumn sowing type thereof, spring sowing type thereof), sorghum, cotton (upland cotton, pima cotton), soybean (determinate type soybean, indeterminate soybean, semi-determinate type soybean), groundnut (peanut), common bean (kidney bean), Lima bean, Azuki bean, cowpea bean, mung bean, urad dal bean, runner bean, rice bean, moth bean, tepary bean, broad bean, pea, chickpea, lentil, lupin bean, pigeon pea, alfalfa, buckwheat, sugar beet, rapeseed, canola (autumn sowing type thereof, spring sowing type thereof), sun flower, sugar cane, tobacco and the others;

### Vegetables:

solanaceous vegetables (eggplant, tomato, pimento, pepper, bell pepper and potato, and the others),
cucurbitaceous vegetables (cucumber, pumpkin, zucchini, water melon, melon, and squash, and the others),
cruciferous vegetables (Japanese radish, white turnip, horseradish, kohlrabi, Chinese cabbage, cabbage, leaf mustard, broccoli and cauliflower, and the others),
asteraceous vegetables (burdock, crown daisy, artichoke and lettuce, and the others),
liliaceous vegetables (green onion, onion, garlic and asparagus, and the others),
ammiaceous vegetables (for example, carrot, parsley, celery and parsnip, and the others),
chenopodiaceous vegetables (spinach and Swiss chard, and the others),
lamiaceous vegetables (Perilla frutescens, mint, basil, and lavender, and the others),
strawberry, sweet potato, Dioscorea japonica, colocasia, and the others;

### Fruits:

pomaceous fruits (apple, pear, Japanese pear, Chinese quince and quince, and the others),
stone fleshy fruits (peach, plum, nectarine, Prunus mume, cherry fruit, apricot and prune, and the other),
citrus fruits (Citrus unshiu, orange, lemon, lime and grapefruit, and the other),
nuts (chestnut, walnuts, hazelnuts, almond, pistachio, cashew nuts and macadamia nuts, and the other),
berry fruits (blueberry, cranberry, blackberry and raspberry, and the other),
grape, kaki persimmon, olive, Japanese plum, banana, coffee, date palm, coconuts,
   and the others;

### Others:

tea, mulberry, flowering plant, roadside trees (for example, ash, birch, dogwood, Eucalyptus, Ginkgo biloba, lilac, maple, Quercus, poplar, Judas tree, Liquidambar formosana, plane tree, zelkova, Japanese arborvitae, fir wood, hemlock, juniper, Pinus, Picea, and Taxus cuspidate), flowers, ornamental foliage plants, sods, and grasses.

The above plants are not limited specifically, as long as they are breeds that are usually cultivated.

The above plants may be also a plant that can be generated by a natural crossbreeding, a plant that can be generated by mutations, an F1 hybrid plant, and a transgenic plant (which is also referred to as a genetically modified crop). In general, these plants have characteristics that are tolerance to herbicides, accumulation of toxic substances against pests (which is also referred to as pest resistance), suppression of sensitivity to diseases (which is also referred to as disease resistance), increase of yield potential, improvement of tolerance to biological and abiotic stress factors, modification of quality of products (for example, increase or decrease of the content of ingredient(s), change of composition, or improvement of storability and processability), and the like.

The method for controlling plant diseases of the present invention (hereinafter, referred to as “control method of the present invention”) is carried out by applying each of an effective amount of the present compound A and the present compound B, to a plant or soil where the plant is grows.

The plants encompasses the whole plant and a particular part of the plant. Examples of the particular part of the plants include stems and leaves, flowers, ears, fruits, trunks, branches, crowns, seeds, bulbs, and seedlings. Bulbs described herein represents discoid stems, corms, rhizomes, tubers, tuberous, seed tubers, and tuberous roots of a plant. In the present control method, the ratio of the applied amounts of the present compound A relative to the applied amounts of the present compound B is within a range of usually 1:0.01 to 1:100, and preferably 1:0.1 to 1:10.

In the control method of the present invention, the present compound A and the present compound B may be applied separately to a plant or soil where the plant grows in the same period, but are usually applied as the present composition in terms of a convenience on applying.

In the control method of the present invention, examples of the method of applying the present compound A and the present compound B include foliage treatment, soil treatment, root treatment, and seed treatment.

The foliage treatment includes, for example, a method of applying the present compounds onto surface of a plant to be grown by a foliar application or a stem application.

The root treatment includes, for example, a method of soaking a whole or a root of the plant into a medicinal solution comprising the present compound A and the present compound B, as well as a method of attaching a solid formulation comprising the present compound A, the present compound B and the solid carrier to a root of the plant.

The soil treatment includes, for example, soil broadcast, soil incorporation, and irrigation of the agent solution to a soil.

The seed treatment includes, for example, an applying of the present composition to a seed of the plant to be prevented from the plant disease. Specific examples of the treatment include spray treatment by spraying a suspension of the present composition in a mist form onto a surface of a seed, smear treatment by applying the wettable powder, the emulsifiable concentrate or the flowable formulation of the present composition with added by small amounts of water or as itself to a seed, immersion treatment by immersing a seed into a solution of the present composition for a certain period of time, film-coating treatment and pellet-coating treatment. Also the present composition can be applied to bulbs of the plant according to a similar method to the above spray treatment and smear treatment.

Each dose of the present compound A and the present compound B in the control method of the present invention may be varied depending on a kind of plant to be treated, a kind or a frequency of an occurrence of a plant disease as a control subject, a dosage form, a treatment period, a treatment method, a treatment site, a climate condition, etc. In case of an application to a foliage of the plant or a soil where the plant is grown, a total amount of the present compound A and the present compound B is usually 1 to 500 g, preferably 2 to 200 g, and more preferably 10 to 100 g, per 1000 m². Also each dose of the present compound A and the present compound B in the treatment for seed is usually 0.001 to 10 g, and preferably 0.01 to 1 g, per 1 kg of seeds.

The emulsifiable concentrate, the wettable powder or the flowable formulation, etc., is usually applied by diluting them with water, and then spreading them. In this case, usually, each concentration of the present compound A and the present compound B contain 0.0005 to 2% by weight, and preferably 0.005 to 1% by weight of the present compound A and the present compound B in total. The dust formulation or the granular formulation, etc., is usually applied as itself without diluting them.

### Examples

Hereinafter, the present invention is explained in more detail by Preparation Examples, Formulation Examples, and Test Examples, however, the present invention should not be limited to only the these Examples.

As used herein, Me represents a methyl group, Et represents an ethyl group, Pr represents a propyl group, i-Pr represents an isopropyl group, Bu represents a butyl group, i-Bu represents an isobutyl group, t-Bu represents a t-butyl group, Pen represents a pentyl group, Hex represents a hexyl group, c-Pr represents a cyclopropyl group, c-Bu represents a cyclobutyl group, c-Pen represents a cyclopentyl group, c-Hex represents a cyclohexyl group, and Ph represents a phenyl group.

When the Ph group has any substituent(s), the substituent(s) is described together with a substitution position before the symbol. For example, 3,4-Me₂-Ph represents a 3,4-dimethylphenyl group.

The preparation examples of the compounds disclosed herein (including the present compound C, the present compound A and the present compound A) are described.

When a physical property of a compound is measured by a liquid chromatography/mass spectrometry analysis (hereinafter, referred to as LCMS), a measured molecular ion value [M+H]⁺ or [M−H]⁻ and a retention time (hereinafter, referred to as RT) is described. The measured condition for liquid chromatography (hereinafter, referred to as LC) and mass spectrometry (hereinafter, referred to as MS) is described below.

### [LC Condition]

Column: L-column2 ODS, inner diameter 4.6 mm, length 30 m m, particle size 3 μm
(Chemicals Evaluation and Research Institute, Japan)
UV measurement wavelength: 254 nm
Mobile phase: A solution: 0.1% aqueous formic acid solution,
B solution: 0.1% formic acid in acetonitrile
Flow Rate: 2.0 mL/min
Pump: LC-20 AD (manufacturing SHIMAZU) 2 umps (high pressure gradient)
Gradient condition: The following concentration gradient shown in [Table LC1] is liquid-transferred.

TABLE 1
[Table LC1]

| Time (min) | A solution (%) | B solution (%) |
|---|---|---|
| 0.01 | 90 | 10 |
| 2.00 | 0 | 100 |
| 4.00 | 0 | 100 |
| 4.01 | 90 | 10 |

### [MS Condition]

Detector: LCMS-2020 (manufacturing SHIMAZU)
Ionization method: DUIS

### Reference Preparation Example 1

To a mixture of sodium hydride (60%, in oil) 0.75 g and THE 40 mL was added 5-btomo-2-methylphenol 4.54 g at 0° C., and the mixture was stirred for 30 minutes. To the resulting mixture was added a mixture of methyl 2-(methoxyimino)-2-(methylsulfonyl)acetate (which was prepared by a method described in WO 99/67209 A1) and THE 25 mL, and the mixture was stirred at room temperature for 1 hour. Water was added to the resulting mixture, and the mixture was extracted with ethyl acetate. The resulting organic layer was dried over anhydrous magnesium, and concentrated under reduced pressure. The resulting residue was subjected to a silica gel column chromatography to obtain an intermediate compound 1 represented by the following formula 0.36 g.

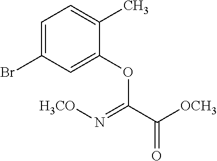 [SMILES: CO/N=C(\Oc1cc(Br)ccc1C)C(=O)OC]

Intermediate compound 1: ¹H-NMR (CDCl₃) δ: 7.15 (1H, d), 7.07 (1H, d), 6.84 (1H, s), 4.05 (3H, s), 3.85 (3H, s), 2.29 (3H, s).

### Reference Preparation Example 2

A mixture of the intermediate compound 1 4.0 g, bis(pinacolato)diboron 5.04 g, [1,1′-bis(diphenylphosphino)ferrocene]palladium (II) dichloride 0.96 g, tripotassium phosphate 8.43 g, and dimethoxyethane 40 mL was stirred at 80° C. under nitrogen atmosphere for 5 hours. The resulting mixture was concentrated under reduced pressure, and the resulting residue was subjected to a silica gel column chromatography to obtain an intermediate compound 2 represented by the following formula.

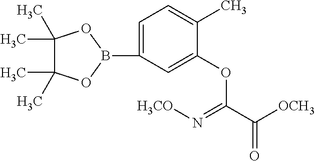 [SMILES: CO/N=C(\Oc1cc(B2OC(C)(C)C(C)(C)O2)ccc1C)C(=O)OC]

Intermediate compound 2: ¹H-NMR (CDCl₃) δ: 7.47 (1H, d), 7.21 (1H, d), 7.11 (1H, s), 4.03 (3H, s), 3.80 (3H, s), 2.37 (3H, s), 1.31 (12H, s).

### Reference Preparation Example 3

To a mixture of methyl 2-(5-bromo-2-methylphenyl)acetate (which was prepared by the method described in WO 2019/016269 A1) 19.4 g, methyl formate 14.7 mL, and dimethoxyethane 150 mL was added potassium tert-butoxide 19.7 g at 0° C., and the mixture was stirred at room temperature for 1 hour. To the resulting mixture was added 1N hydrochloric acid, and the mixture was extracted with MTBE. The resulting mixture was dried over anhydrous magnesium sulfate, and concentrated under reduced pressure. To the resulting residue was added DMF 100 mL, and the mixture was stirred, and thereto were added potassium carbonate 9.9 g, and dimethyl sulfate 6.8 mL at 0° C. successively, and the mixture was stirred at room temperature for 5 hours. Water was added to the resulting mixture, and the mixture was extracted with MTBE. The resulting organic layer was dried over anhydrous magnesium sulfate, and concentrated under reduced pressure. The resulting residue was subjected to a silica gel column chromatography to obtain an intermediate compound 3 represented by the following formula 14.2 g.

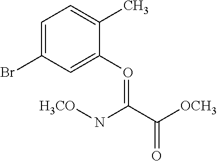

Intermediate compound 3: ¹H-NMR (CDCl₃) δ: 7.56 (1H, s), 7.33 (1H, dd), 7.24 (1H, d), 7.09 (1H, d), 3.84 (3H, s), 3.70 (3H, s), 2.12 (3H, s).

### Reference Preparation Example 3-1

The compounds which were prepared according to the Reference Preparation Example 3 and their physical property value are shown below.

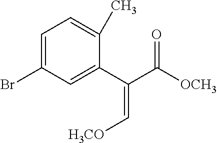 [SMILES: CO/C=C(/C(=O)OC)c1cc(Br)ccc1C]

Intermediate compound 4: ¹H-NMR (CDCl₃) δ: 7.57 (1H, s), 7.38-7.35 (2H, m), 7.29-7.26 (1H, m), 3.87 (3H, s), 3.72 (3H, s).

### Reference Preparation Example 3-2

The compounds which can be prepared according to the Reference Exmaple 3 are shown below.

A compound represented by formula (M1-a):

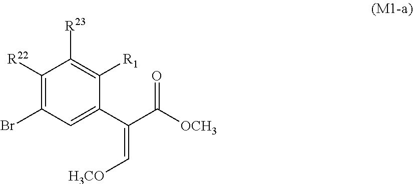

wherein a combination of R¹, R²² and R²³ represent any combinations described in [Table A1].

TABLE A1

| Intermediate | R¹ | R²² | R²³ |
|---|---|---|---|
| 5 | Cl | H | H |
| 6 | F | H | H |
| 8 | Et | H | H |
| 9 | c-Pr | H | H |
| 10 | H | Cl | H |
| 12 | H | H | Cl |

### Reference Preparation Example 3-3

The compounds which were prepared according to the Reference Preparation Example 3 and their physical property value are shown below.

The compounds represented by formula (M1-a) wherein a combination of R¹, R²² and R²³ represent any combinations described in [Table A2].

TABLE A1-2

| Intermediate | R¹ | R²² | R²³ |
|---|---|---|---|
| 7 | OMe | H | H |
| 11 | H | Me | H |
| 13 | H | H | Me |

Intermediate compound 7: ¹H-NMR (CDCl₃) δ: 7.50 (1H, s), 7.37 (1H, dd), 7.28 (1H, d), 6.78 (1H, d), 3.83 (3H, s), 3.76 (3H, s), 3.69 (3H, s).

Intermediate compound 11: ¹H-NMR (CDCl₃) δ: 7.54 (1H, s), 7.51 (1H, s), 7.23-7.15 (2H, m), 3.86 (3H, s), 3.74 (3H, s), 2.38 (3H, s).

Intermediate compound 13: ¹H-NMR (CDCl₃) δ: 7.54 (1H, s), 7.27 (1H, s), 7.24 (1H, s), 7.05 (1H, s), 3.86 (3H, s), 3.74 (3H, s), 2.33 (3H, s).

### Reference Preparation Example 4

A mixture of the intermediate compound 3 3.3 g, bis(pinacolato)diboron 4.7 g, [1,1′-bis(diphenylphosphino)ferrocene]palladium (II) dichloride 0.85 g, tripotassium phosphate 7.4 g, and dimethoxyethane 30 mL was stirred at 80° C. under nitrogen atmosphere for 10 hours. The resulting mixture was concentrated under reduced pressure, and the resulting residue was subjected to a silica gel column chromatography to obtain an intermediate compound 14 represented by the following formula 14.

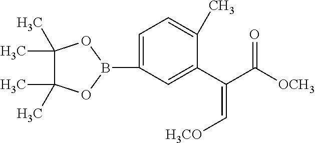 [SMILES: CO/C=C(/C(=O)OC)c1cc(B2OC(C)(C)C(C)(C)O2)ccc1C]

Intermediate compound 14: ¹H-NMR (CDCl₃) δ: 7.67 (1H, dd), 7.55 (1H, s), 7.54 (1H, d), 7.23 (1H, d), 3.80 (3H, s), 3.68 (3H, s), 2.19 (3H, s), 1.32 (12H, s)

### Reference Preparation Example 4-1

The compounds which were prepared according to the Reference Preparation Example 4 and their physical property value are shown below.

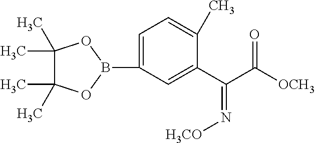 [SMILES: CO/N=C(/C(=O)OC)c1cc(B2OC(C)(C)C(C)(C)O2)ccc1C]

Intermediate compound 14-2: ¹H-NMR (CDCl₃) δ: 7.75 (1H, dd), 7.52 (1H, d), 7.26 (1H, d), 4.04 (3H, s), 3.86 (3H, s), 2.20 (3H, s), 1.33 (12H, s).

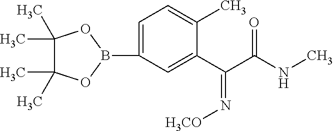

Intermediate compound 14-3: ¹H-NMR (CDCl₃) δ: 7.73 (1H, dd), 7.52 (1H, d), 7.24 (1H, d), 6.72 (1H, d), 3.94 (3H, s), 2.93 (3H, d), 2.20 (3H, s), 1.31 (12H, s).

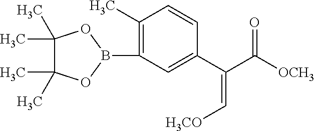 [SMILES: CO/C=C(/C(=O)OC)c1ccc(C)c(B2OC(C)(C)C(C)(C)O2)c1]

Intermediate compound 14-4: ¹H-NMR (CDCl₃) δ: 7.68 (1H, d), 7.52 (1H, s), 7.24 (1H, dd), 7.15 (1H, d), 3.82 (3H, s), 3.71 (3H, s), 2.53 (3H, s), 1.32 (12H, s).

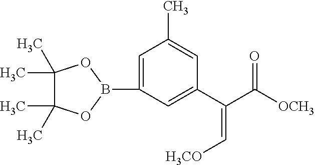 [SMILES: CO/C=C(/C(=O)OC)c1cc(C)cc(B2OC(C)(C)C(C)(C)O2)c1]

Intermediate compound 14-5: ¹H-NMR (CDCl₃) δ: 7.56 (1H, s), 7.54-7.51 (2H, m), 7.19 (1H, s), 3.83 (3H, s), 3.71 (3H, s), 2.35 (3H, s), 1.33 (12H, s).

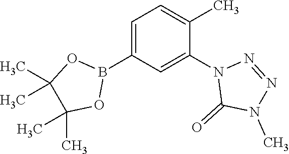 [SMILES: Cc1ccc(B2OC(C)(C)C(C)(C)O2)cc1-n1nnn(C)c1=O]

Intermediate compound 14-6: ¹H-NMR (CDCl₃) δ: 7.81 (1H, dd), 7.76 (1H, d), 7.36 (1H, d), 3.72 (3H, s), 2.30 (3H, s), 1.33 (12H, s).

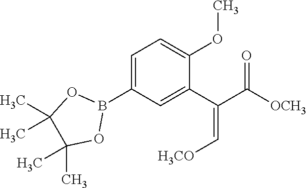 [SMILES: CO/C=C(/C(=O)OC)c1cc(B2OC(C)(C)C(C)(C)O2)ccc1OC]

Intermediate compound 14-7: ¹H-NMR (CDCl₃) δ: 7.77 (1H, dd), 7.60 (1H, d), 7.51 (1H, s), 6.91 (1H, d), 3.81 (3H, s), 3.81 (3H, s), 3.68 (3H, s), 1.32 (12H, s).

### Reference Preparation Example 5

A mixture of the intermediate compound 3 5.0 g, bis(triphenylphosphine)palladium(II) dichloride 0.61 g, tributyl(1-ethoxyvinyl)tin 7.0 mL, and 1,4-dioxane 30 mL was stirred at 100° C. under nitrogen atmosphere for 5 hours. To the resulting mixture was added 1N hydrochloric acid, and the mixture was stirred at room temperature for 30 minutes. The resulting mixture was concentrated under reduced pressure and extracted with ethyl acetate. The resulting organic layers were concentrated under reduced pressure and the residue was subjected to a silica gel column chromatography to obtain an intermediate compound 15 represented by the following formula 3.41 g.

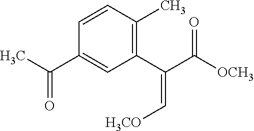 [SMILES: CO/C=C(/C(=O)OC)c1cc(C(C)=O)ccc1C]

Intermediate compound 15: ¹H-NMR (CDCl₃) δ: 7.82 (1H, dd), 7.71 (1H, d), 7.60 (1H, s), 7.31 (1H, d), 3.84 (3H, s), 3.71 (3H, s), 2.57 (3H, s), 2.24 (3H, s)

### Reference Preparation Example 5-1

The compounds which were prepared according to the Reference Preparation Example 5-1 and their physical property value are shown below.

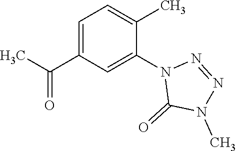 [SMILES: CC(=O)c1ccc(C)c(-n2nnn(C)c2=O)c1]

Intermediate compound 15-1: ¹H-NMR (CDCl₃) δ: ¹H-NMR (CDCl₃) δ: 8.00 (1H, dd), 7.94 (1H, d), 7.48 (1H, d), 3.74 (3H, s), 2.61 (3H, s), 2.37 (3H, s).

### Reference Preparation Example 6

To a mixture of the intermediate compound 15 1.0 g, hydroxylamine hydrochloride salt 0.28 g, and methanol 12 mL was added pyridine 0.64 mL at 0° C., and the mixture was stirred at room temperature for 2.5 hours. The resulting mixture was diluted with ethyl acetate, and washed with water and saturated brine successively. The resulting organic layers were dried over anhydrous sodium sulfate, and concentrated under reduced pressure. The precipitated out solids were washed with a mixed solvent of ethyl acetate and hexane (2:3) to obtain an intermediate compound 16 represented by the following formula 0.91 g.

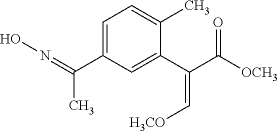 [SMILES: CO/C=C(/C(=O)OC)c1cc(C(C)=NO)ccc1C]

Intermediate compound 16: ¹H-NMR (CDCl₃) δ: 8.78 (1H, s), 7.59 (1H, s), 7.48 (1H, dd), 7.38 (1H, d), 7.23 (1H, d), 3.83 (3H, s), 3.70 (3H, s), 2.26 (3H, s), 2.19 (3H, s).

### Reference Preparation Example 6-1

The compounds which were prepared according to the Reference Preparation Example 6 and their physical property value are shown below.

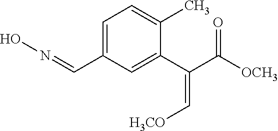 [SMILES: CO/C=C(/C(=O)OC)c1cc(C=NO)ccc1C]

Intermediate compound 17: ¹H-NMR (CDCl₃) δ: 9.01 (1H, br s), 8.11 (1H, s), 7.59 (1H, s), 7.45 (1H, dd), 7.31 (1H, d), 7.23 (1H, d), 3.81 (3H, s), 3.70 (3H, s), 2.19 (3H, s).

### Reference Preparation Example 7

A mixture of 5-bromo-2-methylaniline 12.5 g, triphosgene 29.3 g, and toluene 120 mL was stirred under reflux for 2 hours. The resulting mixture was concentrated under reduced pressure, and to the resulting residue were added 1,1-dimethylhydrazine 5.1 mL and toluene 100 mL under ice-cooling successively, and the mixture was stirred at room temperature for 1.5 hours. The precipitated out solids were filtered, and the filtrates were washed with toluene to obtain an intermediate compound 17 represented by the following formula 13.2 g.

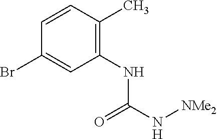

Intermediate compound 17: ¹H-NMR (CDCl₃) δ: 8.23 (1H, d), 8.15 (1H, s), 7.10 (1H, dd), 7.00 (1H, d), 5.42 (1H, s), 2.63 (6H, s), 2.20 (3H, s).

### Reference Preparation Example 8

A mixture of the intermediate compound 17 10 g, triphosgene 22 g, and dichloromethane 350 mL was stirred at 60° C. for 8 hours. The resulting mixture was concentrated under reduced pressure, and water was added to the resulting residue, and the mixture was extracted with ethyl acetate. The resulting organic layers were washed with saturated brine, and dried over anhydrous sodium sulfate, and concentrated under reduced pressure. The resulting residue was subjected to a silica gel column chromatography to obtain an intermediate compound 18 represented by the following formula 3.89 g.

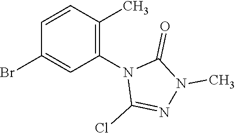 [SMILES: Cc1ccc(Br)cc1-n1c(Cl)nn(C)c1=O]

Intermediate compound 18: ¹H-NMR (CDCl₃) δ: 7.54 (1H, dd), 7.35 (1H, d), 7.25 (1H, d), 3.54 (3H, s), 2.17 (3H, s).

### Reference Preparation Example 9

At room temperature, to a mixture of the intermediate compound 18 1.0 g and THE 15 mL was added sodium methoxide (28% methanol solution) 2.0 mL, and the mixture was stirred under reflux for 1 hour. To the resulting mixture was added aqueous saturated ammonium chloride solution, and the mixture was extracted with ethyl acetate. The resulting organic layers were washed with saturated brine, dried over anhydrous sodium sulfate, and concentrated under reduced pressure. The precipitated out solids were washed with a mixed solvent of ethyl acetate and hexane (2:3) to obtain an intermediate compound 19 represented by the following formula 0.71 g.

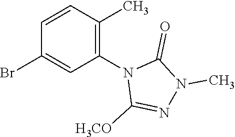 [SMILES: COc1nn(C)c(=O)n1-c1cc(Br)ccc1C]

Intermediate compound 19: ¹H-NMR (CDCl₃) δ: 7.47 (1H, dd), 7.35 (1H, d), 7.20 (1H, d), 3.95 (3H, s), 3.44 (3H, s), 2.17 (3H, s).

### Reference Preparation Example 10

A mixture of the intermediate compound 2 1.5 g, meta chloro perbenzoic acid 1.16 g, ethanol 15 mL, acetonitrile 15 ml, and water 15 ml was stirred at room temperature for 5 hours. To the resulting mixture was added aqueous sodium thiosulfate solution, and the mixture was concentrated under reduced pressure. To the resulting residue was added aqueous saturated sodium bicarbonate solution, and the mixture was extracted with ethyl acetate. The resulting organic layers were dried over anhydrous magnesium sulfate, concentrated under reduced pressure to obtain an intermediate compound 20 represented by the following formula 1.05 g.

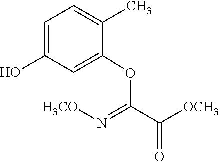 [SMILES: CO/N=C(\Oc1cc(O)ccc1C)C(=O)OC]

Intermediate compound 20: ¹H-NMR (CDCl₃) δ: 7.03 (1H, d), 6.50 (1H, dd), 6.25 (1H, d), 4.64 (1H, s), 4.04 (3H, s), 3.82 (3H, s), 2.25 (3H, s).

### Reference Preparation Example 11

To a mixture of methyl 2-(5-bromo-2-methylphenyl)acetate (which was prepared by the method described in WO 2019/016269 A1) 20.0 g, isoamyl nitrite 13.2 mL, and THE 160 mL was added sodium methoxide (28% methanol solution) 25.4 mL, and the mixture was stirred at room temperature for 7 hours. To the resulting mixture was added 1N hydrochloric acid, and the mixture was extracted with ethyl acetate. The resulting organic layers were washed with water and saturated brine successively, dried over sodium sulfate, and concentrated under reduced pressure. The resulting residue was subjected to a silica gel column chromatography to obtain an intermediate compound represented 21 by the following formula 6.51 g.

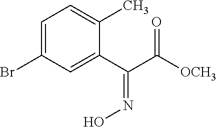

Intermediate compound 21: ¹H-NMR (CDCl₃) δ: 9.25 (1H, s), 7.45 (1H, dd), 7.29 (1H, d), 7.15 (1H, d), 3.87 (3H, s), 2.17 (3H, s).

### Reference Preparation Example 12

To a mixture of the intermediate compound 21 6.51 g, potassium carbonate 4.0 g, and DMF 60 ml was added dimethyl sulfate 2.7 ml at 0° C., and the mixture was stirred at room temperature for 2 hours. To the resulting mixture was added aqueous ammonium chloride solution, and the mixture was extracted with MTBE. The resulting organic layers were washed with water and saturated brine successively, dried over anhydrous sodium sulfate, and concentrated under reduced pressure. The resulting residue was subjected to a silica gel column chromatography to obtain an intermediate compound 22 represented by the following formula 4.83 g.

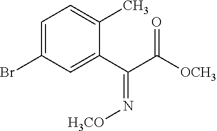 [SMILES: CO/N=C(/C(=O)OC)c1cc(Br)ccc1C]

Intermediate compound 22: ¹H-NMR (CDCl₃) δ: 7.42 (1H, dd), 7.23 (1H, d), 7.12 (1H, d), 4.06 (3H, s), 3.88 (3H, s), 2.13 (3H, s).

### Reference Preparation Example 13

To a mixture of the intermediate compound 22 2.0 g, and methanol 20 ml was added methylamine (9.8 M methanol solution) 1.2 mL, and the mixture was stirred at room temperature overnight. Water was added to the resulting mixture, and the mixture was extracted with ethyl acetate. The resulting organic layers were dried over anhydrous sodium sulfate, and concentrated under reduced pressure. The resulting residue was subjected to a silica gel column chromatography to obtain an intermediate compound represented 23 by the following formula 1.99 g.

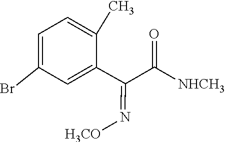 [SMILES: CNC(=O)/C(=N/OC)c1cc(Br)ccc1C]

Intermediate compound 23: ¹H-NMR (CDCl₃) δ: 7.40 (1H, dd), 7.22 (1H, d), 7.10 (1H, d), 6.78 (1H, br s), 3.97 (3H, s), 2.94 (3H, d), 2.12 (3H, s).

### Reference Preparation Example 14

To a mixture of hydrazine monohydrate 15 mL and ethanol 20 mL was added a mixture of 4-acetylindane 2.0 g, acetic acid 0.2 mL and ethanol 17 mL, and the mixture was stirred at 40° C. for 4 hours. Water was added to the resulting mixture, and the mixture was extracted with ethyl acetate. The resulting organic layers were dried over anhydrous sodium sulfate, and concentrated under reduced pressure. The resulting residue was subjected to a silica gel column chromatography to obtain an intermediate compound 24 represented by the following formula 1.65 g.

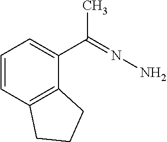

Intermediate compound 24: ¹H-NMR (CDCl₃) δ: 7.20-7.12 (3H, m), 5.29 (2H, s), 3.03 (2H, t), 2.90 (2H, t), 2.13 (3H, s), 2.07-1.99 (2H, m).

### Reference Preparation Example 15

A mixture of the intermediate compound 14 7.4 g, meta chloro perbenzoic acid (purity 70%, containing 30% water) 6.0 g, acetonitrile 30 mL, ethanol 30 mL, and water 30 mL was stirred at room temperature for 25 minutes. To the resulting mixture were added aqueous sodium thiosulfate solution and aqueous sodium hydrogencarbonate solution successively, and the mixture was stirred at room temperature for 1 hour. The resulting mixture was concentrated under reduced pressure and extracted with ethyl acetate. The resulting organic layers were concentrated under reduced pressure to obtain an intermediate compound 25 represented by the following formula 5.7 g.

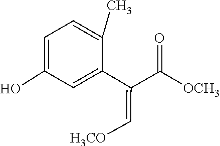 [SMILES: CO/C=C(/C(=O)OC)c1cc(O)ccc1C]

Intermediate compound 25: ¹H-NMR (CDCl₃) δ: 7.54 (1H, s), 7.08 (1H, d), 6.71 (1H, dd), 6.60 (1H, d), 3.83 (3H, s), 3.70 (3H, s), 2.09 (3H, s).

### Reference Preparation Example 15-1

The compounds which were prepared according to the Reference Preparation Example 15 and their physical property value are shown below.

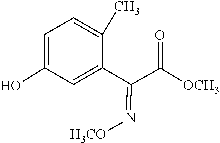 [SMILES: CO/N=C(/C(=O)OC)c1cc(O)ccc1C]

Intermediate compound 25-2: ¹H-NMR (CDCl₃) δ: 7.10 (1H, d), 6.78 (1H, dd), 6.58 (1H, d), 4.94 (1H, br s), 4.05 (3H, s), 3.87 (3H, s), 2.10 (3H, s).

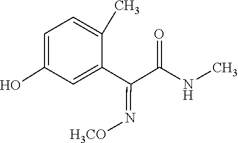 [SMILES: CNC(=O)/C(=N/OC)c1cc(O)ccc1C]

Intermediate compound 25-3: ¹H-NMR (CDCl₃) δ: 7.05 (1H, d), 6.81 (1H, s), 6.70 (1H, dd), 6.51 (1H, d), 5.76 (1H, br s), 3.96 (3H, s), 2.95 (3H, d), 2.07 (3H, s)

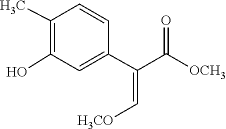 [SMILES: CO/C=C(/C(=O)OC)c1ccc(C)c(O)c1]

Intermediate compound 25-4: ¹H-NMR (CDCl₃) δ: 7.53 (1H, s), 7.10 (1H, d), 6.81 (1H, dd), 6.75 (1H, d), 4.86 (1H, br s), 3.84 (3H, s), 3.73 (3H, s), 2.23 (3H, s)

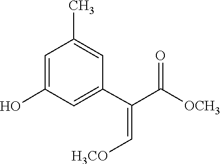 [SMILES: CO/C=C(/C(=O)OC)c1cc(C)cc(O)c1]

Intermediate compound 25-5: ¹H-NMR (CDCl₃) δ: 7.53 (1H, s), 6.70 (1H, s), 6.62-6.57 (2H, m), 3.85 (3H, s), 3.73 (3H, s), 2.30 (3H, s).

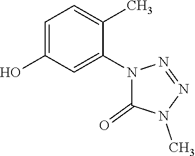

Intermediate compound 25-6: ¹H-NMR (CDCl₃) δ: 7.16 (1H, d), 6.84-6.78 (2H, m), 3.74 (3H, s), 2.17 (3H, s).

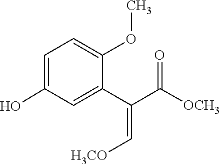 [SMILES: CO/C=C(/C(=O)OC)c1cc(O)ccc1OC]

Intermediate compound 25-7: ¹H-NMR (CDCl₃) δ: 7.49 (1H, s), 6.81-6.73 (2H, m), 6.68 (1H, d), 3.82 (3H, s), 3.73 (3H, s), 3.70 (3H, s).

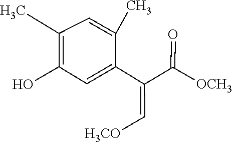 [SMILES: CO/C=C(/C(=O)OC)c1cc(O)c(C)cc1C]

Intermediate compound 25-8: ¹H-NMR (CDCl₃) δ: 7.54 (1H, s), 6.98 (1H, s), 6.55 (1H, s), 4.50 (1H, s), 3.83 (3H, s), 3.70 (3H, s), 2.21 (3H, s), 2.07 (3H, s).

### Reference Preparation Example 16

To a mixture of 5-bromo-2-methyl benzoic acid 10.0 g, DMSO 0.3 mL, and THE 30 mL was added dropwise oxalyl chloride 4.0 mL at 0° C. under nitrogen atmosphere, and the mixture was stirred at room temperature for 1 hour. The resulting mixture was concentrated under reduced pressure to obtain 5-bromo-2-methyl benzoyl chloride.

To a mixture of THE 100 mL and aluminium chloride 12.4 g was added sodium azide 18.1 g at 0° C. under nitrogen atmosphere, and the mixture was stirred under reflux for 40 minutes. To the resulting mixture was added the above-obtained 5-bromo-2-methyl-benzoyl chloride under reflux, and the mixture was stirred under reflux for 90 minutes. After the resulting mixture was cooled to room temperature, aqueous sodium nitrite solution and 1N hydrochloric acid were added successively thereto, and the mixture was extracted with ethyl acetate. The resulting organic layers were dried over sodium sulfate, and concentrated under reduced pressure to obtain an intermediate compound 26 represented by the following formula 11.6 g.

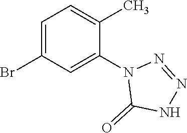

Intermediate compound 26: LCMS: 255 [M+H]⁺, RT=2.16 minutes

### Reference Preparation Example 17

To a mixture of the intermediate compound 26 10.0 g, potassium carbonate 16.3 g, and DMF 60 mL was added dimethyl sulfate 4.8 mL at 0° C., and the mixture was stirred at room temperature for 2 hours. To the resulting mixture was added aqueous ammonium chloride solution, and the mixture was extracted with ethyl acetate. The resulting organic layers were washed with water and saturated brine successively, and dried over anhydrous sodium sulfate, and concentrated under reduced pressure to obtain an intermediate compound 27 represented by the following formula 9.0 g.

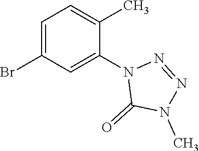

Intermediate compound 27: ¹H-NMR (CDCl₃) δ: 7.54-7.50 (2H, m), 7.24 (1H, d), 3.72 (3H, s), 2.26 (3H, s).

### Reference Preparation Example 18

To a mixture of 5-bromo-2-methylphenyl hydrazine hydrochloride salt 11.1 g and chloroform 110 mL was added dropwise methyl chlorocarbonate 3.6 mL, and the mixture was stirred at room temperature for 1 hour. Water was added to the resulting mixture, and the mixture was extracted with ethyl acetate. The resulting organic layers were washed with saturated brine, dried over anhydrous sodium sulfate, and concentrated under reduced pressure. The resulting residue was subjected to a silica gel column chromatography to obtain an intermediate compound 28 represented by the following formula 8.4 g.

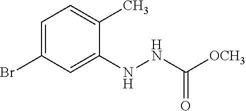

Intermediate compound 28: ¹H-NMR (CDCl₃) δ: 7.02 (1H, d), 6.96-6.90 (2H, m), 6.52 (1H, s), 5.66 (1H, s), 3.78 (3H, s), 2.15 (3H, s).

### Reference Preparation Example 19

To a mixture of the intermediate compound 28 7.0 g, triethylamine 7.5 mL and chloroform 70 mL was added triphosgene 3.2 g at 0° C., and the mixture was stirred at room temperature for 4 hours. Water was added to the resulting mixture, and the mixture was extracted with ethyl acetate. The resulting organic layers were washed with water and saturated brine successively, dried over anhydrous sodium sulfate, and concentrated under reduced pressure to obtain an intermediate compound 29 represented by the following formula 3.5 g.

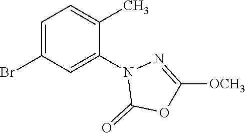 [SMILES: COc1nn(-c2cc(Br)ccc2C)c(=O)o1]

Intermediate compound 29: ¹H-NMR (CDCl₃) δ: 7.54 (1H, d), 7.43 (1H, dd), 7.18 (1H, d), 4.06 (3H, s), 2.31 (3H, s).

### Reference Preparation Example 20

A mixture of the intermediate compound 22 3.5 g, hydroxylamine hydrochloride salt 1.3 g, potassium hydroxide 1.7 g, and methanol 30 mL was stirred at room temperature for 24 hours. To the resulting mixture was added 1N hydrochloric acid, and the mixture was extracted with ethyl acetate. The resulting organic layers were washed with water and saturated brine successively, dried over anhydrous sodium sulfate, and concentrated under reduced pressure to obtain an intermediate compound 30 represented by the following formula 3.1 g.

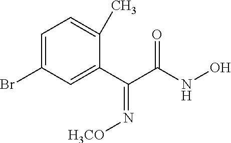 [SMILES: CO/N=C(/C(=O)NO)c1cc(Br)ccc1C]

Intermediate compound 30: ¹H-NMR (CDCl₃) δ: 9.09 (1H, s), 7.63 (1H, s), 7.44 (1H, dd), 7.25 (1H, d), 7.13 (1H, d), 3.99 (3H, s), 2.13 (3H, s).

### Reference Preparation Example 21

To a mixture of the intermediate compound 30 2.6 g, potassium carbonate 3.8 g, and DMF 30 mL was added 1,2-dibromoethane 1.6 mL at 0° C., and the mixture was stirred at room temperature overnight. Water was added to the resulting mixture, and the mixture was extracted with ethyl acetate. The resulting organic layers were washed with saturated brine, dried over anhydrous sodium sulfate, and concentrated under reduced pressure. The resulting residue was subjected to a silica gel column chromatography to obtain an intermediate compound 31 represented by the following formula 1.4 g.

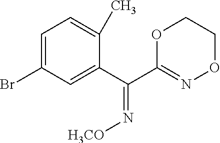 [SMILES: CO/N=C(/C1=NOCCO1)c1cc(Br)ccc1C]

Intermediate compound 31: ¹H-NMR (CDCl₃) δ: 7.38 (1H, dd), 7.21 (1H, d), 7.09 (1H, d), 4.53-4.49 (2H, m), 4.22-4.17 (2H, m), 3.99 (3H, s), 2.16 (3H, s).

### Reference Preparation Example 22

A mixture of the intermediate compound 3 0.56 g, 1-cyclohexen-1-yl boronic acid 0.24 g, [1,1′- [symbol] (diphenylphosphino)ferrocene]palladium (II) dichloride 0.064 g, tripotassium phosphate 0.74 g, water 1 mL, and dimethoxyethane 10 mL was stirred at 80° C. under nitrogen atmosphere for 6 hours. Water was added to the resulting mixture, and the mixture was extracted with ethyl acetate. The resulting organic layers were washed with saturated brine, dried over anhydrous sodium sulfate, and concentrated under reduced pressure. The resulting residue was subjected to a silica gel column chromatography to obtain an intermediate compound 32 represented by the following formula 0.56 g.

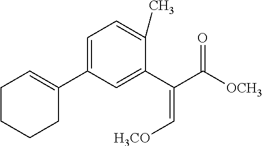 [SMILES: CO/C=C(/C(=O)OC)c1cc(C2=CCCCC2)ccc1C]

Intermediate compound 32: ¹H-NMR (CDCl₃) δ: 7.56 (1H, s), 7.24 (1H, dd), 7.17-7.11 (2H, m), 6.11-6.07 (1H, m), 3.82 (3H, s), 3.70 (3H, s), 2.41-2.36 (2H, m), 2.21-2.16 (2H, m), 2.15 (3H, s), 1.79-1.72 (2H, m), 1.67-1.60 (2H, m).

### Reference Preparation Example 23

A mixture of 2,2′-(4,6-dimethyl-1,3-phenylene)bis(4,4,5,5-tetramethyl-1,3,2-dioxaborolane) 0.61 g, methyl (Z)-2-iodo-3-methoxy-acrylate 0.42 g, 2-(dicyclohexylphoshino)-2′,6′-dimethoxybiphenyl 0.06 g, tris(dibenzylideneacetone)dipalladium (0) 0.08 g, tripotassium phosphate 0.74 g, water 2 mL, and toluene 8 mL was stirred at 100° C. under nitrogen atmosphere for 4 hours. Water was added to the resulting mixture, and the mixture was extracted with ethyl acetate. The resulting organic layers were washed with saturated brine, dried over anhydrous sodium sulfate, and concentrated under reduced pressure. The resulting residue was subjected to a silica gel column chromatography to obtain an intermediate compound 33 represented by the following formula 0.28 g.

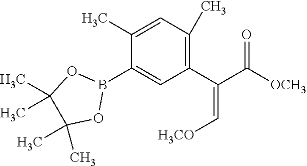 [SMILES: CO/C=C(/C(=O)OC)c1cc(B2OC(C)(C)C(C)(C)O2)c(C)cc1C]

Intermediate compound 33: ¹H-NMR (CDCl₃) δ: 7.54 (1H, s), 7.50 (1H, s), 7.04 (1H, s), 3.80 (3H, s), 3.68 (3H, s), 2.50 (3H, s), 2.14 (3H, s), 1.31 (12H, s).

### Preparation Example 1

A mixture of the intermediate compound 1 0.30 g, PdCl₂ (PPh₃)₂ 0.04 g, 3,3-dimethyl-1-butyne 0.33 g, tetrabutyl ammonium fluoride (1 mol/L THE solution) 3.5 mL and THE 6 mL was stirred at 60° C. under nitrogen atmosphere for 6 hours. To the resulting mixture was added aqueous saturated sodium bicarbonate solution, and the mixture was extracted with ethyl acetate. The resulting organic layers were dried over sodium sulfate, and concentrated under reduced pressure. The residue was subjected to a silica gel column chromatography (ethyl acetate:hexane=1:4) to obtain the present compound 1-1 represented by the following formula 0.08 g.

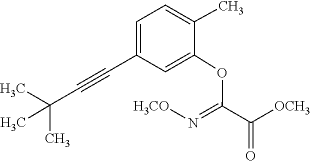 [SMILES: CO/N=C(\Oc1cc(C#CC(C)(C)C)ccc1C)C(=O)OC]

Present compound 1-1: ¹H-NMR (CDCl₃) δ: 7.10-6.98 (2H, m), 6.71 (1H, s), 4.04 (3H, s), 3.82 (3H, s), 2.32 (3H, s), 1.29 (9H, s).

### Preparation Example 1-1

The compounds which were prepared according to the Preparation Example 1 and their physical property value are shown below.

A compound represented by formula (1a):

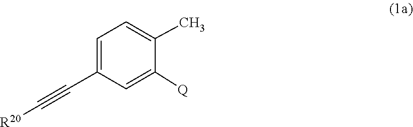

wherein a combination of R²⁰, Q, R² and L² represents any combinations described in [Table A2].

TABLE A2

| Present | | | | |
|---|---|---|---|---|
| Compound | R²⁰ | Q | R³ | L² |
| 1-2 | c-Pr | Q7 | — | O |
| 1-3 | c-Pr | Q3 | OMe | — |
| 1-4 | t-Bu | Q3 | OMe | — |
| 1-5 | c-Pr | Q4 | — | — |
| 1-6 | t-Bu | Q4 | — | — |
| 1-7 | c-Pr | Q5 | — | — |
| 1-8 | t-Bu | Q5 | — | — |
| 1-9 | c-Pr | Q6 | — | — |
| 1-10 | t-Bu | Q6 | — | — |

Present compound 1-2: LCMS: 288 [M+H]⁺, RT=2.15

[symbol]

Present compound 1-3: ¹H-NMR (CDCl₃) δ: 7.33 (1H, dd), 7.23-7.19 (2H, m), 3.93 (3H, s), 3.44 (3H, s), 2.19 (3H, s), 1.46-1.37 (1H, m), 0.91-0.81 (2H, m), 0.81-0.74 (2H, m).

Present compound 1-4: ¹H-NMR (CDCl₃) δ: 7.34 (1H, dd), 7.22-7.21 (2H, m), 3.93 (3H, s), 3.44 (3H, s), 2.18 (3H, s), 1.28 (9H, s).

Present compound 1-5: ¹H-NMR (CDCl₃) δ: 7.40-7.35 (2H, m), 7.25 (1H, d), 3.71 (3H, s), 2.26 (3H, s), 1.47-1.39 (1H, m), 0.90-0.84 (2H, m), 0.82-0.76 (2H, m).

Present compound 1-6: ¹H-NMR (CDCl₃) δ: 7.41-7.37 (2H, m), 7.25 (1H, d), 3.71 (3H, s), 2.27 (3H, s), 1.29 (9H, s).

Present compound 1-7: ¹H-NMR (CDCl₃) δ: 7.39 (1H, d), 7.30 (1H, dd), 7.19 (1H, d), 4.04 (3H, s), 2.32 (3H, s), 1.47-1.39 (1H, m), 0.90-0.76 (4H, m).

Present compound 1-8: ¹H-NMR (CDCl₃) δ: 7.41 (1H, d), 7.31 (1H, dd), 7.19 (1H, d), 4.05 (3H, s), 2.32 (3H, s), 1.30 (9H, s).

Present compound 1-9: ¹H-NMR (CDCl₃) δ: 7.27 (1H, dd), 7.13-7.09 (2H, m), 4.51-4.46 (2H, m), 4.20-4.14 (2H, m), 3.96 (3H, s), 2.18 (3H, s), 1.46-1.37 (1H, m), 0.87-0.80 (2H, m), 0.79-0.73 (2H, m).

Present compound 1-10: ¹H-NMR (CDCl₃) δ: 7.27 (1H, dd), 7.13-7.10 (2H, m), 4.51-4.47 (2H, m), 4.20-4.16 (2H, m), 3.97 (3H, s), 2.19 (3H, s), 1.29 (9H, s).

### Preparation Example 2

A mixture of the intermediate compound 1 0.30 g, 2-fluorophenylboronic acid 0.21 g, [1,1′-bis(diphenylphosphino)ferrocene]palladium (II) dichloride 0.07 g, tripotassium phosphate 0.63 g, dimethoxyethane 15 mL and water 0.1 mL was stirred at 80° C. under nitrogen atmosphere for 5 hours. The resulting mixture was concentrated under reduced pressure, and the residue was subjected to a silica gel column chromatography (ethyl acetate:hexane=1:4) to obtain the present compound 2-1 represented by the following formula 0.10 g.

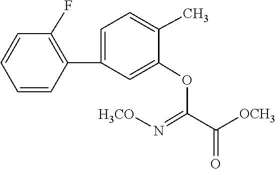 [SMILES: CO/N=C(\Oc1cc(-c2ccccc2F)ccc1C)C(=O)OC]

Present compound 2-1: LCMS: 318 [M+H]⁺. RT=2.15 minutes

### Preparation Example 2-1

The compounds which were prepared according to the Preparation Example 2 and their physical property value are shown below.

A compound represented by formula (1b):

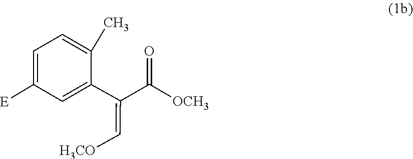

wherein E represents any substituents described in [Table A3].

TABLE A3

| Present | |
|---|---|
| Compound | E |
| 2-2 | 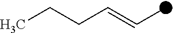 |
| 2-3 | 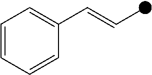 |
| 2-4 | 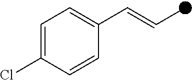 |
| 2-5 | 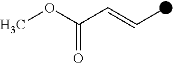 |
| 2-9 | 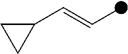 |
| 2-10 | 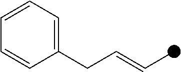 |

Present compound 2-2: LCMS: 275 [M+H]⁺, RT=2.25 minutes

Present compound 2-3: ¹H-NMR (CDCl₃) δ: 7.59 (1H, s), 7.50-7.48 (2H, m), 7.39 (1H, dd), 7.36-7.32 (2H, m), 7.26-7.21 (3H, m), 7.06 (2H, d), 3.84 (3H, s), 3.72 (3H, s), 2.19 (3H, s).

Present compound 2-4: ¹H-NMR (CDCl₃) δ: 7.59 (1H, s), 7.42-7.39 (2H, m), 7.39-7.36 (1H, m), 7.32-7.28 (2H, m), 7.24-7.21 (2H, m), 7.01 (2H, dd), 3.84 (3H, s), 3.72 (3H, s), 2.19 (3H, s).

Present compound 2-5: ¹H-NMR (CDCl₃) δ: 7.67 (1H, d), 7.59 (1H, s), 7.39 (1H, dd), 7.27-7.23 (2H, m), 6.38 (1H, d), 3.84 (3H, s), 3.79 (3H, s), 3.71 (3H, s), 2.20 (3H, s).

Present compound 2-9: ¹H-NMR (CDCl₃) δ: 7.55 (1H, s), 7.18-7.10 (2H, m), 7.03 (1H, d), 6.42 (1H, d), 5.66 (1H, dd), 3.81 (3H, s), 3.69 (3H, s), 2.14 (3H, s), 1.58-1.48 (1H, m), 0.81-0.75 (2H, m), 0.50-0.44 (2H, m).

Present compound 2-10: ¹H-NMR (CDCl₃) δ: 7.55 (1H, s), 7.33-7.27 (2H, m), 7.25-7.18 (4H, m), 7.14 (1H, d), 7.09 (1H, d), 6.41 (1H, d), 6.33-6.24 (1H, m), 3.81 (3H, s), 3.69 (3H, s), 3.52 (2H, d), 2.15 (3H, s).

A compound represented by formula (2b):

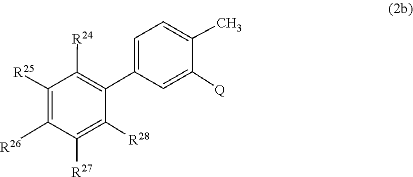

wherein a combination of R²⁴, R²⁵, R²⁶, R²⁷, R²⁸, Q, R³, and L² represents any combinations described in [Table A4].

TABLE A4

| Present | | | | | | | | |
|---|---|---|---|---|---|---|---|---|
| Compound | R²⁴ | R²⁵ | R²⁶ | R²⁷ | R²⁸ | Q | R³ | L² |
| 2-6 | H | H | H | H | H | Q7 | — | O |
| 2-7 | H | H | H | H | H | Q3 | OMe | — |
| 2-8 | F | H | H | H | H | Q3 | OMe | — |
| 2-11 | H | H | H | H | F | Q4 | — | — |
| 2-12 | H | H | H | H | F | Q5 | — | — |
| 2-13 | H | H | H | H | Cl | Q5 | — | — |
| 2-14 | H | H | H | H | F | Q6 | — | — |

Present compound 2-6: ¹H-NMR (CDCl₃) δ: 7.52-7.49 (2H, m), 7.43-7.39 (2H, m), 7.35-7.30 (1H, m), 7.27-7.26 (2H, m), 6.92 (1H, s), 4.05 (3H, s), 3.81 (3H, s), 2.39 (3H, s).

Present compound 2-7: ¹H-NMR (CDCl₃) δ: 7.59-7.55 (3H, m), 7.45-7.37 (4H, m), 7.37-7.31 (1H, m), 3.95 (3H, s), 3.47 (3H, s), 2.26 (3H, s).

Present compound 2-8: ¹H-NMR (CDCl₃) δ: 7.56 (1H, dt), 7.46-7.39 (3H, m), 7.34-7.28 (1H, m), 7.21-7.11 (2H, m), 3.96 (3H, s), 3.47 (3H, s), 2.27 (3H, s).

Present compound 2-11: ¹H-NMR (CDCl₃) δ: 7.63-7.59 (1H, m), 7.55 (1H, s), 7.47-7.42 (2H, m), 7.37-7.30 (1H, m), 7.24-7.12 (2H, m), 3.74 (3H, s), 2.34 (3H, s).

Present compound 2-12: ¹H-NMR (CDCl₃) δ: 7.58 (1H, s), 7.52 (1H, dt), 7.43 (1H, td), 7.37 (1H, d), 7.35-7.29 (1H, m), 7.23-7.11 (2H, m), 4.05 (3H, s), 2.39 (3H, s).

Present compound 2-13: ¹H-NMR (CDCl₃) δ: 7.49-7.45 (2H, m), 7.42 (1H, dd), 7.38-7.25 (4H, m), 4.05 (3H, s), 2.40 (3H, s).

Present compound 2-14: ¹H-NMR (CDCl₃) δ: 7.50 (1H, d), 7.47-7.42 (1H, m), 7.32-7.25 (3H, m), 7.21-7.15 (1H, m), 7.15-7.09 (1H, m), 4.53-4.48 (2H, m), 4.20-4.15 (2H, m), 4.00 (3H, s), 2.27 (3H, s).

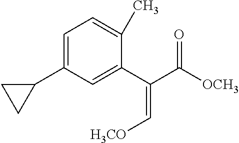 [SMILES: CO/C=C(/C(=O)OC)c1cc(C2CC2)ccc1C]

Present compound 2-15: ¹H-NMR (CDCl₃) δ: 7.55 (1H, s), 7.11 (1H, d), 6.90 (1H, dd), 6.85 (1H, d), 3.82 (3H, s), 3.70 (3H, s), 2.13 (3H, s), 1.89-1.81 (1H, m), 0.93-0.87 (2H, m), 0.68-0.63 (2H, m).

### Preparation Example 3

To a mixture of the sodium hydride (in oil 60%) 0.04 g and DMF 3 mL was added the intermediate compound 16 0.25 g at 0° C., and the mixture was stirred at room temperature for 30 minutes. To the resulting mixture was added (1-bromoethyl)benzene 0.19 mL at 0° C., and the mixture was stirred at room temperature for 2 hours. To the resulting mixture was added aqueous ammonium chloride solution, and the mixture was extracted with ethyl acetate. The resulting organic layers were washed with water and saturated brine, dried over anhydrous sodium sulfate, and concentrated under reduced pressure. The resulting residue was subjected to a silica gel column chromatography (ethyl acetate:hexane=1:4) to obtain the present compound 3-1 represented by the following formula 0.35 g.

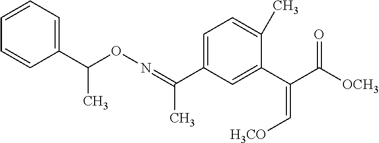 [SMILES: CO/C=C(/C(=O)OC)c1cc(C(C)=NOC(C)c2ccccc2)ccc1C]

Present compound 3-1: ¹H-NMR (CDCl₃) δ: 7.56 (1H, s), 7.48 (1H, dd), 7.39-7.27 (6H, m), 7.17 (1H, d), 5.36 (1H, q), 3.80 (3H, s), 3.68 (3H, s), 2.25 (3H, s), 2.16 (3H, s), 1.60 (3H, d).

### Preparation Example 3-1

The compounds which were prepared according to the Preparation Example 3 and their physical property value are shown below.

A compound represented by formula (1c):

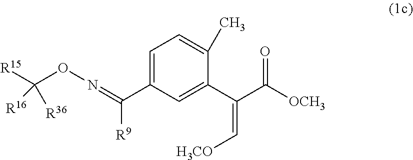

wherein a combination of R⁹ and R¹³ represents a combination described in [Table A5].

TABLE A5

| Present Compound | R⁹ | R¹⁵CR¹⁶R³⁶ |
|---|---|---|
| 3-2 | H | CH(CH₃)Ph |

Present compound 3-2: ¹H-NMR (CDCl₃) δ: 8.09 (1H, s), 7.55 (1H, s), 7.42 (1H, dd), 7.39-7.28 (6H, m), 7.18 (1H, d), 5.33 (1H, q), 3.80 (3H, s), 3.68 (3H, s), 2.16 (3H, s), 1.59 (3H, d).

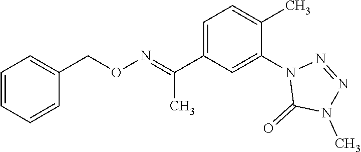 [SMILES: C/C(=N\OCc1ccccc1)c1ccc(C)c(-n2nnn(C)c2=O)c1]

Present compound 3-3: ¹H-NMR (CDCl₃) δ: 7.69 (1H, dd), 7.64 (1H, d), 7.43-7.27 (6H, m), 5.23 (2H, s), 3.70 (3H, s), 2.28 (3H, s), 2.24 (3H, s).

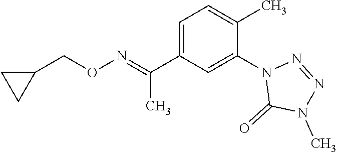 [SMILES: C/C(=N\OCC1CC1)c1ccc(C)c(-n2nnn(C)c2=O)c1]

Present compound 3-4: ¹H-NMR (CDCl₃): 7.70 (1H, dd), 7.64 (1H, d), 7.34 (1H, d), 4.00 (2H, d), 3.72 (3H, s), 2.29 (3H, s), 2.24 (3H, s), 1.27-1.14 (1H, m), 0.59-0.53 (2H, m), 0.34-0.28 (2H, m).

### Preparation Example 4

To a mixture of the intermediate compound 3 0.50 g, phenol 0.25 g and 1,4-dioxane 4 mL were added copper (I) iodide 0.1 g, N,N-dimethylglycine hydrochloride salt 0.22 g, and cesium carbonate 1.14 g successively at room temperature, and the mixture was stirred at 110° C. for 7 hours. Water was added to the resulting mixture, and the mixture was extracted with ethyl acetate. The resulting organic layers were washed with water and saturated brine successively, dried over anhydrous sodium sulfate, and concentrated under reduced pressure. The resulting residue was subjected to a silica gel column chromatography (ethyl acetate:hexane=2:3) to obtain the present compound 4-1 represented by the following formula 0.4 g.

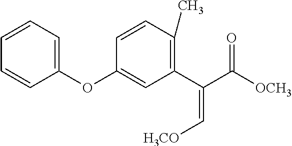 [SMILES: CO/C=C(/C(=O)OC)c1cc(Oc2ccccc2)ccc1C]

Present compound 4-1: ¹H-NMR (CDCl₃) δ: 7.53 (1H, s), 7.32-7.30 (2H, m), 7.17 (1H, d), 7.09-7.00 (3H, m), 6.87 (1H, dd), 6.82 (1H, d), 3.82 (3H, s), 3.70 (3H, s), 2.15 (3H, s).

### Preparation Example 4-1

The compounds which were prepared according to the Preparation Example 4 and their physical property value are shown below.

A compound represented by formula (1d):

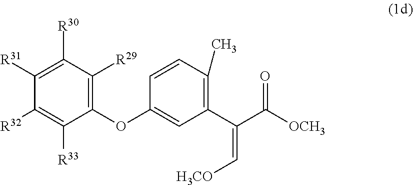

wherein a combination of R²⁹, R³⁰, R³¹, R³², and R³³ represents any combinations described in [Table A6].

TABLE A6

| Present | | | | | |
|---|---|---|---|---|---|
| Compound | R²⁹ | R³⁰ | R³¹ | R³² | R³³ |
| 4-2 | F | H | H | H | H |
| 4-3 | H | H | F | H | H |
| 4-4 | H | Cl | H | H | H |
| 4-5 | Me | H | 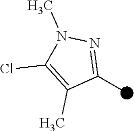 | H | H |
| 4-6 | H | F | H | H | H |
| 4-7 | H | CF₃ | H | H | H |

Present compound 4-2: ¹H-NMR (CDCl₃) δ: 7.54 (1H, s), 7.19-7.11 (2H, m), 7.10-7.00 (3H, m), 6.84 (1H, dd), 6.80 (1H, d), 3.82 (3H, s), 3.70 (3H, s), 2.14 (3H, s).

Present compound 4-3: ¹H-NMR (CDCl₃) δ: 7.53 (1H, s), 7.16 (1H, d), 7.01-6.95 (4H, m), 6.82 (1H, dd), 6.77 (1H, d), 3.81 (3H, s), 3.69 (3H, s), 2.14 (3H, s).

Present compound 4-4: ¹H-NMR (CDCl₃) δ: 7.55 (1H, s), 7.22 (1H, s), 7.20-7.19 (1H, m), 7.04-7.01 (1H, m), 7.00-6.99 (1H, m), 6.91-6.88 (2H, m), 6.82 (1H, d), 3.84 (3H, s), 3.71 (3H, s), 2.17 (3H, s).

Present compound 4-5: ¹H-NMR (CDCl₃) δ: 7.53 (1H, s), 7.51 (1H, d), 7.37 (1H, dd), 7.15 (1H, d), 6.96 (1H, d), 6.82 (1H, dd), 6.72 (1H, d), 3.86 (3H, s), 3.82 (3H, s), 3.70 (3H, s), 2.29 (3H, s), 2.16 (3H, s), 2.14 (3H, s).

Present compound 4-6:1H-NMR (CDCl₃) δ: 7.55 (1H, s), 7.27-7.18 (2H, m), 6.90 (1H, dd), 6.83 (1H, d), 6.81-6.68 (3H, m), 3.83 (3H, s), 3.71 (3H, s), 2.17 (3H, s).

Present compound 4-7: ¹H-NMR (CDCl₃) δ: 7.55 (1H, s), 7.41 (1H, t), 7.33-7.28 (1H, m), 7.25-7.15 (3H, m), 6.90 (1H, dd), 6.82 (1H, d), 3.83 (3H, s), 3.70 (3H, s), 2.17 (3H, s).

A compound represented by formula (2d):

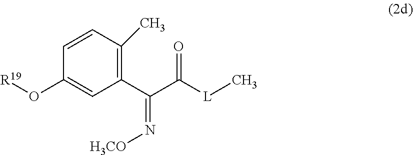

wherein a combination of R¹⁹ and L represents ay combinations described in [Table A7].

TABLE A7

| Present | | |
|---|---|---|
| Compound | R¹⁹ | L |
| 4-8 | 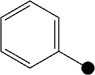 | O |
| 4-9 | 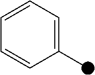 | NH |
| 4-10 | 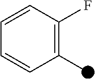 | O |

Present compound 4-8: ¹H-NMR (CDCl₃) δ: 7.36-7.29 (2H, m), 7.20 (1H, d), 7.12-7.06 (1H, m), 7.03-7.00 (2H, m), 6.95 (1H, dd), 6.80 (1H, d), 4.06 (3H, s), 3.87 (3H, s), 2.15 (3H, s).

Present compound 4-9: ¹H-NMR (CDCl₃) δ: 7.34-7.28 (2H, m), 7.18 (1H, d), 7.10-7.05 (1H, m), 7.04-7.00 (2H, m), 6.93 (1H, dd), 6.78 (1H, d), 6.76 (1H, br s), 3.96 (3H, s), 2.92 (3H, d), 2.15 (3H, s).

Present compound 4-10: ¹H-NMR (CDCl₃) δ: 7.21-7.03 (5H, m), 6.91 (1H, dd), 6.78 (1H, d), 4.05 (3H, s), 3.86 (3H, s), 2.15 (3H, s).

### Preparation Example 4-2

The compounds which can be prepared according to the Preparation Exmaple 4 are shown below.

A compound represented by formula (1d) wherein a combination of R²⁹, R³⁰, R³¹, R³², and R³³ represents any combinations described in [Table A8].

TABLE A8

| Present | | | | | |
|---|---|---|---|---|---|
| Compound | R²⁹ | R³⁰ | R³¹ | R³² | R³³ |
| 4-11 | F | H | H | H | F |
| 4-16 | H | Me | H | Me | H |
| 4-17 | H | OCF₃ | H | H | H |
| 4-18 | H | H | OCF₃ | H | H |
| 4-19 | CF₃ | H | H | H | H |
| 4-21 | Cl | H | H | H | H |
| 4-22 | H | H | Cl | H | H |
| 4-23 | F | F | H | H | H |
| 4-24 | F | H | F | H | H |
| 4-25 | F | H | H | F | H |
| 4-26 | H | F | F | H | H |
| 4-27 | H | F | H | F | H |
| 4-28 | OMe | H | H | H | H |
| 4-29 | H | H | OMe | H | H |
| 4-30 | CN | H | H | H | H |
| 4-31 | H | i-Pr | H | H | H |
| 4-32 | H | H | CF₃ | H | H |
| 4-33 | OCF₃ | H | H | H | H |
| 4-34 | H | t-Bu | H | H | H |
| 4-35 | H | Ph | H | H | H |

### Preparation Example 4-3

The compounds which were prepared according to the Preparation Example 4 and their physical property value are shown below.

A compound represented by formula (1d) wherein a combination of R²⁹, R³⁰, R³¹, R³², and R³³ represents any combinations described in [Table A8-2].

TABLE A8-2

| Present | | | | | |
|---|---|---|---|---|---|
| Compound | R²⁹ | R³⁰ | R³¹ | R³² | R³³ |
| 4-12 | Me | H | H | H | H |
| 4-13 | H | Me | H | H | H |
| 4-14 | H | H | Me | H | H |
| 4-15 | H | OMe | H | H | H |
| 4-20 | H | CN | H | H | H |

Present compound 4-12: ¹H-NMR (CDCl₃) δ: 7.52 (1H, s), 7.24-7.09 (3H, m), 7.02 (1H, td), 6.91 (1H, dd), 6.77 (1H, dd), 6.71 (1H, d), 3.81 (3H, s), 3.69 (3H, s), 2.25 (3H, s), 2.13 (3H, s).

Present compound 4-13: ¹H-NMR (CDCl₃) δ: 7.53 (1H, s), 7.18-7.15 (2H, m), 6.88-6.80 (5H, m), 3.81 (3H, s), 3.69 (3H, s), 2.31 (3H, s), 2.15 (3H, s).

Present compound 4-14; 1H-NMR (CDCl₃) δ: 7.53 (1H, s), 7.24-7.20 (1H, m), 6.94-6.90 (2H, m), 6.83 (1H, dd), 6.79 (1H, d), 6.74-6.70 (2H, m), 3.82 (3H, s), 3.70 (3H, s), 2.27 (3H, s), 2.14 (3H, s).

Present compound 4-15: ¹H-NMR (CDCl₃) δ: 7.54 (1H, s), 7.22-7.15 (2H, m), 6.88 (1H, dd), 6.82 (1H, d), 6.64-6.56 (3H, m), 3.82 (3H, s), 3.76 (3H, s), 3.70 (3H, s), 2.15 (3H, s).

Present compound 4-20: ¹H-NMR (CDCl₃) δ: 7.57 (1H, s), 7.34-7.29 (1H, m), 7.25-7.17 (2H, m), 7.09-7.05 (2H, m), 6.89 (1H, dd), 6.82 (1H, d), 3.86 (3H, s), 3.74 (3H, s), 2.18 (3H, s).

### Preparation Example 5

To a mixture of the intermediate compound 19 0.25 g, 2-fluorophenol 0.12 mL, and 1,4-dioxane 4 mL were added copper (I) iodide 0.08 g, N,N-dimethylglycine hydrochloride salt 0.18 g, and cesium carbonate 0.55 g at room temperature, and the mixture was stirred at 110° C. for 7 hours. The resulting mixture was diluted with ethyl acetate, and filtered through Celite. The filtrates were washed with water and saturated brine, dried over anhydrous sodium sulfate, and concentrated under reduced pressure. The resulting residue was subjected to a silica gel column chromatography (ethyl acetate:hexane=1:2) to obtain the present compound 5-1 represented by the following formula 0.05 g.

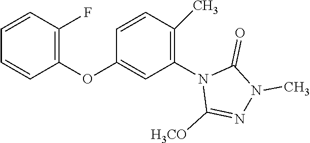 [SMILES: COc1nn(C)c(=O)n1-c1cc(Oc2ccccc2F)ccc1C]

Present compound 5-1: ¹H-NMR (CDCl₃) δ: 7.21-7.08 (5H, m), 6.97 (1H, dd), 6.83 (1H, d), 3.94 (3H, s), 3.43 (3H, s), 2.17 (3H, s).

### Preparation Example 5-1

The compounds which were prepared according to the Preparation Example 5 and their physical property value are shown below.

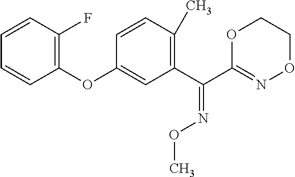 [SMILES: CO/N=C(/C1=NOCCO1)c1cc(Oc2ccccc2F)ccc1C]

Present compound 5-2: LCMS: 345 [M+H]⁺, RT=1.98 minutes

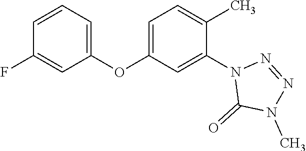 [SMILES: Cc1ccc(Oc2cccc(F)c2)cc1-n1nnn(C)c1=O]

Present compound 5-3: ¹H-NMR (CDCl₃) δ: 7.36-7.25 (2H, m), 7.09 (1H, dd), 7.04 (1H, d), 6.85-6.78 (2H, m), 6.75 (1H, dt), 3.71 (3H, s), 2.28 (3H, s).

A compound represented by formula (2f):

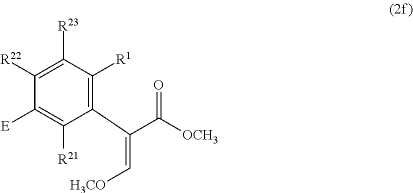

wherein a combination of R¹, R²¹, R²², R²³, and E represents any combinations described in [Table A9].

TABLE A9

| Present | | | | | |
|---|---|---|---|---|---|
| Compound | R¹ | R²¹ | R²² | R²³ | E |
| 7-1 | H | F | H | H | Me |
| 7-2 | F | H | H | H | Me |
| 7-3 | Me | H | OMe | H | Me |
| 7-4 | H | H | H | Me | Me |
| 7-5 | Me | H | H | H | Me |
| 7-6 | H | H | H | H | CF₃ |
| 7-7 | H | H | Me | H | Me |

Present compound 7-1: LCMS: 225 [M+H]⁺, RT=1.85

[symbol]

Present compound 7-2: ¹H-NMR (CDCl₃) δ: 7.58 (1H, s), 7.09-7.05 (1H, m), 7.03 (1H, dd), 6.95 (1H, t), 3.86 (3H, s), 3.73 (3H, s), 2.32 (3H, s).

Present compound 7-3: LCMS: 251 [M+H]⁺, RT=1.88 [symbol] Present compound 7-4: ¹H-NMR (CDCl₃) δ: 7.53 (1H, s), 6.91 (3H, bs), 3.84 (3H, s), 3.73 (3H, s), 2.31 (6H, s).

Present compound 7-5: LCMS: 221 [M+H]⁺, RT=1.89

[symbol]

Present compound 7-6: ¹H-NMR (CDCl₃) δ: 7.61 (2H, bs), 7.53-7.51 (2H, m), 7.47-7.44 (1H, m), 3.89 (3H, s), 3.75 (3H, s).

Present compound 7-7: ¹H-NMR (CDCl₃) δ: 7.53 (1H, s), 7.13-7.03 (3H, m), 3.84 (3H, s), 3.73 (3H, s), 2.26 (3H, s), 2.25 (3H, s).

A compound represented by formula (1h):

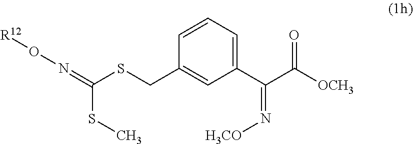

wherein R¹² represents the group described below.

Present compound 8-1 (R¹²=i-Pr): LCMS: 371 [M+H]⁺, RT=2.16 minutes

Present compound 8-2 (R¹²═CH₃CH(CH₃) CH₂): LCMS: 385 [M+H]⁺, RT=2.26 minutes

Present compound 8-3 (R¹²=n-Pen): LCMS: 399 [M+H]⁺, RT=2.35 minutes

A compound represented by formula (1i):

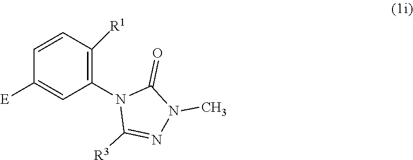

wherein a combination of E, R¹ and R² represents any combinations described in [Table A10].

TABLE A10

| Present | | | |
|---|---|---|---|
| Compound | E | R¹ | R³ |
| 9-1 | 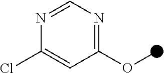 | H | OMe |
| 9-2 | 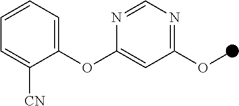 | H | OMe |
| 9-3 | 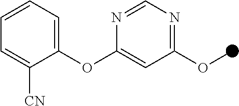 | H | Me |
| 9-4 | 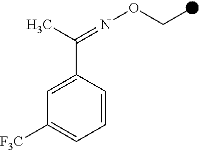 | H | OMe |
| 9-5 | 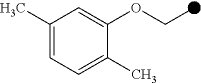 | Me | OMe |

Present compound 9-1: LCMS: 334 [M+H]⁺, RT=1.53 minutes

Present compound 9-2: LCMS: 417 [M+H]⁺, RT=1.70 minutes

Present compound 9-3: LCMS: 401 [M+H]⁺, RT=1.57 minutes

Present compound 9-4: LCMS: 421 [M+H]⁺, RT=2.07 minutes

Present compound 9-5: LCMS: 354 [M+H]⁺, RT=2.01 minutes

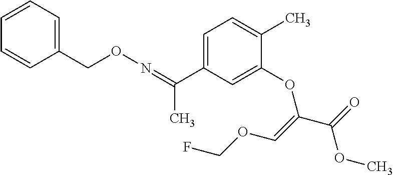 [SMILES: COC(=O)/C(=C/OCF)Oc1cc(C(C)=NOCc2ccccc2)ccc1C]

Compound 9-6: LCMS: 388 [M+H]⁺, RT=2.21 minutes

### Preparation Example 7

To a mixture of sodium hydride 0.03 g and DMF 3 mL was added acetophenone oxime 0.10 g at 0° C., and the mixture was stirred at room temperature for 1 hour. To the resulting mixture was added a mixture of methyl (E)-2-(5-bromomethyl-2-methylphenyl)-3-methoxyacrylate (which was prepared by the method described in WO 1999/028305 A1) 0.30 g and DMF 3 mL, and the mixture was stirred at room temperature for 2 hours. To the resulting mixture was added aqueous ammonium chloride solution, and the mixture was extracted with MTBE. The resulting organic layers were washed with water and saturated brine successively, dried over anhydrous sodium sulfate, and concentrated under reduced pressure. The resulting residue was subjected to a silica gel column chromatography (hexane: ethyl acetate=1:4) to obtain the present compound 10-1 represented by the following formula 0.07 g.

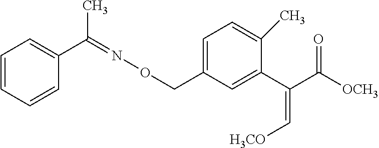 [SMILES: CO/C=C(/C(=O)OC)c1cc(CON=C(C)c2ccccc2)ccc1C]

Present compound 10-1: ¹H-NMR (CDCl₃) δ: 7.67-7.62 (2H, m), 7.57 (1H, s), 7.36-7.28 (4H, m), 7.22 (1H, d), 7.16 (1H, s), 5.20 (2H, s), 3.79 (3H, s), 3.68 (3H, s), 2.24 (3H, s), 2.18 (3H, s).

### Preparation Example 7-1

The compounds which were prepared according to the Preparation Example 7 and their physical property value are shown below.

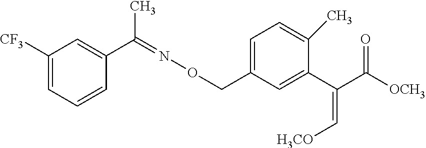 [SMILES: CO/C=C(/C(=O)OC)c1cc(CON=C(C)c2cccc(C(F)(F)F)c2)ccc1C]

Present compound 10-2: ¹H-NMR (CDCl₃) δ: 7.91 (1H, s), 7.83 (1H, d), 7.62-7.57 (2H, m), 7.47 (1H, t), 7.29 (1H, d), 7.23 (1H, d), 7.17 (1H, s), 5.22 (2H, s), 3.80 (3H, s), 3.69 (3H, s), 2.26 (3H, s), 2.19 (3H, s). CH₃ CH₃

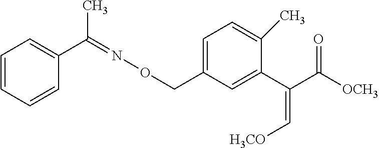 [SMILES: CO/C=C(/C(=O)OC)c1cc(CON=C(C)c2ccccc2)ccc1C]

Present compound 10-3: ¹H-NMR (CDCl₃) δ: 7.66-7.61 (2H, m), 7.39-7.33 (4H, m), 7.25 (1H, d), 7.16 (1H, d), 5.21 (2H, s), 4.03 (3H, s), 3.85 (3H, s), 2.25 (3H, s), 2.19 (3H, s).

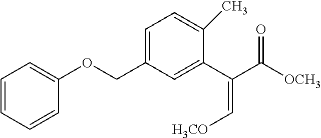 [SMILES: CO/C=C(/C(=O)OC)c1cc(COc2ccccc2)ccc1C]

Present compound 10-4: ¹H-NMR (CDCl₃) δ: 7.58 (1H, s), 7.33-7.16 (5H, m), 7.01-6.91 (3H, m), 5.02 (2H, s), 3.80 (3H, s), 3.70 (3H, s), 2.20 (3H, s).

### Preparation Example 7-2

The compound which can be prepared according to the Preparation Example 7 and their physical property value is shown below.

A compound represented by formula (2j):

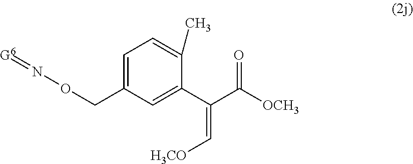

wherein G⁶ represents any substituents described in [Table A11].

TABLE A11

| Present | |
|---|---|
| Compound | G⁶ |
| 10-5 | 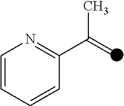 |
| 10-6 | 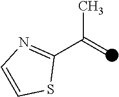 |
| 10-7 | 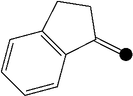 |
| 10-8 | 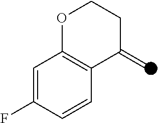 |
| 10-9 | 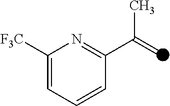 |
| 10-10 | 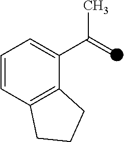 |
| 10-11 | 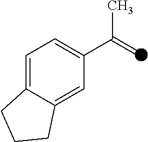 |
| 10-12 | 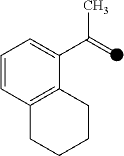 |

### Preparation Example 8

A mixture of 1-[3-(trifluoromethyl)phenyl]ethane-1-one hydrazone 0.24 g, the intermediate compound 15 0.30 g and ethanol 6 mL was stirred at 80° C. for 2 hours. The resulting mixture was cooled to room temperature, and water was added thereto, and the mixture was extracted with ethyl acetate. The resulting organic layers were washed with saturated brine, dried over anhydrous magnesium sulfate, and concentrated under reduced pressure. The resulting residue was subjected to a silica gel column chromatography (ethyl acetate:hexane=2:1) to obtain the present compound 11-1 represented by the following formula 0.21 g.

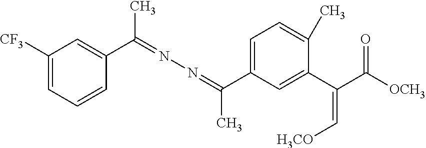 [SMILES: CO/C=C(/C(=O)OC)c1cc(C(C)=NN=C(C)c2cccc(C(F)(F)F)c2)ccc1C]

Present compound 11-1: ¹H-NMR (CDCl₃) δ: 8.15 (1H, s), 8.08 (1H, d), 7.79 (1H, dd), 7.67-7.65 (2H, m), 7.61 (1H, s), 7.54 (1H, t), 7.29 (1H, d), 3.85 (3H, s), 3.72 (3H, s), 2.32 (3H, s), 2.29 (3H, s), 2.23 (3H, s).

### Preparation Example 8-1

The compounds which were prepared according to the Preparation Example 8 and their physical property value are shown below.

A compound represented by formula (1k):

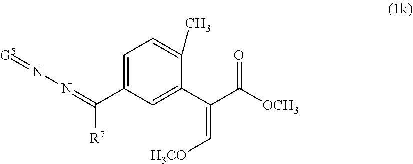

wherein a combination of G⁵ and R⁷ represents any combinations described in [Table A12].

TABLE A12

| Present | | |
|---|---|---|
| Compound | G⁵ | R⁷ |
| 11-2 | 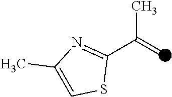 | Me |
| 11-3 | 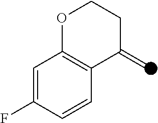 | Me |
| 11-16 | 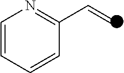 | Me |
| 11-17 | 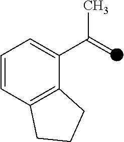 | Me |
| 11-18 | 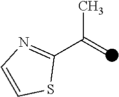 | Me |

Present compound 11-2: ¹H-NMR (CDCl₃) δ: 7.79 (1H, dd), 7.65 (1H, d), 7.60 (1H, s), 7.28 (1H, d), 6.94 (1H, d), 3.84 (3H, s), 3.71 (3H, s), 2.50 (3H, d), 2.43 (3H, s), 2.31 (3H, s), 2.23 (3H, s).

Present compound 11-3: ¹H-NMR (CDCl₃) δ: 8.20 (1H, dd), 7.77 (1H, dd), 7.63 (1H, d), 7.60 (1H, s), 7.28 (1H, s), 6.75-6.69 (1H, m), 6.65-6.61 (1H, m), 4.31 (2H, t), 3.84 (3H, s), 3.71 (3H, s), 3.00 (2H, t), 2.37 (3H, s), 2.22 (3H, s).

Present compound 11-6: LCMS: 352 [M+H]⁺, RT=1.80

[symbol]

Present compound 11-7: ¹H-NMR (CDCl₃) δ: 7.78 (1H, dd), 7.66 (1H, d), 7.60 (1H, s), 7.40 (1H, d), 7.29-7.27 (2H, m), 7.21 (1H, t), 3.84 (3H, s), 3.71 (3H, s), 3.23 (2H, t), 2.95 (2H, t), 2.30 (3H, s), 2.28 (3H, s), 2.22 (3H, s), 2.11-2.03 (2H, m).

Present compound 1 11-8: ¹H-NMR (CDCl₃) δ: 7.88 (1H, d), 7.80 (1H, dd), 7.66 (1H, d), 7.60 (1H, s), 7.39 (1H, d), 7.29 (1H, d), 3.84 (3H, s), 3.72 (3H, s), 2.46 (3H, s), 2.33 (3H, s), 2.23 (3H, s).

### Preparation Example 8-2

The compounds which can be prepared according to the Preparation example 8 are shown below.

The compounds represented by formula (1k) wherein a combination of G⁵ and R⁷ represents any combinations described in [Table A13].

TABLE A13

| Present | | |
|---|---|---|
| Compound | G⁵ | R⁷ |
| 11-4 | 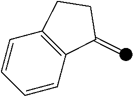 | Me |
| 11-6 | 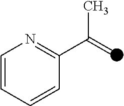 | H |
| 11-7 | 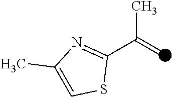 | H |
| 11-8 | 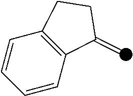 | H |
| 11-9 | 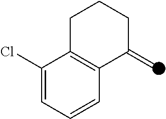 | Me |
| 11-10 | 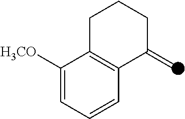 | Me |
| 11-11 | 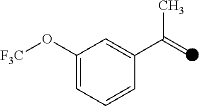 | Me |
| 11-12 | 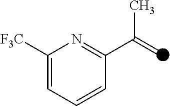 | Me |
| 11-13 | 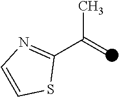 | Et |
| 11-14 | 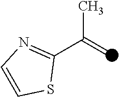 | Et |
| 11-15 | 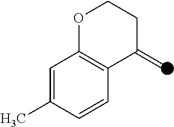 | Et |

### Preparation Example 8-3

The compounds which were prepared according to the Preparation Example 8 and their physical property value are shown below.

The compounds represented by formula (1k) wherein a combination of G⁵ and R⁷ represents any combinations described in [Table A13-2].

TABLE A13-2

| Present | | |
|---|---|---|
| Compound | G⁵ | R⁷ |
| 11-5 | 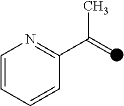 | Me |

Present compound 11-5: ¹H-NMR (CDCl₃) δ: 8.64 (1H, dq), 8.22 (1H, dt), 7.79 (1H, dd), 7.74 (1H, td), 7.66 (1H, d), 7.60 (1H, s), 7.31-7.28 (2H, m), 3.85 (3H, s), 3.72 (3H, s), 2.37 (3H, s), 2.25 (3H, s), 2.23 (3H, s).

### Preparation Example 8-4

The compounds which were prepared according to the Preparation Example 8 and their physical property value are shown below.

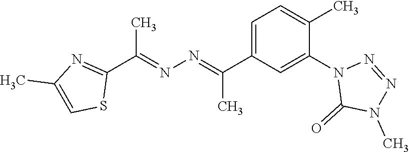 [SMILES: C/C(=N\N=C(/C)c1nc(C)cs1)c1ccc(C)c(-n2nnn(C)c2=O)c1]

Present compound 11-19: ¹H-NMR (CDCl₃) δ: 7.97 (1H, dd), 7.89 (1H, d), 7.42 (1H, d), 6.96 (1H, d), 3.74 (3H, s), 2.50 (3H, d), 2.45 (3H, s), 2.34 (6H, s).

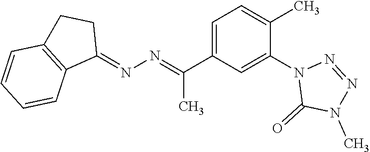 [SMILES: C/C(=N\N=C1/CCc2ccccc21)c1ccc(C)c(-n2nnn(C)c2=O)c1]

Present compound 11-20: ¹H-NMR (CDCl₃) δ: 7.97 (1H, dd), 7.92 (1H, d), 7.89 (1H, d), 7.44-7.30 (4H, m), 3.74 (3H, s), 3.12-3.06 (2H, m), 2.96-2.90 (2H, m), 2.41 (3H, s), 2.33 (3H, s).

### Preparation Example 9

A mixture of the present compound 2-9 0.23 g, 10% palladium/carbon 0.10 g, ammonium formate 0.06 g, and THE 6 mL was stirred at 80° C. for 5 hours. The resulting mixture was cooled to room temperature, and thereto was added aqueous saturated sodium hydrogencarbonate solution, and the mixture was filtered through Celite (registered trademark). The resulting filtrates were extracted with ethyl acetate, dried over anhydrous magnesium sulfate, and concentrated reduced pressure to obtain the present compound 12-1 represented by the following formula 0.21 g.

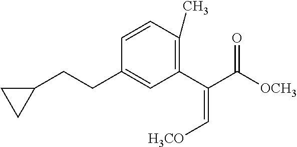 [SMILES: CO/C=C(/C(=O)OC)c1cc(CCC2CC2)ccc1C]

Present compound 12-1: ¹H-NMR (CDCl₃) δ: 7.55 (1H, s), 7.13 (1H, d), 7.04 (1H, dd), 6.92 (1H, d), 3.81 (3H, s), 3.70 (3H, s), 2.56 (2H, t), 2.14 (3H, s), 1.64-1.54 (2H, m), 1.34-1.30 (3H, m), 0.91-0.86 (2H, m).

### Preparation Example 9-1

The compounds which were prepared according to the Preparation Example 9 and their physical property value are shown below.

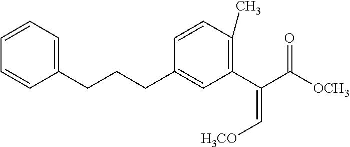 [SMILES: CO/C=C(/C(=O)OC)c1cc(CCCc2ccccc2)ccc1C]

Present compound 12-2: ¹H-NMR (CDCl₃) δ: 7.55 (1H, s), 7.30-7.25 (2H, m), 7.20-7.12 (4H, m), 7.05 (1H, dd), 6.93 (1H, d), 3.81 (3H, s), 3.69 (3H, s), 2.68-2.59 (4H, m), 2.14 (3H, s), 2.00-1.91 (2H, m).

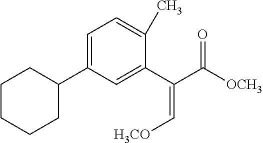 [SMILES: CO/C=C(/C(=O)OC)c1cc(C2CCCCC2)ccc1C]

Present compound 12-3: ¹H-NMR (CDCl₃) δ: 7.56 (1H, s), 7.14 (1H, d), 7.07 (1H, dd), 6.95 (1H, d), 3.82 (3H, s), 3.70 (3H, s), 2.51-2.41 (1H, m), 2.14 (3H, s), 1.92-1.69 (5H, m), 1.46-1.28 (5H, m).

### Preparation Example 10

A mixture of the intermediate compound 14 0.27 g, benzylbromide 0.15 mL, [1,1′-bisdiphenylphosphino)ferrocene]palladium (II) dichloride 0.03 g, tripotassium phosphate 0.52 g, and dimethoxyethane 5 mL was stirred under reflux for 5 hours under nitrogen atmosphere. The resulting mixture was filtered through Celite (registered trademark), and the filtrates were concentrated under reduced pressure. The resulting residue was subjected to a silica gel column chromatography (ethyl acetate:hexane=1:3) to obtain the present compound 3-1 represented by the following formula 0.09 g.

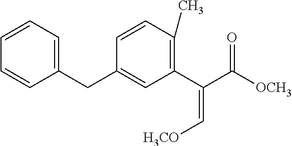 [SMILES: CO/C=C(/C(=O)OC)c1cc(Cc2ccccc2)ccc1C]

Present compound 13-1: ¹H-NMR (CDCl₃) δ: 7.53 (1H, d), 7.29-7.24 (2H, m), 7.21-7.11 (4H, m), 7.00 (1H, d), 6.95 (1H, s), 3.95 (2H, s), 3.79 (3H, s), 3.68 (3H, s), 2.14 (3H, s).

### Preparation Example 10-1

The compounds which were prepared according to the Preparation Example 10 and their physical property value are shown below.

A compound represented by formula (11):

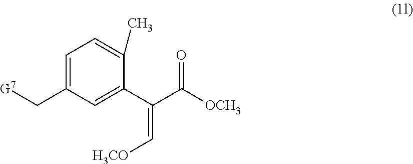

wherein G⁷ represents any substituents described in [Table A15].

TABLE A15

| Present | |
|---|---|
| Compound | G⁷ |
| 13-2 | 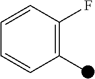 |
| 13-3 | 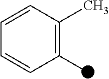 |
| 13-4 | 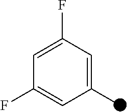 |

Present compound 13-2: ¹H-NMR (CDCl₃) δ: 7.54 (1H, s), 7.18-7.11 (3H, m), 7.06-6.96 (4H, m), 3.96 (2H, s), 3.79 (3H, s), 3.68 (3H, s), 2.14 (3H, s).

Present compound 13-3: ¹H-NMR (CDCl₃) δ: 7.52 (1H, s), 7.16-7.08 (5H, m), 6.95 (1H, dd), 6.89 (1H, d), 3.94 (2H, s), 3.80 (3H, s), 3.68 (3H, s), 2.25 (3H, s), 2.14 (3H, s).

Present compound 13-4: ¹H-NMR (CDCl₃) δ: 7.55 (1H, s), 7.16 (1H, d), 7.00 (1H, dd), 6.93 (1H, d), 6.74-6.67 (2H, m), 6.65-6.59 (1H, m), 3.91 (2H, s), 3.82 (3H, s), 3.70 (3H, s), 2.16 (3H, s).

### Preparation Example 12

A mixture of the intermediate compound 25 0.30 g, sodium hydride (60%, in oil) 0.05 g, and DMF 5 mL was stirred under ice-cooling for 15 minutes. To the resulting mixture was added 5-chloro-3-methyl-1,2,4-thiadiazole 0.22 g, and the mixture was stirred at room temperature for 5 hours. To the resulting mixture was added aqueous saturated ammonium chloride solution, and the mixture was extracted with MTBE. The resulting organic layers were washed with saturated brie, dried over anhydrous sodium sulfate, and concentrated under reduced pressure. The resulting residue was subjected to a silica gel column chromatography (ethyl acetate:hexane=1:4) to obtain the present compound 14-1 represented by the following formula 0.41 g.

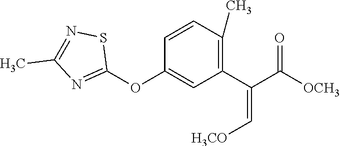 [SMILES: CO/C=C(/C(=O)OC)c1cc(Oc2nc(C)ns2)ccc1C]

Present compound 14-1: ¹H-NMR (CDCl₃) δ: 7.58 (1H, s), 7.29 (1H, d), 7.17 (1H, dd), 7.08 (1H, d), 3.85 (3H, s), 3.71 (3H, s), 2.51 (3H, s), 2.20 (3H, s).

### Preparation Example 12-1

The compounds which were prepared according to the Reference Preparation Example 3 and their physical property value are shown below.

A compound represented by formula (1m):

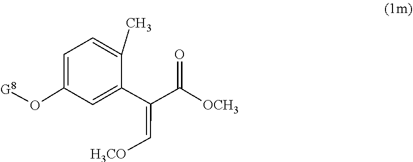

wherein G⁸ represents any substituents described in [Table A16].

TABLE A16

| Present | |
|---|---|
| Compound | G⁸ |
| 14-2 | 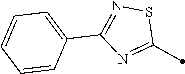 |
| 14-3 | 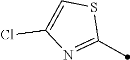 |

Present compound 14-2: ¹H-NMR (CDCl₃) δ: 8.22-8.19 (2H, m), 7.60 (1H, s), 7.47-7.42 (3H, m), 7.32 (1H, d), 7.23 (1H, dd), 7.16 (1H, d), 3.86 (3H, s), 3.73 (3H, s), 2.23 (3H, s).

Present compound 14-3: ¹H-NMR (CDCl₃) δ: 7.57 (1H, s), 7.26 (1H, d), 7.14 (1H, dd), 7.04 (1H, d), 6.54 (1H, s), 3.85 (3H, s), 3.71 (3H, s), 2.19 (3H, s).

A compound represented by formula (2m):

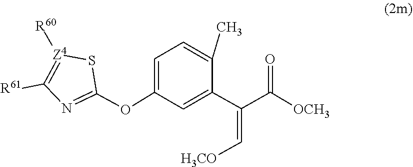

wherein a combination of R⁶⁰, R⁶¹, and Z⁴ represents any combinations described in [Table A16-2].

TABLE A16-2

| Present | | | |
|---|---|---|---|
| Compound | R⁶⁰ | R⁶¹ | Z⁴ |
| 14-4 | H | H | C |
| 14-5 | Me | H | C |
| 14-6 | Cl | H | C |
| 14-7 | Br | H | C |
| 14-8 | CF₃ | H | C |
| 14-9 | —C≡CH | H | C |
| 14-10 | —C≡C-c-Pr | H | C |
| 14-11 | —C≡C—Si(CH₃)₃ | H | C |
| 14-12 | c-Pr | H | C |
| 14-13 | H | Br | C |
| 14-14 | H | CF₃ | C |
| 14-15 | H | 3-Br—Ph | C |
| 14-16 | — | Cl | N |

Present compound 14-4: ¹H-NMR (CDCl₃) δ: 7.56 (1H, s), 7.27-7.21 (2H, m), 7.15 (1H, dd), 7.04 (1H, d), 6.78 (1H, d), 3.84 (3H, s), 3.70 (3H, s), 2.18 (3H, s).

Present compound 14-5: ¹H-NMR (CDCl₃) δ: 7.56 (1H, s), 7.23 (1H, d), 7.11 (1H, dd), 7.01 (1H, d), 6.87 (1H, s), 3.83 (3H, s), 3.70 (3H, s), 2.32 (3H, d), 2.17 (3H, s).

Present compound 14-6: ¹H-NMR (CDCl₃) δ: 7.57 (1H, s), 7.25 (1H, d), 7.12 (1H, dd), 7.05 (1H, s), 7.02 (1H, d), 3.84 (3H, s), 3.71 (3H, s), 2.18 (3H, s).

Present compound 14-7: ¹H-NMR (CDCl₃) δ: 7.57 (1H, s), 7.27-7.24 (1H, m), 7.16 (1H, s), 7.12 (1H, dd), 7.02 (1H, d), 3.84 (3H, s), 3.71 (3H, s), 2.18 (3H, s).

Present compound 14-8: ¹H-NMR (CDCl₃) δ: 7.58 (1H, s), 7.56 (1H, q), 7.29 (1H, d), 7.15 (1H, dd), 7.06 (1H, d), 3.85 (3H, s), 3.71 (3H, s), 2.20 (3H, s).

Present compound 14-9: ¹H-NMR (CDCl₃) δ: 7.57 (1H, s), 7.40 (1H, s), 7.26 (1H, d), 7.13 (1H, dd), 7.04 (1H, d), 3.84 (3H, s), 3.71 (3H, s), 3.31 (1H, s), 2.18 (3H, s).

Present compound 14-10: LCMS: 370 [M+H]⁺, RT=2.24 minutes Present compound 14-11: ¹H-NMR (CDCl₃) δ: 7.57 (1H, s), 7.35 (1H, s), 7.27-7.22 (1H, m), 7.12 (1H, dd), 7.02 (1H, d), 3.84 (3H, s), 3.71 (3H, s), 2.18 (3H, s), 0.22 (9H, s).

Present compound 14-12: ¹H-NMR (CDCl₃) δ: 7.56 (1H, s), 7.23 (1H, d), 7.11 (1H, dd), 7.00 (1H, d), 6.89 (1H, s), 3.83 (3H, s), 3.70 (3H, s), 2.17 (3H, s), 1.92-1.83 (1H, m), 0.95-0.87 (2H, m), 0.69-0.61 (2H, m).

Present compound 14-13: ¹H-NMR (CDCl₃) δ: 7.57 (1H, s), 7.26 (1H, d), 7.14 (1H, dd), 7.03 (1H, d), 6.69 (1H, s), 3.85 (3H, s), 3.71 (3H, s), 2.19 (3H, s).

Present compound 14-14: ¹H-NMR (CDCl₃) δ: 7.58 (1H, s), 7.28 (1H, d), 7.23-7.22 (1H, m), 7.17 (1H, dd), 7.07 (1H, d), 3.85 (3H, s), 3.71 (3H, s), 2.20 (3H, s).

Present compound 14-15: ¹H-NMR (CDCl₃) δ: 7.98 (1H, t), 7.76-7.72 (1H, m), 7.58 (1H, s), 7.44-7.40 (1H, m), 7.30-7.18 (3H, m), 7.12 (1H, d), 6.99 (1H, s), 3.85 (3H, s), 3.72 (3H, s), 2.20 (3H, s).

Present compound 14-16: ¹H-NMR (CDCl₃) δ: 7.59 (1H, s), 7.32 (1H, d), 7.17 (1H, dd), 7.08 (1H, d), 3.86 (3H, s), 3.72 (3H, s), 2.21 (3H, s).

A compound represented by formula (3m):

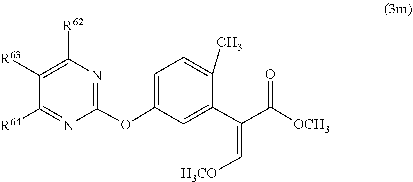

wherein a combination of R⁶², R⁶³ and R⁶⁴ represents any combinations described in [Table A16-3].

TABLE A16-3

| Present | | | |
|---|---|---|---|
| Compound | R⁶² | R⁶³ | R⁶⁴ |
| 14-17 | H | H | H |
| 14-18 | H | F | H |
| 14-19 | H | Cl | H |

Present compound 14-17: ¹H-NMR (CDCl₃) δ: 8.53 (2H, d), 7.57 (1H, s), 7.26 (1H, d), 7.09-7.04 (1H, m), 7.03-6.96 (2H, m), 3.82 (3H, s), 3.70 (3H, s), 2.19 (3H, s).

Present compound 14-18: ¹H-NMR (CDCl₃) δ: 8.39 (2H, s), 7.56 (1H, s), 7.26 (1H, d), 7.04 (1H, dd), 6.95 (1H, d), 3.82 (3H, s), 3.69 (3H, s), 2.19 (3H, s).

Present compound 14-19: ¹H-NMR (CDCl₃) δ: 8.37 (2H, s), 7.56 (1H, s), 7.26 (1H, d), 6.93 (1H, dd), 6.83 (1H, d), 3.85 (3H, s), 3.71 (3H, s), 2.18 (3H, s).

A compound represented by formula (4m):

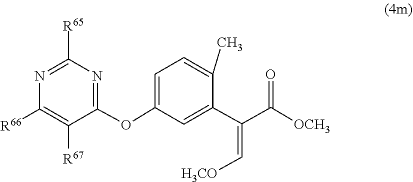

wherein a combination of R⁶⁵, R⁶⁶ and R⁶⁷ represents any combinations described in [Table A16-4].

TABLE A16-4

| Present | | | |
|---|---|---|---|
| Compound | R⁶⁵ | R⁶⁶ | R⁶⁷ |
| 14-20 | H | Cl | H |
| 14-21 | H | Cl | F |
| 14-22 | H | Me | H |
| 14-23 | H | Pho | H |
| 14-24 | H | 2-CN-PhO | H |
| 14-25 | H | 2,6-F₂-PhO | H |

Present compound 14-20: ¹H-NMR (CDCl₃) δ: 8.60 (1H, s), 7.58 (1H, s), 7.29 (1H, d), 7.01 (1H, dd), 6.91 (1H, d), 6.88 (1H, s), 3.85 (3H, s), 3.71 (3H, s), 2.20 (3H, s).

Present compound 14-21: ¹H-NMR (CDCl₃) δ: 8.30 (1H, s), 7.58 (1H, s), 7.29 (1H, d), 7.06 (1H, dd), 6.96 (1H, d), 3.84 (3H, s), 3.71 (3H, s), 2.20 (3H, s).

Present compound 14-22: ¹H-NMR (CDCl₃) δ: 8.68 (1H, s), 7.57 (1H, s), 7.27 (1H, d), 7.01 (1H, dd), 6.91 (1H, d), 6.69 (1H, s), 3.83 (3H, s), 3.71 (3H, s), 2.47 (3H, s), 2.20 (3H, s).

Present compound 14-23: ¹H-NMR (CDCl₃) δ: 8.45 (1H, d), 7.57 (1H, s), 7.47-7.41 (2H, m), 7.31-7.24 (2H, m), 7.17-7.12 (2H, m), 7.02 (1H, dd), 6.93 (1H, d), 6.29 (1H, d), 3.84 (3H, s), 3.71 (3H, s), 2.19 (3H, s).

Present compound 14-24: ¹H-NMR (CDCl₃) δ: 8.42 (1H, d), 7.72 (1H, dd), 7.69-7.64 (1H, m), 7.59 (1H, s), 7.37 (1H, td), 7.32-7.28 (2H, m), 7.05 (1H, dd), 6.96 (1H, d), 6.49 (1H, d), 3.85 (3H, s), 3.72 (3H, s), 2.21 (3H, s).

Present compound 14-25: ¹H-NMR (CDCl₃) δ: 8.41 (1H, d), 7.58 (1H, s), 7.29 (1H, d), 7.25-7.17 (1H, m), 7.07-6.99 (3H, m), 6.95 (1H, d), 6.47 (1H, d), 3.85 (3H, s), 3.72 (3H, s), 2.20 (3H, s).

A compound represented by formula (5m):

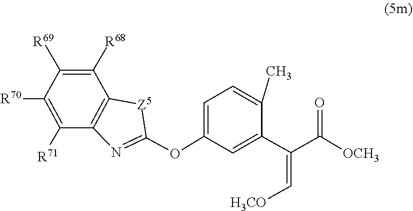

wherein a combination of R⁶⁸, R⁶⁹, R⁷⁰, R⁷, and Z⁵ represents any combinations described in [Table A16-5].

TABLE A16-5

| Present | | | | | |
|---|---|---|---|---|---|
| Compound | R⁶⁸ | R⁶⁹ | R⁷⁰ | R⁷¹ | Z⁵ |
| 14-26 | H | H | H | H | S |
| 14-27 | H | Cl | H | H | S |
| 14-28 | H | H | Cl | H | S |
| 14-29 | H | F | H | H | S |
| 14-30 | H | Me | H | H | S |
| 14-31 | H | H | OMe | H | S |
| 14-32 | H | F | H | F | S |
| 14-33 | H | Cl | H | H | O |

Present compound 14-26: ¹H-NMR (CDCl₃) δ: 7.75 (1H, d), 7.66 (1H, d), 7.58 (1H, s), 7.41-7.36 (1H, m), 7.31-7.20 (3H, m), 7.11 (1H, d), 3.85 (3H, s), 3.72 (3H, s), 2.21 (3H, s).

Present compound 14-27: ¹H-NMR (CDCl₃) δ: 7.64 (1H, d), 7.62 (1H, d), 7.58 (1H, s), 7.34 (1H, dd), 7.29 (1H, d), 7.22 (1H, dd), 7.11 (1H, d), 3.85 (3H, s), 3.72 (3H, s), 2.21 (3H, s).

Present compound 14-28: ¹H-NMR (CDCl₃) δ: 7.72 (1H, d), 7.58 (1H, s), 7.55 (1H, d), 7.29 (1H, d), 7.24-7.19 (2H, m), 7.11 (1H, d), 3.85 (3H, s), 3.72 (3H, s), 2.21 (3H, s).

Present compound 14-29: ¹H-NMR (CDCl₃) δ: 7.67 (1H, dd), 7.58 (1H, s), 7.35 (1H, dd), 7.29 (1H, d), 7.22 (1H, dd), 7.13-7.07 (2H, m), 3.84 (3H, s), 3.72 (3H, s), 2.21 (3H, s).

Present compound 14-30: ¹H-NMR (CDCl₃) δ: 7.62 (1H, d), 7.58 (1H, s), 7.44 (1H, s), 7.28 (1H, d), 7.24-7.17 (2H, m), 7.10 (1H, d), 3.84 (3H, s), 3.71 (3H, s), 2.42 (3H, s), 2.20 (3H, s).

Present compound 14-31: ¹H-NMR (CDCl₃) δ: 7.63 (1H, d), 7.57 (1H, s), 7.27 (1H, d), 7.22 (1H, dd), 7.15 (1H, d), 7.10 (1H, d), 6.97 (1H, dd), 3.84 (3H, s), 3.83 (3H, s), 3.71 (3H, s), 2.20 (3H, s).

Present compound 14-32: ¹H-NMR (CDCl₃) δ: 7.58 (1H, s), 7.29 (1H, d), 7.23 (1H, dd), 7.19-7.15 (1H, m), 7.13 (1H, d), 6.95-6.88 (1H, m), 3.86 (3H, s), 3.72 (3H, s), 2.21 (3H, s).

Present compound 14-33: ¹H-NMR (CDCl₃) δ: 7.58 (1H, s), 7.43-7.38 (2H, m), 7.29 (2H, d), 7.23 (1H, dd), 7.15 (1H, t), 3.84 (3H, s), 3.71 (3H, s), 2.19 (3H, s).

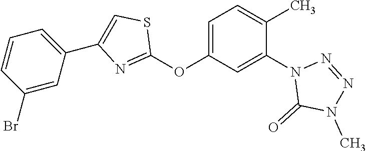 [SMILES: Cc1ccc(Oc2nc(-c3cccc(Br)c3)cs2)cc1-n1nnn(C)c1=O]

Present compound 14-34: ¹H-NMR (CDCl₃) δ: 7.95 (1H, t), 7.72 (1H, dt), 7.46-7.40 (4H, m), 7.28-7.22 (1H, m), 7.05 (1H, s), 3.73 (3H, s), 2.34 (3H, s).

A compound represented by formula (6m):

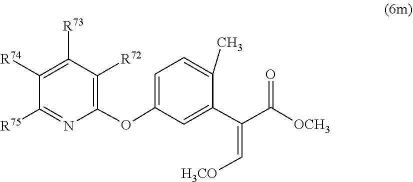

wherein a combination of R⁷², R⁷³, R⁷⁴, and R⁷⁵ represents any combinations described in [Table A16-6].

TABLE A16-6

| Present | | | | |
|---|---|---|---|---|
| Compound | R⁷² | R⁷³ | R⁷⁴ | R⁷⁵ |
| 4-59 | Cl | H | H | H |
| 4-60 | H | H | Cl | H |
| 4-61 | H | H | H | Cl |
| 4-62 | H | H | F | H |
| 4-63 | H | H | CF₃ | H |
| 4-64 | Cl | H | Cl | H |
| 4-65 | F | H | F | H |
| 4-66 | Cl | H | Cl | Cl |

Present compound 4-59: ¹H-NMR (CDCl₃) δ: 8.12 (1H, dd), 7.55 (1H, s), 7.25-7.20 (2H, m), 7.16 (1H, dd), 6.89 (1H, dd), 6.81 (1H, d), 3.84 (3H, s), 3.71 (3H, s), 2.17 (3H, s).

Present compound 4-60: ¹H-NMR (CDCl₃) δ: 8.13 (1H, d), 7.60 (1H, dd), 7.56 (1H, s), 7.24 (1H, d), 6.99 (1H, dd), 6.89 (1H, d), 6.84 (1H, d), 3.83 (3H, s), 3.70 (3H, s), 2.18 (3H, s).

Present compound 4-61: ¹H-NMR (CDCl₃) δ: 7.60-7.53 (2H, m), 7.23 (1H, d), 7.03-6.97 (2H, m), 6.92 (1H, d), 6.71 (1H, d), 3.82 (3H, s), 3.70 (3H, s), 2.18 (3H, s).

Present compound 4-62: ¹H-NMR (CDCl₃) δ: 8.03 (1H, d), 7.55 (1H, s), 7.42-7.36 (1H, m), 7.23 (1H, d), 6.98 (1H, dd), 6.89 (1H, d), 6.86 (1H, dd), 3.82 (3H, s), 3.70 (3H, s), 2.17 (3H, s).

Present compound 4-63: ¹H-NMR (CDCl₃) δ: 8.46 (1H, s), 7.86 (1H, dd), 7.57 (1H, s), 7.27 (1H, d), 7.02 (1H, dd), 6.97 (1H, d), 6.92 (1H, d), 3.84 (3H, s), 3.71 (3H, s), 2.19 (3H, s).

Present compound 4-64: ¹H-NMR (CDCl₃) δ: 7.96 (1H, d), 7.74 (1H, d), 7.56 (1H, s), 7.25 (1H, d), 7.01 (1H, dd), 6.91 (1H, d), 3.83 (3H, s), 3.70 (3H, s), 2.18 (3H, s).

Present compound 4-65: ¹H-NMR (CDCl₃) δ: 7.71 (1H, t), 7.56 (1H, s), 7.24 (1H, d), 7.09 (1H, td), 6.92 (1H, t), 6.85 (1H, d), 3.85 (3H, s), 3.71 (3H, s), 2.19 (3H, s).

Present compound 4-66: ¹H-NMR (CDCl₃) δ: 7.81 (1H, s), 7.57 (1H, s), 7.24 (1H, d), 7.04 (1H, dd), 6.93 (1H, d), 3.85 (3H, s), 3.71 (3H, s), 2.19 (3H, s).

A compound represented by formula (7m):

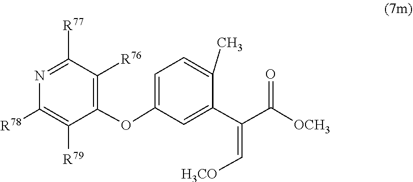

wherein a combination of R⁷⁶, R⁷⁷, R⁷⁸, and R⁷⁹ represents any combinations described in [Table A16-7].

TABLE A16-7

| Present | | | | |
|---|---|---|---|---|
| Compound | R⁷⁶ | R⁷⁷ | R⁷⁸ | R⁷⁹ |
| 4-67 | Cl | H | H | H |
| 4-68 | H | F | H | H |

Present compound 4-67: ¹H-NMR (CDCl₃) δ: 8.55 (1H, s), 8.27 (1H, d), 7.57 (1H, s), 7.28 (1H, d), 6.97 (1H, dd), 6.89 (1H, d), 6.71 (1H, d), 3.85 (3H, s), 3.72 (3H, s), 2.20 (3H, s).

Present compound 4-68: ¹H-NMR (CDCl₃) δ: 8.05 (1H, d), 7.57 (1H, s), 7.28 (1H, d), 6.96 (1H, dd), 6.88 (1H, d), 6.78 (1H, dd), 6.41 (1H, d), 3.86 (3H, s), 3.72 (3H, s), 2.20 (3H, s).

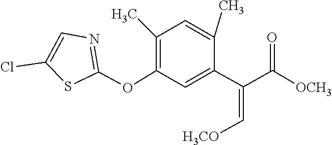 [SMILES: CO/C=C(/C(=O)OC)c1cc(Oc2ncc(Cl)s2)c(C)cc1C]

Present compound 14-44: ¹H-NMR (CDCl₃) δ: 7.56 (1H, s), 7.13 (1H, s), 7.03 (1H, s), 6.96 (1H, s), 3.84 (3H, s), 3.70 (3H, s), 2.23 (3H, s), 2.16 (3H, s).

### Preparation Example 12-2

The compounds which can be prepared according to the Preparation Example 12 are shown below.

A compound represented by formula (8m):

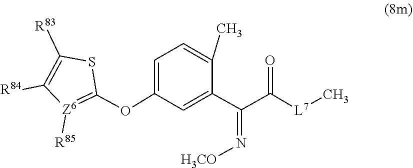

wherein a combination of R⁸³, R⁸⁴, R⁸⁵, Z⁶ and L⁷ represents any combinations described in [Table A16-8].

TABLE A16-8

| Present | | | | | |
|---|---|---|---|---|---|
| Compound | R⁸³ | R⁸⁴ | R⁸⁵ | Z⁶ | L⁷ |
| 14-35 | H | H | H | C | O |
| 14-36 | H | H | — | N | O |
| 14-37 | Cl | H | — | N | O |
| 14-38 | Br | H | — | N | O |
| 14-39 | CF₃ | H | — | N | O |
| 14-40 | —C≡CH | H | — | N | O |
| 14-41 | —C≡C-c-Pr | H | — | N | O |
| 14-42 | c-Pr | H | — | N | O |
| 14-43 | H | CF₃ | — | N | O |
| 14-45 | H | H | H | C | NH |
| 14-46 | H | H | — | N | NH |
| 14-47 | Cl | H | — | N | NH |
| 14-48 | Br | H | — | N | NH |
| 14-49 | CF₃ | H | — | N | NH |
| 14-50 | —C≡CH | H | — | N | NH |
| 14-51 | —C≡C-c-Pr | H | — | N | NH |
| 14-52 | c-Pr | H | — | N | NH |
| 14-53 | H | CF₃ | — | N | NH |

### Preparation Example 13

At room temperature, to a mixture of 1-bromo-3-(trifluoromethoxy)benzene 0.20 g, copper (I) iodide 0.14 g, N,N′-dimethylcyclohexane-1,2-diamine 0.14 mL, potassium carbonate 0.19 g, and DMF 4 mL was added the intermediate compound 25 0.15 g, and the mixture was stirred at 90° C. for 6 hours. Water was added to the resulting mixture, and the mixture was extracted with MTBE. The resulting organic layers were washed with saturated brine, dried over anhydrous sodium sulfate, and concentrated under reduced pressure. The resulting residue was subjected to a silica gel column chromatography (ethyl acetate:hexane=1:3) to obtain the present compound 4-17 represented by the following formula 0.19 g.

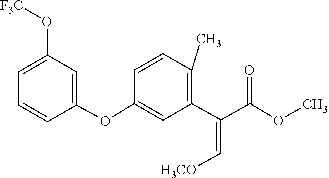 [SMILES: CO/C=C(/C(=O)OC)c1cc(Oc2cccc(OC(F)(F)F)c2)ccc1C]

Present compound 4-17: ¹H-NMR (CDCl₃) δ: 7.55 (1H, s), 7.29 (1H, t), 7.21 (1H, d), 6.96-6.88 (3H, m), 6.86 (1H, s), 6.83 (1H, d), 3.83 (3H, s), 3.70 (3H, s), 2.17 (3H, s).

### Preparation Example 13-1

The compounds which were prepared according to the Preparation Example 13 and their physical property value are shown below.

The compounds represented by formula (1d) wherein a combination of R²⁹, R³⁰, R³¹, R³², and R³³ represents any combinations described in [Table A17].

TABLE A17

| Present | | | | | |
|---|---|---|---|---|---|
| Compound | R²⁹ | R³⁰ | R³¹ | R³² | R³³ |
| 4-27 | H | F | H | F | H |
| 4-34 | H | t-Bu | H | H | H |
| 4-36 | H | OPh | H | H | H |
| 4-21 | Cl | H | H | H | H |
| 4-22 | H | H | Cl | H | H |
| 4-24 | F | H | F | H | H |
| 4-28 | OMe | H | H | H | H |
| 4-29 | H | H | OMe | H | H |

Present compound 4-27: ¹H-NMR (CDCl₃) δ: 7.56 (1H, s), 7.23 (1H, d), 6.91 (1H, dd), 6.84 (1H, d), 6.52-6.47 (2H, m), 6.38-6.33 (1H, m), 3.84 (3H, s), 3.72 (3H, s), 2.18 (3H, s).

Present compound 4-34: ¹H-NMR (CDCl₃) δ: 7.53 (1H, s), 7.23 (1H, t), 7.17 (1H, d), 7.12-7.08 (2H, m), 6.87 (1H, dd), 6.83-6.78 (2H, m), 3.82 (3H, s), 3.69 (3H, s), 2.15 (3H, s), 1.30 (9H, s).

Present compound 4-36: ¹H-NMR (CDCl₃) δ: 7.53 (1H, s), 7.35-7.28 (2H, m), 7.25-7.19 (1H, m), 7.17 (1H, d), 7.12-7.06 (1H, m), 7.03-6.99 (2H, m), 6.89 (1H, dd), 6.83 (1H, d), 6.75-6.65 (3H, m), 3.79 (3H, s), 3.68 (3H, s), 2.15 (3H, s).

Present compound 4-21: ¹H-NMR (CDCl₃) δ: 7.54 (1H, s), 7.43 (1H, dd), 7.21-7.15 (2H, m), 7.03 (1H, td), 6.98 (1H, dd), 6.84 (1H, dd), 6.79 (1H, d), 3.83 (3H, s), 3.70 (3H, s), 2.15 (3H, s).

Present compound 4-22: ¹H-NMR (CDCl₃) δ: 7.54 (1H, s), 7.28-7.23 (2H, m), 7.18 (1H, d), 6.97-6.92 (2H, m), 6.86 (1H, dd), 6.79 (1H, d), 3.83 (3H, s), 3.70 (3H, s), 2.16 (3H, s).

Present compound 4-24: ¹H-NMR (CDCl₃) δ: 7.54 (1H, s), 7.15 (1H, d), 7.08-7.00 (1H, m), 6.96-6.89 (1H, m), 6.85-6.77 (2H, m), 6.75 (1H, d), 3.83 (3H, s), 3.70 (3H, s), 2.14 (3H, s).

Present compound 4-28: ¹H-NMR (CDCl₃) δ: 7.53 (1H, s), 7.13 (1H, d), 7.11-7.06 (1H, m), 7.00-6.95 (2H, m), 6.91-6.86 (1H, m), 6.82-6.77 (2H, m), 3.84 (3H, s), 3.81 (3H, s), 3.68 (3H, s), 2.13 (3H, s).

Present compound 4-29: ¹H-NMR (CDCl₃) δ: 7.53 (1H, s), 7.13 (1H, d), 7.01-6.95 (2H, m), 6.89-6.83 (2H, m), 6.79 (1H, dd), 6.75 (1H, d), 3.82 (3H, s), 3.80 (3H, s), 3.70 (3H, s), 2.13 (3H, s).

A compound represented by formula (2p):

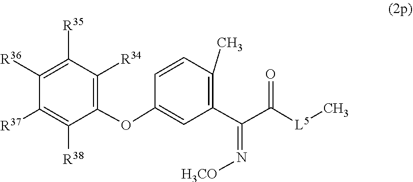

wherein a combination of L⁵, R³⁴, R³⁵, R³⁶, R³⁷, and R³⁸ represents any combinations described in [Table A18].

TABLE A18

| Present | | | | | | |
|---|---|---|---|---|---|---|
| Compound | L⁵ | R³⁴ | R³⁵ | R³⁶ | R³⁷ | R³⁸ |
| 4-37 | O | H | F | H | H | H |
| 4-38 | NH | H | F | H | H | H |
| 4-39 | O | Me | H | H | H | H |
| 4-40 | NH | Me | H | H | H | H |

Present compound 4-37: ¹H-NMR (CDCl₃) δ: 7.32-7.22 (4H, m), 7.16 (1H, dd), 6.98 (1H, dd), 6.82 (1H, d), 4.06 (3H, s), 3.87 (3H, s), 2.17 (3H, s).

Present compound 4-38: ¹H-NMR (CDCl₃) δ: 7.28-7.19 (2H, m), 6.96 (1H, dd), 6.81-6.70 (5H, m), 3.97 (3H, s), 2.93 (3H, d), 2.16 (3H, s).

Present compound 4-39: ¹H-NMR (CDCl₃) δ: 7.25-7.21 (1H, m), 7.17-7.12 (2H, m), 7.06-7.02 (1H, m), 6.90 (1H, dd), 6.85 (1H, dd), 6.69 (1H, d), 4.04 (3H, s), 3.86 (3H, s), 2.24 (3H, s), 2.14 (3H, s).

Present compound 4-40: ¹H-NMR (CDCl₃) δ: 7.22 (1H, d), 7.16-7.10 (2H, m), 7.06-7.00 (1H, m), 6.92 (1H, d), 6.82 (1H, dd), 6.75 (1H, br s), 6.69 (1H, d), 3.95 (3H, s), 2.90 (3H, d), 2.24 (3H, s), 2.13 (3H, s).

A compound represented by formula (20):

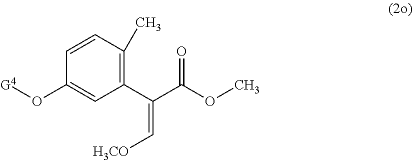

wherein G⁴ represents any substituents described in [Table A19] and [Tale 19-2].

TABLE A19

| Present | |
|---|---|
| Compound | G⁴ |
| 4-41 | 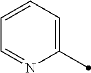 |
| 4-42 | 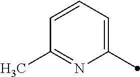 |
| 4-43 | 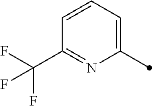 |
| 4-44 | 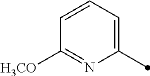 |
| 4-45 | 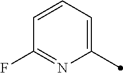 |
| 4-46 | 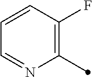 |

TABLE A19-2

| Present | |
|---|---|
| Compound | G⁴ |
| 4-69 | 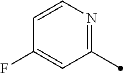 |
| 4-70 | 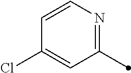 |
| 4-71 | 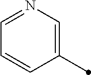 |
| 4-72 | 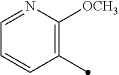 |
| 4-73 | 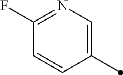 |
| 4-74 | 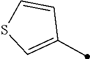 |
| 4-75 | 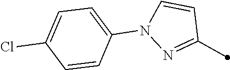 |
| 4-76 | 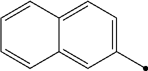 |
| 4-77 | 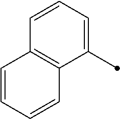 |

Present compound 4-41: ¹H-NMR (CDCl₃) δ: 8.21-8.19 (1H, m), 7.64 (1H, ddd), 7.55 (1H, s), 7.23 (1H, d), 7.01 (1H, dd), 6.96 (1H, ddd), 6.91 (1H, d), 6.86 (1H, dt), 3.82 (3H, s), 3.70 (3H, s), 2.17 (3H, s).

Present compound 4-42: ¹H-NMR (CDCl₃) δ: 7.55 (1H, s), 7.52-7.48 (1H, m), 7.21 (1H, d), 6.99 (1H, dd), 6.91 (1H, d), 6.84 (1H, d), 6.55 (1H, d), 3.83 (3H, s), 3.70 (3H, s), 2.47 (3H, s), 2.17 (3H, s).

Present compound 4-43: ¹H-NMR (CDCl₃) δ: 7.79 (1H, t), 7.56 (1H, s), 7.36 (1H, d), 7.24 (1H, s), 7.05 (1H, dd), 7.00 (1H, d), 6.95 (1H, d), 3.83 (3H, s), 3.70 (3H, s), 2.19 (3H, s).

Present compound 4-44: ¹H-NMR (CDCl₃) δ: 7.55 (1H, s), 7.50 (1H, t), 7.22 (1H, d), 7.02 (1H, dd), 6.93 (1H, d), 6.42 (1H, d), 6.30 (1H, d), 3.84 (3H, s), 3.82 (3H, s), 3.70 (3H, s), 2.18 (3H, s).

Present compound 4-45: ¹H-NMR (CDCl₃) δ: 7.71 (1H, q), 7.56 (1H, s), 7.24 (1H, d), 7.01 (1H, dd), 6.92 (1H, d), 6.67 (1H, dd), 6.58 (1H, dd), 3.84 (3H, s), 3.71 (3H, s), 2.18 (3H, s).

Present compound 4-46: ¹H-NMR (CDCl₃) δ: 7.92 (1H, d), 7.56 (1H, s), 7.44 (1H, t), 7.24 (1H, d), 7.04 (1H, d), 6.98-6.94 (2H, m), 3.83 (3H, s), 3.70 (3H, s), 2.18 (3H, s).

Present compound 4-69: ¹H-NMR (CDCl₃) δ: 8.16 (1H, dd), 7.56 (1H, s), 7.25 (1H, d), 7.01 (1H, dd), 6.91 (1H, d), 6.75-6.71 (1H, m), 6.55 (1H, dd), 3.83 (3H, s), 3.70 (3H, s), 2.18 (3H, s).

Present compound 4-70: ¹H-NMR (CDCl₃) δ: 8.10 (1H, d), 7.56 (1H, s), 7.25 (1H, d), 7.02-6.96 (2H, m), 6.90 (1H, d), 6.88 (1H, d), 3.83 (3H, s), 3.71 (3H, s), 2.18 (3H, s).

Present compound 4-71: ¹H-NMR (CDCl₃) δ: 8.41 (1H, d), 8.33 (1H, dd), 7.55 (1H, s), 7.32-7.28 (1H, m), 7.26-7.19 (2H, m), 6.89 (1H, dd), 6.82 (1H, d), 3.84 (3H, s), 3.71 (3H, s), 2.17 (3H, s).

Present compound 4-72: ¹H-NMR (CDCl₃) δ: 7.89 (1H, dd), 7.54 (1H, s), 7.19-7.14 (2H, m), 6.86-6.78 (3H, m), 4.01 (3H, s), 3.82 (3H, s), 3.69 (3H, s), 2.15 (3H, s).

Present compound 4-73: ¹H-NMR (CDCl₃) δ: 7.97-7.95 (1H, m), 7.55 (1H, s), 7.47-7.41 (1H, m), 7.20 (1H, d), 6.92-6.84 (2H, m), 6.79 (1H, d), 3.84 (3H, s), 3.71 (3H, s), 2.16 (3H, s).

Present compound 4-74: ¹H-NMR (CDCl₃) δ: 7.54 (1H, s), 7.22 (1H, dd), 7.16 (1H, d), 6.91 (1H, dd), 6.88-6.85 (2H, m), 6.57 (1H, dd), 3.83 (3H, s), 3.70 (3H, s), 2.14 (3H, s).

Present compound 4-75: ¹H-NMR (CDCl₃) δ: 7.76 (1H, d), 7.60-7.54 (3H, m), 7.40-7.35 (2H, m), 7.19 (1H, d), 7.06 (1H, dd), 6.98 (1H, d), 5.99 (1H, d), 3.83 (3H, s), 3.70 (3H, s), 2.16 (3H, s).

Present compound 4-76: ¹H-NMR (CDCl₃) δ: 7.81 (2H, d), 7.69 (1H, d), 7.54 (1H, s), 7.46-7.35 (2H, m), 7.32 (1H, d), 7.28 (1H, dd), 7.21 (1H, d), 6.94 (1H, dd), 6.88 (1H, d), 3.83 (3H, s), 3.71 (3H, s), 2.18 (3H, s).

Present compound 4-77: ¹H-NMR (CDCl₃) δ: 8.26 (1H, dd), 7.86 (1H, dd), 7.57 (1H, d), 7.55-7.45 (3H, m), 7.35 (1H, t), 7.19 (1H, d), 6.94 (1H, dd), 6.91 (1H, dd), 6.88 (1H, d), 3.82 (3H, s), 3.70 (3H, s), 2.17 (3H, s).

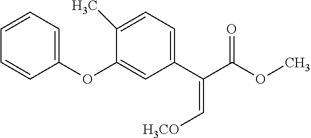 [SMILES: CO/C=C(/C(=O)OC)c1ccc(C)c(Oc2ccccc2)c1]

Present compound 4-47: ¹H-NMR (CDCl₃) δ: 7.50 (1H, s), 7.32-7.26 (2H, m), 7.23 (1H, d), 7.08-6.99 (2H, m), 6.96-6.92 (3H, m), 3.82 (3H, s), 3.71 (3H, s), 2.22 (3H, s).

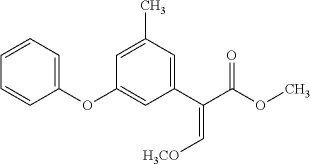 [SMILES: CO/C=C(/C(=O)OC)c1cc(C)cc(Oc2ccccc2)c1]

Present compound 4-48: ¹H-NMR (CDCl₃) δ: 7.52 (1H, s), 7.36-7.29 (2H, m), 7.11-7.00 (3H, m), 6.90 (1H, s), 6.82 (1H, s), 6.74-6.72 (1H, m), 3.84 (3H, s), 3.73 (3H, s), 2.32 (3H, s).

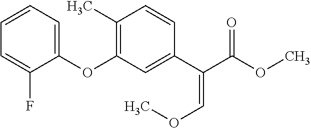 [SMILES: CO/C=C(/C(=O)OC)c1ccc(C)c(Oc2ccccc2F)c1]

Present compound 4-78: ¹H-NMR (CDCl₃) δ: 7.49 (1H, s), 7.22 (1H, d), 7.19-7.13 (1H, m), 7.06 (1H, dd), 7.04-7.00 (2H, m), 6.92-6.86 (1H, m), 6.85 (1H, d), 3.82 (3H, s), 3.70 (3H, s), 2.28 (3H, s).

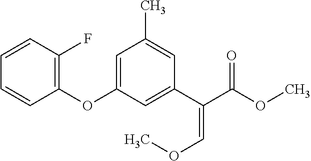 [SMILES: CO/C=C(/C(=O)OC)c1cc(C)cc(Oc2ccccc2F)c1]

Present compound 4-79: ¹H-NMR (CDCl₃) δ: 7.52 (1H, s), 7.20-7.03 (4H, m), 6.89 (1H, s), 6.80 (1H, s), 6.70 (1H, s), 3.84 (3H, s), 3.72 (3H, s), 2.32 (3H, s).

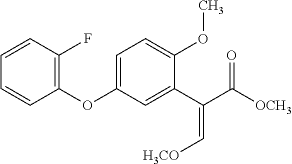 [SMILES: CO/C=C(/C(=O)OC)c1cc(Oc2ccccc2F)ccc1OC]

Present compound 4-80: ¹H-NMR (CDCl₃) δ: 7.48 (1H, s), 7.19-7.12 (1H, m), 7.06-6.98 (2H, m), 6.96-6.89 (2H, m), 6.86 (1H, d), 6.82-6.74 (1H, m), 3.81 (3H, s), 3.77 (3H, s), 3.70 (3H, s).

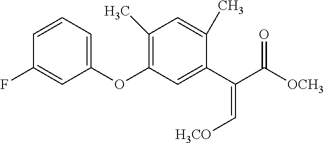 [SMILES: CO/C=C(/C(=O)OC)c1cc(Oc2cccc(F)c2)c(C)cc1C]

Present compound 4-81: ¹H-NMR (CDCl₃) δ: 7.53 (1H, s), 7.25-7.17 (1H, m), 7.12 (1H, s), 6.75 (1H, s), 6.73-6.67 (2H, m), 6.61 (1H, dt), 3.83 (3H, s), 3.69 (3H, s), 2.16 (3H, s), 2.15 (3H, s).

### Preparation Example 13-2

The compounds which can be prepared according to the Preparation Exmaple 13 are shown below.

A compound represented by formula (2p) wheein L⁵, R³⁴, R³⁵, R³⁶, R³⁷, and R³⁸ represents any combinations described in [Table A20].

TABLE A20

| Present | | | | | | |
|---|---|---|---|---|---|---|
| Compound | L⁵ | R³⁴ | R³⁵ | R³⁶ | R³⁷ | R³⁸ |
| 4-49 | O | H | Me | H | H | H |
| 4-50 | O | H | H | Me | H | H |
| 4-51 | O | H | OMe | H | H | H |
| 4-52 | O | H | OCF₃ | H | H | H |
| 4-53 | O | H | OPh | H | H | H |
| 4-54 | NH | H | Me | H | H | H |
| 4-55 | NH | H | H | Me | H | H |
| 4-56 | NH | H | OMe | H | H | H |
| 4-57 | NH | H | OCF₃ | H | H | H |
| 4-58 | NH | H | OPh | H | H | H |

### Preparation Example 1A

A mixture of the intermediate compound 25 0.20 g, benzylbromide 0.14 mL, potassium carbonate 0.25 g, and DMF 3 mL was stirred at room temperature overnight. Water was added to the resulting mixture, and the mixture was extracted with MTBE. The resulting organic layers were washed with saturated brine, dried over anhydrous sodium sulfate, and concentrated under reduced pressure. The resulting residue was subjected to a silica gel column chromatography (ethyl acetate:hexane=1:4) to obtain the present compound 1A-1 represented by the following formula 0.2 g.

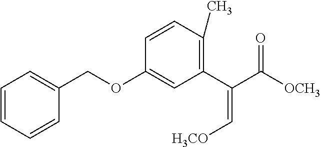 [SMILES: CO/C=C(/C(=O)OC)c1cc(OCc2ccccc2)ccc1C]

Present compound 1A-1: ¹H-NMR (CDCl₃) δ: 7.54 (1H, s), 7.45-7.40 (2H, m), 7.40-7.34 (2H, m), 7.34-7.28 (1H, m), 7.13 (1H, d), 6.86 (1H, dd), 6.76 (1H, d), 5.02 (2H, s), 3.81 (3H, s), 3.69 (3H, s), 2.11 (3H, s).

### Preparation Example 1A-1

The compounds which were prepared according to the Preparation Example 1A and their physical property value are shown below.

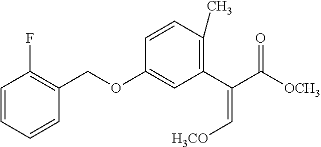 [SMILES: CO/C=C(/C(=O)OC)c1cc(OCc2ccccc2F)ccc1C]

Present compound 1A-2: ¹H-NMR (CDCl₃) δ: 7.55 (1H, s), 7.52 (1H, td), 7.33-7.26 (1H, m), 7.18-7.12 (2H, m), 7.09-7.04 (1H, m), 6.87 (1H, dd), 6.77 (1H, d), 5.10 (2H, s), 3.82 (3H, s), 3.70 (3H, s), 2.11 (3H, s).

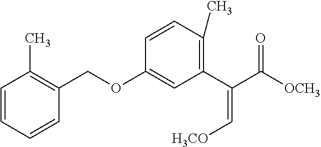 [SMILES: CO/C=C(/C(=O)OC)c1cc(OCc2ccccc2C)ccc1C]

Present compound 1A-3: ¹H-NMR (CDCl₃) δ: 7.55 (1H, s), 7.43-7.39 (1H, m), 7.24-7.17 (3H, m), 7.14 (1H, d), 6.87 (1H, dd), 6.77 (1H, d), 4.99 (2H, s), 3.82 (3H, s), 3.70 (3H, s), 2.37 (3H, s), 2.12 (3H, s).

### Preparation Example 2A

To a mixture of sodium hydride (60%, in oil) 0.04 g, and DNF 2 mL was added 1-(4-chlorophenyl)-1H-pyrazole-3-ol 0.18 g at 0° C., and the mixture was stirred at 0° C. for 30 minutes. To the resulting mixture were added a mixture of methyl (E)-2-(5-bromomethyl-2-methylphenyl)-3-methoxyacryate (which was prepared by a method described in WO 1999/028305 A1) 0.25 g and DMF 2 mL, and the mixture was stirred at room temperature for 3.5 hours. To the resulting mixture was added aqueous ammonium chloride solution, and the mixture was extracted with ethyl acetate. The resulting organic layers were washed with water and saturated brine successively, dried over sodium sulfate, and concentrated under reduced pressure. The resulting residue was was subjected to a silica gel column chromatography (hexane: ethyl acetate=1:3) to obtain the present compound 2A-1 represented by the following formula 0.18 g.

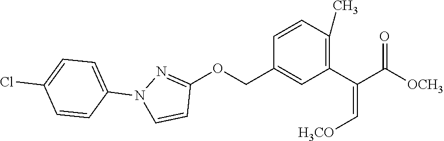 [SMILES: CO/C=C(/C(=O)OC)c1cc(COc2ccn(-c3ccc(Cl)cc3)n2)ccc1C]

Present compound 2A-1: ¹H-NMR (CDCl₃) δ: 7.69 (1H, d), 7.58-7.53 (3H, m), 7.39-7.32 (3H, m), 7.26-7.22 (2H, m), 5.92 (1H, d), 5.26 (2H, s), 3.81 (3H, s), 3.69 (3H, s), 2.19 (3H, s).

### Preparation Example 2A-1

The compounds which were prepared according to the Preparation Example 2A and their physical property value are shown below.

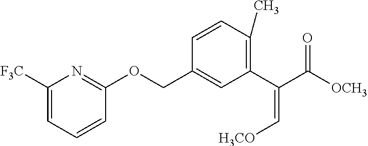 [SMILES: CO/C=C(/C(=O)OC)c1cc(COc2cccc(C(F)(F)F)n2)ccc1C]

Present compound 2A-2: ¹H-NMR (CDCl₃) δ: 7.68 (1H, t), 7.57 (1H, s), 7.35 (1H, dd), 7.26-7.22 (3H, m), 6.93 (1H, d), 5.38 (2H, s), 3.81 (3H, s), 3.69 (3H, s), 2.18 (3H, s).

A compound represented by formula (1n):

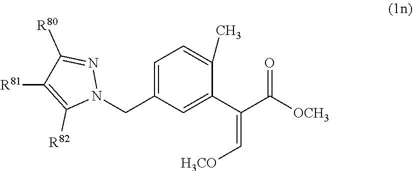

wherein a combination of R⁸⁰, R⁸¹, and R⁸² represents any combinations described in [Table A21].

TABLE A21

| Present | | | |
|---|---|---|---|
| Compound | R⁸⁰ | R⁸¹ | R⁸² |
| 2A-3 | i-Pr | H | H |
| 2A-4 | H | Cl | H |

Present compound 2A-3: ¹H-NMR (CDCl₃) δ: 7.55 (1H, s), 7.21 (1H, d), 7.18 (1H, d), 7.04 (1H, dd), 6.99 (1H, d), 6.05 (1H, d), 5.22 (2H, s), 3.81 (3H, s), 3.69 (3H, s), 3.05-2.97 (1H, m), 2.16 (3H, s), 1.30-1.25 (6H, m).

Present compound 2A-4: ¹H-NMR (CDCl₃) δ: 7.56 (1H, s), 7.44 (1H, s), 7.32 (1H, s), 7.21 (1H, d), 7.07 (1H, dd), 7.00 (1H, d), 5.21 (2H, s), 3.82 (3H, s), 3.70 (3H, s), 2.17 (3H, s).

### Preparation Example 3A

Under argon atmosphere, a mixture of the intermediate compound represented 25 0.30 g, 4-chlorothiophenol 0.20 g, tris(dibenzylideneacetone)dipalladium (0) 0.05 g, 1,1′-bis(diphenylphosphino)ferrocene 0.06 g, and diisopropylethylamine 0.36 mL, and DMF 5 mL was stirred at 110° C. for 7 hours. Water was added to the resulting mixture, and the mixture was extracted with ethyl acetate. The resulting organic layers were washed with saturated brine, dried over anhydrous sodium sulfate, and concentrated under reduced pressure. The resulting residue was subjected to a silica gel column chromatography (ethyl acetate:hexane=1:3) to obtain the present compound 3A-1 represented by the following formula 0.33 g.

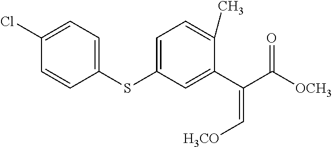 [SMILES: CO/C=C(/C(=O)OC)c1cc(Sc2ccc(Cl)cc2)ccc1C]

Present compound 3A-1: ¹H-NMR (CDCl₃) δ: 7.55 (1H, s), 7.25-7.16 (7H, m), 3.83 (3H, s), 3.70 (3H, s), 2.18 (3H, s).

### Preparation Example 4A

To a mixture of the intermediate compound 25 0.30 g, triphenylphosphine 0.42 g, cyclohexanol 0.19 g, and chloroform 10 mL was added bis(2-methoxyethyl) azodicarboxylate 0.41 g at 0° C., and the mixture was stirred at room temperature for 16 hours. To the resulting mixture was added aqueous saturated sodium hydrogencarbonate solution, and the mixture was extracted with ethyl acetate. The resulting organic layers were washed with water and saturated brine successively, dried over anhydrous magnesium sulfate, and concentrated under reduced pressure. The resulting residue was subjected to a silica gel column chromatography (ethyl acetate:hexane=1:4) to obtain the present compound A-1 represented by the following formula 0.13 g.

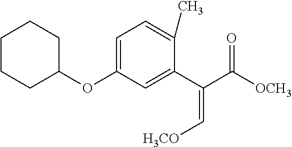 [SMILES: CO/C=C(/C(=O)OC)c1cc(OC2CCCCC2)ccc1C]

Present compound 4A-1: ¹H-NMR (CDCl₃) δ: 7.54 (1H, s), 7.10 (1H, d), 6.77 (1H, dd), 6.66 (1H, d), 4.20-4.14 (1H, m), 3.82 (3H, s), 3.70 (3H, s), 2.10 (3H, s), 2.03-1.94 (2H, m), 1.83-1.73 (2H, m), 1.55-1.29 (6H, m).

Examples of the Present Compound prepared according to the above Process and Preparation Examples are shown below.

Each of Q8A to Q8P represents a substituent wherein a combination of L³, Z¹, Z² and Z³ in the group represented by Q8 represents a combination shown below.
Q8A represents the substituent according to Q8 wherein L³ represents an oxygen atom, Z¹ represents an oxygen atom, Z² represents a fluoromethyl group, and Z³ represents CH;
Q8B represents the substituent according to Q8 wherein L³ represents an oxygen atom, Z¹ represents an oxygen atom, Z² represents a fluoromethyl group, and Z³ represents a nitrogen atom;
Q8C represents the substituent according to Q8 wherein L³ represents a nitrogen atom, Z¹ represents an oxygen atom, Z² represents a fluoromethyl group, and Z³ represents a nitrogen atom;
Q8D represents the substituent according to Q8 wherein L³ represents an oxygen atom, Z¹ represents CH₂, Z² represents a fluoromethyl group, and Z³ represents CH;
Q8E represents the substituent according to Q8 wherein L³ represents an oxygen atom, Z¹ represents CH₂, Z² represents a fluoromethyl group, and Z³ represents a nitrogen atom;
Q8F represents the substituent according to Q8 wherein L³ represents a nitrogen atom, Z¹ represents CH₂, Z² represents a fluoromethyl group, and Z³ represents a nitrogen atom;
Q8G represents the substituent according to Q8 wherein L³ represents an oxygen atom, Z¹ represents NCH₃, Z² represents a fluoromethyl group, and Z³ represents CH;
Q8H represents the substituent according to Q8 wherein L³ represents an oxygen atom, Z¹ represents NCH₃, Z² represents a fluoromethyl group, and Z³ represents a nitrogen atom;
Q8I represents the substituent according to Q8 wherein L³ represents a nitrogen atom, Z¹ represents NCH₃, Z² represents a fluoromethyl group, and Z³ represents a nitrogen atom;
Q8J represents the substituent according to Q8 wherein L³ represents an oxygen atom, Z¹ represents a single bond, Z² represents a fluoromethyl group, and Z³ represents CH;
Q8K represents the substituent according to Q8 wherein L³ represents an oxygen atom, Z¹ represents a single bond, Z² represents a fluoromethyl group, and Z³ represents a nitrogen atom;
Q8L represents the substituent according to Q8 wherein L³ represents a nitrogen atom, Z¹ represents a single bond, Z² represents a fluoromethyl group, and Z³ represents a nitrogen atom;
Q8M represents the substituent according to Q8 wherein L³ represents an oxygen atom, Z¹ represents an oxygen atom, Z² represents a difluoromethyl group, and Z³ represents CH;
Q8N represents the substituent according to Q8 wherein L³ represents an oxygen atom, Z¹ represents CH₂, Z² represents a difluoromethyl group, and Z³ represents CH;
Q8O represents the substituent according to Q8 wherein L³ represents an oxygen atom, Z¹ represents NCH₃, Z² represents a difluoromethyl group, and Z³ represents CH; and
Q8P represents the substituent according to Q8 wherein L³ represents a nitrogen atom, Z¹ represents a single bond, Z² represents a difluoromethyl group, and Z³ represents a nitrogen atom.

A compound represented by formula (1A) (hereinafter, referred to as Compound (1A)):

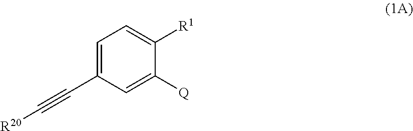

wherein R¹ represents a methyl group, Q represents Q3, R³ represents a methoxy group, and R²⁰ represents any substituent selected from Group X (hereinafter, referred to as Compound group SX1).
Group X: a group consisting of Me, Et, Pr, i-Pr, (CH₂)₃CH₃, CH₂CH(CH₃)₂, CH(CH₃) CH₂CH₃, t-Bu, CH₂c-Pr, (CH₂)₄CH₃, (CH₂)₂CH(CH₃)₂, CH(CH₃)(CH₂)₂CH₃, CH(CH₂CH₃) CH₂CH₃, CH₂CH(CH₃) CH₂CH₃, C(CH₃)₂CH₂CH₃, CH₂t-Bu, CH₂c-Bu, CH₂c-Pen, (CH₂)₂c-Pr, (1-methylcyclopropyl)methyl, (2-methylcyclopropyl) methyl, 1-cyclopropylethyl, (CH₂)₃c-Pr, (CH₂)₅CH₃, (CH₂)₃CH(CH₃)₂, (CH₂)₂CH(CH₃) CH₂CH₃, CH₂CH(CH₃)(CH₂)₂CH₃, CH(CH₃)(CH₂)₃CH₃, CH₂CH(CH₂CH₃)₂, heptyl, CH₂CH═CH₂, CH₂CH═CHCH₃, CH₂CH═C(CH₃)₂, CH₂CH═CF₂, CH₂CH═CCl₂, CH₂CH═CHCH₂CH₃, CH₂CH═CH(CH₂)₂CH₃, CH₂C(CH₃)═CH₂, CH₂C(CH₃)═CHCH₃, CH₂C(CH₃)═C(CH₃)₂, CH₂C(CH₃)═CHCH₂CH₃, CH₂CF═CH₂, CH₂CF═CHCH₃, CH₂CF═C(CH₃)₂, CH₂CF═CF₂, CH₂CF═CHCH₂CH₃, CH₂CF═CH(CH₂)₂CH₃, CH₂CCl═CH₂, CH₂CCl═CHCH₃, CH₂CCl═C(CH₃)₂, CH₂CCl═CCl₂, CH₂CCl═CHCH₂CH₃, CH₂CCl═CH(CH₂)₂CH₃, (CH₂)₂CH═CH₂, (CH₂)₂CH═CHCH₃, (CH₂)₂CH═CHCH₂CH₃, (CH₂)₂CH═C(CH₃)₂, (CH₂)₂C(CH₃)═CH₂, (CH₂)₂C(CH₃)═CHCH₃, (CH₂)₃CH═CH₂, (CH₂)₃C(CH₃)═CH₂, (CH₂)₄CH═CH₂, CH₂C═CH, CH₂C═CCH₃, CH₂C═CCH₂CH₃, CH₂C≡Cc-Pr, (CH₂)₂C═CH, (CH₂)₂C═CCH₃, (CH₂)₂C═CCH₂CH₃, (CH₂)₃C≡CH, (CH₂)₃C═CCH₃, CH₂Cl, CH₂Br, CH₂OCH₃, CH₂OCH₂CH₃, CH₂O(CH₂)₂CH₃, CHF₂, CF₃, CH₂CF₃, CH₂CHF₂, CH₂CH₂CHF₂, CH₂CH₂CF₃, CH₂CF₂CF₃, (CH₂)₂CF₂CF₃, CH₂(CF₂)₂CF₃, (CH₂)₂CF(CF₃)₂, (CH₂) 2 (CF₂)₅CF₃, CF₃, CF₂CHF(CF₃), CF₂CHF(OCF₃), and CH₂CF₂CF₂H.

A compound according to Compound (1A) wherein R¹ represents a chlorine atom, Q represents Q3, R³ represents a methoxy group, and R²⁰ represents any substituent selected from Group X (hereinafter, referred to as Compound group SX2).

A compound according to Compound (1A) wherein R¹ represents a methyl group, Q represents Q4, and R²⁰ represents any substituent selected from Group X (hereinafter, referred to as Compound group SX3).

A compound according to Compound (1A) wherein R¹ represents a chlorine atom, Q represents Q4, and R²⁰ represents any substituent selected from Group X (hereinafter, referred to as Compound group SX4).

A compound according to Compound (1A) wherein R¹ represents a methyl group, Q represents Q5, and R²⁰ represents any substituent selected from Group X (hereinafter, referred to as Compound group SX5).

A compound according to Compound (1A) wherein R¹ represents a chlorine atom, Q represents Q5, and R²⁰ represents any substituent selected from Group X (hereinafter, referred to as Compound group SX6).

A compound according to Compound (1A) wherein R¹ represents a methyl group, Q represents Q6, and R²⁰ represents any substituent selected from Group X (hereinafter, referred to as Compound group SX7).

A compound according to Compound (1A) wherein R¹ represents a chlorine atom, Q represents Q6, and R²⁰ represents any substituent selected from Group X (hereinafter, referred to as Compound group SX8).

A compound according to Compound (1A) wherein R¹ represents a methyl group, Q represents Q7, L² represents an oxygen atom, and R²⁰ represents any substituent selected from Group X (hereinafter, referred to as Compound group SX9).

A compound according to Compound (1A) wherein R¹ represents a chlorine atom, Q represents Q7, L² represents an oxygen atom, and R²⁰ represents any substituent selected from Group X (hereinafter, referred to as Compound group SX10).

A compound according to Compound (1A) wherein R¹ represents a methyl group, Q represents Q7, L² represents NH, and R²⁰ represents any substituent selected from Group X (hereinafter, referred to as Compound group SX11).

A compound according to Compound (1A) wherein R¹ represents a chlorine atom, Q represents Q7, L² represents NH, and R²⁰ represents any substituent selected from Group X (hereinafter, referred to as Compound group SX12).

A compound according to Compound (1A) wherein R¹ represents a methyl group, Q represents Q8A, and R²⁰ represents any substituent selected from Group X (hereinafter, referred to as Compound group SX13).

A compound according to Compound (1A) wherein R¹ represents a chlorine atom, Q represents Q8A, and R²⁰ represents any substituent selected from Group X (hereinafter, referred to as Compound group SX14).

A compound according to Compound (1A) wherein R¹ represents a methyl group, Q represents Q8B, and R²⁰ represents any substituent selected from Group X (hereinafter, referred to as Compound group SX15).

A compound according to Compound (1A) wherein R¹ represents a chlorine atom, Q represents Q8B, and R²⁰ represents any substituent selected from Group X (hereinafter, referred to as Compound group SX16).

A compound according to Compound (1A) wherein R¹ represents a methyl group, Q represents Q8C, and R²⁰ represents any substituent selected from Group X (hereinafter, referred to as Compound group SX17).

A compound according to Compound (1A) wherein R¹ represents a chlorine atom, Q represents Q8C, and R²⁰ represents any substituent selected from Group X (hereinafter, referred to as Compound group SX18).

A compound according to Compound (1A) wherein R¹ represents a methyl group, Q represents Q8D, and R²⁰ represents any substituent selected from Group X (hereinafter, referred to as Compound group SX19).

A compound according to Compound (1A) wherein R¹ represents a methyl group, Q represents Q8E, and R²⁰ represents any substituent selected from Group X (hereinafter, referred to as Compound group SX20).

A compound according to Compound (1A) wherein R¹ represents a methyl group, Q represents Q8F, and R²⁰ represents any substituent selected from Group X (hereinafter, referred to as Compound group SX21).

A compound according to Compound (1A) wherein R¹ represents a hydrogen atom, Q represents Q8G, and R²⁰ represents any substituent selected from Group X (hereinafter, referred to as Compound group SX22).

A compound according to Compound (1A) wherein R¹ represents a hydrogen atom, Q represents Q8H, and R²⁰ represents any substituent selected from Group X (hereinafter, referred to as Compound group SX23).

A compound according to Compound (1A) wherein R¹ represents a hydrogen atom, Q represents Q8I, and R²⁰ represents any substituent selected from Group X (hereinafter, referred to as Compound group SX24).

A compound according to Compound (1A) wherein R¹ represents a methyl group, Q represents Q8J, and R²⁰ represents any substituent selected from Group X (hereinafter, referred to as Compound group SX25).

A compound according to Compound (1A) wherein R¹ represents a chlorine atom, Q represents Q8J, and R²⁰ represents any substituent selected from Group X (hereinafter, referred to as Compound group SX26).

A compound according to Compound (1A) wherein R¹ represents a methyl group, Q represents Q8K, and R²⁰ represents any substituent selected from Group X (hereinafter, referred to as Compound group SX27).

A compound according to Compound (1A) wherein R¹ represents a chlorine atom, Q represents Q8K, and R²⁰ represents any substituent selected from Group X (hereinafter, referred to as Compound group SX28).

A compound according to Compound (1A) wherein R¹ represents a methyl group, Q represents Q8L, and R²⁰ represents any substituent selected from Group X (hereinafter, referred to as Compound group SX29).

A compound according to Compound (1A) wherein R¹ represents a chlorine atom, Q represents Q8L, and R²⁰ represents any substituent selected from Group X (hereinafter, referred to as Compound group SX30).

A compound according to Compound (1A) wherein R¹ represents a methyl group, Q represents Q8M, and R²⁰ represents any substituent selected from Group X (hereinafter, referred to as Compound group SX31).

A compound according to Compound (1A) wherein R¹ represents a methyl group, Q represents Q8N, and R²⁰ represents any substituent selected from Group X (hereinafter, referred to as Compound group SX32).

A compound according to Compound (1A) wherein R¹ represents a methyl group, Q represents Q8O, and R²⁰ represents any substituent selected from Group X (hereinafter, referred to as Compound group SX33).

A compound according to Compound (1A) wherein R¹ represents a methyl group, Q represents Q8P, and R²⁰ represents any substituent selected from Group X (hereinafter, referred to as Compound group SX34).

A compound represented by formula (1B) (hereinafter, referred to as Compound (1B)):

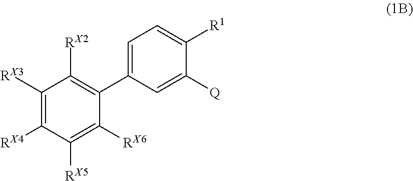

wherein R¹ represents a methyl group, Q represents Q3, R³ represents a methoxy group, and a combination of R^{X2}, R^{X3}, R^{X4}, R^{X5} and R^{X6} represents any combination described in Combination A (hereinafter, referred to as Compound group SX35).

Combination A consists of substituent numbers ZA1 to ZA312. Substituent numbers ZA1 to ZA312 represent combinations of R^{X2}, R^{X3}, R^{X4}, R^{X5} and R^{X6} according to Compound (1B), compounds represented by formula (1D), compounds represented by formula (1E), compounds represented by formula (1F), compounds represented by formula (1T), compounds represented by formula (1K), compounds represented by formula (1M), compounds represented by formula (1N), compounds represented by formula (1P), compounds represented by formula (1W), compounds represented by formula (1Y), compounds represented by formula (1Z), compounds represented by formula (2F), compounds represented by formula (2H), compounds represented by formula (2J), compounds represented by formula (2L), compounds represented by formula (2N), compounds represented by formula (2P), compounds represented by formula (3A), compounds represented by formula (3D), compounds represented by formula (3F), compounds represented by formula (3G), and compounds represented by formula (3H). And hereinafter, they are referred to as [substituent number; R^{X2}, R^{X3}, R^{X4}, R^{X5}, R^{X6}]. For example, the substituent number ZA2 refers to a combination wherein R^{X2} represents a methyl group, and each of R^{X3}, R^{X4}, R^{X5} and R^{X6} represents a hydrogen atom.
Combination A: [ZA1;H,H,H,H,H], [ZA2;Me,H,H,H,H], [ZA3;F,H,H,H,H], [ZA4;Cl,H, H,H,H], [ZA5;OMe,H,H,H,H], [ZA6;CF₃,H,H,H,H], [ZA7;H,Me,H,H,H], [ZA8;H,Et,H,H,H], [ZA9;H,Pr,H,H,H], [ZA10;H,i-Pr,H,H,H], [ZA11;H,t-Bu,H,H,H], [ZA12;H,OMe,H,H,H], [ZA13;H,OEt,H,H,H], [ZA14;H,OPr,H,H,H], [ZA15;H,Oi-Pr,H,H,H], [ZA16;H,CF₃,H,H,H], [ZA17;H,CF₂H,H,H,H], [ZA18;H,CF H₂,H,H,H], [ZA19;H,F,H,H,H], [ZA20;H,Cl,H,H,H], [ZA21;H,Br,H,H,H], [ZA22;H,CN,H,H,H], [ZA23;H,Ph,H,H,H], [ZA24;H,OPh,H,H,H], [ZA25;H,c-Pr,H,H,H], [ZA26;H,c-Pen,H,H,H], [ZA27;H,c-Hex,H,H,H], [ZA28;H,H,Me,H,H], [ZA29;H,H,Et,H,H], [ZA30;H,H,Pr,H,H], [ZA31;H,H,i-Pr,H,H1, [ZA32;H,H,t-Bu,H,H], [ZA33;H,H,OMe,H,H], [ZA34;H,H,OEt,H,H], [ZA35;H,H,OPr,H,H], [ZA36;H,H,Oi-Pr,H,H], [ZA37;H,H,CF₃,H,H], [ZA38;H,H,CF₂H,H,H], [ZA39;H,H,CF H₂,H,H], [ZA40;H,H,F,H,H], [ZA41;H,H,Cl,H,H], [ZA42;H,H,Br,H,H], [ZA43;H,H,CN,H,H], [ZA44;H,H,Ph,H,H], [ZA45;H,H,OPh,H,H], [Z A46;H,H,c-Pr,H,H], [ZA47;H,H,c-Pen,H,H], [ZA48;H,H,c-Hex,H,H], [ZA49;H,H,H,H,F], [ZA50;Me,H,H,H,F], [ZA51;F,H,H,H,F], [ZA52;Cl,H,H,H,F], [ZA53;H,Me,H,H,F], [ZA54;H,Et,H,H,F], [ZA 55;H,Pr,H,H,F], [ZA56;H,i-Pr,H,H,F], [ZA57;H,t-Bu,H,H,F], [ZA58;H,OMe,H,H,F], [ZA59;H,OEt,H,H,F], [ZA60;H,OPr,H,H,F], [ZA61;H,Oi-Pr,H,H,F], [ZA62;H,CF₃,H,H,F], [ZA63;H,CF₂H,H,H,F], [ZA64;H,CF H₂,H,H,F], [ZA65;H,F,H,H,F], [ZA66;H,Cl,H,H,F], [ZA67;H,Br,H,H,F], [ZA68;H,CN,H,H,F], [ZA69;H,Ph,H,H,F], [ZA70;H,OPh,H,H,F], [ZA71;H,c-Pr,H,H,F], [ZA72;H,c-Pen,H,H,F], [ZA73;H,c-Hex,H,H,F], [ZA74;H,H,Me,H,F], [ZA75;H,H,Et,H,F], [ZA76;H,H,Pr,H,F], [ZA77;H,H,i-Pr,H,F], [ZA78;H,H,t-Bu,H,F], [ZA79;H,H,OMe,H,F], [ZA80;H,H,OEt,H,F], [ZA81;H,H,OPr,H,F], [ZA82;H,H,Oi-Pr,H,F], [ZA83;H,H,CF₃,H,F], [ZA84;H,H,CF₂H,H,F], [ZA85;H,H,CF H₂,H,F], [ZA86;H,H,F,H,F], [ZA87;H,H,Cl,H,F], [ZA88;H,H,Br,H,F], [ZA89;H,H,CN,H,F], [ZA90;H,H,Ph,H,F], [ZA91;H,H,OPh,H,F], [Z A92;H,H,c-Pr,H,F], [ZA93;H,H,c-Pen,H,F], [ZA94;H,H,c-Hex,H,F], [ZA95;H,H,H,H,Cl], [ZA96;Me,H,H,H,Cl], [ZA97;F,H,H,H,Cl], [ZA98;Cl,H,H,H,Cl], [ZA99;H,Me,H,H,Cl], [ZA100;H,Et,H,H, Cl], [ZA101;H,Pr,H,H,Cl], [ZA102;H,i-Pr,H,H,Cl], [ZA103;H,t-Bu,H,H,Cl], [ZA104;H,OMe,H,H,Cl], [ZA105;H,OEt,H,H,Cl], [ZA106;H,OPr,H,H,Cl], [ZA107;H,Oi-Pr,H,H,Cl], [ZA108;H,CF₃,H,H,Cl], [ZA109;H,CF₂H,H,H,Cl], [ZA110;H,CFH₂,H,H,Cl], [ZA111;H,F,H,H,Cl], [ZA112;H,Cl,H,H,Cl], [ZA 113;H,Br,H,H,Cl], [ZA114;H,CN,H,H,Cl], [ZA115;H,Ph,H,H,Cl], [Z A116;H,OPh,H,H,Cl], [ZA117;H,c-Pr,H,H,Cl], [ZA118;H,H,H,F,F], [ZA119;F,H,H,F,F], [ZA120;H,H,M e,H,Cl], [ZA121;H,H,Et,H,Cl], [ZA122;H,H,Pr,H,Cl], [ZA123;H,H, i-Pr,H,Cl], [ZA124;H,H,t-Bu,H,Cl], [ZA125;H,H,OMe,H,Cl], [ZA126;H,H,OEt,H,Cl], [ZA127;H,H,OPr,H,Cl], [ZA128;H,H,Oi-Pr,H,Cl], [ZA129;H,H,CF₃,H,Cl], [ZA130;H,H,CF₂H,H,Cl], [ZA131; H,H,CFH₂,H,Cl], [ZA132;H,H,F,H,Cl], [ZA133;H,H,Cl,H,Cl], [ZA13 4;H,H,Br,H,Cl], [ZA135;H,H,CN,H,Cl], [ZA136;H,H,Ph,H,Cl], [ZA137;H,H,OPh,H,Cl], [ZA138;H,H,c-Pr,H,Cl], [ZA139;H,H,c-Pen,H,Cl], [ZA140;H,H,c-Hex,H,Cl], [ZA141;H,H,H,H,Me], [ZA142;Me,H,H,H,Me], [ZA143;F,H,H,H,Me], [ZA144;Cl,H,H,H,Me], [ZA145;H,Me,H,H,Me], [ZA146;H,E t,H,H,Me], [ZA147;H,Pr,H,H,Me], [ZA148;H,i-Pr,H,H,Me], [ZA149;H,t-Bu,H,H,Me], [ZA150;H,OMe,H,H,Me], [ZA151;H,OEt,H,H,Me], [ZA152;H,OPr,H,H,Me], [ZA153;H,Oi-Pr,H,H,Me], [ZA154;H,CF₃,H,H,Me], [ZA155;H,CF₂H,H,H,Me], [ZA15 6;H,CFH₂,H,H,Me], [ZA157;H,F,H,H,Me], [ZA158;H,Cl,H,H,Me], [ZA 159;H,Br,H,H,Me], [ZA160;H,CN,H,H,Me], [ZA161;H,Ph,H,H,Me], [Z A162;H,OPh,H,H,Me], [ZA163;H,c-Pr,H,H,Me], [ZA164;H,c-Pen,H,H,Me], [ZA165;H,c-Hex,H,H,Me], [ZA166;H,H,Me,H,Me], [ZA167;H,H,Et,H,Me], [ZA168; H,H,Pr,H,Me], [ZA169;H,H,i-Pr,H,Me], [ZA170;H,H,t-Bu,H,Me], [ZA171;H,H,OMe,H,Me], [ZA172;H,H,OEt,H,Me], [ZA173;H,H,OPr,H,Me], [ZA174;H,H,Oi-Pr,H,Me], [ZA175;H,H,CF₃,H,Me], [ZA176;H,H,CF₂H,H,Me], [ZA177; H,H,CFH₂,H,Me], [ZA178;H,H,F,H,Me], [ZA179;H,H,Cl,H,Me], [ZA18 0;H,H,Br,H,Me], [ZA181;H,H,CN,H,Me], [ZA182;H,H,Ph,H,Me], [ZA1 83;H,H,OPh,H,Me], [ZA184;H,H,c-Pr,H,Me], [ZA185;H,H,c-Pen,H,Me], [ZA186;H,H,c-Hex,H,Me], [ZA187;H,H,H,H,OMe], [ZA188;Me,H,H,H,OMe], [ZA189;F,H,H,H,OMe], [ZA190;Cl,H,H,H,OMe], [ZA191;H,Me,H,H,OMe], [ZA19 2;H,Et,H,H,OMe], [ZA193;H,Pr,H,H,OMe], [ZA194;H,i-Pr,H,H,OMe], [ZA195;H,t-Bu,H,H,OMe], [ZA196;H,OMe,H,H,OMe], [ZA197;H,OEt,H,H,OMe], [ZA 198;H,OPr,H,H,OMe], [ZA199;H,Oi-Pr,H,H,OMe], [ZA200;H,CF₃,H,H,OMe], [ZA201;H,CF₂H,H,H,OMe], [ZA202;H,CFH₂,H,H,OMe], [ZA203;H,F,H, H,OMe], [ZA204;H,Cl,H,H,OMe], [ZA205;H,Br,H,H,OMe], [ZA206;H,C N,H,H,OMe], [ZA207;H,Ph,H,H,OMe], [ZA208;H,OPh,H,H,OMe], [ZA20 9;H,c-Pr,H,H,OMe], [ZA210;H,c-Pen,H,H,OMe], [ZA211;H,c-Hex,H,H,OMe], [ZA212;H,H,Me,H,OMe], [ZA213;H,H,Et,H,OMe], [ZA2 14;H,H,Pr,H,OMe], [ZA215;H,H,i-Pr,H,OMe], [ZA216;H,H,t-Bu,H,OMe], [ZA217;H,H,OMe,H,OMe], [ZA218;H,H,OEt,H,OMe], [ZA21 9;H,H,OPr,H,OMe], [ZA220;H,H,Oi-Pr,H,OMe], [ZA221;H,H,CF₃,H,OMe], [ZA222;H,H,CF₂H,H,OMe], [ZA2 23;H,H,CFH₂,H,OMe], [ZA224;H,H,F,H,OMe], [ZA225;H,H,Cl,H,OMe], [ZA226;H,H,Br,H,OMe], [ZA227;H,H,CN,H,OMe], [ZA228;H,H,Ph,H, OMe], [ZA229;H,H,OPh,H,OMe], [ZA230;H,H,c-Pr,H,OMe], [ZA231;H,H,c-Pen,H,OMe], [ZA232;H,H,c-Hex,H,OMe], [ZA233;H,H,H,H,CF₃], [ZA234;Me,H,H,H,CF₃], [ZA235; F,H,H,H,CF₃], [ZA236;Cl,H,H,H,CF₃], [ZA237;H,Me,H,H,CF₃], [ZA23 8;H,Et,H,H,CF₃], [ZA239;H,Pr,H,H,CF₃], [ZA240;H,i-Pr,H,H,CF₃], [ZA241;H,t-Bu,H,H,CF₃], [ZA242;H,OMe,H,H,CF₃], [ZA243;H,OEt,H,H,CF₃], [ZA2 44;H,OPr,H,H,CF₃], [ZA245;H,Oi-Pr,H,H,CF₃], [ZA246;H,CF₃,H,H,CF₃], [ZA247;H,CF₂H,H,H,CF₃], [ZA 248;H,CFH₂,H,H,CF₃], [ZA249;H,F,H,H,CF₃], [ZA250;H,Cl,H,H,CF₃], [ZA251;H,Br,H,H,CF₃], [ZA252;H,CN,H,H,CF₃], [ZA253;H,Ph,H,H, CF₃], [ZA254;H,OPh,H,H,CF₃], [ZA255;H,c-Pr,H,H,CF₃], [ZA256;H,c-Pen,H,H,CF₃], [ZA257;H,c-Hex,H,H,CF₃], [ZA258;H,H,Me,H,CF₃], [ZA259;H,H,Et,H,CF₃], [ZA26 0;H,H,Pr,H,CF₃], [ZA261;H,H,i-Pr,H,CF₃], [ZA262;H,H,t-Bu,H,CF₃], [ZA263;H,H,OMe,H,CF₃], [ZA264;H,H,OEt,H,CF₃], [ZA265;H,H,OPr,H,CF₃], [ZA266;H,H,Oi-Pr,H,CF₃], [ZA267;H,H,CF₃,H,CF₃], [ZA268;H,H,CF₂H,H,CF₃], [ZA26 9;H,H,CFH₂,H,CF₃], [ZA270;H,H,F,H,CF₃], [ZA271;H,H,Cl,H,CF₃], [ZA272;H,H,Br,H,CF₃], [ZA273;H,H,CN,H,CF₃], [ZA274;H,H,Ph,H,CF₃], [ZA275;H,H,OPh,H,CF₃], [ZA276;H,H,c-Pr,H,CF₃], [ZA277;H,H,c-Pen,H,CF₃], [ZA278;H,H,c-Hex,H,CF₃], [ZA279;H,F,F,H,H], [ZA280;H,F,H,F,H], [ZA281;H,F,F,F,H], [ZA282;F,F,F,H,H], [ZA283;F,F,H,F,H], [ZA284;F,H,F,H,F], [ZA285;F,F,F,F,F], [ZA286;F,F,H,F,F], [ZA287;H,OMe,H,OMe,H], [ZA288;H,F,Cl,H,H], [ZA289;H,F,Me,H,H], [ZA290;H,F,OMe,H,H], [ZA291;H,F,CF₃,H,H], [ZA292;H,Cl,F,H,H], [ZA293;H,Cl,Cl,H,H], [ZA294;H,Cl,Me,H,H], [ZA295;H,Cl,OMe,H,H], [ZA296;H,Cl,CF₃,H,H], [ZA297;H,Me,F,H,H], [ZA298;H,Me,Cl,H,H], [ZA299;H,Me,Me,H,H], [ZA300;H,Me,OMe,H,H], [ZA301;H,Me,CF₃,H,H], [ZA302;H,OMe,F,H,H], [ZA303;H,OMe,Cl,H,H], [ZA304;H,OMe,Me,H,H], [ZA305;H,OMe,OMe,H,H], [ZA306;H,OMe,CF₃,H,H], [ZA307;H,CF₃,F,H,H], [ZA308; H,CF₃,Cl,H,H], [ZA309;H,CF₃,F,H,H], [ZA310;H,CF₃,Cl,H,H], [ZA31 1;H,CF₃,F,H,H], [ZA312;H,Cl,H,Cl,H]

A compound according to Compound (1B) wherein R¹ represents a chlorine atom, Q represents Q3, R³ represents a methoxy group, and a combination of R^{X2}, R^{X3}, R^{X4}, R^{X5} and R^{X6} represents any combination described in Combination A (hereinafter, referred to as Compound group SX36).

A compound according to Compound (1B) wherein R¹ represents a methyl group, Q represents Q4, and a combination of R^{X2}, R^{X3}, R^{X4}, R^{X5} and R^{X6} represents any combination described in Combination A (hereinafter, referred to as Compound group SX37).

A compound according to Compound (1B) wherein R¹ represents a chlorine atom, Q represents Q4, and a combination of R^{X2}, R^{X3}, R^{X4}, R^{X5} and R^{X6} represents any combination described in Combination A (hereinafter, referred to as Compound group SX38).

A compound according to Compound (1B) wherein R¹ represents a methyl group, Q represents Q5, and a combination of R^{X2}, R^{X3}, R^{X4}, R^{X5} and R^{X6} represents any combination described in Combination A (hereinafter, referred to as Compound group SX39).

A compound according to Compound (1B) wherein R¹ represents a chlorine atom, Q represents Q5, and a combination of R^{X2}, R^{X3}, R^{X4}, R^{X5} and R^{X6} represents any combination described in Combination A (hereinafter, referred to as Compound group SX40).

A compound according to Compound (1B) wherein R¹ represents a methyl group, Q represents Q6, and a combination of R^{X2}, R^{X3}, R^{X4}, R^{X5} and R^{X6} represents any combination described in Combination A (hereinafter, referred to as Compound group SX41).

A compound according to Compound (1B) wherein R¹ represents a chlorine atom, Q represents Q6, and a combination of R^{X2}, R^{X3}, R^{X4}, R^{X5} and R^{X6} represents any combination described in Combination A (hereinafter, referred to as Compound group SX42).

A compound according to Compound (1B) wherein R¹ represents a methyl group, Q represents Q7, L² represents an oxygen atom, and a combination of R^{X2}, R^{X3}, Rx, R^{X5} and R^{X6} represents any combination described in Combination A (hereinafter, referred to as Compound group SX43).

A compound according to Compound (1B) wherein R¹ represents a chlorine atom, Q represents Q7, L² represents an oxygen atom, and a combination of R^{X2}, R^{X3}, Rx, R^{X5} and R^{X6} represents any combination described in Combination A (hereinafter, referred to as Compound group SX44).

A compound according to Compound (1B) wherein R¹ represents a methyl group, Q represents Q7, L² represents NH, and a combination of R^{X2}, R^{X3}, Rx, R^{X5} and R^{X6} represents any combination described in Combination A (hereinafter, referred to as Compound group SX45).

A compound according to Compound (1B) wherein R¹ represents a chlorine atom, Q represents Q7, L² represents NH, and a combination of R^{X2}, R^{X3}, Rx, R^{X5} and R^{X6} represents any combination described in Combination A (hereinafter, referred to as Compound group SX46).

A compound according to Compound (1B) wherein R¹ represents a methyl group, Q represents Q8A, and a combination of R^{X2}, R^{X3}, Rx, R^{X5} and R^{X6} represents any combination described in Combination A (hereinafter, referred to as Compound group SX47).

A compound according to Compound (1B) wherein R¹ represents a chlorine atom, Q represents Q8A, and a combination of R^{X2}, R^{X3}, R^{X4}, R^{X5} and R^{X6} represents any combination described in Combination A (hereinafter, referred to as Compound group SX48).

A compound according to Compound (1B) wherein R¹ represents a methyl group, Q represents Q8B, and a combination of R^{X2}, R^{X3}, R^{X4}, R^{X5} and R^{X6} represents any combination described in Combination A (hereinafter, referred to as Compound group SX49).

A compound according to Compound (1B) wherein R¹ represents a chlorine atom, Q represents Q8B, and a combination of R^{X2}, R^{X3}, R^{X4}, R^{X5} and R^{X6} represents any combination described in Combination A (hereinafter, referred to as Compound group SX50).

A compound according to Compound (1B) wherein R¹ represents a methyl group, Q represents Q8C, and a combination of R^{X2}, R^{X3}, R^{X4}, R^{X5} and R^{X6} represents any combination described in Combination A (hereinafter, referred to as Compound group SX51).

A compound according to Compound (1B) wherein R¹ represents a chlorine atom, Q represents Q8C, and a combination of R^{X2}, R^{X3}, R^{X4}, R^{X5} and R^{X6} represents any combination described in Combination A (hereinafter, referred to as Compound group SX52).

A compound according to Compound (1B) wherein R¹ represents a methyl group, Q represents Q8D, and a combination of R^{X2}, R^{X3}, R^{X4}, R^{X5} and R^{X6} represents any combination described in Combination A (hereinafter, referred to as Compound group SX53).

A compound according to Compound (1B) wherein R¹ represents a methyl group, Q represents Q8E, and a combination of R^{X2}, R^{X3}, R^{X4}, R^{X5} and R^{X6} represents any combination described in Combination A (hereinafter, referred to as Compound group SX54).

A compound according to Compound (1B) wherein R¹ represents a methyl group, Q represents Q8F, and a combination of R^{X2}, R^{X3}, R^{X4}, R^{X5} and R^{X6} represents any combination described in Combination A (hereinafter, referred to as Compound group SX55).

A compound according to Compound (1B) wherein R¹ represents a hydrogen atom, Q represents Q8G, and a combination of R^{X2}, R^{X3}, R^{X4}, R^{X5} and R^{X6} represents any combination described in Combination A (hereinafter, referred to as Compound group SX56).

A compound according to Compound (1B) wherein R¹ represents a hydrogen atom, Q represents Q8H, and a combination of R^{X2}, R^{X3}, R^{X4}, R^{X5} and R^{X6} represents any combination described in Combination A (hereinafter, referred to as Compound group SX57).

A compound according to Compound (1B) wherein R¹ represents a hydrogen atom, Q represents Q8I, and a combination of R^{X2}, R^{X3}, R^{X4}, R^{X5} and R^{X6} represents any combination described in Combination A (hereinafter, referred to as Compound group SX58).

A compound according to Compound (1B) wherein R¹ represents a methyl group, Q represents Q8J, and a combination of R^{X2}, R^{X3}, R^{X4}, R^{X5} and R^{X6} represents any combination described in Combination A (hereinafter, referred to as Compound group SX59).

A compound according to Compound (1B) wherein R¹ represents a chlorine atom, Q represents Q8J, and a combination of R^{X2}, R^{X3}, R^{X4}, R^{X5} and R^{X6} represents any combination described in Combination A (hereinafter, referred to as Compound group SX60).

A compound according to Compound (1B) wherein R¹ represents a methyl group, Q represents Q8K, and a combination of R^{X2}, R^{X3}, R^{X4}, R^{X5} and R^{X6} represents any combination described in Combination A (hereinafter, referred to as Compound group SX61).

A compound according to Compound (1B) wherein R¹ represents a chlorine atom, Q represents Q8K, and a combination of R^{X2}, R^{X3}, R^{X4}, R^{X5} and R^{X6} represents any combination described in Combination A (hereinafter, referred to as Compound group SX62).

A compound according to Compound (1B) wherein R¹ represents a methyl group, Q represents Q8L, and a combination of R^{X2}, R^{X3}, R^{X4}, R^{X5} and R^{X6} represents any combination described in Combination A (hereinafter, referred to as Compound group SX63).

A compound according to Compound (1B) wherein R¹ represents a chlorine atom, Q represents Q8L, and a combination of R^{X2}, R^{X3}, R^{X4}, R^{X5} and R^{X6} represents any combination described in Combination A (hereinafter, referred to as Compound group SX64).

A compound according to Compound (1B) wherein R¹ represents a methyl group, Q represents Q8M, and a combination of R^{X2}, R^{X3}, R^{X4}, R^{X5} and R^{X6} represents any combination described in Combination A (hereinafter, referred to as Compound group SX65).

A compound according to Compound (1B) wherein R¹ represents a methyl group, Q represents Q8N, and a combination of R^{X2}, R^{X3}, R^{X4}, R^{X5} and R^{X6} represents any combination described in Combination A (hereinafter, referred to as Compound group SX66).

A compound according to Compound (1B) wherein R¹ represents a methyl group, Q represents Q8O, and a combination of R^{X2}, R^{X3}, R^{X4}, R^{X5} and R^{X6} represents any combination described in Combination A (hereinafter, referred to as Compound group SX67).

A compound according to Compound (1B) wherein R¹ represents a methyl group, Q represents Q8P, and a combination of R^{X2}, R^{X3}, R^{X4}, R^{X5} and R^{X6} represents any combination described in Combination A (hereinafter, referred to as Compound group SX68).

A compound represented by formula (1C) (hereinafter, referred to as Compound (1C)):

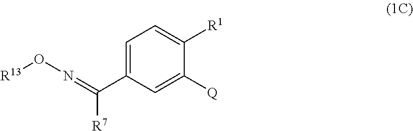

wherein R¹ represents a methyl group, Q represents Q3, R³ represents a methoxy group, R⁷ represents a methyl group, and R¹³ represents any substituent selected from Group X (hereinafter, referred to as Compound group SX69).

A compound according to Compound (1C) wherein R¹ represents a chlorine atom, Q represents Q3, R³ represents a methoxy group, R⁷ represents a methyl group, and R¹³ represents any substituent selected from Group X (hereinafter, referred to as Compound group SX70).

A compound according to Compound (1C) wherein R¹ represents a methyl group, Q represents Q4, R⁷ represents a methyl group, and R¹³ represents any substituent selected from Group X (hereinafter, referred to as Compound group SX71).

A compound according to Compound (1C) wherein R¹ represents a chlorine atom, Q represents Q4, R⁷ represents a methyl group, and R¹³ represents any substituent selected from Group X (hereinafter, referred to as Compound group SX72).

A compound according to Compound (1C) wherein R¹ represents a methyl group, Q represents Q5, R⁷ represents a methyl group, and R¹³ represents any substituent selected from Group X (hereinafter, referred to as Compound group SX73).

A compound according to Compound (1C) wherein R¹ represents a chlorine atom, Q represents Q5, R⁷ represents a methyl group, and R¹³ represents any substituent selected from Group X (hereinafter, referred to as Compound group SX74).

A compound according to Compound (1C) wherein R¹ represents a methyl group, Q represents Q6, R⁷ represents a methyl group, and R¹³ represents any substituent selected from Group X (hereinafter, referred to as Compound group SX75).

A compound according to Compound (1C) wherein R¹ represents a chlorine atom, Q represents Q6, R⁷ represents a methyl group, and R¹³ represents any substituent selected from Group X (hereinafter, referred to as Compound group SX76).

A compound according to Compound (1C) wherein R¹ represents a methyl group, Q represents Q7, L² represents an oxygen atom, R⁷ represents a methyl group, and R¹³ represents any substituent selected from Group X (hereinafter, referred to as Compound group SX77).

A compound according to Compound (1C) wherein R¹ represents a chlorine atom, Q represents Q7, L² represents an oxygen atom, R⁷ represents a methyl group, and R¹³ represents any substituent selected from Group X (hereinafter, referred to as Compound group SX78).

A compound according to Compound (1C) wherein R¹ represents a methyl group, Q represents Q7, L² represents NH, R⁷ represents a methyl group, and R¹³ represents any substituent selected from Group X (hereinafter, referred to as Compound group SX79).

A compound according to Compound (1C) wherein R¹ represents a chlorine atom, Q represents Q7, L² represents NH, R⁷ represents a methyl group, and R¹³ represents any substituent selected from Group X (hereinafter, referred to as Compound group SX80).

A compound according to Compound (1C) wherein R¹ represents a methyl group, Q represents Q8A, R⁷ represents a methyl group, and R¹³ represents any substituent selected from Group X (hereinafter, referred to as Compound group SX81).

A compound according to Compound (1C) wherein R¹ represents a chlorine atom, Q represents Q8A, R⁷ represents a methyl group, and R¹³ represents any substituent selected from Group X (hereinafter, referred to as Compound group SX82).

A compound according to Compound (1C) wherein R¹ represents a methyl group, Q represents Q8B, R⁷ represents a methyl group, and R¹³ represents any substituent selected from Group X (hereinafter, referred to as Compound group SX83).

A compound according to Compound (1C) wherein R¹ represents a chlorine atom, Q represents Q8B, R⁷ represents a methyl group, and R¹³ represents any substituent selected from Group X (hereinafter, referred to as Compound group SX84).

A compound according to Compound (1C) wherein R¹ represents a methyl group, Q represents Q8C, R⁷ represents a methyl group, and R¹³ represents any substituent selected from Group X (hereinafter, referred to as Compound group SX85).

A compound according to Compound (1C) wherein R¹ represents a chlorine atom, Q represents Q8C, R⁷ represents a methyl group, and R¹³ represents any substituent selected from Group X (hereinafter, referred to as Compound group SX86).

A compound according to Compound (1C) wherein R¹ represents a methyl group, Q represents Q8D, R⁷ represents a methyl group, and R¹³ represents any substituent selected from Group X (hereinafter, referred to as Compound group SX87).

A compound according to Compound (1C) wherein R¹ represents a methyl group, Q represents Q8E, R⁷ represents a methyl group, and R¹³ represents any substituent selected from Group X (hereinafter, referred to as Compound group SX88).

A compound according to Compound (1C) wherein R¹ represents a methyl group, Q represents Q8F, R⁷ represents a methyl group, and R¹³ represents any substituent selected from Group X (hereinafter, referred to as Compound group SX89).

A compound according to Compound (1C) wherein R¹ represents a hydrogen atom, Q represents Q8G, R⁷ represents a methyl group, and R¹³ represents any substituent selected from Group X (hereinafter, referred to as Compound group SX90).

A compound according to Compound (1C) wherein R¹ represents a hydrogen atom, Q represents Q8H, R⁷ represents a methyl group, and R¹³ represents any substituent selected from Group X (hereinafter, referred to as Compound group SX91).

A compound according to Compound (1C) wherein R¹ represents a hydrogen atom, Q represents Q8I, R⁷ represents a methyl group, and R¹³ represents any substituent selected from Group X (hereinafter, referred to as Compound group SX92).

A compound according to Compound (1C) wherein R¹ represents a methyl group, Q represents Q8J, R⁷ represents a methyl group, and R¹³ represents any substituent selected from Group X (hereinafter, referred to as Compound group SX93).

A compound according to Compound (1C) wherein R¹ represents a chlorine atom, Q represents Q8J, R⁷ represents a methyl group, and R¹³ represents any substituent selected from Group X (hereinafter, referred to as Compound group SX94).

A compound according to Compound (1C) wherein R¹ represents a methyl group, Q represents Q8K, R⁷ represents a methyl group, and R¹³ represents any substituent selected from Group X (hereinafter, referred to as Compound group SX95).

A compound according to Compound (1C) wherein R¹ represents a chlorine atom, Q represents Q8K, R⁷ represents a methyl group, and R¹³ represents any substituent selected from Group X (hereinafter, referred to as Compound group SX96).

A compound according to Compound (1C) wherein R¹ represents a methyl group, Q represents Q8L, R⁷ represents a methyl group, and R¹³ represents any substituent selected from Group X (hereinafter, referred to as Compound group SX97).

A compound according to Compound (1C) wherein R¹ represents a chlorine atom, Q represents Q8L, R⁷ represents a methyl group, and R¹³ represents any substituent selected from Group X (hereinafter, referred to as Compound group SX98).

A compound according to Compound (1C) wherein R¹ represents a methyl group, Q represents Q8M, R⁷ represents a methyl group, and R¹³ represents any substituent selected from Group X (hereinafter, referred to as Compound group SX99).

A compound according to Compound (1C) wherein R¹ represents a methyl group, Q represents Q8N, R⁷ represents a methyl group, and R¹³ represents any substituent selected from Group X (hereinafter, referred to as Compound group SX100).

A compound according to Compound (1C) wherein R¹ represents a methyl group, Q represents Q8O, R⁷ represents a methyl group, and R¹³ represents any substituent selected from Group X (hereinafter, referred to as Compound group SX101).

A compound according to Compound (1C) wherein R¹ represents a methyl group, Q represents Q8P, R⁷ represents a methyl group, and R¹³ represents any substituent selected from Group X (hereinafter, referred to as Compound group SX102).

A compound according to Compound (1C) wherein R¹ represents a methyl group, Q represents Q3, R³ represents a methoxy group, R⁷ represents an ethyl group, and R¹³ represents any substituent selected from Group X (hereinafter, referred to as Compound group SX103).

A compound according to Compound (1C) wherein R¹ represents a chlorine atom, Q represents Q3, R³ represents a methoxy group, R⁷ represents an ethyl group, and R¹³ represents any substituent selected from Group X (hereinafter, referred to as Compound group SX104).

A compound according to Compound (1C) wherein R¹ represents a methyl group, Q represents Q4, R⁷ represents an ethyl group, and R¹³ represents any substituent selected from Group X (hereinafter, referred to as Compound group SX105).

A compound according to Compound (1C) wherein R¹ represents a chlorine atom, Q represents Q4, R⁷ represents an ethyl group, and R¹³ represents any substituent selected from Group X (hereinafter, referred to as Compound group SX106).

A compound according to Compound (1C) wherein R¹ represents a methyl group, Q represents Q5, R⁷ represents an ethyl group, and R¹³ represents any substituent selected from Group X (hereinafter, referred to as Compound group SX107).

A compound according to Compound (1C) wherein R¹ represents a chlorine atom, Q represents Q5, R⁷ represents an ethyl group, and R¹³ represents any substituent selected from Group X (hereinafter, referred to as Compound group SX108).

A compound according to Compound (1C) wherein R¹ represents a methyl group, Q represents Q6, R⁷ represents an ethyl group, and R¹³ represents any substituent selected from Group X (hereinafter, referred to as Compound group SX109).

A compound according to Compound (1C) wherein R¹ represents a chlorine atom, Q represents Q6, R⁷ represents an ethyl group, and R¹³ represents any substituent selected from Group X (hereinafter, referred to as Compound group SX110).

A compound according to Compound (1C) wherein R¹ represents a methyl group, Q represents Q7, L² represents an oxygen atom, R⁷ represents an ethyl group, and R¹³ represents any substituent selected from Group X (hereinafter, referred to as Compound group SX111).

A compound according to Compound (1C) wherein R¹ represents a chlorine atom, Q represents Q7, L² represents an oxygen atom, R⁷ represents an ethyl group, and R¹³ represents any substituent selected from Group X (hereinafter, referred to as Compound group SX112).

A compound according to Compound (1C) wherein R¹ represents a methyl group, Q represents Q7, L² represents NH, R⁷ represents an ethyl group, and R¹³ represents any substituent selected from Group X (hereinafter, referred to as Compound group SX113).

A compound according to Compound (1C) wherein R¹ represents a chlorine atom, Q represents Q7, L² represents NH, R⁷ represents an ethyl group, and R¹³ represents any substituent selected from Group X (hereinafter, referred to as Compound group SX114).

A compound according to Compound (1C) wherein R¹ represents a methyl group, Q represents Q8A, R⁷ represents an ethyl group, and R¹³ represents any substituent selected from Group X (hereinafter, referred to as Compound group SX115).

A compound according to Compound (1C) wherein R¹ represents a chlorine atom, Q represents Q8A, R⁷ represents an ethyl group, and R¹³ represents any substituent selected from Group X (hereinafter, referred to as Compound group SX116).

A compound according to Compound (1C) wherein R¹ represents a methyl group, Q represents Q8B, R⁷ represents an ethyl group, and R¹³ represents any substituent selected from Group X (hereinafter, referred to as Compound group SX117).

A compound according to Compound (1C) wherein R¹ represents a chlorine atom, Q represents Q8B, R⁷ represents an ethyl group, and R¹³ represents any substituent selected from Group X (hereinafter, referred to as Compound group SX118).

A compound according to Compound (1C) wherein R¹ represents a methyl group, Q represents Q8C, R⁷ represents an ethyl group, and R¹³ represents any substituent selected from Group X (hereinafter, referred to as Compound group SX119).

A compound according to Compound (1C) wherein R¹ represents a chlorine atom, Q represents Q8C, R⁷ represents an ethyl group, and R¹³ represents any substituent selected from Group X (hereinafter, referred to as Compound group SX120).

A compound according to Compound (1C) wherein R¹ represents a methyl group, Q represents Q8D, R⁷ represents an ethyl group, and R¹³ represents any substituent selected from Group X (hereinafter, referred to as Compound group SX121).

A compound according to Compound (1C) wherein R¹ represents a methyl group, Q represents Q8E, R⁷ represents an ethyl group, and R¹³ represents any substituent selected from Group X (hereinafter, referred to as Compound group SX122).

A compound according to Compound (1C) wherein R¹ represents a methyl group, Q represents Q8F, R⁷ represents an ethyl group, and R¹³ represents any substituent selected from Group X (hereinafter, referred to as Compound group SX123).

A compound according to Compound (1C) wherein R¹ represents a hydrogen atom, Q represents Q8G, R⁷ represents an ethyl group, and R¹³ represents any substituent selected from Group X (hereinafter, referred to as Compound group SX124).

A compound according to Compound (1C) wherein R¹ represents a hydrogen atom, Q represents Q8H, R⁷ represents an ethyl group, and R¹³ represents any substituent selected from Group X (hereinafter, referred to as Compound group SX125).

A compound according to Compound (1C) wherein R¹ represents a hydrogen atom, Q represents Q8I, R⁷ represents an ethyl group, and R¹³ represents any substituent selected from Group X (hereinafter, referred to as Compound group SX126).

A compound according to Compound (1C) wherein R¹ represents a methyl group, Q represents Q8J, R⁷ represents an ethyl group, and R¹³ represents any substituent selected from Group X (hereinafter, referred to as Compound group SX127).

A compound according to Compound (1C) wherein R¹ represents a chlorine atom, Q represents Q8J, R⁷ represents an ethyl group, and R¹³ represents any substituent selected from Group X (hereinafter, referred to as Compound group SX128).

A compound according to Compound (1C) wherein R¹ represents a methyl group, Q represents Q8K, R⁷ represents an ethyl group, and R¹³ represents any substituent selected from Group X (hereinafter, referred to as Compound group SX129).

A compound according to Compound (1C) wherein R¹ represents a chlorine atom, Q represents Q8K, R⁷ represents an ethyl group, and R¹³ represents any substituent selected from Group X (hereinafter, referred to as Compound group SX130).

A compound according to Compound (1C) wherein R¹ represents a methyl group, Q represents Q8L, R⁷ represents an ethyl group, and R¹³ represents any substituent selected from Group X (hereinafter, referred to as Compound group SX131).

A compound according to Compound (1C) wherein R¹ represents a chlorine atom, Q represents Q8L, R⁷ represents an ethyl group, and R¹³ represents any substituent selected from Group X (hereinafter, referred to as Compound group SX132).

A compound according to Compound (1C) wherein R¹ represents a methyl group, Q represents Q8M, R⁷ represents an ethyl group, and R¹³ represents any substituent selected from Group X (hereinafter, referred to as Compound group SX133).

A compound according to Compound (1C) wherein R¹ represents a methyl group, Q represents Q8N, R⁷ represents an ethyl group, and R¹³ represents any substituent selected from Group X (hereinafter, referred to as Compound group SX134).

A compound according to Compound (1C) wherein R¹ represents a methyl group, Q represents Q8O, R⁷ represents an ethyl group, and R¹³ represents any substituent selected from Group X (hereinafter, referred to as Compound group SX135).

A compound according to Compound (1C) wherein R¹ represents a methyl group, Q represents Q8P, R⁷ represents an ethyl group, and R¹³ represents any substituent selected from Group X (hereinafter, referred to as Compound group SX136).

A compound according to Compound (1C) wherein R¹ represents a methyl group, Q represents Q3, R³ represents a methoxy group, R⁷ represents a hydrogen atom, and R¹³ represents any substituent selected from Group X (hereinafter, referred to as Compound group SX137).

A compound according to Compound (1C) wherein R¹ represents a chlorine atom, Q represents Q3, R³ represents a methoxy group, R⁷ represents a hydrogen atom, and R¹³ represents any substituent selected from Group X (hereinafter, referred to as Compound group SX138).

A compound according to Compound (1C) wherein R¹ represents a methyl group, Q represents Q4, R⁷ represents a hydrogen atom, and R¹³ represents any substituent selected from Group X (hereinafter, referred to as Compound group SX139).

A compound according to Compound (1C) wherein R¹ represents a chlorine atom, Q represents Q4, R⁷ represents a hydrogen atom, and R¹³ represents any substituent selected from Group X (hereinafter, referred to as Compound group SX140).

A compound according to Compound (1C) wherein R¹ represents a methyl group, Q represents Q5, R⁷ represents a hydrogen atom, and R¹³ represents any substituent selected from Group X (hereinafter, referred to as Compound group SX141).

A compound according to Compound (1C) wherein R¹ represents a chlorine atom, Q represents Q5, R⁷ represents a hydrogen atom, and R¹³ represents any substituent selected from Group X (hereinafter, referred to as Compound group SX142).

A compound according to Compound (1C) wherein R¹ represents a methyl group, Q represents Q6, R⁷ represents a hydrogen atom, and R¹³ represents any substituent selected from Group X (hereinafter, referred to as Compound group SX143).

A compound according to Compound (1C) wherein R¹ represents a chlorine atom, Q represents Q6, R⁷ represents a hydrogen atom, and R¹³ represents any substituent selected from Group X (hereinafter, referred to as Compound group SX144).

A compound according to Compound (1C) wherein R¹ represents a methyl group, Q represents Q7, L² represents an oxygen atom, R⁷ represents a hydrogen atom, and R¹³ represents any substituent selected from Group X (hereinafter, referred to as Compound group SX145).

A compound according to Compound (1C) wherein R¹ represents a chlorine atom, Q represents Q7, L² represents an oxygen atom, R⁷ represents a hydrogen atom, and R¹³ represents any substituent selected from Group X (hereinafter, referred to as Compound group SX146).

A compound according to Compound (1C) wherein R¹ represents a methyl group, Q represents Q7, L² represents NH, R⁷ represents a hydrogen atom, and R¹³ represents any substituent selected from Group X (hereinafter, referred to as Compound group SX147).
A compound according to Compound (1C) wherein R¹ represents a chlorine atom, Q represents Q7, L² represents NH, R⁷ represents a hydrogen atom, and R¹³ represents any substituent selected from Group X (hereinafter, referred to as Compound group SX148).

A compound according to Compound (1C) wherein R¹ represents a methyl group, Q represents Q8A, R⁷ represents a hydrogen atom, and R¹³ represents any substituent selected from Group X (hereinafter, referred to as Compound group SX149).

A compound according to Compound (1C) wherein R¹ represents a chlorine atom, Q represents Q8A, R⁷ represents a hydrogen atom, and R¹³ represents any substituent selected from Group X (hereinafter, referred to as Compound group SX150).

A compound according to Compound (1C) wherein R¹ represents a methyl group, Q represents Q8B, R⁷ represents a hydrogen atom, and R¹³ represents any substituent selected from Group X (hereinafter, referred to as Compound group SX151).

A compound according to Compound (1C) wherein R¹ represents a chlorine atom, Q represents Q8B, R⁷ represents a hydrogen atom, and R¹³ represents any substituent selected from Group X (hereinafter, referred to as Compound group SX152).

A compound according to Compound (1C) wherein R¹ represents a methyl group, Q represents Q8C, R⁷ represents a hydrogen atom, and R¹³ represents any substituent selected from Group X (hereinafter, referred to as Compound group SX153).

A compound according to Compound (1C) wherein R¹ represents a chlorine atom, Q represents Q8C, R⁷ represents a hydrogen atom, and R¹³ represents any substituent selected from Group X (hereinafter, referred to as Compound group SX154).

A compound according to Compound (1C) wherein R¹ represents a methyl group, Q represents Q8D, R⁷ represents a hydrogen atom, and R¹³ represents any substituent selected from Group X (hereinafter, referred to as Compound group SX155).

A compound according to Compound (1C) wherein R¹ represents a methyl group, Q represents Q8E, R⁷ represents a hydrogen atom, and R¹³ represents any substituent selected from Group X (hereinafter, referred to as Compound group SX156).

A compound according to Compound (1C) wherein R¹ represents a methyl group, Q represents Q8F, R⁷ represents a hydrogen atom, and R¹³ represents any substituent selected from Group X (hereinafter, referred to as Compound group SX157).

A compound according to Compound (1C) wherein R¹ represents a hydrogen atom, Q represents Q8G, R⁷ represents a hydrogen atom, and R¹³ represents any substituent selected from Group X (hereinafter, referred to as Compound group SX158).

A compound according to Compound (1C) wherein R¹ represents a hydrogen atom, Q represents Q8H, R⁷ represents a hydrogen atom, and R¹³ represents any substituent selected from Group X (hereinafter, referred to as Compound group SX159).

A compound according to Compound (1C) wherein R¹ represents a hydrogen atom, Q represents Q8I, R⁷ represents a hydrogen atom, and R¹³ represents any substituent selected from Group X (hereinafter, referred to as Compound group SX160).

A compound according to Compound (1C) wherein R¹ represents a methyl group, Q represents Q8J, R⁷ represents a hydrogen atom, and R¹³ represents any substituent selected from Group X (hereinafter, referred to as Compound group SX161).

A compound according to Compound (1C) wherein R¹ represents a chlorine atom, Q represents Q8J, R⁷ represents a hydrogen atom, and R¹³ represents any substituent selected from Group X (hereinafter, referred to as Compound group SX162).

A compound according to Compound (1C) wherein R¹ represents a methyl group, Q represents Q8K, R⁷ represents a hydrogen atom, and R¹³ represents any substituent selected from Group X (hereinafter, referred to as Compound group SX163).

A compound according to Compound (1C) wherein R¹ represents a chlorine atom, Q represents Q8K, R⁷ represents a hydrogen atom, and R¹³ represents any substituent selected from Group X (hereinafter, referred to as Compound group SX164).

A compound according to Compound (1C) wherein R¹ represents a methyl group, Q represents Q8L, R⁷ represents a hydrogen atom, and R¹³ represents any substituent selected from Group X (hereinafter, referred to as Compound group SX165).

A compound according to Compound (1C) wherein R¹ represents a chlorine atom, Q represents Q8L, R⁷ represents a hydrogen atom, and R¹³ represents any substituent selected from Group X (hereinafter, referred to as Compound group SX166).

A compound according to Compound (1C) wherein R¹ represents a methyl group, Q represents Q8M, R⁷ represents a hydrogen atom, and R¹³ represents any substituent selected from Group X (hereinafter, referred to as Compound group SX167).

A compound according to Compound (1C) wherein R¹ represents a methyl group, Q represents Q8N, R⁷ represents a hydrogen atom, and R¹³ represents any substituent selected from Group X (hereinafter, referred to as Compound group SX168).

A compound according to Compound (1C) wherein R¹ represents a methyl group, Q represents Q8O, R⁷ represents a hydrogen atom, and R¹³ represents any substituent selected from Group X (hereinafter, referred to as Compound group SX169).

A compound according to Compound (1C) wherein R¹ represents a methyl group, Q represents Q8P, R⁷ represents a hydrogen atom, and R¹³ represents any substituent selected from Group X (hereinafter, referred to as Compound group SX170).

A compound represented by formula (1D) (hereinafter, referred to as Compound (1D)):

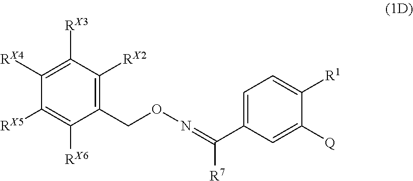

wherein R¹ represents a methyl group, Q represents Q3, R³ represents a methoxy group, R⁷ represents a methyl group, and a combination of R^{X2}, R^{X3}, R^{X4}, R^{X5} and R^{X6} represents any combination described in Combination A (hereinafter, referred to as Compound group SX171).

A compound according to Compound (1D) wherein R¹ represents a chlorine atom, Q represents Q3, R³ represents a methoxy group, R⁷ represents a methyl group, and a combination of R^{X2}, R^{X3}, R^{X4}, R^{X5} and R^{X6} represents any combination described in Combination A (hereinafter, referred to as Compound group SX172).

A compound according to Compound (1D) wherein R¹ represents a methyl group, Q represents Q4, R⁷ represents a methyl group, and a combination of R^{X2}, R^{X3}, R^{X4}, R^{X5} and R^{X6} represents any combination described in Combination A (hereinafter, referred to as Compound group SX173).

A compound according to Compound (1D) wherein R¹ represents a chlorine atom, Q represents Q4, R⁷ represents a methyl group, and a combination of R^{X2}, R^{X3}, R^{X4}, R^{X5} and R^{X6} represents any combination described in Combination A (hereinafter, referred to as Compound group SX174).

A compound according to Compound (1D) wherein R¹ represents a methyl group, Q represents Q5, R⁷ represents a methyl group, and a combination of R^{X2}, R^{X3}, R^{X4}, R^{X5} and R^{X6} represents any combination described in Combination A (hereinafter, referred to as Compound group SX175).

A compound according to Compound (1D) wherein R¹ represents a chlorine atom, Q represents Q5, R⁷ represents a methyl group, and a combination of R^{X2}, R^{X3}, R^{X4}, R^{X5} and R^{X6} represents any combination described in Combination A (hereinafter, referred to as Compound group SX176).

A compound according to Compound (1D) wherein R¹ represents a methyl group, Q represents Q6, R⁷ represents a methyl group, and a combination of R^{X2}, R^{X3}, R^{X4}, R^{X5} and R^{X6} represents any combination described in Combination A (hereinafter, referred to as Compound group SX177).

A compound according to Compound (1D) wherein R¹ represents a chlorine atom, Q represents Q6, R⁷ represents a methyl group, and a combination of R^{X2}, R^{X3}, R^{X4}, R^{X5} and R^{X6} represents any combination described in Combination A (hereinafter, referred to as Compound group SX178).

A compound according to Compound (1D) wherein R¹ represents a methyl group, Q represents Q7, L² represents an oxygen atom, R⁷ represents a methyl group, and a combination of R^{X2}, R^{X3}, R^{X4}, R^{X5} and R^{X6} represents any combination described in Combination A (hereinafter, referred to as Compound group SX179).

A compound according to Compound (1D) wherein R¹ represents a chlorine atom, Q represents Q7, L² represents an oxygen atom, R⁷ represents a methyl group, and a combination of R^{X2}, R^{X3}, R^{X4}, R^{X5} and R^{X6} represents any combination described in Combination A (hereinafter, referred to as Compound group SX180).

A compound according to Compound (1D) wherein R¹ represents a methyl group, Q represents Q7, L² represents NH, R⁷ represents a methyl group, and a combination of R^{X2}, R^{X3}, R^{X4}, R^{X5} and R^{X6} represents any combination described in Combination A (hereinafter, referred to as Compound group SX181).

A compound according to Compound (1D) wherein R¹ represents a chlorine atom, Q represents Q7, L² represents NH, R⁷ represents a methyl group, and a combination of R^{X2}, R^{X3}, R^{X4}, R^{X5} and R^{X6} represents any combination described in Combination A (hereinafter, referred to as Compound group SX182).

A compound according to Compound (1D) wherein R¹ represents a methyl group, Q represents Q8A, R⁷ represents a methyl group, and a combination of R^{X2}, R^{X3}, R^{X4}, R^{X5} and R^{X6} represents any combination described in Combination A (hereinafter, referred to as Compound group SX183).

A compound according to Compound (1D) wherein R¹ represents a chlorine atom, Q represents Q8A, R⁷ represents a methyl group, and a combination of R^{X2}, R^{X3}, R^{X4}, R^{X5} and R^{X6} represents any combination described in Combination A (hereinafter, referred to as Compound group SX184).

A compound according to Compound (1D) wherein R¹ represents a methyl group, Q represents Q8B, R⁷ represents a methyl group, and a combination of R^{X2}, R^{X3}, R^{X4}, R^{X5} and R^{X6} represents any combination described in Combination A (hereinafter, referred to as Compound group SX185).

A compound according to Compound (1D) wherein R¹ represents a chlorine atom, Q represents Q8B, R⁷ represents a methyl group, and a combination of R^{X2}, R^{X3}, R^{X4}, R^{X5} and R^{X6} represents any combination described in Combination A (hereinafter, referred to as Compound group SX186).

A compound according to Compound (1D) wherein R¹ represents a methyl group, Q represents Q8C, R⁷ represents a methyl group, and a combination of R^{X2}, R^{X3}, R^{X4}, R^{X5} and R^{X6} represents any combination described in Combination A (hereinafter, referred to as Compound group SX187).

A compound according to Compound (1D) wherein R¹ represents a chlorine atom, Q represents Q8C, R⁷ represents a methyl group, and a combination of R^{X2}, R^{X3}, R^{X4}, R^{X5} and R^{X6} represents any combination described in Combination A (hereinafter, referred to as Compound group SX188).

A compound according to Compound (1D) wherein R¹ represents a methyl group, Q represents Q8D, R⁷ represents a methyl group, and a combination of R^{X2}, R^{X3}, R^{X4}, R^{X5} and R^{X6} represents any combination described in Combination A (hereinafter, referred to as Compound group SX189).

A compound according to Compound (1D) wherein R¹ represents a methyl group, Q represents Q8E, R⁷ represents a methyl group, and a combination of R^{X2}, R^{X3}, R^{X4}, R^{X5} and R^{X6} represents any combination described in Combination A (hereinafter, referred to as Compound group SX190).

A compound according to Compound (1D) wherein R¹ represents a methyl group, Q represents Q8F, R⁷ represents a methyl group, and a combination of R^{X2}, R^{X3}, R^{X4}, R^{X5} and R^{X6} represents any combination described in Combination A (hereinafter, referred to as Compound group SX191).

A compound according to Compound (1D) wherein R¹ represents a hydrogen atom, Q represents Q8G, R⁷ represents a methyl group, and a combination of R^{X2}, R^{X3}, R^{X4}, R^{X5} and R^{X6} represents any combination described in Combination A (hereinafter, referred to as Compound group SX192).

A compound according to Compound (1D) wherein R¹ represents a hydrogen atom, Q represents Q8H, R⁷ represents a methyl group, and a combination of R^{X2}, R^{X3}, R^{X4}, R^{X5} and R^{X6} represents any combination described in Combination A (hereinafter, referred to as Compound group SX193).

A compound according to Compound (1D) wherein R¹ represents a hydrogen atom, Q represents Q8I, R⁷ represents a methyl group, and a combination of R^{X2}, R^{X3}, R^{X4}, R^{X5} and R^{X6} represents any combination described in Combination A (hereinafter, referred to as Compound group SX194).

A compound according to Compound (1D) wherein R¹ represents a methyl group, Q represents Q8J, R⁷ represents a methyl group, and a combination of R^{X2}, R^{X3}, R^{X4}, R^{X5} and R^{X6} represents any combination described in Combination A (hereinafter, referred to as Compound group SX195).

A compound according to Compound (1D) wherein R¹ represents a chlorine atom, Q represents Q8J, R⁷ represents a methyl group, and a combination of R^{X2}, R^{X3}, R^{X4}, R^{X5} and R^{X6} represents any combination described in Combination A (hereinafter, referred to as Compound group SX196).

A compound according to Compound (1D) wherein R¹ represents a methyl group, Q represents Q8K, R⁷ represents a methyl group, and a combination of R^{X2}, R^{X3}, R^{X4}, R^{X5} and R^{X6} represents any combination described in Combination A (hereinafter, referred to as Compound group SX197).

A compound according to Compound (1D) wherein R¹ represents a chlorine atom, Q represents Q8K, R⁷ represents a methyl group, and a combination of R^{X2}, R^{X3}, R^{X4}, R^{X5} and R^{X6} represents any combination described in Combination A (hereinafter, referred to as Compound group SX198).

A compound according to Compound (1D) wherein R¹ represents a methyl group, Q represents Q8L, R⁷ represents a methyl group, and a combination of R^{X2}, R^{X3}, R^{X4}, R^{X5} and R^{X6} represents any combination described in Combination A (hereinafter, referred to as Compound group SX199).

A compound according to Compound (1D) wherein R¹ represents a chlorine atom, Q represents Q8L, R⁷ represents a methyl group, and a combination of R^{X2}, R^{X3}, R^{X4}, R^{X5} and R^{X6} represents any combination described in Combination A (hereinafter, referred to as Compound group SX200).

A compound according to Compound (1D) wherein R¹ represents a methyl group, Q represents Q8M, R⁷ represents a methyl group, and a combination of R^{X2}, R^{X3}, R^{X4}, R^{X5} and R^{X6} represents any combination described in Combination A (hereinafter, referred to as Compound group SX201).

A compound according to Compound (1D) wherein R¹ represents a methyl group, Q represents Q8N, R⁷ represents a methyl group, and a combination of R^{X2}, R^{X3}, R^{X4}, R^{X5} and R^{X6} represents any combination described in Combination A (hereinafter, referred to as Compound group SX202).

A compound according to Compound (1D) wherein R¹ represents a methyl group, Q represents Q8O, R⁷ represents a methyl group, and a combination of R^{X2}, R^{X3}, R^{X4}, R^{X5} and R^{X6} represents any combination described in Combination A (hereinafter, referred to as Compound group SX203).

A compound according to Compound (1D) wherein R¹ represents a methyl group, Q represents Q8P, R⁷ represents a methyl group, and a combination of R^{X2}, R^{X3}, R^{X4}, R^{X5} and R^{X6} represents any combination described in Combination A (hereinafter, referred to as Compound group SX204).

A compound according to Compound (1D) wherein R¹ represents a methyl group, Q represents Q3, R³ represents a methoxy group, R⁷ represents an ethyl group, and a combination of R^{X2}, R^{X3}, R^{X4}, R^{X5} and R^{X6} represents any combination described in Combination A (hereinafter, referred to as Compound group SX205).

A compound according to Compound (1D) wherein R¹ represents a chlorine atom, Q represents Q3, R³ represents a methoxy group, R⁷ represents an ethyl group, and a combination of R^{X2}, R^{X3}, R^{X4}, R^{X5} and R^{X6} represents any combination described in Combination A (hereinafter, referred to as Compound group SX206).

A compound according to Compound (1D) wherein R¹ represents a methyl group, Q represents Q4, R⁷ represents an ethyl group, and a combination of R^{X2}, R^{X3}, R^{X4}, R^{X5} and R^{X6} represents any combination described in Combination A (hereinafter, referred to as Compound group SX207).

A compound according to Compound (1D) wherein R¹ represents a chlorine atom, Q represents Q4, R⁷ represents an ethyl group, and a combination of R^{X2}, R^{X3}, R^{X4}, R^{X5} and R^{X6} represents any combination described in Combination A (hereinafter, referred to as Compound group SX208).

A compound according to Compound (1D) wherein R¹ represents a methyl group, Q represents Q5, R⁷ represents an ethyl group, and a combination of R^{X2}, R^{X3}, R^{X4}, R^{X5} and R^{X6} represents any combination described in Combination A (hereinafter, referred to as Compound group SX209).

A compound according to Compound (1D) wherein R¹ represents a chlorine atom, Q represents Q5, R⁷ represents an ethyl group, and a combination of R^{X2}, R^{X3}, R^{X4}, R^{X5} and R^{X6} represents any combination described in Combination A (hereinafter, referred to as Compound group SX210).

A compound according to Compound (1D) wherein R¹ represents a methyl group, Q represents Q6, R⁷ represents an ethyl group, and a combination of R^{X2}, R^{X3}, R^{X4}, R^{X5} and R^{X6} represents any combination described in Combination A (hereinafter, referred to as Compound group SX211).

A compound according to Compound (1D) wherein R¹ represents a chlorine atom, Q represents Q6, R⁷ represents an ethyl group, and a combination of R^{X2}, R^{X3}, R^{X4}, R^{X5} and R^{X6} represents any combination described in Combination A (hereinafter, referred to as Compound group SX212).

A compound according to Compound (1D) wherein R¹ represents a methyl group, Q represents Q7, L² represents an oxygen atom, R⁷ represents an ethyl group, and a combination of R^{X2}, R^{X3}, R^{X4}, R^{X5} and R^{X6} represents any combination described in Combination A (hereinafter, referred to as Compound group SX213).

A compound according to Compound (1D) wherein R¹ represents a chlorine atom, Q represents Q7, L² represents an oxygen atom, R⁷ represents an ethyl group, and a combination of R^{X2}, R^{X3}, R^{X4}, R^{X5} and R^{X6} represents any combination described in Combination A (hereinafter, referred to as Compound group SX214).

A compound according to Compound (1D) wherein R¹ represents a methyl group, Q represents Q7, L² represents NH, R⁷ represents an ethyl group, and a combination of R^{X2}, R^{X3}, R^{X4}, R^{X5} and R^{X6} represents any combination described in Combination A (hereinafter, referred to as Compound group SX215).

A compound according to Compound (1D) wherein R¹ represents a chlorine atom, Q represents Q7, L² represents NH, R⁷ represents an ethyl group, and a combination of R^{X2}, R^{X3}, R^{X4}, R^{X5} and R^{X6} represents any combination described in Combination A (hereinafter, referred to as Compound group SX216).

A compound according to Compound (1D) wherein R¹ represents a methyl group, Q represents Q8A, R⁷ represents an ethyl group, and a combination of R^{X2}, R^{X3}, R^{X4}, R^{X5} and R^{X6} represents any combination described in Combination A (hereinafter, referred to as Compound group SX217).

A compound according to Compound (1D) wherein R¹ represents a chlorine atom, Q represents Q8A, R⁷ represents an ethyl group, and a combination of R^{X2}, R^{X3}, R^{X4}, R^{X5} and R^{X6} represents any combination described in Combination A (hereinafter, referred to as Compound group SX218).

A compound according to Compound (1D) wherein R¹ represents a methyl group, Q represents Q8B, R⁷ represents an ethyl group, and a combination of R^{X2}, R^{X3}, R^{X4}, R^{X5} and R^{X6} represents any combination described in Combination A (hereinafter, referred to as Compound group SX219).

A compound according to Compound (1D) wherein R¹ represents a chlorine atom, Q represents Q8B, R⁷ represents an ethyl group, and a combination of R^{X2}, R^{X3}, R^{X4}, R^{X5} and R^{X6} represents any combination described in Combination A (hereinafter, referred to as Compound group SX220).

A compound according to Compound (1D) wherein R¹ represents a methyl group, Q represents Q8C, R⁷ represents an ethyl group, and a combination of R^{X2}, R^{X3}, R^{X4}, R^{X5} and R^{X6} represents any combination described in Combination A (hereinafter, referred to as Compound group SX221).

A compound according to Compound (1D) wherein R¹ represents a chlorine atom, Q represents Q8C, R⁷ represents an ethyl group, and a combination of R^{X2}, R^{X3}, R^{X4}, R^{X5} and R^{X6} represents any combination described in Combination A (hereinafter, referred to as Compound group SX222).

A compound according to Compound (1D) wherein R¹ represents a methyl group, Q represents Q8D, R⁷ represents an ethyl group, and a combination of R^{X2}, R^{X3}, R^{X4}, R^{X5} and R^{X6} represents any combination described in Combination A (hereinafter, referred to as Compound group SX223).

A compound according to Compound (1D) wherein R¹ represents a methyl group, Q represents Q8E, R⁷ represents an ethyl group, and a combination of R^{X2}, R^{X3}, R^{X4}, R^{X5} and R^{X6} represents any combination described in Combination A (hereinafter, referred to as Compound group SX224).

A compound according to Compound (1D) wherein R¹ represents a methyl group, Q represents Q8F, R⁷ represents an ethyl group, and a combination of R^{X2}, R^{X3}, R^{X4}, R^{X5} and R^{X6} represents any combination described in Combination A (hereinafter, referred to as Compound group SX225).

A compound according to Compound (1D) wherein R¹ represents a hydrogen atom, Q represents Q8G, R⁷ represents an ethyl group, and a combination of R^{X2}, R^{X3}, R^{X4}, R^{X5} and R^{X6} represents any combination described in Combination A (hereinafter, referred to as Compound group SX226).

A compound according to Compound (1D) wherein R¹ represents a hydrogen atom, Q represents Q8H, R⁷ represents an ethyl group, and a combination of R^{X2}, R^{X3}, R^{X4}, R^{X5} and R^{X6} represents any combination described in Combination A (hereinafter, referred to as Compound group SX227).

A compound according to Compound (1D) wherein R¹ represents a hydrogen atom, Q represents Q8I, R⁷ represents an ethyl group, and a combination of R^{X2}, R^{X3}, R^{X4}, R^{X5} and R^{X6} represents any combination described in Combination A (hereinafter, referred to as Compound group SX228).

A compound according to Compound (1D) wherein R¹ represents a methyl group, Q represents Q8J, R⁷ represents an ethyl group, and a combination of R^{X2}, R^{X3}, R^{X4}, R^{X5} and R^{X6} represents any combination described in Combination A (hereinafter, referred to as Compound group SX229).

A compound according to Compound (1D) wherein R¹ represents a chlorine atom, Q represents Q8J, R⁷ represents an ethyl group, and a combination of R^{X2}, R^{X3}, R^{X4}, R^{X5} and R^{X6} represents any combination described in Combination A (hereinafter, referred to as Compound group SX230).

A compound according to Compound (1D) wherein R¹ represents a methyl group, Q represents Q8K, R⁷ represents an ethyl group, and a combination of R^{X2}, R^{X3}, R^{X4}, R^{X5} and R^{X6} represents any combination described in Combination A (hereinafter, referred to as Compound group SX231).

A compound according to Compound (1D) wherein R¹ represents a chlorine atom, Q represents Q8K, R⁷ represents an ethyl group, and a combination of R^{X2}, R^{X3}, R^{X4}, R^{X5} and R^{X6} represents any combination described in Combination A (hereinafter, referred to as Compound group SX232).

A compound according to Compound (1D) wherein R¹ represents a methyl group, Q represents Q8L, R⁷ represents an ethyl group, and a combination of R^{X2}, R^{X3}, R^{X4}, R^{X5} and R^{X6} represents any combination described in Combination A (hereinafter, referred to as Compound group SX233).

A compound according to Compound (1D) wherein R¹ represents a chlorine atom, Q represents Q8L, R⁷ represents an ethyl group, and a combination of R^{X2}, R^{X3}, R^{X4}, R^{X5} and R^{X6} represents any combination described in Combination A (hereinafter, referred to as Compound group SX234).

A compound according to Compound (1D) wherein R¹ represents a methyl group, Q represents Q8M, R⁷ represents an ethyl group, and a combination of R^{X2}, R^{X3}, R^{X4}, R^{X5} and R^{X6} represents any combination described in Combination A (hereinafter, referred to as Compound group SX235).

A compound according to Compound (1D) wherein R¹ represents a methyl group, Q represents Q8N, R⁷ represents an ethyl group, and a combination of R^{X2}, R^{X3}, R^{X4}, R^{X5} and R^{X6} represents any combination described in Combination A (hereinafter, referred to as Compound group SX236).

A compound according to Compound (1D) wherein R¹ represents a methyl group, Q represents Q8O, R⁷ represents an ethyl group, and a combination of R^{X2}, R^{X3}, R^{X4}, R^{X5} and R^{X6} represents any combination described in Combination A (hereinafter, referred to as Compound group SX237).

A compound according to Compound (1D) wherein R¹ represents a methyl group, Q represents Q8P, R⁷ represents an ethyl group, and a combination of R^{X2}, R^{X3}, R^{X4}, R^{X5} and R^{X6} represents any combination described in Combination A (hereinafter, referred to as Compound group SX238).

A compound according to Compound (1D) wherein R¹ represents a methyl group, Q represents Q3, R³ represents a methoxy group, R⁷ represents a hydrogen atom, and a combination of R^{X2}, R^{X3}, R^{X4}, R^{X5} and R^{X6} represents any combination described in Combination A (hereinafter, referred to as Compound group SX239).

A compound according to Compound (1D) wherein R¹ represents a chlorine atom, Q represents Q3, R³ represents a methoxy group, R⁷ represents a hydrogen atom, and a combination of R^{X2}, R^{X3}, R^{X4}, R^{X5} and R^{X6} represents any combination described in Combination A (hereinafter, referred to as Compound group SX240).

A compound according to Compound (1D) wherein R¹ represents a methyl group, Q represents Q4, R⁷ represents a hydrogen atom, and a combination of R^{X2}, R^{X3}, R^{X4}, R^{X5} and R^{X6} represents any combination described in Combination A (hereinafter, referred to as Compound group SX241).

A compound according to Compound (1D) wherein R¹ represents a chlorine atom, Q represents Q4, R⁷ represents a hydrogen atom, and a combination of R^{X2}, R^{X3}, R^{X4}, R^{X5} and R^{X6} represents any combination described in Combination A (hereinafter, referred to as Compound group SX242).

A compound according to Compound (1D) wherein R¹ represents a methyl group, Q represents Q5, R⁷ represents a hydrogen atom, and a combination of R^{X2}, R^{X3}, R^{X4}, R^{X5} and R^{X6} represents any combination described in Combination A (hereinafter, referred to as Compound group SX243).

A compound according to Compound (1D) wherein R¹ represents a chlorine atom, Q represents Q5, R⁷ represents a hydrogen atom, and a combination of R^{X2}, R^{X3}, R^{X4}, R^{X5} and R^{X6} represents any combination described in Combination A (hereinafter, referred to as Compound group SX244).

A compound according to Compound (1D) wherein R¹ represents a methyl group, Q represents Q6, R⁷ represents a hydrogen atom, and a combination of R^{X2}, R^{X3}, R^{X4}, R^{X5} and R^{X6} represents any combination described in Combination A (hereinafter, referred to as Compound group SX245).

A compound according to Compound (1D) wherein R¹ represents a chlorine atom, Q represents Q6, R⁷ represents a hydrogen atom, and a combination of R^{X2}, R^{X3}, R^{X4}, R^{X5} and R^{X6} represents any combination described in Combination A (hereinafter, referred to as Compound group SX246).

A compound according to Compound (1D) wherein R¹ represents a methyl group, Q represents Q7, L² represents an oxygen atom, R⁷ represents a hydrogen atom, and a combination of R^{X2}, R^{X3}, R^{X4}, R^{X5} and R^{X6} represents any combination described in Combination A (hereinafter, referred to as Compound group SX247).

A compound according to Compound (1D) wherein R¹ represents a chlorine atom, Q represents Q7, L² represents an oxygen atom, R⁷ represents a hydrogen atom, and a combination of R^{X2}, R^{X3}, R^{X4}, R^{X5} and R^{X6} represents any combination described in Combination A (hereinafter, referred to as Compound group SX248).

A compound according to Compound (1D) wherein R¹ represents a methyl group, Q represents Q7, L² represents NH, R⁷ represents a hydrogen atom, and a combination of R^{X2}, R^{X3}, R^{X4}, R^{X5} and R^{X6} represents any combination described in Combination A (hereinafter, referred to as Compound group SX249).

A compound according to Compound (1D) wherein R¹ represents a chlorine atom, Q represents Q7, L² represents NH, R⁷ represents a hydrogen atom, and a combination of R^{X2}, R^{X3}, R^{X4}, R^{X5} and R^{X6} represents any combination described in Combination A (hereinafter, referred to as Compound group SX250).

A compound according to Compound (1D) wherein R¹ represents a methyl group, Q represents Q8A, R⁷ represents a hydrogen atom, and a combination of R^{X2}, R^{X3}, R^{X4}, R^{X5} and R^{X6} (hereinafter, referred to as Compound group SX251).

A compound according to Compound (1D) wherein R¹ represents a chlorine atom, Q represents Q8A, R⁷ represents a hydrogen atom, and a combination of R^{X2}, R^{X3}, R^{X4}, R^{X5} and R^{X6} represents any combination described in Combination A (hereinafter, referred to as Compound group SX252).

A compound according to Compound (1D) wherein R¹ represents a methyl group, Q represents Q8B, R⁷ represents a hydrogen atom, and a combination of R^{X2}, R^{X3}, R^{X4}, R^{X5} and R^{X6} represents any combination described in Combination A (hereinafter, referred to as Compound group SX253).

A compound according to Compound (1D) wherein R¹ represents a chlorine atom, Q represents Q8B, R⁷ represents a hydrogen atom, and a combination of R^{X2}, R^{X3}, R^{X4}, R^{X5} and R^{X6} represents any combination described in Combination A (hereinafter, referred to as Compound group SX254).

A compound according to Compound (1D) wherein R¹ represents a methyl group, Q represents Q8C, R⁷ represents a hydrogen atom, and a combination of R^{X2}, R^{X3}, R^{X4}, R^{X5} and R^{X6} represents any combination described in Combination A (hereinafter, referred to as Compound group SX255).

A compound according to Compound (1D) wherein R¹ represents a chlorine atom, Q represents Q8C, R⁷ represents a hydrogen atom, and a combination of R^{X2}, R^{X3}, R^{X4}, R^{X5} and R^{X6} represents any combination described in Combination A (hereinafter, referred to as Compound group SX256).

A compound according to Compound (1D) wherein R¹ represents a methyl group, Q represents Q8D, R⁷ represents a hydrogen atom, and a combination of R^{X2}, R^{X3}, R^{X4}, R^{X5} and R^{X6} represents any combination described in Combination A (hereinafter, referred to as Compound group SX257).

A compound according to Compound (1D) wherein R¹ represents a methyl group, Q represents Q8E, R⁷ represents a hydrogen atom, and a combination of R^{X2}, R^{X3}, R^{X4}, R^{X5} and R^{X6} represents any combination described in Combination A (hereinafter, referred to as Compound group SX258).

A compound according to Compound (1D) wherein R¹ represents a methyl group, Q represents Q8F, R⁷ represents a hydrogen atom, and a combination of R^{X2}, R^{X3}, R^{X4}, R^{X5} and R^{X6} represents any combination described in Combination A (hereinafter, referred to as Compound group SX259).

A compound according to Compound (1D) wherein R¹ represents a hydrogen atom, Q represents Q8G, R⁷ represents a hydrogen atom, and a combination of R^{X2}, R^{X3}, R^{X4}, R^{X5} and R^{X6} represents any combination described in Combination A (hereinafter, referred to as Compound group SX260).

A compound according to Compound (1D) wherein R¹ represents a hydrogen atom, Q represents Q8H, R⁷ represents a hydrogen atom, and a combination of R^{X2}, R^{X3}, R^{X4}, R^{X5} and R^{X6} represents any combination described in Combination A (hereinafter, referred to as Compound group SX261).

A compound according to Compound (1D) wherein R¹ represents a hydrogen atom, Q represents Q8I, R⁷ represents a hydrogen atom, and a combination of R^{X2}, R^{X3}, R^{X4}, R^{X5} and R^{X6} represents any combination described in Combination A (hereinafter, referred to as Compound group SX262).

A compound according to Compound (1D) wherein R¹ represents a methyl group, Q represents Q8J, R⁷ represents a hydrogen atom, and a combination of R^{X2}, R^{X3}, R^{X4}, R^{X5} and R^{X6} represents any combination described in Combination A (hereinafter, referred to as Compound group SX263).

A compound according to Compound (1D) wherein R¹ represents a chlorine atom, Q represents Q8J, R⁷ represents a hydrogen atom, and a combination of R^{X2}, R^{X3}, R^{X4}, R^{X5} and R^{X6} represents any combination described in Combination A (hereinafter, referred to as Compound group SX264).

A compound according to Compound (1D) wherein R¹ represents a methyl group, Q represents Q8K, R⁷ represents a hydrogen atom, and a combination of R^{X2}, R^{X3}, R^{X4}, R^{X5} and R^{X6} represents any combination described in Combination A (hereinafter, referred to as Compound group SX265).

A compound according to Compound (1D) wherein R¹ represents a chlorine atom, Q represents Q8K, R⁷ represents a hydrogen atom, and a combination of R^{X2}, R^{X3}, R^{X4}, R^{X5} and R^{X6} represents any combination described in Combination A (hereinafter, referred to as Compound group SX266).

A compound according to Compound (1D) wherein R¹ represents a methyl group, Q represents Q8L, R⁷ represents a hydrogen atom, and a combination of R^{X2}, R^{X3}, R^{X4}, R^{X5} and R^{X6} represents any combination described in Combination A (hereinafter, referred to as Compound group SX267).

A compound according to Compound (1D) wherein R¹ represents a chlorine atom, Q represents Q8L, R⁷ represents a hydrogen atom, and a combination of R^{X2}, R^{X3}, R^{X4}, R^{X5} and R^{X6} represents any combination described in Combination A (hereinafter, referred to as Compound group SX268).

A compound according to Compound (1D) wherein R¹ represents a methyl group, Q represents Q8M, R⁷ represents a hydrogen atom, and a combination of R^{X2}, R^{X3}, R^{X4}, R^{X5} and R^{X6} represents any combination described in Combination A (hereinafter, referred to as Compound group SX269).

A compound according to Compound (1D) wherein R¹ represents a methyl group, Q represents Q8N, R⁷ represents a hydrogen atom, and a combination of R^{X2}, R^{X3}, R^{X4}, R^{X5} and R^{X6} represents any combination described in Combination A (hereinafter, referred to as Compound group SX270).

A compound according to Compound (1D) wherein R¹ represents a methyl group, Q represents Q8O, R⁷ represents a hydrogen atom, and a combination of R^{X2}, R^{X3}, R^{X4}, R^{X5} and R^{X6} (hereinafter, referred to as Compound group SX271).

A compound according to Compound (1D) wherein R¹ represents a methyl group, Q represents Q8P, R⁷ represents a hydrogen atom, and a combination of R^{X2}, R^{X3}, R^{X4}, R^{X5} and R^{X6} represents any combination described in Combination A (hereinafter, referred to as Compound group SX272).

A compound represented by formula (1G) (hereinafter, referred to as Compound (1G)):

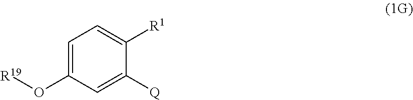

wherein R¹ represents a methyl group, Q represents Q3, R³ represents a methoxy group, and R¹⁹ represents any substituent selected from Group Y (hereinafter, referred to as Compound group SX273).
Group Y: a group consisting of Et, Pr, i-Pr, (CH₂)₃CH₃, CH₂CH(CH₃)₂, CH(CH₃)CH₂CH₃, t-Bu, CH₂c-Pr, (CH₂)₄CH₃, (CH₂)₂CH(CH₃)₂, CH(CH₃)(CH₂)₂CH₃, CH(CH₂CH₃)CH₂CH₃, CH₂CH(CH₃)CH₂CH₃, C(CH₃)₂CH₂CH₃, CH₂t-Bu, CH₂c-Bu, CH₂c-Pen, (CH₂)₂c-Pr, (1-methylcyclopropyl)methyl, (2-methylcyclopropyl)methyl, 1-cyclopropylethyl, (CH₂)₃c-Pr, (CH₂)₅CH₃, (CH₂)₃CH(CH₃)₂, (CH₂)₂CH(CH₃)CH₂CH₃, CH₂CH(CH₃)(CH₂)₂CH₃, CH(CH₃)(CH₂)₃CH₃, CH₂CH(CH₂CH₃)₂, heptyl, CH₂CH═CH₂, CH₂CH═CHCH₃, CH₂CH═C(CH₃)₂, CH₂CH═CF₂, CH₂CH═CCl₂, CH₂CH═CHCH₂CH₃, CH₂CH═CH(CH₂)₂CH₃, CH₂C(CH₃)═CH₂, CH₂C(CH₃)═CHCH₃, CH₂C(CH₃)═C(CH₃)₂, CH₂C(CH₃)═CHCH₂CH₃, CH₂CF═CH₂, CH₂CF═CHCH₃, CH₂CF═C(CH₃)₂, CH₂CF═CF₂, CH₂CF═CHCH₂CH₃, CH₂CF═CH(CH₂)₂CH₃, CH₂CCl═CH₂, CH₂CCl═CHCH₃, CH₂CCl═C(CH₃)₂, CH₂CCl═CCl₂, CH₂CCl═CHCH₂CH₃, CH₂CCl═CH(CH₂)₂CH₃, (CH₂)₂CH═CH₂, (CH₂)₂CH═CHCH₃, (CH₂)₂CH═CHCH₂CH₃, (CH₂)₂CH═C(CH₃)₂, (CH₂)₂C(CH₃)═CH₂, (CH₂)₂C(CH₃)═CHCH₃, (CH₂)₃CH═CH₂, (CH₂)₃C(CH₃)═CH₂, (CH₂)₄CH═CH₂, CH₂C≡CH, CH₂C≡CCH₃, CH₂C≡CCH₂CH₃, CH₂C≡Cc-Pr, (CH₂)₂C≡CH, (CH₂)₂C≡CCH₃, (CH₂)₂C≡CCH₂CH₃, (CH₂)₃C═CH, (CH₂)₃C═CCH₃, CH₂Cl, CH₂Br, CH₂OCH₃, CH₂OCH₂CH₃, CH₂O(CH₂)₂CH₃, CHF₂, CF₃, CH₂CF₃, CH₂CHF₂, CH₂CH₂CHF₂, CH₂CH₂CF₃, CH₂CF₂CF₃, (CH₂)₂CF₂CF₃, CH₂(CF₂)₂CF₃, (CH₂)₂CF(CF₃)₂, (CH₂)₂(CF₂)₅CF₃, CF₃, CF₂CHF(CF₃), CF₂CHF(OCF₃), CH₂CF₂CF₂H, c-Pr, and c-Bu.

A compound according to Compound (1G) wherein R¹ represents a chlorine atom, Q represents Q3, R³ represents a methoxy group, and R¹⁹ represents any substituent selected from Group Y (hereinafter, referred to as Compound group SX274).

A compound according to Compound (1G) wherein R¹ represents a methyl group, Q represents Q4, and R¹⁹ represents any substituent selected from Group Y (hereinafter, referred to as Compound group SX275).

A compound according to Compound (1G) wherein R¹ represents a chlorine atom, Q represents Q4, and R¹⁹ represents any substituent selected from Group Y (hereinafter, referred to as Compound group SX276).

A compound according to Compound (1G) wherein R¹ represents a methyl group, Q represents Q5, and R¹⁹ represents any substituent selected from Group Y (hereinafter, referred to as Compound group SX277).

A compound according to Compound (1G) wherein R¹ represents a chlorine atom, Q represents Q5, and R¹⁹ represents any substituent selected from Group Y (hereinafter, referred to as Compound group SX278).

A compound according to Compound (1G) wherein R¹ represents a methyl group, Q represents Q6, and R¹⁹ represents any substituent selected from Group Y (hereinafter, referred to as Compound group SX279).

A compound according to Compound (1G) wherein R¹ represents a chlorine atom, Q represents Q6, and R¹⁹ represents any substituent selected from Group Y (hereinafter, referred to as Compound group SX280).

A compound according to Compound (1G) wherein R¹ represents a methyl group, Q represents Q7, L² represents an oxygen atom, and R¹⁹ represents any substituent selected from Group Y (hereinafter, referred to as Compound group SX281).

A compound according to Compound (1G) wherein R¹ represents a chlorine atom, Q represents Q7, L² represents an oxygen atom, and R¹⁹ represents any substituent selected from Group Y (hereinafter, referred to as Compound group SX282).

A compound according to Compound (1G) wherein R¹ represents a methyl group, Q represents Q7, L² represents NH, and R¹⁹ represents any substituent selected from Group Y (hereinafter, referred to as Compound group SX283).

A compound according to Compound (1G) wherein R¹ represents a chlorine atom, Q represents Q7, L² represents NH, and R¹⁹ represents any substituent selected from Group Y (hereinafter, referred to as Compound group SX284).

A compound according to Compound (1G) wherein R¹ represents a methyl group, Q represents Q8A, and R¹⁹ represents any substituent selected from Group Y (hereinafter, referred to as Compound group SX285).

A compound according to Compound (1G) wherein R¹ represents a chlorine atom, Q represents Q8A, and R¹⁹ represents any substituent selected from Group Y (hereinafter, referred to as Compound group SX286).

A compound according to Compound (1G) wherein R¹ represents a methyl group, Q represents Q8B, and R¹⁹ represents any substituent selected from Group Y (hereinafter, referred to as Compound group SX287).

A compound according to Compound (1G) wherein R¹ represents a chlorine atom, Q represents Q8B, and R¹⁹ represents any substituent selected from Group Y (hereinafter, referred to as Compound group SX288).

A compound according to Compound (1G) wherein R¹ represents a methyl group, Q represents Q8C, and R¹⁹ represents any substituent selected from Group Y (hereinafter, referred to as Compound group SX289).

A compound according to Compound (1G) wherein R¹ represents a chlorine atom, Q represents Q8C, and R¹⁹ represents any substituent selected from Group Y (hereinafter, referred to as Compound group SX290).

A compound according to Compound (1G) wherein R¹ represents a methyl group, Q represents Q8D, and R¹⁹ represents any substituent selected from Group Y (hereinafter, referred to as Compound group SX291).

A compound according to Compound (1G) wherein R¹ represents a methyl group, Q represents Q8E, and R¹⁹ represents any substituent selected from Group Y (hereinafter, referred to as Compound group SX292).

A compound according to Compound (1G) wherein R¹ represents a methyl group, Q represents Q8F, and R¹⁹ represents any substituent selected from Group Y (hereinafter, referred to as Compound group SX293).

A compound according to Compound (1G) wherein R¹ represents a hydrogen atom, Q represents Q8G, and R¹⁹ represents any substituent selected from Group Y (hereinafter, referred to as Compound group SX294).

A compound according to Compound (1G) wherein R¹ represents a hydrogen atom, Q represents Q8H, and R¹⁹ represents any substituent selected from Group Y (hereinafter, referred to as Compound group SX295).

A compound according to Compound (1G) wherein R¹ represents a hydrogen atom, Q represents Q8I, and R¹⁹ represents any substituent selected from Group Y (hereinafter, referred to as Compound group SX296).

A compound according to Compound (1G) wherein R¹ represents a methyl group, Q represents Q8J, and R¹⁹ represents any substituent selected from Group Y (hereinafter, referred to as Compound group SX297).

A compound according to Compound (1G) wherein R¹ represents a chlorine atom, Q represents Q8J, and R¹⁹ represents any substituent selected from Group Y (hereinafter, referred to as Compound group SX298).

A compound according to Compound (1G) wherein R¹ represents a methyl group, Q represents Q8K, and R¹⁹ represents any substituent selected from Group Y (hereinafter, referred to as Compound group SX299).

A compound according to Compound (1G) wherein R¹ represents a chlorine atom, Q represents Q8K, and R¹⁹ represents any substituent selected from Group Y (hereinafter, referred to as Compound group SX300).

A compound according to Compound (1G) wherein R¹ represents a methyl group, Q represents Q8L, and R¹⁹ represents any substituent selected from Group Y (hereinafter, referred to as Compound group SX301).

A compound according to Compound (1G) wherein R¹ represents a chlorine atom, Q represents Q8L, and R¹⁹ represents any substituent selected from Group Y (hereinafter, referred to as Compound group SX302).

A compound according to Compound (1G) wherein R¹ represents a methyl group, Q represents Q8M, and R¹⁹ represents any substituent selected from Group Y (hereinafter, referred to as Compound group SX303).

A compound according to Compound (1G) wherein R¹ represents a methyl group, Q represents Q8N, and R¹⁹ represents any substituent selected from Group Y (hereinafter, referred to as Compound group SX304).

A compound according to Compound (1G) wherein R¹ represents a methyl group, Q represents Q8O, and R¹⁹ represents any substituent selected from Group Y (hereinafter, referred to as Compound group SX305).

A compound represented by formula (1H) (hereinafter, referred to as Compound (1H)):

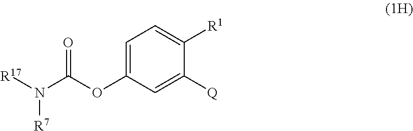

wherein R¹ represents a methyl group, Q represents Q3, R³ represents a methoxy group, R⁷ represents a methyl group, and R¹⁷ represents any substituent selected from Group X (hereinafter, referred to as Compound group SX306).

A compound according to Compound (1H) wherein R¹ represents a chlorine atom, Q represents Q3, R³ represents a methoxy group, R⁷ represents a methyl group, and R¹⁷ represents any substituent selected from Group X (hereinafter, referred to as Compound group SX307).

A compound according to Compound (1H) wherein R¹ represents a methyl group, Q represents Q3, R³ represents a methoxy group, R⁷ represents an ethyl group, and R¹⁷ represents any substituent selected from Group X (hereinafter, referred to as Compound group SX308).

A compound according to Compound (1H) wherein R¹ represents a chlorine atom, Q represents Q3, R³ represents a methoxy group, R⁷ represents an ethyl group, and R¹⁷ represents any substituent selected from Group X (hereinafter, referred to as Compound group SX309).

A compound according to Compound (1H) wherein R¹ represents a methyl group, Q represents Q4, R⁷ represents a methyl group, and R¹⁷ represents any substituent selected from Group X (hereinafter, referred to as Compound group SX310).

A compound according to Compound (1H) wherein R¹ represents a chlorine atom, Q represents Q4, R⁷ represents a methyl group, and R¹⁷ represents any substituent selected from Group X (hereinafter, referred to as Compound group SX311).

A compound according to Compound (1H) wherein R¹ represents a methyl group, Q represents Q4, R⁷ represents an ethyl group, and R¹⁷ represents any substituent selected from Group X (hereinafter, referred to as Compound group SX312).

A compound according to Compound (1H) wherein R¹ represents a chlorine atom, Q represents Q4, R⁷ represents an ethyl group, and R¹⁷ represents any substituent selected from Group X (hereinafter, referred to as Compound group SX313).

A compound according to Compound (1H) wherein R¹ represents a methyl group, Q represents Q5, R⁷ represents a methyl group, and R¹⁷ represents any substituent selected from Group X (hereinafter, referred to as Compound group SX314).

A compound according to Compound (1H) wherein R¹ represents a chlorine atom, Q represents Q5, R⁷ represents a methyl group, and R¹⁷ represents any substituent selected from Group X (hereinafter, referred to as Compound group SX315).

A compound according to Compound (1H) wherein R¹ represents a methyl group, Q represents Q5, R⁷ represents an ethyl group, and R¹⁷ represents any substituent selected from Group X (hereinafter, referred to as Compound group SX316).

A compound according to Compound (1H) wherein R¹ represents a chlorine atom, Q represents Q5, R⁷ represents an ethyl group, and R¹⁷ represents any substituent selected from Group X (hereinafter, referred to as Compound group SX317).

A compound according to Compound (1H) wherein R¹ represents a methyl group, Q represents Q6, R⁷ represents a methyl group, and R¹⁷ represents any substituent selected from Group X (hereinafter, referred to as Compound group SX318).

A compound according to Compound (1H) wherein R¹ represents a chlorine atom, Q represents Q6, R⁷ represents a methyl group, and R¹⁷ represents any substituent selected from Group X (hereinafter, referred to as Compound group SX319).

A compound according to Compound (1H) wherein R¹ represents a methyl group, Q represents Q6, R⁷ represents an ethyl group, and R¹⁷ represents any substituent selected from Group X (hereinafter, referred to as Compound group SX320).

A compound according to Compound (1H) wherein R¹ represents a chlorine atom, Q represents Q6, R⁷ represents an ethyl group, and R¹⁷ represents any substituent selected from Group X (hereinafter, referred to as Compound group SX321).

A compound according to Compound (1H) wherein R¹ represents a methyl group, Q represents Q7, L² represents an oxygen atom, R⁷ represents a methyl group, and R¹⁷ represents any substituent selected from Group X (hereinafter, referred to as Compound group SX322).

A compound according to Compound (1H) wherein R¹ represents a chlorine atom, Q represents Q7, L² represents an oxygen atom, R⁷ represents a methyl group, and R¹⁷ represents any substituent selected from Group X (hereinafter, referred to as Compound group SX323).

A compound according to Compound (1H) wherein R¹ represents a methyl group, Q represents Q7, L² represents an oxygen atom, R⁷ represents an ethyl group, and R¹⁷ represents any substituent selected from Group X (hereinafter, referred to as Compound group SX324).

A compound according to Compound (1H) wherein R¹ represents a chlorine atom, Q represents Q7, L² represents an oxygen atom, R⁷ represents an ethyl group, and R¹⁷ represents any substituent selected from Group X (hereinafter, referred to as Compound group SX325).

A compound according to Compound (1H) wherein R¹ represents a methyl group, Q represents Q7, L² represents NH, R⁷ represents a methyl group, and R¹⁷ represents any substituent selected from Group X (hereinafter, referred to as Compound group SX326).

A compound according to Compound (1H) wherein R¹ represents a chlorine atom, Q represents Q7, L² represents NH, R⁷ represents a methyl group, and R¹⁷ represents any substituent selected from Group X (hereinafter, referred to as Compound group SX327).

A compound according to Compound (1H) wherein R¹ represents a methyl group, Q represents Q7, L² represents NH, R⁷ represents an ethyl group, and R¹⁷ represents any substituent selected from Group X (hereinafter, referred to as Compound group SX328).

A compound according to Compound (1H) wherein R¹ represents a chlorine atom, Q represents Q7, L² represents NH, R⁷ represents an ethyl group, and R¹⁷ represents any substituent selected from Group X (hereinafter, referred to as Compound group SX329).

A compound according to Compound (1H) wherein R¹ represents a methyl group, Q represents Q8A, R⁷ represents a methyl group, and R¹⁷ represents any substituent selected from Group X (hereinafter, referred to as Compound group SX330).

A compound according to Compound (1H) wherein R¹ represents a chlorine atom, Q represents Q8A, R⁷ represents a methyl group, and R¹⁷ represents any substituent selected from Group X (hereinafter, referred to as Compound group SX331).

A compound according to Compound (1H) wherein R¹ represents a methyl group, Q represents Q8A, R⁷ represents an ethyl group, and R¹⁷ represents any substituent selected from Group X (hereinafter, referred to as Compound group SX332).

A compound according to Compound (1H) wherein R¹ represents a chlorine atom, Q represents Q8A, R⁷ represents an ethyl group, and R¹⁷ represents any substituent selected from Group X (hereinafter, referred to as Compound group SX333).

A compound according to Compound (1H) wherein R¹ represents a methyl group, Q represents Q8D, R⁷ represents a methyl group, and R¹⁷ represents any substituent selected from Group X (hereinafter, referred to as Compound group SX334).

A compound according to Compound (1H) wherein R¹ represents a chlorine atom, Q represents Q8D, R⁷ represents a methyl group, and R¹⁷ represents any substituent selected from Group X (hereinafter, referred to as Compound group SX335).

A compound according to Compound (1H) wherein R¹ represents a methyl group, Q represents Q8D, R⁷ represents an ethyl group, and R¹⁷ represents any substituent selected from Group X (hereinafter, referred to as Compound group SX336).

A compound according to Compound (1H) wherein R¹ represents a chlorine atom, Q represents Q8D, R⁷ represents an ethyl group, and R¹⁷ represents any substituent selected from Group X (hereinafter, referred to as Compound group SX337).

A compound according to Compound (1H) wherein R¹ represents a methyl group, Q represents Q8J, R⁷ represents a methyl group, and R¹⁷ represents any substituent selected from Group X (hereinafter, referred to as Compound group SX338).

A compound according to Compound (1H) wherein R¹ represents a chlorine atom, Q represents Q8J, R⁷ represents a methyl group, and R¹⁷ represents any substituent selected from Group X (hereinafter, referred to as Compound group SX339).

A compound according to Compound (1H) wherein R¹ represents a methyl group, Q represents Q8J, R⁷ represents an ethyl group, and R¹⁷ represents any substituent selected from Group X (hereinafter, referred to as Compound group SX340).

A compound according to Compound (1H) wherein R¹ represents a chlorine atom, Q represents Q8J, R⁷ represents an ethyl group, and R¹⁷ represents any substituent selected from Group X (hereinafter, referred to as Compound group SX341).

A compound represented by formula (1I) (hereinafter, referred to as Compound (1I)):

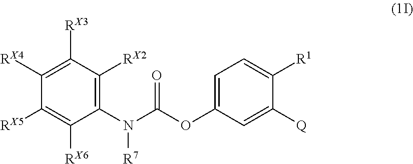

wherein R¹ represents a methyl group, Q represents Q3, R³ represents a methoxy group, R⁷ represents a methyl group, and a combination of R^{X2}, R^{X3}, R^{X4}, R^{X5} and R^{X6} represents any combination described in Combination A (hereinafter, referred to as Compound group SX342).

A compound according to Compound (1I) wherein R¹ represents a chlorine atom, Q represents Q3, R³ represents a methoxy group, R⁷ represents a methyl group, and a combination of R^{X2}, R^{X3}, R^{X4}, R^{X5} and R^{X6} represents any combination described in Combination A (hereinafter, referred to as Compound group SX343).

A compound according to Compound (1I) wherein R¹ represents a methyl group, Q represents Q3, R³ represents a methoxy group, R⁷ represents an ethyl group, and a combination of R^{X2}, R^{X3}, R^{X4}, R^{X5} and R^{X6} represents any combination described in Combination A (hereinafter, referred to as Compound group SX344).

A compound according to Compound (1I) wherein R¹ represents a chlorine atom, Q represents Q3, R³ represents a methoxy group, R⁷ represents an ethyl group, and a combination of R^{X2}, R^{X3}, R^{X4}, R^{X5} and R^{X6} represents any combination described in Combination A (hereinafter, referred to as Compound group SX345).

A compound according to Compound (1I) wherein R¹ represents a methyl group, Q represents Q3, R³ represents a methoxy group, R⁷ represents a cyclopropyl group, and a combination of R^{X2}, R^{X3}, R^{X4}, R^{X5} and R^{X6} represents any combination described in Combination A (hereinafter, referred to as Compound group SX346).

A compound according to Compound (1I) wherein R¹ represents a chlorine atom, Q represents Q3, R³ represents a methoxy group, R⁷ represents a cyclopropyl group, and a combination of R^{X2}, R^{X3}, R^{X4}, R^{X5} and R^{X6} represents any combination described in Combination A (hereinafter, referred to as Compound group SX347).

A compound according to Compound (1I) wherein R¹ represents a methyl group, Q represents Q4, R⁷ represents a methyl group, and a combination of R^{X2}, R^{X3}, R^{X4}, R^{X5} and R^{X6} (hereinafter, referred to as Compound group SX348).

A compound according to Compound (1I) wherein R¹ represents a chlorine atom, Q represents Q4, R⁷ represents a methyl group, and a combination of R^{X2}, R^{X3}, R^{X4}, R^{X5} and R^{X6} represents any combination described in Combination A (hereinafter, referred to as Compound group SX349).

A compound according to Compound (1I) wherein R¹ represents a methyl group, Q represents Q4, R⁷ represents an ethyl group, and a combination of R^{X2}, R^{X3}, R^{X4}, R^{X5} and R^{X6} represents any combination described in Combination A (hereinafter, referred to as Compound group SX350).

A compound according to Compound (1I) wherein R¹ represents a chlorine atom, Q represents Q4, R⁷ represents an ethyl group, and a combination of R^{X2}, R^{X3}, R^{X4}, R^{X5} and R^{X6} represents any combination described in Combination A (hereinafter, referred to as Compound group SX351).

A compound according to Compound (1I) wherein R¹ represents a methyl group, Q represents Q4, R⁷ represents a cyclopropyl group, and a combination of R^{X2}, R^{X3}, R^{X4}, R^{X5} and R^{X6} represents any combination described in Combination A (hereinafter, referred to as Compound group SX352).

A compound according to Compound (1I) wherein R¹ represents a chlorine atom, Q represents Q4, R⁷ represents a cyclopropyl group, and a combination of R^{X2}, R^{X3}, R^{X4}, R^{X5} and R^{X6} represents any combination described in Combination A (hereinafter, referred to as Compound group SX353).

A compound according to Compound (1I) wherein R¹ represents a methyl group, Q represents Q5, R⁷ represents a methyl group, and a combination of R^{X2}, R^{X3}, R^{X4}, R^{X5} and R^{X6} represents any combination described in Combination A (hereinafter, referred to as Compound group SX354).

A compound according to Compound (1I) wherein R¹ represents a chlorine atom, Q represents Q5, R⁷ represents a methyl group, and a combination of R^{X2}, R^{X3}, R^{X4}, R^{X5} and R^{X6} represents any combination described in Combination A (hereinafter, referred to as Compound group SX355).

A compound according to Compound (1I) wherein R¹ represents a methyl group, Q represents Q5, R⁷ represents an ethyl group, and a combination of R^{X2}, R^{X3}, R^{X4}, R^{X5} and R^{X6} represents any combination described in Combination A (hereinafter, referred to as Compound group SX356).

A compound according to Compound (1I) wherein R¹ represents a chlorine atom, Q represents Q5, R⁷ represents an ethyl group, and a combination of R^{X2}, R^{X3}, R^{X4}, R^{X5} and R^{X6} represents any combination described in Combination A (hereinafter, referred to as Compound group SX357).

A compound according to Compound (1I) wherein R¹ represents a methyl group, Q represents Q5, R⁷ represents a cyclopropyl group, and a combination of R^{X2}, R^{X3}, R^{X4}, R^{X5} and R^{X6} represents any combination described in Combination A (hereinafter, referred to as Compound group SX358).

A compound according to Compound (1I) wherein R¹ represents a chlorine atom, Q represents Q5, R⁷ represents a cyclopropyl group, and a combination of R^{X2}, R^{X3}, R^{X4}, R^{X5} and R^{X6} represents any combination described in Combination A (hereinafter, referred to as Compound group SX359).

A compound according to Compound (1I) wherein R¹ represents a methyl group, Q represents Q6, R⁷ represents a methyl group, and a combination of R^{X2}, R^{X3}, R^{X4}, R^{X5} and R^{X6} represents any combination described in Combination A (hereinafter, referred to as Compound group SX360).

A compound according to Compound (1I) wherein R¹ represents a chlorine atom, Q represents Q6, R⁷ represents a methyl group, and a combination of R^{X2}, R^{X3}, R^{X4}, R^{X5} and R^{X6} represents any combination described in Combination A (hereinafter, referred to as Compound group SX361).

A compound according to Compound (1I) wherein R¹ represents a methyl group, Q represents Q6, R⁷ represents an ethyl group, and a combination of R^{X2}, R^{X3}, R^{X4}, R^{X5} and R^{X6} represents any combination described in Combination A (hereinafter, referred to as Compound group SX362).

A compound according to Compound (1I) wherein R¹ represents a chlorine atom, Q represents Q6, R⁷ represents an ethyl group, and a combination of R^{X2}, R^{X3}, R^{X4}, R^{X5} and R^{X6} represents any combination described in Combination A (hereinafter, referred to as Compound group SX363).

A compound according to Compound (1I) wherein R¹ represents a methyl group, Q represents Q6, R⁷ represents a cyclopropyl group, and a combination of R^{X2}, R^{X3}, R^{X4}, R^{X5} and R^{X6} represents any combination described in Combination A (hereinafter, referred to as Compound group SX364).

A compound according to Compound (1I) wherein R¹ represents a chlorine atom, Q represents Q6, R⁷ represents a cyclopropyl group, and a combination of R^{X2}, R^{X3}, R^{X4}, R^{X5} and R^{X6} represents any combination described in Combination A (hereinafter, referred to as Compound group SX365).

A compound according to Compound (1I) wherein R¹ represents a methyl group, Q represents Q7, L² represents an oxygen atom, R⁷ represents a methyl group, and a combination of R^{X2}, R^{X3}, R^{X4}, R^{X5} and R^{X6} represents any combination described in Combination A (hereinafter, referred to as Compound group SX366).

A compound according to Compound (1I) wherein R¹ represents a chlorine atom, Q represents Q7, L² represents an oxygen atom, R⁷ represents a methyl group, and a combination of R^{X2}, R^{X3}, R^{X4}, R^{X5} and R^{X6} represents any combination described in Combination A (hereinafter, referred to as Compound group SX367).

A compound according to Compound (1I) wherein R¹ represents a methyl group, Q represents Q7, L² represents an oxygen atom, R⁷ represents an ethyl group, and a combination of R^{X2}, R^{X3}, R^{X4}, R^{X5} and R^{X6} represents any combination described in Combination A (hereinafter, referred to as Compound group SX368).

A compound according to Compound (1I) wherein R¹ represents a chlorine atom, Q represents Q7, L² represents an oxygen atom, R⁷ represents an ethyl group, and a combination of R^{X2}, R^{X3}, R^{X4}, R^{X5} and R^{X6} represents any combination described in Combination A (hereinafter, referred to as Compound group SX369).

A compound according to Compound (1I) wherein R¹ represents a methyl group, Q represents Q7, L² represents NH, R⁷ represents a methyl group, and a combination of R^{X2}, R^{X3}, R^{X4}, R^{X5} and R^{X6} represents any combination described in Combination A (hereinafter, referred to as Compound group SX370).

A compound according to Compound (1I) wherein R¹ represents a chlorine atom, Q represents Q7, L² represents NH, R⁷ represents a methyl group, and a combination of R^{X2}, R^{X3}, R^{X4}, R^{X5} and R^{X6} represents any combination described in Combination A (hereinafter, referred to as Compound group SX371).

A compound according to Compound (1I) wherein R¹ represents a methyl group, Q represents Q7, L² represents NH, R⁷ represents an ethyl group, and a combination of R^{X2}, R^{X3}, R^{X4}, R^{X5} and R^{X6} represents any combination described in Combination A (hereinafter, referred to as Compound group SX372).

A compound according to Compound (1I) wherein R¹ represents a chlorine atom, Q represents Q7, L² represents NH, R⁷ represents an ethyl group, and a combination of R^{X2}, R^{X3}, R^{X4}, R^{X5} and R^{X6} represents any combination described in Combination A (hereinafter, referred to as Compound group SX373).

A compound according to Compound (1I) wherein R¹ represents a methyl group, Q represents Q8A, R⁷ represents a methyl group, and a combination of R^{X2}, R^{X3}, R^{X4}, R^{X5} and R^{X6} represents any combination described in Combination A (hereinafter, referred to as Compound group SX374).

A compound according to Compound (1I) wherein R¹ represents a chlorine atom, Q represents Q8A, R⁷ represents a methyl group, and a combination of R^{X2}, R^{X3}, R^{X4}, R^{X5} and R^{X6} represents any combination described in Combination A (hereinafter, referred to as Compound group SX375).

A compound according to Compound (1I) wherein R¹ represents a methyl group, Q represents Q8A, R⁷ represents an ethyl group, and a combination of R^{X2}, R^{X3}, R^{X4}, R^{X5} and R^{X6} represents any combination described in Combination A (hereinafter, referred to as Compound group SX376).

A compound according to Compound (1I) wherein R¹ represents a chlorine atom, Q represents Q8A, R⁷ represents an ethyl group, and a combination of R^{X2}, R^{X3}, R^{X4}, R^{X5} and R^{X6} represents any combination described in Combination A (hereinafter, referred to as Compound group SX377).

A compound according to Compound (1I) wherein R¹ represents a methyl group, Q represents Q8D, R⁷ represents a methyl group, and a combination of R^{X2}, R^{X3}, R^{X4}, R^{X5} and R^{X6} represents any combination described in Combination A (hereinafter, referred to as Compound group SX378).

A compound according to Compound (1I) wherein R¹ represents a chlorine atom, Q represents Q8D, R⁷ represents a methyl group, and a combination of R^{X2}, R^{X3}, R^{X4}, R^{X5} and R^{X6} represents any combination described in Combination A (hereinafter, referred to as Compound group SX379).

A compound according to Compound (1I) wherein R¹ represents a methyl group, Q represents Q8D, R⁷ represents an ethyl group, and a combination of R^{X2}, R^{X3}, R^{X4}, R^{X5} and R^{X6} represents any combination described in Combination A (hereinafter, referred to as Compound group SX380).

A compound according to Compound (1I) wherein R¹ represents a chlorine atom, Q represents Q8D, R⁷ represents an ethyl group, and a combination of R^{X2}, R^{X3}, R^{X4}, R^{X5} and R^{X6} represents any combination described in Combination A (hereinafter, referred to as Compound group SX381).

A compound according to Compound (1I) wherein R¹ represents a methyl group, Q represents Q8J, R⁷ represents a methyl group, and a combination of R^{X2}, R^{X3}, R^{X4}, R^{X5} and R^{X6} represents any combination described in Combination A (hereinafter, referred to as Compound group SX382).

A compound according to Compound (1I) wherein R¹ represents a chlorine atom, Q represents Q8J, R⁷ represents a methyl group, and a combination of R^{X2}, R^{X3}, R^{X4}, R^{X5} and R^{X6} represents any combination described in Combination A (hereinafter, referred to as Compound group SX383).

A compound according to Compound (1I) wherein R¹ represents a methyl group, Q represents Q8J, R⁷ represents an ethyl group, and a combination of R^{X2}, R^{X3}, R^{X4}, R^{X5} and R^{X6} represents any combination described in Combination A (hereinafter, referred to as Compound group SX384).

A compound according to Compound (1I) wherein R¹ represents a chlorine atom, Q represents Q8J, R⁷ represents an ethyl group, and a combination of R^{X2}, R^{X3}, R^{X4}, R^{X5} and R^{X6} represents any combination described in Combination A (hereinafter, referred to as Compound group SX385).

A compound represented by formula (1J) (hereinafter, referred to as Compound (1J)):

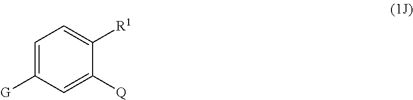

[wherein G represents any one of formulae G1 to G35

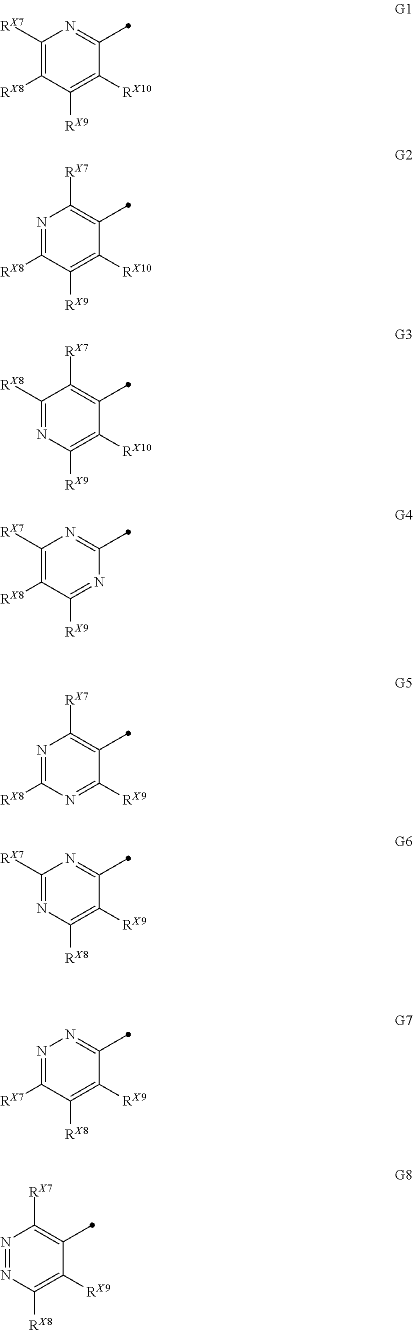

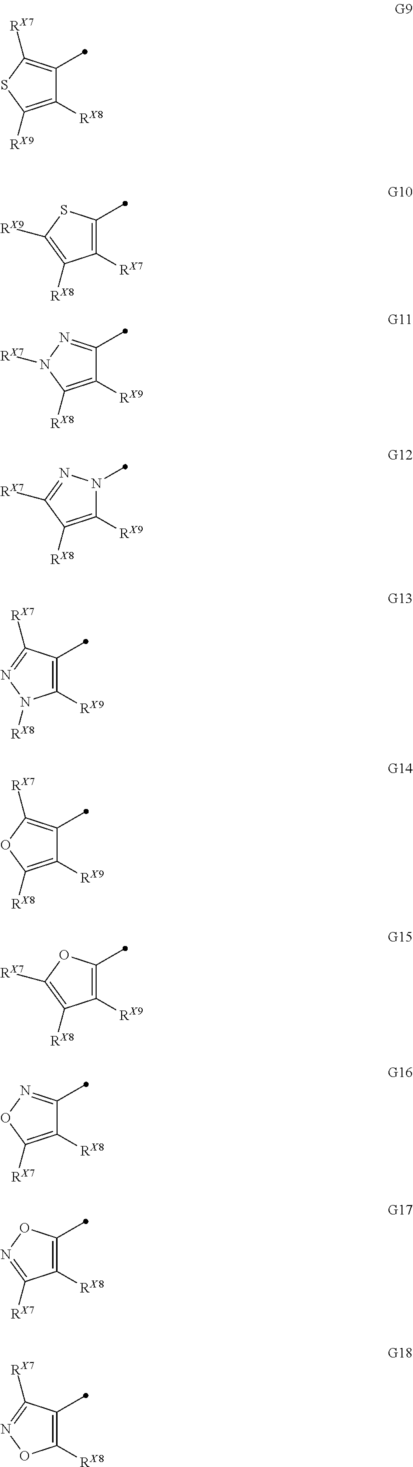

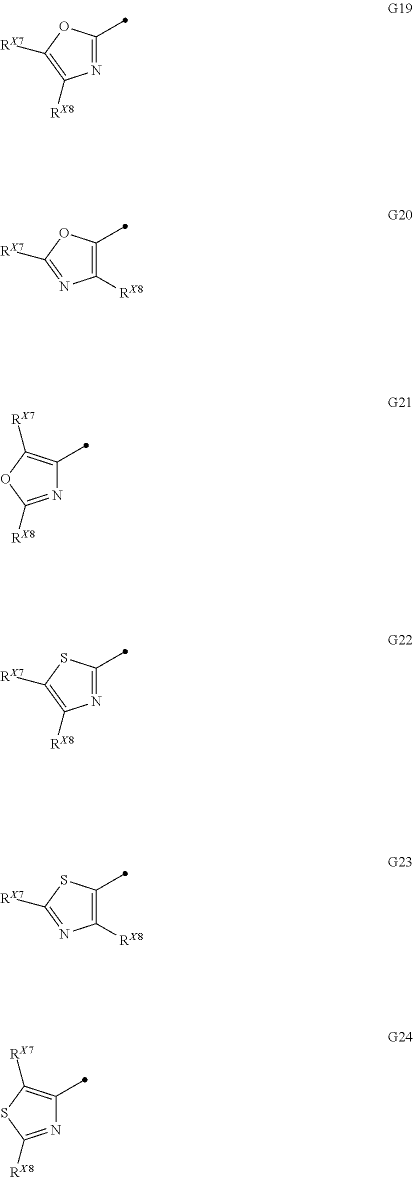

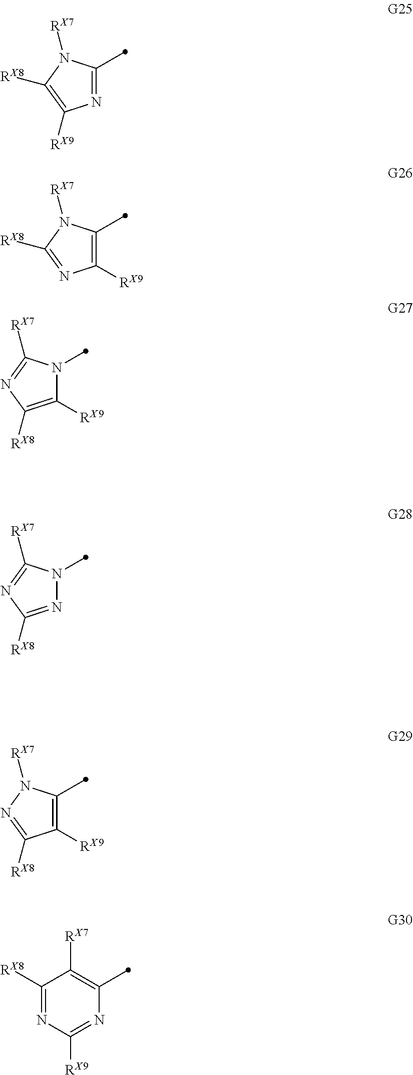

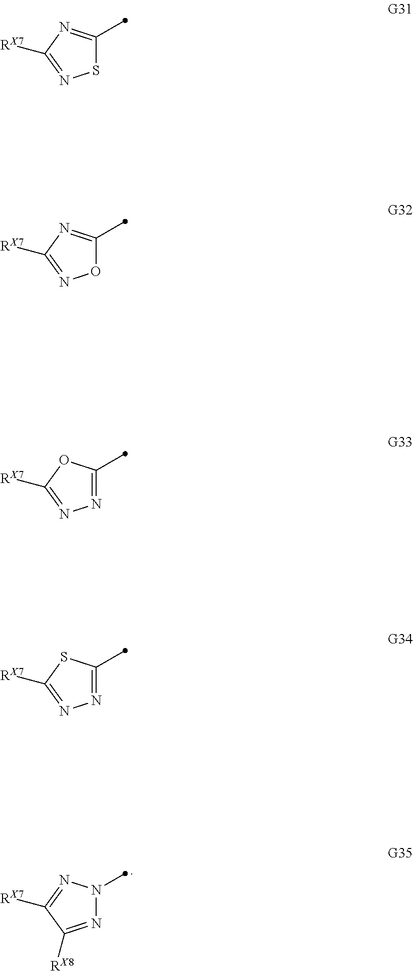

wherein R¹ represents a methyl group, Q represents Q3, R³ represents a methoxy group, and a combination of structure of G and substituents of R^{X7}, R^{X8}, R^{X9} and R^{X10} applied to the structure of G represents any combination described in Combination B (hereinafter, referred to as Compound group SX386).

Combination B consists of substituent numbers ZB1 to ZB1562. Substituent numbers ZB1 to ZB1562 represent combinations of structure of G and substituents of R^{X7}, R^{X8}, R^{X9} and R^{X10} applied to the structure of G according to Compound (1J), compounds represented by formula (2B), and compounds represented by formula (2C); combinations of structure of G³ and substituents of R^{X7}, R^{X8}, R^{X9} and R^{X10} applied to the structure of G³ according to compounds represented by formula (2K), compounds represented by formula (2M), and compounds represented by formula (20); and combinations of structure of G⁵ and substituents of R^{X7}, R^{X8}, R^{X9} and R^{X10} applied to the structure of G⁵ according to compounds represented by formula (3C), compounds represented by formula (3E), and compounds represented by formula (2T). And hereinafter, they are referred to as [substituent number; G, R^{X7}, R^{X8}, R^{X9}, R^{X1}]. For example, the substituent number ZB2 refers to a combination wherein G, G³ or G⁵ represents G1, R^{X7} represents a methyl group, and each of R^{X8}, R^{X9} and R^{X10} represents a hydrogen atom.
Combination B: [ZB1;G1,H,H,H,H], [ZB2;G1,Me,H,H,H], [ZB3;G1,Et,H,H,H], [ZB4;G 1,Pr,H,H,H], [ZB5;G1,i-Pr,H,H,H], [ZB6;G1,c-Pr,H,H,H], [ZB7;G1,Ph,H,H,H], [ZB8;G1,OMe,H,H,H], [ZB9;G1,OEt, H,H,H], [ZB10;G1,OPh,H,H,H], [ZB11;G1,CF₃,H,H,H], [ZB12;G1,F,H,H,H], [ZB13;G1,Cl,H,H,H], [ZB14;G1,Br,H,H,H], [ZB15;G,CN,H,H,H], [ZB16;G1,H,Me,H,H], [ZB17;G1,H,Et,H,H], [ZB18;G1,H,Pr,H,H], [ZB19;G1,H,i-Pr,H,H], [ZB20;G1,H,c-Pr,H,H], [ZB21;G1,H,Ph,H,H], [ZB22;G1,H,OMe,H,H], [ZB23;G1,H,O Et,H,H], [ZB24;G1,H,OPh,H,H], [ZB25;G1,H,CF₃,H,H], [ZB26;G1,H, F,H,H], [ZB27;G1,H,Cl,H,H], [ZB28;G1,H,Br,H,H], [ZB29;G1,H,CN, H,H], [ZB30;G1,H,H,Me,H], [ZB31;G1,H,H,Et,H], [ZB32;G1,H,H,Pr, H], [ZB33;G1,H,H,i-Pr,H], [ZB34;G1,H,H,c-Pr,H], [ZB35;G1,H,H,Ph,H], [ZB36;G1,H,H,OMe,H], [ZB37;G1,H,H,O Et,H], [ZB38;G1,H,H,OPh,H], [ZB39;G1,H,H,CF₃,H], [ZB40;G1,H,H, F,H], [ZB41;G1,H,H,Cl,H], [ZB42;G1,H,H,Br,H], [ZB43;G1,H,H,CN, H], [ZB44;G1,H,H,H,Me], [ZB45;G1,H,H,H,Et], [ZB46;G1,H,H,H,Pr], [ZB47;G1,H,H,H,i-Pr], [ZB48;G1,H,H,H,c-Pr], [ZB49;G1,H,H,H,Ph], [ZB50;G1,H,H,H,OMe], [ZB51;G1,H,H,H,O Et], [ZB52;G1,H,H,H,OPh], [ZB53;G1,H,H,H,CF₃], [ZB54;G1,H,H,H, F], [ZB55;G1,H,H,H,Cl], [ZB56;G1,H,H,H,Br], [ZB57;G1,H,H,H,CN], [ZB58;G2,H,H,H,H], [ZB59;G2,Me,H,H,H], [ZB60;G2,Et,H,H,H], [Z B61;G2,Pr,H,H,H], [ZB62;G2,i-Pr,H,H,H], [ZB63;G2,c-Pr,H,H,H], [ZB64;G2,Ph,H,H,H], [ZB65;G2,OMe,H,H,H], [ZB66;G2,O Et,H,H,H], [ZB67;G2,OPh,H,H,H], [ZB68;G2,CF₃,H,H,H], [ZB69;G2, F,H,H,H], [ZB70;G2,Cl,H,H,H], [ZB71;G2,Br,H,H,H], [ZB72;G2,CN, H,H,H], [ZB73;G2,H,Me,H,H], [ZB74;G2,H,Et,H,H], [ZB75;G2,H,Pr, H,H], [ZB76;G2,H,i-Pr,H,H], [ZB77;G2,H,c-Pr,H,H], [ZB78;G2,H,Ph,H,H], [ZB79;G2,H,OMe,H,H], [ZB80;G2,H,O Et,H,H], [ZB81;G2,H,OPh,H,H], [ZB82;G2,H,CF₃,H,H], [ZB83;G2,H, F,H,H], [ZB84;G2,H,Cl,H,H], [ZB85;G2,H,Br,H,H], [ZB86;G2,H,CN, H,H], [ZB87;G2,H,H,Me,H], [ZB88;G2,H,H,Et,H], [ZB89;G2,H,H,Pr, H], [ZB90;G2,H,H,i-Pr,H], [ZB91;G2,H,H,c-Pr,H], [ZB92;G2,H,H,Ph,H], [ZB93;G2,H,H,OMe,H], [ZB94;G2,H,H,O Et,H], [ZB95;G2,H,H,OPh,H], [ZB96;G2,H,H,CF₃,H], [ZB97;G2,H,H, F,H], [ZB98;G2,H,H,Cl,H], [ZB99;G2,H,H,Br,H], [ZB100;G2,H,H,CN,H], [ZB101;G2,H,H,H,Me], [ZB102;G2,H,H,H,Et], [ZB103;G2,H,H,H,Pr], [ZB104;G2,H,H,H,i-Pr], [ZB105;G2,H,H,H,c-Pr], [ZB106;G2,H,H,H,Ph], [ZB107;G2,H,H,H,OMe], [ZB108;G2,H,H, H,OEt], [ZB109;G2,H,H,H,OPh], [ZB110;G2,H,H,H,CF₃], [ZB111;G2, H,H,H,F], [ZB112;G2,H,H,H,Cl], [ZB113;G2,H,H,H,Br], [ZB114;G2, H,H,H,CN], [ZB115;G3,H,H,H,H], [ZB116;G3,Me,H,H,H], [ZB117;G3, Et,H,H,H], [ZB118;G3,Pr,H,H,H], [ZB119;G3,i-Pr,H,H,H], [ZB120;G3,c-Pr,H,H,H], [ZB121;G3,Ph,H,H,H], [ZB122;G3,OMe,H,H,H], [ZB123;G 3,OEt,H,H,H], [ZB124;G3,OPh,H,H,H], [ZB125;G3,CF₃,H,H,H], [ZB1 26;G3,F,H,H,H], [ZB127;G3,Cl,H,H,H], [ZB128;G3,Br,H,H,H], [ZB1 29;G3,CN,H,H,H], [ZB130;G3,H,Me,H,H], [ZB131;G3,H,Et,H,H], [ZB 132;G3,H,Pr,H,H], [ZB133;G3,H,i-Pr,H,H], [ZB134;G3,H,c-Pr,H,H], [ZB135;G3,H,Ph,H,H], [ZB136;G3,H,OMe,H,H], [ZB137;G3, H,OEt,H,H], [ZB138;G3,H,OPh,H,H], [ZB139;G3,H,CF₃,H,H], [ZB140;G3,H,F,H,H], [ZB141;G3,H,Cl,H,H], [ZB142;G3,H,Br,H,H], [ZB143;G3,H,CN,H,H], [ZB144;G3,H,H,Me,H], [ZB145;G3,H,H,Et,H], [ZB14 6;G3,H,H,Pr,H], [ZB147;G3,H,H,i-Pr,H], [ZB148;G3,H,H,c-Pr,H], [ZB149;G3,H,H,Ph,H], [ZB150;G3,H,H,OMe,H], [ZB151;G3,H, H,OEt,H], [ZB152;G3,H,H,OPh,H], [ZB153;G3,H,H,CF₃,H], [ZB154;G 3,H,H,F,H], [ZB155;G3,H,H,Cl,H], [ZB156;G3,H,H,Br,H], [ZB157;G 3,H,H,CN,H], [ZB158;G3,H,H,H,Me], [ZB159;G3,H,H,H,Et], [ZB160; G3,H,H,H,Pr], [ZB161;G3,H,H,H,i-Pr], [ZB162;G3,H,H,H,c-Pr], [ZB163;G3,H,H,H,Ph], [ZB164;G3,H,H,H,OMe], [ZB165;G3,H,H, H,OEt], [ZB166;G3,H,H,H,OPh], [ZB167;G3,H,H,H,CF₃], [ZB168;G3, H,H,H,F], [ZB169;G3,H,H,H,Cl], [ZB170;G3,H,H,H,Br], [ZB171;G3, H,H,H,CN], [ZB172;G4,H,H,H,-], [ZB173;G4,Me,H,H,-], [ZB174;G4, Et,H,H,-], [ZB175;G4,Pr,H,H,-], [ZB176;G4,i-Pr,H,H,-], [ZB177;G4,c-Pr,H,H,-], [ZB178;G4,Ph,H,H,-], [ZB179;G4,OMe,H,H,-], [ZB180;G 4,OEt,H,H,-], [ZB181;G4,OPh,H,H,-], [ZB182;G4,CF₃,H,H,-], [ZB1 83;G4,F,H,H,-], [ZB184;G4,Cl,H,H,-], [ZB185;G4,Br,H,H,-], [ZB1 86;G4,CN,H,H,-], [ZB187;G4,H,Me,H,-], [ZB188;G4,H,Et,H,-], [ZB 189;G4,H,Pr,H,-], [ZB190;G4,H,i-Pr,H,-], [ZB191;G4,H,c-Pr,H,-], [ZB192;G4,H,Ph,H,-], [ZB193;G4,H,OMe,H,-], [ZB194;G4, H,OEt,H,-], [ZB195;G4,H,OPh,H,-], [ZB196;G4,H,CF₃,H,-], [ZB197;G4,H,F,H,-], [ZB198;G4,H,Cl,H,-], [ZB199;G4,H,Br,H,-], [ZB200;G4,H,CN,H,-], [ZB201;G4,H,H,Me,-], [ZB202;G4,H,H,Et,-], [ZB203;G4,H,H,Pr,-], [ZB204;G4,H,H,i-Pr,-], [ZB205;G4,H,H,c-Pr,-], [ZB206;G4,H,H,Ph,-], [ZB207;G4,H,H,OMe,-], [ZB208;G4,H, H,OEt,-], [ZB209;G4,H,H,OPh,-], [ZB210;G4,H,H,CF₃,-], [ZB211;G 4,H,H,F,-], [ZB212;G4,H,H,Cl,-], [ZB213;G4,H,H,Br,-], [ZB214;G 4,H,H,CN, -], [ZB215;G5,H,H,H, -], [ZB216;G5,Me,H,H, -], [ZB217;G 5,Et,H,H,-], [ZB218;G5,Pr,H,H,-], [ZB219;G5,i-Pr,H,H,-], [ZB220;G5,c-Pr,H,H,-], [ZB221;G5,Ph,H,H,-], [ZB222;G5,OMe,H,H,-], [ZB223;G 5,OEt,H,H,-], [ZB224;G5,OPh,H,H,-], [ZB225;G5,CF₃,H,H,-], [ZB2 26;G5,F,H,H,-], [ZB227;G5,Cl,H,H,-], [ZB228;G5,Br,H,H,-], [ZB2 29;G5,CN,H,H,H-], [ZB230;G5,H,Me,H,-], [ZB231;G5,H,Et,H,-], [ZB 232;G5,H,Pr,H,-], [ZB233;G5,H,i-Pr,H,-], [ZB234;G5,H,c-Pr,H,-], [ZB235;G5,H,Ph,H,-], [ZB236;G5,H,OMe,H,-], [ZB237;G5, H,OEt,H,-], [ZB238;G5,H,OPh,H,-], [ZB239;G5,H,CF₃,H,-], [ZB240;G5,H,F,H,-], [ZB241;G5,H,Cl,H,-], [ZB242;G5,H,Br,H,-], [ZB243;G5,H,CN,H,-], [ZB244;G5,H,H,Me,-], [ZB245;G5,H,H,Et,-], [ZB24 6;G5,H,H,Pr,-], [ZB247;G5,H,H,i-Pr,-], [ZB248;G5,H,H,c-Pr,-], [ZB249;G5,H,H,Ph,-], [ZB250;G5,H,H,OMe,-], [ZB251;G5,H, H,OEt,-], [ZB252;G5,H,H,OPh,-], [ZB253;G5,H,H,CF₃,-], [ZB254;G 5,H,H,F,-], [ZB255;G5,H,H,Cl,-], [ZB256;G5,H,H,Br,-], [ZB257;G 5,H,H,CN,-], [ZB258;G6,H,H,H,-], [ZB259;G6,Me,H,H,-], [ZB260;G 6,Et,H,H,-], [ZB261;G6,Pr,H,H,-], [ZB262;G6,i-Pr,H,H,-], [ZB263;G6,c-Pr,H,H,-], [ZB264;G6,Ph,H,H,-], [ZB265;G6,OMe,H,H,-], [ZB266;G 6,OEt,H,H,-], [ZB267;G6,OPh,H,H,-], [ZB268;G6,CF₃,H,H,-], [ZB2 69;G6,F,H,H,-], [ZB270;G6,Cl,H,H,-], [ZB271;G6,Br,H,H,-], [ZB2 72;G6,CN,H,H,-], [ZB273;G6,H,Me,H,-], [ZB274;G6,H,Et,H,-], [ZB 275;G6,H,Pr,H,-], [ZB276;G6,H,i-Pr,H,-], [ZB277;G6,H,c-Pr,H,-], [ZB278;G6,H,Ph,H,-], [ZB279;G6,H,OMe,H,-], [ZB280;G6, H,OEt,H,-], [ZB281;G6,H,OPh,H,-], [ZB282;G6,H,CF₃,H,-], [ZB283;G6,H,F,H,-], [ZB284;G6,H,Cl,H,-], [ZB285;G6,H,Br,H,-], [ZB286;G6,H,CN,H,-], [ZB287;G6,H,H,Me,-], [ZB288;G6,H,H,Et,-], [ZB28 9;G6,H,H,Pr,-], [ZB290;G6,H,H,i-Pr,-], [ZB291;G6,H,H,c-Pr,-], [ZB292;G6,H,H,Ph,-], [ZB293;G6,H,H,OMe,-], [ZB294;G6,H, H,OEt,-], [ZB295;G6,H,H,OPh,-], [ZB296;G6,H,H,CF₃,-], [ZB297;G 6,H,H,F,-], [ZB298;G6,H,H,Cl,-], [ZB299;G6,H,H,Br,-], [ZB300;G 6,H,H,CN,-], [ZB301;G7,H,H,H,-], [ZB302;G7,Me,H,H,-], [ZB303;G 7,Et,H,H,-], [ZB304;G7,Pr,H,H,-], [ZB305;G7,i-Pr,H,H,-], [ZB306;G7,c-Pr,H,H,-], [ZB307;G7,Ph,H,H,-], [ZB308;G7,OMe,H,H,-], [ZB309;G 7,OEt,H,H,-], [ZB310;G7,OPh,H,H,-], [ZB311;G7,CF₃,H,H,-], [ZB3 12;G7,F,H,H,-], [ZB313;G7,Cl,H,H,-], [ZB314;G7,Br,H,H,-], [ZB3 15;G7,CN,H,H,-], [ZB316;G7,H,Me,H,-], [ZB317;G7,H,Et,H,-], [ZB 318;G7,H,Pr,H,-], [ZB319;G7,H,i-Pr,H,-], [ZB320;G7,H,c-Pr,H,-], [ZB321;G7,H,Ph,H,-], [ZB322;G7,H,OMe,H,-], [ZB323;G7, H,OEt,H,-], [ZB324;G7,H,OPh,H,-], [ZB325;G7,H,CF₃,H,-], [ZB326;G7,H,F,H,-], [ZB327;G7,H,Cl,H,-], [ZB328;G7,H,Br,H,-], [ZB329;G7,H,CN,H,-], [ZB330;G7,H,H,Me,-], [ZB331;G7,H,H,Et,-], [ZB33 2;G7,H,H,Pr,-], [ZB333;G7,H,H,i-Pr,-], [ZB334;G7,H,H,c-Pr,-], [ZB335;G7,H,H,Ph,-], [ZB336;G7,H,H,OMe,-], [ZB337;G7,H, H,OEt,-], [ZB338;G7,H,H,OPh,-], [ZB339;G7,H,H,CF₃,-], [ZB340;G 7,H,H,F,-], [ZB341;G7,H,H,Cl,-], [ZB342;G7,H,H,Br,-], [ZB343;G 7,H,H,CN,-], [ZB344;G8,H,H,H,-], [ZB345;G8,Me,H,H,-], [ZB346;G 8,Et,H,H,-], [ZB347;G8,Pr,H,H,-], [ZB348;G8,i-Pr,H,H,-], [ZB349;G8,c-Pr,H,H,-], [ZB350;G8,Ph,H,H,-], [ZB351;G8,OMe,H,H,-], [ZB352;G 8,OEt,H,H,-], [ZB353;G8,OPh,H,H,-], [ZB354;G8,CF₃,H,H,-], [ZB3 55;G8,F,H,H,-], [ZB356;G8,Cl,H,H,-], [ZB357;G8,Br,H,H,-], [ZB3 58;G8,CN,H,H,-], [ZB359;G8,H,Me,H,-], [ZB360;G8,H,Et,H,-], [ZB 361;G8,H,Pr,H,-], [ZB362;G8,H,i-Pr,H,-], [ZB363;G8,H,c-Pr,H,-], [ZB364;G8,H,Ph,H,-], [ZB365;G8,H,OMe,H,-], [ZB366;G8, H,OEt,H,-], [ZB367;G8,H,OPh,H,-], [ZB368;G8,H,CF₃,H,-], [ZB369;G8,H,F,H,-], [ZB370;G8,H,Cl,H,-], [ZB371;G8,H,Br,H,-], [ZB372;G8,H,CN,H,-], [ZB373;G8,H,H,Me,-], [ZB374;G8,H,H,Et,-], [ZB37 5;G8,H,H,Pr,-], [ZB376;G8,H,H,i-Pr,-], [ZB377;G8,H,H,c-Pr,-], [ZB378;G8,H,H,Ph,-], [ZB379;G8,H,H,OMe,-], [ZB380;G8,H, H,OEt,-], [ZB381;G8,H,H,OPh,-], [ZB382;G8,H,H,CF₃,-], [ZB383;G 8,H,H,F,-], [ZB384;G8,H,H,Cl,-], [ZB385;G8,H,H,Br,-], [ZB386;G 8,H,H,CN,-], [ZB387;G9,H,H,H,-], [ZB388;G9,Me,H,H,-], [ZB389;G 9,Et,H,H,-], [ZB390;G9,Pr,H,H,-], [ZB391;G9,i-Pr,H,H,-], [ZB392;G9,c-Pr,H,H,-], [ZB393;G9,Ph,H,H,-], [ZB394;G9,OMe,H,H,-], [ZB395;G 9,OEt,H,H,-], [ZB396;G9,OPh,H,H,-], [ZB397;G9,CF₃,H,H,-], [ZB3 98;G9,F,H,H,-], [ZB399;G9,Cl,H,H,-], [ZB400;G9,Br,H,H,-], [ZB401;G9,CN,H,H,-], [ZB402;G9,H,Me,H,-], [ZB403;G9,H,Et,H,-], [ZB404;G9,H,Pr,H,-], [ZB405;G9,H,i-Pr,H,-], [ZB406;G9,H,c-Pr,H,-], [ZB407;G9,H,Ph,H,-], [ZB408;G9,H,OMe,H,-], [ZB409;G9, H,OEt,H,-], [ZB410;G9,H,OPh,H,-], [ZB411;G9,H,CF₃,H,-], [ZB412;G9,H,F,H,-], [ZB413;G9,H,Cl,H,-], [ZB414;G9,H,Br,H,-], [ZB415;G9,H,CN,H,-], [ZB416;G9,H,H,Me,-], [ZB417;G9,H,H,Et,-], [ZB41 8;G9,H,H,Pr,-], [ZB419;G9,H,H,i-Pr,-], [ZB420;G9,H,H,c-Pr,-], [ZB421;G9,H,H,Ph,-], [ZB422;G9,H,H,OMe,-], [ZB423;G9,H, H,OEt,-], [ZB424;G9,H,H,OPh,-], [ZB425;G9,H,H,CF₃,-], [ZB426;G 9,H,H,F,-], [ZB427;G9,H,H,Cl,-], [ZB428;G9,H,H,Br,-], [ZB429;G 9,H,H,CN,-], [ZB430;G10,H,H,H,-], [ZB431;G10,Me,H,H,-], [ZB432;G10,Et,H,H,-], [ZB433;G10,Pr,H,H,-], [ZB434;G10,i-Pr,H,H,-], [ZB435;G10,c-Pr,H,H,-], [ZB436;G10,Ph,H,H,-], [ZB437;G10,OMe,H,H,-], [ZB438;G10,OEt,H,H,-], [ZB439;G10,OPh,H,H,-], [ZB440;G10,CF₃,H,H,-], [ZB441;G10,F,H,H,-], [ZB442;G10,Cl,H,H,-], [ZB443;G10,Br,H,H,-], [ZB444;G10,CN,H,H,-], [ZB445;G10,H,Me,H,-], [ZB446;G10,H, Et,H,-], [ZB447;G10,H,Pr,H,-], [ZB448;G10,H,i-Pr,H,-], [ZB449;G10,H,c-Pr,H,-], [ZB450;G10,H,Ph,H,-], [ZB451;G10,H,OMe,H,-], [ZB452;G 10,H,OEt,H, -], [ZB453;G10,H,OPh,H, -], [ZB454;G10,H,CF₃,H,-], [ZB455;G10,H,F,H,-], [ZB456;G10,H,Cl,H,-], [ZB457;G10,H,Br,H,-], [ZB458;G10,H,CN,H,-], [ZB459;G10,H,H,Me,-], [ZB460;G10,H,H, Et,-], [ZB461;G10,H,H,Pr,-], [ZB462;G10,H,H,i-Pr,-], [ZB463;G10,H,H,c-Pr,-], [ZB464;G10,H,H,Ph,-], [ZB465;G10,H,H,OMe,-], [ZB466;G10,H,H,OEt,-], [ZB467;G10,H,H,OPh,-], [ZB468;G10,H,H,CF₃,-], [ZB 469;G10,H,H,F,-], [ZB470;G10,H,H,Cl,-], [ZB471;G10,H,H,Br,-], [ZB472;G10,H,H,CN,-], [ZB473;G11,H,H,H,-], [ZB474;G11,H,Me,H,-], [ZB475;G11,H,Et,H,-], [ZB476;G11,H,Pr,H,-], [ZB477;G11,H,i-Pr,H,-], [ZB478;G11,H,c-Pr,H,-], [ZB479;G11,H,Ph,H,-], [ZB480;G11,H,OMe,H,-], [ZB481;G 11,H,OEt,H, -], [ZB482;G11,H,OPh,H, -], [ZB483;G11,H,CF₃,H,-], [ZB484;G11,HF,H,-], [ZB485;G11,H,Cl,H,-], [ZB486;G11,H,Br,H,-], [ZB487;G11,H,CN,H,-], [ZB488;G11,H,H,Me,-], [ZB489;G11,H,H,E t,-], [ZB490;G11,H,H,Pr,-], [ZB491;G11,H,H,i-Pr,-], [ZB492;G11,H,H,c-Pr,-], [ZB493;G11,H,H,Ph,-], [ZB494;G11,H,H,OMe,-], [ZB495;G11,H,H,OEt,-], [ZB496;G11,H,H,OPh,-], [ZB497;G11,H,H,CF₃,-], [ZB 498;G11,H,H,F,-], [ZB499;G11,H,H,Cl,-], [ZB500;G11,H,H,Br,-], [ZB501;G11,H,H,CN,-], [ZB502;G11,Me,H,H,-], [ZB503;G11,Me,Me, H,-], [ZB504;G11,Me,Et,H,-], [ZB505;G11,Me,Pr,H,-], [ZB506;G11,Me,i-Pr,H,-], [ZB507;G11,Me,c-Pr,H,-], [ZB508;G11,Me,Ph,H,-], [ZB509;G11,Me,OMe,H,-], [ZB510;G11,Me,OEt,H,-], [ZB511;G11,Me,OPh,H,-], [ZB512;G11,Me,CF₃,H,-], [ZB513;G11,Me,F,H,-], [ZB514;G11,Me,Cl,H,-], [ZB515;G11,M e,Br,H,-], [ZB516;G11,Me,CN,H,-], [ZB517;G11,Me,H,Me,-], [ZB51 8;G11,Me,H,Et,-], [ZB519;G11,Me,H,Pr,-], [ZB520;G11,Me,H,i-Pr,-], [ZB521;G11,Me,H,c-Pr,-], [ZB522;G11,Me,H,Ph,-], [ZB523;G11,Me,H,OMe,-], [ZB524;G 11,Me,H,OEt,-], [ZB525;G11,Me,H,OPh,-], [ZB526;G11,Me,H,CF₃,-], [ZB527;G11,Me,H,F,-], [ZB528;G11,Me,H,Cl,-], [ZB529;G11,Me, H,Br,-], [ZB530;G11,Me,H,CN,-], [ZB531;G12,H,H,H,-], [ZB532;G1 2,Me,H,H,-], [ZB533;G12,Et,H,H,-], [ZB534;G12,Pr,H,H,-], [ZB53 5;G12,i-Pr,H,H,-], [ZB536;G12,c-Pr,H,H,-], [ZB537;G12,Ph,H,H,-], [ZB538;G12,OMe,H,H,-], [ZB539;G12,OEt,H,H,-], [ZB540;G12,OPh,H,H,-], [ZB541;G12,CF₃,H,H,-], [ZB542;G12,F,H,H,-], [ZB543;G12,Cl,H,H,-], [ZB544;G12,Br,H,H,-], [ZB545;G12,CN,H,H,-], [ZB546;G12,H,Me,H,-], [ZB547;G12,H, Et,H,-], [ZB548;G12,H,Pr,H,-], [ZB549;G12,H,i-Pr,H,-], [ZB550;G12,H,c-Pr,H,-], [ZB551;G12,H,Ph,H,-], [ZB552;G12,H,OMe,H,-], [ZB553;G 12,H,OEt,H, -], [ZB554;G12,H,OPh,H, -], [ZB555;G12,H,CF₃,H,-], [ZB556;G12,H,F,H,-], [ZB557;G12,H,Cl,H,-], [ZB558;G12,H,Br,H,-], [ZB559;G12,H,CN,H,-], [ZB560;G12,H,H,Me,-], [ZB561;G12,H,H, Et,-], [ZB562;G12,H,H,Pr,-], [ZB563;G12,H,H,i-Pr,-], [ZB564;G12,H,H,c-Pr,-], [ZB565;G12,H,H,Ph,-], [ZB566;G12,H,H,OMe,-], [ZB567;G12,H,H,OEt,-], [ZB568;G12,H,H,OPh,-], [ZB569;G12,H,H,CF₃,-], [ZB 570;G12,H,H,F,-], [ZB571;G12,H,H,Cl,-], [ZB572;G12,H,H,Br,-], [ZB573;G12,H,H,CN,-], [ZB574;G13,H,H,H,-], [ZB575;G13,Me,H,H,-], [ZB576;G13,Et,H,H,-], [ZB577;G13,Pr,H,H,-], [ZB578;G13,i-Pr,H,H,-], [ZB579;G13,c-Pr,H,H,-], [ZB580;G13,Ph,H,H,-], [ZB581;G13,OMe,H,H,-], [ZB582;G13,OEt,H,H,-], [ZB583;G13,OPh,H,H,-], [ZB584;G13,CF₃,H,H,-],[ZB585;G13,F,H,H,-], [ZB586;G13,Cl,H,H,-], [ZB587;G13,Br,H,H, -], [ZB588;G13,CN,H,H, -], [ZB589;G13,H,H,Me, -], [ZB590;G13,H, H,Et,-], [ZB591;G13,H,H,Pr,-], [ZB592;G13,H,H,i-Pr,-], [ZB593;G13,H,H,c-Pr,-], [ZB594;G13,H,H,Ph,-], [ZB595;G13,H,H,OMe,-], [ZB596;G13,H,H,OEt,-], [ZB597;G13,H,H,OPh,-], [ZB598;G13,H,H,CF₃,-], [ZB 599;G13,H,H,F,-], [ZB600;G13,H,H,Cl,-], [ZB601;G13,H,H,Br,-], [ZB602;G13,H,H,CN,-], [ZB603;G13,H,Me,H,-], [ZB604;G13,Me,Me,H,-], [ZB605;G13,E t,Me,H,-], [ZB606;G13,Pr,Me,H,-], [ZB607;G13,i-Pr,Me,H,-], [ZB608;G13,c-Pr,Me,H,-], [ZB609;G13,Ph,Me,H,-], [ZB610;G13,OMe,Me,H,-], [ZB 611;G13,OEt,Me,H,-], [ZB612;G13,OPh,Me,H,-], [ZB613;G13,CF₃,M e,H,-], [ZB614;G13,F,Me,H,-], [ZB615;G13,Cl,Me,H,-], [ZB616;G1 3,Br,Me,H,-], [ZB617;G13,CN,Me,H,-], [ZB618;G13,H,Me,Me,-], [Z B619;G13,H,Me,Et,-], [ZB620;G13,H,Me,Pr,-], [ZB621;G13,H,Me,i-Pr,-], [ZB622;G13,H,Me,c-Pr,-], [ZB623;G13,H,Me,Ph,-], [ZB624;G13,H,Me,OMe,-], [ZB625;G 13,H,Me,OEt,-], [ZB626;G13,H,Me,OPh,-], [ZB627;G13,H,Me,CF₃,-], [ZB628;G13,H,Me,F,-], [ZB629;G13,H,Me,Cl,-], [ZB630;G13,H,M e,Br,-], [ZB631;G13,H,Me,CN,-], [ZB632;G14,H,H,H,-], [ZB633;G1 4,Me,H,H,-], [ZB634;G14,Et,H,H,-], [ZB635;G14,Pr,H,H,-], [ZB63 6;G14,i-Pr,H,H,-], [ZB637;G14,c-Pr,H,H,-], [ZB638;G14,Ph,H,H,-], [ZB639;G14,OMe,H,H,-], [ZB640;G14,OEt,H,H,-], [ZB641;G14,OPh,H,H,-], [ZB642;G14,CF₃,H,H,-], [ZB643;G14,F,H,H,-], [ZB644;G14,Cl,H,H,-], [ZB645;G14,Br,H,H,-], [ZB646;G14,CN,H,H,-], [ZB647;G14,H,Me,H,-], [ZB648;G14,H, Et,H,-], [ZB649;G14,H,Pr,H,-], [ZB650;G14,H,i-Pr,H,-], [ZB651;G14,H,c-Pr,H,-], [ZB652;G14,H,Ph,H,-], [ZB653;G14,H,OMe,H,-], [ZB654;G 14,H,OEt,H,-], [ZB655;G14,H,OPh,H,-], [ZB656;G14,H,CF₃,H,-], [ZB657;G14,H,F,H,-], [ZB658;G14,H,Cl,H,-], [ZB659;G14,H,Br,H,-], [ZB660;G14,H,CN,H,-], [ZB661;G14,H,H,Me,-], [ZB662;G14,H,H, Et,-], [ZB663;G14,H,H,Pr,-], [ZB664;G14,H,H,i-Pr,-], [ZB665;G14,H,H,c-Pr,-], [ZB666;G14,H,H,Ph,-], [ZB667;G14,H,H,OMe,-], [ZB668;G14,H,H,OEt,-], [ZB669;G14,H,H,OPh,-], [ZB670;G14,H,H,CF₃,-], [ZB 671;G14,H,H,F,-], [ZB672;G14,H,H,Cl,-], [ZB673;G14,H,H,Br,-], [ZB674;G14,H,H,CN,-], [ZB675;G15,H,H,H,-], [ZB676;G15,Me,H,H,-], [ZB677;G15,Et,H,H,-], [ZB678;G15,Pr,H,H,-], [ZB679;G15,i-Pr,H,H,-], [ZB680;G15,c-Pr,H,H,-], [ZB681;G15,Ph,H,H,-], [ZB682;G15,OMe,H,H,-], [ZB683;G15,OEt,H,H,-], [ZB684;G15,OPh,H,H,-], [ZB685;G15,CF₃,H,H,-], [ZB686;G15,F,H,H,-], [ZB687;G15,Cl,H,H,-], [ZB688;G15,Br,H,H,-], [ZB689;G15,CN,H,H,-], [ZB690;G15,H,Me,H,-], [ZB691;G15,H, Et,H,-], [ZB692;G15,H,Pr,H,-], [ZB693;G15,H,i-Pr,H,-], [ZB694;G15,H,c-Pr,H,-], [ZB695;G15,H,Ph,H,-], [ZB696;G15,H,OMe,H,-], [ZB697;G 15,H,OEt,H, -], [ZB698;G15,H,OPh,H, -], [ZB699;G15,H,CF₃,H,-], [ZB700;G15,H,F,H,-], [ZB701;G15,H,Cl,H,-], [ZB702;G15,H,Br,H,-], [ZB703;G15,H,CN,H,-], [ZB704;G15,H,H,Me,-], [ZB705;G15,H,H, Et,-], [ZB706;G15,H,H,Pr,-], [ZB707;G15,H,H,i-Pr,-], [ZB708;G15,H,H,c-Pr,-], [ZB709;G15,H,H,Ph,-], [ZB710;G15,H,H,OMe,-], [ZB711;G15,H,H,OEt,-], [ZB712;G15,H,H,OPh,-], [ZB713;G15,H,H,CF₃,-], [ZB 714;G15,H,H,F,-], [ZB715;G15,H,H,Cl,-], [ZB716;G15,H,H,Br,-], [ZB717;G15,H,H,CN,-], [ZB718;G16,H,H,-,-], [ZB719;G16,Me,H,-,-], [ZB720;G16,Et,H,-,-], [ZB721;G16,Pr,H,-,-], [ZB722;G16,i-Pr,H,-,-], [ZB723;G16,c-Pr,H,-,-], [ZB724;G16,Ph,H,-,-], [ZB725;G16,OMe,H,-,-], [ZB726;G16,OEt,H, -, -], [ZB727;G16,OPh,H, -, -], [ZB728;G16,CF₃,H, -,-], [ZB729;G16,F,H,-,-], [ZB730;G16,Cl,H,-,-], [ZB731;G16,Br,H,-,-], [ZB732;G16,CCN,H,-,-], [ZB733;G16,H,Me,-,-], [ZB734;G16,H, Et,-,-], [ZB735;G16,H,Pr,-,-], [ZB736;G16,H,i-Pr,-,-], [ZB737;G16,H,c-Pr,-,-], [ZB738;G16,H,Ph,-,-], [ZB739;G16,H,OMe,-,-], [ZB740;G 16,H,OEt,-,-], [ZB741;G16,H,OPh,-,-], [ZB742;G16,H,CF₃,-,-], [ZB743;G16,H,F,-,-], [ZB744;G16,H,Cl,-,-], [ZB745;G16,H,Br,-,-], [ZB746;G16,H,CN,-,-], [ZB747;G17,H,H,-,-], [ZB748;G17,Me,H, -,-], [ZB749;G17,Et,H,-,-], [ZB750;G17,Pr,H,-,-], [ZB751;G17,i-Pr,H,-,-], [ZB752;G17,c-Pr,H,-,-], [ZB753;G17,Ph,H,-,-], [ZB754;G17,OMe,H,-,-], [ZB755;G17,OEt,H, -, -], [ZB756;G17,OPh,H, -, -], [ZB757;G17,CF₃,H, -,-], [ZB758;G17,F,H,-,-], [ZB759;G17,Cl,H,-,-], [ZB760;G17,Br,H,-,-], [ZB761;G17,CCN,H,-,-], [ZB762;G17,H,Me,-,-], [ZB763;G17,H, Et,-,-], [ZB764;G17,H,Pr,-,-], [ZB765;G17,H,i-Pr,-,-], [ZB766;G17,H,c-Pr,-,-], [ZB767;G17,H,Ph,-,-], [ZB768;G17,H,OMe,-,-], [ZB769;G 17,H,OEt,-,-], [ZB770;G17,H,OPh,-,-], [ZB771;G17,H,CF₃,-,-], [ZB772;G17,H,F,-,-], [ZB773;G17,H,Cl,-,-], [ZB774;G17,H,Br,-,-], [ZB775;G17,H,CN,-,-], [ZB776;G18,H,H,-,-], [ZB777;G18,Me,H, -,-], [ZB778;G18,Et,H,-,-], [ZB779;G18,Pr,H,-,-], [ZB780;G18,i-Pr,H,-,-], [ZB781;G18,c-Pr,H,-,-], [ZB782;G18,Ph,H,-,-], [ZB783;G18,OMe,H,-,-], [ZB784;G18,OEt,H, -, -], [ZB785;G18,OPh,H, -, -], [ZB786;G18,CF₃,H, -,-], [ZB787;G18,F,H,-,-], [ZB788;G18,Cl,H,-,-], [ZB789;G18,Br,H,-,-], [ZB790;G18,CN,H,-,-], [ZB791;G18,H,Me,-,-], [ZB792;G18,H, Et,-,-], [ZB793;G18,H,Pr,-,-], [ZB794;G18,H,i-Pr,-,-], [ZB795;G18,H,c-Pr,-,-], [ZB796;G18,H,Ph,-,-], [ZB797;G18,H,OMe,-,-], [ZB798;G 18,H,OEt,-,-], [ZB799;G18,H,OPh,-,-], [ZB800;G18,H,CF₃,-,-], [ZB801;G18,H,F,-,-], [ZB802;G18,H,Cl,-,-], [ZB803;G18,H,Br,-,-], [ZB804;G18,H,CN,-,-], [ZB805;G19,H,H,-,-], [ZB806;G19,Me,H,-,-], [ZB807;G19,Et,H,-,-], [ZB808;G19,Pr,H,-,-], [ZB809;G19, i-Pr,H,-,-], [ZB810;G19,c-Pr,H,-,-], [ZB811;G19,Ph,H,-,-], [ZB812;G19,OMe,H,-,-], [ZB813;G19,OEt,H, -, -], [ZB814;G19,OPh,H, -, -], [ZB815;G19,CF₃,H, -,-], [ZB816;G19,F,H,-,-], [ZB817;G19,Cl,H,-,-], [ZB818;G19,Br,H,-,-], [ZB819;G19,CN,H,-,-], [ZB820;G19,H,Me,-,-], [ZB821;G19,H, Et,-,-], [ZB822;G19,H,Pr,-,-], [ZB823;G19,H,i-Pr,-,-], [ZB824;G19,H,c-Pr,-,-], [ZB825;G19,H,Ph,-,-], [ZB826;G19,H,OMe,-,-], [ZB827;G 19,H,OEt,-,-], [ZB828;G19,H,OPh,-,-], [ZB829;G19,H,CF₃,-,-], [ZB830;G19,H,F,-,-], [ZB831;G19,H,Cl,-,-], [ZB832;G19,H,Br,-,-], [ZB833;G19,H,CN,-,-], [ZB834;G20,H,H,-,-], [ZB835;G20,Me,H, -,-], [ZB836;G20,Et,H,-,-], [ZB837;G20,Pr,H,-,-], [ZB838;G20,i-Pr,H,-,-], [ZB839;G20,c-Pr,H,-,-], [ZB840;G20,Ph,H,-,-], [ZB841;G20,OMe,H,-,-], [ZB842;G20,OEt,H, -, -], [ZB843;G20,OPh,H, -, -], [ZB844;G20,CF₃,H, -,-], [ZB845;G20,F,H,-,-], [ZB846;G20,Cl,H,-,-], [ZB847;G20,Br,H,-,-], [ZB848;G20,CN,H,-,-], [ZB849;G20,H,Me,-,-], [ZB850;G20,H, Et,-,-], [ZB851;G20,H,Pr,-,-], [ZB852;G20,H,i-Pr,-,-], [ZB853;G20,H,c-Pr,-,-], [ZB854;G20,H,Ph,-,-], [ZB855;G20,H,OMe,-,-], [ZB856;G 20,H,OEt,-,-], [ZB857;G20,H,OPh,-,-], [ZB858;G20,H,CF₃,-,-], [ZB859;G20,H,F,-,-], [ZB860;G20,H,Cl,-,-], [ZB861;G20,H,Br,-,-], [ZB862;G20,H,CN,-,-], [ZB863;G21,H,H,-,-], [ZB864;G21,Me,H, -,-], [ZB865;G21,Et,H,-,-], [ZB866;G21,Pr,H,-,-], [ZB867;G21,i-Pr,H,-,-], [ZB868;G21,c-Pr,H,-,-], [ZB869;G21,Ph,H,-,-], [ZB870;G21,OMe,H,-,-], [ZB871;G21,OEt,H, -,-], [ZB872;G21,OPh,H, -,-], [ZB873;G21,CF₃,H,-,-], [ZB874;G21,F,H,-,-], [ZB875;G21,Cl,H,-,-], [ZB876;G21,Br,H,-,-], [ZB877;G21,CN,H,-,-], [ZB878;G21,H,Me,-,-], [ZB879;G21,H, Et,-,-], [ZB880;G21,H,Pr,-,-], [ZB881;G21,H,i-Pr,-,-], [ZB882;G21,H,c-Pr,-,-], [ZB883;G21,H,Ph,-,-], [ZB884;G21,H,OMe,-,-], [ZB885;G 21,H,OEt,-,-], [ZB886;G21,H,OPh,-,-], [ZB887;G21,H,CF₃,-,-], [ZB888;G21,H,F,-,-], [ZB889;G21,H,Cl,-,-], [ZB890;G21,H,Br,-,-], [ZB891;G21,H,CN,-,-], [ZB892;G22,H,H,-,-], [ZB893;G22,Me,H, -,-], [ZB894;G22,Et,H,-,-], [ZB895;G22,Pr,H,-,-], [ZB896;G22,i-Pr,H,-,-], [ZB897;G22,c-Pr,H,-,-], [ZB898;G22,Ph,H,-,-], [ZB899;G22,OMe,H,-,-], [ZB900;G22,OEt,H,-,-], [ZB901;G22,OPh,H,-,-], [ZB902;G22,CF₃, H,-,-], [ZB903;G22,F,H,-,-], [ZB904;G22,Cl,H,-,-], [ZB905;G22, Br,H,-,-], [ZB906;G22,CN,H,-,-], [ZB907;G22,H,Me,-,-], [ZB908; G22,H,Et,-,-], [ZB909;G22,H,Pr,-,-], [ZB910;G22,H,i-Pr,-,-], [ZB911;G22,H,c-Pr,-,-], [ZB912;G22,H,Ph,-,-], [ZB913;G22,H,OMe,-,-], [ZB914;G 22,H,OEt,-,-], [ZB915;G22,H,OPh,-,-], [ZB916;G22,H,CF₃,-,-], [ZB917;G22,H,F,-,-], [ZB918;G22,H,Cl,-,-], [ZB919;G22,H,Br,-,-], [ZB920;G22,H,CN,-,-], [ZB921;G23,H,H,-,-], [ZB922;G23,Me,H, -,-], [ZB923;G23,Et,H,-,-], [ZB924;G23,Pr,H,-,-], [ZB925;G23,i-Pr,H,-,-], [ZB926;G23,c-Pr,H,-,-], [ZB927;G23,Ph,H,-,-], [ZB928;G23,OMe,H,-,-], [ZB929;G23,OEt,H, -, -], [ZB930;G23,OPh,H, -, -], [ZB931;G23,CF₃,H, -, -], [ZB932;G23,F,H,-,-], [ZB933;G23,Cl,H,-,-], [ZB934;G23,Br,H,-,-], [ZB935;G23,CCN,H,-,-], [ZB936;G23,H,Me,-,-], [ZB937;G23,H, Et,-,-], [ZB938;G23,H,Pr,-,-], [ZB939;G23,H,i-Pr,-,-], [ZB940;G23,H,c-Pr,-,-], [ZB941;G23,H,Ph,-,-], [ZB942;G23,H,OMe,-,-], [ZB943;G 23,H,OEt,-,-], [ZB944;G23,H,OPh,-,-], [ZB945;G23,H,CF₃,-,-], [ZB946;G23,H,F,-,-], [ZB947;G23,H,Cl,-,-], [ZB948;G23,H,Br,-,-], [ZB949;G23,H,CN,-,-], [ZB950;G24,H,H,-,-], [ZB951;G24,Me,H, -,-], [ZB952;G24,Et,H,-,-], [ZB953;G24,Pr,H,-,-], [ZB954;G24,i-Pr,H,-,-], [ZB955;G24,c-Pr,H,-,-], [ZB956;G24,Ph,H,-,-], [ZB957;G24,OMe,H,-,-], [ZB958;G24,OEt,H, -, -], [ZB959;G24,OPh,H, -, -], [ZB960;G24,CF₃,H, -,-], [ZB961;G24,F,H,-,-], [ZB962;G24,Cl,H,-,-], [ZB963;G24,Br,H,-,-], [ZB964;G24,CN,H,-,-], [ZB965;G24,H,Me,-,-], [ZB966;G24,H, Et,-,-], [ZB967;G24,H,Pr,-,-], [ZB968;G24,H,i-Pr,-,-], [ZB969;G24,H,c-Pr,-,-], [ZB970;G24,H,Ph,-,-], [ZB971;G24,H,OMe,-,-], [ZB972;G 24,H,OEt,-,-], [ZB973;G24,H,OPh,-,-], [ZB974;G24,H,CF₃,-,-], [ZB975;G24,H,F,-,-], [ZB976;G24,H,Cl,-,-], [ZB977;G24,H,Br,-,-], [ZB978;G24,H,CN,-,-], [ZB979;G25,H,H,H,-], [ZB980;G25,H,Me, H,-], [ZB981;G25,H,Et,H,-], [ZB982;G25,H,Pr,H,-], [ZB983;G25,H,i-Pr,H,-], [ZB984;G25,H,c-Pr,H,-], [ZB985;G25,H,Ph,H,-], [ZB986;G25,H,OMe,H,-], [ZB987;G 25,H,OEt,H, -], [ZB988;G25,H,OPh,H, -], [ZB989;G25,H,CF₃,H,-], [ZB990;G25,H,F,H,-], [ZB991;G25,H,Cl,H,-], [ZB992;G25,H,Br,H,-], [ZB993;G25,H,CN,H,-], [ZB994;G25,H,H,Me,-], [ZB995;G25,H,H, Et,-], [ZB996;G25,H,H,Pr,-], [ZB997;G25,H,H,i-Pr,-], [ZB998;G25,H,H,c-Pr,-], [ZB999;G25,H,H,Ph,-], [ZB1000;G25,H,H,OMe,-], [ZB1001;G 25,H,H,OEt,-], [ZB1002;G25,H,H,OPh,-], [ZB1003;G25,H,H,CF₃,-], [ZB1004;G25,H,H,F,-], [ZB1005;G25,H,H,Cl,-], [ZB1006;G25,H,H,Br,-], [ZB1007;G25,H,H,CN,-], [ZB1008;G25,Me,H,H,-], [ZB1009; G25,Me,Me,H,-], [ZB1010;G25,Me,Et,H,-], [ZB1011;G25,Me,Pr,H,-], [ZB1012;G25,Me,i-Pr,H,-], [ZB1013;G25,Me,c-Pr,H,-], [ZB1014;G25,Me,Ph,H,-], [ZB1015;G25,Me,OMe,H,-], [ZB1 016;G25,Me,OEt,H,-], [ZB1017;G25,Me,OPh,H,-], [ZB1018;G25,Me, CF₃,H,-], [ZB1019;G25,Me,F,H,-], [ZB1020;G25,Me,Cl,H,-], [ZB10 21;G25,Me,Br,H,-], [ZB1022;G25,Me,CN,H,-], [ZB1023;G25,Me,H,M e,-], [ZB1024;G25,Me,H,Et,-], [ZB1025;G25,Me,H,Pr,-], [ZB1026; G25,Me,H,i-Pr,-], [ZB1027;G25,Me,H,c-Pr,-], [ZB1028;G25,Me,H,Ph,-], [ZB1029;G25,Me,H,OMe,-], [ZB103 0;G25,Me,H,OEt,-], [ZB1031;G25,Me,H,OPh,-], [ZB1032;G25,Me,H, CF₃,-], [ZB1033;G25,Me,H,F,-], [ZB1034;G25,Me,H,Cl,-], [ZB1035;G25,Me,H,Br,-], [ZB1036;G25,Me,H,CN,-], [ZB1037;G26,H,H,H,-], [ZB1038;G26,H,Me,H,-], [ZB1039;G26,H,Et,H,-], [ZB1040;G26,H, Pr,H,-], [ZB1041;G26,H,i-Pr,H,-], [ZB1042;G26,H,c-Pr,H,-], [ZB1043;G26,H,Ph,H,-], [ZB1044;G26,H,OMe,H,-], [ZB104 5;G26,H,OEt,H,-], [ZB1046;G26,H,OPh,H,-], [ZB1047;G26,H,CF₃,H,-], [ZB1048;G26,H,F,H,-], [ZB1049;G26,H,Cl,H,-], [ZB1050;G26, H,Br,H,-], [ZB1051;G26,H,CN,H,-], [ZB1052;G26,H,H,Me,-], [ZB10 53;G26,H,H,Et,-], [ZB1054;G26,H,H,Pr,-], [ZB1055;G26,H,H,i-Pr,-], [ZB1056;G26,H,H,c-Pr,-], [ZB1057;G26,H,H,Ph,-], [ZB1058;G26,H,H,OMe,-], [ZB1059; G26,H,H,OEt,-], [ZB1060;G26,H,H,OPh,-], [ZB1061;G26,H,H,CF₃,-], [ZB1062;G26,H,H,F,-], [ZB1063;G26,H,H,Cl,-], [ZB1064;G26,H, H,Br,-], [ZB1065;G26,H,H,CN,-], [ZB1066;G26,Me,H,H,-], [ZB1067;G26,Me,Me,H,-], [ZB1068;G26,Me,Et,H,-], [ZB1069;G26,Me,Pr,H,-], [ZB1070;G26,Me,i-Pr,H,-], [ZB1071;G26,Me,c-Pr,H,-], [ZB1072;G26,Me,Ph,H,-], [ZB1073;G26,Me,OMe,H,-], [ZB1 074;G26,Me,OEt,H,-], [ZB1075;G26,Me,OPh,H,-], [ZB1076;G26,Me, CF₃,H,-], [ZB1077;G26,Me,F,H,-], [ZB1078;G26,Me,Cl,H,-], [ZB10 79;G26,Me,Br,H,-], [ZB1080;G26,Me,CN,H,-], [ZB1081;G26,Me,H,M e,-], [ZB1082;G26,Me,H,Et,-], [ZB1083;G26,Me,H,Pr,-], [ZB1084; G26,Me,H,i-Pr,-], [ZB1085;G26,Me,H,c-Pr,-], [ZB1086;G26,Me,H,Ph,-], [ZB1087;G26,Me,H,OMe,-], [ZB108 8;G26,Me,H,OEt,-], [ZB1089;G26,Me,H,OPh,-], [ZB1090;G26,Me,H, CF₃,-], [ZB1091;G26,Me,H,F,-], [ZB1092;G26,Me,H,Cl,-], [ZB1093;G26,Me,H,Br,-], [ZB1094;G26,Me,H,CN,-], [ZB1095;G27,H,H,H,-],[ZB1096;G27,Me,H,H,-], [ZB1097;G27,Et,H,H,-], [ZB1098;G27,Pr,H,H,-], [ZB1099;G27,i-Pr,H,H,-], [ZB1100;G27,c-Pr,H,H,-], [ZB1101;G27,Ph,H,H,-], [ZB1102;G27,OMe,H,H,-], [ZB1 103;G27,OEt,H,H,-], [ZB1104;G27,OPh,H,H,-], [ZB1105;G27,CF₃,H,H,-], [ZB1106;G27,F,H,H,-], [ZB1107;G27,Cl,H,H,-], [ZB1108;G2 7,Br,H,H,-], [ZB1109;G27,CN,H,H,-], [ZB1110;G27,H,Me,H,-], [ZB 1111;G27,H,Et,H,-], [ZB1112;G27,H,Pr,H,-], [ZB1113;G27,H,i-Pr,H,-], [ZB1114;G27,H,c-Pr,H,-], [ZB1115;G27,H,Ph,H,-], [ZB1116;G27,H,OMe,H,-], [ZB111 7;G27,H,OEt,H,-], [ZB1118;G27,H,OPh,H,-], [ZB1119;G27,H,CF₃,H,-], [ZB1120;G27,H,F,H,-], [ZB1121;G27,H,Cl,H,-], [ZB1122;G27, H,Br,H,-], [ZB1123;G27,H,CN,H,-], [ZB1124;G27,H,H,Me,-], [ZB11 25;G27,H,H,Et,-], [ZB1126;G27,H,H,Pr,-], [ZB1127;G27,H,H,i-Pr,-], [ZB1128;G27,H,H,c-Pr,-], [ZB1129;G27,H,H,Ph,-], [ZB1130;G27,H,H,OMe,-], [ZB1131; G27,H,H,OEt,-], [ZB1132;G27,H,H,OPh,-], [ZB1133;G27,H,H,CF₃,-], [ZB1134;G27,H,H,F,-], [ZB1135;G27,H,H,Cl,-], [ZB1136;G27,H, H,Br,-], [ZB1137;G27,H,H,CN,-], [ZB1138;G28,H,H,-,-], [ZB1139; G28,Me,H,-,-], [ZB1140;G28,Cl,H,-,-], [ZB1141;G28,CF₃,H,-,-], [ZB1142;G28,H,Me,-,-], [ZB1143;G28,H,Cl,-,-], [ZB1144;G28,H,C F₃,-,-], [ZB1145;G28,H,SH,-,-], [ZB1146;G29,H,H,H,-], [ZB1147; G29,H,Me,H,-], [ZB1148;G29,H,Et,H,-], [ZB1149;G29,H,Pr,H,-], [ZB1150;G29,H,i-Pr,H,-], [ZB1151;G29,H,c-Pr,H,-], [ZB1152;G29,H,Ph,H,-], [ZB1153;G29,H,OMe,H,-], [ZB115 4;G29,H,OEt,H,-], [ZB1155;G29,H,OPh,H,-], [ZB1156;G29,H,CF₃,H,-], [ZB1157;G29,H,F,H,-], [ZB1158;G29,H,Cl,H,-], [ZB1159;G29, H,Br,H,-], [ZB1160;G29,H,CN,H,-], [ZB1161;G29,H,H,Me,-], [ZB11 62;G29,H,H,Et,-], [ZB1163;G29,H,H,Pr,-], [ZB1164;G29,H,H,i-Pr,-], [ZB1165;G29,H,H,c-Pr,-], [ZB1166;G29,H,H,Ph,-], [ZB1167;G29,H,H,OMe,-], [ZB1168; G29,H,H,OEt,-], [ZB1169;G29,H,H,OPh,-], [ZB1170;G29,H,H,CF₃,-], [ZB1171;G29,H,H,F,-], [ZB1172;G29,H,H,Cl,-], [ZB1173;G29,H, H,Br,-], [ZB1174;G29,H,H,CN,-], [ZB1175;G29,Me,H,H,-], [ZB1176;G29,Me,Me,H,-], [ZB1177;G29,Me,Et,H,-], [ZB1178;G29,Me,Pr,H,-], [ZB1179;G29,Me,i-Pr,H,-], [ZB1180;G29,Me,c-Pr,H,-], [ZB1181;G29,Me,Ph,H,-], [ZB1182;G29,Me,OMe,H,-], [ZB1 183;G29,Me,OEt,H,-], [ZB1184;G29,Me,OPh,H,-], [ZB1185;G29,Me, CF₃,H,-], [ZB1186;G29,Me,F,H,-], [ZB1187;G29,Me,Cl,H,-], [ZB11 88;G29,Me,Br,H,-], [ZB1189;G29,Me,CN,H,-], [ZB1190;G29,Me,H,M e,-], [ZB1191;G29,Me,H,Et,-], [ZB1192;G29,Me,H,Pr,-], [ZB1193; G29,Me,H,i-Pr,-], [ZB1194;G29,Me,H,c-Pr,-], [ZB1195;G29,Me,H,Ph,-], [ZB1196;G29,Me,H,OMe,-], [ZB119 7;G29,Me,H,OEt,-], [ZB1198;G29,Me,H,OPh,-], [ZB1199;G29,Me,H, CF₃,-], [ZB1200;G29,Me,H,F,-], [ZB1201;G29,Me,H,Cl,-], [ZB1202;G29,Me,H,Br,-], [ZB1203;G29,Me,H,CN,-], [ZB1204;G30,H,H,H,-], [ZB1205;G30,Me,H,H,-], [ZB1206;G30,Et,H,H,-], [ZB1207;G30,Pr,H,H,-], [ZB1208;G30,i-Pr,H,H,-], [ZB1209;G30,c-Pr,H,H,-], [ZB1210;G30,Ph,H,H,-], [ZB1211;G30,OMe,H,H,-], [ZB1 212;G30,OEt,H,H,-], [ZB1213;G30,OPh,H,H,-], [ZB1214;G30,CF₃,H,H,-], [ZB1215;G30,F,H,H,-], [ZB1216;G30,Cl,H,H,-], [ZB1217;G3 0,Br,H,H,-], [ZB1218;G30,CN,H,H,-], [ZB1219;G30,H,Me,H,-], [ZB 1220;G30,H,Et,H,-], [ZB1221;G30,H,Pr,H,-], [ZB1222;G30,H,i-Pr,H,-], [ZB1223;G30,H,c-Pr,H,-], [ZB1224;G30,H,Ph,H,-], [ZB1225;G30,H,OMe,H,-], [ZB122 6;G30,H,OEt,H,-], [ZB1227;G30,H,OPh,H,-], [ZB1228;G30,H,CF₃,H,-], [ZB1229;G30,H,F,H,-], [ZB1230;G30,H,Cl,H,-], [ZB1231;G30, H,Br,H,-], [ZB1232;G30,H,CN,H,-], [ZB1233;G30,H,H,Me,-], [ZB12 34;G30,H,H,Et,-], [ZB1235;G30,H,H,Pr,-], [ZB1236;G30,H,H,i-Pr,-], [ZB1237;G30,H,H,c-Pr,-], [ZB1238;G30,H,H,Ph,-], [ZB1239;G30,H,H,OMe,-], [ZB1240; G30,H,H,OEt,-], [ZB1241;G30,H,H,OPh,-], [ZB1242;G30,H,H,CF₃,-], [ZB1243;G30,H,H,F,-], [ZB1244;G30,H,H,Cl,-], [ZB1245;G30,H, H,Br,-], [ZB1246;G30,H,H,CN,-], [ZB1247;G30,H,Oi-Pr,H,-], [ZB1248;G30,H,Oc-Pr,H,-], [ZB1249;G30,H,Ot-Bu,H,-], [ZB1250;G30,H,OCF₃,H,-], [ZB1251;G30,H,OCHF₂,H, -], [Z B1252;G30,H,OPr,H,-], [ZB1253;G30,H,OCH₂Ph,H,-], [ZB1253;G30, H,OCH₂-i-Pr,H,-], [ZB1253;G30,H,OCH₂-c-Pr,H,-], [ZB1253;G30,H,OCH₂-t-Bu,H,-], [ZB1254;G31,H,-,-,-], [ZB1255;G31,Me,-,-,-], [ZB1256; G31,Et,-,-,-], [ZB1257;G31,Pr,-,-,-], [ZB1258;G31,i-Pr,-,-,-], [ZB1259;G31,c-Pr,-,-,-], [ZB1260;G31,Ph,-,-,-], [ZB1261;G31,OMe,-,-,-], [ZB1 262;G31,OEt,-,-,-], [ZB1263;G31,OPh,-,-,-], [ZB1264;G31,CF₃,-,-,-], [ZB1265;G31,F,-,-,-], [ZB1266;G31,Cl,-,-,-], [ZB1267;G3 1,Br,-,-,-], [ZB1268;G31, CN,-,-,-], [ZB1269;G31, t-Bu,-,-,-], [ZB1270;G32,H,-,-,-], [ZB1271;G32,Me,-,-,-], [ZB127 2;G32,Et,-,-,-], [ZB1273;G32,Pr,-,-,-], [ZB1274;G32,i-Pr,-,-,-], [ZB1275;G32,c-Pr,-,-,-], [ZB1276;G32,Ph,-,-,-], [ZB1277;G32,OMe,-,-,-], [ZB1 278;G32,OEt,-,-,-], [ZB1279;G32,OPh,-,-,-], [ZB1280;G32,CF₃,-,-,-], [ZB1281;G32,F,-,-,-], [ZB1282;G32,Cl,-,-,-], [ZB1283;G3 2,Br,-,-,-], [ZB1284;G32,CN,-,-,-], [ZB1285;G32,t-Bu,-,-,-], [ZB1286;G33,H,-,-,-], [ZB1287;G33,Me,-,-,-], [ZB128 8;G33,Et,-,-,-], [ZB1289;G33,Pr,-,-,-], [ZB1290;G33,i-Pr,-,-,-], [ZB1291;G33,c-Pr,-,-,-], [ZB1292;G33,Ph,-,-,-], [ZB1293;G33,OMe,-,-,-], [ZB1 294;G33,OEt,-,-,-], [ZB1295;G33,OPh,-,-,-], [ZB1296;G33,CF₃,-,-,-], [ZB1297;G33,F,-,-,-], [ZB1298;G33,Cl,-,-,-], [ZB1299;G3 3,Br,-,-,-], [ZB1300;G33,CN,-,-,-], [ZB1301;G33,t-Bu,-,-,-], [ZB1302;G34,H,-,-,-], [ZB1303;G34,Me,-,-,-], [ZB130 4;G34,Et,-,-,-], [ZB1305;G34,Pr,-,-,-], [ZB1306;G34,i-Pr,-,-,-], [ZB1307;G34,c-Pr,-,-,-], [ZB1308;G34,Ph,-,-,-], [ZB1309;G34,OMe,-,-,-], [ZB1 310;G34,OEt,-,-,-], [ZB1311;G34,OPh,-,-,-], [ZB1312;G34,CF₃,-,-,-], [ZB1313;G34,F,-,-,-], [ZB1314;G34,Cl,-,-,-], [ZB1315;G3 4,Br,-,-,-], [ZB1316;G34,CN,-,-,-], [ZB1317;G34,t-Bu,-,-,-], [ZB1318;G12,t-Bu,H,H,-], [ZB1319;G12,CH(Me)Et,H,H,-], [ZB1320;G12,CH(Et)₂,H,H,-], [ZB1321;G12,CH₂(i-Pr),H,H,-], [ZB1322;G12,C(Me)=CH(Me),H,H,-], [ZB1323;G12,C(Me)=CH₂,H,H,-], [ZB1324;G12,CH═CH(Me),H,H,-], [ZB1325;G12,CH═CH₂,H,H,-], [ZB1326;G12,CC(c-Pr),H,H,-], [ZB1327;G12,CC(Me),H,H,-], [ZB1328;G12,CHF₂,H,H,-], [ZB1329;G12,CF₂Me,H,H,-], [ZB1330;G12,CF₂CF₃,H,H,-], [ZB1331;G12,C(CF₃)CH₂,H,H,-], [ZB1332;G12,CF₂Cl,H,H,-], [ZB1333;G12,C H(Me)CF₃,H,H,-], [ZB1334;G12,c-Bu,H,H,-], [ZB1335;G12,c-Pen,H,H,-], [ZB1336;G12,c-Hex,H,H,-], [ZB1337;G12,1-Me-(c-Pr),H,H,-], [ZB1338;G12,1-F-(c-Pr),H,H,-], [ZB1339;G12,2,2-F₂-(c-Pr),H,H,-], [ZB1340;G12,I,H,H, -], [ZB1341;G12,CH₂CN,H,H, -], [Z B1342;G12,CH₂OMe,H,H,-], [ZB1343;G12,CH₂SMe,H,H,-], [ZB1344;G 12,c-hexen-1-yl,H,H,-], [ZB1345;G12,OPr,H,H,-], [ZB1346;G12,O(i-Pr),H,H,-], [ZB1347;G12,OCHF₂,H,H,-], [ZB1348;G12,SCF₃,H,H,-],[ZB1349;G12,C(Me)=NOMe,H,H,-], [ZB1350;G12,H,t-Bu,H,-], [ZB1351;G12,H,c-Bu,H,-], [ZB1352;G12,H,c-Pen,H,-], [ZB1353;G12,H,CCH,H,-], [ZB1354;G12,H,CH₂OMe,H,-], [ZB1355;G12,H,I,H,-], [ZB1356;G12,H,S(i-Pr),H,-], [ZB1357;G12,Me,Me,H, -], [ZB1358;G12,Pr,Me,H, -], [ZB1 359;G12,i-Pr,Me,H,-], [ZB1360;G12,CHF₂,Me,H,-], [ZB1361;G12,CF₃,Me,H,-],[ZB1362;G12,c-Pr,Me,H,-], [ZB1363;G12,c-Hex,Me,H,-], [ZB1364;G12,OMe,Me,H,-], [ZB1365;G12,O(i-Pr),Me,H,-], [ZB1366;G12,F,Me,H,-], [ZB1367;G12,Cl,Me,H,-], [Z B1368;G12,Br,Me,H,-], [ZB1369;G12,i-Pr,F,H,-], [ZB1370;G12,Me,F,H,-], [ZB1371;G12,Pr,F,H,-], [ZB13 72;G12,CHF₂,F,H,-], [ZB1373;G12,CF₃,F,H,-], [ZB1374;G12,c-Pr,F,H,-], [ZB1375;G12,c-Hex,F,H,-], [ZB1376;G12,OMe,F,H,-], [ZB1377;G12,O(i-Pr),F,H,-], [ZB1378;G12,F,F,H,-], [ZB1379;G12,Cl,F,H,-], [ZB13 80;G12,Br,F,H,-], [ZB1381;G12,Pr,Cl,H,-], [ZB1382;G12,i-Pr,Cl,H,-], [ZB1383;G12,c-Hex,Cl,H,-], [ZB1384;G12,Cl,Cl,H,-], [ZB1385;G12,O(i-Pr),Cl,H,-], [ZB1386;G12,Me,Cl,H,-], [ZB1387;G12,CHF₂,Cl,H, -], [ZB1388;G12,CF₃,Cl,H,-], [ZB1389;G12,c-Pr,Cl,H,-], [ZB1390;G12,OMe,Cl,H,-], [ZB1391;G12,F,Cl,H,-], [Z B1392;G12,Br,Cl,H,-], [ZB1393;G12,Me,Br,H,-], [ZB1394;G12,c-Hex,Br,H,-], [ZB1395;G12,Pr,Br,H,-], [ZB1396;G12,i-Pr,Br,H,-], [ZB1397;G12,CHF₂,Br,H,-], [ZB1398;G12,CF₃,Br,H,-], [ZB1399;G12,c-Pr,Br,H,-], [ZB1400;G12,OMe,Br,H,-], [ZB1401;G12,O(i-Pr),Br,H,-], [ZB1402;G12,F,Br,H,-], [ZB1403;G12,Cl,Br,H,-], [Z B1404;G12,Br,Br,H,-], [ZB1405;G22,H,Bu,-,-], [ZB1406;G22,H,CH₂(i-Pr),-,-], [ZB1407;G22,H,c-Bu,-,-], [ZB1408;G22,H,c-Pen,-,-], [ZB1409;G22,H,c-Hex,-,-], [ZB1410;G22,H,c-hexen-1-yl,-,-], [ZB1411;G22,H,CHF₂,-,-], [ZB1412;G22,H,CF₂CF₃,-, -], [Z B1413;G22,H,CH═CH₂,-,-], [ZB1414;G22,H,C(Me)=CH₂,-,-], [ZB141 5;G22,H,CC(c-Pr),-,-], [ZB1416;G22,H,CC(i-Pr),-,-], [ZB1417;G22,H,CC(Me),-,-], [ZB1418;G22,H,CH═NOMe,-,-], [ZB1419;G22,H,C(Me)=NOMe,-,-], [ZB1420;G22,H,CH₂O(i-Pr),-,-], [ZB1421;G22,H,CH₂OEt, -, -], [ZB1422;G22,H,CH₂OPh, -, -], [ZB1423;G22,Me,H,-,-], [ZB1424;G22,Et,H,-,-], [ZB1425;G22,P r,H,-,-], [ZB1426;G22,Bu,H,-,-], [ZB1427;G22,CH₂(i-Pr),H,-, -], [ZB1428;G22,c-Bu,H, -, -], [ZB1429;G22,c-Pen,H,-,-], [ZB1430;G22,c-Hex,H,-,-], [ZB1431;G22,c-hexen-1-yl,H,-,-], [ZB1432;G22,CHF₂,H,-,-], [ZB1433;G22,CF₂CF₃,H, -,-], [ZB1434;G22,CN,H,-,-], [ZB1435;G22,OMe,H,-,-], [ZB1436;G22,CH ═CH₂,H,-, -], [ZB1437;G22,C(Me)=CH₂,H,-, -], [ZB1438;G22,CC(c-Pr),H,-, -], [ZB1439;G22,CC(i-Pr),H,-,-], [ZB1440;G22,CC(Me),H,-,-], [ZB1441;G22,CH═NOMe,H, -,-], [ZB1442;G22,C(Me)=NOMe,H,-,-], [ZB1443;G22,CH₂O(i-Pr),H,-,-], [ZB1444;G22,CH₂OEt,H,-,-], [ZB1445;G22,CH₂OPh,H,-,-], [ZB1446;G22,Me,Me,-,-], [ZB1447;G22,Me,Et,-,-], [ZB1448;G 22,Me,i-Pr,-,-], [ZB1449;G22,Me,c-Pr,-,-], [ZB1450;G22,Me,c-Hex,-,-], [ZB1451;G22,Me,CHF₂,-,-], [ZB1452;G22,Me,CF₃,-,-], [ZB1453;G22,Me,F,-,-], [ZB1454;G22,Me,Cl,-,-], [ZB1455;G22,Me, Br,-,-], [ZB1456;G22,Cl,Me,-,-], [ZB1457;G22,Cl,Et,-,-], [ZB14 58;G22,Cl,i-Pr,-,-], [ZB1459;G22,Cl,c-Pr,-,-], [ZB1460;G22,Cl,c-Hex,-,-], [ZB1461;G22,Cl,CHF₂,-,-], [ZB1462;G22,Cl,CF₃,-,-], [ZB1463;G22,Cl,F,-,-], [ZB1464;G22,Cl,Cl,-,-], [ZB1465;G22,Cl, Br,-,-], [ZB1466;G22,F,Me,-,-], [ZB1467;G22,F,Et,-,-], [ZB1468;G22,F,i-Pr,-,-], [ZB1469;G22,F,c-Pr,-,-], [ZB1470;G22,F,c-Hex,-,-], [ZB1471;G22,F,CHF₂,-, -], [ZB1472;G22,F,CF₃,-,-], [ZB 1473;G22,F,F,-,-], [ZB1474;G22,F,Cl,-,-], [ZB1475;G22,F,Br,-,-], [ZB1476;G35,Me,H, -, -], [ZB1477;G35,Et,H, -, -], [ZB1478;G35, Pr,H,-,-], [ZB1479;G35,Bu,H,-,-], [ZB1480;G35,i-Pr,H,-,-], [ZB1481;G35,C(Me)=CH₂,H,-,-], [ZB1482;G35,CHF₂,H,-,-], [ZB1483;G35,CF₃,H,-,-], [ZB1484;G35,c-Pr,H,-,-], [ZB1485;G35,c-Pen,H,-,-], [ZB1486;G35,c-Hex,H,-,-], [ZB1487;G35,C(Me)=NOH,H,-,-], [ZB1488;G35,C(Me)=N OEt,H,-,-], [ZB1489;G35,F,H,-,-], [ZB1490;G35,Cl,H,-,-], [ZB14 91;G35,Br,H,-,-], [ZB1492;G35,Me,Cl,-,-], [ZB1493;G35,Et,Cl,-,-], [ZB1494;G35,Pr,Cl,-,-], [ZB1495;G35,Bu,Cl,-,-], [ZB1496;G 35,i-Pr,Cl,-,-], [ZB1497;G35,C(Me)=CH₂,Cl,-,-] [ZB1498;G35,CHF₂,Cl,-,-], [ZB1499;G35,CF₃,Cl, -, -], [ZB1500;G35,c-Pr,Cl,-,-], [ZB1501;G35,c-Pen,Cl,-,-], [ZB1502;G35,c-Hex,Cl,-,-], [ZB1503;G35,C(Me)=NOH,Cl,-,-], [ZB1504;G35,C(Me)=NOEt,Cl,-,-], [ZB1505;G35,F,Cl,-,-], [ZB1506;G35,Cl,Cl,-,-], [ZB1507;G35,Br,Cl,-,-], [ZB1508;G35,Me,Br,-,-], [ZB1509;G35,E t,Br,-,-], [ZB1510;G35,Pr,Br,-,-], [ZB1511;G35,Bu,Br,-,-], [ZB 1512;G35,i-Pr,Br,-,-], [ZB1513;G35,C(Me)=CH₂,Br,-,-], [ZB1514;G35,CHF₂,B r,-,-], [ZB1515;G35,CF₃,Br,-,-], [ZB1516;G35,c-Pr,Br,-,-], [ZB1517;G35,c-Pen,Br,-,-], [ZB1518;G35,c-Hex,Br,-,-], [ZB1519;G35,C(Me)=NOH,Br,-,-], [ZB1520;G35,C(Me)=NOEt,Br,-,-], [ZB1521;G35,F,Br,-,-], [ZB1522;G35,Cl,Br,-,-], [ZB1523;G35,Br,Br,-,-], [ZB1524;G35,Me,Me,-,-], [ZB1525;G35,E t,Me,-,-], [ZB1526;G35,Pr,Me,-,-], [ZB1527;G35,Bu,Me,-,-], [ZB 1528;G35,i-Pr,Me,-,-], [ZB1529;G35,C(Me)=CH₂,Me,-,-], [ZB1530;G35,CHF₂,M e,-,-], [ZB1531;G35,CF₃,Me,-,-], [ZB1532;G35,c-Pr,Me,-,-], [ZB1533;G35,c-Pen,Me,-,-], [ZB1534;G35,c-Hex,Me,-,-], [ZB1535;G35,C(Me)=NOH,Me,-,-], [ZB1536;G35,C(Me)=NOEt,Me,-,-], [ZB1537;G35,F,Me,-,-], [ZB1538;G35,Cl,Me,-,-], [ZB1539;G35,Br,Me,-,-], [ZB1540;G22,C≡C-Me,H,-,-], [ZB1541;G22,C≡C-Et,H,-,-], [ZB1542;G22,C≡C-n-Pr,H,-,-], [ZB1543;G23,C≡C-i-Pr,H,-,-], [ZB1544;G22,C≡C-n-Bu,H,-,-], [ZB1545;G22,C≡C-t-Bu,H,-,-], [ZB1546;G23,C≡C-i-Bu,H,-,-], [ZB1547;G22,C≡C-c-Bu,H,-,-]. [ZB1548;G22,C≡C-c-Pen,H,-,-], [ZB1549;G22,C≡C-c-Hex,H,-,-], [ZB1550;G22,C≡C-Ph,H,-,-], [ZB1551;G22,H,C≡C-Me,-,-], [ZB1552;G22,H,C≡C-Et,-,-], [ZB1553;G22,H,C≡C-n-Pr,-,-], [ZB1554;G22,H,C≡C-i-Pr,-,-], [ZB1555;G22,H,C≡C-n-Bu,-,-], [ZB1556;G22,H,C≡C-t-Bu,-,-], [ZB1557;G22,H,C≡C-i-Bu,-,-], [ZB1558;G22,H,C≡C-c-Bu,-,-], [ZB1559;G22,H,C≡C-c-Pen,-,-], [ZB1560;G22,H,C≡C-c-Hex,-,-], [ZB1561;G22,H,C≡C-Ph,-,-], [ZB1562;G22,H,C≡CH,-,-]

A compound according to Compound (1J) wherein R¹ represents a chlorine atom, Q represents Q3, R³ represents a methoxy group, and a combination of structure of G and substituents R^{X7}, R^{X8}, R^{X9} and R^{X10} applied to the structure of G represents any combination described in Combination B (hereinafter, referred to as Compound group SX387).

A compound according to Compound (1J) wherein R¹ represents a methyl group, Q represents Q4, and a combination of structure of G and substituents R^{X7}, R^{X8}, R^{X9} and R^{X10} applied to the structure of G represents any combination described in Combination B (hereinafter, referred to as Compound group SX388).

A compound according to Compound (1J) wherein R¹ represents a chlorine atom, Q represents Q4, and a combination of structure of G and substituents R^{X7}, R^{X8}, R^{X9} and R^{X10} applied to the structure of G represents any combination described in Combination B (hereinafter, referred to as Compound group SX389).

A compound according to Compound (1J) wherein R¹ represents a methyl group, Q represents Q5, and a combination of structure of G and substituents R^{X7}, R^{X8}, R^{X9} and R^{X10} applied to the structure of G represents any combination described in Combination B (hereinafter, referred to as Compound group SX390).

A compound according to Compound (1J) wherein R¹ represents a chlorine atom, Q represents Q5, and a combination of structure of G and substituents R^{X7}, R^{X8}, R^{X9} and R^{X10} applied to the structure of G represents any combination described in Combination B (hereinafter, referred to as Compound group SX391).

A compound according to Compound (1J) wherein R¹ represents a methyl group, Q represents Q6, and a combination of structure of G and substituents R^{X7}, R^{X8}, R^{X9} and R^{X10} applied to the structure of G represents any combination described in Combination B (hereinafter, referred to as Compound group SX392).

A compound according to Compound (1J) wherein R¹ represents a chlorine atom, Q represents Q6, and a combination of structure of G and substituents R^{X7}, R^{X8}, R^{X9} and R^{X10} applied to the structure of G represents any combination described in Combination B (hereinafter, referred to as Compound group SX393).

A compound according to Compound (1J) wherein R¹ represents a methyl group, Q represents Q7, L² represents an oxygen atom, and a combination of structure of G and substituents R^{X7}, R^{X8}, R^{X9} and R^{X10} applied to the structure of G represents any combination described in Combination B (hereinafter, referred to as Compound group SX394).

A compound according to Compound (1J) wherein R¹ represents a chlorine atom, Q represents Q7, L² represents an oxygen atom, and a combination of structure of G and substituents R^{X7}, R^{X8}, R^{X9} and R^{X10} applied to the structure of G represents any combination described in Combination B (hereinafter, referred to as Compound group SX395).

A compound according to Compound (1J) wherein R¹ represents a methyl group, Q represents Q7, L² represents NH, and a combination of structure of G and substituents R^{X7}, R^{X8}, R^{X9} and R^{X10} applied to the structure of G represents any combination described in Combination B (hereinafter, referred to as Compound group SX396).

A compound according to Compound (1J) wherein R¹ represents a chlorine atom, Q represents Q7, L² represents NH, and a combination of structure of G and substituents R^{X7}, R^{X8}, R^{X9} and R^{X10} applied to the structure of G represents any combination described in Combination B (hereinafter, referred to as Compound group SX397).

A compound according to Compound (1J) wherein R¹ represents a methyl group, Q represents Q8A, and a combination of structure of G and substituents R^{X7}, Rx, R^{X9} and R^{X10} applied to the structure of G represents any combination described in Combination B (hereinafter, referred to as Compound group SX398).

A compound according to Compound (1J) wherein R¹ represents a chlorine atom, Q represents Q8A, and a combination of structure of G and substituents R^{X7}, Rx, R^{X9} and R^{X10} applied to the structure of G represents any combination described in Combination B (hereinafter, referred to as Compound group SX399).

A compound according to Compound (1J) wherein R¹ represents a methyl group, Q represents Q8D, and a combination of structure of G and substituents R^{X7}, Rx, R^{X9} and R^{X10} applied to the structure of G represents any combination described in Combination B (hereinafter, referred to as Compound group SX400).

A compound according to Compound (1J) wherein R¹ represents a chlorine atom, Q represents Q8D, and a combination of structure of G and substituents R^{X7}, Rx, R^{X9} and R^{X10} applied to the structure of G represents any combination described in Combination B (hereinafter, referred to as Compound group SX401).

A compound according to Compound (1J) wherein R¹ represents a methyl group, Q represents Q8J, and a combination of structure of G and substituents R^{X7}, R^{X8}, R^{X9} and R^{X10} applied to the structure of G represents any combination described in Combination B (hereinafter, referred to as Compound group SX402).

A compound according to Compound (1J) wherein R¹ represents a chlorine atom, Q represents Q8J, and a combination of structure of G and substituents R^{X7}, R^{X8}, R^{X9} and R^{X10} applied to the structure of G represents any combination described in Combination B (hereinafter, referred to as Compound group SX403).

A compound represented by formula (1M) (hereinafter, referred to as Compound (1M)):

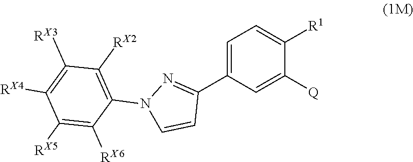

wherein R¹ represents a methyl group, Q represents Q3, R³ represents a methoxy group, and a combination of R^{X2}, R^{X3}, R^{X4}, R^{X5} and R^{X6} represents any combination described in Combination A (hereinafter, referred to as Compound group SX404).

A compound according to Compound (1M) wherein R¹ represents a chlorine atom, Q represents Q3, R³ represents a methoxy group, and a combination of R^{X2}, R^{X3}, R^{X4}, R^{X5} and R^{X6} represents any combination described in Combination A (hereinafter, referred to as Compound group SX405).

A compound according to Compound (1M) wherein R¹ represents a methyl group, Q represents Q4, and a combination of R^{X2}, R^{X3}, R^{X4}, R^{X5} and R^{X6} represents any combination described in Combination A (hereinafter, referred to as Compound group SX406).

A compound according to Compound (1M) wherein R¹ represents a chlorine atom, Q represents Q4, and a combination of R^{X2}, R^{X3}, R^{X4}, R^{X5} and R^{X6} represents any combination described in Combination A (hereinafter, referred to as Compound group SX407).

A compound according to Compound (1M) wherein R¹ represents a methyl group, Q represents Q5, and a combination of R^{X2}, R^{X3}, R^{X4}, R^{X5} and R^{X6} represents any combination described in Combination A (hereinafter, referred to as Compound group SX408).

A compound according to Compound (1M) wherein R¹ represents a chlorine atom, Q represents Q5, and a combination of R^{X2}, R^{X3}, R^{X4}, R^{X5} and R^{X6} represents any combination described in Combination A (hereinafter, referred to as Compound group SX409).

A compound according to Compound (1M) wherein R¹ represents a methyl group, Q represents Q6, and a combination of R^{X2}, R^{X3}, R^{X4}, R^{X5} and R^{X6} represents any combination described in Combination A (hereinafter, referred to as Compound group SX410).

A compound according to Compound (1M) wherein R¹ represents a chlorine atom, Q represents Q6, and a combination of R^{X2}, R^{X3}, R^{X4}, R^{X5} and R^{X6} represents any combination described in Combination A (hereinafter, referred to as Compound group SX411).

A compound according to Compound (1M) wherein R¹ represents a methyl group, Q represents Q7, L² represents an oxygen atom, and a combination of R^{X2}, R^{X3}, R^{X4}, R^{X5} and R^{X6} represents any combination described in Combination A (hereinafter, referred to as Compound group SX412).

A compound according to Compound (1M) wherein R¹ represents a chlorine atom, Q represents Q7, L² represents an oxygen atom, and a combination of R^{X2}, R^{X3}, R^{X4}, R^{X5} and R^{X6} represents any combination described in Combination A (hereinafter, referred to as Compound group SX413).

A compound according to Compound (1M) wherein R¹ represents a methyl group, Q represents Q7, L² represents NH, and a combination of R^{X2}, R^{X3}, R^{X4}, R^{X5} and R^{X6} represents any combination described in Combination A (hereinafter, referred to as Compound group SX414).

A compound according to Compound (1M) wherein R¹ represents a chlorine atom, Q represents Q7, L² represents NH, and a combination of R^{X2}, R^{X3}, R^{X4}, R^{X5} and R^{X6} represents any combination described in Combination A (hereinafter, referred to as Compound group SX415).

A compound according to Compound (1M) wherein R¹ represents a methyl group, Q represents Q8A, and a combination of R^{X2}, R^{X3}, R^{X4}, R^{X5} and R^{X6} represents any combination described in Combination A (hereinafter, referred to as Compound group SX416).

A compound according to Compound (1M) wherein R¹ represents a chlorine atom, Q represents Q8A, and a combination of R^{X2}, R^{X3}, R^{X4}, R^{X5} and R^{X6} represents any combination described in Combination A (hereinafter, referred to as Compound group SX417).

A compound according to Compound (1M) wherein R¹ represents a methyl group, Q represents Q8D, and a combination of R^{X2}, R^{X3}, R^{X4}, R^{X5} and R^{X6} represents any combination described in Combination A (hereinafter, referred to as Compound group SX418).

A compound according to Compound (1M) wherein R¹ represents a chlorine atom, Q represents Q8D, and a combination of R^{X2}, R^{X3}, R^{X4}, R^{X5} and R^{X6} represents any combination described in Combination A (hereinafter, referred to as Compound group SX419).

A compound according to Compound (1M) wherein R¹ represents a methyl group, Q represents Q8J, and a combination of R^{X2}, R^{X3}, R^{X4}, R^{X5} and R^{X6} represents any combination described in Combination A (hereinafter, referred to as Compound group SX420).

A compound according to Compound (1M) wherein R¹ represents a chlorine atom, Q represents Q8J, and a combination of R^{X2}, R^{X3}, R^{X4}, R^{X5} and R^{X6} represents any combination described in Combination A (hereinafter, referred to as Compound group SX421).

A compound represented by formula (1N) (hereinafter, referred to as Compound (1N)):

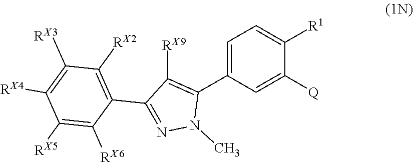

wherein R¹ represents a methyl group, Q represents Q3, R³ represents a methoxy group, R^{X9} represents a hydrogen atom, and a combination of R^{X2}, R^{X3}, R^{X4}, R^{X5} and R^{X6} represents any combination described in Combination A (hereinafter, referred to as Compound group SX422)).

A compound according to Compound (1N) wherein R¹ represents a hydrogen atom, Q represents Q3, R³ represents a methoxy group, R^{X9} represents a methyl group, and a combination of R^{X2}, R^{X3}, R^{X4}, R^{X5} and R^{X6} represents any combination described in Combination A (hereinafter, referred to as Compound group SX423).

A compound according to Compound (1N) wherein R¹ represents a chlorine atom, Q represents Q3, R³ represents a methoxy group, R^{X9} represents a hydrogen atom, and a combination of R^{X2}, R^{X3}, R^{X4}, R^{X5} and R^{X6} represents any combination described in Combination A (hereinafter, referred to as Compound group SX424).

A compound according to Compound (1N) wherein R¹ represents a chlorine atom, Q represents Q3, R³ represents a methoxy group, R^{X9} represents a methyl group, and a combination of R^{X2}, R^{X3}, R^{X4}, R^{X5} and R^{X6} represents any combination described in Combination A (hereinafter, referred to as Compound group SX425).

A compound according to Compound (1N) wherein R¹ represents a methyl group, Q represents Q4, R^{X9} represents a hydrogen atom, and a combination of R^{X2}, R^{X3}, R^{X4}, R^{X5} and R^{X6} represents any combination described in Combination A (hereinafter, referred to as Compound group SX426).

A compound according to Compound (1N) wherein R¹ represents a hydrogen atom, Q represents Q4, R^{X9} represents a methyl group, and a combination of R^{X2}, R^{X3}, R^{X4}, R^{X5} and R^{X6} represents any combination described in Combination A (hereinafter, referred to as Compound group SX427).

A compound according to Compound (1N) wherein R¹ represents a chlorine atom, Q represents Q4, R^{X9} represents a hydrogen atom, and a combination of R^{X2}, R^{X3}, R^{X4}, R^{X5} and R^{X6} represents any combination described in Combination A (hereinafter, referred to as Compound group SX428).

A compound according to Compound (1N) wherein R¹ represents a chlorine atom, Q represents Q4, R^{X9} represents a methyl group, and a combination of R^{X2}, R^{X3}, R^{X4}, R^{X5} and R^{X6} represents any combination described in Combination A (hereinafter, referred to as Compound group SX429).

A compound according to Compound (1N) wherein R¹ represents a methyl group, Q represents Q5, R^{X9} represents a hydrogen atom, and a combination of R^{X2}, R^{X3}, R^{X4}, R^{X5} and R^{X6} represents any combination described in Combination A (hereinafter, referred to as Compound group SX430).

A compound according to Compound (1N) wherein R¹ represents a hydrogen atom, Q represents Q5, R^{X9} represents a methyl group, and a combination of R^{X2}, R^{X3}, R^{X4}, R^{X5} and R^{X6} represents any combination described in Combination A (hereinafter, referred to as Compound group SX431).

A compound according to Compound (1N) wherein R¹ represents a chlorine atom, Q represents Q5, R^{X9} represents a hydrogen atom, and a combination of R^{X2}, R^{X3}, R^{X4}, R^{X5} and R^{X6} represents any combination described in Combination A (hereinafter, referred to as Compound group SX432).

A compound according to Compound (1N) wherein R¹ represents a chlorine atom, Q represents Q5, R^{X9} represents a methyl group, and a combination of R^{X2}, R^{X3}, R^{X4}, R^{X5} and R^{X6} represents any combination described in Combination A (hereinafter, referred to as Compound group SX433).

A compound according to Compound (1N) wherein R¹ represents a methyl group, Q represents Q6, R^{X9} represents a hydrogen atom, and a combination of R^{X2}, R^{X3}, R^{X4}, R^{X5} and R^{X6} represents any combination described in Combination A (hereinafter, referred to as Compound group SX434).

A compound according to Compound (1N) wherein R¹ represents a hydrogen atom, Q represents Q6, R^{X9} represents a methyl group, and a combination of R^{X2}, R^{X3}, R^{X4}, R^{X5} and R^{X6} represents any combination described in Combination A (hereinafter, referred to as Compound group SX435).

A compound according to Compound (1N) wherein R¹ represents a chlorine atom, Q represents Q6, R^{X9} represents a hydrogen atom, and a combination of R^{X2}, R^{X3}, R^{X4}, R^{X5} and R^{X6} represents any combination described in Combination A (hereinafter, referred to as Compound group SX436).

A compound according to Compound (1N) wherein R¹ represents a chlorine atom, Q represents Q6, R^{X9} represents a methyl group, and a combination of R^{X2}, R^{X3}, R^{X4}, R^{X5} and R^{X6} represents any combination described in Combination A (hereinafter, referred to as Compound group SX437).

A compound according to Compound (1N) wherein R¹ represents a methyl group, Q represents Q7, L² represents an oxygen atom, R^{X9} represents a hydrogen atom, and a combination of R^{X2}, R^{X3}, R^{X4}, R^{X5} and R^{X6} represents any combination described in Combination A (hereinafter, referred to as Compound group SX438).

A compound according to Compound (1N) wherein R¹ represents a hydrogen atom, Q represents Q7, L² represents an oxygen atom, R^{X9} represents a methyl group, and a combination of R^{X2}, R^{X3}, R^{X4}, R^{X5} and R^{X6} represents any combination described in Combination A (hereinafter, referred to as Compound group SX439).

A compound according to Compound (1N) wherein R¹ represents a chlorine atom, Q represents Q7, L² represents an oxygen atom, R^{X9} represents a hydrogen atom, and a combination of R^{X2}, R^{X3}, R^{X4}, R^{X5} and R^{X6} represents any combination described in Combination A (hereinafter, referred to as Compound group SX440).

A compound according to Compound (1N) wherein R¹ represents a chlorine atom, Q represents Q7, L² represents an oxygen atom, R^{X9} represents a methyl group, and a combination of R^{X2}, R^{X3}, R^{X4}, R^{X5} and R^{X6} represents any combination described in Combination A (hereinafter, referred to as Compound group SX441).

A compound according to Compound (1N) wherein R¹ represents a methyl group, Q represents Q7, L² represents NH, R^{X9} represents a hydrogen atom, and a combination of R^{X2}, R^{X3}, R^{X4}, R^{X5} and R^{X6} represents any combination described in Combination A (hereinafter, referred to as Compound group SX442).

A compound according to Compound (1N) wherein R¹ represents a hydrogen atom, Q represents Q7, L² represents NH, R^{X9} represents a methyl group, and a combination of R^{X2}, R^{X3}, R^{X4}, R^{X5} and R^{X6} represents any combination described in Combination A (hereinafter, referred to as Compound group SX443).

A compound according to Compound (1N) wherein R¹ represents a chlorine atom, Q represents Q7, L² represents NH, R^{X9} represents a hydrogen atom, and a combination of R^{X2}, R^{X3}, R^{X4}, R^{X5} and R^{X6} represents any combination described in Combination A (hereinafter, referred to as Compound group SX444).

A compound according to Compound (1N) wherein R¹ represents a chlorine atom, Q represents Q7, L² represents NH, R^{X9} represents a methyl group, and a combination of R^{X2}, R^{X3}, R^{X4}, R^{X5} and R^{X6} represents any combination described in Combination A (hereinafter, referred to as Compound group SX445).

A compound according to Compound (1N) wherein R¹ represents a methyl group, Q represents Q8A, R^{X9} represents a hydrogen atom, and a combination of R^{X2}, R^{X3}, R^{X4}, R^{X5} and R^{X6} represents any combination described in Combination A (hereinafter, referred to as Compound group SX446).

A compound according to Compound (1N) wherein R¹ represents a hydrogen atom, Q represents Q8A, R^{X9} represents a methyl group, and a combination of R^{X2}, R^{X3}, R^{X4}, R^{X5} and R^{X6} represents any combination described in Combination A (hereinafter, referred to as Compound group SX447).

A compound according to Compound (1N) wherein R¹ represents a chlorine atom, Q represents Q8A, R^{X9} represents a hydrogen atom, and a combination of R^{X2}, R^{X3}, R^{X4}, R^{X5} and R^{X6} represents any combination described in Combination A (hereinafter, referred to as Compound group SX448).

A compound according to Compound (1N) wherein R¹ represents a chlorine atom, Q represents Q8A, R^{X9} represents a methyl group, and a combination of R^{X2}, R^{X3}, R^{X4}, R^{X5} and R^{X6} represents any combination described in Combination A (hereinafter, referred to as Compound group SX449).

A compound according to Compound (1N) wherein R¹ represents a methyl group, Q represents Q8D, R^{X9} represents a hydrogen atom, and a combination of R^{X2}, R^{X3}, R^{X4}, R^{X5} and R^{X6} represents any combination described in Combination A (hereinafter, referred to as Compound group SX450).

A compound according to Compound (1N) wherein R¹ represents a hydrogen atom, Q represents Q8D, R^{X9} represents a methyl group, and a combination of R^{X2}, R^{X3}, R^{X4}, R^{X5} and R^{X6} represents any combination described in Combination A (hereinafter, referred to as Compound group SX451).

A compound according to Compound (1N) wherein R¹ represents a chlorine atom, Q represents Q8D, R^{X9} represents a hydrogen atom, and a combination of R^{X2}, R^{X3}, R^{X4}, R^{X5} and R^{X6} represents any combination described in Combination A (hereinafter, referred to as Compound group SX452).

A compound according to Compound (1N) wherein R¹ represents a chlorine atom, Q represents Q8D, R^{X9} represents a methyl group, and a combination of R^{X2}, R^{X3}, R^{X4}, R^{X5} and R^{X6} represents any combination described in Combination A (hereinafter, referred to as Compound group SX453).

A compound according to Compound (1N) wherein R¹ represents a methyl group, Q represents Q8J, R^{X9} represents a hydrogen atom, and a combination of R^{X2}, R^{X3}, R^{X4}, R^{X5} and R^{X6} represents any combination described in Combination A (hereinafter, referred to as Compound group SX454).

A compound according to Compound (1N) wherein R¹ represents a hydrogen atom, Q represents Q8J, R^{X9} represents a methyl group, and a combination of R^{X2}, R^{X3}, R^{X4}, R^{X5} and R^{X6} represents any combination described in Combination A (hereinafter, referred to as Compound group SX455).

A compound according to Compound (1N) wherein R¹ represents a chlorine atom, Q represents Q8J, R^{X9} represents a hydrogen atom, and a combination of R^{X2}, R^{X3}, R^{X4}, R^{X5} and R^{X6} represents any combination described in Combination A (hereinafter, referred to as Compound group SX456).

A compound according to Compound (1N) wherein R¹ represents a chlorine atom, Q represents Q8J, R^{X9} represents a methyl group, and a combination of R^{X2}, R^{X3}, R^{X4}, R^{X5} and R^{X6} represents any combination described in Combination A (hereinafter, referred to as Compound group SX457).

A compound represented by formula (1P) (hereinafter, referred to as Compound (1P)):

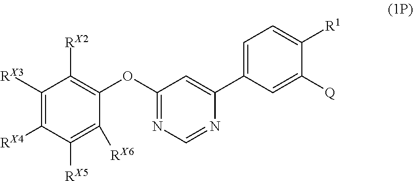

wherein R¹ represents a methyl group, Q represents Q3, R³ represents a methoxy group, and a combination of R^{X2}, R^{X3}, R^{X4}, R^{X5} and R^{X6} represents any combination described in Combination A (hereinafter, referred to as Compound group SX458).

A compound according to Compound (1P) wherein R¹ represents a chlorine atom, Q represents Q3, R³ represents a methoxy group, and a combination of R^{X2}, R^{X3}, R^{X4}, R^{X5} and R^{X6} represents any combination described in Combination A (hereinafter, referred to as Compound group SX459).

A compound according to Compound (1P) wherein R¹ represents a methyl group, Q represents Q4, and a combination of R^{X2}, R^{X3}, R^{X4}, R^{X5} and R^{X6} represents any combination described in Combination A (hereinafter, referred to as Compound group SX460).

A compound according to Compound (1P) wherein R¹ represents a chlorine atom, Q represents Q4, and a combination of R^{X2}, R^{X3}, R^{X4}, R^{X5} and R^{X6} represents any combination described in Combination A (hereinafter, referred to as Compound group SX461).

A compound according to Compound (1P) wherein R¹ represents a methyl group, Q represents Q5, and a combination of R^{X2}, R^{X3}, R^{X4}, R^{X5} and R^{X6} represents any combination described in Combination A (hereinafter, referred to as Compound group SX462).

A compound according to Compound (1P) wherein R¹ represents a chlorine atom, Q represents Q5, and a combination of R^{X2}, R^{X3}, R^{X4}, R^{X5} and R^{X6} represents any combination described in Combination A (hereinafter, referred to as Compound group SX463).

A compound according to Compound (1P) wherein R¹ represents a methyl group, Q represents Q6, and a combination of R^{X2}, R^{X3}, R^{X4}, R^{X5} and R^{X6} represents any combination described in Combination A (hereinafter, referred to as Compound group SX464).

A compound according to Compound (1P) wherein R¹ represents a chlorine atom, Q represents Q6, and a combination of R^{X2}, R^{X3}, R^{X4}, R^{X5} and R^{X6} represents any combination described in Combination A (hereinafter, referred to as Compound group SX465).

A compound according to Compound (1P) wherein R¹ represents a methyl group, Q represents Q7, L² represents an oxygen atom, and a combination of R^{X2}, R^{X3}, R^{X4}, R^{X5} and R^{X6} represents any combination described in Combination A (hereinafter, referred to as Compound group SX466).

A compound according to Compound (1P) wherein R¹ represents a chlorine atom, Q represents Q7, L² represents an oxygen atom, and a combination of R^{X2}, R^{X3}, R^{X4}, R^{X5} and R^{X6} represents any combination described in Combination A (hereinafter, referred to as Compound group SX467).

A compound according to Compound (1P) wherein R¹ represents a methyl group, Q represents Q7, L² represents NH, and a combination of R^{X2}, R^{X3}, R^{X4}, R^{X5} and R^{X6} represents any combination described in Combination A (hereinafter, referred to as Compound group SX468).

A compound according to Compound (1P) wherein R¹ represents a chlorine atom, Q represents Q7, L² represents NH, and a combination of R^{X2}, R^{X3}, R^{X4}, R^{X5} and R^{X6} represents any combination described in Combination A (hereinafter, referred to as Compound group SX469).

A compound according to Compound (1P) wherein R¹ represents a methyl group, Q represents Q8A, and a combination of R^{X2}, R^{X3}, R^{X4}, R^{X5} and R^{X6} represents any combination described in Combination A (hereinafter, referred to as Compound group SX470).

A compound according to Compound (1P) wherein R¹ represents a chlorine atom, Q represents Q8A, and a combination of R^{X2}, R^{X3}, R^{X4}, R^{X5} and R^{X6} represents any combination described in Combination A (hereinafter, referred to as Compound group SX471).

A compound according to Compound (1P) wherein R¹ represents a methyl group, Q represents Q8D, and a combination of R^{X2}, R^{X3}, R^{X4}, R^{X5} and R^{X6} represents any combination described in Combination A (hereinafter, referred to as Compound group SX472).

A compound according to Compound (1P) wherein R¹ represents a chlorine atom, Q represents Q8D, and a combination of R^{X2}, R^{X3}, R^{X4}, R^{X5} and R^{X6} represents any combination described in Combination A (hereinafter, referred to as Compound group SX473).

A compound according to Compound (1P) wherein R¹ represents a methyl group, Q represents Q8J, and a combination of R^{X2}, R^{X3}, R^{X4}, R^{X5} and R^{X6} represents any combination described in Combination A (hereinafter, referred to as Compound group SX474).

A compound according to Compound (1P) wherein R¹ represents a chlorine atom, Q represents Q8J, and a combination of R^{X2}, R^{X3}, R^{X4}, R^{X5} and R^{X6} represents any combination described in Combination A (hereinafter, referred to as Compound group SX475).

A compound represented by formula (1U) (hereinafter, referred to as Compound (1U)):

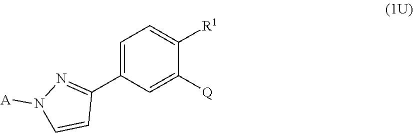

[wherein A represents any one of groups represented by formulae AA1 to AA4

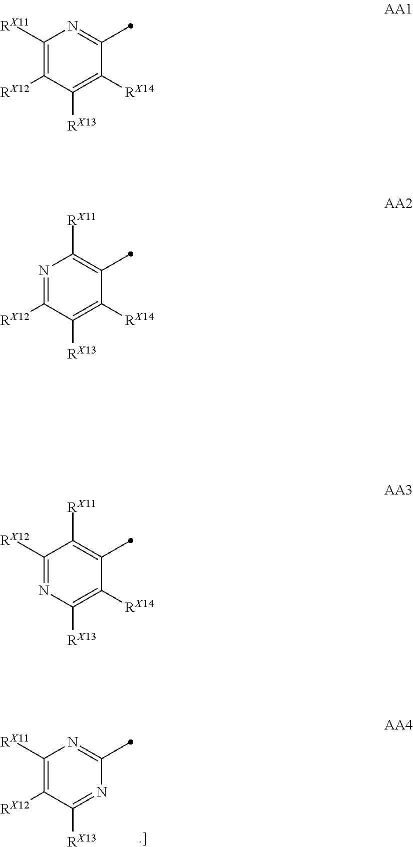

wherein R¹ represents a methyl group, Q represents Q3, R³ represents a methoxy group, and a combination of structure of A and substituents of R^{X11}, R^{X12}, R^{X13} and R^{X14} applied to the structure of A represents any combination described in Combination D (hereinafter, referred to as Compound group SX476).

Combination D consists of substituent numbers ZD1 to ZD334. Substituent numbers ZD1 to ZD334 represent combinations of structure of A and substituents of R^{X11}, R^{X12}. R^{X13} and R^{X14} applied to the structure of A according to Compound (1U), compounds represented by formula (1V), compounds represented by formula (2D), and compounds represented by formula (2E). And hereinafter, they are referred to as [substituent number; A,R^{X11},R^{X12},R^{X13},R^{X14}]. For example, the substituent number ZD2 refers to a combination wherein A represents AA1, R^{X11} represents a methyl group, and each of R^{X12}, R^{X13} and R^{X14} represents a hydrogen atom.
Combination D: [ZD1;AA1,H,H,H,H], [ZD2;AA1,Me,H,H,H], [ZD3;AA1,Et,H,H,H], [ZD 4;AA1,Pr,H,H,H], [ZD5;AA1,i-Pr,H,H,H], [ZD6;AA1,c-Pr,H,H,H], [ZD7;AA1,Ph,H,H,H], [ZD8;AA1,OMe,H,H,H], [ZD9;AA1,O Et,H,H,H], [ZD10;AA1,OPh,H,H,H], [ZD11;AA1,CF₃,H,H,H], [ZD12;A A1,F,H,H,H], [ZD13;AA1,Cl,H,H,H], [ZD14;AA1,Br,H,H,H], [ZD15;A A1,CN,H,H,H], [ZD16;AA1,H,Me,H,H], [ZD17;AA1,H,Et,H,H], [ZD18; AA1,H,Pr,H,H], [ZD19;AA1,H,i-Pr,H,H], [ZD20;AA1,H,c-Pr,H,H], [ZD21;AA1,H,Ph,H,H], [ZD22;AA1,H,OMe,H,H], [ZD23;AA1, H,OEt,H,H], [ZD24;AA1,H,OPh,H,H], [ZD25;AA1,H,CF₃,H,H], [ZD26; AA1,H,F,H,H], [ZD27;AA1,H,Cl,H,H], [ZD28;AA1,H,Br,H,H], [ZD29; AA1,H,CN,H,H], [ZD30;AA1,H,H,Me,H], [ZD31;AA1,H,H,Et,H], [ZD32;AA1,H,H,Pr,H], [ZD33;AA1,H,H,i-Pr,H], [ZD34;AA1,H,H,c-Pr,H], [ZD35;AA1,H,H,Ph,H], [ZD36;AA1,H,H,OMe,H], [ZD37;AA1,H, H,OEt,H], [ZD38;AA1,H,H,OPh,H], [ZD39;AA1,H,H,CF₃,H], [ZD40;AA 1,H,H,F,H], [ZD41;AA1,H,H,Cl,H], [ZD42;AA1,H,H.Br,H], [ZD43;AA 1,H,H,CN,H], [ZD44;AA1,H,H,H,Me], [ZD45;AA1,H,H,H,Et], [ZD46;A A1,H,H,H,Pr], [ZD47;AA1,H,H,H,i-Pr], [ZD48;AA1,H,H,H,c-Pr], [ZD49;AA1,H,H,H,Ph], [ZD50;AA1,H,H,H,OMe], [ZD51;AA1,H,H, H,OEt], [ZD52;AA1,H,H,H,OPh], [ZD53;AA1,H,H,H,CF₃], [ZD54;AA1, H,H,H,F], [ZD55;AA1,H,H,H,Cl], [ZD56;AA1,H,H,H,Br], [ZD57;AA1, H,H,H,CN], [ZD58;AA1,Me,Me,H,H], [ZD59;AA1,Me,Et,H,H], [ZD60;A A1,Me,OMe,H,H], [ZD61;AA1,Me,OEt,H,H], [ZD62;AA1,Me,SMe,H,H], [ZD63;AA1,Me,F,H,H], [ZD64;AA1,Me,Cl,H,H], [ZD65;AA1,Me,CN,H, H], [ZD66;AA1,F,Me,H,H], [ZD67;AA1,F,Et,H,H], [ZD68;AA1,F,OMe, H,H], [ZD69;AA1,F,OEt,H,H], [ZD70;AA1,F,SMe,H,H], [ZD71;AA1,F, F,H,H], [ZD72;AA1,F,Cl,H,H], [ZD73;AA1,F,CN,H,H], [ZD74;AA1,C1,Me,H,H], [ZD75;AA1,Cl,Et,H,H], [ZD76;AA1,Cl,OMe,H,H], [ZD77;A A1,Cl,OEt,H,H], [ZD78;AA1,Cl,SMe,H,H], [ZD79;AA1,Cl,F,H,H], [Z D80;AA1,Cl,Cl,H,H], [ZD81;AA1,Cl,CN,H,H], [ZD82;AA1,OMe,Me,H, H], [ZD83;AA1,OMe,Et,H,H], [ZD84;AA1,OMe,OMe,H,H], [ZD85;AA1,O Me,OEt,H,H], [ZD86;AA1,OMe,SMe,H,H], [ZD87;AA1,OMe,F,H,H], [ZD 88;AA1,OMe,Cl,H,H], [ZD89;AA1,OMe,CN,H,H], [ZD90;AA1,SMe,Me,H,H], [ZD91;AA1,SMe,Et,H,H], [ZD92;AA1,SMe,OMe,H,H], [ZD93;AA1, SMe,OEt,H,H], [ZD94;AA1,SMe,SMe,H,H], [ZD95;AA1,SMe,F,H,H], [Z D96;AA1,SMe,Cl,H,H], [ZD97;AA1,SMe,CN,H,H], [ZD98;AA2,H,H,H,H], [ZD99;AA2,Me,H,H,H], [ZD100;AA2,Et,H,H,H], [ZD101;AA2,Pr,H, H,H], [ZD102;AA2,i-Pr,H,H,H], [ZD103;AA2,c-Pr,H,H,H], [ZD104;AA2,Ph,H,H,H], [ZD105;AA2,OMe,H,H,H], [ZD106;AA2,OEt,H,H,H], [ZD107;AA2,OPh,H,H,H], [ZD108;AA2,CF₃,H,H,H], [ZD109;AA2,F,H,H,H], [ZD110;AA2,Cl,H,H,H], [ZD111;AA2,Br,H,H,H], [ZD112;AA2,CN,H,H,H], [ZD113;AA2,H,Me,H,H], [ZD114;AA2,H, Et,H,H], [ZD115;AA2,H,Pr,H,H], [ZD116;AA2,H,i-Pr,H,H], [ZD117;AA2,H,c-Pr,H,H], [ZD118;AA2,H,Ph,H,H], [ZD119;AA2,H,OMe,H,H], [ZD120;A A2,H,OEt,H,H], [ZD121;AA2,H,OPh,H,H], [ZD122;AA2,H,CF₃,H,H], [ZD123;AA2,H,F,H,H], [ZD124;AA2,H,Cl,H,H], [ZD125;AA2,H,Br,H,H], [ZD126;AA2,H,CN,H,H], [ZD127;AA2,H,H,Me,H], [ZD128;AA2,H,H, Et,H], [ZD129;AA2,H,H,Pr,H], [ZD130;AA2,H,H,i-Pr,H], [ZD131;AA2,H,H,c-Pr,H], [ZD132;AA2,H,H,Ph,H], [ZD133;AA2,H,H,OMe,H], [ZD134;AA2,H,H,OEt,H], [ZD135;AA2,H,H,OPh,H], [ZD136;AA2,H,H,CF₃,H], [ZD 137;AA2,H,H,F,H], [ZD138;AA2,H,H,Cl,H], [ZD139;AA2,H,H,Br,H], [ZD140;AA2,H,H,CN,H], [ZD141;AA2,H,H,H,Me], [ZD142;AA2,H,H,H, Et], [ZD143;AA2,H,H,H,Pr], [ZD144;AA2,H,H,H,i-Pr], [ZD145;AA2,H,H,H,c-Pr], [ZD146;AA2,H,H,H,Ph], [ZD147;AA2,H,H,H,OMe], [ZD148;AA2,H,H,H,OEt], [ZD149;AA2,H,H,H,OPh], [ZD150;AA2,H,H,H,CF₃], [ZD15 1;AA2,H,H,H,F], [ZD152;AA2,H,H,H,Cl], [ZD153;AA2,H,H,H,Br], [Z D154;AA2,H,H,H,CN], [ZD155;AA2,Me,Me,H,H], [ZD156;AA2,Me,Et,H,H], [ZD157;AA2,Me,OMe,H,H], [ZD158;AA2,Me,OEt,H,H], [ZD159;AA 2,Me,SMe,H,H], [ZD160;AA2,Me,F,H,H], [ZD161;AA2,Me,Cl,H,H], [Z D162;AA2,Me,CN,H,H], [ZD163;AA2,F,Me,H,H], [ZD164;AA2,F,Et,H, H], [ZD165;AA2,F,OMe,H,H], [ZD166;AA2,F,OEt,H,H], [ZD167;AA2,F,SMe,H,H], [ZD168;AA2,F,F,H,H], [ZD169;AA2,F,Cl,H,H], [ZD170;A A2,F,CN,H,H], [ZD171;AA2,Cl,Me,H,H], [ZD172;AA2,Cl,Et,H,H], [Z D173;AA2,Cl,OMe,H,H], [ZD174;AA2,Cl,OEt,H,H], [ZD175;AA2,Cl,S Me,H,H], [ZD176;AA2,Cl,F,H,H], [ZD177;AA2,Cl,Cl,H,H], [ZD178;A A2,Cl,CN,H,H], [ZD179;AA2,OMe,Me,H,H], [ZD180;AA2,OMe,Et,H,H], [ZD181;AA2,OMe,OMe,H,H], [ZD182;AA2,OMe,OEt,H,H], [ZD183;AA2,OMe,SMe,H,H], [ZD184;AA2,OMe,F,H,H], [ZD185;AA2,OMe,Cl,H,H], [ZD186;AA2,OMe,CN,H,H], [ZD187;AA2,SMe,Me,H,H], [ZD188;AA2,SM e,Et,H,H], [ZD189;AA2,SMe,OMe,H,H], [ZD190;AA2,SMe,OEt,H,H], [ZD191;AA2,SMe,SMe,H,H], [ZD192;AA2,SMe,F,H,H], [ZD193;AA2,SMe,Cl,H,H], [ZD194;AA2,SMe,CN,H,H], [ZD195;AA3,H,H,H,H], [ZD196; AA3,Me,H,H,H], [ZD197;AA3,Et,H,H,H], [ZD198;AA3,Pr,H,H,H], [ZD 199;AA3,i-Pr,H,H,H], [ZD200;AA3,c-Pr,H,H,H], [ZD201;AA3,Ph,H,H,H], [ZD202;AA3,OMe,H,H,H], [ZD203;AA3,OEt,H,H,H], [ZD204;AA3,OPh,H,H,H], [ZD205;AA3,CF₃,H,H,H], [ZD206;AA3,F,H,H,H], [ZD207;AA3,Cl,H,H,H], [ZD208;AA3,Br,H,H,H], [ZD209;AA3,CN,H,H,H], [ZD210;AA3,H,Me,H,H], [ZD211;AA3,H, Et,H,H], [ZD212;AA3,H,Pr,H,H], [ZD213;AA3,H,i-Pr,H,H], [ZD214;AA3,H,c-Pr,H,H], [ZD215;AA3,H,Ph,H,H], [ZD216;AA3,H,OMe,H,H], [ZD217;A A3, H, OEt, H, H], [ZD218;AA3, H, OPh, H, H], [ZD219;AA3, H, CF₃, H, H], [ZD220;AA3,H,F,H,H], [ZD221;AA3,H,Cl,H,H], [ZD222;AA3,H,Br,H,H], [ZD223;AA3,H,CN,H,H], [ZD224;AA3,H,H,Me,H], [ZD225;AA3,H,H, Et,H], [ZD226;AA3,H,H,Pr,H], [ZD227;AA3,H,H,i-Pr,H], [ZD228;AA3,H,H,c-Pr,H], [ZD229;AA3,H,H,Ph,H], [ZD230;AA3,H,H,OMe,H], [ZD231;AA3,H,H,OEt,H], [ZD232;AA3,H,H,OPh,H], [ZD233;AA3,H,H,CF₃,H], [ZD 234;AA3,H,H,F,H], [ZD235;AA3,H,H,Cl,H], [ZD236;AA3,H,H,Br,H], [ZD237;AA3,H,H,CN,H], [ZD238;AA3,H,H,H,Me], [ZD239;AA3,H,H,H, Et], [ZD240;AA3,H,H,H,Pr], [ZD241;AA3,H,H,H,i-Pr], [ZD242;AA3,H,H,H,c-Pr], [ZD243;AA3,H,H,H,Ph], [ZD244;AA3,H,H,H,OMe], [ZD245;AA3,H,H,H,OEt], [ZD246;AA3,H,H,H,OPh], [ZD247;AA3,H,H,H,CF₃], [ZD24 8;AA3,H,H,H,F], [ZD249;AA3,H,H,H,Cl], [ZD250;AA3,H,H,H,Br], [Z D251;AA3,H,H,H,CN], [ZD252;AA3,Me,Me,H,H], [ZD253;AA3,Me,Et,H,H], [ZD254;AA3,Me,OMe,H,H], [ZD255;AA3,Me,OEt,H,H], [ZD256;AA 3,Me,SMe,H,H], [ZD257;AA3,Me,F,H,H], [ZD258;AA3,Me,Cl,H,H], [Z D259;AA3,Me,CN,H,H], [ZD260;AA3,F,Me,H,H], [ZD261;AA3,F,Et,H, H], [ZD262;AA3,F,OMe,H,H], [ZD263;AA3,F,OEt,H,H], [ZD264;AA3,F,SMe,H,H], [ZD265;AA3,F,F,H,H], [ZD266;AA3,F,Cl,H,H], [ZD267;A A3,F,CN,H,H], [ZD268;AA3,Cl,Me,H,H], [ZD269;AA3,Cl,Et,H,H], [Z D270;AA3,Cl,OMe,H,H], [ZD271;AA3,Cl,OEt,H,H], [ZD272;AA3,Cl,S Me,H,H], [ZD273;AA3,Cl,F,H,H], [ZD274;AA3,Cl,Cl,H,H], [ZD275;A A3,Cl,CN,H,H], [ZD276;AA3,OMe,Me,H,H], [ZD277;AA3,OMe,Et,H,H], [ZD278;AA3,OMe,OMe,H,H], [ZD279;AA3,OMe,OEt,H,H], [ZD280;AA3,OMe,SMe,H,H], [ZD281;AA3,OMe,F,H,H], [ZD282;AA3,OMe,Cl,H,H], [ZD283;AA3,OMe,CN,H,H], [ZD284;AA3,SMe,Me,H,H], [ZD285;AA3,SM e,Et,H,H], [ZD286;AA3,SMe,OMe,H,H], [ZD287;AA3,SMe,OEt,H,H], [ZD288;AA3,SMe,SMe,H,H], [ZD289;AA3,SMe,F,H,H], [ZD290;AA3,SMe,Cl,H,H], [ZD291;AA3,SMe,CN,H,H], [ZD292;AA4,H,H,H,-], [ZD293; AA4,Me,H,H,-], [ZD294;AA4,Et,H,H,-], [ZD295;AA4,Pr,H,H,-], [ZD 296;AA4,i-Pr,H,H,-], [ZD297;AA4,c-Pr,H,H,-], [ZD298;AA4, Ph,H,H, -], [ZD299;AA4,OMe,H,H, -], [ZD300;AA4,OEt,H,H, -], [ZD301;AA4,OPh,H,H, -], [ZD302;AA4,CF₃,H,H, -], [ZD303;AA4,F,H,H, -], [ZD304;AA4,Cl,H,H, -], [ZD305;AA4,Br,H,H, -], [ZD306;AA4, CN,H,H, -], [ZD307;AA4,H,Me,H, -], [ZD308;AA4,H, Et,H,-], [ZD309;AA4,H,Pr,H,-], [ZD310;AA4,H,i-Pr,H,-], [ZD311;AA4,H,c-Pr,H,-], [ZD312;AA4,H,Ph,H, -], [ZD313;AA4,H,OMe,H, -], [ZD314;A A4,H,OEt,H, -], [ZD315;AA4,H,OPh,H, -], [ZD316;AA4,H,CF₃,H, -], [ZD317;AA4,H,F,H,-], [ZD318;AA4,H,Cl,H,-], [ZD319;AA4,H,Br,H,-], [ZD320;AA4,H,CN,H,-], [ZD321;AA4,H,H,Me,-], [ZD322;AA4,H,H, Et,-], [ZD323;AA4,H,H,Pr,-], [ZD324;AA4,H,H,i-Pr,-], [ZD325;AA4,H,H,c-Pr,-], [ZD326;AA4,H,H,Ph, -], [ZD327;AA4,H,H,OMe, -], [ZD328;AA4,H,H,OEt,-], [ZD329;AA4,H,H,OPh, -], [ZD330;AA4,H,H,CF₃, -], [ZD 331;AA4,H,H,F,-], [ZD332;AA4,H,H,Cl,-], [ZD333;AA4,H,H,Br,-], [ZD334;AA4,H,H,CN,-]

A compound according to Compound (1U) wherein R¹ represents a chlorine atom, Q represents Q3, R³ represents a methoxy group, and a combination of structure of A and substituents R^{X11}, R^{X12}, R^{X13} and R^{X14} applied to the structure of A represents any combination described in Combination D (hereinafter, referred to as Compound group SX477).

A compound according to Compound (1U) wherein R¹ represents a methyl group, Q represents Q4, and a combination of structure of A and substituents R^{X11}, R^{X12}, R^{X13} and R^{X14} applied to the structure of A represents any combination described in Combination D (hereinafter, referred to as Compound group SX478).

A compound according to Compound (1U) wherein R¹ represents a chlorine atom, Q represents Q4, and a combination of structure of A and substituents R^{X11}, R^{X12}, R^{X13} and R^{X14} applied to the structure of A represents any combination described in Combination D (hereinafter, referred to as Compound group SX479).

A compound according to Compound (1U) wherein R¹ represents a methyl group, Q represents Q5, and a combination of structure of A and substituents R^{X11}, R^{X12}, R^{X13} and R^{X14} applied to the structure of A represents any combination described in Combination D (hereinafter, referred to as Compound group SX480).

A compound according to Compound (1U) wherein R¹ represents a chlorine atom, Q represents Q5, and a combination of structure of A and substituents R^{X11}, R^{X12}, R^{X13} and R^{X14} applied to the structure of A represents any combination described in Combination D (hereinafter, referred to as Compound group SX481).

A compound according to Compound (1U) wherein R¹ represents a methyl group, Q represents Q6, and a combination of structure of A and substituents R^{X11}, R^{X12}, R^{X13} and R^{X14} applied to the structure of A represents any combination described in Combination D (hereinafter, referred to as Compound group SX482).

A compound according to Compound (1U) wherein R¹ represents a chlorine atom, Q represents Q6, and a combination of structure of A and substituents R^{X11}, R^{X12}, R^{X13} and R^{X14} applied to the structure of A represents any combination described in Combination D (hereinafter, referred to as Compound group SX483).

A compound according to Compound (1U) wherein R¹ represents a methyl group, Q represents Q7, L² represents an oxygen atom, and a combination of structure of A and substituents R^{X11}, R^{X12}, R^{X13} and R^{X14} applied to the structure of A represents any combination described in Combination D (hereinafter, referred to as Compound group SX484).

A compound according to Compound (1U) wherein R¹ represents a chlorine atom, Q represents Q7, L² represents an oxygen atom, and a combination of structure of A and substituents R^{X11}, R^{X12}, R^{X13} and R^{X14} applied to the structure of A represents any combination described in Combination D (hereinafter, referred to as Compound group SX485).

A compound according to Compound (1U) wherein R¹ represents a methyl group, Q represents Q7, L² represents NH, and a combination of structure of A and substituents R^{X11}, R^{X12}. R^{X13} and R^{X14} applied to the structure of A represents any combination described in Combination D (hereinafter, referred to as Compound group SX486).

A compound according to Compound (1U) wherein R¹ represents a chlorine atom, Q represents Q7, L² represents NH, and a combination of structure of A and substituents R^{X11}, R^{X12}, R^{X13} and R^{X14} applied to the structure of A represents any combination described in Combination D (hereinafter, referred to as Compound group SX487).

A compound according to Compound (1U) wherein R¹ represents a methyl group, Q represents Q8A, and a combination of structure of A and substituents R^{X11}, R^{X12}, R^{X13} and R^{X14} applied to the structure of A represents any combination described in Combination D (hereinafter, referred to as Compound group SX488).

A compound according to Compound (1U) wherein R¹ represents a chlorine atom, Q represents Q8A, and a combination of structure of A and substituents R^{X11}, R^{X12}, R^{X13} and R^{X14} applied to the structure of A represents any combination described in Combination D (hereinafter, referred to as Compound group SX489).

A compound according to Compound (1U) wherein R¹ represents a methyl group, Q represents Q8D, and a combination of structure of A and substituents R^{X11}, R^{X12}, R^{X13} and R^{X14} applied to the structure of A represents any combination described in Combination D (hereinafter, referred to as Compound group SX490).

A compound according to Compound (1U) wherein R¹ represents a chlorine atom, Q represents Q8D, and a combination of structure of A and substituents R^{X11}, R^{X12}, R^{X13} and R^{X14} applied to the structure of A represents any combination described in Combination D (hereinafter, referred to as Compound group SX491).

A compound according to Compound (1U) wherein R¹ represents a methyl group, Q represents Q8J, and a combination of structure of A and substituents R^{X11}, R^{X12}, R^{X13} and R^{X14} applied to the structure of A represents any combination described in Combination D (hereinafter, referred to as Compound group SX492).

A compound according to Compound (1U) wherein R¹ represents a chlorine atom, Q represents Q8J, and a combination of structure of A and substituents R^{X11}, R^{X12}, R^{X13} and R^{X14} applied to the structure of A represents any combination described in Combination D (hereinafter, referred to as Compound group SX493).

A compound represented by formula (1V) (hereinafter, referred to as Compound (1V)):

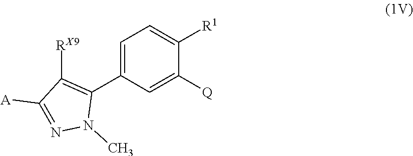

[wherein A represents any one of groups represented by formulae AA1 to AA4.]
wherein R¹ represents a methyl group, Q represents Q3, R³ represents a methoxy group, R^{X9} represents a hydrogen atom, and a combination of structure of A and substituents R^{X11}, R^{X12}, R^{X13} and R^{X14} applied to the structure of A represents any combination described in Combination D (hereinafter, referred to as Compound group SX494).

A compound according to Compound (1V) wherein R¹ represents a methyl group, Q represents Q3, R³ represents a methoxy group, R^{X9} represents a methyl group, and a combination of structure of A and substituents R^{X11}, R^{X12}, R^{X13} and R^{X14} applied to the structure of A represents any combination described in Combination D (hereinafter, referred to as Compound group SX495).

A compound according to Compound (1V) wherein R¹ represents a chlorine atom, Q represents Q3, R³ represents a methoxy group, R^{X9} represents a hydrogen atom, and a combination of structure of A and substituents R^{X11}, R^{X12}, R^{X13} and R^{X14} applied to the structure of A represents any combination described in Combination D (hereinafter, referred to as Compound group SX496).

A compound according to Compound (1V) wherein R¹ represents a chlorine atom, Q represents Q3, R³ represents a methoxy group, R^{X9} represents a methyl group, and a combination of structure of A and substituents R^{X11}, R^{X12}, R^{X13} and R^{X14} applied to the structure of A represents any combination described in Combination D (hereinafter, referred to as Compound group SX497).

A compound according to Compound (1V) wherein R¹ represents a methyl group, Q represents Q4, R^{X9} represents a hydrogen atom, and a combination of structure of A and substituents R^{X11}, R^{X12}, R^{X13} and R^{X14} applied to the structure of A represents any combination described in Combination D (hereinafter, referred to as Compound group SX498).

A compound according to Compound (1V) wherein R¹ represents a methyl group, Q represents Q4, R^{X9} represents a methyl group, and a combination of structure of A and substituents R^{X11}, R^{X12}, R^{X13} and R^{X14} applied to the structure of A represents any combination described in Combination D (hereinafter, referred to as Compound group SX499).

A compound according to Compound (1V) wherein R¹ represents a chlorine atom, Q represents Q4, R^{X9} represents a hydrogen atom, and a combination of structure of A and substituents R^{X11}, R^{X12}, R^{X13} and R^{X14} applied to the structure of A represents any combination described in Combination D (hereinafter, referred to as Compound group SX500).

A compound according to Compound (1V) wherein R¹ represents a chlorine atom, Q represents Q4, R^{X9} represents a methyl group, and a combination of structure of A and substituents R^{X11}, R^{X12}, R^{X13} and R^{X14} applied to the structure of A represents any combination described in Combination D (hereinafter, referred to as Compound group SX501).

A compound according to Compound (1V) wherein R¹ represents a methyl group, Q represents Q5, R^{X9} represents a hydrogen atom, and a combination of structure of A and substituents R^{X11}, R^{X12}, R^{X13} and R^{X14} applied to the structure of A represents any combination described in Combination D (hereinafter, referred to as Compound group SX502).

A compound according to Compound (1V) wherein R¹ represents a methyl group, Q represents Q5, R^{X9} represents a methyl group, and a combination of structure of A and substituents R^{X11}, R^{X12}, R^{X13} and R^{X14} applied to the structure of A represents any combination described in Combination D (hereinafter, referred to as Compound group SX503).

A compound according to Compound (1V) wherein R¹ represents a chlorine atom, Q represents Q5, R^{X9} represents a hydrogen atom, and a combination of structure of A and substituents R^{X11}, R^{X12}, R^{X13} and R^{X14} applied to the structure of A represents any combination described in Combination D (hereinafter, referred to as Compound group SX504).

A compound according to Compound (1V) wherein R¹ represents a chlorine atom, Q represents Q5, R^{X9} represents a methyl group, and a combination of structure of A and substituents R^{X11}, R^{X12}, R^{X13} and R^{X14} applied to the structure of A represents any combination described in Combination D (hereinafter, referred to as Compound group SX505).

A compound according to Compound (1V) wherein R¹ represents a methyl group, Q represents Q6, R^{X9} represents a hydrogen atom, and a combination of structure of A and substituents R^{X11}, R^{X12}, R^{X13} and R^{X14} applied to the structure of A represents any combination described in Combination D (hereinafter, referred to as Compound group SX506).

A compound according to Compound (1V) wherein R¹ represents a methyl group, Q represents Q6, R^{X9} represents a methyl group, and a combination of structure of A and substituents R^{X11}, R^{X12}, R^{X13} and R^{X14} applied to the structure of A represents any combination described in Combination D (hereinafter, referred to as Compound group SX507).

A compound according to Compound (1V) wherein R¹ represents a chlorine atom, Q represents Q6, R^{X9} represents a hydrogen atom, and a combination of structure of A and substituents R^{X11}, R^{X12}, R^{X13} and R^{X14} applied to the structure of A represents any combination described in Combination D (hereinafter, referred to as Compound group SX508).

A compound according to Compound (1V) wherein R¹ represents a chlorine atom, Q represents Q6, R^{X9} represents a methyl group, and a combination of structure of A and substituents R^{X11}, R^{X12}, R^{X13} and R^{X14} applied to the structure of A represents any combination described in Combination D (hereinafter, referred to as Compound group SX509).

A compound according to Compound (1V) wherein R¹ represents a methyl group, Q represents Q7, L² represents an oxygen atom, R^{X9} represents a hydrogen atom, and a combination of structure of A and substituents R^{X11}, R^{X12}, R^{X13} and R^{X14} applied to the structure of A represents any combination described in Combination D (hereinafter, referred to as Compound group SX510).

A compound according to Compound (1V) wherein R¹ represents a methyl group, Q represents Q7, L² represents an oxygen atom, R^{X9} represents a methyl group, and a combination of structure of A and substituents R^{X11}, R^{X12}, R^{X13} and R^{X14} applied to the structure of A represents any combination described in Combination D (hereinafter, referred to as Compound group SX511).

A compound according to Compound (1V) wherein R¹ represents a chlorine atom, Q represents Q7, L² represents an oxygen atom, R^{X9} represents a hydrogen atom, and a combination of structure of A and substituents R^{X11}, R^{X12}, R^{X13} and R^{X14} applied to the structure of A represents any combination described in Combination D (hereinafter, referred to as Compound group SX512).

A compound according to Compound (1V) wherein R¹ represents a chlorine atom, Q represents Q7, L² represents an oxygen atom, R^{X9} represents a methyl group, and a combination of structure of A and substituents R^{X11}, R^{X12}, R^{X13} and R^{X14} applied to the structure of A represents any combination described in Combination D (hereinafter, referred to as Compound group SX513).

A compound according to Compound (1V) wherein R¹ represents a methyl group, Q represents Q7, L² represents NH, R^{X9} represents a hydrogen atom, and a combination of structure of A and substituents R^{X11}, R^{X12}, R^{X13} and R^{X14} applied to the structure of A represents any combination described in Combination D (hereinafter, referred to as Compound group SX514).

A compound according to Compound (1V) wherein R¹ represents a methyl group, Q represents Q7, L² represents NH, R^{X9} represents a methyl group, and a combination of structure of A and substituents R^{X11}, R^{X12}, R^{X13} and R^{X14} applied to the structure of A represents any combination described in Combination D (hereinafter, referred to as Compound group SX515).

A compound according to Compound (1V) wherein R¹ represents a chlorine atom, Q represents Q7, L² represents NH, R^{X9} represents a hydrogen atom, and a combination of structure of A and substituents R^{X11}, R^{X12}, R^{X13} and R^{X14} applied to the structure of A represents any combination described in Combination D (hereinafter, referred to as Compound group SX516).

A compound according to Compound (1V) wherein R¹ represents a chlorine atom, Q represents Q7, L² represents NH, R^{X9} represents a methyl group, and a combination of structure of A and substituents R^{X11}, R^{X12}, R^{X13} and R^{X14} applied to the structure of A represents any combination described in Combination D (hereinafter, referred to as Compound group SX517).

A compound according to Compound (1V) wherein R¹ represents a methyl group, Q represents Q8A, R^{X9} represents a hydrogen atom, and a combination of structure of A and substituents R^{X11}, R^{X12}, R^{X13} and R^{X14} applied to the structure of A represents any combination described in Combination D (hereinafter, referred to as Compound group SX518).

A compound according to Compound (1V) wherein R¹ represents a methyl group, Q represents Q8A, R^{X9} represents a methyl group, and a combination of structure of A and substituents R^{X11}, R^{X12}, R^{X13} and R^{X14} applied to the structure of A represents any combination described in Combination D (hereinafter, referred to as Compound group SX519).

A compound according to Compound (1V) wherein R¹ represents a chlorine atom, Q represents Q8A, R^{X9} represents a hydrogen atom, and a combination of structure of A and substituents R^{X11}, R^{X12}, R^{X13} and R^{X14} applied to the structure of A represents any combination described in Combination D (hereinafter, referred to as Compound group SX520).

A compound according to Compound (1V) wherein R¹ represents a chlorine atom, Q represents Q8A, R^{X9} represents a methyl group, and a combination of structure of A and substituents R^{X11}, R^{X12}, R^{X13} and R^{X14} applied to the structure of A represents any combination described in Combination D (hereinafter, referred to as Compound group SX521).

A compound according to Compound (1V) wherein R¹ represents a methyl group, Q represents Q8D, R^{X9} represents a hydrogen atom, and a combination of structure of A and substituents R^{X11}, R^{X12}, R^{X13} and R^{X14} applied to the structure of A represents any combination described in Combination D (hereinafter, referred to as Compound group SX522).

A compound according to Compound (1V) wherein R¹ represents a methyl group, Q represents Q8D, R^{X9} represents a methyl group, and a combination of structure of A and substituents R^{X11}, R^{X12}, R^{X13} and R^{X14} applied to the structure of A represents any combination described in Combination D (hereinafter, referred to as Compound group SX523).

A compound according to Compound (1V) wherein R¹ represents a chlorine atom, Q represents Q8D, R^{X9} represents a hydrogen atom, and a combination of structure of A and substituents R^{X11}, R^{X12}, R^{X13} and R^{X14} applied to the structure of A represents any combination described in Combination D (hereinafter, referred to as Compound group SX524).

A compound according to Compound (1V) wherein R¹ represents a chlorine atom, Q represents Q8D, R^{X9} represents a methyl group, and a combination of structure of A and substituents R^{X11}, R^{X12}, R^{X13} and R^{X14} applied to the structure of A represents any combination described in Combination D (hereinafter, referred to as Compound group SX525).

A compound according to Compound (1V) wherein R¹ represents a methyl group, Q represents Q8J, R^{X9} represents a hydrogen atom, and a combination of structure of A and substituents R^{X11}, R^{X12}, R^{X13} and R^{X14} applied to the structure of A represents any combination described in Combination D (hereinafter, referred to as Compound group SX526).

A compound according to Compound (1V) wherein R¹ represents a methyl group, Q represents Q8J, R^{X9} represents a methyl group, and a combination of structure of A and substituents R^{X11}, R^{X12}, R^{X13} and R^{X14} applied to the structure of A represents any combination described in Combination D (hereinafter, referred to as Compound group SX527).

A compound according to Compound (1V) wherein R¹ represents a chlorine atom, Q represents Q8J, R^{X9} represents a hydrogen atom, and a combination of structure of A and substituents R^{X11}, R^{X12}, R^{X13} and R^{X14} applied to the structure of A represents any combination described in Combination D (hereinafter, referred to as Compound group SX528).

A compound according to Compound (1V) wherein R¹ represents a chlorine atom, Q represents Q8J, R^{X9} represents a methyl group, and a combination of structure of A and substituents R^{X11}, R^{X12}, R^{X13} and R^{X14} applied to the structure of A represents any combination described in Combination D (hereinafter, referred to as Compound group SX529).

A compound represented by formula (1E) (hereinafter, referred to as Compound (1E)):

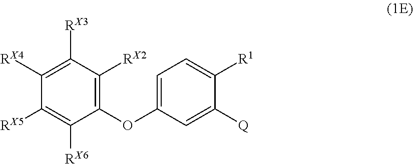

wherein R¹ represents a methyl group, Q represents Q3, R³ represents a methoxy group, and a combination of R^{X2}, R^{X3}, R^{X4}, R^{X5} and R^{X6} represents any combination described in Combination A (hereinafter, referred to as Compound group SX530).

A compound according to Compound (1E) wherein R¹ represents a chlorine atom, Q represents Q3, R³ represents a methoxy group, and a combination of R^{X2}, R^{X3}, R^{X4}, R^{X5} and R^{X6} represents any combination described in Combination A (hereinafter, referred to as Compound group SX531).

A compound according to Compound (1E) wherein R¹ represents a methyl group, Q represents Q4, and a combination of R^{X2}, R^{X3}, R^{X4}, R^{X5} and R^{X6} represents any combination described in Combination A (hereinafter, referred to as Compound group SX532).

A compound according to Compound (1E) wherein R¹ represents a chlorine atom, Q represents Q4, and a combination of R^{X2}, R^{X3}, R^{X4}, R^{X5} and R^{X6} represents any combination described in Combination A (hereinafter, referred to as Compound group SX533).

A compound according to Compound (1E) wherein R¹ represents a methyl group, Q represents Q5, and a combination of R^{X2}, R^{X3}, R^{X4}, R^{X5} and R^{X6} represents any combination described in Combination A (hereinafter, referred to as Compound group SX534).

A compound according to Compound (1E) wherein R¹ represents a chlorine atom, Q represents Q5, and a combination of R^{X2}, R^{X3}, R^{X4}, R^{X5} and R^{X6} represents any combination described in Combination A (hereinafter, referred to as Compound group SX535).

A compound according to Compound (1E) wherein R¹ represents a methyl group, Q represents Q6, and a combination of R^{X2}, R^{X3}, R^{X4}, R^{X5} and R^{X6} represents any combination described in Combination A (hereinafter, referred to as Compound group SX536).

A compound according to Compound (1E) wherein R¹ represents a chlorine atom, Q represents Q6, and a combination of R^{X2}, R^{X3}, R^{X4}, R^{X5} and R^{X6} represents any combination described in Combination A (hereinafter, referred to as Compound group SX537).

A compound according to Compound (1E) wherein R¹ represents a methyl group, Q represents Q7, L² represents an oxygen atom, and a combination of R^{X2}, R^{X3}, R^{X4}, R^{X5} and R^{X6} represents any combination described in Combination A (hereinafter, referred to as Compound group SX538).

A compound according to Compound (1E) wherein R¹ represents a chlorine atom, Q represents Q7, L² represents an oxygen atom, and a combination of R^{X2}, R^{X3}, R^{X4}, R^{X5} and R^{X6} represents any combination described in Combination A (hereinafter, referred to as Compound group SX539).

A compound according to Compound (1E) wherein R¹ represents a methyl group, Q represents Q7, L² represents NH, and a combination of R^{X2}, R^{X3}, R^{X4}, R^{X5} and R^{X6} represents any combination described in Combination A (hereinafter, referred to as Compound group SX540).

A compound according to Compound (1E) wherein R¹ represents a chlorine atom, Q represents Q7, L² represents NH, and a combination of R^{X2}, R^{X3}, R^{X4}, R^{X5} and R^{X6} represents any combination described in Combination A (hereinafter, referred to as Compound group SX541).

A compound according to Compound (1E) wherein R¹ represents a methyl group, Q represents Q8A, and a combination of R^{X2}, R^{X3}, R^{X4}, R^{X5} and R^{X6} represents any combination described in Combination A (hereinafter, referred to as Compound group SX542).

A compound according to Compound (1E) wherein R¹ represents a chlorine atom, Q represents Q8A, and a combination of R^{X2}, R^{X3}, R^{X4}, R^{X5} and R^{X6} represents any combination described in Combination A (hereinafter, referred to as Compound group SX543).

A compound according to Compound (1E) wherein R¹ represents a methyl group, Q represents Q8D, and a combination of R^{X2}, R^{X3}, R^{X4}, R^{X5} and R^{X6} represents any combination described in Combination A (hereinafter, referred to as Compound group SX544).

A compound according to Compound (1E) wherein R¹ represents a chlorine atom, Q represents Q8D, and a combination of R^{X2}, R^{X3}, R^{X4}, R^{X5} and R^{X6} represents any combination described in Combination A (hereinafter, referred to as Compound group SX545).

A compound according to Compound (1E) wherein R¹ represents a methyl group, Q represents Q8J, and a combination of R^{X2}, R^{X3}, R^{X4}, R^{X5} and R^{X6} represents any combination described in Combination A (hereinafter, referred to as Compound group SX546).

A compound according to Compound (1E) wherein R¹ represents a chlorine atom, Q represents Q8J, and a combination of R^{X2}, R^{X3}, R^{X4}, R^{X5} and R^{X6} represents any referred to as Compound group SX547).

A compound according to Compound (1E) wherein R¹ represents a methyl group, Q represents Q1, and a combination of R^{X2}, R^{X3}, R^{X4}, R^{X5} and R^{X6} represents any combination described in Combination H (hereinafter, referred to as Compound group SX548).

Combination H consists of substituent numbers ZH1 to ZH28. Substituent numbers ZH1 to ZH28 represent combinations of substituents R^{X2}, R^{X3}, R^{X4}, R^{X5} and R^{X6} according to Compound (1E). And hereinafter, they are referred to as [substituent number; R^{X2}, R^{X3}, R^{X4}, R^{X5}, R^{X6}]. For example, the substituent number ZH2 refers to a combination wherein R^{X2} represents a methyl group, and each of R^{X3}, R^{X4}, R^{X5} and R^{X6} represents a hydrogen atom.
Combination H: [ZH1;H,H,H,H,H], [ZH2;Me,H,H,H,H], [ZH3;CF₃,H,H,H,H], [ZH4;H,M e,H,H,H], [ZH5;H,OMe,H,H,H], [ZH6;H,OCF₃,H,H,H], [ZH7;H,SMe,H, H,H], [ZH8;H,SCF₃,H,H,H], [ZH9;H,SCHF₂,H,H,H], [ZH10;H,F,H,H,H], [ZH11;H,Br,H,H,H], [ZH12;H,I,H,H,H], [ZH13;H,CN,H,H,H], [ZH1 4;H, t-Bu,H,H,H], [ZH15;H,CCH,H,H,H], [ZH16;H,CCMe,H,H,H], [ZH17;HCC(t-Bu),H,H,H], [ZH18;H,CC(c-Pr),H,H,H], [ZH19;H,CC(SiMe₃),H,H,H], [ZH20;H,H,Me,H,H], [ZH21;H,H,OCF₃,H,H], [ZH22;H,H,SMe,H,H], [ZH23;H,H,SCF₃,H,H], [ZH24;H;HSCHF₂,H,H], [ZH25;H,H,t-Bu,H,H], [ZH26;H,Me,H,Me,H], [ZH27;H,CF₃,H,CF₃,H], [ZH28;H,OPh,H, H, H]

A compound according to Compound (1E) wherein R¹ represents a chlorine atom, Q represents Q1, and a combination of R^{X2}, R^{X3}, R^{X4}, R^{X5} and R^{X6} represents any combination described in Combination H (hereinafter, referred to as Compound group SX549).

A compound according to Compound (1E) wherein R¹ represents a methyl group, Q represents Q2, L¹ represents an oxygen atom, and a combination of R^{X2}, R^{X3}, R^{X4}, R^{X5} and R^{X6} represents any combination described in Combination H (hereinafter, referred to as Compound group SX550).

A compound according to Compound (1E) wherein R¹ represents a chlorine atom, Q represents Q2, L¹ represents an oxygen atom, and a combination of R^{X2}, R^{X3}, R^{X4}, R^{X5} and R^{X6} represents any combination described in Combination H (hereinafter, referred to as Compound group SX551).

A compound according to Compound (1E) wherein R¹ represents a methyl group, Q represents Q2, L¹ represents NH, and a combination of R^{X2}, R^{X3}, R^{X4}, R^{X5} and R^{X6} represents any combination described in Combination H (hereinafter, referred to as Compound group SX552).

A compound according to Compound (1E) wherein R¹ represents a chlorine atom, Q represents Q2, L¹ represents NH, and a combination of R^{X2}, R^{X3}, R^{X4}, R^{X5} and R^{X6} represents any combination described in Combination H (hereinafter, referred to as Compound group SX553).

A compound represented by formula (1F) (hereinafter, referred to as Compound (1F)):

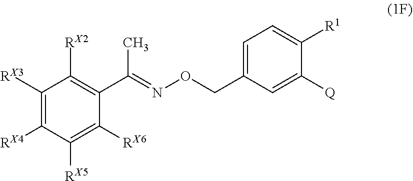

wherein R¹ represents a methyl group, Q represents Q1, and a combination of R^{X2}, R^{X3}, R^{X4}, R^{X5} and R^{X6} represents any combination described in Combination A (hereinafter, referred to as Compound group SX554).

A compound according to Compound (1F) wherein R¹ represents a chlorine atom, Q represents Q1, and a combination of R^{X2}, R^{X3}, R^{X4}, R^{X5} and R^{X6} represents any combination described in Combination A (hereinafter, referred to as Compound group SX555).

A compound according to Compound (1F) wherein R¹ represents an ethyl group, Q represents Q1, and a combination of R^{X2}, R^{X3}, R^{X4}, R^{X5} and R^{X6} represents any combination described in Combination A (hereinafter, referred to as Compound group SX556).

A compound according to Compound (1F) wherein R¹ represents a fluorine atom, Q represents Q1, and a combination of R^{X2}, R^{X3}, R^{X4}, R^{X5} and R^{X6} represents any combination described in Combination A (hereinafter, referred to as Compound group SX557).

A compound according to Compound (1F) wherein R¹ represents a cyclopropyl group, Q represents Q1, and a combination of R^{X2}, R^{X3}, R^{X4}, R^{X5} and R^{X6} represents any combination described in Combination A (hereinafter, referred to as Compound group SX558).

A compound according to Compound (1F) wherein R¹ represents a methyl group, Q represents Q2, L¹ represents an oxygen atom, and a combination of R^{X2}, R^{X3}, R^{X4}, R^{X5} and R^{X6} represents any combination described in Combination A (hereinafter, referred to as Compound group SX559).

A compound according to Compound (1F) wherein R¹ represents a chlorine atom, Q represents Q2, L¹ represents an oxygen atom, and a combination of R^{X2}, R^{X3}, R^{X4}, R^{X5} and R^{X6} represents any combination described in Combination A (hereinafter, referred to as Compound group SX560).

A compound according to Compound (1F) wherein R¹ represents a methyl group, Q represents Q2, L¹ represents NH, and a combination of R^{X2}, R^{X3}, R^{X4}, R^{X5} and R^{X6} represents any combination described in Combination A (hereinafter, referred to as Compound group SX561).

A compound according to Compound (1F) wherein R¹ represents a chlorine atom, Q represents Q2, L¹ represents NH, and a combination of R^{X2}, R^{X3}, R^{X4}, R^{X5} and R^{X6} represents any combination described in Combination A (hereinafter, referred to as Compound group SX562).

A compound according to Compound (1F) wherein R¹ represents a methyl group, Q represents Q3, R³ represents a methoxy group, and a combination of R^{X2}, R^{X3}, R^{X4}, R^{X5} and R^{X6} represents any combination described in Combination A (hereinafter, referred to as Compound group SX563).

A compound according to Compound (1F) wherein R¹ represents a chlorine atom, Q represents Q3, R³ represents a methoxy group, and a combination of R^{X2}, R^{X3}, R^{X4}, R^{X5} and R^{X6} represents any combination described in Combination A (hereinafter, referred to as Compound group SX564).

A compound according to Compound (1F) wherein R¹ represents a methyl group, Q represents Q4, and a combination of R^{X2}, R^{X3}, R^{X4}, R^{X5} and R^{X6} represents any combination described in Combination A (hereinafter, referred to as Compound group SX565).

A compound according to Compound (1F) wherein R¹ represents a chlorine atom, Q represents Q4, and a combination of R^{X2}, R^{X3}, R^{X4}, R^{X5} and R^{X6} represents any combination described in Combination A (hereinafter, referred to as Compound group SX566).

A compound according to Compound (1F) wherein R¹ represents a methyl group, Q represents Q5, and a combination of R^{X2}, R^{X3}, R^{X4}, R^{X5} and R^{X6} represents any combination described in Combination A (hereinafter, referred to as Compound group SX567).

A compound according to Compound (1F) wherein R¹ represents a chlorine atom, Q represents Q5, and a combination of R^{X2}, R^{X3}, R^{X4}, R^{X5} and R^{X6} represents any combination described in Combination A (hereinafter, referred to as Compound group SX568).

A compound according to Compound (1F) wherein R¹ represents a methyl group, Q represents Q6, and a combination of R^{X2}, R^{X3}, R^{X4}, R^{X5} and R^{X6} represents any combination described in Combination A (hereinafter, referred to as Compound group SX569).

A compound according to Compound (1F) wherein R¹ represents a chlorine atom, Q represents Q6, and a combination of R^{X2}, R^{X3}, R^{X4}, R^{X5} and R^{X6} represents any combination described in Combination A (hereinafter, referred to as Compound group SX570).

A compound according to Compound (1F) wherein R¹ represents a methyl group, Q represents Q7, L² represents an oxygen atom, and a combination of R^{X2}, R^{X3}, R^{X4}, R^{X5} and R^{X6} (hereinafter, referred to as Compound group SX571).

A compound according to Compound (1F) wherein R¹ represents a chlorine atom, Q represents Q7, L² represents an oxygen atom, and a combination of R^{X2}, R^{X3}, R^{X4}, R^{X5} and R^{X6} represents any combination described in Combination A (hereinafter, referred to as Compound group SX572).

A compound according to Compound (1F) wherein R¹ represents a methyl group, Q represents Q7, L² represents NH, and a combination of R^{X2}, R^{X3}, R^{X4}, R^{X5} and R^{X6} represents any combination described in Combination A (hereinafter, referred to as Compound group SX573).

A compound according to Compound (1F) wherein R¹ represents a chlorine atom, Q represents Q7, L² represents NH, and a combination of R^{X2}, R^{X3}, R^{X4}, R^{X5} and R^{X6} represents any combination described in Combination A (hereinafter, referred to as Compound group SX574).

A compound according to Compound (1F) wherein R¹ represents a methyl group, Q represents Q8A, and a combination of R^{X2}, R^{X3}, R^{X4}, R^{X5} and R^{X6} represents any combination described in Combination A (hereinafter, referred to as Compound group SX575).

A compound according to Compound (1F) wherein R¹ represents a chlorine atom, Q represents Q8A, and a combination of R^{X2}, R^{X3}, R^{X4}, R^{X5} and R^{X6} represents any combination described in Combination A (hereinafter, referred to as Compound group SX576).

A compound according to Compound (1F) wherein R¹ represents a methyl group, Q represents Q8D, and a combination of R^{X2}, R^{X3}, R^{X4}, R^{X5} and R^{X6} represents any combination described in Combination A (hereinafter, referred to as Compound group SX577).

A compound according to Compound (1F) wherein R¹ represents a chlorine atom, Q represents Q8D, and a combination of R^{X2}, R^{X3}, R^{X4}, R^{X5} and R^{X6} represents any combination described in Combination A (hereinafter, referred to as Compound group SX578).

A compound according to Compound (1F) wherein R¹ represents a methyl group, Q represents Q8J, and a combination of R^{X2}, R^{X3}, R^{X4}, R^{X5} and R^{X6} represents any combination described in Combination A (hereinafter, referred to as Compound group SX579).

A compound according to Compound (1F) wherein R¹ represents a chlorine atom, Q represents Q8J, and a combination of R^{X2}, R^{X3}, R^{X4}, R^{X5} and R^{X6} represents any combination described in Combination A (hereinafter, referred to as Compound group SX580).

A compound represented by formula (1Y) (hereinafter, referred to as Compound (1Y)):

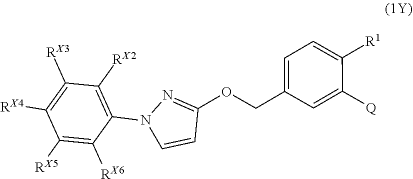

wherein R¹ represents a methyl group, Q represents Q1, and a combination of R^{X2}, R^{X3}, R^{X4}, R^{X5} and R^{X6} represents any combination described in Combination A (hereinafter, referred to as Compound group SX581).

A compound according to Compound (1Y) wherein R¹ represents a chlorine atom, Q represents Q1, and a combination of R^{X2}, R^{X3}, R^{X4}, R^{X5} and R^{X6} represents any combination described in Combination A (hereinafter, referred to as Compound group SX582).

A compound according to Compound (1Y) wherein R¹ represents an ethyl group, Q represents Q1, and a combination of R^{X2}, R^{X3}, R^{X4}, R^{X5} and R^{X6} represents any combination described in Combination A (hereinafter, referred to as Compound group SX583).

A compound according to Compound (1Y) wherein R¹ represents a fluorine atom, Q represents Q1, and a combination of R^{X2}, R^{X3}, R^{X4}, R^{X5} and R^{X6} represents any combination described in Combination A (hereinafter, referred to as Compound group SX584).

A compound according to Compound (1Y) wherein R¹ represents a cyclopropyl group, Q represents Q1, and a combination of R^{X2}, R^{X3}, R^{X4}, R^{X5} and R^{X6} represents any combination described in Combination A (hereinafter, referred to as Compound group SX585).

A compound according to Compound (1Y) wherein R¹ represents a methyl group, Q represents Q2, L¹ represents an oxygen atom, and a combination of R^{X2}, R^{X3}, R^{X4}, R^{X5} and R^{X6} represents any combination described in Combination A (hereinafter, referred to as Compound group SX586).

A compound according to Compound (1Y) wherein R¹ represents a chlorine atom, Q represents Q2, L¹ represents an oxygen atom, and a combination of R^{X2}, R^{X3}, R^{X4}, R^{X5} and R^{X6} represents any combination described in Combination A (hereinafter, referred to as Compound group SX587).

A compound according to Compound (1Y) wherein R¹ represents a methyl group, Q represents Q2, L¹ represents NH, and a combination of R^{X2}, R^{X3}, R^{X4}, R^{X5} and R^{X6} represents any combination described in Combination A (hereinafter, referred to as Compound group SX588).

A compound according to Compound (1Y) wherein R¹ represents a chlorine atom, Q represents Q2, L¹ represents NH, and a combination of R^{X2}, R^{X3}, R^{X4}, R^{X5} and R^{X6} represents any combination described in Combination A (hereinafter, referred to as Compound group SX589).

A compound according to Compound (1Y) wherein R¹ represents a methyl group, Q represents Q3, R³ represents a methoxy group, and a combination of R^{X2}, R^{X3}, R^{X4}, R^{X5} and R^{X6} represents any combination described in Combination A (hereinafter, referred to as Compound group SX590).

A compound according to Compound (1Y) wherein R¹ represents a chlorine atom, Q represents Q3, R³ represents a methoxy group, and a combination of R^{X2}, R^{X3}, R^{X4}, R^{X5} and R^{X6} represents any combination described in Combination A (hereinafter, referred to as Compound group SX591).

A compound according to Compound (1Y) wherein R¹ represents a methyl group, Q represents Q4, and a combination of R^{X2}, R^{X3}, R^{X4}, R^{X5} and R^{X6} represents any combination described in Combination A (hereinafter, referred to as Compound group SX592).

A compound according to Compound (1Y) wherein R¹ represents a chlorine atom, Q represents Q4, and a combination of R^{X2}, R^{X3}, R^{X4}, R^{X5} and R^{X6} represents any combination described in Combination A (hereinafter, referred to as Compound group SX593).

A compound according to Compound (1Y) wherein R¹ represents a methyl group, Q represents Q5, and a combination of R^{X2}, R^{X3}, R^{X4}, R^{X5} and R^{X6} represents any combination described in Combination A (hereinafter, referred to as Compound group SX594).

A compound according to Compound (1Y) wherein R¹ represents a chlorine atom, Q represents Q5, and a combination of R^{X2}, R^{X3}, R^{X4}, R^{X5} and R^{X6} represents any combination described in Combination A (hereinafter, referred to as Compound group SX595).

A compound according to Compound (1Y) wherein R¹ represents a methyl group, Q represents Q6, and a combination of R^{X2}, R^{X3}, R^{X4}, R^{X5} and R^{X6} represents any combination described in Combination A (hereinafter, referred to as Compound group SX596).

A compound according to Compound (1Y) wherein R¹ represents a chlorine atom, Q represents Q6, and a combination of R^{X2}, R^{X3}, R^{X4}, R^{X5} and R^{X6} represents any combination described in Combination A (hereinafter, referred to as Compound group SX597).

A compound according to Compound (1Y) wherein R¹ represents a methyl group, Q represents Q7, L² represents an oxygen atom, and a combination of R^{X2}, R^{X3}, R^{X4}, R^{X5} and R^{X6} represents any combination described in Combination A (hereinafter, referred to as Compound group SX598).

A compound according to Compound (1Y) wherein R¹ represents a chlorine atom, Q represents Q7, L² represents an oxygen atom, and a combination of R^{X2}, R^{X3}, R^{X4}, R^{X5} and R^{X6} represents any combination described in Combination A (hereinafter, referred to as Compound group SX599).

A compound according to Compound (1Y) wherein R¹ represents a methyl group, Q represents Q7, L² represents NH, and a combination of R^{X2}, R^{X3}, R^{X4}, R^{X5} and R^{X6} represents any combination described in Combination A (hereinafter, referred to as Compound group SX600).

A compound according to Compound (1Y) wherein R¹ represents a chlorine atom, Q represents Q7, L² represents NH, and a combination of R^{X2}, R^{X3}, R^{X4}, R^{X5} and R^{X6} represents any combination described in Combination A (hereinafter, referred to as Compound group SX601).

A compound according to Compound (1Y) wherein R¹ represents a methyl group, Q represents Q8A, and a combination of R^{X2}, R^{X3}, R^{X4}, R^{X5} and R^{X6} represents any combination described in Combination A (hereinafter, referred to as Compound group SX602).

A compound according to Compound (1Y) wherein R¹ represents a chlorine atom, Q represents Q8A, and a combination of R^{X2}, R^{X3}, R^{X4}, R^{X5} and R^{X6} represents any combination described in Combination A (hereinafter, referred to as Compound group SX603).

A compound according to Compound (1Y) wherein R¹ represents a methyl group, Q represents Q8D, and a combination of R^{X2}, R^{X3}, R^{X4}, R^{X5} and R^{X6} represents any combination described in Combination A (hereinafter, referred to as Compound group SX604).

A compound according to Compound (1Y) wherein R¹ represents a chlorine atom, Q represents Q8D, and a combination of R^{X2}, R^{X3}, R^{X4}, R^{X5} and R^{X6} represents any combination described in Combination A (hereinafter, referred to as Compound group SX605).

A compound according to Compound (1Y) wherein R¹ represents a methyl group, Q represents Q8J, and a combination of R^{X2}, R^{X3}, R^{X4}, R^{X5} and R^{X6} represents any combination described in Combination A (hereinafter, referred to as Compound group SX606).

A compound according to Compound (1Y) wherein R¹ represents a chlorine atom, Q represents Q8J, and a combination of R^{X2}, R^{X3}, R^{X4}, R^{X5} and R^{X6} represents any combination described in Combination A (hereinafter, referred to as Compound group SX607).

A compound represented by formula (1Z) (hereinafter, referred to as Compound (1Z)):

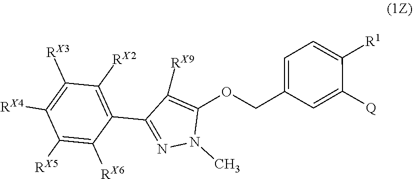

wherein R¹ represents a methyl group, Q represents Q1, R^{X9} represents a methyl group, and a combination of R^{X2}, R^{X3}, R^{X4}, R^{X5} and R^{X6} represents any combination described in Combination A (hereinafter, referred to as Compound group SX608).

A compound according to Compound (1Z) wherein R¹ represents a chlorine atom, Q represents Q1, R^{X9} represents a methyl group, and a combination of R^{X2}, R^{X3}, R^{X4}, R^{X5} and R^{X6} represents any combination described in Combination A (hereinafter, referred to as Compound group SX609).

A compound according to Compound (1Z) wherein R¹ represents an ethyl group, Q represents Q1, R^{X9} represents a methyl group, and a combination of R^{X2}, R^{X3}, R^{X4}, R^{X5} and R^{X6} represents any combination described in Combination A (hereinafter, referred to as Compound group SX610).

A compound according to Compound (1Z) wherein R¹ represents a fluorine atom, Q represents Q1, R^{X9} represents a methyl group, and a combination of R^{X2}, R^{X3}, R^{X4}, R^{X5} and R^{X6} represents any combination described in Combination A (hereinafter, referred to as Compound group SX611).

A compound according to Compound (1Z) wherein R¹ represents a cyclopropyl group, Q represents Q1, R^{X9} represents a methyl group, and a combination of R^{X2}, R^{X3}, R^{X4}, R^{X5} and R^{X6} represents any combination described in Combination A (hereinafter, referred to as Compound group SX612).

A compound according to Compound (1Z) wherein R¹ represents a methyl group, Q represents Q2, L¹ represents an oxygen atom, R^{X9} represents a methyl group, and a combination of R^{X2}, R^{X3}, R^{X4}, R^{X5} and R^{X6} represents any combination described in Combination A (hereinafter, referred to as Compound group SX613).

A compound according to Compound (1Z) wherein R¹ represents a chlorine atom, Q represents Q2, L¹ represents an oxygen atom, R^{X9} represents a methyl group, and a combination of R^{X2}, R^{X3}, R^{X4}, R^{X5} and R^{X6} represents any combination described in Combination A (hereinafter, referred to as Compound group SX614).

A compound according to Compound (1Z) wherein R¹ represents a methyl group, Q represents Q2, L¹ represents NH, R^{X9} represents a methyl group, and a combination of R^{X2}, R^{X3}, R^{X4}, R^{X5} and R^{X6} represents any combination described in Combination A (hereinafter, referred to as Compound group SX615).

A compound according to Compound (1Z) wherein R¹ represents a chlorine atom, Q represents Q2, L¹ represents NH, R^{X9} represents a methyl group, and a combination of R^{X2}, R^{X3}, R^{X4}, R^{X5} and R^{X6} represents any combination described in Combination A (hereinafter, referred to as Compound group SX616).

A compound according to Compound (1Z) wherein R¹ represents a methyl group, Q represents Q3, R³ represents a methoxy group, R^{X9} represents a methyl group, and a combination of R^{X2}, R^{X3}, R^{X4}, R^{X5} and R^{X6} represents any combination described in Combination A (hereinafter, referred to as Compound group SX617).

A compound according to Compound (1Z) wherein R¹ represents a chlorine atom, Q represents Q3, R³ represents a methoxy group, R^{X9} represents a methyl group, and a combination of R^{X2}, R^{X3}, R^{X4}, R^{X5} and R^{X6} represents any combination described in Combination A (hereinafter, referred to as Compound group SX618).

A compound according to Compound (1Z) wherein R¹ represents a methyl group, Q represents Q4, R^{X9} represents a methyl group, and a combination of R^{X2}, R^{X3}, R^{X4}, R^{X5} and R^{X6} represents any combination described in Combination A (hereinafter, referred to as Compound group SX619).

A compound according to Compound (1Z) wherein R¹ represents a chlorine atom, Q represents Q4, R^{X9} represents a methyl group, and a combination of R^{X2}, R^{X3}, R^{X4}, R^{X5} and R^{X6} represents any combination described in Combination A (hereinafter, referred to as Compound group SX620).

A compound according to Compound (1Z) wherein R¹ represents a methyl group, Q represents Q5, R^{X9} represents a methyl group, and a combination of R^{X2}, R^{X3}, R^{X4}, R^{X5} and R^{X6} represents any combination described in Combination A (hereinafter, referred to as Compound group SX621).

A compound according to Compound (1Z) wherein R¹ represents a chlorine atom, Q represents Q5, R^{X9} represents a methyl group, and a combination of R^{X2}, R^{X3}, R^{X4}, R^{X5} and R^{X6} (hereinafter, referred to as Compound group SX622).

A compound according to Compound (1Z) wherein R¹ represents a methyl group, Q represents Q6, R^{X9} represents a methyl group, and a combination of R^{X2}, R^{X3}, R^{X4}, R^{X5} and R^{X6} represents any combination described in Combination A (hereinafter, referred to as Compound group SX623).

A compound according to Compound (1Z) wherein R¹ represents a chlorine atom, Q represents Q6, R^{X9} represents a methyl group, and a combination of R^{X2}, R^{X3}, R^{X4}, R^{X5} and R^{X6} represents any combination described in Combination A (hereinafter, referred to as Compound group SX624).

A compound according to Compound (1Z) wherein R¹ represents a methyl group, Q represents Q7, L² represents an oxygen atom, R^{X9} represents a methyl group, and a combination of R^{X2}, R^{X3}, R^{X4}, R^{X5} and R^{X6} represents any combination described in Combination A (hereinafter, referred to as Compound group SX625).

A compound according to Compound (1Z) wherein R¹ represents a chlorine atom, Q represents Q7, L² represents an oxygen atom, R^{X9} represents a methyl group, and a combination of R^{X2}, R^{X3}, R^{X4}, R^{X5} and R^{X6} represents any combination described in Combination A (hereinafter, referred to as Compound group SX626).

A compound according to Compound (1Z) wherein R¹ represents a methyl group, Q represents Q7, L² represents NH, R^{X9} represents a methyl group, and a combination of R^{X2}, R^{X3}, R^{X4}, R^{X5} and R^{X6} represents any combination described in Combination A (hereinafter, referred to as Compound group SX627).

A compound according to Compound (1Z) wherein R¹ represents a chlorine atom, Q represents Q7, L² represents NH, R^{X9} represents a methyl group, and a combination of R^{X2}, R^{X3}, R^{X4}, R^{X5} and R^{X6} represents any combination described in Combination A (hereinafter, referred to as Compound group SX628).

A compound according to Compound (1Z) wherein R¹ represents a methyl group, Q represents Q8A, R^{X9} represents a methyl group, and a combination of R^{X2}, R^{X3}, R^{X4}, R^{X5} and R^{X6} represents any combination described in Combination A (hereinafter, referred to as Compound group SX629).

A compound according to Compound (1Z) wherein R¹ represents a chlorine atom, Q represents Q8A, R^{X9} represents a methyl group, and a combination of R^{X2}, R^{X3}, R^{X4}, R^{X5} and R^{X6} represents any combination described in Combination A (hereinafter, referred to as Compound group SX630).

A compound according to Compound (1Z) wherein R¹ represents a methyl group, Q represents Q8D, R^{X9} represents a methyl group, and a combination of R^{X2}, R^{X3}, R^{X4}, R^{X5} and R^{X6} represents any combination described in Combination A (hereinafter, referred to as Compound group SX631).

A compound according to Compound (1Z) wherein R¹ represents a chlorine atom, Q represents Q8D, R^{X9} represents a methyl group, and a combination of R^{X2}, R^{X3}, R^{X4}, R^{X5} and R^{X6} represents any combination described in Combination A (hereinafter, referred to as Compound group SX632).

A compound according to Compound (1Z) wherein R¹ represents a methyl group, Q represents Q8J, R^{X9} represents a methyl group, and a combination of R^{X2}, R^{X3}, R^{X4}, R^{X5} and R^{X6} represents any combination described in Combination A (hereinafter, referred to as Compound group SX633).

A compound according to Compound (1Z) wherein R¹ represents a chlorine atom, Q represents Q8J, R^{X9} represents a methyl group, and a combination of R^{X2}, R^{X3}, R^{X4}, R^{X5} and R^{X6} represents any combination described in Combination A (hereinafter, referred to as Compound group SX634).

A compound according to Compound (1Z) wherein R¹ represents a methyl group, Q represents Q1, R^{X9} represents a hydrogen atom, and a combination of R^{X2}, R^{X3}, R^{X4}, R^{X5} and R^{X6} represents any combination described in Combination A (hereinafter, referred to as Compound group SX635).

A compound according to Compound (1Z) wherein R¹ represents a chlorine atom, Q represents Q1, R^{X9} represents a hydrogen atom, and a combination of R^{X2}, R^{X3}, R^{X4}, R^{X5} and R^{X6} represents any combination described in Combination A (hereinafter, referred to as Compound group SX636).

A compound according to Compound (1Z) wherein R¹ represents an ethyl group, Q represents Q1, R^{X9} represents a hydrogen atom, and a combination of R^{X2}, R^{X3}, R^{X4}, R^{X5} and R^{X6} represents any combination described in Combination A (hereinafter, referred to as Compound group SX637).

A compound according to Compound (1Z) wherein R¹ represents a fluorine atom, Q represents Q1, R^{X9} represents a hydrogen atom, and a combination of R^{X2}, R^{X3}, R^{X4}, R^{X5} and R^{X6} represents any combination described in Combination A (hereinafter, referred to as Compound group SX638).

A compound according to Compound (1Z) wherein R¹ represents a cyclopropyl group, Q represents Q1, R^{X9} represents a hydrogen atom, and a combination of R^{X2}, R^{X3}, R^{X4}, R^{X5} and R^{X6} represents any combination described in Combination A (hereinafter, referred to as Compound group SX639).

A compound according to Compound (1Z) wherein R¹ represents a methyl group, Q represents Q2, L¹ represents an oxygen atom, R^{X9} represents a hydrogen atom, and a combination of R^{X2}, R^{X3}, R^{X4}, R^{X5} and R^{X6} represents any combination described in Combination A (hereinafter, referred to as Compound group SX640).

A compound according to Compound (1Z) wherein R¹ represents a chlorine atom, Q represents Q2, L¹ represents an oxygen atom, R^{X9} represents a hydrogen atom, and a combination of R^{X2}, R^{X3}, R^{X4}, R^{X5} and R^{X6} represents any combination described in Combination A (hereinafter, referred to as Compound group SX641).

A compound according to Compound (1Z) wherein R¹ represents a methyl group, Q represents Q2, L¹ represents NH, R^{X9} represents a hydrogen atom, and a combination of R^{X2}, R^{X3}, R^{X4}, R^{X5} and R^{X6} represents any combination described in Combination A (hereinafter, referred to as Compound group SX642).

A compound according to Compound (1Z) wherein R¹ represents a chlorine atom, Q represents Q2, L¹ represents NH, R^{X9} represents a hydrogen atom, and a combination of R^{X2}, R^{X3}, R^{X4}, R^{X5} and R^{X6} represents any combination described in Combination A (hereinafter, referred to as Compound group SX643).

A compound according to Compound (1Z) wherein R¹ represents a methyl group, Q represents Q3, R³ represents a methoxy group, R^{X9} represents a hydrogen atom, and a combination of R^{X2}, R^{X3}, R^{X4}, R^{X5} and R^{X6} represents any combination described in Combination A (hereinafter, referred to as Compound group SX644).

A compound according to Compound (1Z) wherein R¹ represents a chlorine atom, Q represents Q3, R³ represents a methoxy group, R^{X9} represents a hydrogen atom, and a combination of R^{X2}, R^{X3}, R^{X4}, R^{X5} and R^{X6} represents any combination described in Combination A (hereinafter, referred to as Compound group SX645).

A compound according to Compound (1Z) wherein R¹ represents a methyl group, Q represents Q4, R^{X9} represents a hydrogen atom, and a combination of R^{X2}, R^{X3}, R^{X4}, R^{X5} and R^{X6} represents any combination described in Combination A (hereinafter, referred to as Compound group SX646).

A compound according to Compound (1Z) wherein R¹ represents a chlorine atom, Q represents Q4, R^{X9} represents a hydrogen atom, and a combination of R^{X2}, R^{X3}, R^{X4}, R^{X5} and R^{X6} represents any combination described in Combination A (hereinafter, referred to as Compound group SX647).

A compound according to Compound (1Z) wherein R¹ represents a methyl group, Q represents Q5, R^{X9} represents a hydrogen atom, and a combination of R^{X2}, R^{X3}, R^{X4}, R^{X5} and R^{X6} represents any combination described in Combination A (hereinafter, referred to as Compound group SX648).

A compound according to Compound (1Z) wherein R¹ represents a chlorine atom, Q represents Q5, R^{X9} represents a hydrogen atom, and a combination of R^{X2}, R^{X3}, R^{X4}, R^{X5} and R^{X6} represents any combination described in Combination A (hereinafter, referred to as Compound group SX649).

A compound according to Compound (1Z) wherein R¹ represents a methyl group, Q represents Q6, R^{X9} represents a hydrogen atom, and a combination of R^{X2}, R^{X3}, R^{X4}, R^{X5} and R^{X6} represents any combination described in Combination A (hereinafter, referred to as Compound group SX650).

A compound according to Compound (1Z) wherein R¹ represents a chlorine atom, Q represents Q6, R^{X9} represents a hydrogen atom, and a combination of R^{X2}, R^{X3}, R^{X4}, R^{X5} and R^{X6} represents any combination described in Combination A (hereinafter, referred to as Compound group SX651).

A compound according to Compound (1Z) wherein R¹ represents a methyl group, Q represents Q7, L² represents an oxygen atom, R^{X9} represents a hydrogen atom, and a combination of R^{X2}, R^{X3}, R^{X4}, R^{X5} and R^{X6} represents any combination described in Combination A (hereinafter, referred to as Compound group SX652).

A compound according to Compound (1Z) wherein R¹ represents a chlorine atom, Q represents Q7, L² represents an oxygen atom, R^{X9} represents a hydrogen atom, and a combination of R^{X2}, R^{X3}, R^{X4}, R^{X5} and R^{X6} represents any combination described in Combination A (hereinafter, referred to as Compound group SX653).

A compound according to Compound (1Z) wherein R¹ represents a methyl group, Q represents Q7, L² represents NH, R^{X9} represents a hydrogen atom, and a combination of R^{X2}, R^{X3}, R^{X4}, R^{X5} and R^{X6} represents any combination described in Combination A (hereinafter, referred to as Compound group SX654).

A compound according to Compound (1Z) wherein R¹ represents a chlorine atom, Q represents Q7, L² represents NH, R^{X9} represents a hydrogen atom, and a combination of R^{X2}, R^{X3}, R^{X4}, R^{X5} and R^{X6} represents any combination described in Combination A (hereinafter, referred to as Compound group SX655).

A compound according to Compound (1Z) wherein R¹ represents a methyl group, Q represents Q8A, R^{X9} represents a hydrogen atom, and a combination of R^{X2}, R^{X3}, R^{X4}, R^{X5} and R^{X6} represents any combination described in Combination A (hereinafter, referred to as Compound group SX656).

A compound according to Compound (1Z) wherein R¹ represents a chlorine atom, Q represents Q8A, R^{X9} represents a hydrogen atom, and a combination of R^{X2}, R^{X3}, R^{X4}, R^{X5} and R^{X6} represents any combination described in Combination A (hereinafter, referred to as Compound group SX657).

A compound according to Compound (1Z) wherein R¹ represents a methyl group, Q represents Q8D, R^{X9} represents a hydrogen atom, and a combination of R^{X2}, R^{X3}, R^{X4}, R^{X5} and R^{X6} represents any combination described in Combination A (hereinafter, referred to as Compound group SX658).

A compound according to Compound (1Z) wherein R¹ represents a chlorine atom, Q represents Q8D, R^{X9} represents a hydrogen atom, and a combination of R^{X2}, R^{X3}, R^{X4}, R^{X5} and R^{X6} represents any combination described in Combination A (hereinafter, referred to as Compound group SX659).

A compound according to Compound (1Z) wherein R¹ represents a methyl group, Q represents Q8J, R^{X9} represents a hydrogen atom, and a combination of R^{X2}, R^{X3}, R^{X4}, R^{X5} and R^{X6} represents any combination described in Combination A (hereinafter, referred to as Compound group SX660).

A compound according to Compound (1Z) wherein R¹ represents a chlorine atom, Q represents Q8J, R^{X9} represents a hydrogen atom, and a combination of R^{X2}, R^{X3}, R^{X4}, R^{X5} and R^{X6} represents any combination described in Combination A (hereinafter, referred to as Compound group SX661).

A compound represented by formula (1W) (hereinafter, referred to as Compound (1W)):

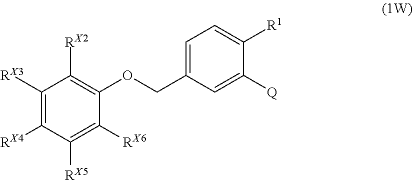

wherein R¹ represents a methyl group, Q represents Q1, and a combination of R^{X2}, R^{X3}, R^{X4}, R^{X5} and R^{X6} represents any combination described in Combination A (hereinafter, referred to as Compound group SX662).

A compound according to Compound (1W) wherein R¹ represents a chlorine atom, Q represents Q1, and a combination of R^{X2}, R^{X3}, R^{X4}, R^{X5} and R^{X6} represents any combination described in Combination A (hereinafter, referred to as Compound group SX663).

A compound according to Compound (1W) wherein R¹ represents an ethyl group, Q represents Q1, and a combination of R^{X2}, R^{X3}, R^{X4}, R^{X5} and R^{X6} represents any combination described in Combination A (hereinafter, referred to as Compound group SX664).

A compound according to Compound (1W) wherein R¹ represents a fluorine atom, Q represents Q1, and a combination of R^{X2}, R^{X3}, R^{X4}, R^{X5} and R^{X6} represents any combination described in Combination A (hereinafter, referred to as Compound group SX665).

A compound according to Compound (1W) wherein R¹ represents a cyclopropyl group, Q represents Q1, and a combination of R^{X2}, R^{X3}, R^{X4}, R^{X5} and R^{X6} represents any combination described in Combination A (hereinafter, referred to as Compound group SX666).

A compound according to Compound (1W) wherein R¹ represents a methyl group, Q represents Q2, L¹ represents an oxygen atom, and a combination of R^{X2}, R^{X3}, R^{X4}, R^{X5} and R^{X6} represents any combination described in Combination A (hereinafter, referred to as Compound group SX667).

A compound according to Compound (1W) wherein R¹ represents a chlorine atom, Q represents Q2, L¹ represents an oxygen atom, and a combination of R^{X2}, R^{X3}, R^{X4}, R^{X5} and R^{X6} represents any combination described in Combination A (hereinafter, referred to as Compound group SX668).

A compound according to Compound (1W) wherein R¹ represents a methyl group, Q represents Q2, L¹ represents NH, and a combination of R^{X2}, R^{X3}, R^{X4}, R^{X5} and R^{X6} represents any combination described in Combination A (hereinafter, referred to as Compound group SX669).

A compound according to Compound (1W) wherein R¹ represents a chlorine atom, Q represents Q2, L¹ represents NH, and a combination of R^{X2}, R^{X3}, R^{X4}, R^{X5} and R^{X6} represents any combination described in Combination A (hereinafter, referred to as Compound group SX670).

A compound according to Compound (1W) wherein R¹ represents a methyl group, Q represents Q3, R³ represents a methoxy group, and a combination of R^{X2}, R^{X3}, R^{X4}, R^{X5} and R^{X6} represents any combination described in Combination A (hereinafter, referred to as Compound group SX671).

A compound according to Compound (1W) wherein R¹ represents a chlorine atom, Q represents Q3, R³ represents a methoxy group, and a combination of R^{X2}, R^{X3}, R^{X4}, R^{X5} and R^{X6} represents any combination described in Combination A (hereinafter, referred to as Compound group SX672).

A compound according to Compound (1W) wherein R¹ represents a methyl group, Q represents Q4, and a combination of R^{X2}, R^{X3}, R^{X4}, R^{X5} and R^{X6} represents any combination described in Combination A (hereinafter, referred to as Compound group SX673).

A compound according to Compound (1W) wherein R¹ represents a chlorine atom, Q represents Q4, and a combination of R^{X2}, R^{X3}, R^{X4}, R^{X5} and R^{X6} represents any combination described in Combination A (hereinafter, referred to as Compound group SX674).

A compound according to Compound (1W) wherein R¹ represents a methyl group, Q represents Q5, and a combination of R^{X2}, R^{X3}, R^{X4}, R^{X5} and R^{X6} represents any combination described in Combination A (hereinafter, referred to as Compound group SX675).

A compound according to Compound (1W) wherein R¹ represents a chlorine atom, Q represents Q5, and a combination of R^{X2}, R^{X3}, R^{X4}, R^{X5} and R^{X6} represents any combination described in Combination A (hereinafter, referred to as Compound group SX676).

A compound according to Compound (1W) wherein R¹ represents a methyl group, Q represents Q6, and a combination of R^{X2}, R^{X3}, R^{X4}, R^{X5} and R^{X6} represents any combination described in Combination A (hereinafter, referred to as Compound group SX677).

A compound according to Compound (1W) wherein R¹ represents a chlorine atom, Q represents Q6, and a combination of R^{X2}, R^{X3}, R^{X4}, R^{X5} and R^{X6} represents any combination described in Combination A (hereinafter, referred to as Compound group SX678).

A compound according to Compound (1W) wherein R¹ represents a methyl group, Q represents Q7, L² represents an oxygen atom, and a combination of R^{X2}, R^{X3}, R^{X4}, R^{X5} and R^{X6} represents any combination described in Combination A (hereinafter, referred to as Compound group SX679).

A compound according to Compound (1W) wherein R¹ represents a chlorine atom, Q represents Q7, L² represents an oxygen atom, and a combination of R^{X2}, R^{X3}, R^{X4}, R^{X5} and R^{X6} represents any combination described in Combination A (hereinafter, referred to as Compound group SX680).

A compound according to Compound (1W) wherein R¹ represents a methyl group, Q represents Q7, L² represents NH, and a combination of R^{X2}, R^{X3}, R^{X4}, R^{X5} and R^{X6} represents any combination described in Combination A (hereinafter, referred to as Compound group SX681).

A compound according to Compound (1W) wherein R¹ represents a chlorine atom, Q represents Q7, L² represents NH, and a combination of R^{X2}, R^{X3}, R^{X4}, R^{X5} and R^{X6} represents any combination described in Combination A (hereinafter, referred to as Compound group SX682).

A compound according to Compound (1W) wherein R¹ represents a methyl group, Q represents Q8A, and a combination of R^{X2}, R^{X3}, R^{X4}, R^{X5} and R^{X6} represents any combination described in Combination A (hereinafter, referred to as Compound group SX683).

A compound according to Compound (1W) wherein R¹ represents a chlorine atom, Q represents Q8A, and a combination of R^{X2}, R^{X3}, R^{X4}, R^{X5} and R^{X6} represents any combination described in Combination A (hereinafter, referred to as Compound group SX684).

A compound according to Compound (1W) wherein R¹ represents a methyl group, Q represents Q8D, and a combination of R^{X2}, R^{X3}, R^{X4}, R^{X5} and R^{X6} represents any combination described in Combination A (hereinafter, referred to as Compound group SX685).

A compound according to Compound (1W) wherein R¹ represents a chlorine atom, Q represents Q8D, and a combination of R^{X2}, R^{X3}, R^{X4}, R^{X5} and R^{X6} represents any combination described in Combination A (hereinafter, referred to as Compound group SX686).

A compound according to Compound (1W) wherein R¹ represents a methyl group, Q represents Q8J, and a combination of R^{X2}, R^{X3}, R^{X4}, R^{X5} and R^{X6} represents any combination described in Combination A (hereinafter, referred to as Compound group SX687).

A compound according to Compound (1W) wherein R¹ represents a chlorine atom, Q represents Q8J, and a combination of R^{X2}, R^{X3}, R^{X4}, R^{X5} and R^{X6} represents any combination described in Combination A (hereinafter, referred to as Compound group SX688].

A compound represented by formula (1X) (hereinafter, referred to as Compound (1X)):

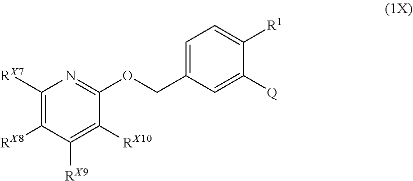

wherein R¹ represents a methyl group, Q represents Q1, and a combination of R^{X7}, R^{X8}, R^{X9} and R^{X10} represents any combination described in Combination C (hereinafter, referred to as Compound group SX689).

Combination C consists of substituent numbers ZC1 to ZC57. Substituent numbers ZC1 to ZC57 represent combinations of R^{X7}, R^{X8}, R^{X9} and R^{X10} according to Compound (1X). And hereinafter, they are referred to as [substituent number; R^{X7}, R^{X8}, R^{X9}, R^{X10}]. For example, the substituent number ZC2 refers to a combination wherein R^{X7} represents a methyl group, and each of R^{X8}, R^{X9} and R^{X10} represents a hydrogen atom.
Combination C: [ZC1;H,H,H,H], [ZC2;Me,H,H,H], [ZC3;Et,H,H,H], [ZC4;Pr,H,H,H], [ZC5;i-Pr,H,H,H], [ZC6;c-Pr,H,H,H], [ZC7;Ph,H,H,H], [ZC8;OMe,H,H,H], [ZC9;OEt,H,H,H], [Z C10;OPh,H,H,H], [ZC11;CF₃,H,H,H], [ZC12;F,H,H,H], [ZC13;Cl,H,H,H], [ZC14;Br,H,H,H], [ZC15;CN,H,H,H], [ZC16;H,Me,H,H], [ZC17;H,Et,H,H], [ZC18;H,Pr,H,H], [ZC19;H,i-Pr,H,H], [ZC20;H,c-Pr,H,H], [ZC21;H,Ph,H,H], [ZC22;H,OMe,H,H], [ZC23;H,OEt,H,H], [ZC24;H,OPh,H,H], [ZC25;H,CF₃,H,H], [ZC26;H,F,H,H], [ZC27;H,Cl, H,H], [ZC28;H,Br,H,H], [ZC29;H,CN,H,H], [ZC30;H,H,Me,H], [ZC31; H,H,Et,H], [ZC32;H,H,Pr,H], [ZC33;H,H,i-Pr,H], [ZC34;H,H,c-Pr,H], [ZC35;H,H,Ph,H], [ZC36;H,H,OMe,H], [ZC37;H,H,OEt,H], [ZC 38;H,H,OPh,H], [ZC39;H,H,CF₃,H], [ZC40;H,H,F,H], [ZC41;H,H,Cl, H], [ZC42;H,H,Br,H], [ZC43;H,H,CN,H], [ZC44;H,H,H,Me], [ZC45;H, H,H,Et], [ZC46;H,H,H,Pr], [ZC47;H,H,H,i-Pr], [ZC48;H,H,H,c-Pr], [ZC49;H,H,H,Ph], [ZC50;H,H,H,OMe], [ZC51;H,H,H,OEt], [ZC52;H,H,H,OPh], [ZC53;H,H,H,CF₃], [ZC54;H,H,H,F], [ZC55;H,H,H,Cl], [ZC56;H,H,H,Br], [ZC57;H,H,H,CN]

A compound according to Compound (1X) wherein R¹ represents a chlorine atom, Q represents Q1, and a combination of R^{X7}, R^{X8}, R^{X9} and R^{X10} represents any combination described in Combination C (hereinafter, referred to as Compound group SX690).

A compound according to Compound (1X) wherein R¹ represents an ethyl group, Q represents Q1, and a combination of R^{X7}, R^{X8}, R^{X9} and R^{X10} represents any combination described in Combination C (hereinafter, referred to as Compound group SX691).

A compound according to Compound (1X) wherein R¹ represents a fluorine atom, Q represents Q1, and a combination of R^{X7}, R^{X8}, R^{X9} and R^{X10} represents any combination described in Combination C (hereinafter, referred to as Compound group SX692).

A compound according to Compound (1X) wherein R¹ represents a cyclopropyl group, Q represents Q1, and a combination of R^{X7}, R^{X8}, R^{X9} and R^{X10} represents any combination described in Combination C (hereinafter, referred to as Compound group SX693).

A compound according to Compound (1X) wherein R¹ represents a methyl group, Q represents Q2, L¹ represents an oxygen atom, and a combination of R^{X7}, R^{X8}, R^{X9} and R^{X10} represents any combination described in Combination C (hereinafter, referred to as Compound group SX694).

A compound according to Compound (1X) wherein R¹ represents a chlorine atom, Q represents Q2, L¹ represents an oxygen atom, and a combination of R^{X7}, R^{X8}, R^{X9} and R^{X10} represents any combination described in Combination C (hereinafter, referred to as Compound group SX695).

A compound according to Compound (1X) wherein R¹ represents a methyl group, Q represents Q2, L¹ represents NH, and a combination of R^{X7}, R^{X8}, R^{X9} and R^{X10} represents any combination described in Combination C (hereinafter, referred to as Compound group SX696).

A compound according to Compound (1X) wherein R¹ represents a chlorine atom, Q represents Q2, L¹ represents NH, and a combination of R^{X7}, R^{X8}, R^{X9} and R^{X10} represents any combination described in Combination C (hereinafter, referred to as Compound group SX697).

A compound according to Compound (1X) wherein R¹ represents a methyl group, Q represents Q3, R³ represents a methoxy group, and a combination of R^{X7}, R^{X8}, R^{X9} and R^{X10} represents any combination described in Combination C (hereinafter, referred to as Compound group SX698).

A compound according to Compound (1X) wherein R¹ represents a chlorine atom, Q represents Q3, R³ represents a methoxy group, and a combination of R^{X7}, R^{X8}, R^{X9} and R^{X10} represents any combination described in Combination C (hereinafter, referred to as Compound group SX699).

A compound according to Compound (1X) wherein R¹ represents a methyl group, Q represents Q4, and a combination of R^{X7}, R^{X8}, R^{X9} and R^{X10} represents any combination described in Combination C (hereinafter, referred to as Compound group SX700).

A compound according to Compound (1X) wherein R¹ represents a chlorine atom, Q represents Q4, and a combination of R^{X7}, R^{X8}, R^{X9} and R^{X10} represents any combination described in Combination C (hereinafter, referred to as Compound group SX701).

A compound according to Compound (1X) wherein R¹ represents a methyl group, Q represents Q5, and a combination of R^{X7}, R^{X8}, R^{X9} and R^{X10} represents any combination described in Combination C (hereinafter, referred to as Compound group SX702).

A compound according to Compound (1X) wherein R¹ represents a chlorine atom, Q represents Q5, and a combination of R^{X7}, R^{X8}, R^{X9} and R^{X10} represents any combination described in Combination C (hereinafter, referred to as Compound group SX703).

A compound according to Compound (1X) wherein R¹ represents a methyl group, Q represents Q6, and a combination of R^{X7}, R^{X8}, R^{X9} and R^{X10} represents any combination described in Combination C (hereinafter, referred to as Compound group SX704).

A compound according to Compound (1X) wherein R¹ represents a chlorine atom, Q represents Q6, and a combination of R^{X7}, R^{X5}, R^{X9} and R^{X10} represents any combination described in Combination C (hereinafter, referred to as Compound group SX705).

A compound according to Compound (1X) wherein R¹ represents a methyl group, Q represents Q7, L² represents an oxygen atom, and a combination of R^{X7}, R^{X8}, R^{X9} and R^{X10} represents any combination described in Combination C (hereinafter, referred to as Compound group SX706).

A compound according to Compound (1X) wherein R¹ represents a chlorine atom, Q represents Q7, L² represents an oxygen atom, and a combination of R^{X7}, R^{X8}, R^{X9} and R^{X10} represents any combination described in Combination C (hereinafter, referred to as Compound group SX707).

A compound according to Compound (1X) wherein R¹ represents a methyl group, Q represents Q7, L² represents NH, and a combination of R^{X7}, R^{X8}, R^{X9} and R^{X10} represents any combination described in Combination C (hereinafter, referred to as Compound group SX708).

A compound according to Compound (1X) wherein R¹ represents a chlorine atom, Q represents Q7, L² represents NH, and a combination of R^{X7}, R^{X8}, R^{X9} and R^{X10} represents any combination described in Combination C (hereinafter, referred to as Compound group SX709).

A compound according to Compound (1X) wherein R¹ represents a methyl group, Q represents Q8A, and a combination of R^{X7}, R^{X8}, R^{X9} and R^{X10} represents any combination described in Combination C (hereinafter, referred to as Compound group SX710).

A compound according to Compound (1X) wherein R¹ represents a chlorine atom, Q represents Q8A, and a combination of R^{X7}, R^{X8}, R^{X9} and R^{X10} represents any combination described in Combination C (hereinafter, referred to as Compound group SX711).

A compound according to Compound (1X) wherein R¹ represents a methyl group, Q represents Q8D, and a combination of R^{X7}, R^{X8}, R^{X9} and R^{X10} represents any combination described in Combination C (hereinafter, referred to as Compound group SX712).

A compound according to Compound (1X) wherein R¹ represents a chlorine atom, Q represents Q8D, and a combination of R^{X7}, R^{X8}, R^{X9} and R^{X10} represents any combination described in Combination C (hereinafter, referred to as Compound group SX713).

A compound according to Compound (1X) wherein R¹ represents a methyl group, Q represents Q8J, and a combination of R^{X7}, R^{X8}, R^{X9} and R^{X10} represents any combination described in Combination C (hereinafter, referred to as Compound group SX714).

A compound according to Compound (1X) wherein R¹ represents a chlorine atom, Q represents Q8J, and a combination of R^{X7}, R^{X8}, R^{X9} and R^{X10} represents any combination described in Combination C (hereinafter, referred to as Compound group SX715).

A compound represented by formula (2B) (hereinafter, referred to as Compound (2B)):

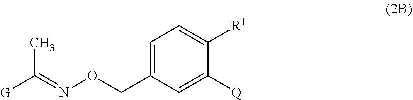

[wherein G represents any one of formulae G1 to G35.]
wherein R¹ represents a methyl group, Q represents Q1, and a combination of structure of G and substituents R^{X7}, R^{X8}, R^{X9} and R^{X10} applied to the structure of G represents any combination described in Combination B (hereinafter, referred to as Compound group SX716).

A compound according to Compound (2B) wherein R¹ represents a chlorine atom, Q represents Q1, and a combination of structure of G and substituents R^{X7}, R^{X8}, R^{X9} and R^{X10} applied to the structure of G represents any combination described in Combination B (hereinafter, referred to as Compound group SX717).

A compound according to Compound (2B) wherein R¹ represents a methyl group, Q represents Q2, L represents an oxygen atom, and a combination of structure of G and substituents R^{X7}, R^{X8}, R^{X9} and R^{X10} applied to the structure of G represents any combination described in Combination B (hereinafter, referred to as Compound group SX718).

A compound according to Compound (2B) wherein R¹ represents a chlorine atom, Q represents Q2, L represents an oxygen atom, and a combination of structure of G and substituents R^{X7}, R^{X8}, R^{X9} and R^{X10} applied to the structure of G represents any combination described in Combination B (hereinafter, referred to as Compound group SX719).

A compound according to Compound (2B) wherein R¹ represents a methyl group, Q represents Q2, L represents NH, and a combination of structure of G and substituents R^{X7}, R^{X8}, R^{X9} and R^{X10} applied to the structure of G represents any combination described in Combination B (hereinafter, referred to as Compound group SX720).

A compound according to Compound (2B) wherein R¹ represents a chlorine atom, Q represents Q2, L represents NH, and a combination of structure of G and substituents R^{X7}, R^{X8}, R^{X9} and R^{X10} applied to the structure of G represents any combination described in Combination B (hereinafter, referred to as Compound group SX721).

A compound according to Compound (2B) wherein R¹ represents a methyl group, Q represents Q3, R³ represents a methoxy group, and a combination of structure of G and substituents R^{X7}, R^{X8}, R^{X9} and R^{X10} applied to the structure of G represents any combination described in Combination B (hereinafter, referred to as Compound group SX722).

A compound according to Compound (2B) wherein R¹ represents a chlorine atom, Q represents Q3, R³ represents a methoxy group, and a combination of structure of G and substituents R^{X7}, R^{X8}, R^{X9} and R^{X10} applied to the structure of G represents any combination described in Combination B (hereinafter, referred to as Compound group SX723).

A compound according to Compound (2B) wherein R¹ represents a methyl group, Q represents Q4, and a combination of structure of G and substituents R^{X7}, R^{X8}, R^{X9} and R^{X10} applied to the structure of G represents any combination described in Combination B (hereinafter, referred to as Compound group SX724).

A compound according to Compound (2B) wherein R¹ represents a chlorine atom, Q represents Q4, and a combination of structure of G and substituents R^{X7}, R^{X8}, R^{X9} and R^{X10} applied to the structure of G represents any combination described in Combination B (hereinafter, referred to as Compound group SX725).

A compound according to Compound (2B) wherein R¹ represents a methyl group, Q represents Q5, and a combination of structure of G and substituents R^{X7}, R^{X8}, R^{X9} and R^{X10} applied to the structure of G represents any combination described in Combination B (hereinafter, referred to as Compound group SX726).

A compound according to Compound (2B) wherein R¹ represents a chlorine atom, Q represents Q5, and a combination of structure of G and substituents R^{X7}, R^{X8}, R^{X9} and R^{X10} applied to the structure of G represents any combination described in Combination B (hereinafter, referred to as Compound group SX727).

A compound according to Compound (2B) wherein R¹ represents a methyl group, Q represents Q6, and a combination of structure of G and substituents R^{X7}, R^{X8}, R^{X9} and R^{X10} applied to the structure of G represents any combination described in Combination B (hereinafter, referred to as Compound group SX728).

A compound according to Compound (2B) wherein R¹ represents a chlorine atom, Q represents Q6, and a combination of structure of G and substituents R^{X7}, R^{X8}, R^{X9} and R^{X10} applied to the structure of G represents any combination described in Combination B (hereinafter, referred to as Compound group SX729).

A compound according to Compound (2B) wherein R¹ represents a methyl group, Q represents Q7, L² represents an oxygen atom, and a combination of structure of G and substituents R^{X7}, R^{X8}, R^{X9} and R^{X10} applied to the structure of G represents any combination described in Combination B (hereinafter, referred to as Compound group SX730).

A compound according to Compound (2B) wherein R¹ represents a chlorine atom, Q represents Q7, L² represents an oxygen atom, and a combination of structure of G and substituents R^{X7}, R^{X8}, R^{X9} and R^{X10} applied to the structure of G represents any combination described in Combination B (hereinafter, referred to as Compound group SX731).

A compound according to Compound (2B) wherein R¹ represents a methyl group, Q represents Q7, L² represents NH, and a combination of structure of G and substituents R^{X7}, R^{X8}, R^{X9} and R^{X10} applied to the structure of G represents any combination described in Combination B (hereinafter, referred to as Compound group SX732).

A compound according to Compound (2B) wherein R¹ represents a chlorine atom, Q represents Q7, L² represents NH, and a combination of structure of G and substituents R^{X7}, R^{X8}, R^{X9} and R^{X10} applied to the structure of G represents any combination described in Combination B (hereinafter, referred to as Compound group SX733).

A compound according to Compound (2B) wherein R¹ represents a methyl group, Q represents Q8A, and a combination of structure of G and substituents R^{X7}, R^{X8}, R^{X9} and R^{X10} applied to the structure of G represents any combination described in Combination B (hereinafter, referred to as Compound group SX734).

A compound according to Compound (2B) wherein R¹ represents a chlorine atom, Q represents Q8A, and a combination of structure of G and substituents R^{X7}, R^{X8}, R^{X9} and R^{X10} applied to the structure of G represents any combination described in Combination B (hereinafter, referred to as Compound group SX735).

A compound according to Compound (2B) wherein R¹ represents a methyl group, Q represents Q8D, and a combination of structure of G and substituents R^{X7}, Rx, R^{X9} and R^{X10} applied to the structure of G represents any combination described in Combination B (hereinafter, referred to as Compound group SX736).

A compound according to Compound (2B) wherein R¹ represents a chlorine atom, Q represents Q8D, and a combination of structure of G and substituents R^{X7}, Rx, R^{X9} and R^{X10} applied to the structure of G represents any combination described in Combination B (hereinafter, referred to as Compound group SX737).

A compound according to Compound (2B) wherein R¹ represents a methyl group, Q represents Q8J, and a combination of structure of G and substituents R^{X7}, Rx, R^{X9} and R^{X10} applied to the structure of G represents any combination described in Combination B (hereinafter, referred to as Compound group SX738).

A compound according to Compound (2B) wherein R¹ represents a chlorine atom, Q represents Q8J, and a combination of structure of G and substituents R^{X7}, Rx, R^{X9} and R^{X10} applied to the structure of G represents any combination described in Combination B (hereinafter, referred to as Compound group SX739).

A compound represented by formula (2D) (hereinafter, referred to as Compound (2D)):

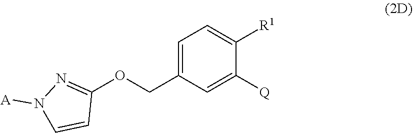

[wherein A represents any one of groups represented by formulae AA1 to AA4.]
wherein R¹ represents a methyl group, Q represents Q1, and a combination of structure of A and substituents R^{X11}, R^{X12}, R^{X13} and R^{X14} applied to the structure of A represents any combination described in Combination D (hereinafter, referred to as Compound group SX740).

A compound according to Compound (2D) wherein R¹ represents a chlorine atom, Q represents Q1, and a combination of structure of A and substituents R^{X11}, R^{X12}, R^{X13} and R^{X14} applied to the structure of A represents any combination described in Combination D (hereinafter, referred to as Compound group SX741).

A compound according to Compound (2D) wherein R¹ represents an ethyl group, Q represents Q1, and a combination of structure of A and substituents R^{X11}, R^{X12}, R^{X13} and R^{X14} applied to the structure of A represents any combination described in Combination D (hereinafter, referred to as Compound group SX742).

A compound according to Compound (2D) wherein R¹ represents a fluorine atom, Q represents Q1, and a combination of structure of A and substituents R^{X11}, R^{X12}, R^{X13} and R^{X14} applied to the structure of A represents any combination described in Combination D (hereinafter, referred to as Compound group SX743).

A compound according to Compound (2D) wherein R¹ represents a cyclopropyl group, Q represents Q1, and a combination of structure of A and substituents R^{X11}, R^{X12}, R^{X13} and R^{X14} applied to the structure of A represents any combination described in Combination D (hereinafter, referred to as Compound group SX744).

A compound according to Compound (2D) wherein R¹ represents a methyl group, Q represents Q2, L¹ represents an oxygen atom, and a combination of structure of A and substituents R^{X11}, R^{X12}, R^{X13} and R^{X14} applied to the structure of A represents any combination described in Combination D (hereinafter, referred to as Compound group SX745).

A compound according to Compound (2D) wherein R¹ represents a chlorine atom, Q represents Q2, L¹ represents an oxygen atom, and a combination of structure of A and substituents R^{X11}, R^{X12}, R^{X13} and R^{X14} applied to the structure of A represents any combination described in Combination D (hereinafter, referred to as Compound group SX746).

A compound according to Compound (2D) wherein R¹ represents a methyl group, Q represents Q2, L¹ represents NH, and a combination of structure of A and substituents R^{X11}, R^{X12}, R^{X13} and R^{X14} applied to the structure of A represents any combination described in Combination D (hereinafter, referred to as Compound group SX747).

A compound according to Compound (2D) wherein R¹ represents a chlorine atom, Q represents Q2, L¹ represents NH, and a combination of structure of A and substituents R^{X11}, R^{X12}, R^{X13} and R^{X14} applied to the structure of A represents any combination described in Combination D (hereinafter, referred to as Compound group SX748).

A compound according to Compound (2D) wherein R¹ represents a methyl group, Q represents Q3, R³ represents a methoxy group, and a combination of structure of A and substituents R^{X11}, R^{X12}, R^{X13} and R^{X14} applied to the structure of A represents any combination described in Combination D (hereinafter, referred to as Compound group SX749).

A compound according to Compound (2D) wherein R¹ represents a chlorine atom, Q represents Q3, R³ represents a methoxy group, and a combination of structure of A and substituents R^{X11}, R^{X12}, R^{X13} and R^{X14} applied to the structure of A represents any combination described in Combination D (hereinafter, referred to as Compound group SX750).

A compound according to Compound (2D) wherein R¹ represents a methyl group, Q represents Q4, and a combination of structure of A and substituents R^{X11}, R^{X12}, R^{X13} and R^{X14} applied to the structure of A represents any combination described in Combination D (hereinafter, referred to as Compound group SX751).

A compound according to Compound (2D) wherein R¹ represents a chlorine atom, Q represents Q4, and a combination of structure of A and substituents R^{X11}, R^{X12}, R^{X13} and R^{X14} applied to the structure of A represents any combination described in Combination D (hereinafter, referred to as Compound group SX752).

A compound according to Compound (2D) wherein R¹ represents a methyl group, Q represents Q5, and a combination of structure of A and substituents R^{X11}, R^{X12}, R^{X13} and R^{X14} applied to the structure of A represents any combination described in Combination D (hereinafter, referred to as Compound group SX753).

A compound according to Compound (2D) wherein R¹ represents a chlorine atom, Q represents Q5, and a combination of structure of A and substituents R^{X11}, R^{X12}, R^{X13} and R^{X14} applied to the structure of A represents any combination described in Combination D (hereinafter, referred to as Compound group SX754).

A compound according to Compound (2D) wherein R¹ represents a methyl group, Q represents Q6, and a combination of structure of A and substituents R^{X11}, R^{X12}, R^{X13} and R^{X14} applied to the structure of A represents any combination described in Combination D (hereinafter, referred to as Compound group SX755).

A compound according to Compound (2D) wherein R¹ represents a chlorine atom, Q represents Q6, and a combination of structure of A and substituents R^{X11}, R^{X12}, R^{X13} and R^{X14} applied to the structure of A represents any combination described in Combination D (hereinafter, referred to as Compound group SX756).

A compound according to Compound (2D) wherein R¹ represents a methyl group, Q represents Q7, L² represents an oxygen atom, and a combination of structure of A and substituents R^{X11}, R^{X12}, R^{X13} and R^{X14} applied to the structure of A represents any combination described in Combination D (hereinafter, referred to as Compound group SX757).

A compound according to Compound (2D) wherein R¹ represents a chlorine atom, Q represents Q7, L² represents an oxygen atom, and a combination of structure of A and substituents R^{X11}, R^{X12}, R^{X13} and R^{X14} applied to the structure of A represents any combination described in Combination D (hereinafter, referred to as Compound group SX758).

A compound according to Compound (2D) wherein R¹ represents a methyl group, Q represents Q7, L² represents NH, and a combination of structure of A and substituents R^{X11}, R^{X12}, R^{X13} and R^{X14} applied to the structure of A represents any combination described in Combination D (hereinafter, referred to as Compound group SX759).

A compound according to Compound (2D) wherein R¹ represents a chlorine atom, Q represents Q7, L² represents NH, and a combination of structure of A and substituents R^{X11}, R^{X12}, R^{X13} and R^{X14} applied to the structure of A represents any combination described in Combination D (hereinafter, referred to as Compound group SX760).

A compound according to Compound (2D) wherein R¹ represents a methyl group, Q represents Q8A, and a combination of structure of A and substituents R^{X11}, R^{X12}, R^{X13} and R^{X14} applied to the structure of A represents any combination described in Combination D (hereinafter, referred to as Compound group SX761).

A compound according to Compound (2D) wherein R¹ represents a chlorine atom, Q represents Q8A, and a combination of structure of A and substituents R^{X11}, R^{X12}, R^{X13} and R^{X14} applied to the structure of A represents any combination described in Combination D (hereinafter, referred to as Compound group SX762).

A compound according to Compound (2D) wherein R¹ represents a methyl group, Q represents Q8D, and a combination of structure of A and substituents R^{X11}, R^{X12}, R^{X13} and R^{X14} applied to the structure of A represents any combination described in Combination D (hereinafter, referred to as Compound group SX763).

A compound according to Compound (2D) wherein R¹ represents a chlorine atom, Q represents Q8D, and a combination of structure of A and substituents R^{X11}, R^{X12}, R^{X13} and R^{X14} applied to the structure of A represents any combination described in Combination D (hereinafter, referred to as Compound group SX764).

A compound according to Compound (2D) wherein R¹ represents a methyl group, Q represents Q8J, and a combination of structure of A and substituents R^{X11}, R^{X12}, R^{X13} and R^{X14} applied to the structure of A represents any combination described in Combination D (hereinafter, referred to as Compound group SX765).

A compound according to Compound (2D) wherein R¹ represents a chlorine atom, Q represents Q8J, and a combination of structure of A and substituents R^{X11}, R^{X12}, R^{X13} and R^{X14} applied to the structure of A represents any combination described in Combination D (hereinafter, referred to as Compound group SX766).

A compound represented by formula (2E) (hereinafter, referred to as Compound (2E)):

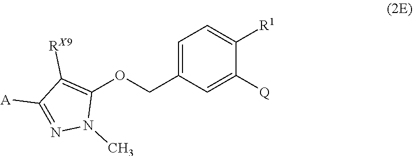

[wherein A represents any one of groups represented by formulae AA1 to AA4.]
wherein R¹ represents a methyl group, R^{X9} represents a methyl group, Q represents Q1, and a combination of structure of A and substituents R^{X11}, R^{X12}, R^{X13} and R^{X14} applied to the structure of A represents any combination described in Combination D (hereinafter, referred to as Compound group SX767).

A compound according to Compound (2E) wherein R¹ represents a chlorine atom, R^{X9} represents a methyl group, Q represents Q1, and a combination of structure of A and substituents R^{X11}, R^{X12}, R^{X13} and R^{X14} applied to the structure of A represents any combination described in Combination D (hereinafter, referred to as Compound group SX768).

A compound according to Compound (2E) wherein R¹ represents a methyl group, R^{X9} represents a methyl group, Q represents Q2, L¹ represents an oxygen atom, and a combination of structure of A and substituents R^{X11}, R^{X12}, R^{X13} and R^{X14} applied to the structure of A represents any combination described in Combination D (hereinafter, referred to as Compound group SX769).

A compound according to Compound (2E) wherein R¹ represents a chlorine atom, R^{X9} represents a methyl group, Q represents Q2, L¹ represents an oxygen atom, and a combination of structure of A and substituents R^{X11}, R^{X12}, R^{X13} and R^{X14} applied to the structure of A represents any combination described in Combination D (hereinafter, referred to as Compound group SX770).

A compound according to Compound (2E) wherein R¹ represents a methyl group, R^{X9} represents a methyl group, Q represents Q2, L¹ represents NH, and a combination of structure of A and substituents R^{X11}, R^{X12}, R^{X13} and R^{X14} applied to the structure of A represents any combination described in Combination D (hereinafter, referred to as Compound group SX771).

A compound according to Compound (2E) wherein R¹ represents a chlorine atom, R^{X9} represents a methyl group, Q represents Q2, L¹ represents NH, and a combination of structure of A and substituents R^{X11}, R^{X12}, R^{X13} and R^{X14} applied to the structure of A represents any combination described in Combination D (hereinafter, referred to as Compound group SX772).

A compound according to Compound (2E) wherein R¹ represents a methyl group, R^{X9} represents a methyl group, Q represents Q3, R³ represents a methoxy group, and a combination of structure of A and substituents R^{X11}, R^{X12}, R^{X13} and R^{X14} applied to the structure of A represents any combination described in Combination D (hereinafter, referred to as Compound group SX773).

A compound according to Compound (2E) wherein R¹ represents a chlorine atom, R^{X9} represents a methyl group, Q represents Q3, R³ represents a methoxy group, and a combination of structure of A and substituents R^{X11}, R^{X12}, R^{X13} and R^{X14} applied to the structure of A represents any combination described in Combination D (hereinafter, referred to as Compound group SX774).

A compound according to Compound (2E) wherein R¹ represents a methyl group, R^{X9} represents a methyl group, Q represents Q4, and a combination of structure of A and substituents R^{X11}, R^{X12}, R^{X13} and R^{X14} applied to the structure of A represents any combination described in Combination D (hereinafter, referred to as Compound group SX775).

A compound according to Compound (2E) wherein R¹ represents a chlorine atom, R^{X9} represents a methyl group, Q represents Q4, and a combination of structure of A and substituents R^{X11}, R^{X12}, R^{X13} and R^{X14} applied to the structure of A represents any combination described in Combination D (hereinafter, referred to as Compound group SX776).

A compound according to Compound (2E) wherein R¹ represents a methyl group, R^{X9} represents a methyl group, Q represents Q5, and a combination of structure of A and substituents R^{X11}, R^{X12}, R^{X13} and R^{X14} applied to the structure of A represents any combination described in Combination D (hereinafter, referred to as Compound group SX777).

A compound according to Compound (2E) wherein R¹ represents a chlorine atom, R^{X9} represents a methyl group, Q represents Q5, and a combination of structure of A and substituents R^{X11}, R^{X12}, R^{X13} and R^{X14} applied to the structure of A represents any combination described in Combination D (hereinafter, referred to as Compound group SX778).

A compound according to Compound (2E) wherein R¹ represents a methyl group, R^{X9} represents a methyl group, Q represents Q6, and a combination of structure of A and substituents R^{X11}, R^{X12}, R^{X13} and R^{X14} applied to the structure of A represents any combination described in Combination D (hereinafter, referred to as Compound group SX779).

A compound according to Compound (2E) wherein R¹ represents a chlorine atom, R^{X9} represents a methyl group, Q represents Q6, and a combination of structure of A and substituents R^{X11}, R^{X12}, R^{X13} and R^{X14} applied to the structure of A represents any combination described in Combination D (hereinafter, referred to as Compound group SX780).

A compound according to Compound (2E) wherein R¹ represents a methyl group, R^{X9} represents a methyl group, Q represents Q7, L² represents an oxygen atom, and a combination of structure of A and substituents R^{X11}, R^{X12}, R^{X13} and R^{X14} applied to the structure of A represents any combination described in Combination D (hereinafter, referred to as Compound group SX781).

A compound according to Compound (2E) wherein R¹ represents a chlorine atom, R^{X9} represents a methyl group, Q represents Q7, L² represents an oxygen atom, and a combination of structure of A and substituents R^{X11}, R^{X12}, R^{X13} and R^{X14} applied to the structure of A represents any combination described in Combination D (hereinafter, referred to as Compound group SX782).

A compound according to Compound (2E) wherein R¹ represents a methyl group, R^{X9} represents a methyl group, Q represents Q7, L² represents NH, and a combination of structure of A and substituents R^{X11}, R^{X12}, R^{X13} and R^{X14} applied to the structure of A represents any combination described in Combination D (hereinafter, referred to as Compound group SX783).

A compound according to Compound (2E) wherein R¹ represents a chlorine atom, R^{X9} represents a methyl group, Q represents Q7, L² represents NH, and a combination of structure of A and substituents R^{X11}, R^{X12}, R^{X13} and R^{X14} applied to the structure of A represents any combination described in Combination D (hereinafter, referred to as Compound group SX784).

A compound according to Compound (2E) wherein R¹ represents a methyl group, R^{X9} represents a methyl group, Q represents Q8A, and a combination of structure of A and substituents R^{X11}, R^{X12}, R^{X13} and R^{X14} applied to the structure of A represents any combination described in Combination D (hereinafter, referred to as Compound group SX785).

A compound according to Compound (2E) wherein R¹ represents a chlorine atom, R^{X9} represents a methyl group, Q represents Q8A, and a combination of structure of A and substituents R^{X11}, R^{X12}, R^{X13} and R^{X14} applied to the structure of A represents any combination described in Combination D (hereinafter, referred to as Compound group SX786).

A compound according to Compound (2E) wherein R¹ represents a methyl group, R^{X9} represents a methyl group, Q represents Q8D, and a combination of structure of A and substituents R^{X11}, R^{X12}, R^{X13} and R^{X14} applied to the structure of A represents any combination described in Combination D (hereinafter, referred to as Compound group SX787).

A compound according to Compound (2E) wherein R¹ represents a chlorine atom, R^{X9} represents a methyl group, Q represents Q8D, and a combination of structure of A and substituents R^{X11}, R^{X12}, R^{X13} and R^{X14} applied to the structure of A represents any combination described in Combination D (hereinafter, referred to as Compound group SX788).

A compound according to Compound (2E) wherein R¹ represents a methyl group, R^{X9} represents a methyl group, Q represents Q8J, and a combination of structure of A and substituents R^{X11}, R^{X12}, R^{X13} and R^{X14} applied to the structure of A represents any combination described in Combination D (hereinafter, referred to as Compound group SX789).

A compound according to Compound (2E) wherein R¹ represents a chlorine atom, R^{X9} represents a methyl group, Q represents Q8J, and a combination of structure of A and substituents R^{X11}, R^{X12}, R^{X13} and R^{X14} applied to the structure of A represents any combination described in Combination D (hereinafter, referred to as Compound group SX790).

A compound according to Compound (2E) wherein R¹ represents a methyl group, R^{X9} represents a hydrogen atom, Q represents Q1, and a combination of structure of A and substituents R^{X11}, R^{X12}, R^{X13} and R^{X14} applied to the structure of A represents any combination described in Combination D (hereinafter, referred to as Compound group SX791).

A compound according to Compound (2E) wherein R¹ represents a chlorine atom, R^{X9} represents a hydrogen atom, Q represents Q1, and a combination of structure of A and substituents R^{X11}, R^{X12}, R^{X13} and R^{X14} applied to the structure of A represents any combination described in Combination D (hereinafter, referred to as Compound group SX792).

A compound according to Compound (2E) wherein R¹ represents a methyl group, R^{X9} represents a hydrogen atom, Q represents Q2, L¹ represents an oxygen atom, and a combination of structure of A and substituents R^{X11}, R^{X12}, R^{X13} and R^{X14} applied to the structure of A represents any combination described in Combination D (hereinafter, referred to as Compound group SX793).

A compound according to Compound (2E) wherein R¹ represents a chlorine atom, R^{X9} represents a hydrogen atom, Q represents Q2, L¹ represents an oxygen atom, and a combination of structure of A and substituents R^{X11}, R^{X2}, R^{X13} and R^{X14} applied to the structure of A represents any combination described in Combination D (hereinafter, referred to as Compound group SX794).

A compound according to Compound (2E) wherein R¹ represents a methyl group, R^{X9} represents a hydrogen atom, Q represents Q2, L¹ represents NH, and a combination of structure of A and substituents R^{X11}, R^{X2}, R^{X13} and R^{X14} applied to the structure of A represents any combination described in Combination D (hereinafter, referred to as Compound group SX795).

A compound according to Compound (2E) wherein R¹ represents a chlorine atom, R^{X9} represents a hydrogen atom, Q represents Q2, L¹ represents NH, and a combination of structure of A and substituents R^{X11}, R^{X2}, R^{X13} and R^{X14} applied to the structure of A represents any combination described in Combination D (hereinafter, referred to as Compound group SX796).

A compound according to Compound (2E) wherein R¹ represents a methyl group, R^{X9} represents a hydrogen atom, Q represents Q3, R³ represents a methoxy group, and a combination of structure of A and substituents R^{X11}, R^{X2}, R^{X13} and R^{X14} applied to the structure of A represents any combination described in Combination D (hereinafter, referred to as Compound group SX797).

A compound according to Compound (2E) wherein R¹ represents a chlorine atom, R^{X9} represents a hydrogen atom, Q represents Q3, R³ represents a methoxy group, and a combination of structure of A and substituents R^{X11}, R^{X12}, R^{X13} and R^{X14} applied to the structure of A represents any combination described in Combination D (hereinafter, referred to as Compound group SX798).

A compound according to Compound (2E) wherein R¹ represents a methyl group, R^{X9} represents a hydrogen atom, Q represents Q4, and a combination of structure of A and substituents R^{X11}, R^{X12}, R^{X13} and R^{X14} applied to the structure of A represents any combination described in Combination D (hereinafter, referred to as Compound group SX799).

A compound according to Compound (2E) wherein R¹ represents a chlorine atom, R^{X9} represents a hydrogen atom, Q represents Q4, and a combination of structure of A and substituents R^{X11}, R^{X12}, R^{X13} and R^{X14} applied to the structure of A represents any combination described in Combination D (hereinafter, referred to as Compound group SX800).

A compound according to Compound (2E) wherein R¹ represents a methyl group, R^{X9} represents a hydrogen atom, Q represents Q5, and a combination of structure of A and substituents R^{X11}, R^{X12}. R^{X13} and R^{X14} applied to the structure of A represents any combination described in Combination D (hereinafter, referred to as Compound group SX801).

A compound according to Compound (2E) wherein R¹ represents a chlorine atom, R^{X9} represents a hydrogen atom, Q represents Q5, and a combination of structure of A and substituents R^{X11}, R^{X12}, R^{X13} and R^{X14} applied to the structure of A represents any combination described in Combination D (hereinafter, referred to as Compound group SX802).

A compound according to Compound (2E) wherein R¹ represents a methyl group, R^{X9} represents a hydrogen atom, Q represents Q6, and a combination of structure of A and substituents R^{X11}, R^{X12}, R^{X13} and R^{X14} applied to the structure of A represents any combination described in Combination D (hereinafter, referred to as Compound group SX803).

A compound according to Compound (2E) wherein R¹ represents a chlorine atom, R^{X9} represents a hydrogen atom, Q represents Q6, and a combination of structure of A and substituents R^{X11}, R^{X12}, R^{X13} and R^{X14} applied to the structure of A represents any combination described in Combination D (hereinafter, referred to as Compound group SX804).

A compound according to Compound (2E) wherein R¹ represents a methyl group, R^{X9} represents a hydrogen atom, Q represents Q7, L² represents an oxygen atom, and a combination of structure of A and substituents R^{X11}, R^{X12}, R^{X13} and R^{X14} applied to the structure of A represents any combination described in Combination D (hereinafter, referred to as Compound group SX805).

A compound according to Compound (2E) wherein R¹ represents a chlorine atom, R^{X9} represents a hydrogen atom, Q represents Q7, L² represents an oxygen atom, and a combination of structure of A and substituents R^{X11}, R^{X12}, R^{X13} and R^{X14} applied to the structure of A represents any combination described in Combination D (hereinafter, referred to as Compound group SX806).

A compound according to Compound (2E) wherein R¹ represents a methyl group, R^{X9} represents a hydrogen atom, Q represents Q7, L² represents NH, and a combination of structure of A and substituents R^{X11}, R^{X12}, R^{X13} and R^{X14} applied to the structure of A represents any combination described in Combination D (hereinafter, referred to as Compound group SX807).

A compound according to Compound (2E) wherein R¹ represents a chlorine atom, R^{X9} represents a hydrogen atom, Q represents Q7, L² represents NH, and a combination of structure of A and substituents R^{X11}, R^{X12}, R^{X13} and R^{X14} applied to the structure of A represents any combination described in Combination D (hereinafter, referred to as Compound group SX808).

A compound according to Compound (2E) wherein R¹ represents a methyl group, R^{X9} represents a hydrogen atom, Q represents Q8A, and a combination of structure of A and substituents R^{X11}, R^{X12}, R^{X13} and R^{X14} applied to the structure of A represents any combination described in Combination D (hereinafter, referred to as Compound group SX809).

A compound according to Compound (2E) wherein R¹ represents a chlorine atom, R^{X9} represents a hydrogen atom, Q represents Q8A, and a combination of structure of A and substituents R^{X11}, R^{X12}, R^{X13} and R^{X14} applied to the structure of A represents any combination described in Combination D (hereinafter, referred to as Compound group SX810).

A compound according to Compound (2E) wherein R¹ represents a methyl group, R^{X9} represents a hydrogen atom, Q represents Q8D, and a combination of structure of A and substituents R^{X11}, R^{X12}, R^{X13} and R^{X14} applied to the structure of A represents any combination described in Combination D (hereinafter, referred to as Compound group SX811).

A compound according to Compound (2E) wherein R¹ represents a chlorine atom, R^{X9} represents a hydrogen atom, Q represents Q8D, and a combination of structure of A and substituents R^{X11}, R^{X12}, R^{X13} and R^{X14} applied to the structure of A represents any combination described in Combination D (hereinafter, referred to as Compound group SX812).

A compound according to Compound (2E) wherein R¹ represents a methyl group, R^{X9} represents a hydrogen atom, Q represents Q8J, and a combination of structure of A and substituents R^{X11}, R^{X12}, R^{X13} and R^{X14} applied to the structure of A represents any combination described in Combination D (hereinafter, referred to as Compound group SX813).

A compound according to Compound (2E) wherein R¹ represents a chlorine atom, R^{X9} represents a hydrogen atom, Q represents Q8J, and a combination of structure of A and substituents R^{X11}, R^{X12}, R^{X13} and R^{X14} applied to the structure of A represents any combination described in Combination D (hereinafter, referred to as Compound group SX814).

A compound represented by formula (2F) (hereinafter, referred to as Compound (2F)):

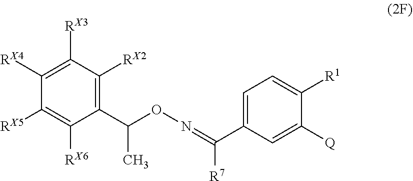

wherein R¹ represents a methyl group, Q represents Q1, R⁷ represents a hydrogen atom, and a combination of R^{X2}, R^{X3}, R^{X4}, R^{X5} and R^{X6} represents any combination described in Combination A (hereinafter, referred to as Compound group SX815).

A compound according to Compound (2F) wherein R¹ represents a chlorine atom, Q represents Q1, R⁷ represents a hydrogen atom, and a combination of R^{X2}, R^{X3}, R^{X4}, R^{X5} and R^{X6} represents any combination described in Combination A (hereinafter, (hereinafter, referred to as Compound group SX816).

A compound according to Compound (2F) wherein R¹ represents a methyl group, Q represents Q2, L¹ represents an oxygen atom, R⁷ represents a hydrogen atom, and a combination of R^{X2}, R^{X3}, R^{X4}, R^{X5} and R^{X6} represents any combination described in Combination A (hereinafter, referred to as Compound group SX817).

A compound according to Compound (2F) wherein R¹ represents a chlorine atom, Q represents Q2, L¹ represents an oxygen atom, R⁷ represents a hydrogen atom, and a combination of R^{X2}, R^{X3}, R^{X4}, R^{X5} and R^{X6} represents any combination described in Combination A (hereinafter, referred to as Compound group SX818).

A compound according to Compound (2F) wherein R¹ represents a methyl group, Q represents Q2, L¹ represents NH, R⁷ represents a hydrogen atom, and a combination of R^{X2}, R^{X3}, R^{X4}, R^{X5} and R^{X6} represents any combination described in Combination A (hereinafter, referred to as Compound group SX819).

A compound according to Compound (2F) wherein R¹ represents a chlorine atom, Q represents Q2, L¹ represents NH, R⁷ represents a hydrogen atom, and a combination of R^{X2}, R^{X3}, R^{X4}, R^{X5} and R^{X6} represents any combination described in Combination A (hereinafter, referred to as Compound group SX820).

A compound according to Compound (2F) wherein R¹ represents a methyl group, Q represents Q3, R³ represents a methoxy group, R⁷ represents a hydrogen atom, and a combination of R^{X2}, R^{X3}, R^{X4}, R^{X5} and R^{X6} represents any combination described in Combination A (hereinafter, referred to as Compound group SX821).

A compound according to Compound (2F) wherein R¹ represents a chlorine atom, Q represents Q3, R³ represents a methoxy group, R⁷ represents a hydrogen atom, and a combination of R^{X2}, R^{X3}, R^{X4}, R^{X5} and R^{X6} represents any combination described in Combination A (hereinafter, referred to as Compound group SX822).

A compound according to Compound (2F) wherein R¹ represents a methyl group, Q represents Q4, R⁷ represents a hydrogen atom, and a combination of R^{X2}, R^{X3}, R^{X4}, R^{X5} and R^{X6} represents any combination described in Combination A (hereinafter, referred to as Compound group SX823).

A compound according to Compound (2F) wherein R¹ represents a chlorine atom, Q represents Q4, R⁷ represents a hydrogen atom, and a combination of R^{X2}, R^{X3}, R^{X4}, R^{X5} and R^{X6} represents any combination described in Combination A (hereinafter, referred to as Compound group SX824).

A compound according to Compound (2F) wherein R¹ represents a methyl group, Q represents Q5, R⁷ represents a hydrogen atom, and a combination of R^{X2}, R^{X3}, R^{X4}, R^{X5} and R^{X6} represents any combination described in Combination A (hereinafter, referred to as Compound group SX825).

A compound according to Compound (2F) wherein R¹ represents a chlorine atom, Q represents Q5, R⁷ represents a hydrogen atom, and a combination of R^{X2}, R^{X3}, R^{X4}, R^{X5} and R^{X6} represents any combination described in Combination A (hereinafter, referred to as Compound group SX826).

A compound according to Compound (2F) wherein R¹ represents a methyl group, Q represents Q6, R⁷ represents a hydrogen atom, and a combination of R^{X2}, R^{X3}, R^{X4}, R^{X5} and R^{X6} represents any combination described in Combination A (hereinafter, referred to as Compound group SX827).

A compound according to Compound (2F) wherein R¹ represents a chlorine atom, Q represents Q6, R⁷ represents a hydrogen atom, and a combination of R^{X2}, R^{X3}, R^{X4}, R^{X5} and R^{X6} represents any combination described in Combination A (hereinafter, referred to as Compound group SX828).

A compound according to Compound (2F) wherein R¹ represents a methyl group, Q represents Q7, L² represents an oxygen atom, R⁷ represents a hydrogen atom, and a combination of R^{X2}, R^{X3}, R^{X4}, R^{X5} and R^{X6} represents any combination described in Combination A (hereinafter, referred to as Compound group SX829).

A compound according to Compound (2F) wherein R¹ represents a chlorine atom, Q represents Q7, L² represents an oxygen atom, R⁷ represents a hydrogen atom, and a combination of R^{X2}, R^{X3}, R^{X4}, R^{X5} and R^{X6} represents any combination described in Combination A (hereinafter, referred to as Compound group SX830).

A compound according to Compound (2F) wherein R¹ represents a methyl group, Q represents Q7, L² represents NH, R⁷ represents a hydrogen atom, and a combination of R^{X2}, R^{X3}, R^{X4}, R^{X5} and R^{X6} represents any combination described in Combination A (hereinafter, referred to as Compound group SX831).

A compound according to Compound (2F) wherein R¹ represents a chlorine atom, Q represents Q7, L² represents NH, R⁷ represents a hydrogen atom, and a combination of R^{X2}, R^{X3}, R^{X4}, R^{X5} and R^{X6} represents any combination described in Combination A (hereinafter, referred to as Compound group SX832).

A compound according to Compound (2F) wherein R¹ represents a methyl group, Q represents Q8A, R⁷ represents a hydrogen atom, and a combination of R^{X2}, R^{X3}, R^{X4}, R^{X5} and R^{X6} represents any combination described in Combination A (hereinafter, referred to as Compound group SX833).

A compound according to Compound (2F) wherein R¹ represents a chlorine atom, Q represents Q8A, R⁷ represents a hydrogen atom, and a combination of R^{X2}, R^{X3}, R^{X4}, R^{X5} and R^{X6} represents any combination described in Combination A (hereinafter, referred to as Compound group SX834).

A compound according to Compound (2F) wherein R¹ represents a methyl group, Q represents Q8D, R⁷ represents a hydrogen atom, and a combination of R^{X2}, R^{X3}, R^{X4}, R^{X5} and R^{X6} represents any combination described in Combination A (hereinafter, referred to as Compound group SX835).

A compound according to Compound (2F) wherein R¹ represents a chlorine atom, Q represents Q8D, R⁷ represents a hydrogen atom, and a combination of R^{X2}, R^{X3}, R^{X4}, R^{X5} and R^{X6} represents any combination described in Combination A (hereinafter, referred to as Compound group SX836).

A compound according to Compound (2F) wherein R¹ represents a methyl group, Q represents Q8J, R⁷ represents a hydrogen atom, and a combination of R^{X2}, R^{X3}, R^{X4}, R^{X5} and R^{X6} represents any combination described in Combination A (hereinafter, referred to as Compound group SX837).

A compound according to Compound (2F) wherein R¹ represents a chlorine atom, Q represents Q8J, R⁷ represents a hydrogen atom, and a combination of R^{X2}, R^{X3}, R^{X4}, R^{X5} and R^{X6} represents any combination described in Combination A (hereinafter, referred to as Compound group SX838).

A compound represented by formula (2G) (hereinafter, referred to as Compound (2G)):

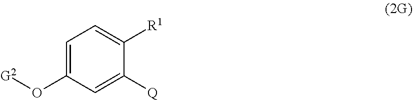

[wherein G² represents any one of formula G1, formula G9, formula G10 and formula G22.]
wherein R¹ represents a methyl group, Q represents Q1, and a combination of structure of G² and substituents R^{X7}, R^{X8}, R^{X9} and R^{X10} applied to the structure of G² represents any combination described in Combination J (hereinafter, referred to as Compound group SX839).

Combination J consists of substituent numbers ZJ1 to ZJ50. Substituent numbers ZJ1 to ZJ50 represent combinations of structure of G² and substituents of R^{X7}, R^{X8}, R^{X9} and R^{X10} applied to the structure of G² according to Compound (2G). And hereinafter, they are referred to as [substituent number; G², R^{X7}, R^{X8}, R^{X9}, R^{X10}]. For example, the substituent number ZJ2 refers to a combination wherein G² represents G1, R^{X8} represents a methyl group, and each of R^{X7}, R^{X9} and R^{X10} represents a hydrogen atom.
Combination J: [ZJ1;G1,Me,H,H,H], [ZJ2;G1,H,Me,H,H], [ZJ3;G1,H,H,Me,H], [ZJ4; G1,CF₃,H,H,H], [ZJ5;G1,H,CF₃,H,H], [ZJ6;G1,H,H,CF₃,H], [ZJ7;G1, Me,H,Me,H], [ZJ8;G1,Me,H,CF₃,H], [ZJ9;G9,H,Me,H, -], [ZJ10;G9,H,H,Me,-], [ZJ11;G9,H,Me,Me,-], [ZJ12;G9,H,t-Bu,H,-], [ZJ13;G9,H,H,t-Bu,-], [ZJ14;G9,CF₃,H,H,-], [ZJ15;G9,C1,H,H,-], [ZJ16;G9,H,Cl, H,-], [ZJ17;G9,H,H,Cl,-], [ZJ18;G9,H,Cl,Me,-], [ZJ19;G9,H,Br,H,-], [ZJ20;G9,H,H,Br,-], [ZJ21;G9,H,Br,Me,-], [ZJ22;G9,H,I,H,-], [ZJ23;G9,H,H,I,-], [ZJ24;G9,H,I,Me,-], [ZJ25;G10,H,Me,H,-], [ZJ26;G10,H,H,Me,-], [ZJ27;G10,H,Me,Me,-], [ZJ28;G10,H,t-Bu,H,-], [ZJ29;G10,H,H,t-Bu,-], [ZJ30;G10,Cl,H,H,-], [ZJ31;G10,H,Cl,H,-], [ZJ32;G10,H,H,Cl,-], [ZJ33;G10,H,Cl,Me,-], [ZJ34;G10,H,Br,H,-], [ZJ35;G10,H,H,Br,-], [ZJ36;G10,H,Br,Me,-], [ZJ37;G10,H,I,H,-], [ZJ38;G10, H,H,I,-], [ZJ39;G10,H,I,Me, -], [ZJ40;G22,CF₃,H, -,-], [ZJ41;G22,OCF₃,H,-,-], [ZJ42;G22,SMe,H,-,-], [ZJ43;G22,Cl,H, -,-], [ZJ44;G22,I,H,-, -], [ZJ45;G22,CCN,H, -,-], [ZJ46;G22,SiMe₃,H,-, -], [Z J47;G22,CF₃,Me,-,-], [ZJ48;G22,Cl,Me,-,-], [ZJ49;G22,I,Me,-,-], [ZJ50;G22,CN,Me,-,-]

A compound according to Compound (2G) wherein R¹ represents a chlorine atom, Q represents Q1, and a combination of structure of G² and substituents R^{X7}, R^{X8}, R^{X9} and R^{X10} applied to the structure of G² represents any combination described in Combination J (hereinafter, referred to as Compound group SX840).

A compound according to Compound (2G) wherein R¹ represents a methyl group, Q represents Q2, L represents an oxygen atom, and a combination of structure of G² and substituents R^{X7}, R^{X8}, R^{X9} and R^{X10} applied to the structure of G² represents any combination described in Combination J (hereinafter, referred to as Compound group SX841).

A compound according to Compound (2G) wherein R¹ represents a chlorine atom, Q represents Q2, L represents an oxygen atom, and a combination of structure of G² and substituents R^{X7}, R^{X8}, R^{X9} and R^{X10} applied to the structure of G² represents any combination described in Combination J (hereinafter, referred to as Compound group SX842).

A compound according to Compound (2G) wherein R¹ represents a methyl group, Q represents Q2, L represents NH, and a combination of structure of G² and substituents R^{X7}, R^{X8}, R^{X9} and R^{X10} applied to the structure of G² represents any combination described in Combination J (hereinafter, referred to as Compound group SX843).

A compound according to Compound (2G) wherein R¹ represents a chlorine atom, Q represents Q2, L represents NH, and a combination of structure of G² and substituents R^{X7}, R^{X8}, R^{X9} and R^{X10} applied to the structure of G² represents any combination described in Combination J (hereinafter, referred to as Compound group SX844).

A compound according to Compound (2G) wherein R¹ represents a methyl group, Q represents Q3, R³ represents a methoxy group, and a combination of structure of G² and substituents R^{X7}, R^{X8}, R^{X9} and R^{X10} applied to the structure of G² represents any combination described in Combination E (hereinafter, referred to as Compound group SX845).

Combination E consists of substituent numbers ZE1 to ZE196. Substituent numbers ZE1 to ZE196 represent combinations of structure of G² and substituents of R^{X7}, R^{X8}, R^{X9} and R^{X10} applied to the structure of G² according to Compound (2G). And hereinafter, they are referred to as [substituent number; G2, R^{X7}, R^{X8}, R^{X9}, R^{X10}]. For example, the substituent number ZE2 refers to a combination wherein G2 represents G1, R^{X7} represents a methyl group, and each of R^{X8}, R^{X9} and R^{X10} represents a hydrogen atom.
Combination E: [ZE1;G1,H,H,H,H], [ZE2;G1,Me,H,H,H], [ZE3;G1,Et,H,H,H], [ZE4;G1,Pr,H,H,H], [ZE5;G1,i-Pr,H,H,H], [ZE6;G1,c-Pr,H,H,H], [ZE7;G1,Ph,H,H,H], [ZE8;G1,OMe,H,H,H], [ZE9;G1,OEt,H,H,H], [ZE10;G1,OPh,H,H,H], [ZE11;G1,CF₃,H,H,H], [ZE12;G1,F,H,H,H], [ZE13;G1,Cl,H,H,H], [ZE14;G1,Br,H,H,H], [ZE15;G1,CN,H,H,H], [ZE16;G1,H,Me,H,H], [ZE17;G1,H,Et,H,H], [ZE18;G1,H,Pr,H,H], [ZE19;G1,H,i-Pr,H,H], [ZE20;G1,H,c-Pr,H,H], [ZE21;G1,H,Ph,H,H], [ZE22;G1,H,OMe,H,H], [ZE23;G1,H,OEt,H,H], [ZE24;G1,H,OPh,H,H], [ZE25;G1,H,CF₃,H,H], [ZE26;G1,H,F,H,H], [ZE27;G1,H,Cl,H,H], [ZE28;G1,H,Br,H,H], [ZE29;G1,H,CN,H,H], [ZE30;G1,H,H,Me,H], [ZE31;G1,H,H,Et,H], [ZE32;G1,H,H,Pr,H], [ZE33;G1,H,H,i-Pr,H], [ZE34;G1,H,H,c-Pr,H], [ZE35;G1,H,H,Ph,H], [ZE36;G1,H,H,OMe,H], [ZE37;G1,H,H,OEt,H], [ZE38;G1,H,H,OPh,H], [ZE39;G1,H,H,CF₃,H], [ZE40;G1,H,H,F,H], [ZE41;G1,H,H,Cl,H], [ZE42;G1,H,H,Br,H], [ZE43;G1,H,H,CN,H], [ZE44;G1,H,H,H,Me], [ZE45;G1,H,H,H,Et], [ZE46;G1,H,H,H,Pr], [ZE47;G1,H,H,H,i-Pr], [ZE48;G1,H,H,H,c-Pr], [ZE49;G1,H,H,H,Ph], [ZE50;G1,H,H,H,OMe], [ZE51;G1,H,H,H,OEt], [ZE52;G1,H,H,H,OPh], [ZE53;G1,H,H,H,CF₃], [ZE54;G1,H,H,H,F], [ZE55;G1,H,H,H,Cl], [ZE56;G1,H,H,H,Br], [ZE57;G1,H,H,H,CN], [ZE58;G9,H,H,H,-], [ZE59;G9,Me,H,H,-], [ZE60;G9,Et,H,H,-], [ZE61;G9,Pr,H,H,-], [ZE62;G9,i-Pr,H,H,-], [ZE63;G9,c-Pr,H,H,-], [ZE64;G9,Ph,H,H,-], [ZE65;G9,OMe,H,H,-], [ZE66;G9,OEt,H,H,-], [ZE67;G9,OPh,H,H,-], [ZE68;G9,CF₃,H,H, -], [ZE69;G9, F,H,H, -], [ZE70;G9,Cl,H,H, -], [ZE71;G9,Br,H,H,-], [ZE72;G9,CN,H,H,-], [ZE73;G9,H,Me,H,-], [ZE74;G9,H,Et,H,-], [ZE75;G9,H,Pr,H,-], [ZE76;G9,H,i-Pr,H,-], [ZE77;G9,H,c-Pr,H,-], [ZE78;G9,H,Ph,H,-], [ZE79;G9,H,OMe,H,-], [ZE80;G9,H,OEt,H,-], [ZE81;G9,H,OPh,H, -], [ZE82;G9,H,CF₃,H, -], [ZE83;G9,H, F,H, -], [ZE84;G9,H,Cl,H,-], [ZE85;G9,H,Br,H,-], [ZE86;G9,H,CN,H,-], [ZE87;G9,H,H,Me,-], [ZE88;G9,H,H,Et,-], [ZE89;G9,H,H,Pr,-], [ZE90;G9,H,H,i-Pr,-], [ZE91;G9,H,H,c-Pr,-], [ZE92;G9,H,H,Ph,-], [ZE93;G9,H,H,OMe,-], [ZE94;G9,H,H,OEt,-], [ZE95;G9,H,H,OPh,-], [ZE96;G9,H,H,CF₃,-], [ZE97;G9,H,H,F,-], [ZE98;G9,H,H,Cl,-], [ZE99;G9,H,H,Br,-], [ZE100;G9,H,H,CN,-], [ZE101;G10,H,H,H,-], [ZE102;G10,Me,H,H,-], [ZE103;G10,Et,H,H,-], [ZE104;G10,Pr,H,H,-], [ZE105;G10,i-Pr,H,H,-], [ZE106;G10,c-Pr,H,H,-], [ZE107;G10,Ph,H,H,-], [ZE108;G10,OMe,H,H,-], [ZE109;G10,OEt,H,H,-], [ZE110;G10,OPh,H,H, -], [ZE111;G10,CF₃,H,H, -], [ZE112;G10,F,H,H,-], [ZE113;G10,Cl,H,H,-], [ZE114;G10,Br,H,H,-], [ZE115;G10,CN,H,H,-], [ZE116;G10,H,Me,H,-], [ZE117;G10,H,Et,H,-], [ZE118;G10,H,Pr,H,-], [ZE119;G10,H,i-Pr,H,-], [ZE120;G10,H,c-Pr,H,-], [ZE121;G10,H,Ph,H,-], [ZE122;G10,H,OMe,H,-], [ZE123;G10,H,OEt,H,-], [ZE124;G10,H,OPh,H,-], [ZE125;G10,H,CF₃,H,-], [ZE126;G10,H,F,H,-], [ZE127;G10,H,Cl,H,-], [ZE128;G10,H,Br,H,-], [ZE129;G10,H,CN,H,-], [ZE130;G10,H,H,Me,-], [ZE131;G10,H,H,Et,-], [ZE132;G10,H,H,Pr,-], [ZE133;G10,H,H,i-Pr,-], [ZE134;G10,H,H,c-Pr,-], [ZE135;G10,H,H,Ph,-], [ZE136;G10,H,H,OMe,-], [ZE137;G10,H,H,OEt,-], [ZE138;G10,H,H,OPh, -], [ZE139;G10,H,H,CF₃, -], [ZE140;G10,H,H,F,-], [ZE141;G10,H,H,Cl,-], [ZE142;G10,H,H,Br,-], [ZE143;G10,H,H,CN,-], [ZE144;G30,H,H,H,-], [ZE145;G30,Me,H,H,-], [ZE146;G30,Et,H,H,-], [ZE147;G30,Pr,H,H,-], [ZE148;G30,i-Pr,H,H,-], [ZE149;G30,c-Pr,H,H,-], [ZE150;G30,Ph,H,H,-], [ZE151;G30,OMe,H,H,-], [ZE152;G30,OEt,H,H,-], [ZE153;G30,OPh,H,H, -], [ZE154;G30,CF₃,H,H, -], [ZE155;G30,F,H,H,-], [ZE156;G30,Cl,H,H,-], [ZE157;G30,Br,H,H,-], [ZE158;G30,CN,H,H,-], [ZE159;G30,H,Me,H,-], [ZE160;G30,H,Et,H,-], [ZE161;G30,H,Pr,H,-], [ZE162;G30,H,i-Pr,H,-], [ZE163;G30,H,c-Pr,H,-], [ZE164;G30,H,Ph,H,-], [ZE165;G30,H,OMe,H,-], [ZE166;G30,H,OEt,H,-], [ZE167;G30,H,OPh,H, -], [ZE168;G30,H,CF₃,H, -], [ZE169;G30,H,F,H,-], [ZE170;G30,H,Cl,H,-], [ZE171;G30,H,Br,H,-], [ZE172;G30,H,CN,H,-], [ZE173;G30,H,H,Me,-], [ZE174;G30,H,H,Et,-], [ZE175;G30,H,H,Pr,-], [ZE176;G30,H,H,i-Pr,-], [ZE177;G30,H,H,c-Pr,-], [ZE178;G30,H,H,Ph,-], [ZE179;G30,H,H,OMe,-], [ZE180;G30,H,H,OEt,-], [ZE181;G30,H,H,OPh,-], [ZE182;G30,H,H,CF₃,-], [ZE183;G30,H,H,F,-], [ZE184;G30,H,H,Cl,-], [ZE185;G30,H,H,Br,-], [ZE186;G30,H,H,CN,-], [ZE187;G30,H,Oi-Pr,H,-], [ZE188;G30,H,Oc-Pr,H,-], [ZE189;G30,H,Ot-Bu,H,-], [ZE190;G30,H,OCF₃,H,-], [ZE191;G30,H,OCHF₂,H, -], [ZE192;G30,H,OPr,H, -], [ZE193;G30,H,OCH2Ph,H,-], [ZE194;G30,H,OCH2-i-Pr,H,-], [ZE195;G30,H,OCH2-c-Pr,H,-], [ZE196;G30,H,OCH2-t-Bu,H,-]

A compound according to Compound (2G) wherein R¹ represents a chlorine atom, Q represents Q3, R³ represents a methoxy group, and a combination of structure of G² and substituents R^{X7}, R^{X8}, R^{X9} and R^{X10} applied to the structure of G² represents any combination described in Combination E (hereinafter, referred to as Compound group SX846).

A compound according to Compound (2G) wherein R¹ represents a methyl group, Q represents Q4, and a combination of structure of G² and substituents R^{X7}, R^{X8}, R^{X9} and R^{X10} applied to the structure of G² represents any combination described in Combination E (hereinafter, referred to as Compound group SX847).

A compound according to Compound (2G) wherein R¹ represents a chlorine atom, Q represents Q4, and a combination of structure of G² and substituents R^{X7}, R^{X8}, R^{X9} and R^{X10} applied to the structure of G² represents any combination described in Combination E (hereinafter, referred to as Compound group SX848).

A compound according to Compound (2G) wherein R¹ represents a methyl group, Q represents Q5, and a combination of structure of G² and substituents R^{X7}, R^{X8}, R^{X9} and R^{X10} applied to the structure of G² represents any combination described in Combination E (hereinafter, referred to as Compound group SX849).

A compound according to Compound (2G) wherein R¹ represents a chlorine atom, Q represents Q5, and a combination of structure of G² and substituents R^{X7}, R^{X8}, R^{X9} and R^{X10} applied to the structure of G² represents any combination described in Combination E (hereinafter, referred to as Compound group SX850).

A compound according to Compound (2G) wherein R¹ represents a methyl group, Q represents Q6, and a combination of structure of G² and substituents R^{X7}, R^{X8}, R^{X9} and R^{X10} applied to the structure of G² represents any combination described in Combination E (hereinafter, referred to as Compound group SX851).

A compound according to Compound (2G) wherein R¹ represents a chlorine atom, Q represents Q6, and a combination of structure of G² and substituents R^{X7}, R^{X8}, R^{X9} and R^{X10} applied to the structure of G² represents any combination described in Combination E (hereinafter, referred to as Compound group SX852).

A compound according to Compound (2G) wherein R¹ represents a methyl group, Q represents Q7, L² represents an oxygen atom, and a combination of structure of G² and substituents R^{X7}, R^{X8}, R^{X9} and R^{X10} applied to the structure of G² represents any combination described in Combination E (hereinafter, referred to as Compound group SX853).

A compound according to Compound (2G) wherein R¹ represents a chlorine atom, Q represents Q7, L² represents an oxygen atom, and a combination of structure of G² and substituents R^{X7}, R^{X8}, R^{X9} and R^{X10} applied to the structure of G² represents any combination described in Combination E (hereinafter, referred to as Compound group SX854).

A compound according to Compound (2G) wherein R¹ represents a methyl group, Q represents Q7, L² represents NH, and a combination of structure of G² and substituents R^{X7}, R^{X8}, R^{X9} and R^{X10} applied to the structure of G² represents any combination described in Combination E (hereinafter, referred to as Compound group SX855).

A compound according to Compound (2G) wherein R¹ represents a chlorine atom, Q represents Q7, L² represents NH, and a combination of structure of G² and substituents R^{X7}, R^{X8}, R^{X9} and R^{X10} applied to the structure of G² represents any combination described in Combination E (hereinafter, referred to as Compound group SX856).

A compound according to Compound (2G) wherein R¹ represents a methyl group, Q represents Q8A, and a combination of structure of G² and substituents R^{X7}, R^{X8}, R^{X9} and R^{X10} applied to the structure of G² represents any combination described in Combination E (hereinafter, referred to as Compound group SX857).

A compound according to Compound (2G) wherein R¹ represents a chlorine atom, Q represents Q8A, and a combination of structure of G² and substituents R^{X7}, R^{X8}, R^{X9} and R^{X10} applied to the structure of G² represents any combination described in Combination E (hereinafter, referred to as Compound group SX858).

A compound according to Compound (2G) wherein R¹ represents a methyl group, Q represents Q8D, and a combination of structure of G² and substituents R^{X7}, R^{X8}, R^{X9} and R^{X10} applied to the structure of G² represents any combination described in Combination E (hereinafter, referred to as Compound group SX859).

A compound according to Compound (2G) wherein R¹ represents a chlorine atom, Q represents Q8D, and a combination of structure of G² and substituents R^{X7}, R^{X8}, R^{X9} and R^{X10} applied to the structure of G² represents any combination described in Combination E (hereinafter, referred to as Compound group SX860).

A compound according to Compound (2G) wherein R¹ represents a methyl group, Q represents Q8J, and a combination of structure of G² and substituents R^{X7}, R^{X8}, R^{X9} and R^{X10} applied to the structure of G² represents any combination described in Combination E (hereinafter, referred to as Compound group SX861).

A compound according to Compound (2G) wherein R¹ represents a chlorine atom, Q represents Q8J, and a combination of structure of G² and substituents R^{X7}, R^{X8}, R^{X9} and R^{X10} applied to the structure of G² represents any combination described in Combination E (hereinafter, referred to as Compound group SX862).

A compound represented by formula (1K) (hereinafter, referred to as Compound (1K)):

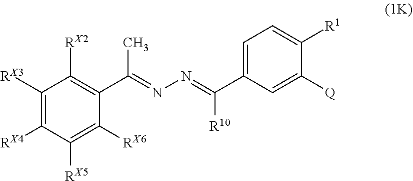

wherein R¹ represents a methyl group, Q represents Q1, R¹⁰ represents a hydrogen atom, and a combination of R^{X2}, R^{X3}, R^{X4}, R^{X5} and R^{X6} represents any combination described in Combination A (hereinafter, referred to as Compound group SX863).

A compound according to Compound (1K) wherein R¹ represents a chlorine atom, Q represents Q1, R¹⁰ represents a hydrogen atom, and a combination of R^{X2}, R^{X3}, R^{X4}, R^{X5} and R^{X6} represents any combination described in Combination A (hereinafter, referred to as Compound group SX864).

A compound according to Compound (1K) wherein R¹ represents an ethyl group, Q represents Q1, R¹⁰ represents a hydrogen atom, and a combination of R^{X2}, R^{X3}, R^{X4}, R^{X5} and R^{X6} represents any combination described in Combination A (hereinafter, referred to as Compound group SX865).

A compound according to Compound (1K) wherein R¹ represents a fluorine atom, Q represents Q1, R¹⁰ represents a hydrogen atom, and a combination of R^{X2}, R^{X3}, R^{X4}, R^{X5} and R^{X6} represents any combination described in Combination A (hereinafter, referred to as Compound group SX866).

A compound according to Compound (1K) wherein R¹ represents a cyclopropyl group, Q represents Q1, R¹⁰ represents a hydrogen atom, and a combination of R^{X2}, R^{X3}, R^{X4}, R^{X5} and R^{X6} represents any combination described in Combination A (hereinafter, referred to as Compound group SX867).

A compound according to Compound (1K) wherein R¹ represents a methyl group, Q represents Q2, L¹ represents an oxygen atom, R¹⁰ represents a hydrogen atom, and a combination of R^{X2}, R^{X3}, R^{X4}, R^{X5} and R^{X6} represents any combination described in Combination A (hereinafter, referred to as Compound group SX868).

A compound according to Compound (1K) wherein R¹ represents a chlorine atom, Q represents Q2, L¹ represents an oxygen atom, R¹⁰ represents a hydrogen atom, and a combination of R^{X2}, R^{X3}, R^{X4}, R^{X5} and R^{X6} represents any combination described in Combination A (hereinafter, referred to as Compound group SX869).

A compound according to Compound (1K) wherein R¹ represents a methyl group, Q represents Q2, L¹ represents NH, R¹⁰ represents a hydrogen atom, and a combination of R^{X2}, R^{X3}, R^{X4}, R^{X5} and R^{X6} represents any combination described in Combination A (hereinafter, referred to as Compound group SX870).

A compound according to Compound (1K) wherein R¹ represents a chlorine atom, Q represents Q2, L¹ represents NH, R¹⁰ represents a hydrogen atom, and a combination of R^{X2}, R^{X3}, R^{X4}, R^{X5} and R^{X6} represents any combination described in Combination A (hereinafter, referred to as Compound group SX871).

A compound according to Compound (1K) wherein R¹ represents a methyl group, Q represents Q3, R³ represents a methoxy group, R¹⁰ represents a hydrogen atom, and a combination of R^{X2}, R^{X3}, R^{X4}, R^{X5} and R^{X6} represents any combination described in Combination A (hereinafter, referred to as Compound group SX872).

A compound according to Compound (1K) wherein R¹ represents a chlorine atom, Q represents Q3, R³ represents a methoxy group, R¹⁰ represents a hydrogen atom, and a combination of R^{X2}, R^{X3}, R^{X4}, R^{X5} and R^{X6} represents any combination described in Combination A (hereinafter, referred to as Compound group SX873).

A compound according to Compound (1K) wherein R¹ represents a methyl group, Q represents Q4, R¹⁰ represents a hydrogen atom, and a combination of R^{X2}, R^{X3}, R^{X4}, R^{X5} and R^{X6} represents any combination described in Combination A (hereinafter, referred to as Compound group SX874).

A compound according to Compound (1K) wherein R¹ represents a chlorine atom, Q represents Q4, R¹⁰ represents a hydrogen atom, and a combination of R^{X2}, R^{X3}, R^{X4}, R^{X5} and R^{X6} represents any combination described in Combination A (hereinafter, referred to as Compound group SX875).

A compound according to Compound (1K) wherein R¹ represents a methyl group, Q represents Q5, R¹⁰ represents a hydrogen atom, and a combination of R^{X2}, R^{X3}, R^{X4}, R^{X5} and R^{X6} represents any combination described in Combination A (hereinafter, referred to as Compound group SX876).

A compound according to Compound (1K) wherein R¹ represents a chlorine atom, Q represents Q5, R¹⁰ represents a hydrogen atom, and a combination of R^{X2}, R^{X3}, R^{X4}, R^{X5} and R^{X6} represents any combination described in Combination A (hereinafter, referred to as Compound group SX877).

A compound according to Compound (1K) wherein R¹ represents a methyl group, Q represents Q6, R¹⁰ represents a hydrogen atom, and a combination of R^{X2}, R^{X3}, R^{X4}, R^{X5} and R^{X6} represents any combination described in Combination A (hereinafter, referred to as Compound group SX878).

A compound according to Compound (1K) wherein R¹ represents a chlorine atom, Q represents Q6, R¹⁰ represents a hydrogen atom, and a combination of R^{X2}, R^{X3}, R^{X4}, R^{X5} and R^{X6} represents any combination described in Combination A (hereinafter, referred to as Compound group SX879).

A compound according to Compound (1K) wherein R¹ represents a methyl group, Q represents Q7, L² represents an oxygen atom, R¹⁰ represents a hydrogen atom, and a combination of R^{X2}, R^{X3}, R^{X4}, R^{X5} and R^{X6} represents any combination described in Combination A (hereinafter, referred to as Compound group SX880).

A compound according to Compound (1K) wherein R¹ represents a chlorine atom, Q represents Q7, L² represents an oxygen atom, R¹⁰ represents a hydrogen atom, and a combination of R^{X2}, R^{X3}, R^{X4}, R^{X5} and R^{X6} represents any combination described in Combination A (hereinafter, referred to as Compound group SX881).

A compound according to Compound (1K) wherein R¹ represents a methyl group, Q represents Q7, L² represents NH, R¹⁰ represents a hydrogen atom, and a combination of R^{X2}, R^{X3}, R^{X4}, R^{X5} and R^{X6} represents any combination described in Combination A (hereinafter, referred to as Compound group SX882).

A compound according to Compound (1K) wherein R¹ represents a chlorine atom, Q represents Q7, L² represents NH, R¹⁰ represents a hydrogen atom, and a combination of R^{X2}, R^{X3}, R^{X4}, R^{X5} and R^{X6} represents any combination described in Combination A (hereinafter, referred to as Compound group SX883).

A compound according to Compound (1K) wherein R¹ represents a methyl group, Q represents Q8A, R¹⁰ represents a hydrogen atom, and a combination of R^{X2}, R^{X3}, R^{X4}, R^{X5} and R^{X6} represents any combination described in Combination A (hereinafter, referred to as Compound group SX884).

A compound according to Compound (1K) wherein R¹ represents a chlorine atom, Q represents Q8A, R¹⁰ represents a hydrogen atom, and a combination of R^{X2}, R^{X3}, R^{X4}, R^{X5} and R^{X6} represents any combination described in Combination A (hereinafter, referred to as Compound group SX885).

A compound according to Compound (1K) wherein R¹ represents a methyl group, Q represents Q8D, R¹⁰ represents a hydrogen atom, and a combination of R^{X2}, R^{X3}, R^{X4}, R^{X5} and R^{X6} represents any combination described in Combination A (hereinafter, referred to as Compound group SX886).

A compound according to Compound (1K) wherein R¹ represents a chlorine atom, Q represents Q8D, R¹⁰ represents a hydrogen atom, and a combination of R^{X2}, R^{X3}, R^{X4}, R^{X5} and R^{X6} represents any combination described in Combination A (hereinafter, referred to as Compound group SX887).

A compound according to Compound (1K) wherein R¹ represents a methyl group, Q represents Q8J, R¹⁰ represents a hydrogen atom, and a combination of R^{X2}, R^{X3}, R^{X4}, R^{X5} and R^{X6} represents any combination described in Combination A (hereinafter, referred to as Compound group SX888).

A compound according to Compound (1K) wherein R¹ represents a chlorine atom, Q represents Q8J, R¹⁰ represents a hydrogen atom, and a combination of R^{X2}, R^{X3}, R^{X4}, R^{X5} and R^{X6} represents any combination described in Combination A (hereinafter, referred to as Compound group SX889).

A compound according to Compound (1K) wherein R¹ represents a methyl group, Q represents Q3, R³ represents a methoxy group, R¹⁰ represents a methyl group, and a combination of R^{X2}, R^{X3}, R^{X4}, R^{X5} and R^{X6} represents any combination described in Combination A (hereinafter, referred to as Compound group SX890).

A compound according to Compound (1K) wherein R¹ represents a chlorine atom, Q represents Q3, R³ represents a methoxy group, R¹⁰ represents a methyl group, and a combination of R^{X2}, R^{X3}, R^{X4}, R^{X5} and R^{X6} represents any combination described in Combination A (hereinafter, referred to as Compound group SX891).

A compound according to Compound (1K) wherein R¹ represents a methyl group, Q represents Q4, R¹⁰ represents a methyl group, and a combination of R^{X2}, R^{X3}, R^{X4}, R^{X5} and R^{X6} represents any combination described in Combination A (hereinafter, referred to as Compound group SX892).

A compound according to Compound (1K) wherein R¹ represents a chlorine atom, Q represents Q4, R¹⁰ represents a methyl group, and a combination of R^{X2}, R^{X3}, R^{X4}, R^{X5} and R^{X6} represents any combination described in Combination A (hereinafter, referred to as Compound group SX893).

A compound according to Compound (1K) wherein R¹ represents a methyl group, Q represents Q5, R¹⁰ represents a methyl group, and a combination of R^{X2}, R^{X3}, R^{X4}, R^{X5} and R^{X6} represents any combination described in Combination A (hereinafter, referred to as Compound group SX894).

A compound according to Compound (1K) wherein R¹ represents a chlorine atom, Q represents Q5, R¹⁰ represents a methyl group, and a combination of R^{X2}, R^{X3}, R^{X4}, R^{X5} and R^{X6} represents any combination described in Combination A (hereinafter, referred to as Compound group SX895).

A compound according to Compound (1K) wherein R¹ represents a methyl group, Q represents Q6, R¹⁰ represents a methyl group, and a combination of R^{X2}, R^{X3}, R^{X4}, R^{X5} and R^{X6} represents any combination described in Combination A (hereinafter, referred to as Compound group SX896).

A compound according to Compound (1K) wherein R¹ represents a chlorine atom, Q represents Q6, R¹⁰ represents a methyl group, and a combination of R^{X2}, R^{X3}, R^{X4}, R^{X5} and R^{X6} represents any combination described in Combination A (hereinafter, referred to as Compound group SX897).

A compound according to Compound (1K) wherein R¹ represents a methyl group, Q represents Q7, L² represents an oxygen atom, R¹⁰ represents a methyl group, and a combination of R^{X2}, R^{X3}, R^{X4}, R^{X5} and R^{X6} represents any combination described in Combination A (hereinafter, referred to as Compound group SX898).

A compound according to Compound (1K) wherein R¹ represents a chlorine atom, Q represents Q7, L² represents an oxygen atom, R¹⁰ represents a methyl group, and a combination of R^{X2}, R^{X3}, R^{X4}, R^{X5} and R^{X6} represents any combination described in Combination A (hereinafter, referred to as Compound group SX899).

A compound according to Compound (1K) wherein R¹ represents a methyl group, Q represents Q7, L² represents NH, R¹⁰ represents a methyl group, and a combination of R^{X2}, R^{X3}, R^{X4}, R^{X5} and R^{X6} represents any combination described in Combination A (hereinafter, referred to as Compound group SX900).

A compound according to Compound (1K) wherein R¹ represents a chlorine atom, Q represents Q7, L² represents NH, R¹⁰ represents a methyl group, and a combination of R^{X2}, R^{X3}, R^{X4}, R^{X5} and R^{X6} represents any combination described in Combination A (hereinafter, referred to as Compound group SX901).

A compound according to Compound (1K) wherein R¹ represents a methyl group, Q represents Q8A, R¹⁰ represents a methyl group, and a combination of R^{X2}, R^{X3}, R^{X4}, R^{X5} and R^{X6} represents any combination described in Combination A (hereinafter, referred to as Compound group SX902).

A compound according to Compound (1K) wherein R¹ represents a chlorine atom, Q represents Q8A, R¹⁰ represents a methyl group, and a combination of R^{X2}, R^{X3}, R^{X4}, R^{X5} and R^{X6} represents any combination described in Combination A (hereinafter, referred to as Compound group SX903).

A compound according to Compound (1K) wherein R¹ represents a methyl group, Q represents Q8D, R¹⁰ represents a methyl group, and a combination of R^{X2}, R^{X3}, R^{X4}, R^{X5} and R^{X6} represents any combination described in Combination A (hereinafter, referred to as Compound group SX904).

A compound according to Compound (1K) wherein R¹ represents a chlorine atom, Q represents Q8D, R¹⁰ represents a methyl group, and a combination of R^{X2}, R^{X3}, R^{X4}, R^{X5} and R^{X6} represents any combination described in Combination A (hereinafter, referred to as Compound group SX905).

A compound according to Compound (1K) wherein R¹ represents a methyl group, Q represents Q8J, R¹⁰ represents a methyl group, and a combination of R^{X2}, R^{X3}, R^{X4}, R^{X5} and R^{X6} represents any combination described in Combination A (hereinafter, referred to as Compound group SX906).

A compound according to Compound (1K) wherein R¹ represents a chlorine atom, Q represents Q8J, R¹⁰ represents a methyl group, and a combination of R^{X2}, R^{X3}, R^{X4}, R^{X5} and R^{X6} represents any combination described in Combination A (hereinafter, referred to as Compound group SX907).

A compound according to Compound (1K) wherein R¹ represents a methyl group, Q represents Q3, R³ represents a methoxy group, R¹⁰ represents an ethyl group, and a combination of R^{X2}, R^{X3}, R^{X4}, R^{X5} and R^{X6} represents any combination described in Combination A (hereinafter, referred to as Compound group SX908).

A compound according to Compound (1K) wherein R¹ represents a chlorine atom, Q represents Q3, R³ represents a methoxy group, R¹⁰ represents an ethyl group, and a combination of R^{X2}, R^{X3}, R^{X4}, R^{X5} and R^{X6} represents any combination described in Combination A (hereinafter, referred to as Compound group SX909).

A compound according to Compound (1K) wherein R¹ represents a methyl group, Q represents Q4, R¹⁰ represents an ethyl group, and a combination of R^{X2}, R^{X3}, R^{X4}, R^{X5} and R^{X6} represents any combination described in Combination A (hereinafter, referred to as Compound group SX910).

A compound according to Compound (1K) wherein R¹ represents a chlorine atom, Q represents Q4, R¹⁰ represents an ethyl group, and a combination of R^{X2}, R^{X3}, R^{X4}, R^{X5} and R^{X6} represents any combination described in Combination A (hereinafter, referred to as Compound group SX911).

A compound according to Compound (1K) wherein R¹ represents a methyl group, Q represents Q5, R¹⁰ represents an ethyl group, and a combination of R^{X2}, R^{X3}, R^{X4}, R^{X5} and R^{X6} represents any combination described in Combination A (hereinafter, referred to as Compound group SX912).

A compound according to Compound (1K) wherein R¹ represents a chlorine atom, Q represents Q5, R¹⁰ represents an ethyl group, and a combination of R^{X2}, R^{X3}, R^{X4}, R^{X5} and R^{X6} represents any combination described in Combination A (hereinafter, referred to as Compound group SX913).

A compound according to Compound (1K) wherein R¹ represents a methyl group, Q represents Q6, R¹⁰ represents an ethyl group, and a combination of R^{X2}, R^{X3}, R^{X4}, R^{X5} and R^{X6} represents any combination described in Combination A (hereinafter, referred to as Compound group SX914).

A compound according to Compound (1K) wherein R¹ represents a chlorine atom, Q represents Q6, R¹⁰ represents an ethyl group, and a combination of R^{X2}, R^{X3}, R^{X4}, R^{X5} and R^{X6} represents any combination described in Combination A (hereinafter, referred to as Compound group SX915).

A compound according to Compound (1K) wherein R¹ represents a methyl group, Q represents Q7, L² represents an oxygen atom, R¹⁰ represents an ethyl group, and a combination of R^{X2}, R^{X3}, R^{X4}, R^{X5} and R^{X6} represents any combination described in Combination A (hereinafter, referred to as Compound group SX916).

A compound according to Compound (1K) wherein R¹ represents a chlorine atom, Q represents Q7, L² represents an oxygen atom, R¹⁰ represents an ethyl group, and a combination of R^{X2}, R^{X3}, R^{X4}, R^{X5} and R^{X6} represents any combination described in Combination A (hereinafter, referred to as Compound group SX917).

A compound according to Compound (1K) wherein R¹ represents a methyl group, Q represents Q7, L² represents NH, R¹⁰ represents an ethyl group, and a combination of R^{X2}, R^{X3}, R^{X4}, R^{X5} and R^{X6} represents any combination described in Combination A (hereinafter, referred to as Compound group SX918).

A compound according to Compound (1K) wherein R¹ represents a chlorine atom, Q represents Q7, L² represents NH, R¹⁰ represents an ethyl group, and a combination of R^{X2}, R^{X3}, R^{X4}, R^{X5} and R^{X6} represents any combination described in Combination A (hereinafter, referred to as Compound group SX919).

A compound according to Compound (1K) wherein R¹ represents a methyl group, Q represents Q8A, R¹⁰ represents an ethyl group, and a combination of R^{X2}, R^{X3}, R^{X4}, R^{X5} and R^{X6} represents any combination described in Combination A (hereinafter, referred to as Compound group SX920).

A compound according to Compound (1K) wherein R¹ represents a chlorine atom, Q represents Q8A, R¹⁰ represents an ethyl group, and a combination of R^{X2}, R^{X3}, R^{X4}, R^{X5} and R^{X6} represents any combination described in Combination A (hereinafter, referred to as Compound group SX921).

A compound according to Compound (1K) wherein R¹ represents a methyl group, Q represents Q8D, R¹⁰ represents an ethyl group, and a combination of R^{X2}, R^{X3}, R^{X4}, R^{X5} and R^{X6} represents any combination described in Combination A (hereinafter, referred to as Compound group SX922).

A compound according to Compound (1K) wherein R¹ represents a chlorine atom, Q represents Q8D, R¹⁰ represents an ethyl group, and a combination of R^{X2}, R^{X3}, R^{X4}, R^{X5} and R^{X6} represents any combination described in Combination A (hereinafter, referred to as Compound group SX923).

A compound according to Compound (1K) wherein R¹ represents a methyl group, Q represents Q8J, R¹⁰ represents an ethyl group, and a combination of R^{X2}, R^{X3}, R^{X4}, R^{X5} and R^{X6} represents any combination described in Combination A (hereinafter, referred to as Compound group SX924).

A compound according to Compound (1K) wherein R¹ represents a chlorine atom, Q represents Q8J, R¹⁰ represents an ethyl group, and a combination of R^{X2}, R^{X3}, R^{X4}, R^{X5} and R^{X6} represents any combination described in Combination A (hereinafter, referred to as Compound group SX925).

A compound according to Compound (1K) wherein R¹ represents a methyl group, Q represents Q3, R³ represents a methoxy group, R¹⁰ represents a cyclopropyl group, and a combination of R^{X2}, R^{X3}, R^{X4}, R^{X5} and R^{X6} represents any combination described in Combination A (hereinafter, referred to as Compound group SX926).

A compound according to Compound (1K) wherein R¹ represents a chlorine atom, Q represents Q3, R³ represents a methoxy group, R¹⁰ represents a cyclopropyl group, and a combination of R^{X2}, R^{X3}, R^{X4}, R^{X5} and R^{X6} represents any combination described in Combination A (hereinafter, referred to as Compound group SX927).

A compound according to Compound (1K) wherein R¹ represents a methyl group, Q represents Q4, R¹⁰ represents a cyclopropyl group, and a combination of R^{X2}, R^{X3}, R^{X4}, R^{X5} and R^{X6} represents any combination described in Combination A (hereinafter, referred to as Compound group SX928).

A compound according to Compound (1K) wherein R¹ represents a chlorine atom, Q represents Q4, R¹⁰ represents a cyclopropyl group, and a combination of R^{X2}, R^{X3}, R^{X4}, R^{X5} and R^{X6} represents any combination described in Combination A (hereinafter, referred to as Compound group SX929).

A compound according to Compound (1K) wherein R¹ represents a methyl group, Q represents Q5, R¹⁰ represents a cyclopropyl group, and a combination of R^{X2}, R^{X3}, R^{X4}, R^{X5} and R^{X6} represents any combination described in Combination A (hereinafter, referred to as Compound group SX930).

A compound according to Compound (1K) wherein R¹ represents a chlorine atom, Q represents Q5, R¹⁰ represents a cyclopropyl group, and a combination of R^{X2}, R^{X3}, R^{X4}, R^{X5} and R^{X6} represents any combination described in Combination A (hereinafter, referred to as Compound group SX931).

A compound according to Compound (1K) wherein R¹ represents a methyl group, Q represents Q6, R¹⁰ represents a cyclopropyl group, and a combination of R^{X2}, R^{X3}, R^{X4}, R^{X5} and R^{X6} represents any combination described in Combination A (hereinafter, referred to as Compound group SX932).

A compound according to Compound (1K) wherein R¹ represents a chlorine atom, Q represents Q6, R¹⁰ represents a cyclopropyl group, and a combination of R^{X2}, R^{X3}, R^{X4}, R^{X5} and R^{X6} represents any combination described in Combination A (hereinafter, referred to as Compound group SX933).

A compound according to Compound (1K) wherein R¹ represents a methyl group, Q represents Q7, L² represents an oxygen atom, R¹⁰ represents a cyclopropyl group, and a combination of R^{X2}, R^{X3}, R^{X4}, R^{X5} and R^{X6} represents any combination described in Combination A (hereinafter, referred to as Compound group SX34).

A compound according to Compound (1K) wherein R¹ represents a chlorine atom, Q represents Q7, L² represents an oxygen atom, R¹⁰ represents a cyclopropyl group, and a combination of R^{X2}, R^{X3}, R^{X4}, R^{X5} and R^{X6} represents any combination described in Combination A (hereinafter, referred to as Compound group SX935).

A compound according to Compound (1K) wherein R¹ represents a methyl group, Q represents Q7, L² represents NH, R¹⁰ represents a cyclopropyl group, and a combination of R^{X2}, R^{X3}, R^{X4}, R^{X5} and R^{X6} represents any combination described in Combination A (hereinafter, referred to as Compound group SX936).

A compound according to Compound (1K) wherein R¹ represents a chlorine atom, Q represents Q7, L² represents NH, R¹⁰ represents a cyclopropyl group, and a combination of R^{X2}, R^{X3}, R^{X4}, R^{X5} and R^{X6} represents any combination described in Combination A (hereinafter, referred to as Compound group SX937).

A compound according to Compound (1K) wherein R¹ represents a methyl group, Q represents Q8A, R¹⁰ represents a cyclopropyl group, and a combination of R^{X2}, R^{X3}, R^{X4}, R^{X5} and R^{X6} represents any combination described in Combination A (hereinafter, referred to as Compound group SX938).

A compound according to Compound (1K) wherein R¹ represents a chlorine atom, Q represents Q8A, R¹⁰ represents a cyclopropyl group, and a combination of R^{X2}, R^{X3}, R^{X4}, R^{X5} and R^{X6} represents any combination described in Combination A (hereinafter, referred to as Compound group SX939).

A compound according to Compound (1K) wherein R¹ represents a methyl group, Q represents Q8D, R¹⁰ represents a cyclopropyl group, and a combination of R^{X2}, R^{X3}, R^{X4}, R^{X5} and R^{X6} represents any combination described in Combination A (hereinafter, referred to as Compound group SX940).

A compound according to Compound (1K) wherein R¹ represents a chlorine atom, Q represents Q8D, R¹⁰ represents a cyclopropyl group, and a combination of R^{X2}, R^{X3}, R^{X4}, R^{X5} and R^{X6} represents any combination described in Combination A (hereinafter, referred to as Compound group SX941).

A compound according to Compound (1K) wherein R¹ represents a methyl group, Q represents Q8J, R¹⁰ represents a cyclopropyl group, and a combination of R^{X2}, R^{X3}, R^{X4}, R^{X5} and R^{X6} represents any combination described in Combination A (hereinafter, referred to as Compound group SX942).

A compound according to Compound (1K) wherein R¹ represents a chlorine atom, Q represents Q8J, R¹⁰ represents a cyclopropyl group, and a combination of R^{X2}, R^{X3}, R^{X4}, R^{X5} and R^{X6} represents any combination described in Combination A (hereinafter, referred to as Compound group SX943).

A compound according to Compound (1K) wherein R¹ represents a methyl group, Q represents Q1, R¹⁰ represents a methyl group, and a combination of R^{X2}, R^{X3}, R^{X4}, R^{X5} and R^{X6} represents any combination described in Combination A (hereinafter, referred to as Compound group SX944).

A compound according to Compound (1K) wherein R¹ represents a chlorine atom, Q represents Q1, R¹⁰ represents a methyl group, and a combination of R^{X2}, R^{X3}, R^{X4}, R^{X5} and R^{X6} represents any combination described in Combination A (hereinafter, referred to as Compound group SX945).

A compound according to Compound (1K) wherein R¹ represents an ethyl group, Q represents Q1, R¹⁰ represents a methyl group, and a combination of R^{X2}, R^{X3}, R^{X4}, R^{X5} and R^{X6} represents any combination described in Combination A (hereinafter, referred to as Compound group SX946).

A compound according to Compound (1K) wherein R¹ represents a fluorine atom, Q represents Q1, R¹⁰ represents a methyl group, and a combination of R^{X2}, R^{X3}, R^{X4}, R^{X5} and R^{X6} represents any combination described in Combination A (hereinafter, referred to as Compound group SX947).

A compound according to Compound (1K) wherein R¹ represents a cyclopropyl group, Q represents Q1, R¹⁰ represents a methyl group, and a combination of R^{X2}, R^{X3}, R^{X4}, R^{X5} and R^{X6} represents any combination described in Combination A (hereinafter, referred to as Compound group SX948).

A compound according to Compound (1K) wherein R¹ represents a methyl group, Q represents Q2, L¹ represents an oxygen atom, R¹⁰ represents a methyl group, and a combination of R^{X2}, R^{X3}, R^{X4}, R^{X5} and R^{X6} represents any combination described in Combination A (hereinafter, referred to as Compound group SX949).

A compound according to Compound (1K) wherein R¹ represents a chlorine atom, Q represents Q2, L¹ represents an oxygen atom, R¹⁰ represents a methyl group, and a combination of R^{X2}, R^{X3}, R^{X4}, R^{X5} and R^{X6} represents any combination described in Combination A (hereinafter, referred to as Compound group SX950).

A compound according to Compound (1K) wherein R¹ represents a methyl group, Q represents Q2, L¹ represents NH, R¹⁰ represents a methyl group, and a combination of R^{X2}, R^{X3}, R^{X4}, R^{X5} and R^{X6} represents any combination described in Combination A (hereinafter, referred to as Compound group SX951).

A compound according to Compound (1K) wherein R¹ represents a chlorine atom, Q represents Q2, L¹ represents NH, R¹⁰ represents a methyl group, and a combination of R^{X2}, R^{X3}, R^{X4}, R^{X5} and R^{X6} represents any combination described in Combination A (hereinafter, referred to as Compound group SX952).

A compound according to Compound (1K) wherein R¹ represents a methyl group, Q represents Q1, R¹⁰ represents an ethyl group, and a combination of R^{X2}, R^{X3}, R^{X4}, R^{X5} and R^{X6} represents any combination described in Combination A (hereinafter, referred to as Compound group SX953).

A compound according to Compound (1K) wherein R¹ represents a chlorine atom, Q represents Q1, R¹⁰ represents an ethyl group, and a combination of R^{X2}, R^{X3}, R^{X4}, R^{X5} and R^{X6} represents any combination described in Combination A (hereinafter, referred to as Compound group SX954).

A compound according to Compound (1K) wherein R¹ represents an ethyl group, Q represents Q1, R¹⁰ represents an ethyl group, and a combination of R^{X2}, R^{X3}, R^{X4}, R^{X5} and R^{X6} represents any combination described in Combination A (hereinafter, referred to as Compound group SX955).

A compound according to Compound (1K) wherein R¹ represents a fluorine atom, Q represents Q1, R¹⁰ represents an ethyl group, and a combination of R^{X2}, R^{X3}, R^{X4}, R^{X5} and R^{X6} represents any combination described in Combination A (hereinafter, referred to as Compound group SX956).

A compound according to Compound (1K) wherein R¹ represents a cyclopropyl group, Q represents Q1, R¹⁰ represents an ethyl group, and a combination of R^{X2}, R^{X3}, R^{X4}, R^{X5} and R^{X6} represents any combination described in Combination A (hereinafter, referred to as Compound group SX957).

A compound according to Compound (1K) wherein R¹ represents a methyl group, Q represents Q2, L¹ represents an oxygen atom, R¹⁰ represents an ethyl group, and a combination of R^{X2}, R^{X3}, R^{X4}, R^{X5} and R^{X6} represents any combination described in Combination A (hereinafter, referred to as Compound group SX958).

A compound according to Compound (1K) wherein R¹ represents a chlorine atom, Q represents Q2, L¹ represents an oxygen atom, R¹⁰ represents an ethyl group, and a combination of R^{X2}, R^{X3}, R^{X4}, R^{X5} and R^{X6} represents any combination described in Combination A (hereinafter, referred to as Compound group SX959).

A compound according to Compound (1K) wherein R¹ represents a methyl group, Q represents Q2, L¹ represents NH, R¹⁰ represents an ethyl group, and a combination of R^{X2}, R^{X3}, R^{X4}, R^{X5} and R^{X6} represents any combination described in Combination A (hereinafter, referred to as Compound group SX960).

A compound according to Compound (1K) wherein R¹ represents a chlorine atom, Q represents Q2, L¹ represents NH, R¹⁰ represents an ethyl group, and a combination of R^{X2}, R^{X3}, R^{X4}, R^{X5} and R^{X6} represents any combination described in Combination A (hereinafter, referred to as Compound group SX961).

A compound according to Compound (1K) wherein R¹ represents a methyl group, Q represents Q1, R¹⁰ represents a cyclopropyl group, and a combination of R^{X2}, R^{X3}, R^{X4}, R^{X5} and R^{X6} represents any combination described in Combination A (hereinafter, referred to as Compound group SX962).

A compound according to Compound (1K) wherein R¹ represents a chlorine atom, Q represents Q1, R¹⁰ represents a cyclopropyl group, and a combination of R^{X2}, R^{X3}, R^{X4}, R^{X5} and R^{X6} represents any combination described in Combination A (hereinafter, referred to as Compound group SX963).

A compound according to Compound (1K) wherein R¹ represents an ethyl group, Q represents Q1, R¹⁰ represents a cyclopropyl group, and a combination of R^{X2}, R^{X3}, R^{X4}, R^{X5} and R^{X6} represents any combination described in Combination A (hereinafter, referred to as Compound group SX964).

A compound according to Compound (1K) wherein R¹ represents a fluorine atom, Q represents Q1, R¹⁰ represents a cyclopropyl group, and a combination of R^{X2}, R^{X3}, R^{X4}, R^{X5} and R^{X6} represents any combination described in Combination A (hereinafter, referred to as Compound group SX965).

A compound according to Compound (1K) wherein R¹ represents a cyclopropyl group, Q represents Q1, R¹⁰ represents a cyclopropyl group, and a combination of R^{X2}, R^{X3}, R^{X4}, R^{X5} and R^{X6} represents any combination described in Combination A (hereinafter, referred to as Compound group SX966).

A compound according to Compound (1K) wherein R¹ represents a methyl group, Q represents Q2, L¹ represents an oxygen atom, R¹⁰ represents a cyclopropyl group, and a combination of R^{X2}, R^{X3}, R^{X4}, R^{X5} and R^{X6} represents any combination described in Combination A (hereinafter, referred to as Compound group SX967).

A compound according to Compound (1K) wherein R¹ represents a chlorine atom, Q represents Q2, L¹ represents an oxygen atom, R¹⁰ represents a cyclopropyl group, and a combination of R^{X2}, R^{X3}, R^{X4}, R^{X5} and R^{X6} represents any combination described in Combination A (hereinafter, referred to as Compound group SX968).

A compound according to Compound (1K) wherein R¹ represents a methyl group, Q represents Q2, L¹ represents NH, R¹⁰ represents a cyclopropyl group, and a combination of R^{X2}, R^{X3}, R^{X4}, R^{X5} and R^{X6} represents any combination described in Combination A (hereinafter, referred to as Compound group SX969).

A compound according to Compound (1K) wherein R¹ represents a chlorine atom, Q represents Q2, L¹ represents NH, R¹⁰ represents a cyclopropyl group, and a combination of R^{X2}, R^{X3}, R^{X4}, R^{X5} and R^{X6} represents any combination described in Combination A (hereinafter, referred to as Compound group SX970).

A compound represented by formula (1L) (hereinafter, referred to as Compound (1L)):

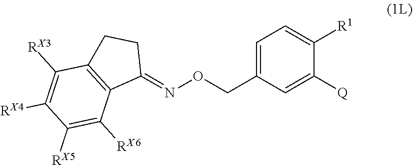

wherein R¹ represents a methyl group, Q represents Q1, and a combination of R^{X3}, R^{X4}, R^{X5} and R^{X6} represents any combination described in Combination F (hereinafter, referred to as Compound group SX971).

Combination F consists of substituent numbers ZF1 to ZF145. Substituent numbers ZF1 to ZF145 represent combinations of R^{X3}, R^{X4}, R^{X5} and R^{X6} according to Compound (1L), compounds represented by formula (10), compounds represented by formula (1Q), compounds represented by formula (1R), compounds represented by formula (1S) and compounds represented by formula (1T). And hereinafter, they are referred to as [substituent number; R^{X3},R^{X4},R^{X5},R^{X6}]. For example, the substituent number ZF2 refers to a combination wherein R^{X3} represents a methyl group, and each of R^{X4}, R^{X5} and R^{X6} represents a hydrogen atom.
Combination F: [ZF1;H,H,H,H], [ZF2;Me,H,H,H], [ZF3;Et,H,H,H], [ZF4;Pr,H,H,H], [ZF5;i-Pr,H,H,H], [ZF6;c-Pr,H,H,H], [ZF7;OMe,H,H,H], [ZF8;OEt,H,H,H], [ZF9;CF₃,H,H,H], [ZF10;F,H,H,H], [ZF11;Cl,H,H,H], [ZF12;Br,H,H,H], [ZF13;CN,H,H, H], [ZF14;H,Me,H,H], [ZF15;H,Et,H,H], [ZF16;H,Pr,H,H], [ZF17;H, i-Pr,H,H], [ZF18;H,c-Pr,H,H], [ZF19;H,OMe,H,H], [ZF20;H,OEt,H,H], [ZF21;H,CF₃,H,H], [ZF22;H,F,H,H], [ZF23;H,Cl,H,H], [ZF24;H,Br,H,H], [ZF25;H,CN,H,H], [ZF26;H,H,Me,H], [ZF27;H,H,Et,H], [ZF28;H,H,Pr,H], [ZF29;H,H,i-Pr,H], [ZF30;H,H,c-Pr,H], [ZF31;H,H,OMe,H], [ZF32;H,H,OEt,H], [ZF33;H,H,CF₃,H], [Z F34;H,H,F,H], [ZF35;H,H,Cl,H], [ZF36;H,H,Br,H], [ZF37;H,H,CN,H], [ZF38;H,H,H,Me], [ZF39;H,H,H,Et], [ZF40;H,H,H,Pr], [ZF41;H,H,H,i-Pr], [ZF42;H,H,H,c-Pr], [ZF43;H,H,H,OMe], [ZF44;H,H,H,OEt], [ZF45;H,H,H,CF₃], [ZF4 6;H,H,H,F], [ZF47;H,H,H,Cl], [ZF48;H,H,H,Br], [ZF49;H,H,H,CN], [ZF50;Me,Me,H,H], [ZF51;Me,OMe,H,H], [ZF52;Me,F,H,H], [ZF53;Me,Cl,H,H], [ZF54;Me,H,Me,H], [ZF55;Me,H,OMe,H], [ZF56;Me,H,F,H], [ZF57;Me,H,Cl,H], [ZF58;Me,H,H,Me], [ZF59;Me,H,H,OMe], [ZF60; Me,H,H,F], [ZF61;Me,H,H,Cl], [ZF62;OMe,Me,H,H], [ZF63;OMe,OMe, H,H], [ZF64;OMe,F,H,H], [ZF65;OMe,Cl,H,H], [ZF66;OMe,H,Me,H], [ZF67;OMe,H,OMe,H], [ZF68;OMe,H,F,H], [ZF69;OMe,H,Cl,H], [ZF70; OMe,H,H,Me], [ZF71;OMe,H,H,OMe], [ZF72;OMe,H,H,F], [ZF73;OMe,H,H,Cl], [ZF74;F,Me,H,H], [ZF75;F,OMe,H,H], [ZF76;F,F,H,H], [ZF7 7;F,Cl,H,H], [ZF78;F,H,Me,H], [ZF79;F,H,OMe,H], [ZF80;F,H,F,H], [ZF81;F,H,Cl,H], [ZF82;F,H,H,Me], [ZF83;F,H,H,OMe], [ZF84;F,H,H,F], [ZF85;F,H,H,Cl], [ZF86;Cl,Me,H,H], [ZF87;Cl,OMe,H,H], [Z F88;Cl,F,H,H], [ZF89;Cl,Cl,H,H], [ZF90;Cl,H,Me,H], [ZF91;Cl,H, OMe,H], [ZF92;Cl,H,F,H], [ZF93;Cl,H,Cl,H], [ZF94;Cl,H,H,Me], [Z F95;Cl,H,H,OMe], [ZF96;Cl,H,H,F], [ZF97;Cl,H,H,Cl], [ZF98;H,Me,Me,H], [ZF99;H,Me,OMe,H], [ZF100;H,Me,F,H], [ZF101;H,Me,Cl,H], [ZF102;H,Me,H,Me], [ZF103;H,Me,H,OMe], [ZF104;H,Me,H,F], [ZF1 05;H,Me,H,Cl], [ZF106;H,OMe,Me,H], [ZF107;H,OMe,OMe,H], [ZF108;H,OMe,F,H], [ZF109;H,OMe,Cl,H], [ZF110;H,OMe,H,Me], [ZF111;H, OMe,H,OMe], [ZF112;H,OMe,H,F], [ZF113;H,OMe,H,Cl], [ZF114;H,F, Me,H], [ZF115;H,F,OMe,H], [ZF116;H,F,F,H], [ZF117;H,F,Cl,H], [Z F118;H,F,H,Me], [ZF119;H,F,H,OMe], [ZF120;H,F,H,F], [ZF121;H,F,H,Cl], [ZF122;H,Cl,Me,H], [ZF123;H,Cl,OMe,H], [ZF124;H,Cl,F,H], [ZF125;H,Cl,Cl,H], [ZF126;H,Cl,H,Me], [ZF127;H,Cl,H,OMe], [Z F128;H,Cl,H,F], [ZF129;H,Cl,H,Cl], [ZF130;H,H,Me,Me], [ZF131;H,H,Me,OMe], [ZF132;H,H,Me,F], [ZF133;H,H,Me,Cl], [ZF134;H,H,OM e,Me], [ZF135;H,H,OMe,OMe], [ZF136;H,H,OMe,F], [ZF137;H,H,OMe, Cl], [ZF138;H,H,F,Me], [ZF139;H,H,F,OMe], [ZF140;H,H,F,F], [ZF1 41;H,H,F,Cl], [ZF142;H,H,Cl,Me], [ZF143;H,H,Cl,OMe], [ZF144;H, H,Cl,F], [ZF145;H,H,Cl,Cl]

A compound according to Compound (1L) wherein R¹ represents a chlorine atom, Q represents Q1, and a combination of R^{X3}, R^{X4}, R^{X5} and R^{X6} represents any combination described in Combination F (hereinafter, referred to as Compound group SX972).

A compound according to Compound (1L) wherein R¹ represents an ethyl group, Q represents Q1, and a combination of R^{X3}, R^{X4}, R^{X5} and R^{X6} represents any combination described in Combination F (hereinafter, referred to as Compound group SX973).

A compound according to Compound (1L) wherein R¹ represents a fluorine atom, Q represents Q1, and a combination of R^{X3}, R^{X4}, R^{X5} and R^{X6} represents any combination described in Combination F (hereinafter, referred to as Compound group SX974).

A compound according to Compound (1L) wherein R¹ represents a cyclopropyl group, Q represents Q1, and a combination of R^{X3}, R^{X4}, R^{X5} and R^{X6} represents any combination described in Combination F (hereinafter, referred to as Compound group SX975).

A compound according to Compound (1L) wherein R¹ represents a methyl group, Q represents Q2, L¹ represents an oxygen atom, and a combination of R^{X3}, R^{X4}, R^{X5} and R^{X6} represents any combination described in Combination F (hereinafter, referred to as Compound group SX976).

A compound according to Compound (1L) wherein R¹ represents a chlorine atom, Q represents Q2, L¹ represents an oxygen atom, and a combination of R^{X3}, R^{X4}, R^{X5} and R^{X6} represents any combination described in Combination F (hereinafter, referred to as Compound group SX977).

A compound according to Compound (1L) wherein R¹ represents a methyl group, Q represents Q2, L¹ represents NH, and a combination of R^{X3}, R^{X4}, R^{X5} and R^{X6} represents any combination described in Combination F (hereinafter, referred to as Compound group SX978).

A compound according to Compound (1L) wherein R¹ represents a chlorine atom, Q represents Q2, L¹ represents NH, and a combination of R^{X3}, R^{X4}, R^{X5} and R^{X6} represents any combination described in Combination F (hereinafter, referred to as Compound group SX979).

A compound according to Compound (1L) wherein R¹ represents a methyl group, Q represents Q3, R³ represents a methoxy group, and a combination of R^{X3}, R^{X4}, R^{X5} and R^{X6} represents any combination described in Combination F (hereinafter, referred to as Compound group SX980).

A compound according to Compound (1L) wherein R¹ represents a chlorine atom, Q represents Q3, R³ represents a methoxy group, and a combination of R^{X3}, R^{X4}, R^{X5} and R^{X6} represents any combination described in Combination F (hereinafter, referred to as Compound group SX981).

A compound according to Compound (1L) wherein R¹ represents a methyl group, Q represents Q4, and a combination of R^{X3}, R^{X4}, R^{X5} and R^{X6} represents any combination described in Combination F (hereinafter, referred to as Compound group SX982).

A compound according to Compound (1L) wherein R¹ represents a chlorine atom, Q represents Q4, and a combination of R^{X3}, R^{X4}, R^{X5} and R^{X6} represents any combination described in Combination F (hereinafter, referred to as Compound group SX983).

A compound according to Compound (1L) wherein R¹ represents a methyl group, Q represents Q5, and a combination of R^{X3}, R^{X4}, R^{X5} and R^{X6} represents any combination described in Combination F (hereinafter, referred to as Compound group SX984).

A compound according to Compound (1L) wherein R¹ represents a chlorine atom, Q represents Q5, and a combination of R^{X3}, R^{X4}, R^{X5} and R^{X6} represents any combination described in Combination F (hereinafter, referred to as Compound group SX985).

A compound according to Compound (1L) wherein R¹ represents a methyl group, Q represents Q6, and a combination of R^{X3}, R^{X4}, R^{X5} and R^{X6} represents any combination described in Combination F (hereinafter, referred to as Compound group SX986).

A compound according to Compound (1L) wherein R¹ represents a chlorine atom, Q represents Q6, and a combination of R^{X3}, R^{X4}, R^{X5} and R^{X6} represents any combination described in Combination F (hereinafter, referred to as Compound group SX987).

A compound according to Compound (1L) wherein R¹ represents a methyl group, Q represents Q7, L² represents an oxygen atom, and a combination of R^{X3}, R^{X4}, R^{X5} and R^{X6} represents any combination described in Combination F (hereinafter, referred to as Compound group SX988).

A compound according to Compound (1L) wherein R¹ represents a chlorine atom, Q represents Q7, L² represents an oxygen atom, and a combination of R^{X3}, R^{X4}, R^{X5} and R^{X6} represents any combination described in Combination F (hereinafter, referred to as Compound group SX989).

A compound according to Compound (1L) wherein R¹ represents a methyl group, Q represents Q7, L² represents NH, and a combination of R^{X3}, R^{X4}, R^{X5} and R^{X6} represents any combination described in Combination F (hereinafter, referred to as Compound group SX990).

A compound according to Compound (1L) wherein R¹ represents a chlorine atom, Q represents Q7, L² represents NH, and a combination of R^{X3}, R^{X4}, R^{X5} and R^{X6} represents any combination described in Combination F (hereinafter, referred to as Compound group SX991).

A compound according to Compound (1L) wherein R¹ represents a methyl group, Q represents Q8A, and a combination of R^{X3}, R^{X4}, R^{X5} and R^{X6} represents any combination described in Combination F (hereinafter, referred to as Compound group SX992).

A compound according to Compound (1L) wherein R¹ represents a chlorine atom, Q represents Q8A, and a combination of R^{X3}, R^{X4}, R^{X5} and R^{X6} represents any combination described in Combination F (hereinafter, referred to as Compound group SX993).

A compound according to Compound (1L) wherein R¹ represents a methyl group, Q represents Q8D, and a combination of R^{X3}, R^{X4}, R^{X5} and R^{X6} represents any combination described in Combination F (hereinafter, referred to as Compound group SX994).

A compound according to Compound (1L) wherein R¹ represents a chlorine atom, Q represents Q8D, and a combination of R^{X3}, R^{X4}, R^{X5} and R^{X6} represents any combination described in Combination F (hereinafter, referred to as Compound group SX995).

A compound according to Compound (1L) wherein R¹ represents a methyl group, Q represents Q8J, and a combination of R^{X3}, R^{X4}, R^{X5} and R^{X6} represents any combination described in Combination F (hereinafter, referred to as Compound group SX996).

A compound according to Compound (1L) wherein R¹ represents a chlorine atom, Q represents Q8J, and a combination of R^{X3}, R^{X4}, R^{X5} and R^{X6} represents any combination described in Combination F (hereinafter, referred to as Compound group SX997).

A compound represented by formula (10) (hereinafter, referred to as Compound (1O)):

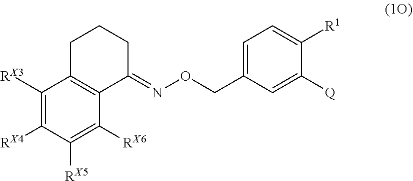

wherein R¹ represents a methyl group, Q represents Q1, and a combination of R^{X3}, R^{X4}, R^{X5} and R^{X6} represents any combination described in Combination F (hereinafter, referred to as Compound group SX998).

A compound according to Compound (1O) wherein R¹ represents a chlorine atom, Q represents Q1, and a combination of R^{X3}, R^{X4}, R^{X5} and R^{X6} represents any combination described in Combination F (hereinafter, referred to as Compound group SX999).

A compound according to Compound (1O) wherein R¹ represents an ethyl group, Q represents Q1, and a combination of R^{X3}, R^{X4}, R^{X5} and R^{X6} represents any combination described in Combination F (hereinafter, referred to as Compound group SX1000).

A compound according to Compound (1O) wherein R¹ represents a fluorine atom, Q represents Q1, and a combination of R^{X3}, R^{X4}, R^{X5} and R^{X6} represents any combination described in Combination F (hereinafter, referred to as Compound group SX1001).

A compound according to Compound (1O) wherein R¹ represents a cyclopropyl group, Q represents Q1, and a combination of R^{X3}, R^{X4}, R^{X5} and R^{X6} represents any combination described in Combination F (hereinafter, referred to as Compound group SX1002).

A compound according to Compound (1O) wherein R¹ represents a methyl group, Q represents Q2, L¹ represents an oxygen atom, and a combination of R^{X3}, R^{X4}, R^{X5} and R^{X6} represents any combination described in Combination F (hereinafter, referred to as Compound group SX1003).

A compound according to Compound (1O) wherein R¹ represents a chlorine atom, Q represents Q2, L¹ represents an oxygen atom, and a combination of R^{X3}, R^{X4}, R^{X5} and R^{X6} represents any combination described in Combination F (hereinafter, referred to as Compound group SX1004).

A compound according to Compound (1O) wherein R¹ represents a methyl group, Q represents Q2, L¹ represents NH, and a combination of R^{X3}, R^{X4}, R^{X5} and R^{X6} represents any combination described in Combination F (hereinafter, referred to as Compound group SX1005).

A compound according to Compound (1O) wherein R¹ represents a chlorine atom, Q represents Q2, L¹ represents NH, and a combination of R^{X3}, R^{X4}, R^{X5} and R^{X6} represents any combination described in Combination F (hereinafter, referred to as Compound group SX1006).

A compound according to Compound (1O) wherein R¹ represents a methyl group, Q represents Q3, R³ represents a methoxy group, and a combination of R^{X3}, R^{X4}, R^{X5} and R^{X6} represents any combination described in Combination F (hereinafter, referred to as Compound group SX1007).

A compound according to Compound (1O) wherein R¹ represents a chlorine atom, Q represents Q3, R³ represents a methoxy group, and a combination of R^{X3}, R^{X4}, R^{X5} and R^{X6} represents any combination described in Combination F (hereinafter, referred to as Compound group SX1008).

A compound according to Compound (1O) wherein R¹ represents a methyl group, Q represents Q4, and a combination of R^{X3}, R^{X4}, R^{X5} and R^{X6} represents any combination described in Combination F (hereinafter, referred to as Compound group SX1009).

A compound according to Compound (1O) wherein R¹ represents a chlorine atom, Q represents Q4, and a combination of R^{X3}, R^{X4}, R^{X5} and R^{X6} represents any combination described in Combination F (hereinafter, referred to as Compound group SX1010).

A compound according to Compound (1O) wherein R¹ represents a methyl group, Q represents Q5, and a combination of R^{X3}, R^{X4}, R^{X5} and R^{X6} represents any combination described in Combination F (hereinafter, referred to as Compound group SX1011).

A compound according to Compound (1O) wherein R¹ represents a chlorine atom, Q represents Q5, and a combination of R^{X3}, R^{X4}, R^{X5} and R^{X6} represents any combination described in Combination F (hereinafter, referred to as Compound group SX1012).

A compound according to Compound (1O) wherein R¹ represents a methyl group, Q represents Q6, and a combination of R^{X3}, R^{X4}, R^{X5} and R^{X6} represents any combination described in Combination F (hereinafter, referred to as Compound group SX1013).

A compound according to Compound (1O) wherein R¹ represents a chlorine atom, Q represents Q6, and a combination of R^{X3}, R^{X4}, R^{X5} and R^{X6} represents any combination described in Combination F (hereinafter, referred to as Compound group SX1014).

A compound according to Compound (1O) wherein R¹ represents a methyl group, Q represents Q7, L² represents an oxygen atom, and a combination of R^{X3}, R^{X4}, R^{X5} and R^{X6} represents any combination described in Combination F (hereinafter, referred to as Compound group SX1015).

A compound according to Compound (1O) wherein R¹ represents a chlorine atom, Q represents Q7, L² represents an oxygen atom, and a combination of R^{X3}, R^{X4}, R^{X5} and R^{X6} represents any combination described in Combination F (hereinafter, referred to as Compound group SX1016).

A compound according to Compound (1O) wherein R¹ represents a methyl group, Q represents Q7, L² represents NH, and a combination of R^{X3}, R^{X4}, R^{X5} and R^{X6} represents any combination described in Combination F (hereinafter, referred to as Compound group SX1017).

A compound according to Compound (1O) wherein R¹ represents a chlorine atom, Q represents Q7, L² represents NH, and a combination of R^{X3}, R^{X4}, R^{X5} and R^{X6} represents any combination described in Combination F (hereinafter, referred to as Compound group SX1018).

A compound according to Compound (1O) wherein R¹ represents a methyl group, Q represents Q8A, and a combination of R^{X3}, R^{X4}, R^{X5} and R^{X6} represents any combination described in Combination F (hereinafter, referred to as Compound group SX1019).

A compound according to Compound (1O) wherein R¹ represents a chlorine atom, Q represents Q8A, and a combination of R^{X3}, R^{X4}, R^{X5} and R^{X6} represents any combination described in Combination F (hereinafter, referred to as Compound group SX1020).

A compound according to Compound (1O) wherein R¹ represents a methyl group, Q represents Q8D, and a combination of R^{X3}, R^{X4}, R^{X5} and R^{X6} represents any combination described in Combination F (hereinafter, referred to as Compound group SX1021).

A compound according to Compound (1O) wherein R¹ represents a chlorine atom, Q represents Q8D, and a combination of R^{X3}, R^{X4}, R^{X5} and R^{X6} represents any combination described in Combination F (hereinafter, referred to as Compound group SX1022).

A compound according to Compound (1O) wherein R¹ represents a methyl group, Q represents Q8J, and a combination of R^{X3}, R^{X4}, R^{X5} and R^{X6} represents any combination described in Combination F (hereinafter, referred to as Compound group SX1023).

A compound according to Compound (1O) wherein R¹ represents a chlorine atom, Q represents Q8J, and a combination of R^{X3}, R^{X4}, R^{X5} and R^{X6} represents any combination described in Combination F (hereinafter, referred to as Compound group SX1024).

A compound represented by formula (1Q) (hereinafter, referred to as Compound (1Q)):

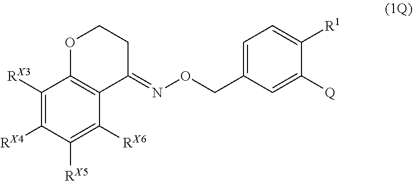

wherein R¹ represents a methyl group, Q represents Q1, and a combination of R^{X3}, R^{X4}, R^{X5} and R^{X6} represents any combination described in Combination F (hereinafter, referred to as Compound group SX1025).

A compound according to Compound (1Q) wherein R¹ represents a chlorine atom, Q represents Q1, and a combination of R^{X3}, R^{X4}, R^{X5} and R^{X6} represents any combination described in Combination F (hereinafter, referred to as Compound group SX1026).

A compound according to Compound (1Q) wherein R¹ represents an ethyl group, Q represents Q1, and a combination of R^{X3}, R^{X4}, R^{X5} and R^{X6} represents any combination described in Combination F (hereinafter, referred to as Compound group SX1027).

A compound according to Compound (1Q) wherein R¹ represents a fluorine atom, Q represents Q1, and a combination of R^{X3}, R^{X4}, R^{X5} and R^{X6} represents any combination described in Combination F (hereinafter, referred to as Compound group SX1028).

A compound according to Compound (1Q) wherein R¹ represents a cyclopropyl group, Q represents Q1, and a combination of R^{X3}, R^{X4}, R^{X5} and R^{X6} represents any combination described in Combination F (hereinafter, referred to as Compound group SX1029).

A compound according to Compound (1Q) wherein R¹ represents a methyl group, Q represents Q2, L¹ represents an oxygen atom, and a combination of R^{X3}, R^{X4}, R^{X5} and R^{X6} represents any combination described in Combination F (hereinafter, referred to as Compound group SX1030).

A compound according to Compound (1Q) wherein R¹ represents a chlorine atom, Q represents Q2, L¹ represents an oxygen atom, and a combination of R^{X3}, R^{X4}, R^{X5} and R^{X6} represents any combination described in Combination F (hereinafter, referred to as Compound group SX1031).

A compound according to Compound (1Q) wherein R¹ represents a methyl group, Q represents Q2, L¹ represents NH, and a combination of R^{X3}, R^{X4}, R^{X5} and R^{X6} represents any combination described in Combination F (hereinafter, referred to as Compound group SX1032).

A compound according to Compound (1Q) wherein R¹ represents a chlorine atom, Q represents Q2, L¹ represents NH, and a combination of R^{X3}, R^{X4}, R^{X5} and R^{X6} represents any combination described in Combination F (hereinafter, referred to as Compound group SX1033).

A compound according to Compound (1Q) wherein R¹ represents a methyl group, Q represents Q3, R³ represents a methoxy group, and a combination of R^{X3}, R^{X4}, R^{X5} and R^{X6} represents any combination described in Combination F (hereinafter, referred to as Compound group SX1034).

A compound according to Compound (1Q) wherein R¹ represents a chlorine atom, Q represents Q3, R³ represents a methoxy group, and a combination of R^{X3}, R^{X4}, R^{X5} and R^{X6} represents any combination described in Combination F (hereinafter, referred to as Compound group SX1035).

A compound according to Compound (1Q) wherein R¹ represents a methyl group, Q represents Q4, and a combination of R^{X3}, R^{X4}, R^{X5} and R^{X6} represents any combination described in Combination F (hereinafter, referred to as Compound group SX1036).

A compound according to Compound (1Q) wherein R¹ represents a chlorine atom, Q represents Q4, and a combination of R^{X3}, R^{X4}, R^{X5} and R^{X6} represents any combination described in Combination F (hereinafter, referred to as Compound group SX1037).

A compound according to Compound (1Q) wherein R¹ represents a methyl group, Q represents Q5, and a combination of R^{X3}, R^{X4}, R^{X5} and R^{X6} represents any combination described in Combination F (hereinafter, referred to as Compound group SX1038).

A compound according to Compound (1Q) wherein R¹ represents a chlorine atom, Q represents Q5, and a combination of R^{X3}, R^{X4}, R^{X5} and R^{X6} represents any combination described in Combination F (hereinafter, referred to as Compound group SX1039).

A compound according to Compound (1Q) wherein R¹ represents a methyl group, Q represents Q6, and a combination of R^{X3}, R^{X4}, R^{X5} and R^{X6} represents any combination described in Combination F (hereinafter, referred to as Compound group SX1040).

A compound according to Compound (1Q) wherein R¹ represents a chlorine atom, Q represents Q6, and a combination of R^{X3}, R^{X4}, R^{X5} and R^{X6} represents any combination described in Combination F (hereinafter, referred to as Compound group SX1041).

A compound according to Compound (1Q) wherein R¹ represents a methyl group, Q represents Q7, L² represents an oxygen atom, and a combination of R^{X3}, R^{X4}, R^{X5} and R^{X6} represents any combination described in Combination F (hereinafter, referred to as Compound group SX1042).

A compound according to Compound (1Q) wherein R¹ represents a chlorine atom, Q represents Q7, L² represents an oxygen atom, and a combination of R^{X3}, R^{X4}, R^{X5} and R^{X6} represents any combination described in Combination F (hereinafter, referred to as Compound group SX1043).

A compound according to Compound (1Q) wherein R¹ represents a methyl group, Q represents Q7, L² represents NH, and a combination of R^{X3}, R^{X4}, R^{X5} and R^{X6} represents any combination described in Combination F (hereinafter, referred to as Compound group SX1044).

A compound according to Compound (1Q) wherein R¹ represents a chlorine atom, Q represents Q7, L² represents NH, and a combination of R^{X3}, R^{X4}, R^{X5} and R^{X6} represents any combination described in Combination F (hereinafter, referred to as Compound group SX1045).

A compound according to Compound (1Q) wherein R¹ represents a methyl group, Q represents Q8A, and a combination of R^{X3}, R^{X4}, R^{X5} and R^{X6} represents any combination described in Combination F (hereinafter, referred to as Compound group SX1046).

A compound according to Compound (1Q) wherein R¹ represents a chlorine atom, Q represents Q8A, and a combination of R^{X3}, R^{X4}, R^{X5} and R^{X6} represents any combination described in Combination F (hereinafter, referred to as Compound group SX1047).

A compound according to Compound (1Q) wherein R¹ represents a methyl group, Q represents Q8D, and a combination of R^{X3}, R^{X4}, R^{X5} and R^{X6} represents any combination described in Combination F (hereinafter, referred to as Compound group SX1048).

A compound according to Compound (1Q) wherein R¹ represents a chlorine atom, Q represents Q8D, and a combination of R^{X3}, R^{X4}, R^{X5} and R^{X6} represents any combination described in Combination F (hereinafter, referred to as Compound group SX1049).

A compound according to Compound (1Q) wherein R¹ represents a methyl group, Q represents Q8J, and a combination of R^{X3}, R^{X4}, R^{X5} and R^{X6} represents any combination described in Combination F (hereinafter, referred to as Compound group SX1050).

A compound according to Compound (1Q) wherein R¹ represents a chlorine atom, Q represents Q8J, and a combination of R^{X3}, R^{X4}, R^{X5} and R^{X6} represents any combination described in Combination F (hereinafter, referred to as Compound group SX1051).

A compound represented by formula (1R) (hereinafter, referred to as Compound (1R)):

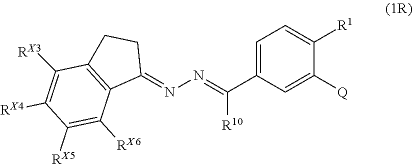

wherein R¹ represents a methyl group, Q represents Q1, R¹⁰ represents a methyl group, and a combination of R^{X3}, R^{X4}, R^{X5} and R^{X6} represents any combination described in Combination F (hereinafter, referred to as Compound group SX1052).

A compound according to Compound (1R) wherein R¹ represents a chlorine atom, Q represents Q1, R¹⁰ represents a methyl group, and a combination of R^{X3}, R^{X4}, R^{X5} and R^{X6} represents any combination described in Combination F (hereinafter, referred to as Compound group SX1053).

A compound according to Compound (1R) wherein R¹ represents an ethyl group, Q represents Q1, R¹⁰ represents a methyl group, and a combination of R^{X3}, R^{X4}, R^{X5} and R^{X6} represents any combination described in Combination F (hereinafter, referred to as Compound group SX1054).

A compound according to Compound (1R) wherein R¹ represents a fluorine atom, Q represents Q1, R¹⁰ represents a methyl group, and a combination of R^{X3}, R^{X4}, R^{X5} and R^{X6} represents any combination described in Combination F (hereinafter, referred to as Compound group SX1055).

A compound according to Compound (1R) wherein R¹ represents a cyclopropyl group, Q represents Q1, R¹⁰ represents a methyl group, and a combination of R^{X3}, R^{X4}, R^{X5} and R^{X6} represents any combination described in Combination F (hereinafter, referred to as Compound group SX1056).

A compound according to Compound (1R) wherein R¹ represents a methyl group, Q represents Q2, L¹ represents an oxygen atom, R¹⁰ represents a methyl group, and a combination of R^{X3}, R^{X4}, R^{X5} and R^{X6} represents any combination described in Combination F (hereinafter, referred to as Compound group SX1057).

A compound according to Compound (1R) wherein R¹ represents a chlorine atom, Q represents Q2, L¹ represents an oxygen atom, R¹⁰ represents a methyl group, and a combination of R^{X3}, R^{X4}, R^{X5} and R^{X6} represents any combination described in Combination F (hereinafter, referred to as Compound group SX1058).

A compound according to Compound (1R) wherein R¹ represents a methyl group, Q represents Q2, L¹ represents NH, R¹⁰ represents a methyl group, and a combination of R^{X3}, R^{X4}, R^{X5} and R^{X6} represents any combination described in Combination F (hereinafter, referred to as Compound group SX1059).

A compound according to Compound (1R) wherein R¹ represents a chlorine atom, Q represents Q2, L¹ represents NH, R¹⁰ represents a methyl group, and a combination of R^{X3}, R^{X4}, R^{X5} and R^{X6} represents any combination described in Combination F (hereinafter, referred to as Compound group SX1060).

A compound according to Compound (1R) wherein R¹ represents a methyl group, Q represents Q3, R³ represents a methoxy group, R¹⁰ represents a methyl group, and a combination of R^{X3}, R^{X4}, R^{X5} and R^{X6} represents any combination described in Combination F (hereinafter, referred to as Compound group SX1061).

A compound according to Compound (1R) wherein R¹ represents a chlorine atom, Q represents Q3, R³ represents a methoxy group, R¹⁰ represents a methyl group, and a combination of R^{X3}, R^{X4}, R^{X5} and R^{X6} represents any combination described in Combination F (hereinafter, referred to as Compound group SX1062).

A compound according to Compound (1R) wherein R¹ represents a methyl group, Q represents Q4, R¹⁰ represents a methyl group, and a combination of R^{X3}, R^{X4}, R^{X5} and R^{X6} represents any combination described in Combination F (hereinafter, referred to as Compound group SX1063).

A compound according to Compound (1R) wherein R¹ represents a chlorine atom, Q represents Q4, R¹⁰ represents a methyl group, and a combination of R^{X3}, R^{X4}, R^{X5} and R^{X6} represents any combination described in Combination F (hereinafter, referred to as Compound group SX1064).

A compound according to Compound (1R) wherein R¹ represents a methyl group, Q represents Q5, R¹⁰ represents a methyl group, and a combination of R^{X3}, R^{X4}, R^{X5} and R^{X6} represents any combination described in Combination F (hereinafter, referred to as Compound group SX1065).

A compound according to Compound (1R) wherein R¹ represents a chlorine atom, Q represents Q5, R¹⁰ represents a methyl group, and a combination of R^{X3}, R^{X4}, R^{X5} and R^{X6} represents any combination described in Combination F (hereinafter, referred to as Compound group SX1066).

A compound according to Compound (1R) wherein R¹ represents a methyl group, Q represents Q6, R¹⁰ represents a methyl group, and a combination of R^{X3}, R^{X4}, R^{X5} and R^{X6} represents any combination described in Combination F (hereinafter, referred to as Compound group SX1067).

A compound according to Compound (1R) wherein R¹ represents a chlorine atom, Q represents Q6, R¹⁰ represents a methyl group, and a combination of R^{X3}, R^{X4}, R^{X5} and R^{X6} represents any combination described in Combination F (hereinafter, referred to as Compound group SX1068).

A compound according to Compound (1R) wherein R¹ represents a methyl group, Q represents Q7, L² represents an oxygen atom, R¹⁰ represents a methyl group, and a combination of R^{X3}, R^{X4}, R^{X5} and R^{X6} represents any combination described in Combination F (hereinafter, referred to as Compound group SX1069).

A compound according to Compound (1R) wherein R¹ represents a chlorine atom, Q represents Q7, L² represents an oxygen atom, R¹⁰ represents a methyl group, and a combination of R^{X3}, R^{X4}, R^{X5} and R^{X6} represents any combination described in Combination F (hereinafter, referred to as Compound group SX1070).

A compound according to Compound (1R) wherein R¹ represents a methyl group, Q represents Q7, L² represents NH, R¹⁰ represents a methyl group, and a combination of R^{X3}, R^{X4}, R^{X5} and R^{X6} represents any combination described in Combination F (hereinafter, referred to as Compound group SX1071).

A compound according to Compound (1R) wherein R¹ represents a chlorine atom, Q represents Q7, L² represents NH, R¹⁰ represents a methyl group, and a combination of R^{X3}, R^{X4}, R^{X5} and R^{X6} represents any combination described in Combination F (hereinafter, referred to as Compound group SX1072).

A compound according to Compound (1R) wherein R¹ represents a methyl group, Q represents Q8A, R¹⁰ represents a methyl group, and a combination of R^{X3}, R^{X4}, R^{X5} and R^{X6} represents any combination described in Combination F (hereinafter, referred to as Compound group SX1073).

A compound according to Compound (1R) wherein R¹ represents a chlorine atom, Q represents Q8A, R¹⁰ represents a methyl group, and a combination of R^{X3}, R^{X4}, R^{X5} and R^{X6} represents any combination described in Combination F (hereinafter, referred to as Compound group SX1074).

A compound according to Compound (1R) wherein R¹ represents a methyl group, Q represents Q8D, R¹⁰ represents a methyl group, and a combination of R^{X3}, R^{X4}, R^{X5} and R^{X6} represents any combination described in Combination F (hereinafter, referred to as Compound group SX1075).

A compound according to Compound (1R) wherein R¹ represents a chlorine atom, Q represents Q8D, R¹⁰ represents a methyl group, and a combination of R^{X3}, R^{X4}, R^{X5} and R^{X6} represents any combination described in Combination F (hereinafter, referred to as Compound group SX1076).

A compound according to Compound (1R) wherein R¹ represents a methyl group, Q represents Q8J, R¹⁰ represents a methyl group, and a combination of R^{X3}, R^{X4}, R^{X5} and R^{X6} represents any combination described in Combination F (hereinafter, referred to as Compound group SX1077).

A compound according to Compound (1R) wherein R¹ represents a chlorine atom, Q represents Q8J, R¹⁰ represents a methyl group, and a combination of R^{X3}, R^{X4}, R^{X5} and R^{X6} represents any combination described in Combination F (hereinafter, referred to as Compound group SX1078).

A compound according to Compound (1R) wherein R¹ represents a methyl group, Q represents Q1, R¹⁰ represents an ethyl group, and a combination of R^{X3}, R^{X4}, R^{X5} and R^{X6} represents any combination described in Combination F (hereinafter, referred to as Compound group SX1079).

A compound according to Compound (1R) wherein R¹ represents a chlorine atom, Q represents Q1, R¹⁰ represents an ethyl group, and a combination of R^{X3}, R^{X4}, R^{X5} and R^{X6} represents any combination described in Combination F (hereinafter, referred to as Compound group SX1080).

A compound according to Compound (1R) wherein R¹ represents an ethyl group, Q represents Q1, R¹⁰ represents an ethyl group, and a combination of R^{X3}, R^{X4}, R^{X5} and R^{X6} represents any combination described in Combination F (hereinafter, referred to as Compound group SX1081).

A compound according to Compound (1R) wherein R¹ represents a fluorine atom, Q represents Q1, R¹⁰ represents an ethyl group, and a combination of R^{X3}, R^{X4}, R^{X5} and R^{X6} represents any combination described in Combination F (hereinafter, referred to as Compound group SX1082).

A compound according to Compound (1R) wherein R¹ represents a cyclopropyl group, Q represents Q1, R¹⁰ represents an ethyl group, and a combination of R^{X3}, R^{X4}, R^{X5} and R^{X6} represents any combination described in Combination F (hereinafter, referred to as Compound group SX1083).

A compound according to Compound (1R) wherein R¹ represents a methyl group, Q represents Q2, L¹ represents an oxygen atom, R¹⁰ represents an ethyl group, and a combination of R^{X3}, R^{X4}, R^{X5} and R^{X6} represents any combination described in Combination F (hereinafter, referred to as Compound group SX1084).

A compound according to Compound (1R) wherein R¹ represents a chlorine atom, Q represents Q2, L¹ represents an oxygen atom, R¹⁰ represents an ethyl group, and a combination of R^{X3}, R^{X4}, R^{X5} and R^{X6} represents any combination described in Combination F (hereinafter, referred to as Compound group SX1085).

A compound according to Compound (1R) wherein R¹ represents a methyl group, Q represents Q2, L¹ represents NH, R¹⁰ represents an ethyl group, and a combination of R^{X3}, R^{X4}, R^{X5} and R^{X6} represents any combination described in Combination F (hereinafter, referred to as Compound group SX1086).

A compound according to Compound (1R) wherein R¹ represents a chlorine atom, Q represents Q2, L¹ represents NH, R¹⁰ represents an ethyl group, and a combination of R^{X3}, R^{X4}, R^{X5} and R^{X6} represents any combination described in Combination F (hereinafter, referred to as Compound group SX1087).

A compound according to Compound (1R) wherein R¹ represents a methyl group, Q represents Q3, R³ represents a methoxy group, R¹⁰ represents an ethyl group, and a combination of R^{X3}, R^{X4}, R^{X5} and R^{X6} represents any combination described in Combination F (hereinafter, referred to as Compound group SX1088).

A compound according to Compound (1R) wherein R¹ represents a chlorine atom, Q represents Q3, R³ represents a methoxy group, R¹⁰ represents an ethyl group, and a combination of R^{X3}, R^{X4}, R^{X5} and R^{X6} represents any combination described in Combination F (hereinafter, referred to as Compound group SX1089).

A compound according to Compound (1R) wherein R¹ represents a methyl group, Q represents Q4, R¹⁰ represents an ethyl group, and a combination of R^{X3}, R^{X4}, R^{X5} and R^{X6} represents any combination described in Combination F (hereinafter, referred to as Compound group SX1090).

A compound according to Compound (1R) wherein R¹ represents a chlorine atom, Q represents Q4, R¹⁰ represents an ethyl group, and a combination of R^{X3}, R^{X4}, R^{X5} and R^{X6} represents any combination described in Combination F (hereinafter, referred to as Compound group SX1091).

A compound according to Compound (1R) wherein R¹ represents a methyl group, Q represents Q5, R¹⁰ represents an ethyl group, and a combination of R^{X3}, R^{X4}, R^{X5} and R^{X6} represents any combination described in Combination F (hereinafter, referred to as Compound group SX1092).

A compound according to Compound (1R) wherein R¹ represents a chlorine atom, Q represents Q5, R¹⁰ represents an ethyl group, and a combination of R^{X3}, R^{X4}, R^{X5} and R^{X6} represents any combination described in Combination F (hereinafter, referred to as Compound group SX1093).

A compound according to Compound (1R) wherein R¹ represents a methyl group, Q represents Q6, R¹⁰ represents an ethyl group, and a combination of R^{X3}, R^{X4}, R^{X5} and R^{X6} represents any combination described in Combination F (hereinafter, referred to as Compound group SX1094).

A compound according to Compound (1R) wherein R¹ represents a chlorine atom, Q represents Q6, R¹⁰ represents an ethyl group, and a combination of R^{X3}, R^{X4}, R^{X5} and R^{X6} represents any combination described in Combination F (hereinafter, referred to as Compound group SX1095).

A compound according to Compound (1R) wherein R¹ represents a methyl group, Q represents Q7, L² represents an oxygen atom, R¹⁰ represents an ethyl group, and a combination of R^{X3}, R^{X4}, R^{X5} and R^{X6} represents any combination described in Combination F (hereinafter, referred to as Compound group SX1096).

A compound according to Compound (1R) wherein R¹ represents a chlorine atom, Q represents Q7, L² represents an oxygen atom, R¹⁰ represents an ethyl group, and a combination of R^{X3}, R^{X4}, R^{X5} and R^{X6} represents any combination described in Combination F (hereinafter, referred to as Compound group SX1097).

A compound according to Compound (1R) wherein R¹ represents a methyl group, Q represents Q7, L² represents NH, R¹⁰ represents an ethyl group, and a combination of R^{X3}, R^{X4}, R^{X5} and R^{X6} represents any combination described in Combination F (hereinafter, referred to as Compound group SX1098).

A compound according to Compound (1R) wherein R¹ represents a chlorine atom, Q represents Q7, L² represents NH, R¹⁰ represents an ethyl group, and a combination of R^{X3}, R^{X4}, R^{X5} and R^{X6} represents any combination described in Combination F (hereinafter, referred to as Compound group SX1099).

A compound according to Compound (1R) wherein R¹ represents a methyl group, Q represents Q8A, R¹⁰ represents an ethyl group, and a combination of R^{X3}, R^{X4}, R^{X5} and R^{X6} represents any combination described in Combination F (hereinafter, referred to as Compound group SX1100).

A compound according to Compound (1R) wherein R¹ represents a chlorine atom, Q represents Q8A, R¹⁰ represents an ethyl group, and a combination of R^{X3}, R^{X4}, R^{X5} and R^{X6} represents any combination described in Combination F (hereinafter, referred to as Compound group SX1101).

A compound according to Compound (1R) wherein R¹ represents a methyl group, Q represents Q8D, R¹⁰ represents an ethyl group, and a combination of R^{X3}, R^{X4}, R^{X5} and R^{X6} represents any combination described in Combination F (hereinafter, referred to as Compound group SX1102).

A compound according to Compound (1R) wherein R¹ represents a chlorine atom, Q represents Q8D, R¹⁰ represents an ethyl group, and a combination of R^{X3}, R^{X4}, R^{X5} and R^{X6} represents any combination described in Combination F (hereinafter, referred to as Compound group SX1103).

A compound according to Compound (1R) wherein R¹ represents a methyl group, Q represents Q8J, R¹⁰ represents an ethyl group, and a combination of R^{X3}, R^{X4}, R^{X5} and R^{X6} represents any combination described in Combination F (hereinafter, referred to as Compound group SX1104).

A compound according to Compound (1R) wherein R¹ represents a chlorine atom, Q represents Q8J, R¹⁰ represents an ethyl group, and a combination of R^{X3}, R^{X4}, R^{X5} and R^{X6} represents any combination described in Combination F (hereinafter, referred to as Compound group SX1105).

A compound according to Compound (1R) wherein R¹ represents a methyl group, Q represents Q1, R¹⁰ represents a cyclopropyl group, and a combination of R^{X3}, R^{X4}, R^{X5} and R^{X6} represents any combination described in Combination F (hereinafter, referred to as Compound group SX1106).

A compound according to Compound (1R) wherein R¹ represents a chlorine atom, Q represents Q1, R¹⁰ represents a cyclopropyl group, and a combination of R^{X3}, R^{X4}, R^{X5} and R^{X6} represents any combination described in Combination F (hereinafter, referred to as Compound group SX1107).

A compound according to Compound (1R) wherein R¹ represents an ethyl group, Q represents Q1, R¹⁰ represents a cyclopropyl group, and a combination of R^{X3}, R^{X4}, R^{X5} and R^{X6} represents any combination described in Combination F (hereinafter, referred to as Compound group SX1108).

A compound according to Compound (1R) wherein R¹ represents a fluorine atom, Q represents Q1, R¹⁰ represents a cyclopropyl group, and a combination of R^{X3}, R^{X4}, R^{X5} and R^{X6} represents any combination described in Combination F (hereinafter, referred to as Compound group SX1109).

A compound according to Compound (1R) wherein R¹ represents a cyclopropyl group, Q represents Q1, R¹⁰ represents a cyclopropyl group, and a combination of R^{X3}, R^{X4}, R^{X5} and R^{X6} represents any combination described in Combination F (hereinafter, referred to as Compound group SX1110).

A compound according to Compound (1R) wherein R¹ represents a methyl group, Q represents Q2, L¹ represents an oxygen atom, R¹⁰ represents a cyclopropyl group, and a combination of R^{X3}, R^{X4}, R^{X5} and R^{X6} represents any combination described in Combination F (hereinafter, referred to as Compound group SX1111).

A compound according to Compound (1R) wherein R¹ represents a chlorine atom, Q represents Q2, L¹ represents an oxygen atom, R¹⁰ represents a cyclopropyl group, and a combination of R^{X3}, R^{X4}, R^{X5} and R^{X6} represents any combination described in Combination F (hereinafter, referred to as Compound group SX1112).

A compound according to Compound (1R) wherein R¹ represents a methyl group, Q represents Q2, L¹ represents NH, R¹⁰ represents a cyclopropyl group, and a combination of R^{X3}, R^{X4}, R^{X5} and R^{X6} represents any combination described in Combination F (hereinafter, referred to as Compound group SX1113).

A compound according to Compound (1R) wherein R¹ represents a chlorine atom, Q represents Q2, L¹ represents NH, R¹⁰ represents a cyclopropyl group, and a combination of R^{X3}, R^{X4}, R^{X5} and R^{X6} represents any combination described in Combination F (hereinafter, referred to as Compound group SX1114).

A compound according to Compound (1R) wherein R¹ represents a methyl group, Q represents Q3, R³ represents a methoxy group, R¹⁰ represents a cyclopropyl group, and a combination of R^{X3}, R^{X4}, R^{X5} and R^{X6} represents any combination described in Combination F (hereinafter, referred to as Compound group SX1115).

A compound according to Compound (1R) wherein R¹ represents a chlorine atom, Q represents Q3, R³ represents a methoxy group, R¹⁰ represents a cyclopropyl group, and a combination of R^{X3}, R^{X4}, R^{X5} and R^{X6} represents any combination described in Combination F (hereinafter, referred to as Compound group SX1116).

A compound according to Compound (1R) wherein R¹ represents a methyl group, Q represents Q4, R¹⁰ represents a cyclopropyl group, and a combination of R^{X3}, R^{X4}, R^{X5} and R^{X6} represents any combination described in Combination F (hereinafter, referred to as Compound group SX1117).

A compound according to Compound (1R) wherein R¹ represents a chlorine atom, Q represents Q4, R¹⁰ represents a cyclopropyl group, and a combination of R^{X3}, R^{X4}, R^{X5} and R^{X6} represents any combination described in Combination F (hereinafter, referred to as Compound group SX1118).

A compound according to Compound (1R) wherein R¹ represents a methyl group, Q represents Q5, R¹⁰ represents a cyclopropyl group, and a combination of R^{X3}, R^{X4}, R^{X5} and R^{X6} represents any combination described in Combination F (hereinafter, referred to as Compound group SX1119).

A compound according to Compound (1R) wherein R¹ represents a chlorine atom, Q represents Q5, R¹⁰ represents a cyclopropyl group, and a combination of R^{X3}, R^{X4}, R^{X5} and R^{X6} represents any combination described in Combination F (hereinafter, referred to as Compound group SX1120).

A compound according to Compound (1R) wherein R¹ represents a methyl group, Q represents Q6, R¹⁰ represents a cyclopropyl group, and a combination of R^{X3}, R^{X4}, R^{X5} and R^{X6} represents any combination described in Combination F (hereinafter, referred to as Compound group SX1121).

A compound according to Compound (1R) wherein R¹ represents a chlorine atom, Q represents Q6, R¹⁰ represents a cyclopropyl group, and a combination of R^{X3}, R^{X4}, R^{X5} and R^{X6} represents any combination described in Combination F (hereinafter, referred to as Compound group SX1122).

A compound according to Compound (1R) wherein R¹ represents a methyl group, Q represents Q7, L² represents an oxygen atom, R¹⁰ represents a cyclopropyl group, and a combination of R^{X3}, R^{X4}, R^{X5} and R^{X6} represents any combination described in Combination F (hereinafter, referred to as Compound group SX1123).

A compound according to Compound (1R) wherein R¹ represents a chlorine atom, Q represents Q7, L² represents an oxygen atom, R¹⁰ represents a cyclopropyl group, and a combination of R^{X3}, R^{X4}, R^{X5} and R^{X6} represents any combination described in Combination F (hereinafter, referred to as Compound group SX1124).

A compound according to Compound (1R) wherein R¹ represents a methyl group, Q represents Q7, L² represents NH, R¹⁰ represents a cyclopropyl group, and a combination of R^{X3}, R^{X4}, R^{X5} and R^{X6} represents any combination described in Combination F (hereinafter, referred to as Compound group SX1125).

A compound according to Compound (1R) wherein R¹ represents a chlorine atom, Q represents Q7, L² represents NH, R¹⁰ represents a cyclopropyl group, and a combination of R^{X3}, R^{X4}, R^{X5} and R^{X6} represents any combination described in Combination F (hereinafter, referred to as Compound group SX1126).

A compound according to Compound (1R) wherein R¹ represents a methyl group, Q represents Q8A, R¹⁰ represents a cyclopropyl group, and a combination of R^{X3}, R^{X4}, R^{X5} and R^{X6} represents any combination described in Combination F (hereinafter, referred to as Compound group SX1127).

A compound according to Compound (1R) wherein R¹ represents a chlorine atom, Q represents Q8A, R¹⁰ represents a cyclopropyl group, and a combination of R^{X3}, R^{X4}, R^{X5} and R^{X6} represents any combination described in Combination F (hereinafter, referred to as Compound group SX1128).

A compound according to Compound (1R) wherein R¹ represents a methyl group, Q represents Q8D, R¹⁰ represents a cyclopropyl group, and a combination of R^{X3}, R^{X4}, R^{X5} and R^{X6} represents any combination described in Combination F (hereinafter, referred to as Compound group SX1129).

A compound according to Compound (1R) wherein R¹ represents a chlorine atom, Q represents Q8D, R¹⁰ represents a cyclopropyl group, and a combination of R^{X3}, R^{X4}, R^{X5} and R^{X6} represents any combination described in Combination F (hereinafter, referred to as Compound group SX1130).

A compound according to Compound (1R) wherein R¹ represents a methyl group, Q represents Q8J, R¹⁰ represents a cyclopropyl group, and a combination of R^{X3}, R^{X4}, R^{X5} and R^{X6} represents any combination described in Combination F (hereinafter, referred to as Compound group SX1131).

A compound according to Compound (1R) wherein R¹ represents a chlorine atom, Q represents Q8J, R¹⁰ represents a cyclopropyl group, and a combination of R^{X3}, R^{X4}, R^{X5} and R^{X6} represents any combination described in Combination F (hereinafter, referred to as Compound group SX1132).

A compound represented by formula (1S) (hereinafter, referred to as Compound (1S)):

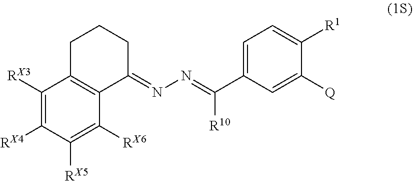

wherein R¹ represents a methyl group, Q represents Q1, R¹⁰ represents a methyl group, and a combination of R^{X3}, R^{X4}, R^{X5} and R^{X6} represents any combination described in Combination F (hereinafter, referred to as Compound group SX1133).

A compound according to Compound (1S) wherein R¹ represents a chlorine atom, Q represents Q1, R¹⁰ represents a methyl group, and a combination of R^{X3}, R^{X4}, R^{X5} and R^{X6} represents any combination described in Combination F (hereinafter, referred to as Compound group SX1134).

A compound according to Compound (1S) wherein R¹ represents an ethyl group, Q represents Q1, R¹⁰ represents a methyl group, and a combination of R^{X3}, R^{X4}, R^{X5} and R^{X6} represents any combination described in Combination F (hereinafter, referred to as Compound group SX1135).

A compound according to Compound (1S) wherein R¹ represents a fluorine atom, Q represents Q1, R¹⁰ represents a methyl group, and a combination of R^{X3}, R^{X4}, R^{X5} and R^{X6} represents any combination described in Combination F (hereinafter, referred to as Compound group SX1136).

A compound according to Compound (1S) wherein R¹ represents a cyclopropyl group, Q represents Q1, R¹⁰ represents a methyl group, and a combination of R^{X3}, R^{X4}, R^{X5} and R^{X6} represents any combination described in Combination F (hereinafter, referred to as Compound group SX1137).

A compound according to Compound (1S) wherein R¹ represents a methyl group, Q represents Q2, L¹ represents an oxygen atom, R¹⁰ represents a methyl group, and a combination of R^{X3}, R^{X4}, R^{X5} and R^{X6} represents any combination described in Combination F (hereinafter, referred to as Compound group SX1138).

A compound according to Compound (1S) wherein R¹ represents a chlorine atom, Q represents Q2, L¹ represents an oxygen atom, R¹⁰ represents a methyl group, and a combination of R^{X3}, R^{X4}, R^{X5} and R^{X6} represents any combination described in Combination F (hereinafter, referred to as Compound group SX1139).

A compound according to Compound (1S) wherein R¹ represents a methyl group, Q represents Q2, L¹ represents NH, R¹⁰ represents a methyl group, and a combination of R^{X3}, R^{X4}, R^{X5} and R^{X6} represents any combination described in Combination F (hereinafter, referred to as Compound group SX1140).

A compound according to Compound (1S) wherein R¹ represents a chlorine atom, Q represents Q2, L¹ represents NH, R¹⁰ represents a methyl group, and a combination of R^{X3}, R^{X4}, R^{X5} and R^{X6} represents any combination described in Combination F (hereinafter, referred to as Compound group SX1141).

A compound according to Compound (1S) wherein R¹ represents a methyl group, Q represents Q3, R³ represents a methoxy group, R¹⁰ represents a methyl group, and a combination of R^{X3}, R^{X4}, R^{X5} and R^{X6} represents any combination described in Combination F (hereinafter, referred to as Compound group SX1142).

A compound according to Compound (1S) wherein R¹ represents a chlorine atom, Q represents Q3, R³ represents a methoxy group, R¹⁰ represents a methyl group, and a combination of R^{X3}, R^{X4}, R^{X5} and R^{X6} represents any combination described in Combination F (hereinafter, referred to as Compound group SX1143).

A compound according to Compound (1S) wherein R¹ represents a methyl group, Q represents Q4, R¹⁰ represents a methyl group, and a combination of R^{X3}, R^{X4}, R^{X5} and R^{X6} represents any combination described in Combination F (hereinafter, referred to as Compound group SX1144).

A compound according to Compound (1S) wherein R¹ represents a chlorine atom, Q represents Q4, R¹⁰ represents a methyl group, and a combination of R^{X3}, R^{X4}, R^{X5} and R^{X6} represents any combination described in Combination F (hereinafter, referred to as Compound group SX1145).

A compound according to Compound (1S) wherein R¹ represents a methyl group, Q represents Q5, R¹⁰ represents a methyl group, and a combination of R^{X3}, R^{X4}, R^{X5} and R^{X6} represents any combination described in Combination F (hereinafter, referred to as Compound group SX1146).

A compound according to Compound (1S) wherein R¹ represents a chlorine atom, Q represents Q5, R¹⁰ represents a methyl group, and a combination of R^{X3}, R^{X4}, R^{X5} and R^{X6} represents any combination described in Combination F (hereinafter, referred to as Compound group SX1147).

A compound according to Compound (1S) wherein R¹ represents a methyl group, Q represents Q6, R¹⁰ represents a methyl group, and a combination of R^{X3}, R^{X4}, R^{X5} and R^{X6} represents any combination described in Combination F (hereinafter, referred to as Compound group SX1148).

A compound according to Compound (1S) wherein R¹ represents a chlorine atom, Q represents Q6, R¹⁰ represents a methyl group, and a combination of R^{X3}, R^{X4}, R^{X5} and R^{X6} represents any combination described in Combination F (hereinafter, referred to as Compound group SX1149).

A compound according to Compound (1S) wherein R¹ represents a methyl group, Q represents Q7, L² represents an oxygen atom, R¹⁰ represents a methyl group, and a combination of R^{X3}, R^{X4}, R^{X5} and R^{X6} represents any combination described in Combination F (hereinafter, referred to as Compound group SX1150).

A compound according to Compound (1S) wherein R¹ represents a chlorine atom, Q represents Q7, L² represents an oxygen atom, R¹⁰ represents a methyl group, and a combination of R^{X3}, R^{X4}, R^{X5} and R^{X6} represents any combination described in Combination F (hereinafter, referred to as Compound group SX1151).

A compound according to Compound (1S) wherein R¹ represents a methyl group, Q represents Q7, L² represents NH, R¹⁰ represents a methyl group, and a combination of R^{X3}, R^{X4}, R^{X5} and R^{X6} represents any combination described in Combination F (hereinafter, referred to as Compound group SX1152).

A compound according to Compound (1S) wherein R¹ represents a chlorine atom, Q represents Q7, L² represents NH, R¹⁰ represents a methyl group, and a combination of R^{X3}, R^{X4}, R^{X5} and R^{X6} represents any combination described in Combination F (hereinafter, referred to as Compound group SX1153).

A compound according to Compound (1S) wherein R¹ represents a methyl group, Q represents Q8A, R¹⁰ represents a methyl group, and a combination of R^{X3}, R^{X4}, R^{X5} and R^{X6} represents any combination described in Combination F (hereinafter, referred to as Compound group SX1154).

A compound according to Compound (1S) wherein R¹ represents a chlorine atom, Q represents Q8A, R¹⁰ represents a methyl group, and a combination of R^{X3}, R^{X4}, R^{X5} and R^{X6} represents any combination described in Combination F (hereinafter, referred to as Compound group SX1155).

A compound according to Compound (1S) wherein R¹ represents a methyl group, Q represents Q8D, R¹⁰ represents a methyl group, and a combination of R^{X3}, R^{X4}, R^{X5} and R^{X6} represents any combination described in Combination F (hereinafter, referred to as Compound group SX1156).

A compound according to Compound (1S) wherein R¹ represents a chlorine atom, Q represents Q8D, R¹⁰ represents a methyl group, and a combination of R^{X3}, R^{X4}, R^{X5} and R^{X6} represents any combination described in Combination F (hereinafter, referred to as Compound group SX1157).

A compound according to Compound (1S) wherein R¹ represents a methyl group, Q represents Q8J, R¹⁰ represents a methyl group, and a combination of R^{X3}, R^{X4}, R^{X5} and R^{X6} represents any combination described in Combination F (hereinafter, referred to as Compound group SX1158).

A compound according to Compound (1S) wherein R¹ represents a chlorine atom, Q represents Q8J, R¹⁰ represents a methyl group, and a combination of R^{X3}, R^{X4}, R^{X5} and R^{X6} represents any combination described in Combination F (hereinafter, referred to as Compound group SX1159).

A compound according to Compound (1S) wherein R¹ represents a methyl group, Q represents Q1, R¹⁰ represents an ethyl group, and a combination of R^{X3}, R^{X4}, R^{X5} and R^{X6} represents any combination described in Combination F (hereinafter, referred to as Compound group SX1160).

A compound according to Compound (1S) wherein R¹ represents a chlorine atom, Q represents Q1, R¹⁰ represents an ethyl group, and a combination of R^{X3}, R^{X4}, R^{X5} and R^{X6} represents any combination described in Combination F (hereinafter, referred to as Compound group SX1161).

A compound according to Compound (1S) wherein R¹ represents an ethyl group, Q represents Q1, R¹⁰ represents an ethyl group, and a combination of R^{X3}, R^{X4}, R^{X5} and R^{X6} represents any combination described in Combination F (hereinafter, referred to as Compound group SX1162).

A compound according to Compound (1S) wherein R¹ represents a fluorine atom, Q represents Q1, R¹⁰ represents an ethyl group, and a combination of R^{X3}, R^{X4}, R^{X5} and R^{X6} represents any combination described in Combination F (hereinafter, referred to as Compound group SX1163).

A compound according to Compound (1S) wherein R¹ represents a cyclopropyl group, Q represents Q1, R¹⁰ represents an ethyl group, and a combination of R^{X3}, R^{X4}, R^{X5} and R^{X6} represents any combination described in Combination F (hereinafter, referred to as Compound group SX1164).

A compound according to Compound (1S) wherein R¹ represents a methyl group, Q represents Q2, L¹ represents an oxygen atom, R¹⁰ represents an ethyl group, and a combination of R^{X3}, R^{X4}, R^{X5} and R^{X6} represents any combination described in Combination F (hereinafter, referred to as Compound group SX1165).

A compound according to Compound (1S) wherein R¹ represents a chlorine atom, Q represents Q2, L¹ represents an oxygen atom, R¹⁰ represents an ethyl group, and a combination of R^{X3}, R^{X4}, R^{X5} and R^{X6} represents any combination described in Combination F (hereinafter, referred to as Compound group SX1166).

A compound according to Compound (1S) wherein R¹ represents a methyl group, Q represents Q2, L¹ represents NH, R¹⁰ represents an ethyl group, and a combination of R^{X3}, R^{X4}, R^{X5} and R^{X6} represents any combination described in Combination F (hereinafter, referred to as Compound group SX1167).

A compound according to Compound (1S) wherein R¹ represents a chlorine atom, Q represents Q2, L¹ represents NH, R¹⁰ represents an ethyl group, and a combination of R^{X3}, R^{X4}, R^{X5} and R^{X6} represents any combination described in Combination F (hereinafter, referred to as Compound group SX1168).

A compound according to Compound (1S) wherein R¹ represents a methyl group, Q represents Q3, R³ represents a methoxy group, R¹⁰ represents an ethyl group, and a combination of R^{X3}, R^{X4}, R^{X5} and R^{X6} represents any combination described in Combination F (hereinafter, referred to as Compound group SX1169).

A compound according to Compound (1S) wherein R¹ represents a chlorine atom, Q represents Q3, R³ represents a methoxy group, R¹⁰ represents an ethyl group, and a combination of R^{X3}, R^{X4}, R^{X5} and R^{X6} represents any combination described in Combination F (hereinafter, referred to as Compound group SX1170).

A compound according to Compound (1S) wherein R¹ represents a methyl group, Q represents Q4, R¹⁰ represents an ethyl group, and a combination of R^{X3}, R^{X4}, R^{X5} and R^{X6} represents any combination described in Combination F (hereinafter, referred to as Compound group SX1171).

A compound according to Compound (1S) wherein R¹ represents a chlorine atom, Q represents Q4, R¹⁰ represents an ethyl group, and a combination of R^{X3}, R^{X4}, R^{X5} and R^{X6} represents any combination described in Combination F (hereinafter, referred to as Compound group SX1172).

A compound according to Compound (1S) wherein R¹ represents a methyl group, Q represents Q5, R¹⁰ represents an ethyl group, and a combination of R^{X3}, R^{X4}, R^{X5} and R^{X6} represents any combination described in Combination F (hereinafter, referred to as Compound group SX1173).

A compound according to Compound (1S) wherein R¹ represents a chlorine atom, Q represents Q5, R¹⁰ represents an ethyl group, and a combination of R^{X3}, R^{X4}, R^{X5} and R^{X6} represents any combination described in Combination F (hereinafter, referred to as Compound group SX1174).

A compound according to Compound (1S) wherein R¹ represents a methyl group, Q represents Q6, R¹⁰ represents an ethyl group, and a combination of R^{X3}, R^{X4}, R^{X5} and R^{X6} represents any combination described in Combination F (hereinafter, referred to as Compound group SX1175).

A compound according to Compound (1S) wherein R¹ represents a chlorine atom, Q represents Q6, R¹⁰ represents an ethyl group, and a combination of R^{X3}, R^{X4}, R^{X5} and R^{X6} represents any combination described in Combination F (hereinafter, referred to as Compound group SX1176).

A compound according to Compound (1S) wherein R¹ represents a methyl group, Q represents Q7, L² represents an oxygen atom, R¹⁰ represents an ethyl group, and a combination of R^{X3}, R^{X4}, R^{X5} and R^{X6} represents any combination described in Combination F (hereinafter, referred to as Compound group SX1177).

A compound according to Compound (1S) wherein R¹ represents a chlorine atom, Q represents Q7, L² represents an oxygen atom, R¹⁰ represents an ethyl group, and a combination of R^{X3}, R^{X4}, R^{X5} and R^{X6} represents any combination described in Combination F (hereinafter, referred to as Compound group SX1178).

A compound according to Compound (1S) wherein R¹ represents a methyl group, Q represents Q7, L² represents NH, R¹⁰ represents an ethyl group, and a combination of R^{X3}, R^{X4}, R^{X5} and R^{X6} represents any combination described in Combination F (hereinafter, referred to as Compound group SX1179).

A compound according to Compound (1S) wherein R¹ represents a chlorine atom, Q represents Q7, L² represents NH, R¹⁰ represents an ethyl group, and a combination of R^{X3}, R^{X4}, R^{X5} and R^{X6} represents any combination described in Combination F (hereinafter, referred to as Compound group SX1180).

A compound according to Compound (1S) wherein R¹ represents a methyl group, Q represents Q8A, R¹⁰ represents an ethyl group, and a combination of R^{X3}, R^{X4}, R^{X5} and R^{X6} represents any combination described in Combination F (hereinafter, referred to as Compound group SX1181).

A compound according to Compound (1S) wherein R¹ represents a chlorine atom, Q represents Q8A, R¹⁰ represents an ethyl group, and a combination of R^{X3}, R^{X4}, R^{X5} and R^{X6} represents any combination described in Combination F (hereinafter, referred to as Compound group SX1182).

A compound according to Compound (1S) wherein R¹ represents a methyl group, Q represents Q8D, R¹⁰ represents an ethyl group, and a combination of R^{X3}, R^{X4}, R^{X5} and R^{X6} represents any combination described in Combination F (hereinafter, referred to as Compound group SX1183).

A compound according to Compound (1S) wherein R¹ represents a chlorine atom, Q represents Q8D, R¹⁰ represents an ethyl group, and a combination of R^{X3}, R^{X4}, R^{X5} and R^{X6} represents any combination described in Combination F (hereinafter, referred to as Compound group SX1184).

A compound according to Compound (1S) wherein R¹ represents a methyl group, Q represents Q8J, R¹⁰ represents an ethyl group, and a combination of R^{X3}, R^{X4}, R^{X5} and R^{X6} represents any combination described in Combination F (hereinafter, referred to as Compound group SX1185).

A compound according to Compound (1S) wherein R¹ represents a chlorine atom, Q represents Q8J, R¹⁰ represents an ethyl group, and a combination of R^{X3}, R^{X4}, R^{X5} and R^{X6} represents any combination described in Combination F (hereinafter, referred to as Compound group SX1186).

A compound according to Compound (1S) wherein R¹ represents a methyl group, Q represents Q1, R¹⁰ represents a cyclopropyl group, and a combination of R^{X3}, R^{X4}, R^{X5} and R^{X6} represents any combination described in Combination F (hereinafter, referred to as Compound group SX1187).

A compound according to Compound (1S) wherein R¹ represents a chlorine atom, Q represents Q1, R¹⁰ represents a cyclopropyl group, and a combination of R^{X3}, R^{X4}, R^{X5} and R^{X6} represents any combination described in Combination F (hereinafter, referred to as Compound group SX1188).

A compound according to Compound (1S) wherein R¹ represents an ethyl group, Q represents Q1, R¹⁰ represents a cyclopropyl group, and a combination of R^{X3}, R^{X4}, R^{X5} and R^{X6} represents any combination described in Combination F (hereinafter, referred to as Compound group SX1189).

A compound according to Compound (1S) wherein R¹ represents a fluorine atom, Q represents Q1, R¹⁰ represents a cyclopropyl group, and a combination of R^{X3}, R^{X4}, R^{X5} and R^{X6} represents any combination described in Combination F (hereinafter, referred to as Compound group SX1190).

A compound according to Compound (1S) wherein R¹ represents a cyclopropyl group, Q represents Q1, R¹⁰ represents a cyclopropyl group, and a combination of R^{X3}, R^{X4}, R^{X5} and R^{X6} represents any combination described in Combination F (hereinafter, referred to as Compound group SX1191).

A compound according to Compound (1S) wherein R¹ represents a methyl group, Q represents Q2, L¹ represents an oxygen atom, R¹⁰ represents a cyclopropyl group, and a combination of R^{X3}, R^{X4}, R^{X5} and R^{X6} represents any combination described in Combination F (hereinafter, referred to as Compound group SX1192).

A compound according to Compound (1S) wherein R¹ represents a chlorine atom, Q represents Q2, L¹ represents an oxygen atom, R¹⁰ represents a cyclopropyl group, and a combination of R^{X3}, R^{X4}, R^{X5} and R^{X6} represents any combination described in Combination F (hereinafter, referred to as Compound group SX1193).

A compound according to Compound (1S) wherein R¹ represents a methyl group, Q represents Q2, L¹ represents NH, R¹⁰ represents a cyclopropyl group, and a combination of R^{X3}, R^{X4}, R^{X5} and R^{X6} represents any combination described in Combination F (hereinafter, referred to as Compound group SX1194).

A compound according to Compound (1S) wherein R¹ represents a chlorine atom, Q represents Q2, L¹ represents NH, R¹⁰ represents a cyclopropyl group, and a combination of R^{X3}, R^{X4}, R^{X5} and R^{X6} represents any combination described in Combination F (hereinafter, referred to as Compound group SX1195).

A compound according to Compound (1S) wherein R¹ represents a methyl group, Q represents Q3, R³ represents a methoxy group, R¹⁰ represents a cyclopropyl group, and a combination of R^{X3}, R^{X4}, R^{X5} and R^{X6} represents any combination described in Combination F (hereinafter, referred to as Compound group SX1196).

A compound according to Compound (1S) wherein R¹ represents a chlorine atom, Q represents Q3, R³ represents a methoxy group, R¹⁰ represents a cyclopropyl group, and a combination of R^{X3}, R^{X4}, R^{X5} and R^{X6} represents any combination described in Combination F (hereinafter, referred to as Compound group SX1197).

A compound according to Compound (1S) wherein R¹ represents a methyl group, Q represents Q4, R¹⁰ represents a cyclopropyl group, and a combination of R^{X3}, R^{X4}, R^{X5} and R^{X6} represents any combination described in Combination F (hereinafter, referred to as Compound group SX1198).

A compound according to Compound (1S) wherein R¹ represents a chlorine atom, Q represents Q4, R¹⁰ represents a cyclopropyl group, and a combination of R^{X3}, R^{X4}, R^{X5} and R^{X6} represents any combination described in Combination F (hereinafter, referred to as Compound group SX1199).

A compound according to Compound (1S) wherein R¹ represents a methyl group, Q represents Q5, R¹⁰ represents a cyclopropyl group, and a combination of R^{X3}, R^{X4}, R^{X5} and R^{X6} represents any combination described in Combination F (hereinafter, referred to as Compound group SX1200).

A compound according to Compound (1S) wherein R¹ represents a chlorine atom, Q represents Q5, R¹⁰ represents a cyclopropyl group, and a combination of R^{X3}, R^{X4}, R^{X5} and R^{X6} represents any combination described in Combination F (hereinafter, referred to as Compound group SX1201).

A compound according to Compound (1S) wherein R¹ represents a methyl group, Q represents Q6, R¹⁰ represents a cyclopropyl group, and a combination of R^{X3}, R^{X4}, R^{X5} and R^{X6} represents any combination described in Combination F (hereinafter, referred to as Compound group SX1202).

A compound according to Compound (1S) wherein R¹ represents a chlorine atom, Q represents Q6, R¹⁰ represents a cyclopropyl group, and a combination of R^{X3}, R^{X4}, R^{X5} and R^{X6} represents any combination described in Combination F (hereinafter, referred to as Compound group SX1203).

A compound according to Compound (1S) wherein R¹ represents a methyl group, Q represents Q7, L² represents an oxygen atom, R¹⁰ represents a cyclopropyl group, and a combination of R^{X3}, R^{X4}, R^{X5} and R^{X6} represents any combination described in Combination F (hereinafter, referred to as Compound group SX1204).

A compound according to Compound (1S) wherein R¹ represents a chlorine atom, Q represents Q7, L² represents an oxygen atom, R¹⁰ represents a cyclopropyl group, and a combination of R^{X3}, R^{X4}, R^{X5} and R^{X6} represents any combination described in Combination F (hereinafter, referred to as Compound group SX1205).

A compound according to Compound (1S) wherein R¹ represents a methyl group, Q represents Q7, L² represents NH, R¹⁰ represents a cyclopropyl group, and a combination of R^{X3}, R^{X4}, R^{X5} and R^{X6} represents any combination described in Combination F (hereinafter, referred to as Compound group SX1206).

A compound according to Compound (1S) wherein R¹ represents a chlorine atom, Q represents Q7, L² represents NH, R¹⁰ represents a cyclopropyl group, and a combination of R^{X3}, R^{X4}, R^{X5} and R^{X6} represents any combination described in Combination F (hereinafter, referred to as Compound group SX1207).

A compound according to Compound (1S) wherein R¹ represents a methyl group, Q represents Q8A, R¹⁰ represents a cyclopropyl group, and a combination of R^{X3}, R^{X4}, R^{X5} and R^{X6} represents any combination described in Combination F (hereinafter, referred to as Compound group SX1208).

A compound according to Compound (1S) wherein R¹ represents a chlorine atom, Q represents Q8A, R¹⁰ represents a cyclopropyl group, and a combination of R^{X3}, R^{X4}, R^{X5} and R^{X6} represents any combination described in Combination F (hereinafter, referred to as Compound group SX1209).

A compound according to Compound (1S) wherein R¹ represents a methyl group, Q represents Q8D, R¹⁰ represents a cyclopropyl group, and a combination of R^{X3}, R^{X4}, R^{X5} and R^{X6} represents any combination described in Combination F (hereinafter, referred to as Compound group SX1210).

A compound according to Compound (1S) wherein R¹ represents a chlorine atom, Q represents Q8D, R¹⁰ represents a cyclopropyl group, and a combination of R^{X3}, R^{X4}, R^{X5} and R^{X6} represents any combination described in Combination F (hereinafter, referred to as Compound group SX1211).

A compound according to Compound (1S) wherein R¹ represents a methyl group, Q represents Q8J, R¹⁰ represents a cyclopropyl group, and a combination of R^{X3}, R^{X4}, R^{X5} and R^{X6} represents any combination described in Combination F (hereinafter, referred to as Compound group SX1212).

A compound according to Compound (1S) wherein R¹ represents a chlorine atom, Q represents Q8J, R¹⁰ represents a cyclopropyl group, and a combination of R^{X3}, R^{X4}, R^{X5} and R^{X6} represents any combination described in Combination F (hereinafter, referred to as Compound group SX1213).

A compound represented by formula (1T) (hereinafter, referred to as Compound (1T)):

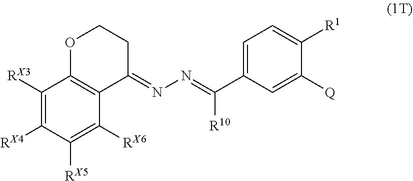

wherein R¹ represents a methyl group, Q represents Q1, R¹⁰ represents a methyl group, and a combination of R^{X3}, R^{X4}, R^{X5} and R^{X6} represents any combination described in Combination F (hereinafter, referred to as Compound group SX1214).

A compound according to Compound (1T) wherein R¹ represents a chlorine atom, Q represents Q1, R¹⁰ represents a methyl group, and a combination of R^{X3}, R^{X4}, R^{X5} and R^{X6} represents any combination described in Combination F (hereinafter, referred to as Compound group SX1215).

A compound according to Compound (1T) wherein R¹ represents an ethyl group, Q represents Q1, R¹⁰ represents a methyl group, and a combination of R^{X3}, R^{X4}, R^{X5} and R^{X6} represents any combination described in Combination F (hereinafter, referred to as Compound group SX1216).

A compound according to Compound (1T) wherein R¹ represents a fluorine atom, Q represents Q1, R¹⁰ represents a methyl group, and a combination of R^{X3}, R^{X4}, R^{X5} and R^{X6} represents any combination described in Combination F (hereinafter, referred to as Compound group SX1217).

A compound according to Compound (1T) wherein R¹ represents a cyclopropyl group, Q represents Q1, R¹⁰ represents a methyl group, and a combination of R^{X3}, R^{X4}, R^{X5} and R^{X6} represents any combination described in Combination F (hereinafter, referred to as Compound group SX1218).

A compound according to Compound (1T) wherein R¹ represents a methyl group, Q represents Q2, L¹ represents an oxygen atom, R¹⁰ represents a methyl group, and a combination of R^{X3}, R^{X4}, R^{X5} and R^{X6} represents any combination described in Combination F (hereinafter, referred to as Compound group SX1219).

A compound according to Compound (1T) wherein R¹ represents a chlorine atom, Q represents Q2, L¹ represents an oxygen atom, R¹⁰ represents a methyl group, and a combination of R^{X3}, R^{X4}, R^{X5} and R^{X6} represents any combination described in Combination F (hereinafter, referred to as Compound group SX1220).

A compound according to Compound (1T) wherein R¹ represents a methyl group, Q represents Q2, L¹ represents NH, R¹⁰ represents a methyl group, and a combination of R^{X3}, R^{X4}, R^{X5} and R^{X6} represents any combination described in Combination F (hereinafter, referred to as Compound group SX1221).

A compound according to Compound (1T) wherein R¹ represents a chlorine atom, Q represents Q2, L¹ represents NH, R¹⁰ represents a methyl group, and a combination of R^{X3}, R^{X4}, R^{X5} and R^{X6} represents any combination described in Combination F (hereinafter, referred to as Compound group SX1222).

A compound according to Compound (1T) wherein R¹ represents a methyl group, Q represents Q3, R³ represents a methoxy group, R¹⁰ represents a methyl group, and a combination of R^{X3}, R^{X4}, R^{X5} and R^{X6} represents any combination described in Combination F (hereinafter, referred to as Compound group SX1223).

A compound according to Compound (1T) wherein R¹ represents a chlorine atom, Q represents Q3, R³ represents a methoxy group, R¹⁰ represents a methyl group, and a combination of R^{X3}, R^{X4}, R^{X5} and R^{X6} represents any combination described in Combination F (hereinafter, referred to as Compound group SX1224).

A compound according to Compound (1T) wherein R¹ represents a methyl group, Q represents Q4, R¹⁰ represents a methyl group, and a combination of R^{X3}, R^{X4}, R^{X5} and R^{X6} represents any combination described in Combination F (hereinafter, referred to as Compound group SX1225).

A compound according to Compound (1T) wherein R¹ represents a chlorine atom, Q represents Q4, R¹⁰ represents a methyl group, and a combination of R^{X3}, R^{X4}, R^{X5} and R^{X6} represents any combination described in Combination F (hereinafter, referred to as Compound group SX1226).

A compound according to Compound (1T) wherein R¹ represents a methyl group, Q represents Q5, R¹⁰ represents a methyl group, and a combination of R^{X3}, R^{X4}, R^{X5} and R^{X6} represents any combination described in Combination F (hereinafter, referred to as Compound group SX1227).

A compound according to Compound (1T) wherein R¹ represents a chlorine atom, Q represents Q5, R¹⁰ represents a methyl group, and a combination of R^{X3}, R^{X4}, R^{X5} and R^{X6} represents any combination described in Combination F (hereinafter, referred to as Compound group SX1228).

A compound according to Compound (1T) wherein R¹ represents a methyl group, Q represents Q6, R¹⁰ represents a methyl group, and a combination of R^{X3}, R^{X4}, R^{X5} and R^{X6} represents any combination described in Combination F (hereinafter, referred to as Compound group SX1229).

A compound according to Compound (1T) wherein R¹ represents a chlorine atom, Q represents Q6, R¹⁰ represents a methyl group, and a combination of R^{X3}, R^{X4}, R^{X5} and R^{X6} represents any combination described in Combination F (hereinafter, referred to as Compound group SX1230).

A compound according to Compound (1T) wherein R¹ represents a methyl group, Q represents Q7, L² represents an oxygen atom, R¹⁰ represents a methyl group, and a combination of R^{X3}, R^{X4}, R^{X5} and R^{X6} represents any combination described in Combination F (hereinafter, referred to as Compound group SX1231).

A compound according to Compound (1T) wherein R¹ represents a chlorine atom, Q represents Q7, L² represents an oxygen atom, R¹⁰ represents a methyl group, and a combination of R^{X3}, R^{X4}, R^{X5} and R^{X6} represents any combination described in Combination F (hereinafter, referred to as Compound group SX1232).

A compound according to Compound (1T) wherein R¹ represents a methyl group, Q represents Q7, L² represents NH, R¹⁰ represents a methyl group, and a combination of R^{X3}, R^{X4}, R^{X5} and R^{X6} represents any combination described in Combination F (hereinafter, referred to as Compound group SX1233).

A compound according to Compound (1T) wherein R¹ represents a chlorine atom, Q represents Q7, L² represents NH, R¹⁰ represents a methyl group, and a combination of R^{X3}, R^{X4}, R^{X5} and R^{X6} represents any combination described in Combination F (hereinafter, referred to as Compound group SX1234).

A compound according to Compound (1T) wherein R¹ represents a methyl group, Q represents Q8A, R¹⁰ represents a methyl group, and a combination of R^{X3}, R^{X4}, R^{X5} and R^{X6} represents any combination described in Combination F (hereinafter, referred to as Compound group SX1235).

A compound according to Compound (1T) wherein R¹ represents a chlorine atom, Q represents Q8A, R¹⁰ represents a methyl group, and a combination of R^{X3}, R^{X4}, R^{X5} and R^{X6} represents any combination described in Combination F (hereinafter, referred to as Compound group SX1236).

A compound according to Compound (1T) wherein R¹ represents a methyl group, Q represents Q8D, R¹⁰ represents a methyl group, and a combination of R^{X3}, R^{X4}, R^{X5} and R^{X6} represents any combination described in Combination F (hereinafter, referred to as Compound group SX1237).

A compound according to Compound (1T) wherein R¹ represents a chlorine atom, Q represents Q8D, R¹⁰ represents a methyl group, and a combination of R^{X3}, R^{X4}, R^{X5} and R^{X6} represents any combination described in Combination F (hereinafter, referred to as Compound group SX1238).

A compound according to Compound (1T) wherein R¹ represents a methyl group, Q represents Q8J, R¹⁰ represents a methyl group, and a combination of R^{X3}, R^{X4}, R^{X5} and R^{X6} represents any combination described in Combination F (hereinafter, referred to as Compound group SX1239).

A compound according to Compound (1T) wherein R¹ represents a chlorine atom, Q represents Q8J, R¹⁰ represents a methyl group, and a combination of R^{X3}, R^{X4}, R^{X5} and R^{X6} represents any combination described in Combination F (hereinafter, referred to as Compound group SX1240).

A compound according to Compound (1T) wherein R¹ represents a methyl group, Q represents Q1, R¹⁰ represents an ethyl group, and a combination of R^{X3}, R^{X4}, R^{X5} and R^{X6} represents any combination described in Combination F (hereinafter, referred to as Compound group SX1241).

A compound according to Compound (1T) wherein R¹ represents a chlorine atom, Q represents Q1, R¹⁰ represents an ethyl group, and a combination of R^{X3}, R^{X4}, R^{X5} and R^{X6} represents any combination described in Combination F (hereinafter, referred to as Compound group SX1242).

A compound according to Compound (1T) wherein R¹ represents an ethyl group, Q represents Q1, R¹⁰ represents an ethyl group, and a combination of R^{X3}, R^{X4}, R^{X5} and R^{X6} represents any combination described in Combination F (hereinafter, referred to as Compound group SX1243).

A compound according to Compound (1T) wherein R¹ represents a fluorine atom, Q represents Q1, R¹⁰ represents an ethyl group, and a combination of R^{X3}, R^{X4}, R^{X5} and R^{X6} represents any combination described in Combination F (hereinafter, referred to as Compound group SX1244).

A compound according to Compound (1T) wherein R¹ represents a cyclopropyl group, Q represents Q1, R¹⁰ represents an ethyl group, and a combination of R^{X3}, R^{X4}, R^{X5} and R^{X6} represents any combination described in Combination F (hereinafter, referred to as Compound group SX1245).

A compound according to Compound (1T) wherein R¹ represents a methyl group, Q represents Q2, L¹ represents an oxygen atom, R¹⁰ represents an ethyl group, and a combination of R^{X3}, R^{X4}, R^{X5} and R^{X6} represents any combination described in Combination F (hereinafter, referred to as Compound group SX1246).

A compound according to Compound (1T) wherein R¹ represents a chlorine atom, Q represents Q2, L¹ represents an oxygen atom, R¹⁰ represents an ethyl group, and a combination of R^{X3}, R^{X4}, R^{X5} and R^{X6} represents any combination described in Combination F (hereinafter, referred to as Compound group SX1247).

A compound according to Compound (1T) wherein R¹ represents a methyl group, Q represents Q2, L¹ represents NH, R¹⁰ represents an ethyl group, and a combination of R^{X3}, R^{X4}, R^{X5} and R^{X6} represents any combination described in Combination F (hereinafter, referred to as Compound group SX1248).

A compound according to Compound (1T) wherein R¹ represents a chlorine atom, Q represents Q2, L¹ represents NH, R¹⁰ represents an ethyl group, and a combination of R^{X3}, R^{X4}, R^{X5} and R^{X6} represents any combination described in Combination F (hereinafter, referred to as Compound group SX1249).

A compound according to Compound (1T) wherein R¹ represents a methyl group, Q represents Q3, R³ represents a methoxy group, R¹⁰ represents an ethyl group, and a combination of R^{X3}, R^{X4}, R^{X5} and R^{X6} represents any combination described in Combination F (hereinafter, referred to as Compound group SX1250).

A compound according to Compound (1T) wherein R¹ represents a chlorine atom, Q represents Q3, R³ represents a methoxy group, R¹⁰ represents an ethyl group, and a combination of R^{X3}, R^{X4}, R^{X5} and R^{X6} represents any combination described in Combination F (hereinafter, referred to as Compound group SX1251).

A compound according to Compound (1T) wherein R¹ represents a methyl group, Q represents Q4, R¹⁰ represents an ethyl group, and a combination of R^{X3}, R^{X4}, R^{X5} and R^{X6} represents any combination described in Combination F (hereinafter, referred to as Compound group SX1252).

A compound according to Compound (1T) wherein R¹ represents a chlorine atom, Q represents Q4, R¹⁰ represents an ethyl group, and a combination of R^{X3}, R^{X4}, R^{X5} and R^{X6} represents any combination described in Combination F (hereinafter, referred to as Compound group SX1253).

A compound according to Compound (1T) wherein R¹ represents a methyl group, Q represents Q5, R¹⁰ represents an ethyl group, and a combination of R^{X3}, R^{X4}, R^{X5} and R^{X6} represents any combination described in Combination F (hereinafter, referred to as Compound group SX1254).

A compound according to Compound (1T) wherein R¹ represents a chlorine atom, Q represents Q5, R¹⁰ represents an ethyl group, and a combination of R^{X3}, R^{X4}, R^{X5} and R^{X6} represents any combination described in Combination F (hereinafter, referred to as Compound group SX1255).

A compound according to Compound (1T) wherein R¹ represents a methyl group, Q represents Q6, R¹⁰ represents an ethyl group, and a combination of R^{X3}, R^{X4}, R^{X5} and R^{X6} represents any combination described in Combination F (hereinafter, referred to as Compound group SX1256).

A compound according to Compound (1T) wherein R¹ represents a chlorine atom, Q represents Q6, R¹⁰ represents an ethyl group, and a combination of R^{X3}, R^{X4}, R^{X5} and R^{X6} represents any combination described in Combination F (hereinafter, referred to as Compound group SX1257).

A compound according to Compound (1T) wherein R¹ represents a methyl group, Q represents Q7, L² represents an oxygen atom, R¹⁰ represents an ethyl group, and a combination of R^{X3}, R^{X4}, R^{X5} and R^{X6} represents any combination described in Combination F (hereinafter, referred to as Compound group SX1258).

A compound according to Compound (1T) wherein R¹ represents a chlorine atom, Q represents Q7, L² represents an oxygen atom, R¹⁰ represents an ethyl group, and a combination of R^{X3}, R^{X4}, R^{X5} and R^{X6} represents any combination described in Combination F (hereinafter, referred to as Compound group SX1259).

A compound according to Compound (1T) wherein R¹ represents a methyl group, Q represents Q7, L² represents NH, R¹⁰ represents an ethyl group, and a combination of R^{X3}, R^{X4}, R^{X5} and R^{X6} represents any combination described in Combination F (hereinafter, referred to as Compound group SX1260).

A compound according to Compound (1T) wherein R¹ represents a chlorine atom, Q represents Q7, L² represents NH, R¹⁰ represents an ethyl group, and a combination of R^{X3}, Rx, R^{X5} and R^{X6} represents any combination described in Combination F (hereinafter, referred to as Compound group SX1261).

A compound according to Compound (1T) wherein R¹ represents a methyl group, Q represents Q8A, R¹⁰ represents an ethyl group, and a combination of R^{X3}, R^{X4}, R^{X5} and R^{X6} represents any combination described in Combination F (hereinafter, referred to as Compound group SX1262).

A compound according to Compound (1T) wherein R¹ represents a chlorine atom, Q represents Q8A, R¹⁰ represents an ethyl group, and a combination of R^{X3}, R^{X4}, R^{X5} and R^{X6} represents any combination described in Combination F (hereinafter, referred to as Compound group SX1263).

A compound according to Compound (1T) wherein R¹ represents a methyl group, Q represents Q8D, R¹⁰ represents an ethyl group, and a combination of R^{X3}, R^{X4}, R^{X5} and R^{X6} represents any combination described in Combination F (hereinafter, referred to as Compound group SX1264).

A compound according to Compound (1T) wherein R¹ represents a chlorine atom, Q represents Q8D, R¹⁰ represents an ethyl group, and a combination of R^{X3}, R^{X4}, R^{X5} and R^{X6} represents any combination described in Combination F (hereinafter, referred to as Compound group SX1265).

A compound according to Compound (1T) wherein R¹ represents a methyl group, Q represents Q8J, R¹⁰ represents an ethyl group, and a combination of R^{X3}, R^{X4}, R^{X5} and R^{X6} represents any combination described in Combination F (hereinafter, referred to as Compound group SX1266).

A compound according to Compound (1T) wherein R¹ represents a chlorine atom, Q represents Q8J, R¹⁰ represents an ethyl group, and a combination of R^{X3}, R^{X4}, R^{X5} and R^{X6} represents any combination described in Combination F (hereinafter, referred to as Compound group SX1267).

A compound according to Compound (1T) wherein R¹ represents a methyl group, Q represents Q1, R¹⁰ represents a cyclopropyl group, and a combination of R^{X3}, R^{X4}, R^{X5} and R^{X6} represents any combination described in Combination F (hereinafter, referred to as Compound group SX1268).

A compound according to Compound (1T) wherein R¹ represents a chlorine atom, Q represents Q1, R¹⁰ represents a cyclopropyl group, and a combination of R^{X3}, R^{X4}, R^{X5} and R^{X6} represents any combination described in Combination F (hereinafter, referred to as Compound group SX1269).

A compound according to Compound (1T) wherein R¹ represents an ethyl group, Q represents Q1, R¹⁰ represents a cyclopropyl group, and a combination of R^{X3}, R^{X4}, R^{X5} and R^{X6} represents any combination described in Combination F (hereinafter, referred to as Compound group SX1270).

A compound according to Compound (1T) wherein R¹ represents a fluorine atom, Q represents Q1, R¹⁰ represents a cyclopropyl group, and a combination of R^{X3}, R^{X4}, R^{X5} and R^{X6} represents any combination described in Combination F (hereinafter, referred to as Compound group SX1271).

A compound according to Compound (1T) wherein R¹ represents a cyclopropyl group, Q represents Q1, R¹⁰ represents a cyclopropyl group, and a combination of R^{X3}, R^{X4}, R^{X5} and R^{X6} represents any combination described in Combination F (hereinafter, referred to as Compound group SX1272).

A compound according to Compound (1T) wherein R¹ represents a methyl group, Q represents Q2, L¹ represents an oxygen atom, R¹⁰ represents a cyclopropyl group, and a combination of R^{X3}, R^{X4}, R^{X5} and R^{X6} represents any combination described in Combination F (hereinafter, referred to as Compound group SX1273).

A compound according to Compound (1T) wherein R¹ represents a chlorine atom, Q represents Q2, L¹ represents an oxygen atom, R¹⁰ represents a cyclopropyl group, and a combination of R^{X3}, R^{X4}, R^{X5} and R^{X6} represents any combination described in Combination F (hereinafter, referred to as Compound group SX1274).

A compound according to Compound (1T) wherein R¹ represents a methyl group, Q represents Q2, L¹ represents NH, R¹⁰ represents a cyclopropyl group, and a combination of R^{X3}, R^{X4}, R^{X5} and R^{X6} represents any combination described in Combination F (hereinafter, referred to as Compound group SX1275).

A compound according to Compound (1T) wherein R¹ represents a chlorine atom, Q represents Q2, L¹ represents NH, R¹⁰ represents a cyclopropyl group, and a combination of R^{X3}, R^{X4}, R^{X5} and R^{X6} represents any combination described in Combination F (hereinafter, referred to as Compound group SX1276).

A compound according to Compound (1T) wherein R¹ represents a methyl group, Q represents Q3, R³ represents a methoxy group, R¹⁰ represents a cyclopropyl group, and a combination of R^{X3}, R^{X4}, R^{X5} and R^{X6} represents any combination described in Combination F (hereinafter, referred to as Compound group SX1277).

A compound according to Compound (1T) wherein R¹ represents a chlorine atom, Q represents Q3, R³ represents a methoxy group, R¹⁰ represents a cyclopropyl group, and a combination of R^{X3}, R^{X4}, R^{X5} and R^{X6} represents any combination described in Combination F (hereinafter, referred to as Compound group SX1278).

A compound according to Compound (1T) wherein R¹ represents a methyl group, Q represents Q4, R¹⁰ represents a cyclopropyl group, and a combination of R^{X3}, R^{X4}, R^{X5} and R^{X6} represents any combination described in Combination F (hereinafter, referred to as Compound group SX1279).

A compound according to Compound (1T) wherein R¹ represents a chlorine atom, Q represents Q4, R¹⁰ represents a cyclopropyl group, and a combination of R^{X3}, R^{X4}, R^{X5} and R^{X6} represents any combination described in Combination F (hereinafter, referred to as Compound group SX1280).

A compound according to Compound (1T) wherein R¹ represents a methyl group, Q represents Q5, R¹⁰ represents a cyclopropyl group, and a combination of R^{X3}, R^{X4}, R^{X5} and R^{X6} represents any combination described in Combination F (hereinafter, referred to as Compound group SX1281).

A compound according to Compound (1T) wherein R¹ represents a chlorine atom, Q represents Q5, R¹⁰ represents a cyclopropyl group, and a combination of R^{X3}, R^{X4}, R^{X5} and R^{X6} represents any combination described in Combination F (hereinafter, referred to as Compound group SX1282).

A compound according to Compound (1T) wherein R¹ represents a methyl group, Q represents Q6, R¹⁰ represents a cyclopropyl group, and a combination of R^{X3}, R^{X4}, R^{X5} and R^{X6} represents any combination described in Combination F (hereinafter, referred to as Compound group SX1283).

A compound according to Compound (1T) wherein R¹ represents a chlorine atom, Q represents Q6, R¹⁰ represents a cyclopropyl group, and a combination of R^{X3}, R^{X4}, R^{X5} and R^{X6} represents any combination described in Combination F (hereinafter, referred to as Compound group SX1284).

A compound according to Compound (1T) wherein R¹ represents a methyl group, Q represents Q7, L² represents an oxygen atom, R¹⁰ represents a cyclopropyl group, and a combination of R^{X3}, R^{X4}, R^{X5} and R^{X6} represents any combination described in Combination F (hereinafter, referred to as Compound group SX1285).

A compound according to Compound (1T) wherein R¹ represents a chlorine atom, Q represents Q7, L² represents an oxygen atom, R¹⁰ represents a cyclopropyl group, and a combination of R^{X3}, R^{X4}, R^{X5} and R^{X6} represents any combination described in Combination F (hereinafter, referred to as Compound group SX1286).

A compound according to Compound (1T) wherein R¹ represents a methyl group, Q represents Q7, L² represents NH, R¹⁰ represents a cyclopropyl group, and a combination of R^{X3}, R^{X4}, R^{X5} and R^{X6} represents any combination described in Combination F (hereinafter, referred to as Compound group SX1287).

A compound according to Compound (1T) wherein R¹ represents a chlorine atom, Q represents Q7, L² represents NH, R¹⁰ represents a cyclopropyl group, and a combination of R^{X3}, R^{X4}, R^{X5} and R^{X6} represents any combination described in Combination F (hereinafter, referred to as Compound group SX1288).

A compound according to Compound (1T) wherein R¹ represents a methyl group, Q represents Q8A, R¹⁰ represents a cyclopropyl group, and a combination of R^{X3}, R^{X4}, R^{X5} and R^{X6} represents any combination described in Combination F (hereinafter, referred to as Compound group SX1289).

A compound according to Compound (1T) wherein R¹ represents a chlorine atom, Q represents Q8A, R¹⁰ represents a cyclopropyl group, and a combination of R^{X3}, R^{X4}, R^{X5} and R^{X6} represents any combination described in Combination F (hereinafter, referred to as Compound group SX1290).

A compound according to Compound (1T) wherein R¹ represents a methyl group, Q represents Q8D, R¹⁰ represents a cyclopropyl group, and a combination of R^{X3}, R^{X4}, R^{X5} and R^{X6} represents any combination described in Combination F (hereinafter, referred to as Compound group SX1291).

A compound according to Compound (1T) wherein R¹ represents a chlorine atom, Q represents Q8D, R¹⁰ represents a cyclopropyl group, and a combination of R^{X3}, R^{X4}, R^{X5} and R^{X6} represents any combination described in Combination F (hereinafter, referred to as Compound group SX1292).

A compound according to Compound (1T) wherein R¹ represents a methyl group, Q represents Q8J, R¹⁰ represents a cyclopropyl group, and a combination of R^{X3}, R^{X4}, R^{X5} and R^{X6} represents any combination described in Combination F (hereinafter, referred to as Compound group SX1293).

A compound according to Compound (1T) wherein R¹ represents a chlorine atom, Q represents Q8J, R¹⁰ represents a cyclopropyl group, and a combination of R^{X3}, R^{X4}, R^{X5} and R^{X6} represents any combination described in Combination F (hereinafter, referred to as Compound group SX1294).

A compound represented by formula (2A) (hereinafter, referred to as Compound (2A)):

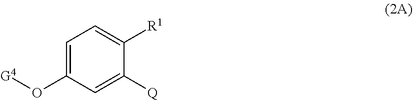

[wherein G⁴ represents any one of formulae G2 to G8, formulae G11 to G21 and formulae G23 to G34.]
wherein R¹ represents a methyl group, Q represents Q1, and a combination of structure of G⁴ and substituents R^{X7}, R^{X8}, R^{X9} and R^{X10} applied to the structure of G⁴ represents any combination described in Combination K (hereinafter, referred to as Compound group SX1295).

Combination K consists of substituent numbers ZK1 to ZK1148. Substituent numbers ZK1 to ZK1148 represent combinations of structure of G⁴ and substituents of R^{X7}, R^{X8}, R^{X9} and R^{X10} applied to the structure of G⁴ according to Compound (2A). And hereinafter, they are referred to as [substituent number; G4, R^{X7},R^{X8},R^{X9},R^{X10}]. For example, the substituent number ZK2 refers to a combination wherein G4 represents G2, R^{X7} represents a methyl group, and each of R^{X8}, R^{X9} and R^{X10} represents a hydrogen atom.
Combination K: [ZK1;G2,H,H,H,H], [ZK2;G2,Me,H,H,H], [ZK3;G2,Et,H,H,H], [ZK4;G 2,Pr,H,H,H], [ZK5;G2,i-Pr,H,H,H], [ZK6;G2,c-Pr,H,H,H], [ZK7;G2,Ph,H,H,H], [ZK8;G2,OMe,H,H,H], [ZK9;G2,OEt, H, H, H], [ZK10; G2, OPh, H, H, H], [ZK11; G2, CF₃, H, H, H], [ZK12; G2, F, H,H,H], [ZK13;G2,Cl,H,H,H], [ZK14;G2,Br,H,H,H], [ZK15;G2,CN,H,H,H], [ZK16;G2,H,Me,H,H], [ZK17;G2,H,Et,H,H], [ZK18;G2,H,Pr,H,H], [ZK19;G2,H,i-Pr,H,H], [ZK20;G2,H,c-Pr,H,H], [ZK21;G2,H,Ph,H,H], [ZK22;G2,H,OMe,H,H], [ZK23;G2,H,O Et,H,H], [ZK24;G2,H,OPh,H,H], [ZK25;G2,H,CF₃,H,H], [ZK26;G2,H, F,H,H], [ZK27;G2,H,Cl,H,H], [ZK28;G2,H,Br,H,H], [ZK29;G2,H,CN, H,H], [ZK30;G2,H,H,Me,H], [ZK31;G2,H,H,Et,H], [ZK32;G2,H,H,Pr, H], [ZK33;G2,H,H,i-Pr,H], [ZK34;G2,H,H,c-Pr,H], [ZK35;G2,H,H,Ph,H], [ZK36;G2,H,H,OMe,H], [ZK37;G2,H,H,O Et,H], [ZK38;G2,H,H,OPh,H], [ZK39;G2,H,H,CF₃,H], [ZK40;G2,H,H, F,H], [ZK41;G2,H,H,Cl,H], [ZK42;G2,H,H,Br,H], [ZK43;G2,H,H,CN, H], [ZK44;G2,H,H,H,Me], [ZK45;G2,H,H,H,Et], [ZK46:G2,H,H,H,Pr], [ZK47;G2,H,H,H,i-Pr], [ZK48;G2,H,H,H,c-Pr], [ZK49;G2,H,H,H,Ph], [ZK50;G2,H,H,H,OMe], [ZK51;G2,H,H,H,O Et], [ZK52;G2,H,H,H,OPh], [ZK53;G2,H,H,H,CF₃], [ZK54;G2,H,H,H, F], [ZK55;G2,H,H,H,Cl], [ZK56;G2,H,H,H,Br], [ZK57;G2,H,H,H,CN], [ZK58;G3,H,H,H,H], [ZK59;G3,Me,H,H,H], [ZK60;G3,Et,H,H,H], [Z K61;G3,Pr,H,H,H], [ZK62;G3,i-Pr,H,H,H], [ZK63;G3,c-Pr,H,H,H], [ZK64;G3,Ph,H,H,H], [ZK65;G3,OMe,H,H,H], [ZK66;G3,O Et,H,H,H], [ZK67;G3,OPh,H,H,H], [ZK68;G3,CF₃,H,H,H], [ZK69;G3, F,H,H,H], [ZK70;G3,Cl,H,H,H], [ZK71;G3,Br,H,H,H], [ZK72;G3,CN, H,H,H], [ZK73;G3,H,Me,H,H], [ZK74;G3,H,Et,H,H], [ZK75;G3,H,Pr, H,H], [ZK76;G3,H,i-Pr,H,H], [ZK77;G3,H,c-Pr,H,H], [ZK78;G3,H,Ph,H,H], [ZK79;G3,H,OMe,H,H], [ZK80;G3,H,O Et,H,H], [ZK81;G3,H,OPh,H,H], [ZK82;G3,H,CF₃,H,H], [ZK83;G3,H, F,H,H], [ZK84;G3,H,Cl,H,H], [ZK85;G3,H,Br,H,H], [ZK86;G3,H,CN, H,H], [ZK87;G3,H,H,Me,H], [ZK88;G3,H,H,Et,H], [ZK89;G3,H,H,Pr, H], [ZK90;G3,H,H,i-Pr,H], [ZK91;G3,H,H,c-Pr,H], [ZK92;G3,H,H,Ph,H], [ZK93;G3,H,H,OMe,H], [ZK94;G3,H,H,O Et,H], [ZK95;G3,H,H,OPh,H], [ZK96;G3,H,H,CF₃,H], [ZK97;G3,H,H, F,H], [ZK98;G3,H,H,Cl,H], [ZK99;G3,H,H,Br,H], [ZK100;G3,H,H,CN,H], [ZK101;G3,H,H,H,Me], [ZK102;G3,H,H,H,Et], [ZK103;G3,H,H,H,Pr], [ZK104;G3,H,H,H,i-Pr], [ZK105;G3,H,H,H,c-Pr], [ZK106;G3,H,H,H,Ph], [ZK107;G3,H,H,H,OMe], [ZK108;G3,H,H, H,OEt], [ZK109;G3,H,H,H,OPh], [ZK110;G3,H,H,H,CF₃], [ZK111;G3, H,H,H,F], [ZK112;G3,H,H,H,Cl], [ZK113;G3,H,H,H,Br], [ZK114;G3, H,H,H,CN], [ZK115;G4,H,H,H,-], [ZK116;G4,Me,H,H,-], [ZK117;G4, Et,H,H,-], [ZK118;G4,Pr,H,H,-], [ZK119;G4,i-Pr,H,H,-], [ZK120;G4,c-Pr,H,H,-], [ZK121;G4,Ph,H,H,-], [ZK122;G4,OMe,H,H,-], [ZK123;G 4,OEt,H,H,-], [ZK124;G4,OPh,H,H,-], [ZK125;G4,CF₃,H,H,-], [ZK1 26;G4,F,H,H,-], [ZK127;G4,Cl,H,H,-], [ZK128;G4,Br,H,H,-], [ZK1 29;G4,CN,H,H,-], [ZK130;G4,H,Me,H,-], [ZK131;G4,H,Et,H,-], [ZK 132;G4,H,Pr,H,-], [ZK133;G4,H,i-Pr,H,-], [ZK134;G4,H,c-Pr,H,-], [ZK135;G4,H,Ph,H,-], [ZK136;G4,H,OMe,H,-], [ZK137;G4, H,OEt,H,-], [ZK138;G4,H,OPh,H,-], [ZK139;G4,H,CF₃,H,-], [ZK140;G4,H,F,H,-], [ZK141;G4,H,Cl,H,-], [ZK142;G4,H,Br,H,-], [ZK143;G4,H,CN,H,-], [ZK144;G4,H,H,Me,-], [ZK145;G4,H,H,Et,-], [ZK14 6;G4,H,H,Pr,-], [ZK147;G4,H,H,i-Pr,-], [ZK148;G4,H,H,c-Pr,-], [ZK149;G4,H,H,Ph,-], [ZK150;G4,H,H,OMe,-], [ZK151;G4,H, H,OEt,-], [ZK152;G4,H,H,OPh,-], [ZK153;G4,H,H,CF₃,-], [ZK154;G 4,H,H,F,-], [ZK155;G4,H,H,Cl,-], [ZK156;G4,H,H,Br,-], [ZK157;G 4,H,H,CN,-], [ZK158;G5,H,H,H,-], [ZK159;G5,Me,H,H,-], [ZK160;G 5,Et,H,H,-], [ZK161;G5,Pr,H,H,-], [ZK162;G5,i-Pr,H,H,-], [ZK163;G5,c-Pr,H,H,-], [ZK164;G5,Ph,H,H,-], [ZK165;G5,OMe,H,H,-], [ZK166;G 5,OEt,H,H,-], [ZK167;G5,OPh,H,H,-], [ZK168;G5,CF₃,H,H,-], [ZK1 69;G5,F,H,H,-], [ZK170;G5,Cl,H,H,-], [ZK171;G5,Br,H,H,-], [ZK1 72;G5,CN,H,H,H-], [ZK173;G5,H,Me,H,-], [ZK174;G5,H,Et,H,-], [ZK 175;G5,H,Pr,H,-], [ZK176;G5,H,i-Pr,H,-], [ZK177;G5,H,c-Pr,H,-], [ZK178;G5,H,Ph,H,-], [ZK179;G5,H,OMe,H,-], [ZK180;G5, H,OEt,H,-], [ZK181;G5,H,OPh,H,-], [ZK182;G5,H,CF₃,H,-], [ZK183;G5,H,F,H,-], [ZK184;G5,H,Cl,H,-], [ZK185;G5,H,Br,H,-], [ZK186;G5,H,CN,H,-], [ZK187;G5,H,H,Me,-], [ZK188;G5,H,H,Et,-], [ZK18 9;G5,H,H,Pr,-], [ZK190;G5,H,H,i-Pr,-], [ZK191;G5,H,H,c-Pr,-], [ZK192;G5,H,H,Ph,-], [ZK193;G5,H,H,OMe,-], [ZK194;G5,H, H,OEt,-], [ZK195;G5,H,H,OPh,-], [ZK196;G5,H,H,CF₃,-], [ZK197;G 5,H,H,F,-], [ZK198;G5,H,H,Cl,-], [ZK199;G5,H,H,Br,-], [ZK200;G 5,H,H,CN,-], [ZK201;G6,H,H,H,-], [ZK202;G6,Me,H,H,-], [ZK203;G 6,Et,H,H,-], [ZK204;G6,Pr,H,H,-], [ZK205;G6,i-Pr,H,H,-], [ZK206;G6,c-Pr,H,H,-], [ZK207;G6,Ph,H,H,-], [ZK208;G6,OMe,H,H,-], [ZK209;G 6,OEt,H,H,-], [ZK210;G6,OPh,H,H,-], [ZK211;G6,CF₃,H,H,-], [ZK2 12;G6,F,H,H,-], [ZK213;G6,Cl,H,H,-], [ZK214;G6,Br,H,H,-], [ZK2 15;G6,CN,H,H,-], [ZK216;G6,H,Me,H,-], [ZK217;G6,H,Et,H,-], [ZK 218;G6,H,Pr,H,-], [ZK219;G6,H,i-Pr,H,-], [ZK220;G6,H,c-Pr,H,-], [ZK221;G6,H,Ph,H,-], [ZK222;G6,H,OMe,H,-], [ZK223;G6, H,OEt,H,-], [ZK224;G6,H,OPh,H,-], [ZK225;G6,H,CF₃,H,-], [ZK226;G6,H,F,H,-], [ZK227;G6,H,Cl,H,-], [ZK228;G6,H,Br,H,-], [ZK229;G6,H,CN,H,-], [ZK230;G6,H,H,Me,-], [ZK231;G6,H,H,Et,-], [ZK23 2;G6,H,H,Pr,-], [ZK233;G6,H,H,i-Pr,-], [ZK234;G6,H,H,c-Pr,-], [ZK235;G6,H,H,Ph,-], [ZK236;G6,H,H,OMe,-], [ZK237;G6,H, H,OEt,-], [ZK238;G6,H,H,OPh,-], [ZK239;G6,H,H,CF₃,-], [ZK240;G 6,H,H,F,-], [ZK241;G6,H,H,Cl,-], [ZK242;G6,H,H,Br,-], [ZK243;G 6,H,H,CN,-], [ZK244;G7,H,H,H,-], [ZK245;G7,Me,H,H,-], [ZK246;G 7,Et,H,H,-], [ZK247;G7,Pr,H,H,-], [ZK248;G7,i-Pr,H,H,-], [ZK249;G7,c-Pr,H,H,-], [ZK250;G7,Ph,H,H,-], [ZK251;G7,OMe,H,H,-], [ZK252;G 7,OEt,H,H,-], [ZK253;G7,OPh,H,H,-], [ZK254;G7,CF₃,H,H,-], [ZK2 55;G7,F,H,H,-], [ZK256;G7,Cl,H,H,-], [ZK257;G7,Br,H,H,-], [ZK2 58;G7,CN,H,H,-], [ZK259;G7,H,Me,H,-], [ZK260;G7,H,Et,H,-], [ZK 261;G7,H,Pr,H,-], [ZK262;G7,H,i-Pr,H,-], [ZK263;G7,H,c-Pr,H,-], [ZK264;G7,H,Ph,H,-], [ZK265;G7,H,OMe,H,-], [ZK266;G7, H,OEt,H,-], [ZK267;G7,H,OPh,H,-], [ZK268;G7,H,CF₃,H,-], [ZK269;G7,H,F,H,-], [ZK270;G7,H,Cl,H,-], [ZK271;G7,H,Br,H,-], [ZK272;G7,H,CN,H,-], [ZK273;G7,H,H,Me,-], [ZK274;G7,H,H,Et,-], [ZK27 5;G7,H,H,Pr,-], [ZK276;G7,H,H,i-Pr,-], [ZK277;G7,H,H,c-Pr,-], [ZK278;G7,H,H,Ph,-], [ZK279;G7,H,H,OMe,-], [ZK280;G7,H, H,OEt,-], [ZK281;G7,H,H,OPh,-], [ZK282;G7,H,H,CF₃,-], [ZK283;G 7,H,H,F,-], [ZK284;G7,H,H,Cl,-], [ZK285;G7,H,H,Br,-], [ZK286;G 7,H,H,CN,-], [ZK287;G8,H,H,H,-], [ZK288;G8,Me,H,H,-], [ZK289;G 8,Et,H,H,-], [ZK290;G8,Pr,H,H,-], [ZK291;G8,i-Pr,H,H,-], [ZK292;G8,c-Pr,H,H,-], [ZK293;G8,Ph,H,H,-], [ZK294;G8,OMe,H,H,-], [ZK295;G 8,OEt,H,H,-], [ZK296;G8,OPh,H,H,-], [ZK297;G8,CF₃,H,H,-], [ZK2 98;G8,F,H,H,-], [ZK299;G8,Cl,H,H,-], [ZK300;G8,Br,H,H,-], [ZK3 01;G8,CN,H,H,-], [ZK302;G8,H,Me,H,-], [ZK303;G8,H,Et,H,-], [ZK 304;G8,H,Pr,H,-], [ZK305;G8,H,i-Pr,H,-], [ZK306;G8,H,c-Pr,H,-], [ZK307;G8,H,Ph,H,-], [ZK308;G8,H,OMe,H,-], [ZK309;G8, H,OEt,H,-], [ZK310;G8,H,OPh,H,-], [ZK311;G8,H,CF₃,H,-], [ZK312;G8,H,F,H,-], [ZK313;G8,H,Cl,H,-], [ZK314;G8,H,Br,H,-], [ZK315;G8,H,CN,H,-], [ZK316;G8,H,H,Me,-], [ZK317;G8,H,H,Et,-], [ZK31 8;G8,H,H,Pr,-], [ZK319;G8,H,H,i-Pr,-], [ZK320;G8,H,H,c-Pr,-], [ZK321;G8,H,H,Ph,-], [ZK322;G8,H,H,OMe,-], [ZK323;G8,H, H,OEt,-], [ZK324;G8,H,H,OPh,-], [ZK325;G8,H,H,CF₃,-], [ZK326;G 8,H,H,F,-], [ZK327;G8,H,H,Cl,-], [ZK328;G8,H,H,Br,-], [ZK329;G 8,H,H,CN,-], [ZK330;G11,H,H,H,-], [ZK331;G11,H,Me,H,-], [ZK332;G11,H,Et,H,-], [ZK333;G11,H,Pr,H,-], [ZK334;G11,H,i-Pr,H,-], [ZK335;G11,H,c-Pr,H,-], [ZK336;G11,H,Ph,H,-], [ZK337;G11,H,OMe,H,-], [ZK338;G 11,H,OEt,H, -], [ZK339;G11,H,OPh,H, -], [ZK340;G11,H,CF₃,H,-], [ZK341;G11,HF,H,-], [ZK342;G11,H,Cl,H,-], [ZK343;G11,H,Br,H,-], [ZK344;G11,H,CN,H,-], [ZK345;G11,H,H,Me,-], [ZK346;G11,H,H,E t,-], [ZK347;G11,H,H,Pr,-], [ZK348;G11,H,H,i-Pr,-], [ZK349;G11,H,H,c-Pr,-], [ZK350;G11,H,H,Ph,-], [ZK351;G11,H,H,OMe,-], [ZK352;G11,H,H,OEt,-], [ZK353;G11,H,H,OPh,-], [ZK354;G11,H,H,CF₃,-], [ZK 355;G11,H,H,F,-], [ZK356;G11,H,H,Cl,-], [ZK357;G11,H,H,Br,-], [ZK358;G11,H,H,CN,-], [ZK359;G11,Me,H,H,-], [ZK360;G11,Me,Me, H,-], [ZK361;G11,Me,Et,H,-], [ZK362;G11,Me,Pr,H,-], [ZK363;G11,Me,i-Pr,H,-], [ZK364;G11,Me,c-Pr,H,-], [ZK365;G11,Me,Ph,H,-], [ZK366;G11,Me,OMe,H,-], [ZK367;G11,Me,OEt,H,-], [ZK368;G11,Me,OPh,H,-], [ZK369;G11,Me,CF₃,H,-], [ZK370;G11,Me,F,H,-], [ZK371;G11,Me,Cl,H,-], [ZK372;G11,M e,Br,H,-], [ZK373;G11,Me,CN,H,-], [ZK374;G11,Me,H,Me,-], [ZK37 5;G11,Me,H,Et,-], [ZK376;G11,Me,H,Pr,-], [ZK377;G11,Me,H,i-Pr,-], [ZK378;G11,Me,H,c-Pr,-], [ZK379;G11,Me,H,Ph,-], [ZK380;G11,Me,H,OMe,-], [ZK381;G 11,Me,H,OEt,-], [ZK382;G11,Me,H,OPh,-], [ZK383;G11,Me,H,CF₃,-], [ZK384;G11,Me,H,F,-], [ZK385;G11,Me,H,Cl,-], [ZK386;G11,Me, H,Br,-], [ZK387;G11,Me,H,CN,-], [ZK388;G12,H,H,H,-], [ZK389;G1 2,Me,H,H,-], [ZK390;G12,Et,H,H,-], [ZK391;G12,Pr,H,H,-], [ZK39 2;G12,i-Pr,H,H,-], [ZK393;G12,c-Pr,H,H,-], [ZK394;G12,Ph,H,H,-], [ZK395;G12,OMe,H,H,-], [ZK396;G12,OEt,H,H,-], [ZK397;G12,OPh,H,H,-], [ZK398;G12,CF₃,H,H,-], [ZK399;G12,F,H,H,-], [ZK400;G12,Cl,H,H,-], [ZK401;G12,Br,H,H,-], [ZK402;G12,CN,H,H,-], [ZK403;G12,H,Me,H,-], [ZK404;G12,H, Et,H,-], [ZK405;G12,H,Pr,H,-], [ZK406;G12,H,i-Pr,H,-], [ZK407;G12,H,c-Pr,H,-], [ZK408;G12,H,Ph,H,-], [ZK409;G12,H,OMe,H,-], [ZK410;G 12,H,OEt,H, -], [ZK411;G12,H,OPh,H, -], [ZK412;G12,H,CF₃,H,-], [ZK413;G12,H,F,H,-], [ZK414;G12,H,Cl,H,-], [ZK415;G12,H,Br,H,-], [ZK416;G12,H,CN,H,-], [ZK417;G12,H,H,Me,-], [ZK418;G12,H,H, Et,-], [ZK419;G12,H,H,Pr,-], [ZK420;G12,H,H,i-Pr,-], [ZK421;G12,H,H,c-Pr,-], [ZK422;G12,H,H,Ph,-], [ZK423;G12,H,H,OMe,-], [ZK424;G12,H,H,OEt,-], [ZK425;G12,H,H,OPh,-], [ZK426;G12,H,H,CF₃,-], [ZK 427;G12,H,H,F,-], [ZK428;G12,H,H,Cl,-], [ZK429;G12,H,H,Br,-], [ZK430;G12,H,H,CN,-], [ZK431;G13,H,H,H,-], [ZK432;G13,Me,H,H,-], [ZK433;G13,Et,H,H,-], [ZK434;G13,Pr,H,H,-], [ZK435;G13,i-Pr,H,H,-], [ZK436;G13,c-Pr,H,H,-], [ZK437;G13,Ph,H,H,-], [ZK438;G13,OMe,H,H,-], [ZK439;G13,OEt,H,H, -], [ZK440;G13,OPh,H,H, -], [ZK441;G13,CF₃,H,H,-], [ZK442;G13,F,H,H,-], [ZK443;G13,Cl,H,H,-], [ZK444;G13,Br,H,H,-], [ZK445;G13,CN,H,H,-], [ZK446;G13,H,H,Me,-], [ZK447;G13,H, H,Et,-], [ZK448;G13,H,H,Pr,-], [ZK449;G13,H,H,i-Pr,-], [ZK450;G13,H,H,c-Pr,-], [ZK451;G13,H,H,Ph,-], [ZK452;G13,H,H,OMe,-], [ZK453;G13,H,H,OEt,-], [ZK454;G13,H,H,OPh,-], [ZK455;G13,H,H,CF₃,-], [ZK 456;G13,H,H,F,-], [ZK457;G13,H,H,Cl,-], [ZK458;G13,H,H,Br,-], [ZK459;G13,H,H,CN,-], [ZK460;G13,H,Me,H,-], [ZK461;G13,Me,Me, H,-], [ZK462;G13,Et,Me,H,-], [ZK463;G13,Pr,Me,H,-], [ZK464;G13,i-Pr,Me,H,-], [ZK465;G13,c-Pr,Me,H,-], [ZK466;G13,Ph,Me,H,-], [ZK467;G13,OMe,Me,H,-], [ZK 468;G13,OEt,Me,H,-], [ZK469;G13,OPh,Me,H,-], [ZK470;G13,CF₃,M e,H,-], [ZK471;G13,F,Me,H,-], [ZK472;G13,Cl,Me,H,-], [ZK473;G1 3,Br,Me,H,-], [ZK474;G13,CN,Me,H,-], [ZK475;G13,H,Me,Me,-], [Z K476;G13,H,Me,Et,-], [ZK477;G13,H,Me,Pr,-], [ZK478;G13,H,Me,i-Pr,-], [ZK479;G13,H,Me,c-Pr,-], [ZK480;G13,H,Me,Ph,-], [ZK481;G13,H,Me,OMe,-], [ZK482;G 13,H,Me,OEt,-], [ZK483;G13,H,Me,OPh,-], [ZK484;G13,H,Me,CF₃,-], [ZK485;G13,H,Me,F,-], [ZK486;G13,H,Me,Cl,-], [ZK487;G13,H,M e,Br,-], [ZK488;G13,H,Me,CN,-], [ZK489;G14,H,H,H,-], [ZK490;G1 4,Me,H,H,-], [ZK491;G14,Et,H,H,-], [ZK492;G14,Pr,H,H,-], [ZK49 3;G14,i-Pr,H,H,-], [ZK494;G14,c-Pr,H,H,-], [ZK495;G14,Ph,H,H,-], [ZK496;G14,OMe,H,H,-], [ZK497;G14,OEt,H,H,-], [ZK498;G14,OPh,H,H,-], [ZK499;G14,CF₃,H,H,-],[ZK500;G14,F,H,H,-], [ZK501;G14,Cl,H,H,-], [ZK502;G14,Br,H,H, -], [ZK503;G14,CN,H,H, -], [ZK504;G14,H,Me,H, -], [ZK505;G14,H, Et,H,-], [ZK506;G14,H,Pr,H,-], [ZK507;G14,H,i-Pr,H,-], [ZK508;G14,H,c-Pr,H,-], [ZK509;G14,H,Ph,H,-], [ZK510;G14,H,OMe,H,-], [ZK511;G 14,H,OEt,H,-], [ZK512;G14,H,OPh,H,-], [ZK513;G14,H,CF₃,H,-], [ZK514;G14,H,F,H,-], [ZK515;G14,H,Cl,H,-], [ZK516;G14,H,Br,H,-], [ZK517;G14,H,CN,H,-], [ZK518;G14,H,H,Me,-], [ZK519;G14,H,H, Et,-], [ZK520;G14,H,H,Pr,-], [ZK521;G14,H,H,i-Pr,-], [ZK522;G14,H,H,c-Pr,-], [ZK523;G14,H,H,Ph,-], [ZK524;G14,H,H,OMe,-], [ZK525;G14,H,H,OEt,-], [ZK526;G14,H,H,OPh,-], [ZK527;G14,H,H,CF₃,-], [ZK 528;G14,H,H,F,-], [ZK529;G14,H,H,Cl,-], [ZK530;G14,H,H,Br,-], [ZK531;G14,H,H,CN,-], [ZK532;G15,H,H,H,-], [ZK533;G15,Me,H,H,-], [ZK534;G15,Et,H,H,-], [ZK535;G15,Pr,H,H,-], [ZK536;G15,i-Pr,H,H,-], [ZK537;G15,c-Pr,H,H,-], [ZK538;G15,Ph,H,H,-], [ZK539;G15,OMe,H,H,-], [ZK540;G15,OEt,H,H,-], [ZK541;G15,OPh,H,H,-], [ZK542;G15,CF₃,H,H,-], [ZK543;G15,F,H,H,-], [ZK544;G15,Cl,H,H,-], [ZK545;G15,Br,H,H,-], [ZK546;G15,CN,H,H,-], [ZK547;G15,H,Me,H,-], [ZK548;G15,H, Et,H,-], [ZK549;G15,H,Pr,H,-], [ZK550;G15,H,i-Pr,H,-], [ZK551;G15,H,c-Pr,H,-], [ZK552;G15,H,Ph,H,-], [ZK553;G15,H,OMe,H,-], [ZK554;G 15,H,OEt,H, -], [ZK555;G15,H,OPh,H, -], [ZK556;G15,H,CF₃,H,-], [ZK557;G15,H,F,H,-], [ZK558;G15,H,Cl,H,-], [ZK559;G15,H,Br,H,-][ZK560;G15,H,CN,H,-], [ZK561;G15,H,H,Me,-], [ZK562;G15,H,H, Et,-], [ZK563;G15,H,H,Pr,-], [ZK564;G15,H,H,i-Pr,-], [ZK565;G15,H,H,C-Pr,-], [ZK566;G15,H,H,Ph,-], [ZK567;G15,H,H,OMe,-], [ZK568;G15,H,H,OEt,-], [ZK569;G15,H,H,OPh,-], [ZK570;G15,H,H,CF₃,-], [ZK 571;G15,H,H,F,-], [ZK572;G15,H,H,Cl,-], [ZK573;G15,H,H,Br,-], [ZK574;G15,H,H,CN,-], [ZK575;G16,H,H,-,-], [ZK576;G16,Me,H,-,-], [ZK577;G16,Et,H,-,-], [ZK578;G16,Pr,H,-,-], [ZK579;G16,i-Pr,H,-,-], [ZK580;G16,c-Pr,H,-,-], [ZK581;G16,Ph,H,-,-], [ZK582;G16,OMe,H,-,-], [ZK583;G16,OEt,H, -, -], [ZK584;G16,OPh,H, -, -], [ZK585;G16,CF₃,H, -,-], [ZK586;G16,F,H,-,-], [ZK587;G16,Cl,H,-,-], [ZK588;G16,Br,H,-,-], [ZK589;G16,CN,H,-,-], [ZK590;G16,H,Me,-,-], [ZK591;G16,H, Et,-,-], [ZK592;G16,H,Pr,-,-], [ZK593;G16,H,i-Pr,-,-], [ZK594;G16,H,c-Pr,-,-], [ZK595;G16,H,Ph,-,-], [ZK596;G16,H,OMe,-,-], [ZK597;G 16,H,OEt,-,-], [ZK598;G16,H,OPh,-,-], [ZK599;G16,H,CF₃,-,-], [ZK600;G16,H,F,-,-], [ZK601;G16,H,Cl,-,-], [ZK602;G16,H,Br,-,-], [ZK603;G16,H,CN,-,-], [ZK604;G17,H,H,-,-], [ZK605;G17,Me,H, -,-], [ZK606;G17,Et,H,-,-], [ZK607;G17,Pr,H,-,-], [ZK608;G17,i-Pr,H,-,-], [ZK609;G17,c-Pr,H,-,-], [ZK610;G17,Ph,H,-,-], [ZK611;G17,OMe,H,-,-], [ZK612;G17,OEt,H, -, -], [ZK613;G17,OPh,H, -, -], [ZK614;G17,CF₃,H, -,-], [ZK615;G17,F,H,-,-], [ZK616;G17,Cl,H,-,-], [ZK617;G17,Br,H,-,-], [ZK618;G17,CN,H,-,-], [ZK619;G17,H,Me,-,-], [ZK620;G17,H, Et,-,-], [ZK621;G17,H,Pr,-,-], [ZK622;G17,H,i-Pr,-,-], [ZK623;G17,H,c-Pr,-,-], [ZK624;G17,H,Ph,-,-], [ZK625;G17,H,OMe,-,-], [ZK626;G 17,H,OEt,-,-], [ZK627;G17,H,OPh,-,-], [ZK628;G17,H,CF₃,-,-], [ZK629;G17,H,F,-,-], [ZK630;G17,H,Cl,-,-], [ZK631;G17,H,Br,-,-], [ZK632;G17,H,CN,-,-], [ZK633;G18,H,H,-,-], [ZK634;G18,Me,H, -,-], [ZK635;G18,Et,H,-,-], [ZK636;G18,Pr,H,-,-], [ZK637;G18,i-Pr,H,-,-], [ZK638;G18,c-Pr,H,-,-], [ZK639;G18,Ph,H,-,-], [ZK640;G18,OMe,H,-,-], [ZK641;G18,OEt,H, -, -], [ZK642;G18,OPh,H, -, -], [ZK643;G18,CF₃,H, -,-], [ZK644;G18,F,H,-,-], [ZK645;G18,Cl,H,-,-], [ZK646;G18,Br,H,-,-], [ZK647;G18,CN,H,-,-], [ZK648;G18,H,Me,-,-], [ZK649;G18,H, Et,-,-], [ZK650;G18,H,Pr,-,-], [ZK651;G18,H,i-Pr,-,-], [ZK652;G18,H,c-Pr,-,-], [ZK653;G18,H,Ph,-,-], [ZK654;G18,H,OMe,-,-], [ZK655;G 18,H,OEt,-,-], [ZK656;G18,H,OPh,-,-], [ZK657;G18,H,CF₃,-,-], [ZK658;G18,H,F,-,-], [ZK659;G18,H,Cl,-,-], [ZK660;G18,H,Br,-,-], [ZK661;G18,H,CN,-,-], [ZK662;G19,H,H,-,-], [ZK663;G19,Me,H, -,-], [ZK664;G19,Et,H,-,-], [ZK665;G19,Pr,H,-,-], [ZK666;G19,i-Pr,H,-,-], [ZK667;G19,c-Pr,H,-,-], [ZK668;G19,Ph,H,-,-], [ZK669;G19,OMe,H,-,-], [ZK670;G19,OEt,H, -, -], [ZK671;G19,OPh,H, -, -], [ZK672;G19,CF₃,H, -,-], [ZK673;G19,F,H,-,-], [ZK674;G19,Cl,H,-,-], [ZK675;G19,Br,H,-,-], [ZK676;G19,CN,H,-,-], [ZK677;G19,H,Me,-,-], [ZK678;G19,H, Et,-,-], [ZK679;G19,H,Pr,-,-], [ZK680;G19,H,i-Pr,-,-], [ZK681;G19,H,c-Pr,-,-], [ZK682;G19,H,Ph,-,-], [ZK683;G19,H,OMe,-,-], [ZK684;G 19,H,OEt,-,-], [ZK685;G19,H,OPh,-,-], [ZK686;G19,H,CF₃,-,-], [ZK687;G19,H,F,-,-], [ZK688;G19,H,Cl,-,-], [ZK689;G19,H,Br,-,-], [ZK690;G19,H,CN,-,-], [ZK691;G20,H,H,-,-], [ZK692;G20,Me,H, -,-], [ZK693;G20,Et,H,-,-], [ZK694;G20,Pr,H,-,-], [ZK695;G20,i-Pr,H,-,-], [ZK696;G20,c-Pr,H,-,-], [ZK697;G20,Ph,H,-,-], [ZK698;G20,OMe,H,-,-], [ZK699;G20,OEt,H, -,-], [ZK700;G20,OPh,H, -,-], [ZK701;G20,CF₃,H,-,-], [ZK702;G20,F,H,-,-], [ZK703;G20,Cl,H,-,-], [ZK704;G20,Br,H,-,-], [ZK705;G20,CN,H,-,-], [ZK706;G20,H,Me,-,-], [ZK707;G20,H, Et,-,-], [ZK708;G20,H,Pr,-,-], [ZK709;G20,H,i-Pr,-,-], [ZK710;G20,H,c-Pr,-,-], [ZK711;G20,H,Ph,-,-], [ZK712;G20,H,OMe,-,-], [ZK713;G 20,H,OEt,-,-], [ZK714;G20,H,OPh,-,-], [ZK715;G20,H,CF₃,-,-], [ZK716;G20,H,F,-,-], [ZK717;G20,H,Cl,-,-], [ZK718;G20,H,Br,-,-], [ZK719;G20,H,CN,-,-], [ZK720;G21,H,H,-,-], [ZK721;G21,Me,H, -,-], [ZK722;G21,Et,H,-,-], [ZK723;G21,Pr,H,-,-], [ZK724;G21,i-Pr,H,-,-], [ZK725;G21,c-Pr,H,-,-], [ZK726;G21,Ph,H,-,-], [ZK727;G21,OMe,H,-,-], [ZK728;G21,OEt,H, -,-], [ZK729;G21,OPh,H, -,-], [ZK730;G21,CF₃,H,-,-], [ZK731;G21,F,H,-,-], [ZK732;G21,Cl,H,-,-], [ZK733;G21,Br,H,-,-], [ZK734;G21,CN,H,-,-], [ZK735;G21,H,Me,-,-], [ZK736;G21,H, Et,-,-], [ZK737;G21,H,Pr,-,-], [ZK738;G21,H,i-Pr,-,-], [ZK739;G21,H,c-Pr,-,-], [ZK740;G21,H,Ph,-,-], [ZK741;G21,H,OMe,-,-], [ZK742;G 21,H,OEt,-,-], [ZK743;G21,H,OPh,-,-], [ZK744;G21,H,CF₃,-,-], [ZK745;G21,H,F,-,-], [ZK746;G21,H,Cl,-,-], [ZK747;G21,H,Br,-,-], [ZK748;G21,H,CN,-,-], [ZK749;G23,H,H,-,-], [ZK750;G23,Me,H, -,-], [ZK751;G23,Et,H,-,-], [ZK752;G23,Pr,H,-,-], [ZK753;G23,i-Pr,H,-,-], [ZK754;G23,c-Pr,H,-,-], [ZK755;G23,Ph,H,-,-], [ZK756;G23,OMe,H,-,-], [ZK757;G23,OEt,H, -,-], [ZK758;G23,OPh,H, -,-], [ZK759;G23,CF₃,H,-,-], [ZK760;G23,F,H,-,-], [ZK761;G23,Cl,H,-,-], [ZK762;G23,Br,H,-,-], [ZK763;G23,CN,H,-,-], [ZK764;G23,H,Me,-,-], [ZK765;G23,H, Et,-,-], [ZK766;G23,H,Pr,-,-], [ZK767;G23,H,i-Pr,-,-], [ZK768;G23,H,c-Pr,-,-], [ZK769;G23,H,Ph,-,-], [ZK770;G23,H,OMe,-,-], [ZK771;G 23,H,OEt,-,-], [ZK772;G23,H,OPh,-,-], [ZK773;G23,H,CF₃,-,-], [ZK774;G23,H,F,-,-], [ZK775;G23,H,Cl,-,-], [ZK776;G23,H,Br,-,-], [ZK777;G23,H,CN,-,-], [ZK778;G24,H,H,-,-], [ZK779;G24,Me,H, -,-], [ZK780;G24,Et,H,-,-], [ZK781;G24,Pr,H,-,-], [ZK782;G24,i-Pr,H,-,-], [ZK783;G24,c-Pr,H,-,-], [ZK784;G24,Ph,H,-,-], [ZK785;G24,OMe,H,-,-], [ZK786;G24,OEt,H, -, -], [ZK787;G24,OPh,H, -, -], [ZK788;G24,CF₃,H, -,-], [ZK789;G24,F,H,-,-], [ZK790;G24,Cl,H,-,-], [ZK791;G24,Br,H,-,-], [ZK792;G24,CCN,H,-,-], [ZK793;G24,H,Me,-,-], [ZK794;G24,H, Et,-,-], [ZK795;G24,H,Pr,-,-], [ZK796;G24,H,i-Pr,-,-], [ZK797;G24,H,c-Pr,-,-], [ZK798;G24,H,Ph,-,-], [ZK799;G24,H,OMe,-,-], [ZK800;G 24,H,OEt,-,-], [ZK801;G24,H,OPh,-,-], [ZK802;G24,H,CF₃,-,-], [ZK803;G24,H,F,-,-], [ZK804;G24,H,Cl,-,-], [ZK805;G24,H,Br,-,-], [ZK806;G24,H,CN,-,-], [ZK807;G25,H,H,H,-], [ZK808;G25,H,Me, H,-], [ZK809;G25,H,Et,H,-], [ZK810;G25,H,Pr,H,-], [ZK811;G25,H,i-Pr,H,-], [ZK812;G25,H,c-Pr,H,-], [ZK813;G25,H,Ph,H,-], [ZK814;G25,H,OMe,H,-], [ZK815;G 25,H,OEt,H, -], [ZK816;G25,H,OPh,H, -], [ZK817;G25,H,CF₃,H,-], [ZK818;G25,H,F,H,-], [ZK819;G25,H,Cl,H,-], [ZK820;G25,H,Br,H,-], [ZK821;G25,H,CN,H,-], [ZK822;G25,H,H,Me,-], [ZK823;G25,H,H, Et,-], [ZK824;G25,H,H,Pr,-], [ZK825;G25,H,H,i-Pr,-], [ZK826;G25,H,H,c-Pr,-], [ZK827;G25,H,H,Ph,-], [ZK828;G25,H,H,OMe,-], [ZK829;G25,H,H,OEt,-], [ZK830;G25,H,H,OPh,-], [ZK831;G25,H,H,CF₃,-], [ZK 832;G25,H,H,F,-], [ZK833;G25,H,H,Cl,-], [ZK834;G25,H,H,Br,-], [ZK835;G25,H,H,CN,-], [ZK836;G25,Me,H,H,-], [ZK837;G25,Me,Me, H,-], [ZK838;G25,Me,Et,H,-], [ZK839;G25,Me,Pr,H,-], [ZK840;G25,Me,i-Pr,H,-], [ZK841;G25,Me,c-Pr,H,-], [ZK842;G25,Me,Ph,H,-], [ZK843;G25,Me,OMe,H,-], [ZK844;G25,Me,OEt,H,-], [ZK845;G25,Me,OPh,H,-], [ZK846;G25,Me,CF₃,H,-], [ZK847;G25,Me,F,H,-], [ZK848;G25,Me,Cl,H,-], [ZK849;G25,M e,Br,H,-], [ZK850;G25,Me,CN,H,-], [ZK851;G25,Me,H,Me,-], [ZK85 2;G25,Me,H,Et,-], [ZK853;G25,Me,H,Pr,-], [ZK854;G25,Me,H,i-Pr,-], [ZK855;G25,Me,H,c-Pr,-], [ZK856;G25,Me,H,Ph,-], [ZK857;G25,Me,H,OMe,-], [ZK858;G 25,Me,H,OEt,-], [ZK859;G25,Me,H,OPh,-], [ZK860;G25,Me,H,CF₃,-], [ZK861;G25,Me,H,F,-], [ZK862;G25,Me,H,Cl,-], [ZK863;G25,Me, H,Br,-], [ZK864;G25,Me,H,CN,-], [ZK865;G26,H,H,H,-], [ZK866;G2 6,H,Me,H,-], [ZK867;G26,H,Et,H,-], [ZK868;G26,H,Pr,H,-], [ZK86 9;G26,H,i-Pr,H,-], [ZK870;G26,H,c-Pr,H,-], [ZK871;G26,H,Ph,H,-], [ZK872;G26,H,OMe,H,-], [ZK873;G 26,H,OEt,H,-], [ZK874;G26,H,OPh,H,-], [ZK875;G26,H,CF₃,H,-], [ZK876;G26,H,F,H,-], [ZK877;G26,H,Cl,H,-], [ZK878;G26,H,Br,H,-], [ZK879;G26,H,CN,H,-], [ZK880;G26,H,H,Me,-], [ZK881;G26,H,H, Et,-], [ZK882;G26,H,H,Pr,-], [ZK883;G26,H,H,i-Pr,-], [ZK884;G26,H,H,c-Pr,-], [ZK885;G26,H,H,Ph,-], [ZK886;G26,H,H,OMe,-], [ZK887;G26,H,H,OEt,-], [ZK888;G26,H,H,OPh,-], [ZK889;G26,H,H,CF₃,-], [ZK 890;G26,H,H,F,-], [ZK891;G26,H,H,Cl,-], [ZK892;G26,H,H,Br,-], [ZK893;G26,H,H,CN,-], [ZK894;G26,Me,H,H,-], [ZK895;G26,Me,Me, H,-], [ZK896;G26,Me,Et,H,-], [ZK897;G26,Me,Pr,H,-], [ZK898;G26,Me,i-Pr,H,-], [ZK899;G26,Me,c-Pr,H,-], [ZK900;G26,Me,Ph,H,-], [ZK901;G26,Me,OMe,H,-], [ZK902;G26,Me,OEt,H,-], [ZK903;G26,Me,OPh,H,-], [ZK904;G26,Me,CF₃,H,-], [ZK905;G26,Me,F,H,-], [ZK906;G26,Me,Cl,H,-], [ZK907;G26,M e,Br,H,-], [ZK908;G26,Me,CN,H,-], [ZK909;G26,Me,H,Me,-], [ZK91 0;G26,Me,H,Et,-], [ZK911;G26,Me,H,Pr,-], [ZK912;G26,Me,H,i-Pr,-], [ZK913;G26,Me,H,c-Pr,-], [ZK914;G26,Me,H,Ph,-], [ZK915;G26,Me,H,OMe,-], [ZK916;G 26,Me,H,OEt,-], [ZK917;G26,Me,H,OPh,-], [ZK918;G26,Me,H,CF₃,-], [ZK919;G26,Me,H,F,-], [ZK920;G26,Me,H,Cl,-], [ZK921;G26,Me, H,Br,-], [ZK922;G26,Me,H,CN,-], [ZK923;G27,H,H,H,-], [ZK924;G2 7,Me,H,H,-], [ZK925;G27,Et,H,H,-], [ZK926;G27,Pr,H,H,-], [ZK92 7;G27,i-Pr,H,H,-], [ZK928;G27,c-Pr,H,H,-], [ZK929;G27,Ph,H,H,-], [ZK930;G27,OMe,H,H,-], [ZK931;G27,OEt,H,H, -], [ZK932;G27,OPh,H,H, -], [ZK933;G27,CF₃,H,H,-], [ZK934;G27,F,H,H,-], [ZK935;G27,Cl,H,H,-], [ZK936;G27,Br,H,H,-], [ZK937;G27,CN,H,H,-], [ZK938;G27,H,Me,H,-], [ZK939;G27,H, Et,H,-], [ZK940;G27,H,Pr,H,-], [ZK941;G27,H,i-Pr,H,-], [ZK942;G27,H,c-Pr,H,-], [ZK943;G27,H,Ph,H,-], [ZK944;G27,H,OMe,H,-], [ZK945;G 27,H,OEt,H, -], [ZK946;G27,H,OPh,H, -], [ZK947;G27,H,CF₃,H,-], [ZK948;G27,H,F,H,-], [ZK949;G27,H,Cl,H,-], [ZK950;G27,H,Br,H,-], [ZK951;G27,H,CN,H,-], [ZK952;G27,H,H,Me,-], [ZK953;G27,H,H, Et,-], [ZK954;G27,H,H,Pr,-], [ZK955;G27,H,H,i-Pr,-], [ZK956;G27,H,H,c-Pr,-], [ZK957;G27,H,H,Ph,-], [ZK958;G27,H,H,OMe,-], [ZK959;G27,H,H,OEt,-], [ZK960;G27,H,H,OPh,-], [ZK961;G27,H,H,CF₃,-], [ZK 962;G27,H,H,F,-], [ZK963;G27,H,H,Cl,-], [ZK964;G27,H,H,Br,-], [ZK965;G27,H,H,CN,-], [ZK966;G28,H,H,-,-], [ZK967;G28,Me,H,-,-], [ZK968;G28,Cl,H,-,-], [ZK969;G28,CF₃,H,-,-], [ZK970;G28,H, Me,-,-], [ZK971;G28,H,Cl,-,-], [ZK972;G28,H,CF₃,-,-], [ZK973;G 28,H,SH,-,-], [ZK974;G29,H,H,H,-], [ZK975;G29,H,Me,H,-], [ZK97 6;G29,H,Et,H,-], [ZK977;G29,H,Pr,H,-], [ZK978;G29,H,i-Pr,H,-], [ZK979;G29,H,c-Pr,H,-], [ZK980;G29,H,Ph,H,-], [ZK981;G29,H,OMe,H,-], [ZK982;G 29,H,OEt,H,-], [ZK983;G29,H,OPh,H,-], [ZK984;G29,H,CF₃,H,-], [ZK985;G29,H,F,H,-], [ZK986;G29,H,Cl,H,-], [ZK987;G29,H,Br,H,-], [ZK988;G29,H,CN,H,-], [ZK989;G29,H,H,Me,-], [ZK990;G29,H,H, Et,-], [ZK991;G29,H,H,Pr,-], [ZK992;G29,H,H,i-Pr,-], [ZK993;G29,H,H,c-Pr,-], [ZK994;G29,H,H,Ph,-], [ZK995;G29,H,H,OMe,-], [ZK996;G29,H,H,OEt,-], [ZK997;G29,H,H,OPh,-], [ZK998;G29,H,H,CF₃,-], [ZK 999;G29,H,H,F,-], [ZK1000;G29,H,H,Cl,-], [ZK1001;G29,H,H,Br,-], [ZK1002;G29,H,H,CN,-], [ZK1003;G29,Me,H,H,-], [ZK1004;G29,M e,Me,H,-], [ZK1005;G29,Me,Et,H,-], [ZK1006;G29,Me,Pr,H,-], [ZK 1007;G29,Me,i-Pr,H,-], [ZK1008;G29,Me,c-Pr,H,-], [ZK1009;G29,Me,Ph,H,-], [ZK1010;G29,Me,OMe,H,-], [ZK1 011;G29,Me,OEt,H,-], [ZK1012;G29,Me,OPh,H,-], [ZK1013;G29,Me, CF₃,H,-], [ZK1014;G29,Me,F,H,-], [ZK1015;G29,Me,Cl,H,-], [ZK10 16;G29,Me,Br,H,-], [ZK1017;G29,Me,CN,H,-], [ZK1018;G29,Me,H,M e,-], [ZK1019;G29,Me,H,Et,-], [ZK1020;G29,Me,H,Pr,-], [ZK1021; G29,Me,H,i-Pr,-], [ZK1022;G29,Me,H,c-Pr,-], [ZK1023;G29,Me,H,Ph,-], [ZK1024;G29,Me,H,OMe,-], [ZK102 5;G29,Me,H,OEt,-], [ZK1026;G29,Me,H,OPh,-], [ZK1027;G29,Me,H, CF₃,-], [ZK1028;G29,Me,H,F,-], [ZK1029;G29,Me,H,Cl,-], [ZK1030;G29,Me,H,Br,-], [ZK1031;G29,Me,H,CN,-], [ZK1032;G30,H,H,H,-],[ZK1033;G30,Me,H,H,-], [ZK1034;G30,Et,H,H,-], [ZK1035;G30,Pr,H,H,-], [ZK1036;G30,i-Pr,H,H,-], [ZK1037;G30,c-Pr,H,H,-], [ZK1038;G30,Ph,H,H,-], [ZK1039;G30,OMe,H,H,-], [ZK1 040;G30,OEt,H,H,-], [ZK1041;G30,OPh,H,H,-], [ZK1042;G30,CF₃,H,H,-], [ZK1043;G30,F,H,H,-], [ZK1044;G30,Cl,H,H,-], [ZK1045;G3 0,Br,H,H,-], [ZK1046;G30,CN,H,H,-], [ZK1047;G30,H,Me,H,-], [ZK 1048;G30,H,Et,H,-], [ZK1049;G30,H,Pr,H,-], [ZK1050;G30,H,i-Pr,H,-], [ZK1051;G30,H,c-Pr,H,-], [ZK1052;G30,H,Ph,H,-], [ZK1053;G30,H,OMe,H,-], [ZK105 4;G30,H,OEt,H,-], [ZK1055;G30,H,OPh,H,-], [ZK1056;G30,H,CF₃,H,-], [ZK1057;G30,H,F,H,-], [ZK1058;G30,H,Cl,H,-], [ZK1059;G30, H,Br,H,-], [ZK1060;G30,H,CN,H,-], [ZK1061;G30,H,H,Me,-], [ZK10 62;G30,H,H,Et,-], [ZK1063;G30,H,H,Pr,-], [ZK1064;G30,H,H,i-Pr,-], [ZK1065;G30,H,H,c-Pr,-], [ZK1066;G30,H,H,Ph,-], [ZK1067;G30,H,H,OMe,-], [ZK1068; G30,H,H,OEt,-], [ZK1069;G30,H,H,OPh,-], [ZK1070;G30,H,H,CF₃,-], [ZK1071;G30,H,H,F,-], [ZK1072;G30,H,H,Cl,-], [ZK1073;G30,H, H,Br,-], [ZK1074;G30,H,H,CN,-], [ZK1075;G30,H,Oi-Pr,H,-], [ZK1076;G30,H,Oc-Pr,H,-], [ZK1077;G30,H,Ot-Bu,H,-], [ZK1078;G30,H,OCF₃,H,-], [ZK1079;G30,H,OCHF₂,H, -], [Z K1080;G30,H,OPr,H,-], [ZK1081;G30,H,OCH2Ph,H,-], [ZK1082;G30, H,OCH2-i-Pr,H,-], [ZK1083;G30,H,OCH2-c-Pr,H,-], [ZK1084;G30,H,OCH2-t-Bu,H,-], [ZK1085;G31,H,-,-,-], [ZK1086;G31,Me,-,-,-], [ZK1087; G31,Et,-,-,-], [ZK1088;G31,Pr,-,-,-], [ZK1089;G31,i-Pr,-,-,-], [ZK1090;G31,c-Pr,-,-,-], [ZK1091;G31,Ph,-,-,-], [ZK1092;G31,OMe,-,-,-], [ZK1 093;G31,OEt,-,-,-], [ZK1094;G31,OPh,-,-,-], [ZK1095;G31,CF₃,-,-,-], [ZK1096;G31,F,-,-,-], [ZK1097;G31,Cl,-,-,-], [ZK1098;G3 1,Br,-,-,-], [ZK1099;G31,CN,-,-,-], [ZK1100;G31,t-Bu,-,-,-], [ZK1101;G32,H,-,-,-], [ZK1102;G32,Me,-,-,-], [ZK110 3;G32,Et,-,-,-], [ZK1104;G32,Pr,-,-,-], [ZK1105;G32,i-Pr,-,-,-], [ZK1106;G32,c-Pr,-,-,-], [ZK1107;G32,Ph,-,-,-], [ZK1108;G32,OMe,-,-,-], [ZK1 109;G32,OEt,-,-,-], [ZK1110;G32,OPh,-,-,-], [ZK1111;G32,CF₃,-,-,-], [ZK1112;G32,F,-,-,-], [ZK1113;G32,Cl,-,-,-], [ZK1114;G3 2,Br,-,-,-], [ZK1115;G32,CN,-,-,-], [ZK1116;G32,t-Bu,-,-,-], [ZK1117;G33,H,-,-,-], [ZK1118;G33,Me,-,-,-], [ZK111 9;G33,Et,-,-,-], [ZK1120;G33,Pr,-,-,-], [ZK1121;G33,i-Pr,-,-,-], [ZK1122;G33,c-Pr,-,-,-], [ZK1123;G33,Ph,-,-,-], [ZK1124;G33,OMe,-,-,-], [ZK1 125;G33,OEt,-,-,-], [ZK1126;G33,OPh,-,-,-], [ZK1127;G33,CF₃,-,-,-], [ZK1128;G33,F,-,-,-], [ZK1129;G33,Cl,-,-,-], [ZK1130;G3 3,Br,-,-,-], [ZK1131;G33,CN,-,-,-], [ZK1132;G33,t-Bu,-,-,-], [ZK1133;G34,H,-,-,-], [ZK1134;G34,Me,-,-,-], [ZK113 5;G34,Et,-,-,-], [ZK1136;G34,Pr,-,-,-], [ZK1137;G34,i-Pr,-,-,-], [ZK1138;G34,c-Pr,-,-,-], [ZK1139;G34,Ph,-,-,-], [ZK1140;G34,OMe,-,-,-], [ZK1 141;G34,OEt,-,-,-], [ZK1142;G34,OPh,-,-,-], [ZK1143;G34,CF₃,-,-,-], [ZK1144;G34,F,-,-,-], [ZK1145;G34,Cl,-,-,-], [ZK1146;G3 4,Br,-,-,-], [ZK1147;G34, CN,-,-,-], [ZK1148;G34, t-Bu, -,-, -]

A compound according to Compound (2A) wherein R¹ represents a chlorine atom, Q represents Q1, and a combination of structure of G⁴ and substituents R^{X7}, R^{X8}, R^{X9} and R^{X10} applied to the structure of G⁴ represents any combination described in Combination K (hereinafter, referred to as Compound group SX1296).

A compound according to Compound (2A) wherein R¹ represents a methyl group, Q represents Q2, L¹ represents an oxygen atom, and a combination of structure of G⁴ and substituents R^{X7}, R^{X8}, R^{X9} and R^{X10} applied to the structure of G⁴ represents any combination described in Combination K (hereinafter, referred to as Compound group SX1297).

A compound according to Compound (2A) wherein R¹ represents a chlorine atom, Q represents Q2, L¹ represents an oxygen atom, and a combination of structure of G⁴ and substituents R^{X7}, R^{X8}, R^{X9} and R^{X10} applied to the structure of G⁴ represents any combination described in Combination K (hereinafter, referred to as Compound group SX1298).

A compound according to Compound (2A) wherein R¹ represents a methyl group, Q represents Q2, L¹ represents NH, and a combination of structure of G⁴ and substituents R^{X7}, R^{X8}, R^{X9} and R^{X10} applied to the structure of G⁴ represents any combination described in Combination K (hereinafter, referred to as Compound group SX1299).

A compound according to Compound (2A) wherein R¹ represents a chlorine atom, Q represents Q2, L¹ represents NH, and a combination of structure of G⁴ and substituents R^{X7}, R^{X8}, R^{X9} and R^{X10} applied to the structure of G⁴ represents any combination described in Combination K (hereinafter, referred to as Compound group SX1300).

A compound according to Compound (2A) wherein R¹ represents a methyl group, Q represents Q3, R³ represents a methoxy group, and a combination of structure of G⁴ and substituents R^{X7}, R^{X8}, R^{X9} and R^{X10} applied to the structure of G⁴ represents any combination described in Combination K (hereinafter, referred to as Compound group SX1301).

A compound according to Compound (2A) wherein R¹ represents a chlorine atom, Q represents Q3, R³ represents a methoxy group, and a combination of structure of G⁴ and substituents R^{X7}, R^{X8}, R^{X9} and R^{X10} applied to the structure of G⁴ represents any combination described in Combination K (hereinafter, referred to as Compound group SX1302).

A compound according to Compound (2A) wherein R¹ represents a methyl group, Q represents Q4, and a combination of structure of G⁴ and substituents R^{X7}, R^{X8}, R^{X9} and R^{X10} applied to the structure of G⁴ represents any combination described in Combination K (hereinafter, referred to as Compound group SX1303).

A compound according to Compound (2A) wherein R¹ represents a chlorine atom, Q represents Q4, and a combination of structure of G⁴ and substituents R^{X7}, R^{X8}, R^{X9} and R^{X10} applied to the structure of G⁴ represents any combination described in Combination K (hereinafter, referred to as Compound group SX1304).

A compound according to Compound (2A) wherein R¹ represents a methyl group, Q represents Q5, and a combination of structure of G⁴ and substituents R^{X7}, R^{X8}, R^{X9} and R^{X10} applied to the structure of G⁴ represents any combination described in Combination K (hereinafter, referred to as Compound group SX1305).

A compound according to Compound (2A) wherein R¹ represents a chlorine atom, Q represents Q5, and a combination of structure of G⁴ and substituents R^{X7}, R^{X8}, R^{X9} and R^{X10} applied to the structure of G⁴ represents any combination described in Combination K (hereinafter, referred to as Compound group SX1306).

A compound according to Compound (2A) wherein R¹ represents a methyl group, Q represents Q6, and a combination of structure of G⁴ and substituents R^{X7}, R^{X8}, R^{X9} and R^{X10} applied to the structure of G⁴ represents any combination described in Combination K (hereinafter, referred to as Compound group SX1307).

A compound according to Compound (2A) wherein R¹ represents a chlorine atom, Q represents Q6, and a combination of structure of G⁴ and substituents R^{X7}, R^{X8}, R^{X9} and R^{X10} applied to the structure of G⁴ represents any combination described in Combination K (hereinafter, referred to as Compound group SX1308).

A compound according to Compound (2A) wherein R¹ represents a methyl group, Q represents Q7, L² represents an oxygen atom, and a combination of structure of G⁴ and substituents R^{X7}, R^{X8}, R^{X9} and R^{X10} applied to the structure of G⁴ represents any combination described in Combination K (hereinafter, referred to as Compound group SX1309).

A compound according to Compound (2A) wherein R¹ represents a chlorine atom, Q represents Q7, L² represents an oxygen atom, and a combination of structure of G⁴ and substituents R^{X7}, R^{X8}, R^{X9} and R^{X10} applied to the structure of G⁴ represents any combination described in Combination K (hereinafter, referred to as Compound group SX1310).

A compound according to Compound (2A) wherein R¹ represents a methyl group, Q represents Q7, L² represents NH, and a combination of structure of G⁴ and substituents R^{X7}, R^{X8}, R^{X9} and R^{X10} applied to the structure of G⁴ represents any combination described in Combination K (hereinafter, referred to as Compound group SX1311).

A compound according to Compound (2A) wherein R¹ represents a chlorine atom, Q represents Q7, L² represents NH, and a combination of structure of G⁴ and substituents R^{X7}, R^{X8}, R^{X9} and R^{X10} applied to the structure of G⁴ represents any combination described in Combination K (hereinafter, referred to as Compound group SX1312).

A compound according to Compound (2A) wherein R¹ represents a methyl group, Q represents Q8A, and a combination of structure of G⁴ and substituents R^{X7}, R^{X8}, R^{X9} and R^{X10} applied to the structure of G⁴ represents any combination described in Combination K (hereinafter, referred to as Compound group SX1313).

A compound according to Compound (2A) wherein R¹ represents a chlorine atom, Q represents Q8A, and a combination of structure of G⁴ and substituents R^{X7}, R^{X8}, R^{X9} and R^{X10} applied to the structure of G⁴ represents any combination described in Combination K (hereinafter, referred to as Compound group SX1314).

A compound according to Compound (2A) wherein R¹ represents a methyl group, Q represents Q8D, and a combination of structure of G⁴ and substituents R^{X7}, R^{X8}, R^{X9} and R^{X10} applied to the structure of G⁴ represents any combination described in Combination K (hereinafter, referred to as Compound group SX1315).

A compound according to Compound (2A) wherein R¹ represents a chlorine atom, Q represents Q8D, and a combination of structure of G⁴ and substituents R^{X7}, R^{X8}, R^{X9} and R^{X10} applied to the structure of G⁴ represents any combination described in Combination K (hereinafter, referred to as Compound group SX1316).

A compound according to Compound (2A) wherein R¹ represents a methyl group, Q represents Q8J, and a combination of structure of G⁴ and substituents R^{X7}, R^{X8}, R^{X9} and R^{X10} applied to the structure of G⁴ represents any combination described in Combination K (hereinafter, referred to as Compound group SX1317).

A compound according to Compound (2A) wherein R¹ represents a chlorine atom, Q represents Q8J, and a combination of structure of G⁴ and substituents R^{X7}, R^{X8}, R^{X9} and R^{X10} applied to the structure of G⁴ represents any combination described in Combination K (hereinafter, referred to as Compound group SX1318).

A compound represented by formula (2C) (hereinafter, referred to as Compound (2C)):

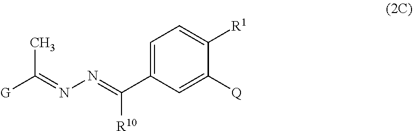

[wherein G represents any one of formulae G1 to G35.]
wherein R¹ represents a methyl group, R¹⁰ represents a methyl group, Q represents Q1, and a combination of structure of G and substituents R^{X7}, R^{X8}, R^{X9} and R^{X10} applied to the structure of G represents any combination described in Combination B (hereinafter, referred to as Compound group SX1319).

A compound according to Compound (2C) wherein R¹ represents a chlorine atom, R¹⁰ represents a methyl group, Q represents Q1, and a combination of structure of G and substituents R^{X7}, R^{X8}, R^{X9} and R^{X10} applied to the structure of G represents any combination described in Combination B (hereinafter, referred to as Compound group SX1320).

A compound according to Compound (2C) wherein R¹ represents a methyl group, R¹⁰ represents a methyl group, Q represents Q2, L¹ represents an oxygen atom, and a combination of structure of G and substituents R^{X7}, R^{X8}, R^{X9} and R^{X10} applied to the structure of G represents any combination described in Combination B (hereinafter, referred to as Compound group SX1321).

A compound according to Compound (2C) wherein R¹ represents a chlorine atom, R¹⁰ represents a methyl group, Q represents Q2, L¹ represents an oxygen atom, and a combination of structure of G and substituents R^{X7}, R^{X8}, R^{X9} and R^{X10} applied to the structure of G represents any combination described in Combination B (hereinafter, referred to as Compound group SX1322).

A compound according to Compound (2C) wherein R¹ represents a methyl group, R¹⁰ represents a methyl group, Q represents Q2, L¹ represents NH, and a combination of structure of G and substituents R^{X7}, R^{X8}, R^{X9} and R^{X10} applied to the structure of G represents any combination described in Combination B (hereinafter, referred to as Compound group SX1323).

A compound according to Compound (2C) wherein R¹ represents a chlorine atom, R¹⁰ represents a methyl group, Q represents Q2, L¹ represents NH, and a combination of structure of G and substituents R^{X7}, R^{X8}, R^{X9} and R^{X10} applied to the structure of G represents any combination described in Combination B (hereinafter, referred to as Compound group SX1324).

A compound according to Compound (2C) wherein R¹ represents a methyl group, R¹⁰ represents a methyl group, Q represents Q3, R³ represents a methoxy group, and a combination of structure of G and substituents R^{X7}, R^{X8}, R^{X9} and R^{X10} applied to the structure of G represents any combination described in Combination B (hereinafter, referred to as Compound group SX1325).

A compound according to Compound (2C) wherein R¹ represents a chlorine atom, R¹⁰ represents a methyl group, Q represents Q3, R³ represents a methoxy group, and a combination of structure of G and substituents R^{X7}, R^{X8}, R^{X9} and R^{X10} applied to the structure of G represents any combination described in Combination B (hereinafter, referred to as Compound group SX1326).

A compound according to Compound (2C) wherein R¹ represents a methyl group, R¹⁰ represents a methyl group, Q represents Q4, and a combination of structure of G and substituents R^{X7}, R^{X8}, R^{X9} and R^{X10} applied to the structure of G represents any combination described in Combination B (hereinafter, referred to as Compound group SX1327).

A compound according to Compound (2C) wherein R¹ represents a chlorine atom, R¹⁰ represents a methyl group, Q represents Q4, and a combination of structure of G and substituents R^{X7}, R^{X8}, R^{X9} and R^{X10} applied to the structure of G represents any combination described in Combination B (hereinafter, referred to as Compound group SX1328).

A compound according to Compound (2C) wherein R¹ represents a methyl group, R¹⁰ represents a methyl group, Q represents Q5, and a combination of structure of G and substituents R^{X7}, R^{X8}, R^{X9} and R^{X10} applied to the structure of G represents any combination described in Combination B (hereinafter, referred to as Compound group SX1329).

A compound according to Compound (2C) wherein R¹ represents a chlorine atom, R¹⁰ represents a methyl group, Q represents Q5, and a combination of structure of G and substituents R^{X7}, R^{X8}, R^{X9} and R^{X10} applied to the structure of G represents any combination described in Combination B (hereinafter, referred to as Compound group SX1330).

A compound according to Compound (2C) wherein R¹ represents a methyl group, R¹⁰ represents a methyl group, Q represents Q6, and a combination of structure of G and substituents R^{X7}, R^{X8}, R^{X9} and R^{X10} applied to the structure of G represents any combination described in Combination B (hereinafter, referred to as Compound group SX1331).

A compound according to Compound (2C) wherein R¹ represents a chlorine atom, R¹⁰ represents a methyl group, Q represents Q6, and a combination of structure of G and substituents R^{X7}, R^{X8}, R^{X9} and R^{X10} applied to the structure of G represents any combination described in Combination B (hereinafter, referred to as Compound group SX1332).

A compound according to Compound (2C) wherein R¹ represents a methyl group, R¹⁰ represents a methyl group, Q represents Q7, L² represents an oxygen atom, and a combination of structure of G and substituents R^{X7}, R^{X8}, R^{X9} and R^{X10} applied to the structure of G represents any combination described in Combination B (hereinafter, referred to as Compound group SX1333).

A compound according to Compound (2C) wherein R¹ represents a chlorine atom, R¹⁰ represents a methyl group, Q represents Q7, L² represents an oxygen atom, and a combination of structure of G and substituents R^{X7}, R^{X8}, R^{X9} and R^{X10} applied to the structure of G represents any combination described in Combination B (hereinafter, referred to as Compound group SX1334).

A compound according to Compound (2C) wherein R¹ represents a methyl group, R¹⁰ represents a methyl group, Q represents Q7, L² represents NH, and a combination of structure of G and substituents R^{X7}, R^{X8}, R^{X9} and R^{X10} applied to the structure of G represents any combination described in Combination B (hereinafter, referred to as Compound group SX1335).

A compound according to Compound (2C) wherein R¹ represents a chlorine atom, R¹⁰ represents a methyl group, Q represents Q7, L² represents NH, and a combination of structure of G and substituents R^{X7}, R^{X8}, R^{X9} and R^{X10} applied to the structure of G represents any combination described in Combination B (hereinafter, referred to as Compound group SX1336).

A compound according to Compound (2C) wherein R¹ represents a methyl group, R¹⁰ represents a methyl group, Q represents Q8A, and a combination of structure of G and substituents R^{X7}, R^{X8}, R^{X9} and R^{X10} applied to the structure of G represents any combination described in Combination B (hereinafter, referred to as Compound group SX1337).

A compound according to Compound (2C) wherein R¹ represents a chlorine atom, R¹⁰ represents a methyl group, Q represents Q8A, and a combination of structure of G and substituents R^{X7}, R^{X8}, R^{X9} and R^{X10} applied to the structure of G represents any combination described in Combination B (hereinafter, referred to as Compound group SX1338).

A compound according to Compound (2C) wherein R¹ represents a methyl group, R¹⁰ represents a methyl group, Q represents Q8D, and a combination of structure of G and substituents R^{X7}, R^{X8}, R^{X9} and R^{X10} applied to the structure of G represents any combination described in Combination B (hereinafter, referred to as Compound group SX1339).

A compound according to Compound (2C) wherein R¹ represents a chlorine atom, R¹⁰ represents a methyl group, Q represents Q8D, and a combination of structure of G and substituents R^{X7}, R^{X8}, R^{X9} and R^{X10} applied to the structure of G represents any combination described in Combination B (hereinafter, referred to as Compound group SX1340).

A compound according to Compound (2C) wherein R¹ represents a methyl group, R¹⁰ represents a methyl group, Q represents Q8J, and a combination of structure of G and substituents R^{X7}, R^{X8}, R^{X9} and R^{X10} applied to the structure of G represents any combination described in Combination B (hereinafter, referred to as Compound group SX1341).

A compound according to Compound (2C) wherein R¹ represents a chlorine atom, R¹⁰ represents a methyl group, Q represents Q8J, and a combination of structure of G and substituents R^{X7}, R^{X8}, R^{X9} and R^{X10} applied to the structure of G represents any combination described in Combination B (hereinafter, referred to as Compound group SX1342).

A compound according to Compound (2C) wherein R¹ represents a methyl group, R¹⁰ represents an ethyl group, Q represents Q1, and a combination of structure of G and substituents R^{X7}, R^{X8}, R^{X9} and R^{X10} applied to the structure of G represents any combination described in Combination B (hereinafter, referred to as Compound group SX1343).

A compound according to Compound (2C) wherein R¹ represents a chlorine atom, R¹⁰ represents an ethyl group, Q represents Q1, and a combination of structure of G and substituents R^{X7}, R^{X8}, R^{X9} and R^{X10} applied to the structure of G represents any combination described in Combination B (hereinafter, referred to as Compound group SX1344).

A compound according to Compound (2C) wherein R¹ represents a methyl group, R¹⁰ represents an ethyl group, Q represents Q2, L¹ represents an oxygen atom, and a combination of structure of G and substituents R^{X7}, R^{X8}, R^{X9} and R^{X10} applied to the structure of G represents any combination described in Combination B (hereinafter, referred to as Compound group SX1345).

A compound according to Compound (2C) wherein R¹ represents a chlorine atom, R¹⁰ represents an ethyl group, Q represents Q2, L¹ represents an oxygen atom, and a combination of structure of G and substituents R^{X7}, R^{X8}, R^{X9} and R^{X10} applied to the structure of G represents any combination described in Combination B (hereinafter, referred to as Compound group SX1346).

A compound according to Compound (2C) wherein R¹ represents a methyl group, R¹⁰ represents an ethyl group, Q represents Q2, L¹ represents NH, and a combination of structure of G and substituents R^{X7}, R^{X8}, R^{X9} and R^{X10} applied to the structure of G represents any combination described in Combination B (hereinafter, referred to as Compound group SX1347).

A compound according to Compound (2C) wherein R¹ represents a chlorine atom, R¹⁰ represents an ethyl group, Q represents Q2, L¹ represents NH, and a combination of structure of G and substituents R^{X7}, R^{X8}, R^{X9} and R^{X10} applied to the structure of G represents any combination described in Combination B (hereinafter, referred to as Compound group SX1348).

A compound according to Compound (2C) wherein R¹ represents a methyl group, R¹⁰ represents an ethyl group, Q represents Q3, R³ represents a methoxy group, and a combination of structure of G and substituents R^{X7}, Rx, R^{X9} and R^{X10} applied to the structure of G represents any combination described in Combination B (hereinafter, referred to as Compound group SX1349).

A compound according to Compound (2C) wherein R¹ represents a chlorine atom, R¹⁰ represents an ethyl group, Q represents Q3, R³ represents a methoxy group, and a combination of structure of G and substituents R^{X7}, Rx, R^{X9} and R^{X10} applied to the structure of G represents any combination described in Combination B (hereinafter, referred to as Compound group SX1350).

A compound according to Compound (2C) wherein R¹ represents a methyl group, R¹⁰ represents an ethyl group, Q represents Q4, and a combination of structure of G and substituents R^{X7}, R^{X8}, R^{X9} and R^{X10} applied to the structure of G represents any combination described in Combination B (hereinafter, referred to as Compound group SX1351).

A compound according to Compound (2C) wherein R¹ represents a chlorine atom, R¹⁰ represents an ethyl group, Q represents Q4, and a combination of structure of G and substituents R^{X7}, R^{X8}, R^{X9} and R^{X10} applied to the structure of G represents any combination described in Combination B (hereinafter, referred to as Compound group SX1352).

A compound according to Compound (2C) wherein R¹ represents a methyl group, R¹⁰ represents an ethyl group, Q represents Q5, and a combination of structure of G and substituents R^{X7}, R^{X8}, R^{X9} and R^{X10} applied to the structure of G represents any combination described in Combination B (hereinafter, referred to as Compound group SX1353).

A compound according to Compound (2C) wherein R¹ represents a chlorine atom, R¹⁰ represents an ethyl group, Q represents Q5, and a combination of structure of G and substituents R^{X7}, R^{X8}, R^{X9} and R^{X10} applied to the structure of G represents any combination described in Combination B (hereinafter, referred to as Compound group SX1354).

A compound according to Compound (2C) wherein R¹ represents a methyl group, R¹⁰ represents an ethyl group, Q represents Q6, and a combination of structure of G and substituents R^{X7}, R^{X8}, R^{X9} and R^{X10} applied to the structure of G represents any combination described in Combination B (hereinafter, referred to as Compound group SX1355).

A compound according to Compound (2C) wherein R¹ represents a chlorine atom, R¹⁰ represents an ethyl group, Q represents Q6, and a combination of structure of G and substituents R^{X7}, R^{X8}, R^{X9} and R^{X10} applied to the structure of G represents any combination described in Combination B (hereinafter, referred to as Compound group SX1356).

A compound according to Compound (2C) wherein R¹ represents a methyl group, R¹⁰ represents an ethyl group, Q represents Q7, L² represents an oxygen atom, and a combination of structure of G and substituents R^{X7}, R^{X8}, R^{X9} and R^{X10} applied to the structure of G represents any combination described in Combination B (hereinafter, referred to as Compound group SX1357).

A compound according to Compound (2C) wherein R¹ represents a chlorine atom, R¹⁰ represents an ethyl group, Q represents Q7, L² represents an oxygen atom, and a combination of structure of G and substituents R^{X7}, R^{X8}, R^{X9} and R^{X10} applied to the structure of G represents any combination described in Combination B (hereinafter, referred to as Compound group SX1358).

A compound according to Compound (2C) wherein R¹ represents a methyl group, R¹⁰ represents an ethyl group, Q represents Q7, L² represents NH, and a combination of structure of G and substituents R^{X7}, R^{X8}, R^{X9} and R^{X10} applied to the structure of G represents any combination described in Combination B (hereinafter, referred to as Compound group SX1359).

A compound according to Compound (2C) wherein R¹ represents a chlorine atom, R¹⁰ represents an ethyl group, Q represents Q7, L² represents NH, and a combination of structure of G and substituents R^{X7}, R^{X8}, R^{X9} and R^{X10} applied to the structure of G represents any combination described in Combination B (hereinafter, referred to as Compound group SX1360).

A compound according to Compound (2C) wherein R¹ represents a methyl group, R¹⁰ represents an ethyl group, Q represents Q8A, and a combination of structure of G and substituents R^{X7}, R^{X8}, R^{X9} and R^{X10} applied to the structure of G represents any combination described in Combination B (hereinafter, referred to as Compound group SX1361).

A compound according to Compound (2C) wherein R¹ represents a chlorine atom, R¹⁰ represents an ethyl group, Q represents Q8A, and a combination of structure of G and substituents R^{X7}, R^{X8}, R^{X9} and R^{X10} applied to the structure of G represents any combination described in Combination B (hereinafter, referred to as Compound group SX1362).

A compound according to Compound (2C) wherein R¹ represents a methyl group, R¹⁰ represents an ethyl group, Q represents Q8D, and a combination of structure of G and substituents R^{X7}, R^{X8}, R^{X9} and R^{X10} applied to the structure of G represents any combination described in Combination B (hereinafter, referred to as Compound group SX1363).

A compound according to Compound (2C) wherein R¹ represents a chlorine atom, R¹⁰ represents an ethyl group, Q represents Q8D, and a combination of structure of G and substituents R^{X7}, R^{X8}, R^{X9} and R^{X10} applied to the structure of G represents any combination described in Combination B (hereinafter, referred to as Compound group SX1364).

A compound according to Compound (2C) wherein R¹ represents a methyl group, R¹⁰ represents an ethyl group, Q represents Q8J, and a combination of structure of G and substituents R^{X7}, R^{X8}, R^{X9} and R^{X10} applied to the structure of G represents any combination described in Combination B (hereinafter, referred to as Compound group SX1365).

A compound according to Compound (2C) wherein R¹ represents a chlorine atom, R¹⁰ represents an ethyl group, Q represents Q8J, and a combination of structure of G and substituents R^{X7}, R^{X8}, R^{X9} and R^{X10} applied to the structure of G represents any combination described in Combination B (hereinafter, referred to as Compound group SX1366).

A compound according to Compound (2C) wherein R¹ represents a methyl group, R¹⁰ represents a cyclopropyl group, Q represents Q1, and a combination of structure of G and substituents R^{X7}, R^{X8}, R^{X9} and R^{X10} applied to the structure of G represents any combination described in Combination B (hereinafter, referred to as Compound group SX1367).

A compound according to Compound (2C) wherein R¹ represents a chlorine atom, R¹⁰ represents a cyclopropyl group, Q represents Q1, and a combination of structure of G and substituents R^{X7}, R^{X8}, R^{X9} and R^{X10} applied to the structure of G represents any combination described in Combination B (hereinafter, referred to as Compound group SX1368).

A compound according to Compound (2C) wherein R¹ represents a methyl group, R¹⁰ represents a cyclopropyl group, Q represents Q2, L¹ represents an oxygen atom, and a combination of structure of G and substituents R^{X7}, R^{X8}, R^{X9} and R^{X10} applied to the structure of G represents any combination described in Combination B (hereinafter, referred to as Compound group SX1369).

A compound according to Compound (2C) wherein R¹ represents a chlorine atom, R¹⁰ represents a cyclopropyl group, Q represents Q2, L¹ represents an oxygen atom, and a combination of structure of G and substituents R^{X7}, R^{X8}, R^{X9} and R^{X10} applied to the structure of G represents any combination described in Combination B (hereinafter, referred to as Compound group SX1370).

A compound according to Compound (2C) wherein R¹ represents a methyl group, R¹⁰ represents a cyclopropyl group, Q represents Q2, L¹ represents NH, and a combination of structure of G and substituents R^{X7}, R^{X8}, R^{X9} and R^{X10} applied to the structure of G represents any combination described in Combination B (hereinafter, referred to as Compound group SX1371).

A compound according to Compound (2C) wherein R¹ represents a chlorine atom, R¹⁰ represents a cyclopropyl group, Q represents Q2, L¹ represents NH, and a combination of structure of G and substituents R^{X7}, R^{X8}, R^{X9} and R^{X10} applied to the structure of G represents any combination described in Combination B (hereinafter, referred to as Compound group SX1372).

A compound according to Compound (2C) wherein R¹ represents a methyl group, R¹⁰ represents a cyclopropyl group, Q represents Q3, R³ represents a methoxy group, and a combination of structure of G and substituents R^{X7}, R^{X8}, R^{X9} and R^{X10} applied to the structure of G represents any combination described in Combination B (hereinafter, referred to as Compound group SX1373).

A compound according to Compound (2C) wherein R¹ represents a chlorine atom, R¹⁰ represents a cyclopropyl group, Q represents Q3, R³ represents a methoxy group, and a combination of structure of G and substituents R^{X7}, R^{X8}, R^{X9} and R^{X10} applied to the structure of G represents any combination described in Combination B (hereinafter, referred to as Compound group SX1374).

A compound according to Compound (2C) wherein R¹ represents a methyl group, R¹⁰ represents a cyclopropyl group, Q represents Q4, and a combination of structure of G and substituents R^{X7}, R^{X8}, R^{X9} and R^{X10} applied to the structure of G represents any combination described in Combination B (hereinafter, referred to as Compound group SX1375).

A compound according to Compound (2C) wherein R¹ represents a chlorine atom, R¹⁰ represents a cyclopropyl group, Q represents Q4, and a combination of structure of G and substituents R^{X7}, R^{X8}, R^{X9} and R^{X10} applied to the structure of G represents any combination described in Combination B (hereinafter, referred to as Compound group SX1376).

A compound according to Compound (2C) wherein R¹ represents a methyl group, R¹⁰ represents a cyclopropyl group, Q represents Q5, and a combination of structure of G and substituents R^{X7}, R^{X8}, R^{X9} and R^{X10} applied to the structure of G represents any combination described in Combination B (hereinafter, referred to as Compound group SX1377).

A compound according to Compound (2C) wherein R¹ represents a chlorine atom, R¹⁰ represents a cyclopropyl group, Q represents Q5, and a combination of structure of G and substituents R^{X7}, R^{X8}, R^{X9} and R^{X10} applied to the structure of G represents any combination described in Combination B (hereinafter, referred to as Compound group SX1378).

A compound according to Compound (2C) wherein R¹ represents a methyl group, R¹⁰ represents a cyclopropyl group, Q represents Q6, and a combination of structure of G and substituents R^{X7}, R^{X8}, R^{X9} and R^{X10} applied to the structure of G represents any combination described in Combination B (hereinafter, referred to as Compound group SX1379).

A compound according to Compound (2C) wherein R¹ represents a chlorine atom, R¹⁰ represents a cyclopropyl group, Q represents Q6, and a combination of structure of G and substituents R^{X7}, R^{X8}, R^{X9} and R^{X10} applied to the structure of G represents any combination described in Combination B (hereinafter, referred to as Compound group SX1380).

A compound according to Compound (2C) wherein R¹ represents a methyl group, R¹⁰ represents a cyclopropyl group, Q represents Q7, L² represents an oxygen atom, and a combination of structure of G and substituents R^{X7}, R^{X8}, R^{X9} and R^{X10} applied to the structure of G represents any combination described in Combination B (hereinafter, referred to as compound group SX1381).

A compound according to Compound (2C) wherein R¹ represents a chlorine atom, R¹⁰ represents a cyclopropyl group, Q represents Q7, L² represents an oxygen atom, and a combination of structure of G and substituents R^{X7}, R^{X8}, R^{X9} and R^{X10} applied to the structure of G represents any combination described in Combination B (hereinafter, referred to as Compound group SX1382).

A compound according to Compound (2C) wherein R¹ represents a methyl group, R¹⁰ represents a cyclopropyl group, Q represents Q7, L² represents NH, and a combination of structure of G and substituents R^{X7}, R^{X8}, R^{X9} and R^{X10} applied to the structure of G represents any combination described in Combination B (hereinafter, referred to as Compound group SX1383).

A compound according to Compound (2C) wherein R¹ represents a chlorine atom, R¹⁰ represents a cyclopropyl group, Q represents Q7, L² represents NH, and a combination of structure of G and substituents R^{X7}, R^{X8}, R^{X9} and R^{X10} applied to the structure of G represents any combination described in Combination B (hereinafter, referred to as Compound group SX1384).

A compound according to Compound (2C) wherein R¹ represents a methyl group, R¹⁰ represents a cyclopropyl group, Q represents Q8A, and a combination of structure of G and substituents R^{X7}, R^{X8}, R^{X9} and R^{X10} applied to the structure of G represents any combination described in Combination B (hereinafter, referred to as Compound group SX1385).

A compound according to Compound (2C) wherein R¹ represents a chlorine atom, R¹⁰ represents a cyclopropyl group, Q represents Q8A, and a combination of structure of G and substituents R^{X7}, R^{X8}, R^{X9} and R^{X10} applied to the structure of G represents any combination described in Combination B (hereinafter, referred to as Compound group SX1386).

A compound according to Compound (2C) wherein R¹ represents a methyl group, R¹⁰ represents a cyclopropyl group, Q represents Q8D, and a combination of structure of G and substituents R^{X7}, R^{X8}, R^{X9} and R^{X10} applied to the structure of G represents any combination described in Combination B (hereinafter, referred to as Compound group SX1387).

A compound according to Compound (2C) wherein R¹ represents a chlorine atom, R¹⁰ represents a cyclopropyl group, Q represents Q8D, and a combination of structure of G and substituents R^{X7}, R^{X8}, R^{X9} and R^{X10} applied to the structure of G represents any combination described in Combination B (hereinafter, referred to as Compound group SX1388).

A compound according to Compound (2C) wherein R¹ represents a methyl group, R¹⁰ represents a cyclopropyl group, Q represents Q8J, and a combination of structure of G and substituents R^{X7}, R^{X8}, R^{X9} and R^{X10} applied to the structure of G represents any combination described in Combination B (hereinafter, referred to as Compound group SX1389).

A compound according to Compound (2C) wherein R¹ represents a chlorine atom, R¹⁰ represents a cyclopropyl group, Q represents Q8J, and a combination of structure of G and substituents R^{X7}, R^{X8}, R^{X9} and R^{X10} applied to the structure of G represents any combination described in Combination B (hereinafter, referred to as Compound group SX1390).

A compound according to Compound (2F) wherein R¹ represents a methyl group, Q represents Q1, R⁷ represents a methyl group, and a combination of R^{X2}, R^{X3}, R^{X4}, R^{X5} and R^{X6} represents any combination described in Combination A (hereinafter, referred to as Compound group SX1391).

A compound according to Compound (2F) wherein R¹ represents a chlorine atom, Q represents Q1, R⁷ represents a methyl group, and a combination of R^{X2}, R^{X3}, R^{X4}, R^{X5} and R^{X6} represents any combination described in Combination A (hereinafter, referred to as Compound group SX1392).

A compound according to Compound (2F) wherein R¹ represents a methyl group, Q represents Q2, L¹ represents an oxygen atom, R⁷ represents a methyl group, and a combination of R^{X2}, R^{X3}, R^{X4}, R^{X5} and R^{X6} represents any combination described in Combination A (hereinafter, referred to as Compound group SX1393).

A compound according to Compound (2F) wherein R¹ represents a chlorine atom, Q represents Q2, L¹ represents an oxygen atom, R⁷ represents a methyl group, and a combination of R^{X2}, R^{X3}, R^{X4}, R^{X5} and R^{X6} represents any combination described in Combination A (hereinafter, referred to as Compound group SX1394).

A compound according to Compound (2F) wherein R¹ represents a methyl group, Q represents Q2, L¹ represents NH, R⁷ represents a methyl group, and a combination of R^{X2}, R^{X3}, R^{X4}, R^{X5} and R^{X6} represents any combination described in Combination A (hereinafter, referred to as Compound group SX1395).

A compound according to Compound (2F) wherein R¹ represents a chlorine atom, Q represents Q2, L¹ represents NH, R⁷ represents a methyl group, and a combination of R^{X2}, R^{X3}, R^{X4}, R^{X5} and R^{X6} represents any combination described in Combination A (hereinafter, referred to as Compound group SX1396).

A compound according to Compound (2F) wherein R¹ represents a methyl group, Q represents Q3, R³ represents a methoxy group, R⁷ represents a methyl group, and a combination of R^{X2}, R^{X3}, R^{X4}, R^{X5} and R^{X6} represents any combination described in Combination A (hereinafter, referred to as Compound group SX1397).

A compound according to Compound (2F) wherein R¹ represents a chlorine atom, Q represents Q3, R³ represents a methoxy group, R⁷ represents a methyl group, and a combination of R^{X2}, R^{X3}, R^{X4}, R^{X5} and R^{X6} represents any combination described in Combination A (hereinafter, referred to as Compound group SX1398).

A compound according to Compound (2F) wherein R¹ represents a methyl group, Q represents Q4, R⁷ represents a methyl group, and a combination of R^{X2}, R^{X3}, R^{X4}, R^{X5} and R^{X6} represents any combination described in Combination A (hereinafter, referred to as Compound group SX1399).

A compound according to Compound (2F) wherein R¹ represents a chlorine atom, Q represents Q4, R⁷ represents a methyl group, and a combination of R^{X2}, R^{X3}, R^{X4}, R^{X5} and R^{X6} represents any combination described in Combination A (hereinafter, referred to as Compound group SX1400).

A compound according to Compound (2F) wherein R¹ represents a methyl group, Q represents Q5, R⁷ represents a methyl group, and a combination of R^{X2}, R^{X3}, R^{X4}, R^{X5} and R^{X6} represents any combination described in Combination A (hereinafter, referred to as Compound group SX1401).

A compound according to Compound (2F) wherein R¹ represents a chlorine atom, Q represents Q5, R⁷ represents a methyl group, and a combination of R^{X2}, R^{X3}, R^{X4}, R^{X5} and R^{X6} represents any combination described in Combination A (hereinafter, referred to as Compound group SX1402).

A compound according to Compound (2F) wherein R¹ represents a methyl group, Q represents Q6, R⁷ represents a methyl group, and a combination of R^{X2}, R^{X3}, R^{X4}, R^{X5} and R^{X6} represents any combination described in Combination A (hereinafter, referred to as Compound group SX1403).

A compound according to Compound (2F) wherein R¹ represents a chlorine atom, Q represents Q6, R⁷ represents a methyl group, and a combination of R^{X2}, R^{X3}, R^{X4}, R^{X5} and R^{X6} represents any combination described in Combination A (hereinafter, referred to as Compound group SX1404).

A compound according to Compound (2F) wherein R¹ represents a methyl group, Q represents Q7, L² represents an oxygen atom, R⁷ represents a methyl group, and a combination of R^{X2}, R^{X3}, R^{X4}, R^{X5} and R^{X6} represents any combination described in Combination A (hereinafter, referred to as Compound group SX1405).

A compound according to Compound (2F) wherein R¹ represents a chlorine atom, Q represents Q7, L² represents an oxygen atom, R⁷ represents a methyl group, and a combination of R^{X2}, R^{X3}, R^{X4}, R^{X5} and R^{X6} represents any combination described in Combination A (hereinafter, referred to as Compound group SX1406).

A compound according to Compound (2F) wherein R¹ represents a methyl group, Q represents Q7, L² represents NH, R⁷ represents a methyl group, and a combination of R^{X2}, R^{X3}, R^{X4}, R^{X5} and R^{X6} represents any combination described in Combination A (hereinafter, referred to as Compound group SX1407).

A compound according to Compound (2F) wherein R¹ represents a chlorine atom, Q represents Q7, L² represents NH, R⁷ represents a methyl group, and a combination of R^{X2}, R^{X3}, R^{X4}, R^{X5} and R^{X6} represents any combination described in Combination A (hereinafter, referred to as Compound group SX1408).

A compound according to Compound (2F) wherein R¹ represents a methyl group, Q represents Q8A, R⁷ represents a methyl group, and a combination of R^{X2}, R^{X3}, R^{X4}, R^{X5} and R^{X6} represents any combination described in Combination A (hereinafter, referred to as Compound group SX1409).

A compound according to Compound (2F) wherein R¹ represents a chlorine atom, Q represents Q8A, R⁷ represents a methyl group, and a combination of R^{X2}, R^{X3}, R^{X4}, R^{X5} and R^{X6} represents any combination described in Combination A (hereinafter, referred to as Compound group SX1410).

A compound according to Compound (2F) wherein R¹ represents a methyl group, Q represents Q8D, R⁷ represents a methyl group, and a combination of R^{X2}, R^{X3}, R^{X4}, R^{X5} and R^{X6} represents any combination described in Combination A (hereinafter, referred to as Compound group SX1411).

A compound according to Compound (2F) wherein R¹ represents a chlorine atom, Q represents Q8D, R⁷ represents a methyl group, and a combination of R^{X2}, R^{X3}, R^{X4}, R^{X5} and R^{X6} represents any combination described in Combination A (hereinafter, referred to as Compound group SX1412).

A compound according to Compound (2F) wherein R¹ represents a methyl group, Q represents Q8J, R⁷ represents a methyl group, and a combination of R^{X2}, R^{X3}, R^{X4}, R^{X5} and R^{X6} represents any combination described in Combination A (hereinafter, referred to as Compound group SX1413).

A compound according to Compound (2F) wherein R¹ represents a chlorine atom, Q represents Q8J, R⁷ represents a methyl group, and a combination of R^{X2}, R^{X3}, R^{X4}, R^{X5} and R^{X6} represents any combination described in Combination A (hereinafter, referred to as Compound group SX1414).

A compound according to Compound (2F) wherein R¹ represents a methyl group, Q represents Q1, R⁷ represents an ethyl group, and a combination of R^{X2}, R^{X3}, R^{X4}, R^{X5} and R^{X6} represents any combination described in Combination A (hereinafter, referred to as Compound group SX1415).

A compound according to Compound (2F) wherein R¹ represents a chlorine atom, Q represents Q1, R⁷ represents an ethyl group, and a combination of R^{X2}, R^{X3}, R^{X4}, R^{X5} and R^{X6} represents any combination described in Combination A (hereinafter, referred to as Compound group SX1416).

A compound according to Compound (2F) wherein R¹ represents a methyl group, Q represents Q2, L¹ represents an oxygen atom, R⁷ represents an ethyl group, and a combination of R^{X2}, R^{X3}, R^{X4}, R^{X5} and R^{X6} represents any combination described in Combination A (hereinafter, referred to as Compound group SX1417).

A compound according to Compound (2F) wherein R¹ represents a chlorine atom, Q represents Q2, L¹ represents an oxygen atom, R⁷ represents an ethyl group, and a combination of R^{X2}, R^{X3}, R^{X4}, R^{X5} and R^{X6} represents any combination described in Combination A (hereinafter, referred to as Compound group SX1418).

A compound according to Compound (2F) wherein R¹ represents a methyl group, Q represents Q2, L¹ represents NH, R⁷ represents an ethyl group, and a combination of R^{X2}, R^{X3}, R^{X4}, R^{X5} and R^{X6} represents any combination described in Combination A (hereinafter, referred to as Compound group SX1419).

A compound according to Compound (2F) wherein R¹ represents a chlorine atom, Q represents Q2, L¹ represents NH, R⁷ represents an ethyl group, and a combination of R^{X2}, R^{X3}, R^{X4}, R^{X5} and R^{X6} represents any combination described in Combination A (hereinafter, referred to as Compound group SX1420).

A compound according to Compound (2F) wherein R¹ represents a methyl group, Q represents Q3, R³ represents a methoxy group, R⁷ represents an ethyl group, and a combination of R^{X2}, R^{X3}, R^{X4}, R^{X5} and R^{X6} represents any combination described in Combination A (hereinafter, referred to as Compound group SX1421).

A compound according to Compound (2F) wherein R¹ represents a chlorine atom, Q represents Q3, R³ represents a methoxy group, R⁷ represents an ethyl group, and a combination of R^{X2}, R^{X3}, R^{X4}, R^{X5} and R^{X6} represents any combination described in Combination A (hereinafter, referred to as Compound group SX1422).

A compound according to Compound (2F) wherein R¹ represents a methyl group, Q represents Q4, R⁷ represents an ethyl group, and a combination of R^{X2}, R^{X3}, R^{X4}, R^{X5} and R^{X6} represents any combination described in Combination A (hereinafter, referred to as Compound group SX1423).

A compound according to Compound (2F) wherein R¹ represents a chlorine atom, Q represents Q4, R⁷ represents an ethyl group, and a combination of R^{X2}, R^{X3}, R^{X4}, R^{X5} and R^{X6} represents any combination described in Combination A (hereinafter, referred to as Compound group SX1424).

A compound according to Compound (2F) wherein R¹ represents a methyl group, Q represents Q5, R⁷ represents an ethyl group, and a combination of R^{X2}, R^{X3}, R^{X4}, R^{X5} and R^{X6} represents any combination described in Combination A (hereinafter, referred to as Compound group SX1425).

A compound according to Compound (2F) wherein R¹ represents a chlorine atom, Q represents Q5, R⁷ represents an ethyl group, and a combination of R^{X2}, R^{X3}, R^{X4}, R^{X5} and R^{X6} represents any combination described in Combination A (hereinafter, referred to as Compound group SX1426).

A compound according to Compound (2F) wherein R¹ represents a methyl group, Q represents Q6, R⁷ represents an ethyl group, and a combination of R^{X2}, R^{X3}, R^{X4}, R^{X5} and R^{X6} represents any combination described in Combination A (hereinafter, referred to as Compound group SX1427).

A compound according to Compound (2F) wherein R¹ represents a chlorine atom, Q represents Q6, R⁷ represents an ethyl group, and a combination of R^{X2}, R^{X3}, R^{X4}, R^{X5} and R^{X6} represents any combination described in Combination A (hereinafter, referred to as Compound group SX1428).

A compound according to Compound (2F) wherein R¹ represents a methyl group, Q represents Q7, L² represents an oxygen atom, R⁷ represents an ethyl group, and a combination of R^{X2}, R^{X3}, R^{X4}, R^{X5} and R^{X6} represents any combination described in Combination A (hereinafter, referred to as Compound group SX1429).

A compound according to Compound (2F) wherein R¹ represents a chlorine atom, Q represents Q7, L² represents an oxygen atom, R⁷ represents an ethyl group, and a combination of R^{X2}, R^{X3}, R^{X4}, R^{X5} and R^{X6} represents any combination described in Combination A (hereinafter, referred to as Compound group SX1430).

A compound according to Compound (2F) wherein R¹ represents a methyl group, Q represents Q7, L² represents NH, R⁷ represents an ethyl group, and a combination of R^{X2}, R^{X3}, R^{X4}, R^{X5} and R^{X6} represents any combination described in Combination A (hereinafter, referred to as Compound group SX1431).

A compound according to Compound (2F) wherein R¹ represents a chlorine atom, Q represents Q7, L² represents NH, R⁷ represents an ethyl group, and a combination of R^{X2}, R^{X3}, R^{X4}, R^{X5} and R^{X6} represents any combination described in Combination A (hereinafter, referred to as Compound group SX1432).

A compound according to Compound (2F) wherein R¹ represents a methyl group, Q represents Q8A, R⁷ represents an ethyl group, and a combination of R^{X2}, R^{X3}, R^{X4}, R^{X5} and R^{X6} represents any combination described in Combination A (hereinafter, referred to as Compound group SX1433).

A compound according to Compound (2F) wherein R¹ represents a chlorine atom, Q represents Q8A, R⁷ represents an ethyl group, and a combination of R^{X2}, R^{X3}, R^{X4}, R^{X5} and R^{X6} represents any combination described in Combination A (hereinafter, referred to as Compound group SX1434).

A compound according to Compound (2F) wherein R¹ represents a methyl group, Q represents Q8D, R⁷ represents an ethyl group, and a combination of R^{X2}, R^{X3}, R^{X4}, R^{X5} and R^{X6} represents any combination described in Combination A (hereinafter, referred to as Compound group SX1435).

A compound according to Compound (2F) wherein R¹ represents a chlorine atom, Q represents Q8D, R⁷ represents an ethyl group, and a combination of R^{X2}, R^{X3}, R^{X4}, R^{X5} and R^{X6} represents any combination described in Combination A (hereinafter, referred to as Compound group SX1436).

A compound according to Compound (2F) wherein R¹ represents a methyl group, Q represents Q8J, R⁷ represents an ethyl group, and a combination of R^{X2}, R^{X3}, R^{X4}, R^{X5} and R^{X6} represents any combination described in Combination A (hereinafter, referred to as Compound group SX1437).

A compound according to Compound (2F) wherein R¹ represents a chlorine atom, Q represents Q8J, R⁷ represents an ethyl group, and a combination of R^{X2}, R^{X3}, R^{X4}, R^{X5} and R^{X6} represents any combination described in Combination A (hereinafter, referred to as Compound group SX1438).

A compound according to Compound (1E) wherein R¹ represents an ethyl group, Q represents Q1, and a combination of R^{X2}, R^{X3}, R^{X4}, R^{X5} and R^{X6} represents any combination described in Combination A (hereinafter, referred to as Compound group SX1439).

A compound according to Compound (1E) wherein R¹ represents a fluorine atom, Q represents Q1, and a combination of R^{X2}, R^{X3}, R^{X4}, R^{X5} and R^{X6} represents any combination described in Combination A (hereinafter, referred to as Compound group SX1440).

A compound according to Compound (1E) wherein R¹ represents a cyclopropyl group, Q represents Q1, and a combination of R^{X2}, R^{X3}, R^{X4}, R^{X5} and R^{X6} represents any combination described in Combination A (hereinafter, referred to as Compound group SX1441).

A compound according to Compound (1E) wherein R¹ represents a methyl group, Q represents Q1, and a combination of R^{X2}, R^{X3}, R^{X4}, R^{X5} and R^{X6} represents any combination described in Combination G (hereinafter, referred to as Compound group SX1442).

Combination G consists of substituent numbers ZG1 to ZG170. Substituent numbers ZG1 to ZG170 represent combinations of substituents R^{X2}, R^{X3}, R^{X4}, R^{X5} and R^{X6} according to Compound (1E). And hereinafter, they are referred to as [substituent number; R^{X2}, R^{X3}, R^{X4}, R^{X5}, R^{X6}]. For example, the substituent number ZG2 refers to a combination wherein R^{X2} represents a trifluoromethoxy group, and each of R^{X3}, R^{X4}, R^{X5} and R^{X6} represents a hydrogen atom.
Combination G: [ZG1; OMe, H, H, H, H], [ZG2; OCF₃, H, H, H, H], [ZG3; OCHF₂, H, H, H, H], [Z G4;SMe,H,H,H,H], [ZG5;SCF₃,H,H,H,H], [ZG6;SCHF₂,H,H,H,H], [ZG7;F,H,H,H,H], [ZG8;Cl,H,H,H,H], [ZG9;Br,H,H,H,H], [ZG10;I,H,H,H,H], [ZG11;CN,H,H,H,H], [ZG12;Et,H,H,H,H], [ZG13;Pr,H,H,H,H], [ZG14;i-Pr,H,H,H,H], [ZG15;t-Bu,H,H,H,H], [ZG16;Ph,H,H,H,H], [ZG17;HCl,H,H,H], [ZG18;H,CHF₂,H,H,H], [ZG19;H,CF₃,H,H,H], [ZG20;H,Et,H,H,H], [ZG21;H,Pr,H,H,H], [ZG22;H,i-Pr,H,H,H], [ZG23;H,Ph,H,H,H], [ZG24;H,H,OMe,H,H], [ZG25;H,H,OC HF₂,H,H], [ZG26;H,H,F,H,H], [ZG27;H,H,Cl,H,H], [ZG28;H,H,Br,H, H], [ZG29;H,H, I,H,H], [ZG30;H,H,CN,H,H], [ZG31;H,H,CHF₂,H,H], [ZG32;H,H,CF₃,H,H], [ZG33;H,H,Me,H,H], [ZG34;H,H,Et,H,H], [ZG35;H,H,Pr,H,H], [ZG36;H,H,i-Pr,H,H], [ZG37;H,H,Ph,H,H], [ZG38;F,F,H,H,H], [ZG39;F,H,F,H,H], [ZG40;F,H,H,F,H], [ZG41;F,H,H,H,F], [ZG42;H,F,F,H,H], [ZG43;H,F,H,F,H], [ZG44;H,F,F,F,H], [ZG45;F,F,H,F,F], [ZG46;F,F,F,F,F], [ZG47;Cl,Cl,H,H,H], [ZG48;Cl,H,Cl,H,H], [ZG49;Cl,H,H,Cl,H], [ZG50; Cl,H, H,H, Cl], [ZG51;H, Cl, Cl, H,H], [ZG52;H, Cl,H, Cl,H], [Z G53;CF₃,CF₃,H,H,H], [ZG54;CF₃,H,CF₃,H,H], [ZG55;CF₃,H,H,CF₃,H], [ZG56;CF₃,H,H,H,CF₃], [ZG57;H,CF₃,CF₃,H,H], [ZG58;Me,Me,H,H,H], [ZG59;Me,H,Me,H,H], [ZG60;Me,H,H,Me,H], [ZG61;Me,H,H,H,Me], [ZG62;H,Me,Me,H,H], [ZG63;OMe,OMe,H,H,H], [ZG64;OMe,H,OMe,H,H], [ZG65;OMe,H,H,OMe,H], [ZG66;OMe,H,H,H,OMe], [ZG67;H,OMe,OMe, H,H], [ZG68;H,OMe,H,OMe,H], [ZG69;F,Cl,H,H,H], [ZG70;Cl,F,H,H, H], [ZG71;F,H,Cl,H,H], [ZG72;Cl,H,F,H,H], [ZG73;F,H,H,Cl,H], [Z G74;Cl,H,H,F,H], [ZG75;F,H,H,H,Cl], [ZG76;H,F,Cl,H,H], [ZG77;H,Cl,F,H,H], [ZG78;H,F,H,Cl,H], [ZG79;F,Me,H,H,H], [ZG80;Me,F,H,H,H], [ZG81;F,H,Me,H,H], [ZG82;Me,H,F,H,H], [ZG83;F,H,H,Me,H], [ZG84;Me,H,H,F,H], [ZG85;F,H,H,H,Me], [ZG86;H,F,Me,H,H], [ZG8 7;H,Me,F,H,H], [ZG88;H,F,H,Me,H], [ZG89;F,OMe,H,H,H], [ZG90;OM e,F,H,H,H], [ZG91;F,H,OMe,H,H], [ZG92;OMe,H,F,H,H], [ZG93;F,H, H,OMe,H], [ZG94;OMe,H,H,F,H], [ZG95;F,H,H,H,OMe], [ZG96;H,F,OM e,H,H], [ZG97;H,OMe,F,H,H], [ZG98;H,F,H,OMe,H], [ZG99;F,CF₃,H, H,H], [ZG100;CF₃,F,H,H,H], [ZG101;F,H,CF₃,H,H], [ZG102;CF₃,H,F, H,H], [ZG103;F,H,H,CF₃,H], [ZG104;CF₃,H,H,F,H], [ZG105;F,H,H,H,CF₃], [ZG106;H,F,CF₃,H,H], [ZG107;H,CF₃,F,H,H], [ZG108;H,F,H,C F₃,H], [ZG109;Cl,OMe,H,H,H], [ZG110;OMe,Cl,H,H,H], [ZG111;Cl,H,OMe,H,H], [ZG112;OMe,H,Cl,H,H], [ZG113;Cl,H,H,OMe,H], [ZG114; OMe,H,H,Cl,H], [ZG115;Cl,H,H,H,OMe], [ZG116;H,Cl,OMe,H,H], [ZG 117;H,OMe,Cl,H,H], [ZG118;H,Cl,H,OMe,H], [ZG119;Cl,Me,H,H,H], [ZG120;Me,Cl,H,H,H], [ZG121;Cl,H,Me,H,H], [ZG122;Me,H,Cl,H,H], [ZG123;Cl,H,H,Me,H], [ZG124;Me,H,H,Cl,H], [ZG125;Cl,H,H,H,Me], [ZG126;H,Cl,Me,H,H], [ZG127;H,Me,Cl,H,H], [ZG128;H,Cl,H,Me, H], [ZG129;Cl,CF₃,H,H,H], [ZG130;CF₃,Cl,H,H,H], [ZG131;Cl,H,CF₃,H,H], [ZG132;CF₃,H,Cl,H,H], [ZG133;Cl,H,H,CF₃,H], [ZG134;CF₃,H,H,Cl,H], [ZG135;Cl,H,H,H,CF₃], [ZG136;H,Cl,CF₃,H,H], [ZG137;H,CF₃,Cl,H,H], [ZG138;H,Cl,H,CF₃,H], [ZG139;Me,CF₃,H,H,H], [ZG14 0;CF₃,Me,H,H,H], [ZG141;Me,H,CF₃,H,H], [ZG142;CF₃,H,Me,H,H], [Z G143;Me,H,H,CF₃,H], [ZG144;CF₃,H,H,Me,H], [ZG145;Me,H,H,H,CF₃][ZG146;H,Me,CF₃,H,H], [ZG147;H,CF₃,Me,H,H], [ZG148;H,Me,H,CF₃,H], [ZG149;Me,OMe,H,H,H], [ZG150;OMe,Me,H,H,H], [ZG151;Me,H,O Me,H,H], [ZG152;OMe,H,Me,H,H], [ZG153;Me,H,H,OMe,H], [ZG154;OM e,H,H,Me,H], [ZG155;Me,H,H,H,OMe], [ZG156;H,Me,OMe,H,H], [ZG15 7;H,OMe,Me,H,H], [ZG158;H,Me,H,OMe,H], [ZG159;OMe,CF₃,H,H,H], [ZG160;CF₃,OMe,H,H,H], [ZG161;OMe,H,CF₃,H,H], [ZG162;CF₃,H,OMe,H,H], [ZG163;OMe,H,H,CF₃,H], [ZG164;CF₃,H,H,OMe,H], [ZG165;OM e,H,H,H,CF₃], [ZG166;H,OMe,CF₃,H,H], [ZG167;H,CF₃,OMe,H,H], [ZG 168;H,OMe,H,CF₃,H], [ZG169;OPh,H,H,H,H], [ZG170; H,H,OPh,H,H]

A compound according to Compound (1E) wherein R¹ represents a chlorine atom, Q represents Q1, and a combination of R^{X2}, R^{X3}, R^{X4}, R^{X5} and R^{X6} represents any combination described in Combination G (hereinafter, referred to as Compound group SX1443).

A compound according to Compound (1E) wherein R¹ represents a methyl group, Q represents Q2, L¹ represents an oxygen atom, and a combination of R^{X2}, R^{X3}, R^{X4}, R^{X5} and R^{X6} represents any combination described in Combination G (hereinafter, referred to as Compound group SX1444).

A compound according to Compound (1E) wherein R¹ represents a chlorine atom, Q represents Q2, L¹ represents an oxygen atom, and a combination of R^{X2}, R^{X3}, R^{X4}, R^{X5} and R^{X6} represents any combination described in Combination G (hereinafter, referred to as Compound group SX1445).

A compound according to Compound (1E) wherein R¹ represents a methyl group, Q represents Q2, L¹ represents NH, and a combination of R^{X2}, R^{X3}, R^{X4}, R^{X5} and R^{X6} represents any combination described in Combination G (hereinafter, referred to as Compound group SX1446).

A compound according to Compound (1E) wherein R¹ represents a chlorine atom, Q represents Q2, L¹ represents NH, and a combination of R^{X2}, R^{X3}, R^{X4}, R^{X5} and R^{X6} represents any combination described in Combination G (hereinafter, referred to as Compound group SX1447).

A compound according to Compound (2G) wherein R¹ represents a methyl group, Q represents Q1, and a combination of structure of G² and substituents R^{X7}, R^{X8}, R^{X9} and R^{X10} applied to the structure of G² represents any combination described in Combination I (hereinafter, referred to as Compound group SX1448).

Combination I consists of substituent numbers ZI1 to ZI1327. Substituent numbers ZI1 to ZI1327 represent combinations of structure of G² and substituents of R^{X7}, R^{X8}, R^{X9} and R^{X10} applied to the structure of G² according to Compound (2G). And hereinafter, they are referred to as [substituent number; G², R^{X7}, R^{X8}, R^{X9}, R^{Xl}]. For example, the substituent number ZI2 refers to a combination wherein G² represents G1, R^{X7} represents an ethyl group, and each of R^{X8}, R^{X9} and R^{X10} represents a hydrogen atom.
Combination I: [ZI1;G1,H,H,H,H], [ZI2;G1,Et,H,H,H], [ZI3;G1,Pr,H,H,H], [ZI4;G 1,i-Pr,H,H,H], [ZI5;G1,c-Pr,H,H,H], [ZI6;G1,Ph,H,H,H], [ZI7;G1,OMe,H,H,H], [ZI8;G1,OEt, H,H,H], [ZI9;G1,OPh,H,H,H], [ZI10;G1,F,H,H,H], [ZI11;G1,C1,H,H,H], [ZI12;G1,Br,H,H,H], [ZI13;G1,I,H,H,H], [ZI14;G1,CN,H,H,H], [ZI15;G1,OCF₃,H,H,H], [ZI16;G1,OCHF₂,H,H,H], [ZI17;G1,CHF₂,H, H,H], [ZI18;G1,H,Et,H,H], [ZI19;G1,H,Pr,H,H], [ZI20;G1,H,i-Pr,H,H], [ZI21;G1,H,c-Pr,H,H], [ZI22;G1,H,Ph,H,H], [ZI23;G1,H,OMe,H,H], [ZI24;G1,H,O Et,H,H], [ZI25;G1,H,OPh,H,H], [ZI26;G1,H,F,H,H], [ZI27;G1,H,Cl,H,H], [ZI28;G1,H,Br,H,H], [ZI29;G1,H,I,H,H], [ZI30;G1,H,CN,H, H], [ZI31;G1,H,OCF₃,H,H], [ZI32;G1,H,OCHF₂,H,H], [ZI33;G1,H,CH F₂,H,H], [ZI34;G1,H,H,Et,H], [ZI35;G1,H,H,Pr,H], [ZI36;G1,H,H, i-Pr,H], [ZI37;G1,H,H,c-Pr,H], [ZI38;G1,H,H,Ph,H], [ZI39;G1,H,H,OMe,H], [ZI40;G1,H,H,O Et,H], [ZI41;G1,H,H,OPh,H], [ZI42;G1,H,H,F,H], [ZI43;G1,H,H,Cl,H], [ZI44;G1,H,H,Br,H], [ZI45;G1,H,H,I,H], [ZI46;G,H,H,CN,H], [ZI47;G1,H,H,OCF₃,H], [ZI48;G1,H,H,OCHF₂,H], [ZI49;G1,H,H,CH F₂,H], [ZI50;G1,H,H,H,Me], [ZI51;G1,H,H,H,Et], [ZI52;G1,H,H,H, Pr], [ZI53;G1,H,H,H,i-Pr], [ZI54;G1,H,H,H,c-Pr], [ZI55;G1,H,H,H,Ph], [ZI56;G1,H,H,H,OMe], [ZI57;G1,H,H,H,O Et], [ZI58;G1,H,H,H,OPh], [ZI59;G1,H,H,H,CF₃], [ZI60;G1,H,H,H, F], [ZI61;G1,H,H,H,Cl], [ZI62;G1,H,H,H,Br], [ZI63;G1,H,H,HI], [ZI64;G1,H,H,H,CN], [ZI65;G1,H,H,H,OCF₃], [ZI66;G1,H,H,H,OCHF₂], [ZI67;G1,H,H,H,CHF₂], [ZI68;G1,Me,Me,H,H], [ZI69;G1,Me,Et,H,H], [ZI70;G1,Me,OMe,H,H], [ZI71;G1,Me,OEt,H,H], [ZI72;G1,Me,S Me,H,H], [ZI73;G1,Me,F,H,H], [ZI74;G1,Me,Cl,H,H], [ZI75;G1,Me, CN,H,H], [ZI75;G1,Me,CF₃,H,H], [ZI76;G1,F,Me,H,H], [ZI77;G1,F, Et,H,H], [ZI78;G1,F,OMe,H,H], [ZI79;G1,F,OEt,H,H], [ZI80;G1,F, SMe,H,H], [ZI81;G1,F,F,H,H], [ZI82;G1,F,Cl,H,H], [ZI83;G1,F,CN,H,H], [ZI84;G1,F,CF₃,H,H], [ZI85;G1,Cl,Me,H,H], [ZI86;G1,Cl,E t,H,H], [ZI87;G1,Cl,OMe,H,H], [ZI88;G1,Cl,OEt,H,H], [ZI89;G1,Cl,SMe,H,H], [ZI90;G1,Cl,F,H,H], [ZI91;G1,Cl,Cl,H,H], [ZI92;G1, Cl,CN,H,H], [ZI93;G1ClCF₃,H,H], [ZI94;G1,OMe,Me,H,H], [ZI95;G1,OMe,Et,H,H], [ZI96;G1,OMe,OMe,H,H], [ZI97;G1,OMe,OEt,H,H], [Z I98;G1,OMe,SMe,H,H], [ZI99;G1,OMe,F,H,H], [ZI100;G1,OMe,Cl,H, H], [ZI101;G1,OMe,CN,H,H], [ZI102;G1OMeCF₃,H,H], [ZI103;G1,SMe,Me,H,H], [ZI104;G1,SMe,Et,H,H], [ZI105;G1,SMe,OMe,H,H], [ZI10 6;G1,SMe,OEt,H,H], [ZI107;G1,SMe,SMe,H,H], [ZI108;G1,SMe,F,H, H], [ZI109;G1,SMe,Cl,H,H], [ZI110;G1,SMe,CN,H,H], [ZI111;G1SMe CF₃,H,H], [ZI112;G1,CF₃,Me,H,H], [ZI113;G1,CF₃,Et,H,H], [ZI114; G1,CF₃,OMe,H,H], [ZI115;G1,CF₃,OEt,H,H], [ZI116;G1,CF₃,SMe,H,H], [ZI117;G1,CF₃,F,H,H], [ZI118;G1,CF₃,Cl,H,H], [ZI119;G1,CF₃,C N,H,H], [ZI120;G1,Me,H,Et,H], [ZI121;G1,Me,H,OMe,H], [ZI122;G1,Me,H,OEt,H], [ZI123;G1,Me,H,SMe,H], [ZI124;G1,Me,H,F,H], [ZI1 25;G1,Me,H,Cl,H], [ZI126;G1,Me,H,CN,H], [ZI127;G1,F,H,Me,H], [ZI128;G1,F,H,Et,H], [ZI129;G1,F,H,OMe,H], [ZI130;G1,F,H,OEt,H], [ZI131;G1,F,H,SMe,H], [ZI132;G1,F,H,F,H], [ZI133;G1,F,H,Cl, H], [ZI134;G1,F,H,CN,H], [ZI135;G1,F,H,CF₃,H], [ZI136;G1,Cl,H, Me,H], [ZI137;G1,Cl,H,Et,H], [ZI138;G1,Cl,H,OMe,H], [ZI139;G1, Cl,H,OEt,H], [ZI140;G1,Cl,H,SMe,H], [ZI141;G1,Cl,H,F,H], [ZI14 2;G1,Cl,H,Cl,H], [ZI143;G1,Cl,H,CN,H], [ZI144;G1ClH,CF₃,H], [Z I145;G1,OMe,H,Me,H], [ZI146;G1,OMe,H,Et,H], [ZI147;G1,OMe,H,O Me,H], [ZI148;G1,OMe,H,OEt,H], [ZI149;G1,OMe,H,SMe,H], [ZI150; G1,OMe,H,F,H], [ZI151;G1,OMe,H,Cl,H], [ZI152;G1,OMe,H,CN,H], [ZI153;GlOMeH,CF₃,H], [ZI154;G1,SMe,H,Me,H], [ZI155;G1,SMe,H,E t,H], [ZI156;G1,SMe,H,OMe,H], [ZI157;G1,SMe,H,OEt,H], [ZI158;G 1,SMe,H,SMe,H], [ZI159;G1,SMe,H,F,H], [ZI160;G1,SMe,H,Cl,H], [ZI61;G1,SMe,H,CN,H], [ZI162;G1SMeH,CF₃,H], [ZI163;G1,CF₃,H,M e,H], [ZI164;G1,CF₃,H,Et,H], [ZI165;G1,CF₃,H,OMe,H], [ZI166;G1,CF₃,H,OEt,H], [ZI167;G1,CF₃,H,SMe,H], [ZI168;G1,CF₃,H,F,H], [Z I169;G1,CF₃,H,Cl,H], [Z170;G1,CF₃,H,CN,H], [ZI171;G1,Me,H,H, Me], [ZI172;G1,Me,H,H,Et], [ZI173;G1,Me,H,H,OMe], [ZI174;G1,Me,H,H,OEt], [ZI175;G1,Me,H,H,SMe], [ZI176;G1,Me,H,H,F], [ZI177; G1,Me,H,H,Cl], [ZI178;G1,Me,H,H,CN], [ZI179;G1,Me,H,H,CF₃], [Z I180;G1,F,H,H,Me], [ZI181;G1,F,H,H,Et], [ZI182;G1,F,H,H,OMe], [ZI183;G1,F,H,H,OEt], [ZI184;G1,F,H,H,SMe], [ZI185;G1,F,H,H,F], [ZI186;G1,F,H,H,Cl], [ZI187;G1,F,H,H,CN], [ZI188;G1,F,H,H,C F₃], [ZI189;G1,Cl,H,H,Me], [ZI190;G1,Cl,H,H,Et], [ZI191;G1,Cl, H,H,OMe], [ZI192;G1,Cl,H,H,OEt], [ZI193;G1,Cl,H,H,SMe], [ZI194;G1,Cl,H,H,F], [ZI195;G1,Cl,H,H,Cl], [ZI196;G1,Cl,H,H,CN], [ZI 197;G1ClH,H,CF₃], [ZI198;G1,OMe,H,H,Me], [ZI199;G1,OMe,H,H,Et], [ZI200;G1,OMe,H,H,OMe], [ZI201;G1,OMe,H,H,OEt], [ZI202;G1,0 Me,H,H,SMe], [ZI203;G1,OMe,H,H,F], [ZI204;G1,OMe,H,H,Cl], [ZI2 05;G1,OMe,H,H,CN], [ZI206;GlOMeH,H,CF₃], [ZI207;G1,SMe,H,H,Me], [ZI208;G1,SMe,H,H,Et], [ZI209;G1,SMe,H,H,OMe], [ZI210;G1,SM e,H,H,OEt], [ZI211;G1,SMe,H,H,SMe], [ZI212;G1,SMe,H,H,F], [ZI2 13;G1,SMe,H,H,Cl], [ZI214;G1,SMe,H,H,CN], [ZI215;G1SMeH,H,CF₃], [ZI216;G1,CF₃,H,H,Me], [ZI217;G1,CF₃,H,H,Et], [ZI218;G1,CF₃, H,H,OMe], [ZI219;G1,CF₃,H,H,OEt], [ZI220;G1,CF₃,H,H,SMe], [ZI2 21;G1,CF₃,H,H,F], [ZI222;G1,CF₃,H,H,Cl], [ZI223;G1,CF₃,H,H,CN], [ZI224;G9,H,H,H,-], [ZI225;G9,Me,H,H,-], [ZI226;G9,Et,H,H,-], [ZI227;G9,Pr,H,H,-], [ZI228;G9,i-Pr,H,H,-], [ZI229;G9,c-Pr,H,H,-], [ZI230;G9,Ph,H,H,-], [ZI231;G9,OMe,H,H,-], [ZI232;G 9,OEt,H,H,-], [ZI233;G9,OPh,H,H,-], [ZI234;G9,F,H,H,-], [ZI235;G9,Br,H,H,-], [ZI236;G9,CN,H,H,-], [ZI237;G9,H,Et,H,-], [ZI23 8;G9,H,Pr,H,-], [ZI239;G9,H,i-Pr,H,-], [ZI240;G9,H,c-Pr,H,-], [ZI241;G9,H,Ph,H,-], [ZI242;G9,H,OMe,H,-], [ZI243;G9, H,OEt,H,-], [ZI244;G9,H,OPh,H,-], [ZI245;G9,H,CF₃,H,-], [ZI246;G9,H,F,H,-], [ZI247;G9,H,CN,H,-], [ZI248;G9,H,H,Et,-], [ZI249;G9,H,H,Pr,-], [ZI250;G9,H,H,i-Pr,-], [ZI251;G9,H,H,c-Pr,-], [ZI252;G9,H,H,Ph,-], [ZI253;G9,H,H,OMe,-], [ZI254;G9,H, H,OEt,-], [ZI255;G9,H,H,OPh,-], [ZI256;G9,H,H,CF₃,-], [ZI257;G 9,H,H,F,-], [ZI258;G9,H,H,CN,-], [ZI259;G10,H,H,H,-], [ZI260;G 10,Me,H,H,-], [ZI261;G10,Et,H,H,-], [ZI262;G10,Pr,H,H,-], [ZI2 63;G10,i-Pr,H,H,-], [ZI264;G10,c-Pr,H,H,-], [ZI265;G10,Ph,H,H,-], [ZI266;G10,OMe,H,H,-], [ZI267;G10,OEt,H,H, -], [ZI268;G10,OPh,H,H, -], [ZI269;G10,CF₃,H,H,-], [ZI270;G10,F,H,H,-], [ZI271;G10,Br,H,H,-], [ZI272;G10,CN,H,H,-], [ZI273;G10,H,Et,H,-], [ZI274;G10,H,Pr,H,-], [ZI275;G10,H, i-Pr,H,-], [ZI276;G10,H,c-Pr,H,-], [ZI277;G10,H,Ph,H,-], [ZI278;G10,H,OMe,H,-], [ZI279;G 10,H,OEt,H, -], [ZI280;G10,H,OPh,H, -], [ZI281;G10,H,CF₃,H,-], [ZI282;G10,H,F,H,-], [ZI283;G10,H,CN,H,-], [ZI284;G10,H,H,Et,-], [ZI285;G10,H,H,Pr,-], [ZI286;G10,H,H,i-Pr,-], [ZI287;G10,H,H,c-Pr,-], [ZI288;G10,H,H,Ph,-], [ZI289;G10,H,H,OMe,-], [ZI290;G10,H,H,OEt,-], [ZI291;G10,H,H,OPh,-], [ZI292;G10,H,H,CF₃,-], [ZI 293;G10,H,H,F,-], [ZI294;G10,H,H,CN,-], [ZI295;G22,H,H,-,-], [ZI296;G22,Me,H,-,-], [ZI297;G22,Et,H,-,-], [ZI298;G22,Pr,H,-,-], [ZI299;G22,i-Pr,H,-,-], [ZI300;G22,t-Bu,H,-,-], [ZI301;G22,c-Pr,H,-,-], [ZI302;G22,Ph,H,-,-], [ZI303;G22,OMe,H,-,-], [ZI304;G22,OEt,H,-,-], [ZI305;G22,OPh,H,-,-], [ZI306;G22,CHF₂,H,-,-], [ZI307;G22,F,H,-,-], [ZI308;G22,Br,H,-,-], [ZI309;G22,H,Me, -,-], [ZI310;G22,H,Et,-,-], [ZI311;G22,H,Pr,-,-], [ZI312;G22,H,i-Pr,-,-], [ZI313;G22,H,t-Bu,-,-], [ZI314;G22,H,c-Pr,-,-], [ZI315;G22,H,Ph,-,-], [ZI316;G22,H,OMe,-,-], [ZI317;G 22,H,OEt,-,-], [ZI318;G22,H,OPh,-,-], [ZI319;G22,H,OCF₃,-,-], [ZI320;G22,H,CN,-,-], [ZI321;G22,HCF₃,-,-], [ZI322;G22,HCHF₂, -,-], [ZI323;G22,H,F,-,-], [ZI324;G22,H,Cl,-,-], [ZI325;G22,H, Br,-,-], [ZI326;G22,H,I,-,-], [ZI327;G22,H,SMe,-,-]

A compound according to Compound (2G) wherein R¹ represents a chlorine atom, Q represents Q1, and a combination of structure of G² and substituents R^{X7}, R^{X8}, R^{X9} and R^{X10} applied to the structure of G² represents any combination described in Combination I (hereinafter, referred to as Compound group SX1449).

A compound according to Compound (2G) wherein R¹ represents a methyl group, Q represents Q2, L¹ represents an oxygen atom, and a combination of structure of G² and substituents R^{X7}, R^{X8}, R^{X9} and R^{X10} applied to the structure of G² represents any combination described in Combination I (hereinafter, referred to as Compound group SX1450).

A compound according to Compound (2G) wherein R¹ represents a chlorine atom, Q represents Q2, L¹ represents an oxygen atom, and a combination of structure of G² and substituents R^{X7}, R^{X8}, R^{X9} and R^{X10} applied to the structure of G² represents any combination described in Combination I (hereinafter, referred to as Compound group SX1451).

A compound according to Compound (2G) wherein R¹ represents a methyl group, Q represents Q2, L¹ represents NH, and a combination of structure of G² and substituents R^{X7}, R^{X8}, R^{X9} and R^{X10} applied to the structure of G² represents any combination described in Combination I (hereinafter, referred to as Compound group SX1452).

A compound according to Compound (2G) wherein R¹ represents a chlorine atom, Q represents Q2, L¹ represents NH, and a combination of structure of G² and substituents R^{X7}, R^{X8}, R^{X9} and R^{X10} applied to the structure of G² represents any combination described in Combination I (hereinafter, referred to as Compound group SX1453).

A compound represented by formula (2H) (hereinafter, referred to as Compound (2H)):

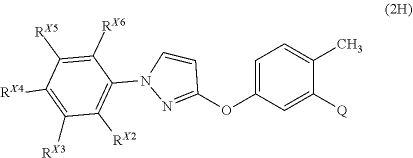

wherein Q represents Q1, and a combination of R^{X2}, R^{X3}, R^{X4}, R^{X5} and R^{X6} represents any combination described in Combination A (hereinafter, referred to as Compound group SX1454).

A compound according to Compound (2H) wherein Q represents Q2, L¹ represents an oxygen atom, and a combination of R^{X2}, R^{X3}, R^{X4}, R^{X5} and R^{X6} represents any combination described in Combination A (hereinafter, referred to as Compound group SX1455).

A compound according to Compound (2H) wherein Q represents Q2, L¹ represents NH, and a combination of R^{X2}, R^{X3}, R^{X4}, R^{X5} and R^{X6} represents any combination described in Combination A (hereinafter, referred to as Compound group SX1456).

A compound according to Compound (2H) wherein Q represents Q3, R³ represents a methoxy group, and a combination of R^{X2}, R^{X3}, R^{X4}, R^{X5} and R^{X6} represents any combination described in Combination A (hereinafter, referred to as Compound group SX1457).

A compound according to Compound (2H) wherein Q represents Q4, and a combination of R^{X2}, R^{X3}, R^{X4}, R^{X5} and R^{X6} represents any combination described in Combination A (hereinafter, referred to as Compound group SX1458).

A compound according to Compound (2H) wherein Q represents Q5, and a combination of R^{X2}, R^{X3}, R^{X4}, R^{X5} and R^{X6} represents any combination described in Combination A (hereinafter, referred to as Compound group SX1459).

A compound according to Compound (2H) wherein Q represents Q6, and a combination of R^{X2}, R^{X3}, R^{X4}, R^{X5} and R^{X6} represents any combination described in Combination A (hereinafter, referred to as Compound group SX1460).

A compound represented by formula (2I) (hereinafter, referred to as Compound (2I)):

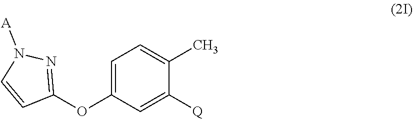

[wherein A represents any one of groups represented by formulae AA1 to AA4.]
wherein Q represents Q1, and a combination of structure of A and substituents R^{X11}, R^{X12}, R^{X13} and R^{X14} applied to the structure of A represents any combination described in Combination D (hereinafter, referred to as Compound group SX1461).

A compound according to Compound (2I) wherein Q represents Q2, L¹ represents an oxygen atom, and a combination of structure of A and substituents R^{X11}, R^{X12}, R^{X13} and R^{X14} applied to the structure of A represents any combination described in Combination D (hereinafter, referred to as Compound group SX1462).

A compound according to Compound (2I) wherein Q represents Q2, L¹ represents NH, and a combination of structure of A and substituents R^{X11}, R^{X12}, R^{X13} and R^{X14} applied to the structure of A represents any combination described in Combination D (hereinafter, referred to as Compound group SX1463).

A compound according to Compound (2I) wherein Q represents Q3, R³ represents a methoxy group, and a combination of structure of A and substituents R^{X11}, R^{X12}, R^{X13} and R^{X14} applied to the structure of A represents any combination described in Combination D (hereinafter, referred to as Compound group SX1464).

A compound according to Compound (2I) wherein Q represents Q4, and a combination of structure of A and substituents R^{X11}, R^{X12}, R^{X13} and R^{X14} applied to the structure of A represents any combination described in Combination D (hereinafter, referred to as Compound group SX1465).

A compound according to Compound (2I) wherein Q represents Q5, and a combination of structure of A and substituents R^{X11}, R^{X12}, R^{X13} and R^{X14} applied to the structure of A represents any combination described in Combination D (hereinafter, referred to as Compound group SX1466).

A compound according to Compound (2I) wherein Q represents Q6, and a combination of structure of A and substituents R^{X11}, R^{X12}, R^{X13} and R^{X14} applied to the structure of A represents any combination described in Combination D (hereinafter, referred to as Compound group SX1467).

A compound represented by formula (2J) (hereinafter, referred to as Compound (2J)):

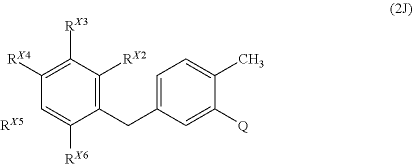

wherein Q represents Q1, and a combination of R^{X2}, R^{X3}, R^{X4}, R^{X5} and R^{X6} represents any combination described in Combination A (hereinafter, referred to as Compound group SX1468)

A compound according to Compound (2J) wherein Q represents Q2, L¹ represents an oxygen atom, and a combination of R^{X2}, R^{X3}, R^{X4}, R^{X5} and R^{X6} represents any combination described in Combination A (hereinafter, referred to as Compound group SX1469).

A compound according to Compound (2J) wherein Q represents Q2, L¹ represents NH, and a combination of R^{X2}, R^{X3}, R^{X4}, R^{X5} and R^{X6} represents any combination described in Combination A (hereinafter, referred to as Compound group SX1470).

A compound according to Compound (2J) wherein Q represents Q3, R³ represents a methoxy group, and a combination of R^{X2}, R^{X3}, R^{X4}, R^{X5} and R^{X6} represents any combination described in Combination A (hereinafter, referred to as Compound group SX1471).

A compound according to Compound (2J) wherein Q represents Q4, and a combination of R^{X2}, R^{X3}, R^{X4}, R^{X5} and R^{X6} represents any combination described in Combination A (hereinafter, referred to as Compound group SX1472).

A compound according to Compound (2J) wherein Q represents Q5, and a combination of R^{X2}, R^{X3}, R^{X4}, R^{X5} and R^{X6} represents any combination described in Combination A (hereinafter, referred to as Compound group SX1473).

A compound according to Compound (2J) wherein Q represents Q6, and a combination of R^{X2}, R^{X3}, R^{X4}, R^{X5} and R^{X6} represents any combination described in Combination A (hereinafter, referred to as Compound group SX1474).

A compound represented by formula (2K) (hereinafter, referred to as Compound (2K)):

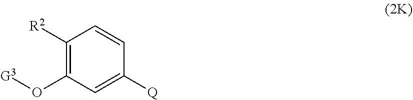

[wherein G³ represents any one of formulae G1 to G34.]
wherein R² represents a methyl group, Q represents Q1, and a combination of structure of G³ and substituents R^{X7}, R^{X8}, R^{X9} and R^{X10} applied to the structure of G³ represents any combination described in Combination B (hereinafter, referred to as Compound group SX1475).

A compound according to Compound (2K) wherein R² represents a chlorine atom, Q represents Q1, and a combination of structure of G³ and substituents R^{X7}, R^{X8}, R^{X9} and R^{X10} applied to the structure of G³ represents any combination described in Combination B (hereinafter, referred to as Compound group SX1476).

A compound according to Compound (2K) wherein R² represents an ethyl group, Q represents Q1, and a combination of structure of G³ and substituents R^{X7}, R^{X8}, R^{X9} and R^{X10} applied to the structure of G³ represents any combination described in Combination B (hereinafter, referred to as Compound group SX1477).

A compound according to Compound (2K) wherein R² represents a methoxy group, Q represents Q1, and a combination of structure of G³ and substituents R^{X7}, R^{X8}, R^{X9} and R^{X10} applied to the structure of G³ represents any combination described in Combination B (hereinafter, referred to as Compound group SX1478).

A compound according to Compound (2K) wherein R² represents a fluorine atom, Q represents Q1, and a combination of structure of G³ and substituents R^{X7}, R^{X8}, R^{X9} and R^{X10} applied to the structure of G³ represents any combination described in Combination B (hereinafter, referred to as Compound group SX1479).

A compound according to Compound (2K) wherein R² represents a cyclopropyl group, Q represents Q1, and a combination of structure of G³ and substituents R^{X7}, R^{X8}, R^{X9} and R^{X10} applied to the structure of G³ represents any combination described in Combination B (hereinafter, referred to as Compound group SX1480).

A compound according to Compound (2K) wherein R² represents a methyl group, Q represents Q2, L¹ represents an oxygen atom, and a combination of structure of G³ and substituents R^{X7}, R^{X8}, R^{X9} and R^{X10} applied to the structure of G³ represents any combination described in Combination B (hereinafter, referred to as Compound group SX1481).

A compound according to Compound (2K) wherein R² represents a chlorine atom, Q represents Q2, L¹ represents an oxygen atom, and a combination of structure of G³ and substituents R^{X7}, R^{X8}, R^{X9} and R^{X10} applied to the structure of G³ represents any combination described in Combination B (hereinafter, referred to as Compound group SX1482).

A compound according to Compound (2K) wherein R² represents a methyl group, Q represents Q2, L¹ represents NH, and a combination of structure of G³ and substituents R^{X7}, R^{X8}, R^{X9} and R^{X10} applied to the structure of G³ represents any combination described in Combination B (hereinafter, referred to as Compound group SX1483).

A compound according to Compound (2K) wherein R² represents a chlorine atom, Q represents Q2, L¹ represents NH, and a combination of structure of G³ and substituents R^{X7}, R^{X8}, R^{X9} and R^{X10} applied to the structure of G³ represents any combination described in Combination B (hereinafter, referred to as Compound group SX1484).

A compound according to Compound (2K) wherein R² represents a methyl group, Q represents Q3, R³ represents a methoxy group, and a combination of structure of G³ and substituents R^{X7}, R^{X8}, R^{X9} and R^{X10} applied to the structure of G³ represents any combination described in Combination B (hereinafter, referred to as Compound group SX1485).

A compound according to Compound (2K) wherein R² represents a chlorine atom, Q represents Q3, R³ represents a methoxy group, and a combination of structure of G³ and substituents R^{X7}, R^{X8}, R^{X9} and R^{X10} applied to the structure of G³ represents any combination described in Combination B (hereinafter, referred to as Compound group SX1486).

A compound according to Compound (2K) wherein R² represents a methyl group, Q represents Q4, and a combination of structure of G³ and substituents R^{X7}, R^{X8}, R^{X9} and R^{X10} applied to the structure of G³ represents any combination described in Combination B (hereinafter, referred to as Compound group SX1487).

A compound according to Compound (2K) wherein R² represents a chlorine atom, Q represents Q4, and a combination of structure of G³ and substituents R^{X7}, R^{X8}, R^{X9} and R^{X10} applied to the structure of G³ represents any combination described in Combination B (hereinafter, referred to as Compound group SX1488).

A compound according to Compound (2K) wherein R² represents a methyl group, Q represents Q5, and a combination of structure of G³ and substituents R^{X7}, R^{X8}, R^{X9} and R^{X10} applied to the structure of G³ represents any combination described in Combination B (hereinafter, referred to as Compound group SX1489).

A compound according to Compound (2K) wherein R² represents a chlorine atom, Q represents Q5, and a combination of structure of G³ and substituents R^{X7}, R^{X8}, R^{X9} and R^{X10} applied to the structure of G³ represents any combination described in Combination B (hereinafter, referred to as Compound group SX1490).

A compound according to Compound (2K) wherein R² represents a methyl group, Q represents Q6, and a combination of structure of G³ and substituents R^{X7}, R^{X8}, R^{X9} and R^{X10} applied to the structure of G³ represents any combination described in Combination B (hereinafter, referred to as Compound group SX1491).

A compound according to Compound (2K) wherein R² represents a chlorine atom, Q represents Q6, and a combination of structure of G³ and substituents R^{X7}, R^{X8}, R^{X9} and R^{X10} applied to the structure of G³ represents any combination described in Combination B (hereinafter, referred to as Compound group SX1492).

A compound represented by formula (2L) (hereinafter, referred to as Compound (2L)):

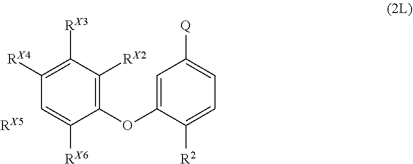

wherein R² represents a methyl group, Q represents Q1, and a combination of R^{X2}, R^{X3}, R^{X4}, R^{X5} and R^{X6} represents any combination described in Combination A (hereinafter, referred to as Compound group SX1493).

A compound according to Compound (2L) wherein R² represents a chlorine atom, Q represents Q1, and a combination of R^{X2}, R^{X3}, R^{X4}, R^{X5} and R^{X6} represents any combination described in Combination A (hereinafter, referred to as Compound group SX1494).

A compound according to Compound (2L) wherein R² represents a fluorine atom, Q represents Q1, and a combination of R^{X2}, R^{X3}, R^{X4}, R^{X5} and R^{X6} represents any combination described in Combination A (hereinafter, referred to as Compound group SX1495).

A compound according to Compound (2L) wherein R² represents an ethyl group, Q represents Q1, and a combination of R^{X2}, R^{X3}, R^{X4}, R^{X5} and R^{X6} represents any combination described in Combination A (hereinafter, referred to as Compound group SX1496).

A compound according to Compound (2L) wherein R² represents a methoxy group, Q represents Q1, and a combination of R^{X2}, R^{X3}, R^{X4}, R^{X5} and R^{X6} represents any combination described in Combination A (hereinafter, referred to as Compound group SX1497).

A compound according to Compound (2L) wherein R² represents a cyclopropyl group, Q represents Q1, and a combination of R^{X2}, R^{X3}, R^{X4}, R^{X5} and R^{X6} represents any combination described in Combination A (hereinafter, referred to as Compound group SX1498).

A compound according to Compound (2L) wherein R² represents a methyl group, Q represents Q2, L¹ represents an oxygen atom, and a combination of R^{X2}, R^{X3}, R^{X4}, R^{X5} and R^{X6} represents any combination described in Combination A (hereinafter, referred to as Compound group SX1499).

A compound according to Compound (2L) wherein R² represents a chlorine atom, Q represents Q2, L¹ represents an oxygen atom, and a combination of R^{X2}, R^{X3}, R^{X4}, R^{X5} and R^{X6} represents any combination described in Combination A (hereinafter, referred to as Compound group SX1500).

A compound according to Compound (2L) wherein R² represents a methyl group, Q represents Q2, L¹ represents NH, and a combination of R^{X2}, R^{X3}, R^{X4}, R^{X5} and R^{X6} represents any combination described in Combination A (hereinafter, referred to as Compound group SX1501).

A compound according to Compound (2L) wherein R² represents a chlorine atom, Q represents Q2, L¹ represents NH, and a combination of R^{X2}, R^{X3}, R^{X4}, R^{X5} and R^{X6} represents any combination described in Combination A (hereinafter, referred to as Compound group SX1502).

A compound according to Compound (2L) wherein R² represents a methyl group, Q represents Q3, R³ represents a methoxy group, and a combination of R^{X2}, R^{X3}, R^{X4}, R^{X5} and R^{X6} represents any combination described in Combination A (hereinafter, referred to as Compound group SX1503).

A compound according to Compound (2L) wherein R² represents a chlorine atom, Q represents Q3, R³ represents a methoxy group, and a combination of R^{X2}, R^{X3}, R^{X4}, R^{X5} and R^{X6} represents any combination described in Combination A (hereinafter, referred to as Compound group SX1504).

A compound according to Compound (2L) wherein R² represents a methyl group, Q represents Q4, and a combination of R^{X2}, R^{X3}, R^{X4}, R^{X5} and R^{X6} represents any combination described in Combination A (hereinafter, referred to as Compound group SX1505).

A compound according to Compound (2L) wherein R² represents a chlorine atom, Q represents Q4, and a combination of R^{X2}, R^{X3}, R^{X4}, R^{X5} and R^{X6} represents any combination described in Combination A (hereinafter, referred to as Compound group SX1506).

A compound according to Compound (2L) wherein R² represents a methyl group, Q represents Q5, and a combination of R^{X2}, R^{X3}, R^{X4}, R^{X5} and R^{X6} represents any combination described in Combination A (hereinafter, referred to as Compound group SX1507).

A compound according to Compound (2L) wherein R² represents a chlorine atom, Q represents Q5, and a combination of R^{X2}, R^{X3}, R^{X4}, R^{X5} and R^{X6} represents any combination described in Combination A (hereinafter, referred to as Compound group SX1508).

A compound according to Compound (2L) wherein R² represents a methyl group, Q represents Q6, and a combination of R^{X2}, R^{X3}, R^{X4}, R^{X5} and R^{X6} represents any combination described in Combination A (hereinafter, referred to as Compound group SX1509).

A compound according to Compound (2L) wherein R² represents a chlorine atom, Q represents Q6, and a combination of R^{X2}, R^{X3}, R^{X4}, R^{X5} and R^{X6} represents any combination described in Combination A (hereinafter, referred to as Compound group SX1510).

A compound represented by formula (2M) (hereinafter, referred to as Compound (2M)):

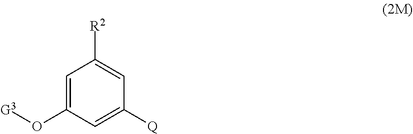

[wherein G³ represents any one of formulae G1 to G34.]
wherein R² represents a methyl group, Q represents Q1, and a combination of structure of G³ and substituents R^{X7}, R^{X8}, R^{X9} and R^{X10} applied to the structure of G³ represents any combination described in Combination B (hereinafter, referred to as Compound group SX1511).

A compound according to Compound (2M) wherein R² represents a chlorine atom, Q represents Q1, and a combination of structure of G³ and substituents R^{X7}, R^{X8}, R^{X9} and R^{X10} applied to the structure of G³ represents any combination described in Combination B (hereinafter, referred to as Compound group SX1512).

A compound according to Compound (2M) wherein R² represents an ethyl group, Q represents Q1, and a combination of structure of G³ and substituents R^{X7}, R^{X8}, R^{X9} and R^{X10} applied to the structure of G³ represents any combination described in Combination B (hereinafter, referred to as Compound group SX1513).

A compound according to Compound (2M) wherein R² represents a methoxy group, Q represents Q1, and a combination of structure of G³ and substituents R^{X7}, R^{X8}, R^{X9} and R^{X10} applied to the structure of G³ represents any combination described in Combination B (hereinafter, referred to as Compound group SX1514).

A compound according to Compound (2M) wherein R² represents a fluorine atom, Q represents Q1, and a combination of structure of G³ and substituents R^{X7}, R^{X8}, R^{X9} and R^{X10} applied to the structure of G³ represents any combination described in Combination B (hereinafter, referred to as Compound group SX1515).

A compound according to Compound (2M) wherein R² represents a cyclopropyl group, Q represents Q1, and a combination of structure of G³ and substituents R^{X7}, R^{X8}, R^{X9} and R^{X10} applied to the structure of G³ represents any combination described in Combination B (hereinafter, referred to as Compound group SX1516).

A compound according to Compound (2M) wherein R² represents a methyl group, Q represents Q2, L¹ represents an oxygen atom, and a combination of structure of G³ and substituents R^{X7}, R^{X8}, R^{X9} and R^{X10} applied to the structure of G³ represents any combination described in Combination B (hereinafter, referred to as Compound group SX1517).

A compound according to Compound (2M) wherein R² represents a chlorine atom, Q represents Q2, L¹ represents an oxygen atom, and a combination of structure of G³ and substituents R^{X7}, R^{X8}, R^{X9} and R^{X10} applied to the structure of G³ represents any combination described in Combination B (hereinafter, referred to as Compound group SX1518).

A compound according to Compound (2M) wherein R² represents a methyl group, Q represents Q2, L¹ represents NH, and a combination of structure of G³ and substituents R^{X7}, R^{X8}, R^{X9} and R^{X10} applied to the structure of G³ represents any combination described in Combination B (hereinafter, referred to as Compound group SX1519).

A compound according to Compound (2M) wherein R² represents a chlorine atom, Q represents Q2, L¹ represents NH, and a combination of structure of G³ and substituents R^{X7}, R^{X8}, R^{X9} and R^{X10} applied to the structure of G³ represents any combination described in Combination B (hereinafter, referred to as Compound group SX1520).

A compound according to Compound (2M) wherein R² represents a methyl group, Q represents Q3, R³ represents a methoxy group, and a combination of structure of G³ and substituents R^{X7}, R^{X8}, R^{X9} and R^{X10} applied to the structure of G³ represents any combination described in Combination B (hereinafter, referred to as Compound group SX1521).

A compound according to Compound (2M) wherein R² represents a chlorine atom, Q represents Q3, R³ represents a methoxy group, and a combination of structure of G³ and substituents R^{X7}, R^{X8}, R^{X9} and R^{X10} applied to the structure of G³ represents any combination described in Combination B (hereinafter, referred to as Compound group SX1522).

A compound according to Compound (2M) wherein R² represents a methyl group, Q represents Q4, and a combination of structure of G³ and substituents R^{X7}, R^{X8}, R^{X9} and R^{X10} applied to the structure of G³ represents any combination described in Combination B (hereinafter, referred to as Compound group SX1523).

A compound according to Compound (2M) wherein R² represents a chlorine atom, Q represents Q4, and a combination of structure of G³ and substituents R^{X7}, R^{X8}, R^{X9} and R^{X10} applied to the structure of G³ represents any combination described in Combination B (hereinafter, referred to as Compound group SX1524).

A compound according to Compound (2M) wherein R² represents a methyl group, Q represents Q5, and a combination of structure of G³ and substituents R^{X7}, R^{X8}, R^{X9} and R^{X10} applied to the structure of G³ represents any combination described in Combination B (hereinafter, referred to as Compound group SX1525).

A compound according to Compound (2M) wherein R² represents a chlorine atom, Q represents Q5, and a combination of structure of G³ and substituents R^{X7}, R^{X8}, R^{X9} and R^{X10} applied to the structure of G³ represents any combination described in Combination B (hereinafter, referred to as Compound group SX1526).

A compound according to Compound (2M) wherein R² represents a methyl group, Q represents Q6, and a combination of structure of G³ and substituents R^{X7}, R^{X8}, R^{X9} and R^{X10} applied to the structure of G³ represents any combination described in Combination B (hereinafter, referred to as Compound group SX1527).

A compound according to Compound (2M) wherein R² represents a chlorine atom, Q represents Q6, and a combination of structure of G³ and substituents R^{X7}, R^{X8}, R^{X9} and R^{X10} applied to the structure of G³ represents any combination described in Combination B (hereinafter, referred to as Compound group SX1528).

A compound represented by formula (2N) (hereinafter, referred to as Compound (2N)):

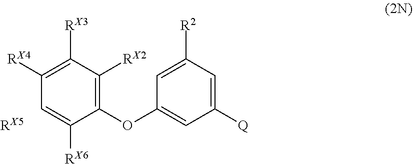

wherein R² represents a methyl group, Q represents Q1, and a combination of R^{X2}, R^{X3}, R^{X4}, R^{X5} and R^{X6} represents any combination described in Combination A (hereinafter, referred to as Compound group SX1529).

A compound according to Compound (2N) wherein R² represents a chlorine atom, Q represents Q1, and a combination of R^{X2}, R^{X3}, R^{X4}, R^{X5} and R^{X6} represents any combination described in Combination A (hereinafter, referred to as Compound group SX1530).

A compound according to Compound (2N) wherein R² represents a fluorine atom, Q represents Q1, and a combination of R^{X2}, R^{X3}, R^{X4}, R^{X5} and R^{X6} represents any combination described in Combination A (hereinafter, referred to as Compound group SX1531).

A compound according to Compound (2N) wherein R² represents an ethyl group, Q represents Q1, and a combination of R^{X2}, R^{X3}, R^{X4}, R^{X5} and R^{X6} represents any combination described in Combination A (hereinafter, referred to as Compound group SX1532).

A compound according to Compound (2N) wherein R² represents a methoxy group, Q represents Q1, and a combination of R^{X2}, R^{X3}, R^{X4}, R^{X5} and R^{X6} represents any combination described in Combination A (hereinafter, referred to as Compound group SX1533).

A compound according to Compound (2N) wherein R² represents a cyclopropyl group, Q represents Q1, and a combination of R^{X2}, R^{X3}, R^{X4}, R^{X5} and R^{X6} represents any combination described in Combination A (hereinafter, referred to as Compound group SX1534).

A compound according to Compound (2N) wherein R² represents a methyl group, Q represents Q2, L¹ represents an oxygen atom, and a combination of R^{X2}, R^{X3}, R^{X4}, R^{X5} and R^{X6} represents any combination described in Combination A (hereinafter, referred to as Compound group SX1535).

A compound according to Compound (2N) wherein R² represents a chlorine atom, Q represents Q2, L¹ represents an oxygen atom, and a combination of R^{X2}, R^{X3}, R^{X4}, R^{X5} and R^{X6} represents any combination described in Combination A (hereinafter, referred to as Compound group SX1536).

A compound according to Compound (2N) wherein R² represents a methyl group, Q represents Q2, L¹ represents NH, and a combination of R^{X2}, R^{X3}, R^{X4}, R^{X5} and R^{X6} represents any combination described in Combination A (hereinafter, referred to as Compound group SX1537).

A compound according to Compound (2N) wherein R² represents a chlorine atom, Q represents Q2, L¹ represents NH, and a combination of R^{X2}, R^{X3}, R^{X4}, R^{X5} and R^{X6} represents any combination described in Combination A (hereinafter, referred to as Compound group SX1538).

A compound according to Compound (2N) wherein R² represents a methyl group, Q represents Q3, R³ represents a methoxy group, and a combination of R^{X2}, R^{X3}, R^{X4}, R^{X5} and R^{X6} represents any combination described in Combination A (hereinafter, referred to as Compound group SX1539).

A compound according to Compound (2N) wherein R² represents a chlorine atom, Q represents Q3, R³ represents a methoxy group, and a combination of R^{X2}, R^{X3}, R^{X4}, R^{X5} and R^{X6} represents any combination described in Combination A (hereinafter, referred to as Compound group SX1540).

A compound according to Compound (2N) wherein R² represents a methyl group, Q represents Q4, and a combination of R^{X2}, R^{X3}, R^{X4}, R^{X5} and R^{X6} represents any combination described in Combination A (hereinafter, referred to as Compound group SX1541).

A compound according to Compound (2N) wherein R² represents a chlorine atom, Q represents Q4, and a combination of R^{X2}, R^{X3}, R^{X4}, R^{X5} and R^{X6} represents any combination described in Combination A (hereinafter, referred to as Compound group SX1542).

A compound according to Compound (2N) wherein R² represents a methyl group, Q represents Q5, and a combination of R^{X2}, R^{X3}, R^{X4}, R^{X5} and R^{X6} represents any combination described in Combination A (hereinafter, referred to as Compound group SX1543).

A compound according to Compound (2N) wherein R² represents a chlorine atom, Q represents Q5, and a combination of R^{X2}, R^{X3}, R^{X4}, R^{X5} and R^{X6} represents any combination described in Combination A (hereinafter, referred to as Compound group SX1544).

A compound according to Compound (2N) wherein R² represents a methyl group, Q represents Q6, and a combination of R^{X2}, R^{X3}, R^{X4}, R^{X5} and R^{X6} represents any combination described in Combination A (hereinafter, referred to as Compound group SX1545).

A compound according to Compound (2N) wherein R² represents a chlorine atom, Q represents Q6, and a combination of R^{X2}, R^{X3}, R^{X4}, R^{X5} and R^{X6} represents any combination described in Combination A (hereinafter, referred to as Compound group SX1546).

A compound represented by formula (20) (hereinafter, referred to as Compound (20)):

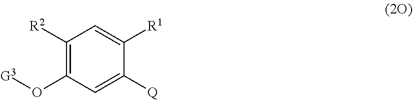

[wherein G³ represents any one of formulae G1 to G34.]
wherein R¹ represents a methyl group, R² represents a methyl group, Q represents Q1, and a combination of structure of G³ and substituents R^{X7}, R^{X8}, R^{X9} and R^{X10} applied to the structure of G³ represents any combination described in Combination B (hereinafter, referred to as Compound group SX1547).

A compound according to Compound (20) wherein R¹ represents a methyl group, R² represents a chlorine atom, Q represents Q1, and a combination of structure of G³ and substituents R^{X7}, R^{X8}, R^{X9} and R^{X10} applied to the structure of G³ represents any combination described in Combination B (hereinafter, referred to as Compound group SX1548).

A compound according to Compound (20) wherein R¹ represents a methyl group, R² represents an ethyl group, Q represents Q1, and a combination of structure of G³ and substituents R^{X7}, R^{X8}, R^{X9} and R^{X10} applied to the structure of G³ represents any combination described in Combination B (hereinafter, referred to as Compound group SX1549).

A compound according to Compound (20) wherein R¹ represents a methyl group, R² represents a methoxy group, Q represents Q1, and a combination of structure of G³ and substituents R^{X7}, R^{X8}, R^{X9} and R^{X10} applied to the structure of G³ represents any combination described in Combination B (hereinafter, referred to as Compound group SX1550).

A compound according to Compound (20) wherein R¹ represents a methyl group, R² represents a fluorine atom, Q represents Q1, and a combination of structure of G³ and substituents R^{X7}, R^{X8}, R^{X9} and R^{X10} applied to the structure of G³ represents any combination described in Combination B (hereinafter, referred to as Compound group SX1551).

A compound according to Compound (20) wherein R¹ represents a methyl group, R² represents a cyclopropyl group, Q represents Q1, and a combination of structure of G³ and substituents R^{X7}, R^{X8}, R^{X9} and R^{X10} applied to the structure of G³ represents any combination described in Combination B (hereinafter, referred to as Compound group SX1552).

A compound according to Compound (20) wherein R¹ represents a fluorine atom, R² represents a methyl group, Q represents Q1, and a combination of structure of G³ and substituents R^{X7}, R^{X8}, R^{X9} and R^{X10} applied to the structure of G³ represents any combination described in Combination B (hereinafter, referred to as Compound group SX1553).

A compound according to Compound (20) wherein R¹ represents a fluorine atom, R² represents a chlorine atom, Q represents Q1, and a combination of structure of G³ and substituents R^{X7}, R^{X8}, R^{X9} and R^{X10} applied to the structure of G³ represents any combination described in Combination B (hereinafter, referred to as Compound group SX1554).

A compound according to Compound (20) wherein R¹ represents a fluorine atom, R² represents an ethyl group, Q represents Q1, and a combination of structure of G³ and substituents R^{X7}, R^{X8}, R^{X9} and R^{X10} applied to the structure of G³ represents any combination described in Combination B (hereinafter, referred to as Compound group SX1555).

A compound according to Compound (20) wherein R¹ represents a fluorine atom, R² represents a methoxy group, Q represents Q1, and a combination of structure of G³ and substituents R^{X7}, R^{X8}, R^{X9} and R^{X10} applied to the structure of G³ represents any combination described in Combination B (hereinafter, referred to as Compound group SX1556).

A compound according to Compound (20) wherein R¹ represents a fluorine atom, R² represents a fluorine atom, Q represents Q1, and a combination of structure of G³ and substituents R^{X7}, R^{X8}, R^{X9} and R^{X10} applied to the structure of G³ represents any combination described in Combination B (hereinafter, referred to as Compound group SX1557).

A compound according to Compound (20) wherein R¹ represents a fluorine atom, R² represents a cyclopropyl group, Q represents Q1, and a combination of structure of G³ and substituents R^{X7}, R^{X8}, R^{X9} and R^{X10} applied to the structure of G³ represents any combination described in Combination B (hereinafter, referred to as Compound group SX1558).

A compound according to Compound (20) wherein R¹ represents a chlorine atom, R² represents a methyl group, Q represents Q1, and a combination of structure of G³ and substituents R^{X7}, R^{X8}, R^{X9} and R^{X10} applied to the structure of G³ represents any combination described in Combination B (hereinafter, referred to as Compound group SX1559).

A compound according to Compound (20) wherein R¹ represents a chlorine atom, R² represents a chlorine atom, Q represents Q1, and a combination of structure of G³ and substituents R^{X7}, R^{X8}, R^{X9} and R^{X10} applied to the structure of G³ represents any combination described in Combination B (hereinafter, referred to as Compound group SX1560).

A compound according to Compound (20) wherein R¹ represents a chlorine atom, R² represents an ethyl group, Q represents Q1, and a combination of structure of G³ and substituents R^{X7}, R^{X8}, R^{X9} and R^{X10} applied to the structure of G³ represents any combination described in Combination B (hereinafter, referred to as Compound group SX1561).

A compound according to Compound (20) wherein R¹ represents a chlorine atom, R² represents a methoxy group, Q represents Q1, and a combination of structure of G³ and substituents R^{X7}, R^{X8}, R^{X9} and R^{X10} applied to the structure of G³ represents any combination described in Combination B (hereinafter, referred to as Compound group SX1562).

A compound according to Compound (20) wherein R¹ represents a chlorine atom, R² represents a fluorine atom, Q represents Q1, and a combination of structure of G³ and substituents R^{X7}, R^{X8}, R^{X9} and R^{X10} applied to the structure of G³ represents any combination described in Combination B (hereinafter, referred to as Compound group SX1563).

A compound according to Compound (20) wherein R¹ represents a chlorine atom, R² represents a cyclopropyl group, Q represents Q1, and a combination of structure of G³ and substituents R^{X7}, R^{X8}, R^{X9} and R^{X10} applied to the structure of G³ represents any combination described in Combination B (hereinafter, referred to as Compound group SX1564).

A compound represented by formula (2P) (hereinafter, referred to as Compound (2P)):

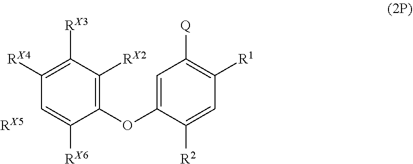

wherein R¹ represents a methyl group, R² represents a methyl group, Q represents Q1, and a combination of R^{X2}, R^{X3}, R^{X4}, R^{X5} and R^{X6} represents any combination described in Combination A (hereinafter, referred to as Compound group SX1565).

A compound according to Compound (2P) wherein R¹ represents a methyl group, R² represents a chlorine atom, Q represents Q1, and a combination of R^{X2}, R^{X3}, R^{X4}, R^{X5} and R^{X6} represents any combination described in Combination A (hereinafter, referred to as Compound group SX1566).

A compound according to Compound (2P) wherein R¹ represents a methyl group, R² represents a fluorine atom, Q represents Q1, and a combination of R^{X2}, R^{X3}, R^{X4}, R^{X5} and R^{X6} represents any combination described in Combination A (hereinafter, referred to as Compound group SX1567).

A compound according to Compound (2P) wherein R¹ represents a methyl group, R² represents an ethyl group, Q represents Q1, and a combination of R^{X2}, R^{X3}, R^{X4}, R^{X5} and R^{X6} represents any combination described in Combination A (hereinafter, referred to as Compound group SX1568).

A compound according to Compound (2P) wherein R¹ represents a methyl group, R² represents a methoxy group, Q represents Q1, and a combination of R^{X2}, R^{X3}, R^{X4}, R^{X5} and R^{X6} represents any combination described in Combination A (hereinafter, referred to as Compound group SX1569).

A compound according to Compound (2P) wherein R¹ represents a methyl group, R² represents a cyclopropyl group, Q represents Q1, and a combination of R^{X2}, R^{X3}, R^{X4}, R^{X5} and R^{X6} represents any combination described in Combination A (hereinafter, referred to as Compound group SX1570).

A compound according to Compound (2P) wherein R¹ represents a fluorine atom, R² represents a methyl group, Q represents Q1, and a combination of R^{X2}, R^{X3}, R^{X4}, R^{X5} and R^{X6} represents any combination described in Combination A (hereinafter, referred to as Compound group SX1571).

A compound according to Compound (2P) wherein R¹ represents a fluorine atom, R² represents a chlorine atom, Q represents Q1, and a combination of R^{X2}, R^{X3}, R^{X4}, R^{X5} and R^{X6} represents any combination described in Combination A (hereinafter, referred to as Compound group SX1572).

A compound according to Compound (2P) wherein R¹ represents a fluorine atom, R² represents a fluorine atom, Q represents Q1, and a combination of R^{X2}, R^{X3}, R^{X4}, R^{X5} and R^{X6} represents any combination described in Combination A (hereinafter, referred to as Compound group SX1573).

A compound according to Compound (2P) wherein R¹ represents a fluorine atom, R² represents an ethyl group, Q represents Q1, and a combination of R^{X2}, R^{X3}, R^{X4}, R^{X5} and R^{X6} represents any combination described in Combination A (hereinafter, referred to as Compound group SX1574).

A compound according to Compound (2P) wherein R¹ represents a fluorine atom, R² represents a methoxy group, Q represents Q1, and a combination of R^{X2}, R^{X3}, R^{X4}, R^{X5} and R^{X6} represents any combination described in Combination A (hereinafter, referred to as Compound group SX1575).

A compound according to Compound (2P) wherein R¹ represents a fluorine atom, R² represents a cyclopropyl group, Q represents Q1, and a combination of R^{X2}, R^{X3}, R^{X4}, R^{X5} and R^{X6} represents any combination described in Combination A (hereinafter, referred to as Compound group SX1576).

A compound according to Compound (2P) wherein R¹ represents a chlorine atom, R² represents a methyl group, Q represents Q1, and a combination of R^{X2}, R^{X3}, R^{X4}, R^{X5} and R^{X6} represents any combination described in Combination A (hereinafter, referred to as Compound group SX1577).

A compound according to Compound (2P) wherein R¹ represents a chlorine atom, R² represents a chlorine atom, Q represents Q1, and a combination of R^{X2}, R^{X3}, R^{X4}, R^{X5} and R^{X6} represents any combination described in Combination A (hereinafter, referred to as Compound group SX1578).

A compound according to Compound (2P) wherein R¹ represents a chlorine atom, R² represents a fluorine atom, Q represents Q1, and a combination of R^{X2}, R^{X3}, R^{X4}, R^{X5} and R^{X6} represents any combination described in Combination A (hereinafter, referred to as Compound group SX1579).

A compound according to Compound (2P) wherein R¹ represents a chlorine atom, R² represents an ethyl group, Q represents Q1, and a combination of R^{X2}, R^{X3}, R^{X4}, R^{X5} and R^{X6} represents any combination described in Combination A (hereinafter, referred to as Compound group SX1580).

A compound according to Compound (2P) wherein R¹ represents a chlorine atom, R² represents a methoxy group, Q represents Q1, and a combination of R^{X2}, R^{X3}, R^{X4}, R^{X5} and R^{X6} represents any combination described in Combination A (hereinafter, referred to as Compound group SX1581).

A compound according to Compound (2P) wherein R¹ represents a chlorine atom, R² represents a cyclopropyl group, Q represents Q1, and a combination of R^{X2}, R^{X3}, R^{X4}, R^{X5} and R^{X6} represents any combination described in Combination A (hereinafter, referred to as Compound group SX1582).

A compound represented by formula (3A) (hereinafter, referred to as Compound (3A)):

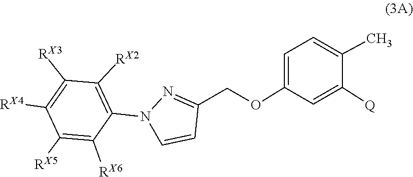

wherein Q represents Q1, and a combination of R^{X2}, R^{X3}, R^{X4}, R^{X5} and R^{X6} represents any combination described in Combination A (hereinafter, referred to as Compound group SX1583).

A compound according to Compound (3A) wherein Q represents Q2, L¹ represents an oxygen atom, and a combination of R^{X2}, R^{X3}, R^{X4}, R^{X5} and R^{X6} represents any combination described in Combination A (hereinafter, referred to as Compound group SX1584).

A compound according to Compound (3A) wherein Q represents Q2, L¹ represents NH, and a combination of R^{X2}, R^{X3}, R^{X4}, R^{X5} and R^{X6} represents any combination described in Combination A (hereinafter, referred to as Compound group SX1585).

A compound according to Compound (3A) wherein Q represents Q3, R³ represents a methoxy group, and a combination of R^{X2}, R^{X3}, R^{X4}, R^{X5} and R^{X6} represents any combination described in Combination A (hereinafter, referred to as Compound group SX1586).

A compound according to Compound (3A) wherein Q represents Q4, and a combination of R^{X2}, R^{X3}, R^{X4}, R^{X5} and R^{X6} represents any combination described in Combination A (hereinafter, referred to as Compound group SX1587).

A compound according to Compound (3A) wherein Q represents Q5, and a combination of R^{X2}, R^{X3}, R^{X4}, R^{X5} and R^{X6} represents any combination described in Combination A (hereinafter, referred to as Compound group SX1588).

A compound according to Compound (3A) wherein Q represents Q6, and a combination of R^{X2}, R^{X3}, R^{X4}, R^{X5} and R^{X6} represents any combination described in Combination A (hereinafter, referred to as Compound group SX1589).

A compound represented by formula (3B) (hereinafter, referred to as Compound (3B)):

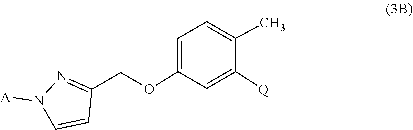

[wherein A represents any one of groups represented by formulae AA1 to AA4.]
wherein Q represents Q1, and a combination of structure of A and substituents R^{X11}, R^{X12}, R^{X13} and R^{X14} applied to the structure of A represents any combination described in Combination D (hereinafter, referred to as Compound group SX1590).

A compound according to Compound (3B) wherein Q represents Q2, L¹ represents an oxygen atom, and a combination of structure of A and substituents R^{X11}, R^{X12}, R^{X13} and R^{X14} applied to the structure of A represents any combination described in Combination D (hereinafter, referred to as Compound group SX1591).

A compound according to Compound (3B) wherein Q represents Q2, L¹ represents NH, and a combination of structure of A and substituents R^{X11}, R^{X12}, R^{X13} and R^{X14} applied to the structure of A represents any combination described in Combination D (hereinafter, referred to as Compound group SX1592).

A compound according to Compound (3B) wherein Q represents Q3, R³ represents a methoxy group, and a combination of structure of A and substituents R^{X11}, R^{X12}, R^{X13} and R^{X14} applied to the structure of A represents any combination described in Combination D (hereinafter, referred to as Compound group SX1593).

A compound according to Compound (3B) wherein Q represents Q4, and a combination of structure of A and substituents R^{X11}, R^{X12}, R^{X13} and R^{X14} applied to the structure of A represents any combination described in Combination D (hereinafter, referred to as Compound group SX1594).

A compound according to Compound (3B) wherein Q represents Q5, and a combination of structure of A and substituents R^{X11}, R^{X12}, R^{X13} and R^{X14} applied to the structure of A represents any combination described in Combination D (hereinafter, referred to as Compound group SX1595).

A compound according to Compound (3B) wherein Q represents Q6, and a combination of structure of A and substituents R^{X11}, R^{X12}, R^{X13} and R^{X14} applied to the structure of A represents any combination described in Combination D (hereinafter, referred to as Compound group SX1596).

A compound represented by formula (3C) (hereinafter, referred to as Compound (3C)):

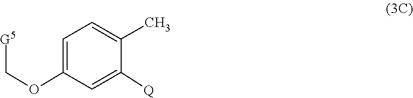

[wherein G⁵ represents any one of formulae G1 to G35.]
wherein Q represents Q1, and a combination of structure of G⁵ and substituents R^{X7}, R^{X8}, R^{X9} and R^{X10} applied to the structure of G⁵ represents any combination described in Combination B (hereinafter, referred to as Compound group SX1597).

A compound according to Compound (3C) wherein Q represents Q2, L¹ represents an oxygen atom, and a combination of structure of G⁵ and substituents R^{X7}, R^{X8}, R^{X9} and R^{X10} applied to the structure of G⁵ represents any combination described in Combination B (hereinafter, referred to as Compound group SX1598).

A compound according to Compound (3C) wherein Q represents Q2, L¹ represents NH, and a combination of structure of G⁵ and substituents R^{X7}, R^{X8}, R^{X9} and R^{X10} applied to the structure of G⁵ represents any combination described in Combination B (hereinafter, referred to as Compound group SX1599).

A compound according to Compound (3C) wherein Q represents Q3, R³ represents a methoxy group, and a combination of structure of G⁵ and substituents R^{X7}, R^{X8}, R^{X9} and R^{X10} applied to the structure of G⁵ represents any combination described in Combination B (hereinafter, referred to as Compound group SX1600).

A compound according to Compound (3C) wherein Q represents Q4, and a combination of structure of G⁵ and substituents R^{X7}, R^{X8}, R^{X9} and R^{X10} applied to the structure of G⁵ represents any combination described in Combination B (hereinafter, referred to as Compound group SX1601).

A compound according to Compound (3C) wherein Q represents Q5, and a combination of structure of G⁵ and substituents R^{X7}, R^{X8}, R^{X9} and R^{X10} applied to the structure of G⁵ represents any combination described in Combination B (hereinafter, referred to as Compound group SX1602).

A compound according to Compound (3C) wherein Q represents Q6, and a combination of structure of G⁵ and substituents R^{X7}, R^{X8}, R^{X9} and R^{X10} applied to the structure of G⁵ represents any combination described in Combination B (hereinafter, referred to as Compound group SX1603).

A compound represented by formula (3D) (hereinafter, referred to as Compound (3D)):

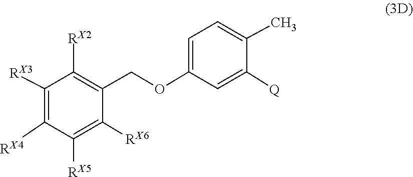

wherein Q represents Q1, and a combination of R^{X2}, R^{X3}, R^{X4}, R^{X5} and R^{X6} represents any combination described in Combination A (hereinafter, referred to as Compound group SX1604).

A compound according to Compound (3D) wherein Q represents Q2, L¹ represents an oxygen atom, and a combination of R^{X2}, R^{X3}, R^{X4}, R^{X5} and R^{X6} represents any combination described in Combination A (hereinafter, referred to as Compound group SX1605).

A compound according to Compound (3D) wherein Q represents Q2, L¹ represents NH, and a combination of R^{X2}, R^{X3}, R^{X4}, R^{X5} and R^{X6} represents any combination described in Combination A (hereinafter, referred to as Compound group SX1606).

A compound according to Compound (3D) wherein Q represents Q3, R³ represents a methoxy group, and a combination of R^{X2}, R^{X3}, R^{X4}, R^{X5} and R^{X6} represents any combination described in Combination A (hereinafter, referred to as Compound group SX1607).

A compound according to Compound (3D) wherein Q represents Q4, and a combination of R^{X2}, R^{X3}, R^{X4}, R^{X5} and R^{X6} represents any combination described in Combination A (hereinafter, referred to as Compound group SX1608).

A compound according to Compound (3D) wherein Q represents Q5, and a combination of R^{X2}, R^{X3}, R^{X4}, R^{X5} and R^{X6} represents any combination described in Combination A (hereinafter, referred to as Compound group SX1609).

A compound according to Compound (3D) wherein Q represents Q6, and a combination of R^{X2}, R^{X3}, R^{X4}, R^{X5} and R^{X6} represents any combination described in Combination A (hereinafter, referred to as Compound group SX1610).

A compound represented by formula (3E) (hereinafter, referred to as Compound (3E)):

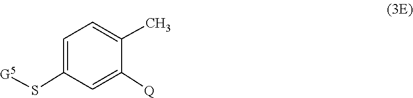

[wherein G⁵ represents any one of formulae G1 to G34.]
wherein Q represents Q1, and a combination of structure of G⁵ and substituents R^{X7}, R^{X8}, R^{X9} and R^{X10} applied to the structure of G⁵ represents any combination described in Combination B (hereinafter, referred to as Compound group SX1611).

A compound according to Compound (3E) wherein Q represents Q2, L¹ represents an oxygen atom, and a combination of structure of G⁵ and substituents R^{X7}, R^{X8}, R^{X9} and R^{X10} applied to the structure of G⁵ represents any combination described in Combination B (hereinafter, referred to as Compound group SX1612).

A compound according to Compound (3E) wherein Q represents Q2, L¹ represents NH, and a combination of structure of G⁵ and substituents R^{X7}, R^{X8}, R^{X9} and R^{X10} applied to the structure of G⁵ represents any combination described in Combination B (hereinafter, referred to as Compound group SX1613).

A compound according to Compound (3E) wherein Q represents Q3, R³ represents a methoxy group, and a combination of structure of G⁵ and substituents R^{X7}, R^{X8}, R^{X9} and R^{X10} applied to the structure of G⁵ represents any combination described in Combination B (hereinafter, referred to as Compound group SX1614).

A compound according to Compound (3E) wherein Q represents Q4, and a combination of structure of G⁵ and substituents R^{X7}, R^{X8}, R^{X9} and R^{X10} applied to the structure of G⁵ represents any combination described in Combination B (hereinafter, referred to as Compound group SX1615).

A compound according to Compound (3E) wherein Q represents Q5, and a combination of structure of G⁵ and substituents R^{X7}, R^{X8}, R^{X9} and R^{X10} applied to the structure of G⁵ represents any combination described in Combination B (hereinafter, referred to as Compound group SX1616).

A compound according to Compound (3E) wherein Q represents Q6, and a combination of structure of G⁵ and substituents R^{X7}, R^{X8}, R^{X9} and R^{X10} applied to the structure of G⁵ represents any combination described in Combination B (hereinafter, referred to as Compound group SX1617).

A compound represented by formula (3F) (hereinafter, referred to as Compound (3F)):

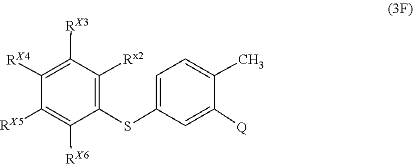

wherein Q represents Q1, and a combination of R^{X2}, R^{X3}, R^{X4}, R^{X5} and R^{X6} represents any combination described in Combination A (hereinafter, referred to as Compound group SX161).

A compound according to Compound (3F) wherein Q represents Q2, L¹ represents an oxygen atom, and a combination of R^{X2}, R^{X3}, R^{X4}, R^{X5} and R^{X6} represents any combination described in Combination A (hereinafter, referred to as Compound group SX1619).

A compound according to Compound (3F) wherein Q represents Q2, L¹ represents NH, and a combination of R^{X2}, R^{X3}, R^{X4}, R^{X5} and R^{X6} represents any combination described in Combination A (hereinafter, referred to as Compound group SX1620).

A compound according to Compound (3F) wherein Q represents Q3, R³ represents a methoxy group, and a combination of R^{X2}, R^{X3}, R^{X4}, R^{X5} and R^{X6} represents any combination described in Combination A (hereinafter, referred to as Compound group SX1621).

A compound according to Compound (3F) wherein Q represents Q4, and a combination of R^{X2}, R^{X3}, R^{X4}, R^{X5} and R^{X6} represents any combination described in Combination A (hereinafter, referred to as Compound group SX1622).

A compound according to Compound (3F) wherein Q represents Q5, and a combination of R^{X2}, R^{X3}, R^{X4}, R^{X5} and R^{X6} represents any combination described in Combination A (hereinafter, referred to as Compound group SX1623).

A compound according to Compound (3F) wherein Q represents Q6, and a combination of R^{X2}, R^{X3}, R^{X4}, R^{X5} and R^{X6} represents any combination described in Combination A (hereinafter, referred to as Compound group SX1624).

A compound represented by formula (3G) (hereinafter, referred to as Compound (3G)):

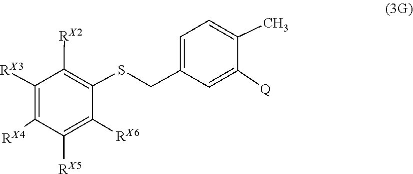

wherein Q represents Q1, and a combination of R^{X2}, R^{X3}, R^{X4}, R^{X5} and R^{X6} represents any combination described in Combination A (hereinafter, referred to as Compound group SX1625).

A compound according to Compound (3G) wherein Q represents Q2, L¹ represents an oxygen atom, and a combination of R^{X2}, R^{X3}, R^{X4}, R^{X5} and R^{X6} represents any combination described in Combination A (hereinafter, referred to as Compound group SX1626).

A compound according to Compound (3G) wherein Q represents Q2, L¹ represents NH, and a combination of R^{X2}, R^{X3}, R^{X4}, R^{X5} and R^{X6} represents any combination described in Combination A (hereinafter, referred to as Compound group SX1627).

A compound according to Compound (3G) wherein Q represents Q3, R³ represents a methoxy group, and a combination of R^{X2}, R^{X3}, R^{X4}, R^{X5} and R^{X6} represents any combination described in Combination A (hereinafter, referred to as Compound group SX1628).

A compound according to Compound (3G) wherein Q represents Q4, and a combination of R^{X2}, R^{X3}, R^{X4}, R^{X5} and R^{X6} represents any combination described in Combination A (hereinafter, referred to as Compound group SX1629).

A compound according to Compound (3G) wherein Q represents Q5, and a combination of R^{X2}, R^{X3}, R^{X4}, R^{X5} and R^{X6} represents any combination described in Combination A (hereinafter, referred to as Compound group SX1630).

A compound according to Compound (3G) wherein Q represents Q6, and a combination of R^{X2}, R^{X3}, R^{X4}, R^{X5} and R^{X6} represents any combination described in Combination A (hereinafter, referred to as Compound group SX1631).

A compound represented by formula (3H) (hereinafter, referred to as Compound (3H)):

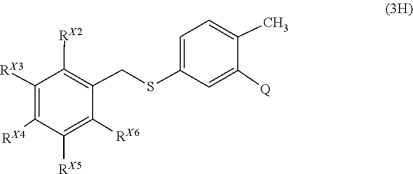

wherein Q represents Q1, and a combination of R^{X2}, R^{X3}, R^{X4}, R^{X5} and R^{X6} represents any combination described in Combination A (hereinafter, referred to as Compound group SX1632).

A compound according to Compound (3H) wherein Q represents Q2, L¹ represents an oxygen atom, and a combination of R^{X2}, R^{X3}, R^{X4}, R^{X5} and R^{X6} represents any combination described in Combination A (hereinafter, referred to as Compound group SX1633).

A compound according to Compound (3H) wherein Q represents Q2, L¹ represents NH, and a combination of R^{X2}, R^{X3}, R^{X4}, R^{X5} and R^{X6} represents any combination described in Combination A (hereinafter, referred to as Compound group SX1634).

A compound according to Compound (3H) wherein Q represents Q3, R³ represents a methoxy group, and a combination of R^{X2}, R^{X3}, R^{X4}, R^{X5} and R^{X6} represents any combination described in Combination A (hereinafter, referred to as Compound group SX1635).

A compound according to Compound (3H) wherein Q represents Q4, and a combination of R^{X2}, R^{X3}, R^{X4}, R^{X5} and R^{X6} represents any combination described in Combination A (hereinafter, referred to as Compound group SX1636).

A compound according to Compound (3H) wherein Q represents Q5, and a combination of R^{X2}, R^{X3}, R^{X4}, R^{X5} and R^{X6} represents any combination described in Combination A (hereinafter, referred to as Compound group SX1637).

A compound according to Compound (3H) wherein Q represents Q6, and a combination of R^{X2}, R^{X3}, R^{X4}, R^{X5} and R^{X6} represents any combination described in Combination A (hereinafter, referred to as Compound group SX1638).

A compound represented by formula (3I) (hereinafter, referred to as Compound (3I)):

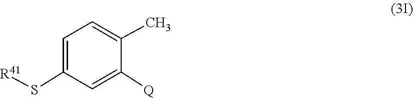

wherein Q represents Q1, and R⁴¹ represents any substituent selected from Group Y (hereinafter, referred to as Compound group SX1639).

A compound according to Compound (3I) wherein Q represents Q2, L¹ represents an oxygen atom, and R⁴¹ represents any substituent selected from Group Y (hereinafter, referred to as Compound group SX1640).

A compound according to Compound (3I) wherein Q represents Q2, L¹ represents NH, and R⁴¹ represents any substituent selected from Group Y (hereinafter, referred to as Compound group SX1641).

A compound according to Compound (3I) wherein Q represents Q3, R³ represents a methoxy group, and R⁴¹ represents any substituent selected from Group Y (hereinafter, referred to as Compound group SX1642).

A compound according to Compound (3I) wherein Q represents Q4, and R⁴¹ represents any substituent selected from Group Y (hereinafter, referred to as Compound group SX1643).

A compound according to Compound (3I) wherein Q represents Q5, and R⁴¹ represents any substituent selected from Group Y (hereinafter, referred to as Compound group SX1644).

A compound according to Compound (3I) wherein Q represents Q6, and R⁴¹ represents any substituent selected from Group Y (hereinafter, referred to as Compound group SX1645).

A compound according to Compound (3I) wherein Q represents Q1, and R⁴¹ represents any substituent selected from group Y2 (hereinafter, referred to as Compound group SX1646).

Group Y2: a group consisting of c-Pen, c-Hex, and 2-cyclopropylcyclopropyl.

A compound according to Compound (3I) wherein Q represents Q2, L¹ represents NH, and R⁴¹ represents any substituent selected from group Y2 (hereinafter, referred to as Compound group SX1647).

A compound according to Compound (3I) wherein Q represents Q2, L¹ represents an oxygen atom, and R⁴¹ represents any substituent selected from group Y2 (hereinafter, referred to as Compound group SX1648).

A compound according to Compound (3I) wherein Q represents Q3, R³ represents a methoxy group, and R⁴¹ represents any substituent selected from group Y2 (hereinafter, referred to as Compound group SX1649).

A compound according to Compound (3I) wherein Q represents Q4, and R⁴¹ represents any substituent selected from group Y2 (hereinafter, referred to as Compound group SX1650).

A compound according to Compound (3I) wherein Q represents Q5, and R⁴¹ represents any substituent selected from group Y2 (hereinafter, referred to as Compound group SX1651).

A compound according to Compound (3I) wherein Q represents Q6, and R⁴¹ represents any substituent selected from group Y2 (hereinafter, referred to as Compound group SX1652).

A compound according to Compound (3I) wherein Q represents Q7, L² represents an oxygen atom, and R⁴¹ represents any substituent selected from group Y2 (hereinafter, referred to as Compound group SX1653).

A compound according to Compound (3I) wherein Q represents Q7, L² represents NH, and R⁴¹ represents any substituent selected from group Y2 (hereinafter, referred to as Compound group SX1654).

A compound according to Compound (3I) wherein Q represents Q8A, and R⁴¹ represents any substituent selected from group Y2 (hereinafter, referred to as Compound group SX1655).

A compound represented by formula (3J) (hereinafter, referred to as Compound (3J)):

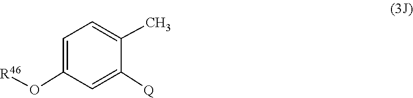

wherein Q represents Q1, and R⁴⁶ represents any substituent selected from group Y2 (hereinafter, referred to as Compound group SX1656).

A compound according to Compound (3J) wherein Q represents Q2, L¹ represents NH, and R⁴⁶ represents any substituent selected from group Y2 (hereinafter, referred to as Compound group SX1657).

A compound according to Compound (3J) wherein Q represents Q2, L¹ represents an oxygen atom, and R⁴⁶ represents any substituent selected from group Y2 (hereinafter, referred to as Compound group SX1658).

A compound according to Compound (3J) wherein Q represents Q3, R³ represents a methoxy group, and R⁴⁶ represents any substituent selected from group Y2 (hereinafter, referred to as Compound group SX1659).

A compound according to Compound (3J) wherein Q represents Q4, and R⁴⁶ represents any substituent selected from group Y2 (hereinafter, referred to as Compound group SX1660).

A compound according to Compound (3J) wherein Q represents Q5, and R⁴⁶ represents any substituent selected from group Y2 (hereinafter, referred to as Compound group SX1661).

A compound according to Compound (3J) wherein Q represents Q6, and R⁴⁶ represents any substituent selected from group Y2 (hereinafter, referred to as Compound group SX1662).

A compound according to Compound (3J) wherein Q represents Q7, L² represents an oxygen atom, and R⁴⁶ represents any substituent selected from group Y2 (hereinafter, referred to as Compound group SX1663).

A compound according to Compound (3J) wherein Q represents Q7, L² represents NH, and R⁴⁶ represents any substituent selected from group Y2 (hereinafter, referred to as Compound group SX1664).

A compound according to Compound (3J) wherein Q represents Q8A, and R⁴⁶ represents any substituent selected from group Y2 (hereinafter, referred to as Compound group SX1665).

A compound represented by formula (2Q) (hereinafter, referred to as Compound (2Q)):

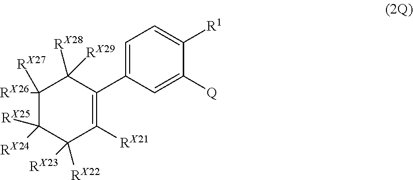

wherein Q represents Q3, R³ represents a methoxy group, R¹ represents a methyl group, and a combination of R^{X21}, R^{X22}, R^{X23}, R^{X24}, R^{X25}, R^{X26}, R^{X27}, R^{X28} and R^{X29} represents any combination described in Combination L (hereinafter, referred to as Compound group SX1666).

Combination L consists of substituent numbers ZL1 to ZL30. Substituent numbers ZL1 to ZL30 represent combinations of R^{X21}, R^{X22}, R^{X23}, R^{X24}, R^{X25}, R^{X26}, R^{X27}, R^{X28} and R^{X29} according to Compound (2Q). And hereinafter, they are referred to as [substituent number; R^{X21}, R^{X22}, R^{X23}, R^{X24}, R^{X25}, R^{X26}, R^{X27}, R^{X23}, R^{X29}]. For example, the substituent number ZL2 refers to a combination wherein R^{X21} represents a methyl group, and each of R^{X22}, R^{X23}, R^{X24}, R^{X25}, R^{X26}, R^{X27}, R^{X25} and R^{X29} represents a hydrogen atom.
Combination L: [ZL1;H,H,H,H,H,H,H,H,H], [ZL2;Me,H,H,H,H,H,H,H,H], [ZL3;F,H,H,H,H,H,H,H,H], [ZL4;Cl,H,H,H,H,H,H,H,H], [ZL5;H,F,F,H,H,H,H,H,H], [ZL6;Me,F,F,H,H,H,H,H,H], [ZL7;F,F,F,H,H,H,H,H,H], [ZL8;Cl,F,F,H,H,H,H,H,H], [ZL9;H,H,H,H,H,H,H,F,F], [ZL10;Me,H,H,H,H,H,H,F,F], [ZL11;F,H,H,H,H,H,H,F,F], [ZL12;Cl,H,H,H,H,H,H,F,F], [ZL13;H,Cl,Cl,H,H,H,H,H,H], [ZL14;Me,Cl,Cl,H,H,H,H,H,H], [ZL15;F,Cl,Cl,H,H,H,H,H,H], [ZL16;Cl,Cl,Cl,H,H,H,H,H,H], [ZL17; H,H,H,H,H,H,H,Cl,Cl], [ZL18;Me,H,H,H,H,H,H,Cl,Cl], [ZL19;F,H, H,H,H,H,H,Cl,Cl], [ZL20;Cl,H,H,H,H,H,H,Cl,Cl], [ZL21;H,H,H,F, F,H,H,H,H], [ZL22;H,H,H,Me,Me,H,H,H,H], [ZL23;H,H,H,Cl,Cl,H,H,H,H], [ZL24;H,H,H,i-Pr, H, H, H, H, H], [ZL25; H, H, H, CHF₂, H, H, H, H, H], [ZL26; H, H, H, CF₃, H,H,H,H,H], [ZL27;H,H,H,CN,H,H,H,H,H], [ZL28;H,H,H,OH,H,H,H,H, H], [ZL29;H,H,H,OMe,H,H,H,H,H], [ZL30;H,H,H,OCHF₂,H,H,H,H,H]

A compound according to Compound (2Q) wherein Q represents Q3, R³ represents a methoxy group, R¹ represents a chlorine atom, and a combination of R^{X21}, R^{X22}, R^{X23}, R^{X24}, R^{X25}, R^{X26}, R^{X27}, R^{X28} and R^{X29} represents any combination described in Combination L (hereinafter, referred to as Compound group SX1667).

A compound according to Compound (2Q) wherein Q represents Q4, R¹ represents a methyl group, and a combination of R^{X21}, R^{X22}, R^{X23}, R^{X24}, R^{X25}, R^{X26}, R^{X27}, R^{X28} and R^{X29} represents any combination described in Combination L (hereinafter, referred to as Compound group SX1668).

A compound according to Compound (2Q) wherein Q represents Q4, R¹ represents a chlorine atom, and a combination of R^{X21}, R^{X22}, R^{X23}, R^{X24}, R^{X25}, R^{X26}, R^{X27}, R^{X28} and R^{X29} represents any combination described in Combination L (hereinafter, referred to as Compound group SX1669).

A compound according to Compound (2Q) wherein Q represents Q5, R¹ represents a methyl group, and a combination of R^{X21}, R^{X22}, R^{X23}, R^{X24}, R^{X25}, R^{X26}, R^{X27}, R^{X28} and R^{X29} represents any combination described in Combination L (hereinafter, referred to as Compound group SX1670).

A compound according to Compound (2Q) wherein Q represents Q5, R¹ represents a chlorine atom, and a combination of R^{X21}, R^{X22}, R^{X23}, R^{X24}, R^{X25}, R^{X26}, R^{X27}, R^{X28} and R^{X29} represents any combination described in Combination L (hereinafter, referred to as Compound group SX1671).

A compound according to Compound (2Q) wherein Q represents Q6, R¹ represents a methyl group, and a combination of R^{X21}, R^{X22}, R^{X23}, R^{X24}, R^{X25}, R^{X26}, R^{X27}, R^{X28} and R^{X29} represents any combination described in Combination L (hereinafter, referred to as Compound group SX1672).

A compound according to Compound (2Q) wherein Q represents Q6, R¹ represents a chlorine atom, and a combination of R^{X2}1, R^{X22}, R^{X23}, R^{X24}, R^{X25}, R^{X26}, R^{X27}, R^{X28} and R^{X29} represents any combination described in Combination L (hereinafter, referred to as Compound group SX1673).

A compound according to Compound (2Q) wherein Q represents Q7, L² represents an oxygen atom, R¹ represents a methyl group, and a combination of R^{X21}, R^{X22}, R^{X23}, R^{X24}, R^{X25}, R^{X26}, R^{X27}, R^{X28} and R^{X29} represents any combination described in Combination L (hereinafter, referred to as Compound group SX1674).

A compound according to Compound (2Q) wherein Q represents Q7, L² represents an oxygen atom, R¹ represents a chlorine atom, and a combination of R^{X21}, R^{X22}, R^{X23}, R^{X24}, R^{X25}, R^{X26}, R^{X27}, R^{X28} and R^{X29} represents any combination described in Combination L (hereinafter, referred to as Compound group SX1675).

A compound according to Compound (2Q) wherein Q represents Q7, L² represents NH, R¹ represents a methyl group, and a combination of R^{X21}, R^{X22}, R^{X23}, R^{X24}, R^{X25}, R^{X26}, R^{X27}, R^{X28} and R^{X29} represents any combination described in Combination L (hereinafter, referred to as Compound group SX1676).

A compound according to Compound (2Q) wherein Q represents Q7, L² represents NH, R¹ represents a chlorine atom, and a combination of R^{X21}, R^{X22}, R^{X23}, R^{X24}, R^{X25}, R^{X26}, R^{X27}, R^{X28} and R^{X29} represents any combination described in Combination L (hereinafter, referred to as Compound group SX1677).

A compound according to Compound (2Q) wherein Q represents Q8A, R¹ represents a methyl group, and a combination of R^{X21}, R^{X22}, R^{X23}, R^{X24}, R^{X25}, R^{X26}, R^{X27}, R^{X28} and R^{X29} represents any combination described in Combination L (hereinafter, referred to as Compound group SX1678).

A compound according to Compound (2Q) wherein Q represents Q8A, R¹ represents a chlorine atom, and a combination of R^{X21}, R^{X22}, R^{X23}, R^{X24}, R^{X25}, R^{X26}, R^{X27}, R^{X28} and R^{X29} represents any combination described in Combination L (hereinafter, referred to as Compound group SX1679).

A compound represented by formula (2R) (hereinafter, referred to as Compound (2R)):

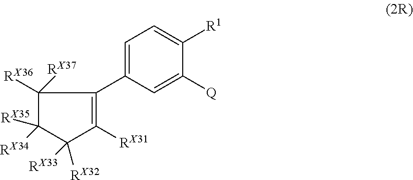

wherein Q represents Q3, R¹ represents a methyl group, R³ represents a methoxy group, and a combination of R^{X31}, R^{X32}, R^{X33}, R^{X34}, R^{X35}, R^{X36} and R^{X37} represents any combination described in Combination M (hereinafter, referred to as Compound group SX1680).

Combination M consists of substituent numbers ZM1 to ZM20. Substituent numbers ZM1 to ZM20 represent combinations of R^{X31}, R^{X32}, R^{X33}, R^{X34}, R^{X35}, R^{X36} and R^{X37} according to Compound (2R). And hereinafter, they are referred to as [substituent number; R^{X31}, R^{X32}, R^{X33}, R^{X34}, R^{X35}, R^{X36}, R^{X37}]. For example, the substituent number ZM2 refers to a combination wherein R^{X31} represents a methyl group, and each of R^{X32}, R^{X33}, R^{X34}, R^{X35}, R^{X36} and R^{X37} represents a hydrogen atom.
Combination M: [ZM1;H,H,H,H,H,H,H], [ZM2;Me,H,H,H,H,H,H], [ZM3;F,H,H,H,H,H,H], [ZM4;Cl,H,H,H,H,H,H], [ZM5;H,F,F,H,H,H,H], [ZM6;Me,F,F,H,H, H,H], [ZM7;F,F,F,H,H,H,H], [ZM8;Cl,F,F,H,H,H,H], [ZM9;H,H,H,H, H,F,F], [ZM10;Me,H,H,H,H,F,F], [ZM11;F,H,H,H,H,F,F], [ZM12;Cl, H,H,H,H,F,F], [ZM13;H,Cl,Cl,H,H,H,H], [ZM14;Me,Cl,Cl,H,H,H,H], [ZM15;F,Cl,Cl,H,H,H,H], [ZM16;Cl,Cl,Cl,H,H,H,H], [ZM17;H,H,H,H,H,Cl,Cl], [ZM18;Me,H,H,H,H,Cl,Cl], [ZM19;F,H,H,H,H,Cl,Cl], [ZM20;Cl,H,H,H,H,Cl,Cl]

A compound according to Compound (2R) wherein Q represents Q3, R¹ represents a chlorine atom, R³ represents a methoxy group, and a combination of R^{X31}, R^{X32}, P^{X33}, R^{X34}, R^{X35}, R^{X36} and R^{X37} represents any combination described in Combination M (hereinafter, referred to as Compound group SX1681).

A compound according to Compound (2R) wherein Q represents Q4, R¹ represents a methyl group, and a combination of R^{X31}, R^{X32}, R^{X33}, R^{X34}, R^{X35}, R^{X36} and R^{X37} represents any combination described in Combination M (hereinafter, referred to as Compound group SX1682).

A compound according to Compound (2R) wherein Q represents Q4, R¹ represents a chlorine atom, and a combination of R^{X31}, R^{X32}, R^{X33}, R^{X34}, R^{X35}, R^{X36} and R^{X37} represents any combination described in Combination M (hereinafter, referred to as Compound group SX1683).

A compound according to Compound (2R) wherein Q represents Q5, R¹ represents a methyl group, and a combination of R^{X31}, R^{X32}, R^{X33}, R^{X34}, R^{X35}, R^{X36} and R^{X37} represents any combination described in Combination M (hereinafter, referred to as Compound group SX1684).

A compound according to Compound (2R) wherein Q represents Q5, R¹ represents a chlorine atom, and a combination of R^{X31}, R^{X32}, R^{X33}, R^{X34}, R^{X35}, R^{X36} and R^{X37} represents any combination described in Combination M (hereinafter, referred to as Compound group SX1685).

A compound according to Compound (2R) wherein Q represents Q6, R¹ represents a methyl group, and a combination of R^{X31}, R^{X32}, R^{X33}, R^{X34}, R^{X35}, R^{X36} and R^{X37} represents any combination described in Combination M (hereinafter, referred to as Compound group SX1686).

A compound according to Compound (2R) wherein Q represents Q6, R¹ represents a chlorine atom, and a combination of R^{X31}, R^{X32}, R^{X33}, R^{X34}, R^{X35}, R^{X36} and R^{X37} represents any combination described in Combination M (hereinafter, referred to as Compound group SX1687).

A compound according to Compound (2R) wherein Q represents Q7, L² represents an oxygen atom, R¹ represents a methyl group, and a combination of R^{X31}, R^{X32}, R^{X33}, R^{X34}, R^{X35}, R^{X36} and R^{X37} represents any combination described in Combination M (hereinafter, referred to as Compound group SX1688).

A compound according to Compound (2R) wherein Q represents Q7, L² represents an oxygen atom, R¹ represents a chlorine atom, and a combination of R^{X31}, R^{X32}, R^{X33}, R^{X34}, R^{X35}, R^{X36} and R^{X37} represents any combination described in Combination M (hereinafter, referred to as Compound group SX1689).

A compound according to Compound (2R) wherein Q represents Q7, L² represents NH, R¹ represents a methyl group, and a combination of R^{X31}, R^{X32}, R^{X33}, R^{X34}, R^{X35}, R^{X36} and R^{X37} represents any combination described in Combination M (hereinafter, referred to as Compound group SX1690).

A compound according to Compound (2R) wherein Q represents Q7, L² represents NH, R¹ represents a chlorine atom, and a combination of R^{X31}, R^{X32}, R^{X33}, R^{X34}, R^{X35}, R^{X36} and R^{X37} represents any combination described in Combination M (hereinafter, referred to as Compound group SX1691).

A compound according to Compound (2R) wherein Q represents Q8A, R¹ represents a methyl group, and a combination of R^{X31}, R^{X32}, R^{X33}, R^{X34}, R^{X35}, R^{X36} and R^{X37} represents any combination described in Combination M (hereinafter, referred to as Compound group SX1692).

A compound according to Compound (2R) wherein Q represents Q8A, R¹ represents a chlorine atom, and a combination of R^{X31}, R^{X32}, R^{X33}, R^{X34}, R^{X35}, R^{X36} and R^{X37} represents any combination described in Combination M (hereinafter, referred to as Compound group SX1693).

A compound represented by formula (2S) (hereinafter, referred to as Compound (2S)):

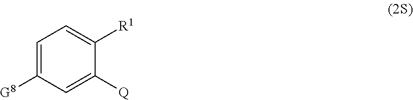

[wherein G⁸ represents any one of formulae G36 to G42

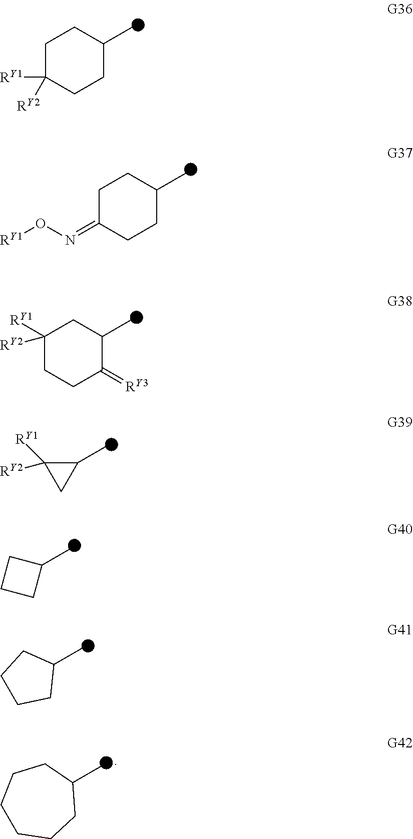

wherein Q represents Q1, R¹ represents a methyl group, and a combination of structure of G⁸ and substituents R^{Y1}, R^{Y2} and R^{Y3} applied to the structure of G⁸ represents any combination described in Combination N (hereinafter, referred to as Compound group SX1694).

Combination N consists of substituent numbers ZN1 to ZN26. Substituent numbers ZN1 to ZN26 represent combinations of structure of G⁸ and substituents of R^{Y1}, R^{Y2} and R^{Y3} applied to the structure of G⁸ according to compounds represented by Compound (2S). And hereinafter, they are referred to as [substituent number; G⁸, R^{Y1}, R^{Y2}, R^{Y3}]. For example, the substituent number ZN2 refers to a combination wherein G⁸ represents G36, and each of R^{Y1} and R^{Y2} represents a fluorine atom.
Combination N: [ZN1;G36,H,H,-], [ZN2;G36,F,F,-], [ZN3;G36,Me,Me,-], [ZN4;G36, CHF₂,H, -], [ZN5;G36, CF₃,H, -], [ZN6;G36, i-Pr,H,-], [ZN7;G36,CN,H, -], [ZN8;G36,OMe,H, -], [ZN9;G36,OCHF₂,H,-], [ZN10;G37,Me,-,-], [ZN11;G37,Et,-,-], [ZN12;G37,n-Pr,-,-], [ZN13;G38,Me,Me,NOMe], [ZN14;G38,Me,Me,O], [ZN15;G38, H,H,NOMe], [ZN16;G38,H,H,O], [ZN17;G39,H,H,-], [ZN18;G39,c-Pr,H,-], [ZN19;G39,Me,H,-], [ZN20;G39,Et,H,-], [ZN21;G39,n-Pr,H,-], [ZN22;G39,i-Pr,H,-], [ZN23;G39,Ph,H,-], [ZN24;G40,-,-,-], [ZN25;G41,-,-,-], [ZN26;G42,-,-,-]

A compound according to Compound (2S) wherein Q represents Q1, R¹ represents a chlorine atom, and a combination of structure of G⁸ and substituents R^{Y1}, R^{Y2} and R^{Y3} applied to the structure of G⁸ represents any combination described in Combination N (hereinafter, referred to as Compound group SX1695).

A compound according to Compound (2S) wherein Q represents Q2, L¹ represents an oxygen atom, R¹ represents a methyl group, and a combination of structure of G⁸ and substituents R^{Y1}, R^{Y2} and R^{Y3} applied to the structure of G⁸ represents any combination described in Combination N (hereinafter, referred to as Compound group SX1696).

A compound according to Compound (2S) wherein Q represents Q2, L¹ represents an oxygen atom, R¹ represents a chlorine atom, and a combination of structure of G⁸ and substituents R^{Y1}, R^{Y2} and R^{Y3} applied to the structure of G⁸ represents any combination described in Combination N (hereinafter, referred to as Compound group SX1697).

A compound according to Compound (2S) wherein Q represents Q2, L¹ represents NH, R¹ represents a methyl group, and a combination of structure of G⁸ and substituents R^{Y1}, R^{Y2} and R^{Y3} applied to the structure of G⁸ represents any combination described in Combination N (hereinafter, referred to as Compound group SX1698).

A compound according to Compound (2S) wherein Q represents Q2, L¹ represents NH, R¹ represents a chlorine atom, and a combination of structure of G⁸ and substituents R^{Y1}, R^{Y2} and R^{Y3} applied to the structure of G⁸ represents any combination described in Combination N (hereinafter, referred to as Compound group SX1699).

A compound according to Compound (2S) wherein Q represents Q3, R¹ represents a methyl group, R³ represents a methoxy group, and a combination of structure of G⁸ and substituents R^{Y1}, R^{Y2} and R^{Y3} applied to the structure of G⁸ represents any combination described in Combination N (hereinafter, referred to as Compound group SX1700).

A compound according to Compound (2S) wherein Q represents Q3, R¹ represents a chlorine atom, R³ represents a methoxy group, and a combination of structure of G⁸ and substituents R^{Y1}, R^{Y2} and R^{Y3} applied to the structure of G⁸ represents any combination described in Combination N (hereinafter, referred to as Compound group SX1701).

A compound according to Compound (2S) wherein Q represents Q4, R¹ represents a methyl group, and a combination of structure of G⁸ and substituents R^{Y1}, R^{Y2} and R^{Y3} applied to the structure of G⁸ represents any combination described in Combination N (hereinafter, referred to as Compound group SX1702).

A compound according to Compound (2S) wherein Q represents Q4, R¹ represents a chlorine atom, and a combination of structure of G⁸ and substituents R^{Y1}, R^{Y2} and R^{Y3} applied to the structure of G⁸ represents any combination described in Combination N (hereinafter, referred to as Compound group SX1703).

A compound according to Compound (2S) wherein Q represents Q5, R¹ represents a methyl group, and a combination of structure of G⁸ and substituents R^{Y1}, R^{Y2} and R^{Y3} applied to the structure of G⁸ represents any combination described in Combination N (hereinafter, referred to as Compound group SX1704).

A compound according to Compound (2S) wherein Q represents Q5, R¹ represents a chlorine atom, and a combination of structure of G⁸ and substituents R^{Y1}, R^{Y2} and R^{Y3} applied to the structure of G⁸ represents any combination described in Combination N (hereinafter, referred to as Compound group SX1705).

A compound according to Compound (2S) wherein Q represents Q6, R¹ represents a methyl group, and a combination of structure of G⁸ and substituents R^{Y1}, R^{Y2} and R^{Y3} applied to the structure of G⁸ represents any combination described in Combination N (hereinafter, referred to as Compound group SX1706).

A compound according to Compound (2S) wherein Q represents Q6, R¹ represents a chlorine atom, and a combination of structure of G⁸ and substituents R^{Y1}, R^{Y2} and R^{Y3} applied to the structure of G⁸ represents any combination described in Combination N (hereinafter, referred to as Compound group SX1707).

A compound according to Compound (2S) wherein Q represents Q7, L² represents an oxygen atom, R¹ represents a methyl group, and a combination of structure of G⁸ and substituents R^{Y1}, R^{Y2} and R^{Y3} applied to the structure of G⁸ represents any combination described in Combination N (hereinafter, referred to as Compound group SX1708).

A compound according to Compound (2S) wherein Q represents Q7, L² represents an oxygen atom, R¹ represents a chlorine atom, and a combination of structure of G⁸ and substituents R^{Y1}, R^{Y2} and R^{Y3} applied to the structure of G⁸ represents any combination described in Combination N (hereinafter, referred to as Compound group SX1709).

A compound according to Compound (2S) wherein Q represents Q7, L² represents NH, R¹ represents a methyl group, and a combination of structure of G⁸ and substituents R^{Y1}, R^{Y2} and R^{Y3} applied to the structure of G⁸ represents any combination described in Combination N (hereinafter, referred to as Compound group SX1710).

A compound according to Compound (2S) wherein Q represents Q7, L² represents NH, R¹ represents a chlorine atom, and a combination of structure of G⁸ and substituents R^{Y1}, R^{Y2} and R^{Y3} applied to the structure of G⁸ represents any combination described in Combination N (hereinafter, referred to as Compound group SX1711).

A compound according to Compound (2S) wherein Q represents Q8A, R¹ represents a methyl group, and a combination of structure of G⁸ and substituents R^{Y1}, R^{Y2} and R^{Y3} applied to the structure of G⁸ represents any combination described in Combination N (hereinafter, referred to as Compound group SX1712).

A compound according to Compound (2S) wherein Q represents Q8A, R¹ represents a chlorine atom, and a combination of structure of G⁸ and substituents R^{Y1}, R^{Y2} and R^{Y3} applied to the structure of G⁸ represents any combination described in Combination N (hereinafter, referred to as Compound group SX1713).

A compound represented by formula (2T) (hereinafter, referred to as Compound (2T)):

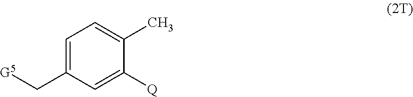

[wherein G⁵ represents any one of formulae G1 to G35.]
wherein Q represents Q1, and a combination of structure of G⁵ and substituents R^{X7}, R^{X8}, R^{X9} and R^{X10} applied to the structure of G⁵ represents any combination described in Combination B (hereinafter, referred to as Compound group SX1714).

A compound according to Compound (2T) wherein Q represents Q2, L¹ represents an oxygen atom, and a combination of structure of G⁵ and substituents R^{X7}, R^{X8}, R^{X9} and R^{X10} applied to the structure of G⁵ represents any combination described in Combination B (hereinafter, referred to as Compound group SX1715).

A compound according to Compound (2T) wherein Q represents Q2, L¹ represents NH, and a combination of structure of G⁵ and substituents R^{X7}, R^{X8}, R^{X9} and R^{X10} applied to the structure of G⁵ represents any combination described in Combination B (hereinafter, referred to as Compound group SX1716).

A compound according to Compound (2T) wherein Q represents Q3, R³ represents a methoxy group, and a combination of structure of G⁵ and substituents R^{X7}, R^{X8}, R^{X9} and R^{X10} applied to the structure of G⁵ represents any combination described in Combination B (hereinafter, referred to as Compound group SX1717).

A compound according to Compound (2T) wherein Q represents Q4, and a combination of structure of G⁵ and substituents R^{X7}, R^{X8}, R^{X9} and R^{X10} applied to the structure of G⁵ represents any combination described in Combination B (hereinafter, referred to as Compound group SX1718).

A compound according to Compound (2T) wherein Q represents Q5, and a combination of structure of G⁵ and substituents R^{X7}, R^{X8}, R^{X9} and R^{X10} applied to the structure of G⁵ represents any combination described in Combination B (hereinafter, referred to as Compound group SX1719).

A compound according to Compound (2T) wherein Q represents Q6, and a combination of structure of G⁵ and substituents R^{X7}, R^{X8}, R^{X9} and R^{X10} applied to the structure of G⁵ represents any combination described in Combination B (hereinafter, referred to as Compound group SX1720).

Next, specific examples of the Composition of the present invention are shown below. The present compound T refers to the present compound 4-1, 4-6, 4-9, 4-12, 4-13, 4-14, 4-15, 4-17, 4-36, 4-38, 4-40, 4-54, 4-55, 4-56, 4-57, 4-58, 4-74, 14-4, 14-6, 14-7, 14-8, 14-9, 14-10, 14-12, 14-14, 14-35, 14-36, 14-37, 14-38, 14-39, 14-40, 14-41, 14-42 and 14-43.
Composition group MX1: a composition for controlling plant diseases comprising any one of the present compound T and picoxystrobin at a ratio of 0.1:1;
Composition group MX2: a composition for controlling plant diseases comprising any one of the present compound T and picoxystrobin at a ratio of 1:1;
Composition group MX3: a composition for controlling plant diseases comprising any one of the present compound T and picoxystrobin at a ratio of 10:1;
Composition group MX4: a composition for controlling plant diseases comprising any one of the present compound T and pyraclostrobin at a ratio of 0.1:1;
Composition group MX5: a composition for controlling plant diseases comprising any one of the present compound T and pyraclostrobin at a ratio of 1:1;
Composition group MX6: a composition for controlling plant diseases comprising any one of the present compound T and pyraclostrobin at a ratio of 10:1;
Composition group MX7: a composition for controlling plant diseases comprising any one of the present compound T and metyltetraprole at a ratio of 0.1:1;
Composition group MX8: a composition for controlling plant diseases comprising any one of the present compound T and metyltetraprole at a ratio of 1:1;
Composition group MX9: a composition for controlling plant diseases comprising any one of the present compound T and metyltetraprole at a ratio of 10:1;
Composition group MX10: a composition for controlling plant diseases comprising any one of the present compound T and fenpicoxamid at a ratio of 0.1:1;
Composition group MX11: a composition for controlling plant diseases comprising any one of the present compound T and fenpicoxamid at a ratio of 1:1;
Composition group MX12: a composition for controlling plant diseases comprising any one of the present compound T and fenpicoxamid at a ratio of 10:1;
Composition group MX13: a composition for controlling plant diseases comprising any one of the present compound T and florylpicoxamid at a ratio of 0.1:1;
Composition group MX14: a composition for controlling plant diseases comprising any one of the present compound T and florylpicoxamid at a ratio of 1:1;
Composition group MX15: a composition for controlling plant diseases comprising any one of the present compound T and florylpicoxamid at a ratio of 10:1;
Composition group MX16: a composition for controlling plant diseases comprising any one of the present compound T and fluxapyroxad at a ratio of 0.1:1;
Composition group MX17: a composition for controlling plant diseases comprising any one of the present compound T and fluxapyroxad at a ratio of 1:1;
Composition group MX18: a composition for controlling plant diseases comprising any one of the present compound T and fluxapyroxad at a ratio of 10:1;
Composition group MX19: a composition for controlling plant diseases comprising any one of the present compound T and benzovindiflupyr at a ratio of 0.1:1;
Composition group MX20: a composition for controlling plant diseases comprising any one of the present compound T and benzovindiflupyr at a ratio of 1:1;
Composition group MX21: a composition for controlling plant diseases comprising any one of the present compound T and benzovindiflupyr at a ratio of 10:1;
Composition group MX22: a composition for controlling plant diseases comprising any one of the present compound T and fluindapyr at a ratio of 0.1:1;
Composition group MX23: a composition for controlling plant diseases comprising any one of the present compound T and fluindapyr at a ratio of 1:1;
Composition group MX24: a composition for controlling plant diseases comprising any one of the present compound T and fluindapyr at a ratio of 10:1;
Composition group MX25: a composition for controlling plant diseases comprising any one of the present compound T and pydiflumetofen at a ratio of 0.1:1;
Composition group MX26: a composition for controlling plant diseases comprising any one of the present compound T and pydiflumetofen at a ratio of 1:1;
Composition group MX27: a composition for controlling plant diseases comprising any one of the present compound T and pydiflumetofen at a ratio of 10:1;
Composition group MX28: a composition for controlling plant diseases comprising any one of the present compound T and 3-(difluoromethyl)-N-(2,3-dihydro-1,1,3-trimethyl-1H-inden-4-yl)-1-methyl-1H-pyrazol-4-carboxamide at a ratio of 0.1:1;
Composition group MX29: a composition for controlling plant diseases comprising any one of the present compound T and 3-(difluoromethyl)-N-(2,3-dihydro-1,1,3-trimethyl-1H-inden-4-yl)-1-methyl-1H-pyrazol-4-carboxamide at a ratio of 1:1;
Composition group MX30: a composition for controlling plant diseases comprising any one of the present compound T and 3-(difluoromethyl)-N-(2,3-dihydro-1,1,3-trimethyl-1H-inden-4-yl)-1-methyl-1H-pyrazol-4-carboxamide at a ratio of 10:1;
Composition group MX31: a composition for controlling plant diseases comprising any one of the present compound T and the compound represented by formula (1) at a ratio of 0.1:1;
Composition group MX32: a composition for controlling plant diseases comprising any one of the present compound T and the compound represented by formula (1) at a ratio of 1:1;
Composition group MX33: a composition for controlling plant diseases comprising any one of the present compound T and the compound represented by formula (1) at a ratio of 10:1;
Composition group MX34: a composition for controlling plant diseases comprising any one of the present compound T and the compound represented by formula (2) at a ratio of 0.1:1;
Composition group MX35: a composition for controlling plant diseases comprising any one of the present compound T and the compound represented by formula (2) at a ratio of 1:1;
Composition group MX36: a composition for controlling plant diseases comprising any one of the present compound T and the compound represented by formula (2) at a ratio of 10:1;
Composition group MX37: a composition for controlling plant diseases comprising any one of the present compound T and the compound represented by formula (3) at a ratio of 0.1:1;
Composition group MX38: a composition for controlling plant diseases comprising any one of the present compound T and the compound represented by formula (3) at a ratio of 1:1;
Composition group MX39: a composition for controlling plant diseases comprising any one of the present compound T and the compound represented by formula (3) at a ratio of 10:1;
Composition group MX40: a composition for controlling plant diseases comprising any one of the present compound T and mefentrifluconazole at a ratio of 0.1:1;
Composition group MX41: a composition for controlling plant diseases comprising any one of the present compound T and mefentrifluconazole at a ratio of 1:1;
Composition group MX42: a composition for controlling plant diseases comprising any one of the present compound T and mefentrifluconazole at a ratio of 10:1;
Composition group MX43: a composition for controlling plant diseases comprising any one of the present compound T and prothioconazole at a ratio of 0.1:1;
Composition group MX44: a composition for controlling plant diseases comprising any one of the present compound T and prothioconazole at a ratio of 1:1;
Composition group MX45: a composition for controlling plant diseases comprising any one of the present compound T and prothioconazole at a ratio of 10:1.
Composition group MX46: a composition for controlling plant diseases comprising any one of the present compound T and 3-(2-methylphenyl)butan-2-yl N-[(3-acetoxy-4-methoxypyridin-2-yl)carbonyl]alaninate at a ratio of 0.1:1.
Composition group MX47: a composition for controlling plant diseases comprising any one of the present compound T and 3-(2-methylphenyl)butan-2-yl N-[(3-acetoxy-4-methoxypyridin-2-yl)carbonyl]alaninate at a ratio of 1:1.
Composition group MX48: a composition for controlling plant diseases comprising any one of the present compound T and 3-(2-methylphenyl)butan-2-yl N-[(3-acetoxy-4-methoxypyridin-2-yl)carbonyl]alaninate at a ratio of 10:1.
Composition group MX49: a composition for controlling plant diseases comprising any one of the present compound T and 3-(4-fluoro-2-methylphenyl)butan-2-yl N-[(3-acetoxy-4-methoxypyridin-2-yl)carbonyl]alaninate at a ratio of 0.1:1.
Composition group MX50: a composition for controlling plant diseases comprising any one of the present compound T and 3-(4-fluoro-2-methylphenyl)butan-2-yl N-[(3-acetoxy-4-methoxypyridin-2-yl)carbonyl]alaninate at a ratio of 1:1.
Composition group MX51: a composition for controlling plant diseases comprising any one of the present compound T and 3-(4-fluoro-2-methylphenyl)butan-2-yl N-[(3-acetoxy-4-methoxypyridin-2-yl)carbonyl]alaninate at a ratio of 10:1.
Composition group MX52: a composition for controlling plant diseases comprising any one of the present compound T and 3-(4-methoxy-2-methylphenyl)butan-2-yl N-[(3-acetoxy-4-methoxypyridin-2-yl)carbonyl]alaninate at a ratio of 0.1:1.
Composition group MX53: a composition for controlling plant diseases comprising any one of the present compound T and 3-(4-methoxy-2-methylphenyl)butan-2-yl N-[(3-acetoxy-4-methoxypyridin-2-yl)carbonyl]alaninate at a ratio of 1:1.
Composition group MX54: a composition for controlling plant diseases comprising any one of the present compound T and 3-(4-methoxy-2-methylphenyl)butan-2-yl N-[(3-acetoxy-4-methoxypyridin-2-yl)carbonyl]alaninate at a ratio of 10:1.
Composition group MX55: a composition for controlling plant diseases comprising any one of the present compound T and 3-(2,4-dimethylphenyl)butan-2-yl N-[(3-acetoxy-4-methoxypyridin-2-yl)carbonyl]alaninate at a ratio of 0.1:1.
Composition group MX56: a composition for controlling plant diseases comprising any one of the present compound T and 3-(2,4-dimethylphenyl)butan-2-yl N-[(3-acetoxy-4-methoxypyridin-2-yl)carbonyl]alaninate at a ratio of 1:1.
Composition group MX57: a composition for controlling plant diseases comprising any one of the present compound T and 3-(2,4-dimethylphenyl)butan-2-yl N-[(3-acetoxy-4-methoxypyridin-2-yl)carbonyl]alaninate at a ratio of 10:1.
Composition group MX58: a composition for controlling plant diseases comprising any one of the present compound T and 3-(2-methylphenyl)butan-2-yl N-({3-[(2-methylpropanoyl)oxy]-4-methoxypyridin-2-yl}carbonyl)alaninate at a ratio of 0.1:1.
Composition group MX59: a composition for controlling plant diseases comprising any one of the present compound T and 3-(2-methylphenyl)butan-2-yl N-({3-[(2-methylpropanoyl)oxy]-4-methoxypyridin-2-yl}carbonyl)alaninate at a ratio of 1:1.
Composition group MX60: a composition for controlling plant diseases comprising any one of the present compound T and 3-(2-methylphenyl)butan-2-yl N-({3-[(2-methylpropanoyl)oxy]-4-methoxypyridin-2-yl}carbonyl)alaninate at a ratio of 10:1.
Composition group MX61: a composition for controlling plant diseases comprising any one of the present compound T and 3-(4-fluoro-2-methylphenyl)butan-2-yl N-({3-[(2-methylpropanoyl)oxy]-4-methoxypyridin-2-yl}carbonyl)alaninate at a ratio of 0.1:1.
Composition group MX62: a composition for controlling plant diseases comprising any one of the present compound T and 3-(4-fluoro-2-methylphenyl)butan-2-yl N-({3-[(2-methylpropanoyl)oxy]-4-methoxypyridin-2-yl}carbonyl)alaninate at a ratio of 1:1.
Composition group MX63: a composition for controlling plant diseases comprising any one of the present compound T and 3-(4-fluoro-2-methylphenyl)butan-2-yl N-({3-[(2-methylpropanoyl)oxy]-4-methoxypyridin-2-yl}carbonyl)alaninate at a ratio of 10:1.
Composition group MX64: a composition for controlling plant diseases comprising any one of the present compound T and 3-(4-methoxy-2-methylphenyl)butan-2-yl N-({3-[(2-methylpropanoyl)oxy]-4-methoxypyridin-2-yl}carbonyl)alaninate at a ratio of 0.1:1.
Composition group MX65: a composition for controlling plant diseases comprising any one of the present compound T and 3-(4-methoxy-2-methylphenyl)butan-2-yl N-({3-[(2-methylpropanoyl)oxy]-4-methoxypyridin-2-yl}carbonyl)alaninate at a ratio of 1:1.
Composition group MX66: a composition for controlling plant diseases comprising any one of the present compound T and 3-(4-methoxy-2-methylphenyl)butan-2-yl N-({3-[(2-methylpropanoyl)oxy]-4-methoxypyridin-2-yl}carbonyl)alaninate at a ratio of 10:1.
Composition group MX67: a composition for controlling plant diseases comprising any one of the present compound T and 3-(2,4-dimethylphenyl)butan-2-yl N-({3-[(2-methylpropanoyl)oxy]-4-methoxypyridin-2-yl}carbonyl)alaninate at a ratio of 0.1:1.
Composition group MX68: a composition for controlling plant diseases comprising any one of the present compound T and 3-(2,4-dimethylphenyl)butan-2-yl N-({3-[(2-methylpropanoyl)oxy]-4-methoxypyridin-2-yl}carbonyl)alaninate at a ratio of 1:1.
Composition group MX69: a composition for controlling plant diseases comprising any one of the present compound T and 3-(2,4-dimethylphenyl)butan-2-yl N-({3-[(2-methylpropanoyl)oxy]-4-methoxypyridin-2-yl}carbonyl)alaninate at a ratio of 10:1.
Composition group MX70: a composition for controlling plant diseases comprising any one of the present compound T and 3-(2-methylphenyl)butan-2-yl N-[(3-hydroxy-4-methoxypyridin-2-yl)carbonyl]alaninate at a ratio of 0.1:1.
Composition group MX71: a composition for controlling plant diseases comprising any one of the present compound T and 3-(2-methylphenyl)butan-2-yl N-[(3-hydroxy-4-methoxypyridin-2-yl)carbonyl]alaninate at a ratio of 1:1.
Composition group MX72: a composition for controlling plant diseases comprising any one of the present compound T and 3-(2-methylphenyl)butan-2-yl N-[(3-hydroxy-4-methoxypyridin-2-yl)carbonyl]alaninate at a ratio of 10:1.
Composition group MX73: a composition for controlling plant diseases comprising any one of the present compound T and 3-(4-fluoro-2-methylphenyl)butan-2-yl N-[(3-hydroxy-4-methoxypyridin-2-yl)carbonyl]alaninate at a ratio of 0.1:1.
Composition group MX74: a composition for controlling plant diseases comprising any one of the present compound T and 3-(4-fluoro-2-methylphenyl)butan-2-yl N-[(3-hydroxy-4-methoxypyridin-2-yl)carbonyl]alaninate at a ratio of 1:1.
Composition group MX75: a composition for controlling plant diseases comprising any one of the present compound T and 3-(4-fluoro-2-methylphenyl)butan-2-yl N-[(3-hydroxy-4-methoxypyridin-2-yl)carbonyl]alaninate at a ratio of 10:1.
Composition group MX76: a composition for controlling plant diseases comprising any one of the present compound T and 3-(4-methoxy-2-methylphenyl)butan-2-yl N-[(3-hydroxy-4-methoxypyridin-2-yl)carbonyl]alaninate at a ratio of 0.1:1.
Composition group MX77: a composition for controlling plant diseases comprising any one of the present compound T and 3-(4-methoxy-2-methylphenyl)butan-2-yl N-[(3-hydroxy-4-methoxypyridin-2-yl)carbonyl]alaninate at a ratio of 1:1.
Composition group MX78: a composition for controlling plant diseases comprising any one of the present compound T and 3-(4-methoxy-2-methylphenyl)butan-2-yl N-[(3-hydroxy-4-methoxypyridin-2-yl)carbonyl]alaninate at a ratio of 10:1.
Composition group MX79: a composition for controlling plant diseases comprising any one of the present compound T and 3-(2,4-dimethylphenyl)butan-2-yl N-[(3-hydroxy-4-methoxypyridin-2-yl)carbonyl]alaninate at a ratio of 0.1:1.
Composition group MX80: a composition for controlling plant diseases comprising any one of the present compound T and 3-(2,4-dimethylphenyl)butan-2-yl N-[(3-hydroxy-4-methoxypyridin-2-yl)carbonyl]alaninate at a ratio of 1:1.
Composition group MX81: a composition for controlling plant diseases comprising any one of the present compound T and 3-(2,4-dimethylphenyl)butan-2-yl N-[(3-hydroxy-4-methoxypyridin-2-yl)carbonyl]alaninate at a ratio of 10:1.
Composition group MX82: a composition for controlling plant diseases comprising any one of the present compound T and (2S,3S)-3-(2-methylphenyl)butan-2-yl N-[(3-acetoxy-4-methoxypyridin-2-yl)carbonyl]-L-alaninate at a ratio of 0.1:1.
Composition group MX83: a composition for controlling plant diseases comprising any one of the present compound T and (2S,3S)-3-(2-methylphenyl)butan-2-yl N-[(3-acetoxy-4-methoxypyridin-2-yl)carbonyl]-L-alaninate at a ratio of 1:1.
Composition group MX84: a composition for controlling plant diseases comprising any one of the present compound T and (2S,3S)-3-(2-methylphenyl)butan-2-yl N-[(3-acetoxy-4-methoxypyridin-2-yl)carbonyl]-L-alaninate at a ratio of 10:1.
Composition group MX85: a composition for controlling plant diseases comprising any one of the present compound T and (2S,3S)-3-(4-fluoro-2-methylphenyl)butan-2-yl N-[(3-acetoxy-4-methoxypyridin-2-yl)carbonyl]-L-alaninate at a ratio of 0.1:1.
Composition group MX86: a composition for controlling plant diseases comprising any one of the present compound T and (2S,3S)-3-(4-fluoro-2-methylphenyl)butan-2-yl N-[(3-acetoxy-4-methoxypyridin-2-yl)carbonyl]-L-alaninate at a ratio of 1:1.
Composition group MX87: a composition for controlling plant diseases comprising any one of the present compound T and (2S,3S)-3-(4-fluoro-2-methylphenyl)butan-2-yl N-[(3-acetoxy-4-methoxypyridin-2-yl)carbonyl]-L-alaninate at a ratio of 10:1.
Composition group MX88: a composition for controlling plant diseases comprising any one of the present compound T and (2S,3S)-3-(4-methoxy-2-methylphenyl)butan-2-yl N-[(3-acetoxy-4-methoxypyridin-2-yl)carbonyl]-L-alaninate at a ratio of 0.1:1.
Composition group MX89: a composition for controlling plant diseases comprising any one of the present compound T and (2S,3S)-3-(4-methoxy-2-methylphenyl)butan-2-yl N-[(3-acetoxy-4-methoxypyridin-2-yl)carbonyl]-L-alaninate at a ratio of 1:1.
Composition group MX90: a composition for controlling plant diseases comprising any one of the present compound T and (2S,3S)-3-(4-methoxy-2-methylphenyl)butan-2-yl N-[(3-acetoxy-4-methoxypyridin-2-yl)carbonyl]-L-alaninate at a ratio of 10:1.
Composition group MX91: a composition for controlling plant diseases comprising any one of the present compound T and (2S,3S)-3-(2,4-dimethylphenyl)butan-2-yl N-[(3-acetoxy-4-methoxypyridin-2-yl)carbonyl]-L-alaninate at a ratio of 0.1:1.
Composition group MX92: a composition for controlling plant diseases comprising any one of the present compound T and (2S,3S)-3-(2,4-dimethylphenyl)butan-2-yl N-[(3-acetoxy-4-methoxypyridin-2-yl)carbonyl]-L-alaninate at a ratio of 1:1.
Composition group MX93: a composition for controlling plant diseases comprising any one of the present compound T and (2S,3S)-3-(2,4-dimethylphenyl)butan-2-yl N-[(3-acetoxy-4-methoxypyridin-2-yl)carbonyl]-L-alaninate at a ratio of 10:1.
Composition group MX94: a composition for controlling plant diseases comprising any one of the present compound T and (2S,3S)-3-(2-methylphenyl)butan-2-yl N-({3-[(2-methylpropanoyl)oxy]-4-methoxypyridin-2-yl}carbonyl)-L-alaninate at a ratio of 0.1:1.
Composition group MX95: a composition for controlling plant diseases comprising any one of the present compound T and (2S,3S)-3-(2-methylphenyl)butan-2-yl N-({3-[(2-methylpropanoyl)oxy]-4-methoxypyridin-2-yl}carbonyl)-L-alaninate at a ratio of 1:1.
Composition group MX96: a composition for controlling plant diseases comprising any one of the present compound T and (2S,3S)-3-(2-methylphenyl)butan-2-yl N-({3-[(2-methylpropanoyl)oxy]-4-methoxypyridin-2-yl}carbonyl)-L-alaninate at a ratio of 10:1.
Composition group MX97: a composition for controlling plant diseases comprising any one of the present compound T and (2S,3S)-3-(4-fluoro-2-methylphenyl)butan-2-yl N-({3-[(2-methylpropanoyl)oxy]-4-methoxypyridin-2-yl}carbonyl)-L-alaninate at a ratio of 0.1:1.
Composition group MX98: a composition for controlling plant diseases comprising any one of the present compound T and (2S,3S)-3-(4-fluoro-2-methylphenyl)butan-2-yl N-({3-[(2-methylpropanoyl)oxy]-4-methoxypyridin-2-yl}carbonyl)-L-alaninate at a ratio of 1:1.
Composition group MX99: a composition for controlling plant diseases comprising any one of the present compound T and (2S,3S)-3-(4-fluoro-2-methylphenyl)butan-2-yl N-({3-[(2-methylpropanoyl)oxy]-4-methoxypyridin-2-yl}carbonyl)-L-alaninate at a ratio of 10:1.
Composition group MX100: a composition for controlling plant diseases comprising any one of the present compound T and (2S,3S)-3-(4-methoxy-2-methylphenyl)butan-2-yl N-({3-[(2-methylpropanoyl)oxy]-4-methoxypyridin-2-yl}carbonyl)-L-alaninate at a ratio of 0.1:1.
Composition group MX101: a composition for controlling plant diseases comprising any one of the present compound T and (2S,3S)-3-(4-methoxy-2-methylphenyl)butan-2-yl N-({3-[(2-methylpropanoyl)oxy]-4-methoxypyridin-2-yl}carbonyl)-L-alaninate at a ratio of 1:1.
Composition group MX102: a composition for controlling plant diseases comprising any one of the present compound T and (2S,3S)-3-(4-methoxy-2-methylphenyl)butan-2-yl N-({3-[(2-methylpropanoyl)oxy]-4-methoxypyridin-2-yl}carbonyl)-L-alaninate at a ratio of 10:1.
Composition group MX103: a composition for controlling plant diseases comprising any one of the present compound T and (2S,3S)-3-(2,4-dimethylphenyl)butan-2-yl N-({3-[(2-methylpropanoyl)oxy]-4-methoxypyridin-2-yl}carbonyl)-L-alaninate at a ratio of 0.1:1.
Composition group MX104: a composition for controlling plant diseases comprising any one of the present compound T and (2S,3S)-3-(2,4-dimethylphenyl)butan-2-yl N-({3-[(2-methylpropanoyl)oxy]-4-methoxypyridin-2-yl}carbonyl)-L-alaninate at a ratio of 1:1.
Composition group MX105: a composition for controlling plant diseases comprising any one of the present compound T and (2S,3S)-3-(2,4-dimethylphenyl)butan-2-yl N-({3-[(2-methylpropanoyl)oxy]-4-methoxypyridin-2-yl}carbonyl)-L-alaninate at a ratio of 10:1.
Composition group MX106: a composition for controlling plant diseases comprising any one of the present compound T and (2S,3S)-3-(2-methylphenyl)butan-2-yl N-[(3-hydroxy-4-methoxypyridin-2-yl)carbonyl]-L-alaninate at a ratio of 0.1:1.
Composition group MX107: a composition for controlling plant diseases comprising any one of the present compound T and (2S,3S)-3-(2-methylphenyl)butan-2-yl N-[(3-hydroxy-4-methoxypyridin-2-yl)carbonyl]-L-alaninate at a ratio of 1:1.
Composition group MX108: a composition for controlling plant diseases comprising any one of the present compound T and (2S,3S)-3-(2-methylphenyl)butan-2-yl N-[(3-hydroxy-4-methoxypyridin-2-yl)carbonyl]-L-alaninate at a ratio of 10:1.
Composition group MX109: a composition for controlling plant diseases comprising any one of the present compound T and (2S,3S)-3-(4-fluoro-2-methylphenyl)butan-2-yl N-[(3-hydroxy-4-methoxypyridin-2-yl)carbonyl]-L-alaninate at a ratio of 0.1:1.
Composition group MX110: a composition for controlling plant diseases comprising any one of the present compound T and (2S,3S)-3-(4-fluoro-2-methylphenyl)butan-2-yl N-[(3-hydroxy-4-methoxypyridin-2-yl)carbonyl]-L-alaninate at a ratio of 1:1.
Composition group MX111: a composition for controlling plant diseases comprising any one of the present compound T and (2S,3S)-3-(4-fluoro-2-methylphenyl)butan-2-yl N-[(3-hydroxy-4-methoxypyridin-2-yl)carbonyl]-L-alaninate at a ratio of 10:1.
Composition group MX112: a composition for controlling plant diseases comprising any one of the present compound T and (2S,3S)-3-(4-methoxy-2-methylphenyl)butan-2-yl N-[(3-hydroxy-4-methoxypyridin-2-yl)carbonyl]-L-alaninate at a ratio of 0.1:1.
Composition group MX113: a composition for controlling plant diseases comprising any one of the present compound T and (2S,3S)-3-(4-methoxy-2-methylphenyl)butan-2-yl N-[(3-hydroxy-4-methoxypyridin-2-yl)carbonyl]-L-alaninate at a ratio of 1:1.
Composition group MX114: a composition for controlling plant diseases comprising any one of the present compound T and (2S,3S)-3-(4-methoxy-2-methylphenyl)butan-2-yl N-[(3-hydroxy-4-methoxypyridin-2-yl)carbonyl]-L-alaninate at a ratio of 10:1.
Composition group MX115: a composition for controlling plant diseases comprising any one of the present compound T and (2S,3S)-3-(2,4-dimethylphenyl)butan-2-yl N-[(3-hydroxy-4-methoxypyridin-2-yl)carbonyl]-L-alaninate at a ratio of 0.1:1.
Composition group MX116: a composition for controlling plant diseases comprising any one of the present compound T and (2S,3S)-3-(2,4-dimethylphenyl)butan-2-yl N-[(3-hydroxy-4-methoxypyridin-2-yl)carbonyl]-L-alaninate at a ratio of 1:1.
Composition group MX117: a composition for controlling plant diseases comprising any one of the present compound T and (2S,3S)-3-(2,4-dimethylphenyl)butan-2-yl N-[(3-hydroxy-4-methoxypyridin-2-yl)carbonyl]-L-alaninate at a ratio of 10:1.

Next, the formulation examples of the compounds described herein are shown below. In the formulation examples, the “parts” represents “part by weight” unless otherwise specified. The present compound S represents the compounds described in the Compound Classes SX1 to SX1720.

### Formulation Example 1

Fifty (50) parts of any one of the present compound S, 3 parts of calcium lignin sulfonate, 2 parts of sodium lauryl sulfate, and 45 parts of wet silica are well mixed-grinding to obtain a formulation.

### Formulation Exmaple 2

Twenty (20) parts of any one of the present compound S, 1.5 parts of sorbitan trioleate are mixed with 28.5 parts of an aqueous solution containing 2 parts of polyvinyl alcohol, and the mixture is then finely-ground by a wet grinding method. To the mixture is then added 40 parts of an aqueous solution containing 0.05 parts of xanthan gum and 0.1 parts of magnesium aluminum silicate, and 10 parts of propylene glycol is further added thereto. The mixture is stirred to obtain a formulation.

### Formulation Example 3

Two (2) parts of any one of the present compound S, 88 parts of kaolin clay and 10 parts of talc are mixed-grinding to obtain a formulation.

### Formulation Example 4

Five (5) parts of any one of the present compound S, 14 parts of polyoxyethylene styryl phenyl ether, 6 parts of calcium dodecylbenzene sulfonate and 75 parts of xylene are well mixed to obtain a formulation.

### Formulation Example 5

Five (5) parts of any one of the present compound S, 1 part of wet silica, 2 parts of calcium lignosulfonate, 30 parts of bentonite and 65 parts of kaolin clay are mixed-grinding, and thereto is added water, and the mixture is well kneaded and is then granulated and dried to obtain a formulation.

### Formulation Example 6

Thirty five (35) parts of a mixture of ammonium polyoxyethylene alkyl ether sulfate and wet silica (weight ratio: 1:1), 20 parts of any one of the present compound S, and 45 parts of water are well mixed to obtain a formulation.

Next, the formulation examples of the comositions of the present invention are shown below. In the formulation examples, the “parts” represents “part by weight” unless otherwise specified. The compound MX represents the compositions described in the Composition Classes MX1 to MX45.

### Formulation Example A1

Fifty (50) parts of any one of the composition MX, 3 parts of calcium lignin sulfonate, 2 parts of sodium lauryl sulfate, and 45 parts of wet silica are well mixed-grinding to obtain a formulation.

### Formulation Exmaple A2

Twenty (20) parts of any one of the composition MX, 1.5 parts of sorbitan trioleate are mixed with 28.5 parts of an aqueous solution containing 2 parts of polyvinyl alcohol, and the mixture is then finely-ground by a wet grinding method. To the mixture is then added 40 parts of an aqueous solution containing 0.05 parts of xanthan gum and 0.1 parts of magnesium aluminum silicate, and 10 parts of propylene glycol is further added thereto. The mixture is stirred to obtain a formulation.

### Formulation Example A3

Two (2) parts of any one of the composition MX, 88 parts of kaolin clay and 10 parts of talc are mixed-grinding to obtain a formulation.

### Formulation Example A4

Five (5) parts of any one of the composition MX, 14 parts of polyoxyethylene styryl phenyl ether, 6 parts of calcium dodecylbenzene sulfonate and 75 parts of xylene are well mixed to obtain a formulation.

### Formulation Example A5

Five (5) parts of any one of the composition MX, 1 part of wet silica, 2 parts of calcium lignosulfonate, 30 parts of bentonite and 65 parts of kaolin clay are mixed-grinding, and thereto is added water, and the mixture is well kneaded and is then granulated and dried to obtain a formulation.

### Formulation Example A6

Thirty five (35) parts of a mixture of ammonium polyoxyethylene alkyl ether sulfate and wet silica (weight ratio: 1:1), 20 parts of any one of the composition MX, and 45 parts of water are well mixed to obtain a formulation.

Next, Test Examples of the compounds described herein are described.

### Test Example 1

Soybean leaf (cv; Kurosengoku) was punched out to 1 cm diameter to prepare a leaf disk. Each 1 mL of an agar medium (agar concentration 1.2%) was dispensed in 24 well microplate. A piece of the leaf disk was placed on each well. To a mixture of 0.5 μL of Sorpol (registered trademark) 1200KX, 4.5 μL of DMSO, and 5 μL of xylene was added 20 μL of a solution containing 10000 ppm of the test compound in DMSO. The resulting mixture was diluted with ion exchange water to prepare a spray solution containing a predetermined concentration of the test compound. The spray solution was sprayed in 10 μL per one leaf disk. After 1 day, an aqueous suspension of spores of soybean rust fungus (Phakopsora pachyrhizi) having an amino acid substitution of F129L on mitochondrial cytochrome b protein (1.0×10⁵/mL) was inoculated onto the leaf disks. After the inoculation, the microplate was placed in a growth chamber (light on for 6 hours, light off for 18 hours, 23° C. temperature, 60% humidity). After 1 day, the leaf disks were air-dried to disappear water droplets on the surface of the leaf disk, and the microplate was placed again in the growth chamber for 12 days. Thereafter, a lesion area of soybean rust disease was assessed. As a result, in all cases, the lesion areas of the leaf disk on which any one of the present compounds 1-1, 1-2, 2-2, 2-4, 2-7, 2-8, 2-9, 2-10, 4-1, 4-2, 4-3, 4-4, 4-6, 4-8, 4-9, 4-10, 9-4, 9-6, 2-3, 2-5, 3-1, 3-2, 4-12, 4-13, 4-14, 4-15, 4-17, 4-27, 4-36, 4-37, 4-38, 4-39, 4-40, 4-41, 4-43, 4-44, 4-45, 4-46, 4-47, 4-48, 5-1, 10-1, 10-2, 11-2, 11-18, 12-1, 12-2, 13-1, 13-2, 13-3, 13-4, 14-1, 14-3, 1A-1, 1A-2, 1A-3, 2A-1, 1-3, 1-4, 1-5, 1-6, 1-8, 1-9, 1-10, 2-12, 2-14, 4-21, 4-22, 4-24, 4-28, 4-29, 4-42, 4-59, 4-60, 4-61, 4-62, 4-63, 4-64, 4-65, 4-66, 4-67, 4-68, 4-69, 4-70, 4-73, 4-74, 4-76, 4-78, 4-79, 4-80, 4-81, 5-2, 9-5, 10-4, 12-3, 14-4, 14-5, 14-6, 14-7, 14-8, 14-9, 14-10, 14-11, 14-12, 14-13, 14-14, 14-17, 14-25, 14-26, 14-27, 14-28, 14-29, 14-30, 14-31, 14-32, 14-33, 14-34, 14-44, 2A-4, 3A-1 or 4A-1 was applied as a test compound at 50 ppm was shown to 30% or less of the lesion area of the untreated leaf disk. Here the untreated means that the spray solution containing the test compound was not sprayed onto the leaf disk.

### Comparative Test Example 1

The test was conducted by using as a test compound the present compound 1-1, 1-2, 2-2, 2-4, 2-8, 4-2, 4-3, 14-6, or 14-8, or pyraclostrobin, azoxystrobin, picoxystrobin, orysastrobin, dimoxystrobin, metominostrobin, or pyribencarb at 12.55 ppm or 3.1 ppm according to a method described in the Test Example 1. The results are indicated in [Table A], and [Table B].

TABLE A

| | | Lesion Area at each of | |
|---|---|---|---|
| Compound | | concentrations (%) | |
| No. | Structural formula | 12.5 ppm | 3.1 ppm |
| 1-1 | 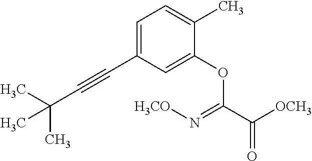 [SMILES: CO/N=C(\Oc1cc(C#CC(C)(C)C)ccc1C)C(=O)OC] | 0 | 10 |
| 1-2 | 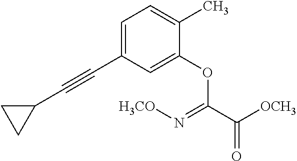 [SMILES: CO/N=C(\Oc1cc(C#CC2CC2)ccc1C)C(=O)OC] | 0 | 10 |
| 2-2 | 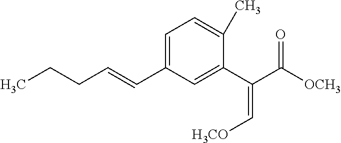 [SMILES: CCC/C=C/c1ccc(C)c(/C(=C\OC)C(=O)OC)c1] | 10 | 30 |
| 2-4 | 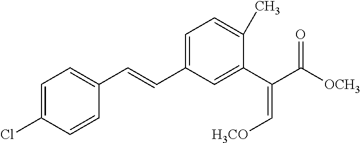 [SMILES: CO/C=C(/C(=O)OC)c1cc(/C=C/c2ccc(Cl)cc2)ccc1C] | 0 | 10 |
| 2-8 | 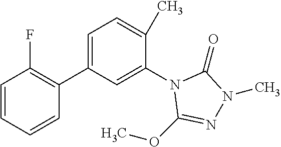 [SMILES: COc1nn(C)c(=O)n1-c1cc(-c2ccccc2F)ccc1C] | 0 | 10 |
| 4-2 | 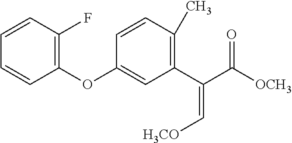 [SMILES: CO/C=C(/C(=O)OC)c1cc(Oc2ccccc2F)ccc1C] | 0 | 0 |
| 4-3 | 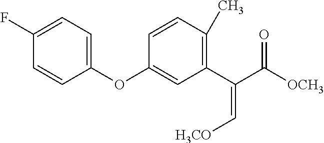 [SMILES: CO/C=C(/C(=O)OC)c1cc(Oc2ccc(F)cc2)ccc1C] | 0 | 0 |
| 14-6 | 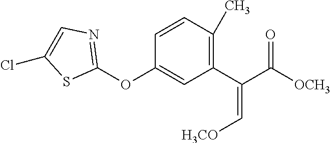 [SMILES: CO/C=C(/C(=O)OC)c1cc(Oc2ncc(Cl)s2)ccc1C] | 0 | 10 |
| 14-8 | 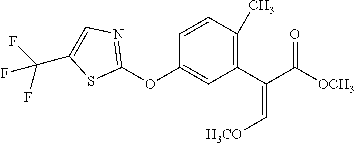 [SMILES: CO/C=C(/C(=O)OC)c1cc(Oc2ncc(C(F)(F)F)s2)ccc1C] | 0 | 10 |

TABLE B

| | Lesion Area at each of | |
|---|---|---|
| | concentrations (%) | |
| Compound | 12.5 ppm | 3.1 ppm |
| 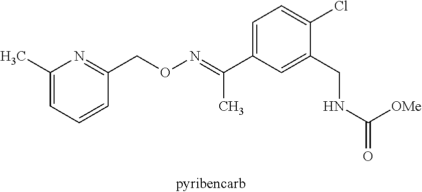 [SMILES: COC(=O)NCc1cc(/C(C)=N/OCc2cccc(C)n2)ccc1Cl] | 100 | 100 |
| 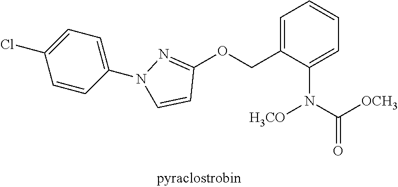 [SMILES: COC(=O)N(OC)c1ccccc1COc1ccn(-c2ccc(Cl)cc2)n1] | 100 | 100 |
| 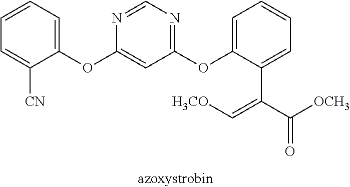 [SMILES: CO/C=C(/C(=O)OC)c1ccccc1Oc1cc(Oc2ccccc2C#N)ncn1] | 100 | 100 |
| 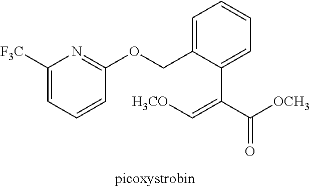 [SMILES: CO/C=C(/C(=O)OC)c1ccccc1COc1cccc(C(F)(F)F)n1] | 100 | 100 |
| 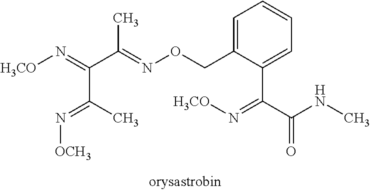 [SMILES: CNC(=O)/C(=N/OC)c1ccccc1CO/N=C(C)/C(=N/OC)C(/C)=N/OC] | 100 | 100 |
| 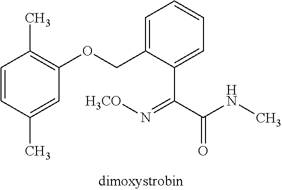 [SMILES: CNC(=O)/C(=N/OC)c1ccccc1COc1cc(C)ccc1C] | 100 | 100 |
| 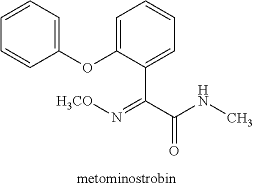 [SMILES: CNC(=O)/C(=N/OC)c1ccccc1Oc1ccccc1] | 100 | 100 |

The above-indicated results suggests that the present compound has superior efficacies for soybean rust fungus having an amino acid substitution of F129L in comparison with various commercially available QoI fungicides.

Next, the below-described test examples shows that the composition of the present invention is useful for controlling plant diseases.

### Test Example B1

Control Test Against Soybean Rust Disease (Phakopsora pachyrhizi)

Soybean leaf (cv; Kurosengoku) was punched out to 1 cm diameter to prepare a leaf disk. Each 1 mL of an agar medium (agar concentration 1.2%) was dispensed in 24 well microplate. A piece of the leaf disk was placed on each well. To a mixture of 1.0 μL of Sorpol (registered trademark) 1200KX, 4.5 μL of DMSO, and 5 μL of xylene was added 20 μL of a solution containing a predetermined concentration of a compound selected from the present compound T or 20 μL of a solution containing a predetermined concentration of the present compound B. The resulting mixture was diluted with ion exchange water to prepare a spray solution containing a predetermined concentration of the test compound. The agent solution was sprayed in 10 μL per one leaf disk. After 1 day, an aqueous suspension of spores of soybean rust fungus (Phakopsora pachyrhizi) (1.0×10⁵/mL) was inoculated onto the leaf disks. After the inoculation, the microplate was placed in a growth chamber (light on for 6 hours, light off for 18 hours, 23° C. temperature, 60% humidity). After 1 day, the leaf disks were air-dried to disappear water droplets on the surface of the leaf disk, and the microplate was placed again in the growth chamber for 12 days (which was “treated area”). Thereafter, a lesion area of soybean rust disease was assessed.

Control effect was calculated by the following “Equation 4” from the ratio of the lesion area of the incidence of disease in the treated area and the ratio of the lesion area of the incidence of disease in the non-treated area.

Control effect=100×(X−Y)/X “Equation 4”
X: Ratio of the lesion area of the incidence of disease in the non-treated area
Y: Ratio of the lesion area of the incidence of disease in the treated area

Here the non-treated area represents an area wherein the same procedures are taken except for using the present compound T and the present compound B.

The results are shown in [Table B1] to [Table B19].

TABLE B1

| | Conc. | Control |
|---|---|---|
| Present composition | (ppm) | Effect |
| Present compound 4-40 + benzovindiflupyr | 10 + 10 | 100 |
| Present compound 4-40 + benzovindiflupyr | 3 + 30 | 100 |
| Present compound 4-40 + benzovindiflupyr | 10 + 1 | 100 |
| Present compound 4-40 + 3-(difluoromethyl)-N-(2,3-dihydro-1,1,3- | 10 + 10 | 100 |
| trimethyl-1H-inden-4-yl)-1-methyl-1H-pyrazole-4- | | |
| carboxamide | | |
| Present compound 4-40 + 3-(difluoromethyl)-N-(2,3-dihydro-1,1,3- | 3 + 30 | 100 |
| trimethyl-1H-inden-4-yl)-1-methyl-1H-pyrazole-4- | | |
| carboxamide | | |
| Present compound 4-40 + 3-(difluoromethyl)-N-(2,3-dihydro-1,1,3- | 10 + 1 | 100 |
| trimethyl-1H-inden-4-yl)-1-methyl-1H-pyrazole-4- | | |
| carboxamide | | |
| Present compound 4-40 + fluindapyr | 10 + 10 | 100 |
| Present compound 4-40 + fluindapyr | 3 + 30 | 100 |
| Present compound 4-40 + fluindapyr | 10 + 1 | 100 |
| Present compound 4-40 + fluxapyroxad | 10 + 10 | 100 |
| Present compound 4-40 + fluxapyroxad | 3 + 30 | 100 |
| Present compound 4-40 + fluxapyroxad | 10 + 1 | 100 |
| Present compound 4-40 + Compound of formula (1) | 10 + 10 | 100 |
| Present compound 4-40 + Compound of formula (1) | 3 + 30 | 100 |
| Present compound 4-40 + Compound of formula (1) | 10 + 1 | 100 |
| Present compound 4-40 + Compound of formula (2) | 10 + 10 | 100 |
| Present compound 4-40 + Compound of formula (2) | 3 + 30 | 100 |
| Present compound 4-40 + Compound of formula (2) | 10 + 1 | 100 |
| Present compound 4-40 + Compound of formula (3) | 10 + 10 | 100 |
| Present compound 4-40 + Compound of formula (3) | 3 + 30 | 100 |
| Present compound 4-40 + Compound of formula (3) | 10 + 1 | 100 |
| Present compound 4-40 + pyraclostrobin | 10 + 10 | 100 |
| Present compound 4-40 + pyraclostrobin | 5 + 50 | 100 |
| Present compound 4-40 + pyraclostrobin | 10 + 1 | 100 |
| Present compound 4-40 + picoxystrobin | 10 + 10 | 100 |
| Present compound 4-40 + picoxystrobin | 5 + 50 | 100 |
| Present compound 4-40 + picoxystrobin | 10 + 1 | 100 |
| Present compound 4-40 + prothioconazole | 10 + 10 | 100 |
| Present compound 4-40 prothioconazole | 5 + 50 | 100 |
| Present compound 4-40 prothioconazole | 30 + 10 | 100 |
| Present compound 4-40 + fenpicoxamid | 10 + 10 | 100 |
| Present compound 4-40 + fenpicoxamid | 5 + 50 | 100 |
| Present compound 4-40 + fenpicoxamid | 30 + 10 | 100 |
| Present compound 4-40 + florylpicoxamid | 10 + 10 | 100 |
| Present compound 4-40 + florylpicoxamid | 5 + 50 | 100 |
| Present compound 4-40 + florylpicoxamid | 30 + 10 | 100 |
| Present compound 4-40 + 3-(2-methylphenyl)butan-2-yl N-[(3-hydroxy-4- | 10 + 10 | 100 |
| methoxypyridin-2-yl)carbonyl]alaninate | | |
| Present compound 4-40 + 3-(2-methylphenyl)butan-2-yl N-[(3-hydroxy-4- | 5 + 50 | 100 |
| methoxypyridin-2-yl)carbonyl]alaninate | | |
| Present compound 4-40 + 3-(2-methylphenyl)butan-2-yl N-[(3-hydroxy-4- | 10 + 1 | 100 |
| methoxypyridin-2-yl)carbonyl]alaninate | | |
| Present compound 4-40 + 3-(2,4-dimethylphenyl)butan-2-yl N-[(3-acetoxy-4- | 10 + 10 | 100 |
| methoxypyridin-2-yl)carbonyl]alaninate | | |
| Present compound 4-40 + 3-(2,4-dimethylphenyl)butan-2-yl N-[(3-acetoxy-4- | 5 + 50 | 100 |
| methoxypyridin-2-yl)carbonyl]alaninate | | |
| Present compound 4-40 + 3-(2,4-dimethylphenyl)butan-2-yl N-[(3-acetoxy-4- | 10 + 1 | 100 |
| methoxypyridin-2-yl)carbonyl]alaninate | | |
| Present compound 4-40 + 3-(4-fluoro-2-methylphenyl)butan-2-yl N-({3-[(2- | 10 + 10 | 100 |
| methylpropanoyl)oxy]-4-methoxypyridin-2- | | |
| yl}carbonyl)alaninate | | |
| Present compound 4-40 + 3-(4-fluoro-2-methylphenyl)butan-2-yl N-({3-[(2- | 5 + 50 | 100 |
| methylpropanoyl)oxy]-4-methoxypyridin-2- | | |
| yl}carbonyl)alaninate | | |
| Present compound 4-40 + 3-(4-fluoro-2-methylphenyl)butan-2-yl N-({3-[(2- | 10 + 1 | 100 |
| methylpropanoyl)oxy]-4-methoxypyridin-2- | | |
| yl}carbonyl)alaninate | | |
| Present compound 4-40 + 3-(4-methoxy-2-methylphenyl)butan-2-yl N-[(3- | 10 + 10 | 100 |
| hydroxy-4-methoxypyridin-2-yl)carbonyl]alaninate | | |
| Present compound 4-40 + 3-(4-methoxy-2-methylphenyl)butan-2-yl N-[(3- | 5 + 50 | 100 |
| hydroxy-4-methoxypyridin-2-yl)carbonyl]alaninate | | |
| Present compound 4-40 + 3-(4-methoxy-2-methylphenyl)butan-2-yl N-[(3- | 10 + 1 | 100 |
| hydroxy-4-methoxypyridin-2-yl)carbonyl]alaninate | | |

TABLE B2

| | Conc. | Control |
|---|---|---|
| Present composition | (ppm) | Effect |
| Present compound 4-17 + benzovindiflupyr | 10 + 10 | 100 |
| Present compound 4-17 + benzovindiflupyr | 3 + 30 | 100 |
| Present compound 4-17 + benzovindiflupyr | 10 + 1 | 100 |
| Present compound 4-17 + 3-(difluoromethyl)-N-(2,3-dihydro-1,1,3- | 10 + 10 | 100 |
| trimethyl-1H-inden-4-yl)-1-methyl-1H-pyrazole-4- | | |
| carboxamide | | |
| Present compound 4-17 + 3-(difluoromethyl)-N-(2,3-dihydro-1,1,3- | 3 + 30 | 100 |
| trimethyl-1H-inden-4-yl)-1-methyl-1H-pyrazole-4- | | |
| carboxamide | | |
| Present compound 4-17 + 3-(difluoromethyl)-N-(2,3-dihydro-1,1,3- | 10 + 1 | 100 |
| trimethyl-1H-inden-4-yl)-1-methyl-1H-pyrazole-4- | | |
| carboxamide | | |
| Present compound 4-17 + fluindapyr | 10 + 10 | 100 |
| Present compound 4-17 + fluindapyr | 3 + 30 | 100 |
| Present compound 4-17 + fluindapyr | 10 + 1 | 100 |
| Present compound 4-17 + fluxapyroxad | 10 + 10 | 100 |
| Present compound 4-17 + fluxapyroxad | 3 + 30 | 100 |
| Present compound 4-17 + fluxapyroxad | 10 + 1 | 100 |
| Present compound 4-17 + Compound of formula(1) | 10 + 10 | 100 |
| Present compound 4-17 + Compound of formula(1) | 3 + 30 | 100 |
| Present compound 4-17 + Compound of formula(1) | 10 + 1 | 100 |
| Present compound 4-17 + Compound of formula(2) | 10 + 10 | 100 |
| Present compound 4-17 + Compound of formula(2) | 3 + 30 | 100 |
| Present compound 4-17 + Compound of formula(2) | 10 + 1 | 100 |
| Present compound 4-17 + Compound of formula(3) | 10 + 10 | 100 |
| Present compound 4-17 + Compound of formula(3) | 3 + 30 | 100 |
| Present compound 4-17 + Compound of formula(3) | 10 + 1 | 100 |
| Present compound 4-17 + pyraclostrobin | 10 + 10 | 100 |
| Present compound 4-17 + pyraclostrobin | 5 + 50 | 100 |
| Present compound 4-17 + pyraclostrobin | 10 + 1 | 100 |
| Present compound 4-17 + picoxystrobin | 10 + 10 | 100 |
| Present compound 4-17 + picoxystrobin | 5 + 50 | 100 |
| Present compound 4-17 + picoxystrobin | 10 + 1 | 100 |
| Present compound 4-17 + prothioconazole | 10 + 10 | 100 |
| Present compound 4-17 + prothioconazole | 5 + 50 | 100 |
| Present compound 4-17 + prothioconazole | 30 + 10 | 100 |
| Present compound 4-17 + fenpicoxamid | 10 + 10 | 100 |
| Present compound 4-17 + fenpicoxamid | 5 + 50 | 100 |
| Present compound 4-17 + fenpicoxamid | 30 + 10 | 100 |
| Present compound 4-17 + florylpicoxamid | 10 + 10 | 100 |
| Present compound 4-17 + florylpicoxamid | 5 + 50 | 100 |
| Present compound 4-17 + florylpicoxamid | 30 + 10 | 100 |
| Present compound 4-17 + 3-(2-methylphenyl)butan-2-yl N-[(3-hydroxy-4- | 10 + 10 | 100 |
| methoxypyridin-2-yl)carbonyl]alaninate | | |
| Present compound 4-17 + 3-(2-methylphenyl)butan-2-yl N-[(3-hydroxy-4- | 5 + 50 | 100 |
| methoxypyridin-2-yl)carbonyl]alaninate | | |
| Present compound 4-17 + 3-(2-methylphenyl)butan-2-yl N-[(3-hydroxy-4- | 10 + 1 | 100 |
| methoxypyridin-2-yl)carbonyl]alaninate | | |
| Present compound 4-17 + 3-(2,4-dimethylphenyl)butan-2-yl N-[(3-acetoxy-4- | 10 + 10 | 100 |
| methoxypyridin-2-yl)carbonyl]alaninate | | |
| Present compound 4-17 + 3-(2,4-dimethylphenyl)butan-2-yl N-[(3-acetoxy-4- | 5 + 50 | 100 |
| methoxypyridin-2-yl)carbonyl]alaninate | | |
| Present compound 4-17 + 3-(2,4-dimethylphenyl)butan-2-yl N-[(3-acetoxy-4- | 10 + 1 | 100 |
| methoxypyridin-2-yl)carbonyl]alaninate | | |
| Present compound 4-17 + 3-(4-fluoro-2-methylphenyl)butan-2-yl N-({3-[(2- | 10 + 10 | 100 |
| methylpropanoyl)oxy]-4-methoxypyridin-2- | | |
| yl}carbonyl)alaninate | | |
| Present compound 4-17 + 3-(4-fluoro-2-methylphenyl)butan-2-yl N-({3-[(2- | 5 + 50 | 100 |
| methylpropanoyl)oxy]-4-methoxypyridin-2- | | |
| yl}carbonyl)alaninate | | |
| Present compound 4-17 + 3-(4-fluoro-2-methylphenyl)butan-2-yl N-({3-[(2- | 10 + 1 | 100 |
| methylpropanoyl)oxy]-4-methoxypyridin-2- | | |
| yl}carbonyl)alaninate | | |
| Present compound 4-17 + 3-(4-methoxy-2-methylphenyl)butan-2-yl N-[(3- | 10 + 10 | 100 |
| hydroxy-4-methoxypyridin-2-yl)carbonyl]alaninate | | |
| Present compound 4-17 + 3-(4-methoxy-2-methylphenyl)butan-2-yl N-[(3- | 5 + 50 | 100 |
| hydroxy-4-methoxypyridin-2-yl)carbonyl]alaninate | | |
| Present compound 4-17 + 3-(4-methoxy-2-methylphenyl)butan-2-yl N-[(3- | 10 + 1 | 100 |
| hydroxy-4-methoxypyridin-2-yl)carbonyl]alaninate | | |

TABLE B3

| | Conc. | Control |
|---|---|---|
| Present composition | (ppm) | Effect |
| Present compound 4-1 + benzovindiflupyr | 10 + 10 | 100 |
| Present compound 4-1 + benzovindiflupyr | 3 + 30 | 100 |
| Present compound 4-1 + benzovindiflupyr | 10 + 1 | 100 |
| Present compound 4-1 + 3-(difluoromethyl)-N-(2,3-dihydro-1,1,3- | 10 + 10 | 100 |
| trimethyl-1H-inden-4-yl)-1-methyl-1H-pyrazole-4- | | |
| carboxamide | | |
| Present compound 4-1 + 3-(difluoromethyl)-N-(2,3-dihydro-1,1,3- | 3 + 30 | 100 |
| trimethyl-1H-inden-4-yl)-1-methyl-1H-pyrazole-4- | | |
| carboxamide | | |
| Present compound 4-1 + 3-(difluoromethyl)-N-(2,3-dihydro-1,1,3- | 10 + 1 | 100 |
| trimethyl-1H-inden-4-yl)-1-methyl-1H-pyrazole-4- | | |
| carboxamide | | |
| Present compound 4-1 + fluindapyr | 10 + 10 | 100 |
| Present compound 4-1 + fluindapyr | 3 + 30 | 100 |
| Present compound 4-1 + fluindapyr | 10 + 1 | 100 |
| Present compound 4-1 + fluxapyroxad | 10 + 10 | 100 |
| Present compound 4-1 + fluxapyroxad | 3 + 30 | 100 |
| Present compound 4-1 + fluxapyroxad | 10 + 1 | 100 |
| Present compound 4-1 + Compound of formula(1) | 10 + 10 | 100 |
| Present compound 4-1 + Compound of formula(1) | 3 + 30 | 100 |
| Present compound 4-1 + Compound of formula(1) | 10 + 1 | 100 |
| Present compound 4-1 + Compound of formula(2) | 10 + 10 | 100 |
| Present compound 4-1 + Compound of formula(2) | 3 + 30 | 100 |
| Present compound 4-1 + Compound of formula(2) | 10 + 1 | 100 |
| Present compound 4-1 + Compound of formula(3) | 10 + 10 | 100 |
| Present compound 4-1 + Compound of formula(3) | 3 + 30 | 100 |
| Present compound 4-1 + Compound of formula(3) | 10 + 1 | 100 |
| Present compound 4-1 + pyraclostrobin | 10 + 10 | 100 |
| Present compound 4-1 + pyraclostrobin | 5 + 50 | 100 |
| Present compound 4-1 + pyraclostrobin | 10 + 1 | 100 |
| Present compound 4-1 + picoxystrobin | 10 + 10 | 100 |
| Present compound 4-1 + picoxystrobin | 5 + 50 | 100 |
| Present compound 4-1 + picoxystrobin | 10 + 1 | 100 |
| Present compound 4-1 + prothioconazole | 10 + 10 | 100 |
| Present compound 4-1 + prothioconazole | 5 + 50 | 100 |
| Present compound 4-1 + prothioconazole | 30 + 10 | 100 |
| Present compound 4-1 + fenpicoxamid | 10 + 10 | 100 |
| Present compound 4-1 + fenpicoxamid | 5 + 50 | 100 |
| Present compound 4-1 + fenpicoxamid | 30 + 10 | 100 |
| Present compound 4-1 + florylpicoxamid | 10 + 10 | 100 |
| Present compound 4-1 + florylpicoxamid | 5 + 50 | 100 |
| Present compound 4-1 + florylpicoxamid | 30 + 10 | 100 |
| Present compound 4-1 + 3-(2-methylphenyl)butan-2-yl N-[(3-hydroxy-4- | 10 + 10 | 100 |
| methoxypyridin-2-yl)carbonyl]alaninate | | |
| Present compound 4-1 + 3-(2-methylphenyl)butan-2-yl N-[(3-hydroxy-4- | 5 + 50 | 100 |
| methoxypyridin-2-yl)carbonyl]alaninate | | |
| Present compound 4-1 + 3-(2-methylphenyl)butan-2-yl N-[(3-hydroxy-4- | 10 + 1 | 100 |
| methoxypyridin-2-yl)carbonyl]alaninate | | |
| Present compound 4-1 + 3-(2,4-dimethylphenyl)butan-2-yl N-[(3-acetoxy-4- | 10 + 10 | 100 |
| methoxypyridin-2-yl)carbonyl]alaninate | | |
| Present compound 4-1 + 3-(2,4-dimethylphenyl)butan-2-yl N-[(3-acetoxy-4- | 5 + 50 | 100 |
| methoxypyridin-2-yl)carbonyl]alaninate | | |
| Present compound 4-1 + 3-(2,4-dimethylphenyl)butan-2-yl N-[(3-acetoxy-4- | 10 + 1 | 100 |
| methoxypyridin-2-yl)carbonyl]alaninate | | |
| Present compound 4-1 + 3-(4-fluoro-2-methylphenyl)butan-2-yl N-({3-[(2- | 10 + 10 | 100 |
| methylpropanoyl)oxy]-4-methoxypyridin-2- | | |
| yl}carbonyl)alaninate | | |
| Present compound 4-1 + 3-(4-fluoro-2-methylphenyl)butan-2-yl N-({3-[(2- | 5 + 50 | 100 |
| methylpropanoyl)oxy]-4-methoxypyridin-2- | | |
| yl}carbonyl)alaninate | | |
| Present compound 4-1 + 3-(4-fluoro-2-methylphenyl)butan-2-yl N-({3-[(2- | 10 + 1 | 100 |
| methylpropanoyl)oxy]-4-methoxypyridin-2- | | |
| yl}carbonyl)alaninate | | |
| Present compound 4-1 + 3-(4-methoxy-2-methylphenyl)butan-2-yl N-[(3- | 10 + 10 | 100 |
| hydroxy-4-methoxypyridin-2-yl)carbonyl]alaninate | | |
| Present compound 4-1 + 3-(4-methoxy-2-methylphenyl)butan-2-yl N-[(3- | 5 + 50 | 100 |
| hydroxy-4-methoxypyridin-2-yl)carbonyl]alaninate | | |
| Present compound 4-1 + 3-(4-methoxy-2-methylphenyl)butan-2-yl N-[(3- | 10 + 1 | 100 |
| hydroxy-4-methoxypyridin-2-yl)carbonyl]alaninate | | |

TABLE B4

| | Conc. | Control |
|---|---|---|
| Present composition | (ppm) | Effect |
| Present compound 4-36 + benzovindiflupyr | 10 + 10 | 100 |
| Present compound 4-36 + benzovindiflupyr | 3 + 30 | 100 |
| Present compound 4-36 + benzovindiflupyr | 10 + 1 | 100 |
| Present compound 4-36 + 3-(difluoromethyl)-N-(2,3-dihydro-1,1,3- | 10 + 10 | 100 |
| trimethyl-1H-inden-4-yl)-1-methyl-1H-pyrazole-4- | | |
| carboxamide | | |
| Present compound 4-36 + 3-(difluoromethyl)-N-(2,3-dihydro-1,1,3- | 3 + 30 | 100 |
| trimethyl-1H-inden-4-yl)-1-methyl-1H-pyrazole-4- | | |
| carboxamide | | |
| Present compound 4-36 + 3-(difluoromethyl)-N-(2,3-dihydro-1,1,3- | 10 + 1 | 100 |
| trimethyl-1H-inden-4-yl)-1-methyl-1H-pyrazole-4- | | |
| carboxamide | | |
| Present compound 4-36 + fluindapyr | 10 + 10 | 100 |
| Present compound 4-36 + fluindapyr | 3 + 30 | 100 |
| Present compound 4-36 + fluindapyr | 10 + 1 | 100 |
| Present compound 4-36 + fluxapyroxad | 10 + 10 | 100 |
| Present compound 4-36 + fluxapyroxad | 3 + 30 | 100 |
| Present compound 4-36 + fluxapyroxad | 10 + 1 | 100 |
| Present compound 4-36 + Compound of formula(1) | 10 + 10 | 100 |
| Present compound 4-36 + Compound of formula(1) | 3 + 30 | 100 |
| Present compound 4-36 + Compound of formula(1) | 10 + 1 | 100 |
| Present compound 4-36 + Compound of formula(2) | 10 + 10 | 100 |
| Present compound 4-36 + Compound of formula(2) | 3 + 30 | 100 |
| Present compound 4-36 + Compound of formula(2) | 10 + 1 | 100 |
| Present compound 4-36 + Compound of formula(3) | 10 + 10 | 100 |
| Present compound 4-36 + Compound of formula(3) | 3 + 30 | 100 |
| Present compound 4-36 + Compound of formula(3) | 10 + 1 | 100 |
| Present compound 4-36 + pyraclostrobin | 10 + 10 | 100 |
| Present compound 4-36 + pyraclostrobin | 5 + 50 | 100 |
| Present compound 4-36 + pyraclostrobin | 10 + 1 | 100 |
| Present compound 4-36 + picoxystrobin | 10 + 10 | 100 |
| Present compound 4-36 + picoxystrobin | 5 + 50 | 100 |
| Present compound 4-36 + picoxystrobin | 10 + 1 | 100 |
| Present compound 4-36 + prothioconazole | 10 + 10 | 100 |
| Present compound 4-36 + prothioconazole | 5 + 50 | 100 |
| Present compound 4-36 + prothioconazole | 30 + 10 | 100 |
| Present compound 4-36 + fenpicoxamid | 10 + 10 | 100 |
| Present compound 4-36 + fenpicoxamid | 5 + 50 | 100 |
| Present compound 4-36 + fenpicoxamid | 30 + 10 | 100 |
| Present compound 4-36 + florylpicoxamid | 10 + 10 | 100 |
| Present compound 4-36 + florylpicoxamid | 5 + 50 | 100 |
| Present compound 4-36 + florylpicoxamid | 30 + 10 | 100 |
| Present compound 4-36 + 3-(2-methylphenyl)butan-2-yl N-[(3-hydroxy-4- | 10 + 10 | 100 |
| methoxypyridin-2-yl)carbonyl]alaninate | | |
| Present compound 4-36 + 3-(2-methylphenyl)butan-2-yl N-[(3-hydroxy-4- | 5 + 50 | 100 |
| methoxypyridin-2-yl)carbonyl]alaninate | | |
| Present compound 4-36 + 3-(2-methylphenyl)butan-2-yl N-[(3-hydroxy-4- | 10 + 1 | 100 |
| methoxypyridin-2-yl)carbonyl]alaninate | | |
| Present compound 4-36 + 3-(2,4-dimethylphenyl)butan-2-yl N-[(3-acetoxy-4- | 10 + 10 | 100 |
| methoxypyridin-2-yl)carbonyl]alaninate | | |
| Present compound 4-36 + 3-(2,4-dimethylphenyl)butan-2-yl N-[(3-acetoxy-4- | 5 + 50 | 100 |
| methoxypyridin-2-yl)carbonyl]alaninate | | |
| Present compound 4-36 + 3-(2,4-dimethylphenyl)butan-2-yl N-[(3-acetoxy-4- | 10 + 1 | 100 |
| methoxypyridin-2-yl)carbonyl]alaninate | | |
| Present compound 4-36 + 3-(4-fluoro-2-methylphenyl)butan-2-yl N-({3-[(2- | 10 + 10 | 100 |
| methylpropanoyl)oxy]-4-methoxypyridin-2- | | |
| yl}carbonyl)alaninate | | |
| Present compound 4-36 + 3-(4-fluoro-2-methylphenyl)butan-2-yl N-({3-[(2- | 5 + 50 | 100 |
| methylpropanoyl)oxy]-4-methoxypyridin-2- | | |
| yl}carbonyl)alaninate | | |
| Present compound 4-36 + 3-(4-fluoro-2-methylphenyl)butan-2-yl N-({3-[(2- | 10 + 1 | 100 |
| methylpropanoyl)oxy]-4-methoxypyridin-2- | | |
| yl}carbonyl)alaninate | | |
| Present compound 4-36 + 3-(4-methoxy-2-methylphenyl)butan-2-yl N-[(3- | 10 + 10 | 100 |
| hydroxy-4-methoxypyridin-2-yl)carbonyl]alaninate | | |
| Present compound 4-36 + 3-(4-methoxy-2-methylphenyl)butan-2-yl N-[(3- | 5 + 50 | 100 |
| hydroxy-4-methoxypyridin-2-yl)carbonyl]alaninate | | |
| Present compound 4-36 + 3-(4-methoxy-2-methylphenyl)butan-2-yl N-[(3- | 10 + 1 | 100 |
| hydroxy-4-methoxypyridin-2-yl)carbonyl]alaninate | | |

TABLE B5

| | Conc. | Control |
|---|---|---|
| Present composition | (ppm) | Effect |
| Present compound 4-6 + benzovindiflupyr | 10 + 10 | 100 |
| Present compound 4-6 + benzovindiflupyr | 3 + 30 | 100 |
| Present compound 4-6 + benzovindiflupyr | 10 + 1 | 100 |
| Present compound 4-6 + 3-(difluoromethyl)-N-(2,3-dihydro-1,1,3- | 10 + 10 | 100 |
| trimethyl-1H-inden-4-yl)-1-methyl-1H-pyrazole-4- | | |
| carboxamide | | |
| Present compound 4-6 + 3-(difluoromethyl)-N-(2,3-dihydro-1,1,3- | 3 + 30 | 100 |
| trimethyl-1H-inden-4-yl)-1-methyl-1H-pyrazole-4- | | |
| carboxamide | | |
| Present compound 4-6 + 3-(difluoromethyl)-N-(2,3-dihydro-1,1,3- | 10 + 1 | 100 |
| trimethyl-1H-inden-4-yl)-1-methyl-1H-pyrazole-4- | | |
| carboxamide | | |
| Present compound 4-6 + fluindapyr | 10 + 10 | 100 |
| Present compound 4-6 + fluindapyr | 3 + 30 | 100 |
| Present compound 4-6 + fluindapyr | 10 + 1 | 100 |
| Present compound 4-6 + fluxapyroxad | 10 + 10 | 100 |
| Present compound 4-6 + fluxapyroxad | 3 + 30 | 100 |
| Present compound 4-6 + fluxapyroxad | 10 + 1 | 100 |
| Present compound 4-6 + Compound of formula(1) | 10 + 10 | 100 |
| Present compound 4-6 + Compound of formula(1) | 3 + 30 | 100 |
| Present compound 4-6 + Compound of formula(1) | 10 + 1 | 100 |
| Present compound 4-6 + Compound of formula(2) | 10 + 10 | 100 |
| Present compound 4-6 + Compound of formula(2) | 3 + 30 | 100 |
| Present compound 4-6 + Compound of formula(2) | 10 + 1 | 100 |
| Present compound 4-6 + Compound of formula(3) | 10 + 10 | 100 |
| Present compound 4-6 + Compound of formula(3) | 3 + 30 | 100 |
| Present compound 4-6 + Compound of formula(3) | 10 + 1 | 100 |
| Present compound 4-6 + pyraclostrobin | 10 + 10 | 100 |
| Present compound 4-6 + pyraclostrobin | 5 + 50 | 100 |
| Present compound 4-6 + pyraclostrobin | 10 + 1 | 100 |
| Present compound 4-6 + picoxystrobin | 10 + 10 | 100 |
| Present compound 4-6 + picoxystrobin | 5 + 50 | 100 |
| Present compound 4-6 + picoxystrobin | 10 + 1 | 100 |
| Present compound 4-6 + prothioconazole | 10 + 10 | 100 |
| Present compound 4-6 prothioconazole | 5 + 50 | 100 |
| Present compound 4-6 prothioconazole | 30 + 10 | 100 |
| Present compound 4-6 + fenpicoxamid | 10 + 10 | 100 |
| Present compound 4-6 + fenpicoxamid | 5 + 50 | 100 |
| Present compound 4-6 + fenpicoxamid | 30 + 10 | 100 |
| Present compound 4-6 + florylpicoxamid | 10 + 10 | 100 |
| Present compound 4-6 + florylpicoxamid | 5 + 50 | 100 |
| Present compound 4-6 + florylpicoxamid | 30 + 10 | 100 |
| Present compound 4-6 + 3-(2-methylphenyl)butan-2-yl N-[(3-hydroxy-4- | 10 + 10 | 100 |
| methoxypyridin-2-yl)carbonyl]alaninate | | |
| Present compound 4-6 + 3-(2-methylphenyl)butan-2-yl N-[(3-hydroxy-4- | 5 + 50 | 100 |
| methoxypyridin-2-yl)carbonyl]alaninate | | |
| Present compound 4-6 + 3-(2-methylphenyl)butan-2-yl N-[(3-hydroxy-4- | 10 + 1 | 100 |
| methoxypyridin-2-yl)carbonyl]alaninate | | |
| Present compound 4-6 + 3-(2,4-dimethylphenyl)butan-2-yl N-[(3-acetoxy-4- | 10 + 10 | 100 |
| methoxypyridin-2-yl)carbonyl]alaninate | | |
| Present compound 4-6 + 3-(2,4-dimethylphenyl)butan-2-yl N-[(3-acetoxy-4- | 5 + 50 | 100 |
| methoxypyridin-2-yl)carbonyl]alaninate | | |
| Present compound 4-6 + 3-(2,4-dimethylphenyl)butan-2-yl N-[(3-acetoxy-4- | 10 + 1 | 100 |
| methoxypyridin-2-yl)carbonyl]alaninate | | |
| Present compound 4-6 + 3-(4-fluoro-2-methylphenyl)butan-2-yl N-({3-[(2- | 10 + 10 | 100 |
| methylpropanoyl)oxy]-4-methoxypyridin-2- | | |
| yl}carbonyl)alaninate | | |
| Present compound 4-6 + 3-(4-fluoro-2-methylphenyl)butan-2-yl N-({3-[(2- | 5 + 50 | 100 |
| methylpropanoyl)oxy]-4-methoxypyridin-2- | | |
| yl}carbonyl)alaninate | | |
| Present compound 4-6 + 3-(4-fluoro-2-methylphenyl)butan-2-yl N-({3-[(2- | 10 + 1 | 100 |
| methylpropanoyl)oxy]-4-methoxypyridin-2- | | |
| yl}carbonyl)alaninate | | |
| Present compound 4-6 + 3-(4-methoxy-2-methylphenyl)butan-2-yl N-[(3- | 10 + 10 | 100 |
| hydroxy-4-methoxypyridin-2-yl)carbonyl]alaninate | | |
| Present compound 4-6 + 3-(4-methoxy-2-methylphenyl)butan-2-yl N-[(3- | 5 + 50 | 100 |
| hydroxy-4-methoxypyridin-2-yl)carbonyl]alaninate | | |
| Present compound 4-6 + 3-(4-methoxy-2-methylphenyl)butan-2-yl N-[(3- | 10 + 1 | 100 |
| hydroxy-4-methoxypyridin-2-yl)carbonyl]alaninate | | |

TABLE B6

| | Conc. | Control |
|---|---|---|
| Present composition | (ppm) | Effect |
| Present compound 4-38 + benzovindiflupyr | 10 + 10 | 100 |
| Present compound 4-38 + benzovindiflupyr | 3 + 30 | 100 |
| Present compound 4-38 + benzovindiflupyr | 10 + 1 | 100 |
| Present compound 4-38 + 3-(difluoromethyl)-N-(2,3-dihydro-1,1,3- | 10 + 10 | 100 |
| trimethyl-1H-inden-4-yl)-1-methyl-1H-pyrazole-4- | | |
| carboxamide | | |
| Present compound 4-38 + 3-(difluoromethyl)-N-(2,3-dihydro-1,1,3- | 3 + 30 | 100 |
| trimethyl-1H-inden-4-yl)-1-methyl-1H-pyrazole-4- | | |
| carboxamide | | |
| Present compound 4-38 + 3-(difluoromethyl)-N-(2,3-dihydro-1,1,3- | 10 + 1 | 100 |
| trimethyl-1H-inden-4-yl)-1-methyl-1H-pyrazole-4- | | |
| carboxamide | | |
| Present compound 4-38 + fluindapyr | 10 + 10 | 100 |
| Present compound 4-38 + fluindapyr | 3 + 30 | 100 |
| Present compound 4-38 + fluindapyr | 10 + 1 | 100 |
| Present compound 4-38 + fluxapyroxad | 10 + 10 | 100 |
| Present compound 4-38 + fluxapyroxad | 3 + 30 | 100 |
| Present compound 4-38 + fluxapyroxad | 10 + 1 | 100 |
| Present compound 4-38 + Compound of formula(1) | 10 + 10 | 100 |
| Present compound 4-38 + Compound of formula(1) | 3 + 30 | 100 |
| Present compound 4-38 + Compound of formula(1) | 10 + 1 | 100 |
| Present compound 4-38 + Compound of formula(2) | 10 + 10 | 100 |
| Present compound 4-38 + Compound of formula(2) | 3 + 30 | 100 |
| Present compound 4-38 + Compound of formula(2) | 10 + 1 | 100 |
| Present compound 4-38 + Compound of formula(3) | 10 + 10 | 100 |
| Present compound 4-38 + Compound of formula(3) | 3 + 30 | 100 |
| Present compound 4-38 + Compound of formula(3) | 10 + 1 | 100 |
| Present compound 4-38 + pyraclostrobin | 10 + 10 | 100 |
| Present compound 4-38 + pyraclostrobin | 5 + 50 | 100 |
| Present compound 4-38 + pyraclostrobin | 10 + 1 | 100 |
| Present compound 4-38 + picoxystrobin | 10 + 10 | 100 |
| Present compound 4-38 + picoxystrobin | 5 + 50 | 100 |
| Present compound 4-38 + picoxystrobin | 10 + 1 | 100 |
| Present compound 4-38 + prothioconazole | 10 + 10 | 100 |
| Present compound 4-38 + prothioconazole | 5 + 50 | 100 |
| Present compound 4-38 + prothioconazole | 30 + 10 | 100 |
| Present compound 4-38 + fenpicoxamid | 10 + 10 | 100 |
| Present compound 4-38 + fenpicoxamid | 5 + 50 | 100 |
| Present compound 4-38 + fenpicoxamid | 30 + 10 | 100 |
| Present compound 4-38 + florylpicoxamid | 10 + 10 | 100 |
| Present compound 4-38 + florylpicoxamid | 5 + 50 | 100 |
| Present compound 4-38 + florylpicoxamid | 30 + 10 | 100 |
| Present compound 4-38 + 3-(2-methylphenyl)butan-2-yl N-[(3-hydroxy-4- | 10 + 10 | 100 |
| methoxypyridin-2-yl)carbonyl]alaninate | | |
| Present compound 4-38 + 3-(2-methylphenyl)butan-2-yl N-[(3-hydroxy-4- | 5 + 50 | 100 |
| methoxypyridin-2-yl)carbonyl]alaninate | | |
| Present compound 4-38 + 3-(2-methylphenyl)butan-2-yl N-[(3-hydroxy-4- | 10 + 1 | 100 |
| methoxypyridin-2-yl)carbonyl]alaninate | | |
| Present compound 4-38 + 3-(2,4-dimethylphenyl)butan-2-yl N-[(3-acetoxy-4- | 10 + 10 | 100 |
| methoxypyridin-2-yl)carbonyl]alaninate | | |
| Present compound 4-38 + 3-(2,4-dimethylphenyl)butan-2-yl N-[(3-acetoxy-4- | 5 + 50 | 100 |
| methoxypyridin-2-yl)carbonyl]alaninate | | |
| Present compound 4-38 + 3-(2,4-dimethylphenyl)butan-2-yl N-[(3-acetoxy-4- | 10 + 1 | 100 |
| methoxypyridin-2-yl)carbonyl]alaninate | | |
| Present compound 4-38 + 3-(4-fluoro-2-methylphenyl)butan-2-yl N-({3-[(2- | 10 + 10 | 100 |
| methylpropanoyl)oxy]-4-methoxypyridin-2- | | |
| yl}carbonyl)alaninate | | |
| Present compound 4-38 + 3-(4-fluoro-2-methylphenyl)butan-2-yl N-({3-[(2- | 5 + 50 | 100 |
| methylpropanoyl)oxy]-4-methoxypyridin-2- | | |
| yl}carbonyl)alaninate | | |
| Present compound 4-38 + 3-(4-fluoro-2-methylphenyl)butan-2-yl N-({3-[(2- | 10 + 1 | 100 |
| methylpropanoyl)oxy]-4-methoxypyridin-2- | | |
| yl}carbonyl)alaninate | | |
| Present compound 4-38 + 3-(4-methoxy-2-methylphenyl)butan-2-yl N-[(3- | 10 + 10 | 100 |
| hydroxy-4-methoxypyridin-2-yl)carbonyl]alaninate | | |
| Present compound 4-38 + 3-(4-methoxy-2-methylphenyl)butan-2-yl N-[(3- | 5 + 50 | 100 |
| hydroxy-4-methoxypyridin-2-yl)carbonyl]alaninate | | |
| Present compound 4-38 + 3-(4-methoxy-2-methylphenyl)butan-2-yl N-[(3- | 10 + 1 | 100 |
| hydroxy-4-methoxypyridin-2-yl)carbonyl]alaninate | | |

TABLE B7

| | Conc. | Control |
|---|---|---|
| Present composition | (ppm) | Effect |
| Present compound 4-12 + benzovindiflupyr | 10 + 10 | 100 |
| Present compound 4-12 + benzovindiflupyr | 3 + 30 | 100 |
| Present compound 4-12 + benzovindiflupyr | 10 + 1 | 100 |
| Present compound 4-12 + 3-(difluoromethyl)-N-(2,3-dihydro-1,1,3- | 10 + 10 | 100 |
| trimethyl-1H-inden-4-yl)-1-methyl-1H-pyrazole-4- | | |
| carboxamide | | |
| Present compound 4-12 + 3-(difluoromethyl)-N-(2,3-dihydro-1,1,3- | 3 + 30 | 100 |
| trimethyl-1H-inden-4-yl)-1-methyl-1H-pyrazole-4- | | |
| carboxamide | | |
| Present compound 4-12 + 3-(difluoromethyl)-N-(2,3-dihydro-1,1,3- | 10 + 1 | 100 |
| trimethyl-1H-inden-4-yl)-1-methyl-1H-pyrazole-4- | | |
| carboxamide | | |
| Present compound 4-12 + fluindapyr | 10 + 10 | 100 |
| Present compound 4-12 + fluindapyr | 3 + 30 | 100 |
| Present compound 4-12 + fluindapyr | 10 + 1 | 100 |
| Present compound 4-12 + fluxapyroxad | 10 + 10 | 100 |
| Present compound 4-12 + fluxapyroxad | 3 + 30 | 100 |
| Present compound 4-12 + fluxapyroxad | 10 + 1 | 100 |
| Present compound 4-12 + Compound of formula(1) | 10 + 10 | 100 |
| Present compound 4-12 + Compound of formula(1) | 3 + 30 | 100 |
| Present compound 4-12 + Compound of formula(1) | 10 + 1 | 100 |
| Present compound 4-12 + Compound of formula(2) | 10 + 10 | 100 |
| Present compound 4-12 + Compound of formula(2) | 3 + 30 | 100 |
| Present compound 4-12 + Compound of formula(2) | 10 + 1 | 100 |
| Present compound 4-12 + Compound of formula(3) | 10 + 10 | 100 |
| Present compound 4-12 + Compound of formula(3) | 3 + 30 | 100 |
| Present compound 4-12 + Compound of formula(3) | 10 + 1 | 100 |
| Present compound 4-12 + pyraclostrobin | 10 + 10 | 100 |
| Present compound 4-12 + pyraclostrobin | 5 + 50 | 100 |
| Present compound 4-12 + pyraclostrobin | 10 + 1 | 100 |
| Present compound 4-12 + picoxystrobin | 10 + 10 | 100 |
| Present compound 4-12 + picoxystrobin | 5 + 50 | 100 |
| Present compound 4-12 + picoxystrobin | 10 + 1 | 100 |
| Present compound 4-12 + prothioconazole | 10 + 10 | 100 |
| Present compound 4-12 + prothioconazole | 5 + 50 | 100 |
| Present compound 4-12 + prothioconazole | 30 + 10 | 100 |
| Present compound 4-12 + fenpicoxamid | 10 + 10 | 100 |
| Present compound 4-12 + fenpicoxamid | 5 + 50 | 100 |
| Present compound 4-12 + fenpicoxamid | 30 + 10 | 100 |
| Present compound 4-12 + florylpicoxamid | 10 + 10 | 100 |
| Present compound 4-12 + florylpicoxamid | 5 + 50 | 100 |
| Present compound 4-12 + florylpicoxamid | 30 + 10 | 100 |
| Present compound 4-12 + 3-(2-methylphenyl)butan-2-yl N-[(3-hydroxy-4- | 10 + 10 | 100 |
| methoxypyridin-2-yl)carbonyl]alaninate | | |
| Present compound 4-12 + 3-(2-methylphenyl)butan-2-yl N-[(3-hydroxy-4- | 5 + 50 | 100 |
| methoxypyridin-2-yl)carbonyl]alaninate | | |
| Present compound 4-12 + 3-(2-methylphenyl)butan-2-yl N-[(3-hydroxy-4- | 10 + 1 | 100 |
| methoxypyridin-2-yl)carbonyl]alaninate | | |
| Present compound 4-12 + 3-(2,4-dimethylphenyl)butan-2-yl N-[(3-acetoxy-4- | 10 + 10 | 100 |
| methoxypyridin-2-yl)carbonyl]alaninate | | |
| Present compound 4-12 + 3-(2,4-dimethylphenyl)butan-2-yl N-[(3-acetoxy-4- | 5 + 50 | 100 |
| methoxypyridin-2-yl)carbonyl]alaninate | | |
| Present compound 4-12 + 3-(2,4-dimethylphenyl)butan-2-yl N-[(3-acetoxy-4- | 10 + 1 | 100 |
| methoxypyridin-2-yl)carbonyl]alaninate | | |
| Present compound 4-12 + 3-(4-fluoro-2-methylphenyl)butan-2-yl N-({3-[(2- | 10 + 10 | 100 |
| methylpropanoyl)oxy]-4-methoxypyridin-2- | | |
| yl}carbonyl)alaninate | | |
| Present compound 4-12 + 3-(4-fluoro-2-methylphenyl)butan-2-yl N-({3-[(2- | 5 + 50 | 100 |
| methylpropanoyl)oxy]-4-methoxypyridin-2- | | |
| yl}carbonyl)alaninate | | |
| Present compound 4-12 + 3-(4-fluoro-2-methylphenyl)butan-2-yl N-({3-[(2- | 10 + 1 | 100 |
| methylpropanoyl)oxy]-4-methoxypyridin-2- | | |
| yl}carbonyl)alaninate | | |
| Present compound 4-12 + 3-(4-methoxy-2-methylphenyl)butan-2-yl N-[(3- | 10 + 10 | 100 |
| hydroxy-4-methoxypyridin-2-yl)carbonyl]alaninate | | |
| Present compound 4-12 + 3-(4-methoxy-2-methylphenyl)butan-2-yl N-[(3- | 5 + 50 | 100 |
| hydroxy-4-methoxypyridin-2-yl)carbonyl]alaninate | | |
| Present compound 4-12 + 3-(4-methoxy-2-methylphenyl)butan-2-yl N-[(3- | 10 + 1 | 100 |
| hydroxy-4-methoxypyridin-2-yl)carbonyl]alaninate | | |

TABLE B8

| | | | Conc. | Control |
|---|---|---|---|---|
| Present composition | | | (ppm) | Effect |
| Present | + | benzovindiflupyr | 10 + 10 | 100 |
| compound 4-14 | | | | |
| Present | + | benzovindiflupyr | 3 + 30 | 100 |
| compound 4-14 | | | | |
| Present | + | benzovindiflupyr | 10 + 1 | 100 |
| compound 4-14 | | | | |
| Present | + | 3-(difluoromethyl)-N-(2,3-dihydro-1,1,3- | 10 + 10 | 100 |
| compound 4-14 | | trimethyl-1H-inden-4-yl)-1-methyl-1H-pyrazole-4- | | |
| | | carboxamide | | |
| Present | + | 3-(difluoromethyl)-N-(2,3-dihydro-1,1,3- | 3 + 30 | 100 |
| compound 4-14 | | trimethyl-1H-inden-4-yl)-1-methyl-1H-pyrazole-4- | | |
| | | carboxamide | | |
| Present | + | 3-(difluoromethyl)-N-(2,3-dihydro-1,1,3- | 10 + 1 | 100 |
| compound 4-14 | | trimethyl-1H-inden-4-yl)-1-methyl-1H-pyrazole-4- | | |
| | | carboxamide | | |
| Present | + | fluindapyr | 10 + 10 | 100 |
| compound 4-14 | | | | |
| Present | + | fluindapyr | 3 + 30 | 100 |
| compound 4-14 | | | | |
| Present | + | fluindapyr | 10 + 1 | 100 |
| compound 4-14 | | | | |
| Present | + | fluxapyroxad | 10 + 10 | 100 |
| compound 4-14 | | | | |
| Present | + | fluxapyroxad | 3 + 30 | 100 |
| compound 4-14 | | | | |
| Present | + | fluxapyroxad | 10 + 1 | 100 |
| compound 4-14 | | | | |
| Present | + | Compound of formula (1) | 10 + 10 | 100 |
| compound 4-14 | | | | |
| Present | + | Compound of formula (1) | 3 + 30 | 100 |
| compound 4-14 | | | | |
| Present | + | Compound of formula (1) | 10 + 1 | 100 |
| compound 4-14 | | | | |
| Present | + | Compound of formula (2) | 10 + 10 | 100 |
| compound 4-14 | | | | |
| Present | + | Compound of formula (2) | 3 + 30 | 100 |
| compound 4-14 | | | | |
| Present | + | Compound of formula (2) | 10 + 1 | 100 |
| compound 4-14 | | | | |
| Present | + | Compound of formula (3) | 10 + 10 | 100 |
| compound 4-14 | | | | |
| Present | + | Compound of formula (3) | 3 + 30 | 100 |
| compound 4-14 | | | | |
| Present | + | Compound of formula (3) | 10 + 1 | 100 |
| compound 4-14 | | | | |
| Present | + | pyraclostrobin | 10 + 10 | 100 |
| compound 4-14 | | | | |
| Present | + | pyraclostrobin | 5 + 50 | 100 |
| compound 4-14 | | | | |
| Present | + | pyraclostrobin | 10 + 1 | 100 |
| compound 4-14 | | | | |
| Present | + | picoxystrobin | 10 + 10 | 100 |
| compound 4-14 | | | | |
| Present | + | picoxystrobin | 5 + 50 | 100 |
| compound 4-14 | | | | |
| Present | + | picoxystrobin | 10 + 1 | 100 |
| compound 4-14 | | | | |
| Present | + | prothioconazole | 10 + 10 | 100 |
| compound 4-14 | | | | |
| Present | | prothioconazole | 5 + 50 | 100 |
| compound 4-14 | | | | |
| Present | | prothioconazole | 30 + 10 | 100 |
| compound 4-14 | | | | |
| Present | + | fenpicoxamid | 10 + 10 | 100 |
| compound 4-14 | | | | |
| Present | + | fenpicoxamid | 5 + 50 | 100 |
| compound 4-14 | | | | |
| Present | + | fenpicoxamid | 30 + 10 | 100 |
| compound 4-14 | | | | |
| Present | + | florylpicoxamid | 10 + 10 | 100 |
| compound 4-14 | | | | |
| Present | + | florylpicoxamid | 5 + 50 | 100 |
| compound 4-14 | | | | |
| Present | + | florylpicoxamid | 30 + 10 | 100 |
| compound 4-14 | | | | |
| Present | + | 3-(2-methylpheny])butan-2-yl N-[(3-hydroxy-4- | 10 + 10 | 100 |
| compound 4-14 | | methoxypyridin-2-yl)carbonyl]alaninate | | |
| Present | + | 3-(2-methylpheny])butan-2-yl N-[(3-hydroxy-4- | 5 + 50 | 100 |
| compound 4-14 | | methoxypyridin-2-yl)carbonyl]alaninate | | |
| Present | + | 3-(2-methylpheny])butan-2-yl N-[(3-hydroxy-4- | 10 + 1 | 100 |
| compound 4-14 | | methoxypyridin-2-yl)carbonyl]alaninate | | |
| Present | + | 3-(2,4-dimethylpheny])butan-2-yl N-[(3-acetoxy-4- | 10 + 10 | 100 |
| compound 4-14 | | methoxypyridin-2-yl)carbonyl]alaninate | | |
| Present | + | 3-(2,4-dimethylpheny])butan-2-yl N-[(3-acetoxy-4- | 5 + 50 | 100 |
| compound 4-14 | | methoxypyridin-2-yl)carbonyl]alaninate | | |
| Present | + | 3-(2,4-dimethylpheny])butan-2-yl N-[(3-acetoxy-4- | 10 + 1 | 100 |
| compound 4-14 | | methoxypyridin-2-yl)carbonyl]alaninate | | |
| Present | + | 3-(4-fluoro-2-methylphenyl)butan-2-yl N-({3-[(2- | 10 + 10 | 100 |
| compound 4-14 | | methylpropanoyl)oxy]-4-methoxypyridin-2- | | |
| | | yl}carbonyl)alaninate | | |
| Present | + | 3-(4-fluoro-2-methylpheny])butan-2-yl N-({3-[(2- | 5 + 50 | 100 |
| compound 4-14 | | methylpropanoyl)oxy]-4-methoxypyridin-2- | | |
| | | yl}carbonyl)alaninate | | |
| Present | + | 3-(4-fluoro-2-methylpheny])butan-2-yl N-({3-[(2- | 10 + 1 | 100 |
| compound 4-14 | | methylpropanoyl)oxy]-4-methoxypyridin-2- | | |
| | | yl}carbonyl)alaninate | | |
| Present | + | 3-(4-methoxy-2-methylpheny])butan-2-yl N-[(3- | 10 + 10 | 100 |
| compound 4-14 | | hydroxy-4-methoxypyridin-2-yl)carbonyl]alaninate | | |
| Present | + | 3-(4-methoxy-2-methylpheny])butan-2-yl N-[(3- | 5 + 50 | 100 |
| compound 4-14 | | hydroxy-4-methoxypyridin-2-yl)carbonyl]alaninate | | |
| Present | + | 3-(4-methoxy-2-methylpheny])butan-2-yl N-[(3- | 10 + 1 | 100 |
| compound 4-14 | | hydroxy-4-methoxypyridin-2-yl)carbonyl]alaninate | | |

TABLE B9

| | | | Conc. | Control |
|---|---|---|---|---|
| Present composition | | | (ppm) | Effect |
| Present | + | benzovindiflupyr | 10 + 10 | 100 |
| compound 4-13 | | | | |
| Present | + | benzovindiflupyr | 3 + 30 | 100 |
| compound 4-13 | | | | |
| Present | + | benzovindiflupyr | 10 + 1 | 100 |
| compound 4-13 | | | | |
| Present | + | 3-(difluoromethyl)-N-(2,3-dihydro-1,1,3- | 10 + 10 | 100 |
| compound 4-13 | | trimethyl-1H-inden-4-yl)-1-methyl-1H-pyrazole-4- | | |
| | | carboxamide | | |
| Present | + | 3-(difluoromethyl)-N-(2,3-dihydro-1,1,3- | 3 + 30 | 100 |
| compound 4-13 | | trimethyl-1H-inden-4-yl)-1-methyl-1H-pyrazole-4- | | |
| | | carboxamide | | |
| Present | + | 3-(difluoromethyl)-N-(2,3-dihydro-1,1,3- | 10 + 1 | 100 |
| compound 4-13 | | trimethyl-1H-inden-4-yl)-1-methyl-1H-pyrazole-4- | | |
| | | carboxamide | | |
| Present | + | fluindapyr | 10 + 10 | 100 |
| compound 4-13 | | | | |
| Present | + | fluindapyr | 3 + 30 | 100 |
| compound 4-13 | | | | |
| Present | + | fluindapyr | 10 + 1 | 100 |
| compound 4-13 | | | | |
| Present | + | fluxapyroxad | 10 + 10 | 100 |
| compound 4-13 | | | | |
| Present | + | fluxapyroxad | 3 + 30 | 100 |
| compound 4-13 | | | | |
| Present | + | fluxapyroxad | 10 + 1 | 100 |
| compound 4-13 | | | | |
| Present | + | Compound of formula (1) | 10 + 10 | 100 |
| compound 4-13 | | | | |
| Present | + | Compound of formula (1) | 3 + 30 | 100 |
| compound 4-13 | | | | |
| Present | + | Compound of formula (1) | 10 + 1 | 100 |
| compound 4-13 | | | | |
| Present | + | Compound of formula (2) | 10 + 10 | 100 |
| compound 4-13 | | | | |
| Present | + | Compound of formula (2) | 3 + 30 | 100 |
| compound 4-13 | | | | |
| Present | + | Compound of formula (2) | 10 + 1 | 100 |
| compound 4-13 | | | | |
| Present | + | Compound of formula (3) | 10 + 10 | 100 |
| compound 4-13 | | | | |
| Present | + | Compound of formula (3) | 3 + 30 | 100 |
| compound 4-13 | | | | |
| Present | + | Compound of formula (3) | 10 + 1 | 100 |
| compound 4-13 | | | | |
| Present | + | pyraclostrobin | 10 + 10 | 100 |
| compound 4-13 | | | | |
| Present | + | pyraclostrobin | 5 + 50 | 100 |
| compound 4-13 | | | | |
| Present | + | pyraclostrobin | 10 + 1 | 100 |
| compound 4-13 | | | | |
| Present | + | picoxystrobin | 10 + 10 | 100 |
| compound 4-13 | | | | |
| Present | + | picoxystrobin | 5 + 50 | 100 |
| compound 4-13 | | | | |
| Present | + | picoxystrobin | 10 + 1 | 100 |
| compound 4-13 | | | | |
| Present | + | prothioconazole | 10 + 10 | 100 |
| compound 4-13 | | | | |
| Present | | prothioconazole | 5 + 50 | 100 |
| compound 4-13 | | | | |
| Present | | prothioconazole | 30 + 10 | 100 |
| compound 4-13 | | | | |
| Present | + | fenpicoxamid | 10 + 10 | 100 |
| compound 4-13 | | | | |
| Present | + | fenpicoxamid | 5 + 50 | 100 |
| compound 4-13 | | | | |
| Present | + | fenpicoxamid | 30 + 10 | 100 |
| compound 4-13 | | | | |
| Present | + | florylpicoxamid | 10 + 10 | 100 |
| compound 4-13 | | | | |
| Present | + | florylpicoxamid | 5 + 50 | 100 |
| compound 4-13 | | | | |
| Present | + | florylpicoxamid | 30 + 10 | 100 |
| compound 4-13 | | | | |
| Present | + | 3-(2-methylpheny])butan-2-yl N-[(3-hydroxy-4- | 10 + 10 | 100 |
| compound 4-13 | | methoxypyridin-2-yl)carbonyl]alaninate | | |
| Present | + | 3-(2-methylpheny])butan-2-yl N-[(3-hydroxy-4- | 5 + 50 | 100 |
| compound 4-13 | | methoxypyridin-2-yl)carbonyl]alaninate | | |
| Present | + | 3-(2-methylpheny])butan-2-yl N-[(3-hydroxy-4- | 10 + 1 | 100 |
| compound 4-13 | | methoxypyridin-2-yl)carbonyl]alaninate | | |
| Present | + | 3-(2,4-dimethylpheny])butan-2-yl N-[(3-acetoxy-4- | 10 + 10 | 100 |
| compound 4-13 | | methoxypyridin-2-yl)carbonyl]alaninate | | |
| Present | + | 3-(2,4-dimethylpheny])butan-2-yl N-[(3-acetoxy-4- | 5 + 50 | 100 |
| compound 4-13 | | methoxypyridin-2-yl)carbonyl]alaninate | | |
| Present | + | 3-(2,4-dimethylpheny])butan-2-yl N-[(3-acetoxy-4- | 10 + 1 | 100 |
| compound 4-13 | | methoxypyridin-2-yl)carbonyl]alaninate | | |
| Present | + | 3-(4-fluoro-2-methylphenyl)butan-2-yl N-({3-[(2- | 10 + 10 | 100 |
| compound 4-13 | | methylpropanoyl)oxy]-4-methoxypyridin-2- | | |
| | | yl}carbonyl)alaninate | | |
| Present | + | 3-(4-fluoro-2-methylpheny])butan-2-yl N-({3-[(2- | 5 + 50 | 100 |
| compound 4-13 | | methylpropanoyl)oxy]-4-methoxypyridin-2- | | |
| | | yl}carbonyl)alaninate | | |
| Present | + | 3-(4-fluoro-2-methylphenyl)butan-2-yl N-({3-[(2- | 10 + 1 | 100 |
| compound 4-13 | | methylpropanoyl)oxy]-4-methoxypyridin-2- | | |
| | | yl}carbonyl)alaninate | | |
| Present | + | 3-(4-methoxy-2-methylpheny])butan-2-yl N-[(3- | 10 + 10 | 100 |
| compound 4-13 | | hydroxy-4-methoxypyridin-2-yl)carbonyl]alaninate | | |
| Present | + | 3-(4-methoxy-2-methylpheny])butan-2-yl N-[(3- | 5 + 50 | 100 |
| compound 4-13 | | hydroxy-4-methoxypyridin-2-yl)carbonyl]alaninate | | |
| Present | + | 3-(4-methoxy-2-methylpheny])butan-2-yl N-[(3- | 10 + 1 | 100 |
| compound 4-13 | | hydroxy-4-methoxypyridin-2-yl)carbonyl]alaninate | | |

TABLE B10

| | | | Conc. | Control |
|---|---|---|---|---|
| Present composition | | | (ppm) | Effect |
| Present | + | benzovindiflupyr | 10 + 10 | 100 |
| compound 4-15 | | | | |
| Present | + | benzovindiflupyr | 3 + 30 | 100 |
| compound 4-15 | | | | |
| Present | + | benzovindiflupyr | 10 + 1 | 100 |
| compound 4-15 | | | | |
| Present | + | 3-(difluoromethyl)-N-(2,3-dihydro-1,1,3- | 10 + 10 | 100 |
| compound 4-15 | | trimethyl-1H-inden-4-yl)-1-methyl-1H-pyrazole-4- | | |
| | | carboxamide | | |
| Present | + | 3-(difluoromethyl)-N-(2,3-dihydro-1,1,3- | 3 + 30 | 100 |
| compound 4-15 | | trimethyl-1H-inden-4-yl)-1-methyl-1H-pyrazole-4- | | |
| | | carboxamide | | |
| Present | + | 3-(difluoromethyl)-N-(2,3-dihydro-1,1,3- | 10 + 1 | 100 |
| compound 4-15 | | trimethyl-1H-inden-4-yl)-1-methyl-1H-pyrazole-4- | | |
| | | carboxamide | | |
| Present | + | fluindapyr | 10 + 10 | 100 |
| compound 4-15 | | | | |
| Present | + | fluindapyr | 3 + 30 | 100 |
| compound 4-15 | | | | |
| Present | + | fluindapyr | 10 + 1 | 100 |
| compound 4-15 | | | | |
| Present | + | fluxapyroxad | 10 + 10 | 100 |
| compound 4-15 | | | | |
| Present | + | fluxapyroxad | 3 + 30 | 100 |
| compound 4-15 | | | | |
| Present | + | fluxapyroxad | 10 + 1 | 100 |
| compound 4-15 | | | | |
| Present | + | Compound of formula (1) | 10 + 10 | 100 |
| compound 4-15 | | | | |
| Present | + | Compound of formula (1) | 3 + 30 | 100 |
| compound 4-15 | | | | |
| Present | + | Compound of formula (1) | 10 + 1 | 100 |
| compound 4-15 | | | | |
| Present | + | Compound of formula (2) | 10 + 10 | 100 |
| compound 4-15 | | | | |
| Present | + | Compound of formula (2) | 3 + 30 | 100 |
| compound 4-15 | | | | |
| Present | + | Compound of formula (2) | 10 + 1 | 100 |
| compound 4-15 | | | | |
| Present | + | Compound of formula (3) | 10 + 10 | 100 |
| compound 4-15 | | | | |
| Present | + | Compound of formula (3) | 3 + 30 | 100 |
| compound 4-15 | | | | |
| Present | + | Compound of formula (3) | 10 + 1 | 100 |
| compound 4-15 | | | | |
| Present | + | pyraclostrobin | 10 + 10 | 100 |
| compound 4-15 | | | | |
| Present | + | pyraclostrobin | 5 + 50 | 100 |
| compound 4-15 | | | | |
| Present | + | pyraclostrobin | 10 + 1 | 100 |
| compound 4-15 | | | | |
| Present | + | picoxystrobin | 10 + 10 | 100 |
| compound 4-15 | | | | |
| Present | + | picoxystrobin | 5 + 50 | 100 |
| compound 4-15 | | | | |
| Present | + | picoxystrobin | 10 + 1 | 100 |
| compound 4-15 | | | | |
| Present | + | prothioconazole | 10 + 10 | 100 |
| compound 4-15 | | | | |
| Present | | prothioconazole | 5 + 50 | 100 |
| compound 4-15 | | | | |
| Present | | prothioconazole | 30 + 10 | 100 |
| compound 4-15 | | | | |
| Present | + | fenpicoxamid | 10 + 10 | 100 |
| compound 4-15 | | | | |
| Present | + | fenpicoxamid | 5 + 50 | 100 |
| compound 4-15 | | | | |
| Present | + | fenpicoxamid | 30 + 10 | 100 |
| compound 4-15 | | | | |
| Present | + | florylpicoxamid | 10 + 10 | 100 |
| compound 4-15 | | | | |
| Present | + | florylpicoxamid | 5 + 50 | 100 |
| compound 4-15 | | | | |
| Present | + | florylpicoxamid | 30 + 10 | 100 |
| compound 4-15 | | | | |
| Present | + | 3-(2-methylpheny])butan-2-yl N-[(3-hydroxy-4- | 10 + 10 | 100 |
| compound 4-15 | | methoxypyridin-2-yl)carbonyl]alaninate | | |
| Present | + | 3-(2-methylpheny])butan-2-yl N-[(3-hydroxy-4- | 5 + 50 | 100 |
| compound 4-15 | | methoxypyridin-2-yl)carbonyl]alaninate | | |
| Present | + | 3-(2-methylphenyl)butan-2-yl N-[(3-hydroxy-4- | 10 + 1 | 100 |
| compound 4-15 | | methoxypyridin-2-yl)carbonyl]alaninate | | |
| Present | + | 3-(2,4-dimethylphenyl)butan-2-yl N-[(3-acetoxy-4- | 10 + 10 | 100 |
| compound 4-15 | | methoxypyridin-2-yl)carbonyl]alaninate | | |
| Present | + | 3-(2,4-dimethylphenyl)butan-2-yl N-[(3-acetoxy-4- | 5 + 50 | 100 |
| compound 4-15 | | methoxypyridin-2-yl)carbonyl]alaninate | | |
| Present | + | 3-(2,4-dimethylpheny])butan-2-yl N-[(3-acetoxy-4- | 10 + 1 | 100 |
| compound 4-15 | | methoxypyridin-2-yl)carbonyl]alaninate | | |
| Present | + | 3-(4-fluoro-2-methylphenyl)butan-2-yl N-({3-[(2- | 10 + 10 | 100 |
| compound 4-15 | | methylpropanoyl)oxy]-4-methoxypyridin-2- | | |
| | | yl}carbonyl)alaninate | | |
| Present | + | 3-(4-fluoro-2-methylphenyl)butan-2-yl N-({3-[(2- | 5 + 50 | 100 |
| compound 4-15 | | methylpropanoyl)oxy]-4-methoxypyridin-2- | | |
| | | yl}carbonyl)alaninate | | |
| Present | + | 3-(4-fluoro-2-methylphenyl)butan-2-yl N-({3-[(2- | 10 + 1 | 100 |
| compound 4-15 | | methylpropanoyl)oxy]-4-methoxypyridin-2- | | |
| | | yl}carbonyl)alaninate | | |
| Present | + | 3-(4-methoxy-2-methylpheny])butan-2-yl N-[(3- | 10 + 10 | 100 |
| compound 4-15 | | hydroxy-4-methoxypyridin-2-yl)carbonyl]alaninate | | |
| Present | + | 3-(4-methoxy-2-methylpheny])butan-2-yl N-[(3- | 5 + 50 | 100 |
| compound 4-15 | | hydroxy-4-methoxypyridin-2-yl)carbonyl]alaninate | | |
| Present | + | 3-(4-methoxy-2-methylpheny])butan-2-yl N-[(3- | 10 + 1 | 100 |
| compound 4-15 | | hydroxy-4-methoxypyridin-2-yl)carbonyl]alaninate | | |

TABLE B11

| | | | Conc. | Control |
|---|---|---|---|---|
| Present composition | | | (ppm) | Effect |
| Present | + | benzovindiflupyr | 10 + 10 | 100 |
| compound 14-4 | | | | |
| Present | + | benzovindiflupyr | 3 + 30 | 100 |
| compound 14-4 | | | | |
| Present | + | benzovindiflupyr | 10 + 1 | 100 |
| compound 14-4 | | | | |
| Present | + | 3-(difluoromethyl)-N-(2,3-dihydro-1,1,3- | 10 + 10 | 100 |
| compound 14-4 | | trimethyl-1H-inden-4-yl)-1-methyl-1H-pyrazole-4- | | |
| | | carboxamide | | |
| Present | + | 3-(difluoromethyl)-N-(2,3-dihydro-1,1,3- | 3 + 30 | 100 |
| compound 14-4 | | trimethyl-1H-inden-4-yl)-1-methyl-1H-pyrazole-4- | | |
| | | carboxamide | | |
| Present | + | 3-(difluoromethyl)-N-(2,3-dihydro-1,1,3- | 10 + 1 | 100 |
| compound 14-4 | | trimethyl-1H-inden-4-yl)-1-methyl-1H-pyrazole-4- | | |
| | | carboxamide | | |
| Present | + | fluindapyr | 10 + 10 | 100 |
| compound 14-4 | | | | |
| Present | + | fluindapyr | 3 + 30 | 100 |
| compound 14-4 | | | | |
| Present | + | fluindapyr | 10 + 1 | 100 |
| compound 14-4 | | | | |
| Present | + | fluxapyroxad | 10 + 10 | 100 |
| compound 14-4 | | | | |
| Present | + | fluxapyroxad | 3 + 30 | 100 |
| compound 14-4 | | | | |
| Present | + | fluxapyroxad | 10 + 1 | 100 |
| compound 14-4 | | | | |
| Present | + | Compound of formula (1) | 10 + 10 | 100 |
| compound 14-4 | | | | |
| Present | + | Compound of formula (1) | 3 + 30 | 100 |
| compound 14-4 | | | | |
| Present | + | Compound of formula (1) | 10 + 1 | 100 |
| compound 14-4 | | | | |
| Present | + | Compound of formula (2) | 10 + 10 | 100 |
| compound 14-4 | | | | |
| Present | + | Compound of formula (2) | 3 + 30 | 100 |
| compound 14-4 | | | | |
| Present | + | Compound of formula (2) | 10 + 1 | 100 |
| compound 14-4 | | | | |
| Present | + | Compound of formula (3) | 10 + 10 | 100 |
| compound 14-4 | | | | |
| Present | | Compound of formula (3) | 3 + 30 | 100 |
| compound 14-4 | | | | |
| Present | + | Compound of formula (3) | 10 + 1 | 100 |
| compound 14-4 | | | | |
| Present | + | pyraclostrobin | 10 + 10 | 100 |
| compound 14-4 | | | | |
| Present | + | pyraclostrobin | 5 + 50 | 100 |
| compound 14-4 | | | | |
| Present | + | pyraclostrobin | 10 + 1 | 100 |
| compound 14-4 | | | | |
| Present | + | picoxystrobin | 10 + 10 | 100 |
| compound 14-4 | | | | |
| Present | + | picoxystrobin | 5 + 50 | 100 |
| compound 14-4 | | | | |
| Present | + | picoxystrobin | 10 + 1 | 100 |
| compound 14-4 | | | | |
| Present | + | prothioconazole | 10 + 10 | 100 |
| compound 14-4 | | | | |
| Present | | prothioconazole | 5 + 50 | 100 |
| compound 14-4 | | | | |
| Present | | prothioconazole | 30 + 10 | 100 |
| compound 14-4 | | | | |
| Present | + | fenpicoxamid | 10 + 10 | 100 |
| compound 14-4 | | | | |
| Present | + | fenpicoxamid | 5 + 50 | 100 |
| compound 14-4 | | | | |
| Present | + | fenpicoxamid | 30 + 10 | 100 |
| compound 14-4 | | | | |
| Present | + | florylpicoxamid | 10 + 10 | 100 |
| compound 14-4 | | | | |
| Present | + | florylpicoxamid | 5 + 50 | 100 |
| compound 14-4 | | | | |
| Present | + | florylpicoxamid | 30 + 10 | 100 |
| compound 14-4 | | | | |
| Present | + | 3-(2-methylpheny])butan-2-yl N-[(3-hydroxy-4- | 10 + 10 | 100 |
| compound 14-4 | | methoxypyridin-2-yl)carbonyl]alaninate | | |
| Present | + | 3-(2-methylpheny])butan-2-yl N-[(3-hydroxy-4- | 5 + 50 | 100 |
| compound 14-4 | | methoxypyridin-2-yl)carbonyl]alaninate | | |
| Present | + | 3-(2-methylpheny])butan-2-yl N-[(3-hydroxy-4- | 10 + 1 | 100 |
| compound 14-4 | | methoxypyridin-2-yl)carbonyl]alaninate | | |
| Present | + | 3-(2,4-dimethylpheny])butan-2-yl N-[(3-acetoxy-4- | 10 + 10 | 100 |
| compound 14-4 | | methoxypyridin-2-yl)carbonyl]alaninate | | |
| Present | + | 3-(2,4-dimethylpheny])butan-2-yl N-[(3-acetoxy-4- | 5 + 50 | 100 |
| compound 14-4 | | methoxypyridin-2-yl)carbonyl]alaninate | | |
| Present | + | 3-(2,4-dimethylpheny])butan-2-yl N-[(3-acetoxy-4- | 10 + 1 | 100 |
| compound 14-4 | | methoxypyridin-2-yl)carbonyl]alaninate | | |
| Present | + | 3-(4-fluoro-2-methylphenyl)butan-2-yl N-({3-[(2- | 10 + 10 | 100 |
| compound 14-4 | | methylpropanoyl)oxy]-4-methoxypyridin-2- | | |
| | | yl}carbonyl)alaninate | | |
| Present | + | 3-(4-fluoro-2-methylpheny])butan-2-yl N-({3-[(2- | 5 + 50 | 100 |
| compound 14-4 | | methylpropanoyl)oxy]-4-methoxypyridin-2- | | |
| | | yl}carbonyl)alaninate | | |
| Present | + | 3-(4-fluoro-2-methylphenyl)butan-2-yl N-({3-[(2- | 10 + 1 | 100 |
| compound 14-4 | | methylpropanoyl)oxy]-4-methoxypyridin-2- | | |
| | | yl}carbonyl)alaninate | | |
| Present | + | 3-(4-methoxy-2-methylpheny])butan-2-yl N-[(3- | 10 + 10 | 100 |
| compound 14-4 | | hydroxy-4-methoxypyridin-2-yl)carbonyl]alaninate | | |
| Present | + | 3-(4-methoxy-2-methylpheny])butan-2-yl N-[(3- | 5 + 50 | 100 |
| compound 14-4 | | hydroxy-4-methoxypyridin-2-yl)carbonyl]alaninate | | |
| Present | + | 3-(4-methoxy-2-methylpheny])butan-2-yl N-[(3- | 10 + 1 | 100 |
| compound 14-4 | | hydroxy-4-methoxypyridin-2-yl)carbonyl]alaninate | | |

TABLE B12

| | | | Conc. | Control |
|---|---|---|---|---|
| Present composition | | | (ppm) | Effect |
| Present | + | benzovindiflupyr | 10 + 10 | 100 |
| compound 14-7 | | | | |
| Present | + | benzovindiflupyr | 3 + 30 | 100 |
| compound 14-7 | | | | |
| Present | + | benzovindiflupyr | 10 + 1 | 100 |
| compound 14-7 | | | | |
| Present | + | 3-(difluoromethyl)-N-(2,3-dihydro-1,1,3- | 10 + 10 | 100 |
| compound 14-7 | | trimethyl-1H-inden-4-yl)-1-methyl-1H-pyrazole-4- | | |
| | | carboxamide | | |
| Present | + | 3-(difluoromethyl)-N-(2,3-dihydro-1,1,3- | 3 + 30 | 100 |
| compound 14-7 | | trimethyl-1H-inden-4-yl)-1-methyl-1H-pyrazole-4- | | |
| | | carboxamide | | |
| Present | + | 3-(difluoromethyl)-N-(2,3-dihydro-1,1,3- | 10 + 1 | 100 |
| compound 14-7 | | trimethyl-1H-inden-4-yl)-1-methyl-1H-pyrazole-4- | | |
| | | carboxamide | | |
| Present | + | fluindapyr | 10 + 10 | 100 |
| compound 14-7 | | | | |
| Present | + | fluindapyr | 3 + 30 | 100 |
| compound 14-7 | | | | |
| Present | + | fluindapyr | 10 + 1 | 100 |
| compound 14-7 | | | | |
| Present | + | fluxapyroxad | 10 + 10 | 100 |
| compound 14-7 | | | | |
| Present | + | fluxapyroxad | 3 + 30 | 100 |
| compound 14-7 | | | | |
| Present | + | fluxapyroxad | 10 + 1 | 100 |
| compound 14-7 | | | | |
| Present | + | Compound of formula (1) | 10 + 10 | 100 |
| compound 14-7 | | | | |
| Present | + | Compound of formula (1) | 3 + 30 | 100 |
| compound 14-7 | | | | |
| Present | + | Compound of formula (1) | 10 + 1 | 100 |
| compound 14-7 | | | | |
| Present | + | Compound of formula (2) | 10 + 10 | 100 |
| compound 14-7 | | | | |
| Present | + | Compound of formula (2) | 3 + 30 | 100 |
| compound 14-7 | | | | |
| Present | + | Compound of formula (2) | 10 + 1 | 100 |
| compound 14-7 | | | | |
| Present | + | Compound of formula (3) | 10 + 10 | 100 |
| compound 14-7 | | | | |
| Present | + | Compound of formula (3) | 3 + 30 | 100 |
| compound 14-7 | | | | |
| Present | + | Compound of formula (3) | 10 + 1 | 100 |
| compound 14-7 | | | | |
| Present | + | pyraclostrobin | 10 + 10 | 100 |
| compound 14-7 | | | | |
| Present | + | pyraclostrobin | 5 + 50 | 100 |
| compound 14-7 | | | | |
| Present | + | pyraclostrobin | 10 + 1 | 100 |
| compound 14-7 | | | | |
| Present | + | picoxystrobin | 10 + 10 | 100 |
| compound 14-7 | | | | |
| Present | + | picoxystrobin | 5 + 50 | 100 |
| compound 14-7 | | | | |
| Present | + | picoxystrobin | 10 + 1 | 100 |
| compound 14-7 | | | | |
| Present | + | prothioconazole | 10 + 10 | 100 |
| compound 14-7 | | | | |
| Present | | prothioconazole | 5 + 50 | 100 |
| compound 14-7 | | | | |
| Present | | prothioconazole | 30 + 10 | 100 |
| compound 14-7 | | | | |
| Present | + | fenpicoxamid | 10 + 10 | 100 |
| compound 14-7 | | | | |
| Present | + | fenpicoxamid | 5 + 50 | 100 |
| compound 14-7 | | | | |
| Present | + | fenpicoxamid | 30 + 10 | 100 |
| compound 14-7 | | | | |
| Present | + | florylpicoxamid | 10 + 10 | 100 |
| compound 14-7 | | | | |
| Present | + | florylpicoxamid | 5 + 50 | 100 |
| compound 14-7 | | | | |
| Present | + | florylpicoxamid | 30 + 10 | 100 |
| compound 14-7 | | | | |
| Present | + | 3-(2-methylphenyl)butan-2-yl N-[(3-hydroxy-4- | 10 + 10 | 100 |
| compound 14-7 | | methoxypyridin-2-yl)carbonyl]alaninate | | |
| Present | + | 3-(2-methylpheny])butan-2-yl N-[(3-hydroxy-4- | 5 + 50 | 100 |
| compound 14-7 | | methoxypyridin-2-yl)carbonyl]alaninate | | |
| Present | + | 3-(2-methylpheny])butan-2-yl N-[(3-hydroxy-4- | 10 + 1 | 100 |
| compound 14-7 | | methoxypyridin-2-yl)carbonyl]alaninate | | |
| Present | + | 3-(2,4-dimethylpheny])butan-2-yl N-[(3-acetoxy-4- | 10 + 10 | 100 |
| compound 14-7 | | methoxypyridin-2-yl)carbonyl]alaninate | | |
| Present | + | 3-(2,4-dimethylpheny])butan-2-yl N-[(3-acetoxy-4- | 5 + 50 | 100 |
| compound 14-7 | | methoxypyridin-2-yl)carbonyl]alaninate | | |
| Present | + | 3-(2,4-dimethylpheny])butan-2-yl N-[(3-acetoxy-4- | 10 + 1 | 100 |
| compound 14-7 | | methoxypyridin-2-yl)carbonyl]alaninate | | |
| Present | + | 3-(4-fluoro-2-methylpheny])butan-2-yl N-({3-[(2- | 10 + 10 | 100 |
| compound 14-7 | | methylpropanoyl)oxy]-4-methoxypyridin-2- | | |
| | | yl}carbonyl)alaninate | | |
| Present | + | 3-(4-fluoro-2-methylpheny])butan-2-yl N-({3-[(2- | 5 + 50 | 100 |
| compound 14-7 | | methylpropanoyl)oxy]-4-methoxypyridin-2- | | |
| | | yl}carbonyl)alaninate | | |
| Present | + | 3-(4-fluoro-2-methylpheny])butan-2-yl N-({3-[(2- | 10 + 1 | 100 |
| compound 14-7 | | methylpropanoyl)oxy]-4-methoxypyridin-2- | | |
| | | yl}carbonyl)alaninate | | |
| Present | + | 3-(4-methoxy-2-methylpheny])butan-2-yl N-[(3- | 10 + 10 | 100 |
| compound 14-7 | | hydroxy-4-methoxypyridin-2-yl)carbonyl]alaninate | | |
| Present | + | 3-(4-methoxy-2-methylpheny])butan-2-yl N-[(3- | 5 + 50 | 100 |
| compound 14-7 | | hydroxy-4-methoxypyridin-2-yl)carbonyl]alaninate | | |
| Present | + | 3-(4-methoxy-2-methylpheny])butan-2-yl N-[(3- | 10 + 1 | 100 |
| compound 14-7 | | hydroxy-4-methoxypyridin-2-yl)carbonyl]alaninate | | |

TABLE B13

| | | | Conc. | Control |
|---|---|---|---|---|
| Present composition | | | (ppm) | Effect |
| Present | + | benzovindiflupyr | 10 + 10 | 100 |
| compound 14-6 | | | | |
| Present | + | benzovindiflupyr | 3 + 30 | 100 |
| compound 14-6 | | | | |
| Present | + | benzovindiflupyr | 10 + 1 | 100 |
| compound 14-6 | | | | |
| Present | + | 3-(difluoromethyl)-N-(2,3-dihydro-1,1,3- | 10 + 10 | 100 |
| compound 14-6 | | trimethyl-1H-inden-4-yl)-1-methyl-1H-pyrazole-4- | | |
| | | carboxamide | | |
| Present | + | 3-(difluoromethyl)-N-(2,3-dihydro-1,1,3- | 3 + 30 | 100 |
| compound 14-6 | | trimethyl-1H-inden-4-yl)-1-methyl-1H-pyrazole-4- | | |
| | | carboxamide | | |
| Present | + | 3-(difluoromethyl)-N-(2,3-dihydro-1,1,3- | 10 + 1 | 100 |
| compound 14-6 | | trimethyl-1H-inden-4-yl)-1-methyl-1H-pyrazole-4- | | |
| | | carboxamide | | |
| Present | + | fluindapyr | 10 + 10 | 100 |
| compound 14-6 | | | | |
| Present | + | fluindapyr | 3 + 30 | 100 |
| compound 14-6 | | | | |
| Present | + | fluindapyr | 10 + 1 | 100 |
| compound 14-6 | | | | |
| Present | + | fluxapyroxad | 10 + 10 | 100 |
| compound 14-6 | | | | |
| Present | + | fluxapyroxad | 3 + 30 | 100 |
| compound 14-6 | | | | |
| Present | + | fluxapyroxad | 10 + 1 | 100 |
| compound 14-6 | | | | |
| Present | + | Compound of formula (1) | 10 + 10 | 100 |
| compound 14-6 | | | | |
| Present | + | Compound of formula (1) | 3 + 30 | 100 |
| compound 14-6 | | | | |
| Present | + | Compound of formula (1) | 10 + 1 | 100 |
| compound 14-6 | | | | |
| Present | + | Compound of formula (2) | 10 + 10 | 100 |
| compound 14-6 | | | | |
| Present | + | Compound of formula (2) | 3 + 30 | 100 |
| compound 14-6 | | | | |
| Present | + | Compound of formula (2) | 10 + 1 | 100 |
| compound 14-6 | | | | |
| Present | + | Compound of formula (3) | 10 + 10 | 100 |
| compound 14-6 | | | | |
| Present | + | Compound of formula (3) | 3 + 30 | 100 |
| compound 14-6 | | | | |
| Present | + | Compound of formula (3) | 10 + 1 | 100 |
| compound 14-6 | | | | |
| Present | + | pyraclostrobin | 10 + 10 | 100 |
| compound 14-6 | | | | |
| Present | + | pyraclostrobin | 5 + 50 | 100 |
| compound 14-6 | | | | |
| Present | + | pyraclostrobin | 10 + 1 | 100 |
| compound 14-6 | | | | |
| Present | + | picoxystrobin | 10 + 10 | 100 |
| compound 14-6 | | | | |
| Present | + | picoxystrobin | 5 + 50 | 100 |
| compound 14-6 | | | | |
| Present | + | picoxystrobin | 10 + 1 | 100 |
| compound 14-6 | | | | |
| Present | + | prothioconazole | 10 + 10 | 100 |
| compound 14-6 | | | | |
| Present | | prothioconazole | 5 + 50 | 100 |
| compound 14-6 | | | | |
| Present | | prothioconazole | 30 + 10 | 100 |
| compound 14-6 | | | | |
| Present | + | fenpicoxamid | 10 + 10 | 100 |
| compound 14-6 | | | | |
| Present | + | fenpicoxamid | 5 + 50 | 100 |
| compound 14-6 | | | | |
| Present | + | fenpicoxamid | 30 + 10 | 100 |
| compound 14-6 | | | | |
| Present | + | florylpicoxamid | 10 + 10 | 100 |
| compound 14-6 | | | | |
| Present | + | florylpicoxamid | 5 + 50 | 100 |
| compound 14-6 | | | | |
| Present | + | florylpicoxamid | 30 + 10 | 100 |
| compound 14-6 | | | | |
| Present | + | 3-(2-methylpheny])butan-2-yl N-[(3-hydroxy-4- | 10 + 10 | 100 |
| compound 14-6 | | methoxypyridin-2-yl)carbonyl]alaninate | | |
| Present | + | 3-(2-methylpheny])butan-2-yl N-[(3-hydroxy-4- | 5 + 50 | 100 |
| compound 14-6 | | methoxypyridin-2-yl)carbonyl]alaninate | | |
| Present | + | 3-(2-methylpheny])butan-2-yl N-[(3-hydroxy-4- | 10 + 1 | 100 |
| compound 14-6 | | methoxypyridin-2-yl)carbonyl]alaninate | | |
| Present | + | 3-(2,4-dimethylpheny])butan-2-yl N-[(3-acetoxy-4- | 10 + 10 | 100 |
| compound 14-6 | | methoxypyridin-2-yl)carbonyl]alaninate | | |
| Present | + | 3-(2,4-dimethylpheny])butan-2-yl N-[(3-acetoxy-4- | 5 + 50 | 100 |
| compound 14-6 | | methoxypyridin-2-yl)carbonyl]alaninate | | |
| Present | + | 3-(2,4-dimethylpheny])butan-2-yl N-[(3-acetoxy-4- | 10 + 1 | 100 |
| compound 14-6 | | methoxypyridin-2-yl)carbonyl]alaninate | | |
| Present | + | 3-(4-fluoro-2-methylpheny])butan-2-yl N-({3-[(2- | 10 + 10 | 100 |
| compound 14-6 | | methylpropanoyl)oxy]-4-methoxypyridin-2- | | |
| | | yl}carbonyl)alaninate | | |
| Present | + | 3-(4-fluoro-2-methylpheny])butan-2-yl N-({3-[(2- | 5 + 50 | 100 |
| compound 14-6 | | methylpropanoyl)oxy]-4-methoxypyridin-2- | | |
| | | yl}carbonyl)alaninate | | |
| Present | + | 3-(4-fluoro-2-methylpheny])butan-2-yl N-({3-[(2- | 10 + 1 | 100 |
| compound 14-6 | | methylpropanoyl)oxy]-4-methoxypyridin-2- | | |
| | | yl}carbonyl)alaninate | | |
| Present | + | 3-(4-methoxy-2-methylpheny])butan-2-yl N-[(3- | 10 + 10 | 100 |
| compound 14-6 | | hydroxy-4-methoxypyridin-2-yl)carbonyl]alaninate | | |
| Present | + | 3-(4-methoxy-2-methylpheny])butan-2-yl N-[(3- | 5 + 50 | 100 |
| compound 14-6 | | hydroxy-4-methoxypyridin-2-yl)carbonyl]alaninate | | |
| Present | + | 3-(4-methoxy-2-methylpheny])butan-2-yl N-[(3- | 10 + 1 | 100 |
| compound 14-6 | | hydroxy-4-methoxypyridin-2-yl)carbonyl]alaninate | | |

TABLE B14

| | | | Conc. | Control |
|---|---|---|---|---|
| Present composition | | | (ppm) | Effect |
| Present | + | benzovindiflupyr | 10 + 10 | 100 |
| compound 14-2 | | | | |
| Present | + | benzovindiflupyr | 3 + 30 | 100 |
| compound 14-2 | | | | |
| Present | + | benzovindiflupyr | 10 + 1 | 100 |
| compound 14-2 | | | | |
| Present | + | 3-(difluoromethyl)-N-(2,3-dihydro-1,1,3- | 10 + 10 | 100 |
| compound 14-2 | | trimethyl-1H-inden-4-yl)-1-methyl-1H-pyrazole-4- | | |
| | | carboxamide | | |
| Present | + | 3-(difluoromethyl)-N-(2,3-dihydro-1,1,3- | 3 + 30 | 100 |
| compound 14-2 | | trimethyl-1H-inden-4-yl)-1-methyl-1H-pyrazole-4- | | |
| | | carboxamide | | |
| Present | + | 3-(difluoromethyl)-N-(2,3-dihydro-1,1,3- | 10 + 1 | 100 |
| compound 14-2 | | trimethyl-1H-inden-4-yl)-1-methyl-1H-pyrazole-4- | | |
| | | carboxamide | | |
| Present | + | fluindapyr | 10 + 10 | 100 |
| compound 14-2 | | | | |
| Present | + | fluindapyr | 3 + 30 | 100 |
| compound 14-2 | | | | |
| Present | + | fluindapyr | 10 + 1 | 100 |
| compound 14-2 | | | | |
| Present | + | fluxapyroxad | 10 + 10 | 100 |
| compound 14-2 | | | | |
| Present | + | fluxapyroxad | 3 + 30 | 100 |
| compound 14-2 | | | | |
| Present | + | fluxapyroxad | 10 + 1 | 100 |
| compound 14-2 | | | | |
| Present | + | Compound of formula (1) | 10 + 10 | 100 |
| compound 14-2 | | | | |
| Present | + | Compound of formula (1) | 3 + 30 | 100 |
| compound 14-2 | | | | |
| Present | + | Compound of formula (1) | 10 + 1 | 100 |
| compound 14-2 | | | | |
| Present | + | Compound of formula (2) | 10 + 10 | 100 |
| compound 14-2 | | | | |
| Present | + | Compound of formula (2) | 3 + 30 | 100 |
| compound 14-2 | | | | |
| Present | + | Compound of formula (2) | 10 + 1 | 100 |
| compound 14-2 | | | | |
| Present | + | Compound of formula (3) | 10 + 10 | 100 |
| compound 14-2 | | | | |
| Present | + | Compound of formula (3) | 3 + 30 | 100 |
| compound 14-2 | | | | |
| Present | + | Compound of formula (3) | 10 + 1 | 100 |
| compound 14-2 | | | | |
| Present | + | pyraclostrobin | 10 + 10 | 100 |
| compound 14-2 | | | | |
| Present | + | pyraclostrobin | 5 + 50 | 100 |
| compound 14-2 | | | | |
| Present | + | pyraclostrobin | 10 + 1 | 100 |
| compound 14-2 | | | | |
| Present | + | picoxystrobin | 10 + 10 | 100 |
| compound 14-2 | | | | |
| Present | + | picoxystrobin | 5 + 50 | 100 |
| compound 14-2 | | | | |
| Present | + | picoxystrobin | 10 + 1 | 100 |
| compound 14-2 | | | | |
| Present | + | prothioconazole | 10 + 10 | 100 |
| compound 14-2 | | | | |
| Present | | prothioconazole | 5 + 50 | 100 |
| compound 14-2 | | | | |
| Present | | prothioconazole | 30 + 10 | 100 |
| compound 14-2 | | | | |
| Present | + | fenpicoxamid | 10 + 10 | 100 |
| compound 14-2 | | | | |
| Present | + | fenpicoxamid | 5 + 50 | 100 |
| compound 14-2 | | | | |
| Present | + | fenpicoxamid | 30 + 10 | 100 |
| compound 14-2 | | | | |
| Present | + | florylpicoxamid | 10 + 10 | 100 |
| compound 14-2 | | | | |
| Present | + | florylpicoxamid | 5 + 50 | 100 |
| compound 14-2 | | | | |
| Present | + | florylpicoxamid | 30 + 10 | 100 |
| compound 14-2 | | | | |
| Present | + | 3-(2-methylpheny])butan-2-yl N-[(3-hydroxy-4- | 10 + 10 | 100 |
| compound 14-2 | | methoxypyridin-2-yl)carbonyl]alaninate | | |
| Present | + | 3-(2-methylpheny])butan-2-yl N-[(3-hydroxy-4- | 5 + 50 | 100 |
| compound 14-2 | | methoxypyridin-2-yl)carbonyl]alaninate | | |
| Present | + | 3-(2-methylpheny])butan-2-yl N-[(3-hydroxy-4- | 10 + 1 | 100 |
| compound 14-2 | | methoxypyridin-2-yl)carbonyl]alaninate | | |
| Present | + | 3-(2,4-dimethylpheny])butan-2-yl N-[(3-acetoxy-4- | 10 + 10 | 100 |
| compound 14-2 | | methoxypyridin-2-yl)carbonyl]alaninate | | |
| Present | + | 3-(2,4-dimethylpheny])butan-2-yl N-[(3-acetoxy-4- | 5 + 50 | 100 |
| compound 14-2 | | methoxypyridin-2-yl)carbonyl]alaninate | | |
| Present | + | 3-(2,4-dimethylpheny])butan-2-yl N-[(3-acetoxy-4- | 10 + 1 | 100 |
| compound 14-2 | | methoxypyridin-2-yl)carbonyl]alaninate | | |
| Present | + | 3-(4-fluoro-2-methylphenyl)butan-2-yl N-({3-[(2- | 10 + 10 | 100 |
| compound 14-2 | | methylpropanoyl)oxy]-4-methoxypyridin-2- | | |
| | | yl}carbonyl)alaninate | | |
| Present | + | 3-(4-fluoro-2-methylphenyl)butan-2-yl N-({3-[(2- | 5 + 50 | 100 |
| compound 14-2 | | methylpropanoyl)oxy]-4-methoxypyridin-2- | | |
| | | yl}carbonyl)alaninate | | |
| Present | + | 3-(4-fluoro-2-methylphenyl)butan-2-yl N-({3-[(2- | 10 + 1 | 100 |
| compound 14-2 | | methylpropanoyl)oxy]-4-methoxypyridin-2- | | |
| | | yl}carbonyl)alaninate | | |
| Present | + | 3-(4-methoxy-2-methylpheny])butan-2-yl N-[(3- | 10 + 10 | 100 |
| compound 14-2 | | hydroxy-4-methoxypyridin-2-yl)carbonyl]alaninate | | |
| Present | + | 3-(4-methoxy-2-methylpheny])butan-2-yl N-[(3- | 5 + 50 | 100 |
| compound 14-2 | | hydroxy-4-methoxypyridin-2-yl)carbonyl]alaninate | | |
| Present | + | 3-(4-methoxy-2-methylpheny])butan-2-yl N-[(3- | 10 + 1 | 100 |
| compound 14-2 | | hydroxy-4-methoxypyridin-2-yl)carbonyl]alaninate | | |

TABLE B15

| | | | Conc. | Control |
|---|---|---|---|---|
| Present composition | | | (ppm) | Effect |
| Present | + | benzovindiflupyr | 10 + 10 | 100 |
| compound 14-74 | | | | |
| Present | + | benzovindiflupyr | 3 + 30 | 100 |
| compound 14-74 | | | | |
| Present | + | benzovindiflupyr | 10 + 1 | 100 |
| compound 14-74 | | | | |
| Present | + | 3-(difluoromethyl)-N-(2,3-dihydro-1,1,3- | 10 + 10 | 100 |
| compound 14-74 | | trimethyl-1H-inden-4-yl)-1-methyl-1H-pyrazole-4- | | |
| | | carboxamide | | |
| Present | + | 3-(difluoromethyl)-N-(2,3-dihydro-1,1,3- | 3 + 30 | 100 |
| compound 14-74 | | trimethyl-1H-inden-4-yl)-1-methyl-1H-pyrazole-4- | | |
| | | carboxamide | | |
| Present | + | 3-(difluoromethyl)-N-(2,3-dihydro-1,1,3- | 10 + 1 | 100 |
| compound 14-74 | | trimethyl-1H-inden-4-yl)-1-methyl-1H-pyrazole-4- | | |
| | | carboxamide | | |
| Present | | fluindapyr | 10 + 10 | 100 |
| compound 14-74 | | | | |
| Present | + | fluindapyr | 3 + 30 | 100 |
| compound 14-74 | | | | |
| Present | | fluindapyr | 10 + 1 | 100 |
| compound 14-74 | | | | |
| Present | t | fluxapyroxad | 10 + 10 | 100 |
| compound 14-74 | | | | |
| Present | | fluxapyroxad | 3 + 30 | 100 |
| compound 14-74 | | | | |
| Present | + | fluxapyroxad | 10 + 1 | 100 |
| compound 14-74 | | | | |
| Present | + | Compound of formula (1) | 10 + 10 | 100 |
| compound 14-74 | | | | |
| Present | + | Compound of formula (1) | 3 + 30 | 100 |
| compound 14-74 | | | | |
| Present | + | Compound of formula (1) | 10 + 1 | 100 |
| compound 14-74 | | | | |
| Present | + | Compound of formula (2) | 10 + 10 | 100 |
| compound 14-74 | | | | |
| Present | + | Compound of formula (2) | 3 + 30 | 100 |
| compound 14-74 | | | | |
| Present | + | Compound of formula (2) | 10 + 1 | 100 |
| compound 14-74 | | | | |
| Present | + | Compound of formula (3) | 10 + 10 | 100 |
| compound 14-74 | | | | |
| Present | | Compound of formula (3) | 3 + 30 | 100 |
| compound 14-74 | | | | |
| Present | | Compound of formula (3) | 10 + 1 | 100 |
| compound 14-74 | | | | |
| Present | | pyraclostrobin | 10 + 10 | 100 |
| compound 14-74 | | | | |
| Present | + | pyraclostrobin | 5 + 50 | 100 |
| compound 14-74 | | | | |
| Present | + | pyraclostrobin | 10 + 1 | 100 |
| compound 14-74 | | | | |
| Present | | picoxystrobin | 10 + 10 | 100 |
| compound 14-74 | | | | |
| Present | + | picoxystrobin | 5 + 50 | 100 |
| compound 14-74 | | | | |
| Present | + | picoxystrobin | 10 + 1 | 100 |
| compound 14-74 | | | | |
| Present | + | prothioconazole | 10 + 10 | 100 |
| compound 14-74 | | | | |
| Present | | prothioconazole | 5 + 50 | 100 |
| compound 14-74 | | | | |
| Present | | prothioconazole | 30 + 10 | 100 |
| compound 14-74 | | | | |
| Present | + | fenpicoxamid | 10 + 10 | 100 |
| compound 14-74 | | | | |
| Present | + | fenpicoxamid | 5 + 50 | 100 |
| compound 14-74 | | | | |
| Present | + | fenpicoxamid | 30 + 10 | 100 |
| compound 14-74 | | | | |
| Present | + | florylpicoxamid | 10 + 10 | 100 |
| compound 14-74 | | | | |
| Present | + | florylpicoxamid | 5 + 50 | 100 |
| compound 14-74 | | | | |
| Present | + | florylpicoxamid | 30 + 10 | 100 |
| compound 14-74 | | | | |
| Present | + | 3-(2-methylpheny])butan-2-yl N-[(3-hydroxy-4- | 10 + 10 | 100 |
| compound 14-74 | | methoxypyridin-2-yl)carbonyl]alaninate | | |
| Present | + | 3-(2-methylpheny])butan-2-yl N-[(3-hydroxy-4- | 5 + 50 | 100 |
| compound 14-74 | | methoxypyridin-2-yl)carbonyl]alaninate | | |
| Present | + | 3-(2-methylpheny])butan-2-yl N-[(3-hydroxy-4- | 10 + 1 | 100 |
| compound 14-74 | | methoxypyridin-2-yl)carbonyl]alaninate | | |
| Present | + | 3-(2,4-dimethylpheny])butan-2-yl N-[(3-acetoxy-4- | 10 + 10 | 100 |
| compound 14-74 | | methoxypyridin-2-yl)carbonyl]alaninate | | |
| Present | + | 3-(2,4-dimethylpheny])butan-2-yl N-[(3-acetoxy-4- | 5 + 50 | 100 |
| compound 14-74 | | methoxypyridin-2-yl)carbonyl]alaninate | | |
| Present | + | 3-(2,4-dimethylpheny])butan-2-yl N-[(3-acetoxy-4- | 10 + 1 | 100 |
| compound 14-74 | | methoxypyridin-2-yl)carbonyl]alaninate | | |
| Present | + | 3-(4-fluoro-2-methylphenyl)butan-2-yl N-({3-[(2- | 10 + 10 | 100 |
| compound 14-74 | | methylpropanoyl)oxy]-4-methoxypyridin-2- | | |
| | | yl}carbonyl)alaninate | | |
| Present | + | 3-(4-fluoro-2-methylphenyl)butan-2-yl N-({3-[(2- | 5 + 50 | 100 |
| compound 14-74 | | methylpropanoyl)oxy]-4-methoxypyridin-2- | | |
| | | yl}carbonyl)alaninate | | |
| Present | + | 3-(4-fluoro-2-methylpheny])butan-2-yl N-({3-[(2- | 10 + 1 | 100 |
| compound 14-74 | | methylpropanoyl)oxy]-4-methoxypyridin-2- | | |
| | | yl}carbonyl)alaninate | | |
| Present | + | 3-(4-methoxy-2-methylpheny])butan-2-yl N-[(3- | 10 + 10 | 100 |
| compound 14-74 | | hydroxy-4-methoxypyridin-2-yl)carbonyl]alaninate | | |
| Present | + | 3-(4-methoxy-2-methylpheny])butan-2-yl N-[(3- | 5 + 50 | 100 |
| compound 14-74 | | hydroxy-4-methoxypyridin-2-yl)carbonyl]alaninate | | |
| Present | + | 3-(4-methoxy-2-methylpheny])butan-2-yl N-[(3- | 10 + 1 | 100 |
| compound 14-74 | | hydroxy-4-methoxypyridin-2-yl)carbonyl]alaninate | | |

TABLE B16

| | | | Conc. | Control |
|---|---|---|---|---|
| Present composition | | | (ppm) | Effect |
| Present | + | benzovindiflupyr | 10 + 10 | 100 |
| compound 14-10 | | | | |
| Present | + | benzovindiflupyr | 3 + 30 | 100 |
| compound 14-10 | | | | |
| Present | + | benzovindiflupyr | 10 + 1 | 100 |
| compound 14-10 | | | | |
| Present | + | 3-(difluoromethyl)-N-(2,3-dihydro-1,1,3- | 10 + 10 | 100 |
| compound 14-10 | | trimethyl-1H-inden-4-yl)-1-methyl-1H-pyrazole-4- | | |
| | | carboxamide | | |
| Present | + | 3-(difluoromethyl)-N-(2,3-dihydro-1,1,3- | 3 + 30 | 100 |
| compound 14-10 | | trimethyl-1H-inden-4-yl)-1-methyl-1H-pyrazole-4- | | |
| | | carboxamide | | |
| Present | + | 3-(difluoromethyl)-N-(2,3-dihydro-1,1,3- | 10 + 1 | 100 |
| compound 14-10 | | trimethyl-1H-inden-4-yl)-1-methyl-1H-pyrazole-4- | | |
| | | carboxamide | | |
| Present | + | fluindapyr | 10 + 10 | 100 |
| compound 14-10 | | | | |
| Present | + | fluindapyr | 3 + 30 | 100 |
| compound 14-10 | | | | |
| Present | + | fluindapyr | 10 + 1 | 100 |
| compound 14-10 | | | | |
| Present | + | fluxapyroxad | 10 + 10 | 100 |
| compound 14-10 | | | | |
| Present | + | fluxapyroxad | 3 + 30 | 100 |
| compound 14-10 | | | | |
| Present | + | fluxapyroxad | 10 + 1 | 100 |
| compound 14-10 | | | | |
| Present | + | Compound of formula (1) | 10 + 10 | 100 |
| compound 14-10 | | | | |
| Present | + | Compound of formula (1) | 3 + 30 | 100 |
| compound 14-10 | | | | |
| Present | + | Compound of formula (1) | 10 + 1 | 100 |
| compound 14-10 | | | | |
| Present | + | Compound of formula (2) | 10 + 10 | 100 |
| compound 14-10 | | | | |
| Present | + | Compound of formula (2) | 3 + 30 | 100 |
| compound 14-10 | | | | |
| Present | + | Compound of formula (2) | 10 + 1 | 100 |
| compound 14-10 | | | | |
| Present | + | Compound of formula (3) | 10 + 10 | 100 |
| compound 14-10 | | | | |
| Present | + | Compound of formula (3) | 3 + 30 | 100 |
| compound 14-10 | | | | |
| Present | + | Compound of formula (3) | 10 + 1 | 100 |
| compound 14-10 | | | | |
| Present | + | pyraclostrobin | 10 + 10 | 100 |
| compound 14-10 | | | | |
| Present | + | pyraclostrobin | 5 + 50 | 100 |
| compound 14-10 | | | | |
| Present | + | pyraclostrobin | 10 + 1 | 100 |
| compound 14-10 | | | | |
| Present | + | picoxystrobin | 10 + 10 | 100 |
| compound 14-10 | | | | |
| Present | + | picoxystrobin | 5 + 50 | 100 |
| compound 14-10 | | | | |
| Present | + | picoxystrobin | 10 + 1 | 100 |
| compound 14-10 | | | | |
| Present | + | prothioconazole | 10 + 10 | 100 |
| compound 14-10 | | | | |
| Present | | prothioconazole | 5 + 50 | 100 |
| compound 14-10 | | | | |
| Present | | prothioconazole | 30 + 10 | 100 |
| compound 14-10 | | | | |
| Present | + | fenpicoxamid | 10 + 10 | 100 |
| compound 14-10 | | | | |
| Present | + | fenpicoxamid | 5 + 50 | 100 |
| compound 14-10 | | | | |
| Present | + | fenpicoxamid | 30 + 10 | 100 |
| compound 14-10 | | | | |
| Present | + | florylpicoxamid | 10 + 10 | 100 |
| compound 14-10 | | | | |
| Present | + | florylpicoxamid | 5 + 50 | 100 |
| compound 14-10 | | | | |
| Present | + | florylpicoxamid | 30 + 10 | 100 |
| compound 14-10 | | | | |
| Present | + | 3-(2-methylpheny])butan-2-yl N-[(3-hydroxy-4- | 10 + 10 | 100 |
| compound 14-10 | | methoxypyridin-2-yl)carbonyl]alaninate | | |
| Present | + | 3-(2-methylpheny])butan-2-yl N-[(3-hydroxy-4- | 5 + 50 | 100 |
| compound 14-10 | | methoxypyridin-2-yl)carbonyl]alaninate | | |
| Present | + | 3-(2-methylpheny])butan-2-yl N-[(3-hydroxy-4- | 10 + 1 | 100 |
| compound 14-10 | | methoxypyridin-2-yl)carbonyl]alaninate | | |
| Present | + | 3-(2,4-dimethylpheny])butan-2-yl N-[(3-acetoxy-4- | 10 + 10 | 100 |
| compound 14-10 | | methoxypyridin-2-yl)carbonyl]alaninate | | |
| Present | + | 3-(2,4-dimethylpheny])butan-2-yl N-[(3-acetoxy-4- | 5 + 50 | 100 |
| compound 14-10 | | methoxypyridin-2-yl)carbonyl]alaninate | | |
| Present | + | 3-(2,4-dimethylpheny])butan-2-yl N-[(3-acetoxy-4- | 10 + 1 | 100 |
| compound 14-10 | | methoxypyridin-2-yl)carbonyl]alaninate | | |
| Present | + | 3-(4-fluoro-2-methylpheny])butan-2-yl N-({3-[(2- | 10 + 10 | 100 |
| compound 14-10 | | methylpropanoyl)oxy]-4-methoxypyridin-2- | | |
| | | yl}carbonyl)alaninate | | |
| Present | + | 3-(4-fluoro-2-methylphenyl)butan-2-yl N-({3-[(2- | 5 + 50 | 100 |
| compound 14-10 | | methylpropanoyl)oxy]-4-methoxypyridin-2- | | |
| | | yl}carbonyl)alaninate | | |
| Present | + | 3-(4-fluoro-2-methylphenyl)butan-2-yl N-({3-[(2- | 10 + 1 | 100 |
| compound 14-10 | | methylpropanoyl)oxy]-4-methoxypyridin-2- | | |
| | | yl}carbonyl)alaninate | | |
| Present | | 3-(4-methoxy-2-methylpheny])butan-2-yl N-[(3- | 10 + 10 | 100 |
| compound 14-10 | | hydroxy-4-methoxypyridin-2-yl)carbonyl]alaninate | | |
| Present | | 3-(4-methoxy-2-methylpheny])butan-2-yl N-[(3- | 5 + 50 | 100 |
| compound 14-10 | | hydroxy-4-methoxypyridin-2-yl)carbonyl]alaninate | | |
| Present | + | 3-(4-methoxy-2-methylpheny])butan-2-yl N-[(3- | 10 + 1 | 100 |
| compound 14-10 | | hydroxy-4-methoxypyridin-2-yl)carbonyl]alaninate | | |

TABLE B17

| | | | Conc. | Control |
|---|---|---|---|---|
| Present composition | | | (ppm) | Effect |
| Present | + | benzovindiflupyr | 10 + 10 | 100 |
| compound 14-9 | | | | |
| Present | + | benzovindiflupyr | 3 + 30 | 100 |
| compound 14-9 | | | | |
| Present | + | benzovindiflupyr | 10 + 1 | 100 |
| compound 14-9 | | | | |
| Present | + | 3-(difluoromethyl)-N-(2,3-dihydro-1,1,3- | 10 + 10 | 100 |
| compound 14-9 | | trimethyl-1H-inden-4-yl)-1-methyl-1H-pyrazole-4- | | |
| | | carboxamide | | |
| Present | + | 3-(difluoromethyl)-N-(2,3-dihydro-1,1,3- | 3 + 30 | 100 |
| compound 14-9 | | trimethyl-1H-inden-4-yl)-1-methyl-1H-pyrazole-4- | | |
| | | carboxamide | | |
| Present | + | 3-(difluoromethyl)-N-(2,3-dihydro-1,1,3- | 10 + 1 | 100 |
| compound 14-9 | | trimethyl-1H-inden-4-yl)-1-methyl-1H-pyrazole-4- | | |
| | | carboxamide | | |
| Present | + | fluindapyr | 10 + 10 | 100 |
| compound 14-9 | | | | |
| Present | + | fluindapyr | 3 + 30 | 100 |
| compound 14-9 | | | | |
| Present | + | fluindapyr | 10 + 1 | 100 |
| compound 14-9 | | | | |
| Present | + | fluxapyroxad | 10 + 10 | 100 |
| compound 14-9 | | | | |
| Present | + | fluxapyroxad | 3 + 30 | 100 |
| compound 14-9 | | | | |
| Present | + | fluxapyroxad | 10 + 1 | 100 |
| compound 14-9 | | | | |
| Present | + | Compound of formula (1) | 10 + 10 | 100 |
| compound 14-9 | | | | |
| Present | + | Compound of formula (1) | 3 + 30 | 100 |
| compound 14-9 | | | | |
| Present | + | Compound of formula (1) | 10 + 1 | 100 |
| compound 14-9 | | | | |
| Present | + | Compound of formula (2) | 10 + 10 | 100 |
| compound 14-9 | | | | |
| Present | + | Compound of formula (2) | 3 + 30 | 100 |
| compound 14-9 | | | | |
| Present | + | Compound of formula (2) | 10 + 1 | 100 |
| compound 14-9 | | | | |
| Present | + | Compound of formula (3) | 10 + 10 | 100 |
| compound 14-9 | | | | |
| Present | + | Compound of formula (3) | 3 + 30 | 100 |
| compound 14-9 | | | | |
| Present | | Compound of formula (3) | 10 + 1 | 100 |
| compound 14-9 | | | | |
| Present | + | pyraclostrobin | 10 + 10 | 100 |
| compound 14-9 | | | | |
| Present | + | pyraclostrobin | 5 + 50 | 100 |
| compound 14-9 | | | | |
| Present | + | pyraclostrobin | 10 + 1 | 100 |
| compound 14-9 | | | | |
| Present | + | picoxystrobin | 10 + 10 | 100 |
| compound 14-9 | | | | |
| Present | + | picoxystrobin | 5 + 50 | 100 |
| compound 14-9 | | | | |
| Present | + | picoxystrobin | 10 + 1 | 100 |
| compound 14-9 | | | | |
| Present | + | prothioconazole | 10 + 10 | 100 |
| compound 14-9 | | | | |
| Present | | prothioconazole | 5 + 50 | 100 |
| compound 14-9 | | | | |
| Present | | prothioconazole | 30 + 10 | 100 |
| compound 14-9 | | | | |
| Present | + | fenpicoxamid | 10 + 10 | 100 |
| compound 14-9 | | | | |
| Present | + | fenpicoxamid | 5 + 50 | 100 |
| compound 14-9 | | | | |
| Present | + | fenpicoxamid | 30 + 10 | 100 |
| compound 14-9 | | | | |
| Present | + | florylpicoxamid | 10 + 10 | 100 |
| compound 14-9 | | | | |
| Present | + | florylpicoxamid | 5 + 50 | 100 |
| compound 14-9 | | | | |
| Present | + | florylpicoxamid | 30 + 10 | 100 |
| compound 14-9 | | | | |
| Present | + | 3-(2-methylpheny])butan-2-yl N-[(3-hydroxy-4- | 10 + 10 | 100 |
| compound 14-9 | | methoxypyridin-2-yl)carbonyl]alaninate | | |
| Present | | 3-(2-methylpheny])butan-2-yl N-[(3-hydroxy-4- | 5 + 50 | 100 |
| compound 14-9 | | methoxypyridin-2-yl)carbonyl]alaninate | | |
| Present | + | 3-(2-methylpheny])butan-2-yl N-[(3-hydroxy-4- | 10 + 1 | 100 |
| compound 14-9 | | methoxypyridin-2-yl)carbonyl]alaninate | | |
| Present | + | 3-(2,4-dimethylpheny])butan-2-yl N-[(3-acetoxy-4- | 10 + 10 | 100 |
| compound 14-9 | | methoxypyridin-2-yl)carbonyl]alaninate | | |
| Present | + | 3-(2,4-dimethylpheny])butan-2-yl N-[(3-acetoxy-4- | 5 + 50 | 100 |
| compound 14-9 | | methoxypyridin-2-yl)carbonyl]alaninate | | |
| Present | + | 3-(2,4-dimethylpheny])butan-2-yl N-[(3-acetoxy-4- | 10 + 1 | 100 |
| compound 14-9 | | methoxypyridin-2-yl)carbonyl]alaninate | | |
| Present | | 3-(4-fluoro-2-methylpheny])butan-2-yl N-({3-[(2- | 10 + 10 | 100 |
| compound 14-9 | | methylpropanoyl)oxy]-4-methoxypyridin-2- | | |
| | | yl}carbonyl)alaninate | | |
| Present | | 3-(4-fluoro-2-methylphenyl)butan-2-yl N-({3-[(2- | 5 + 50 | 100 |
| compound 14-9 | | methylpropanoyl)oxy]-4-methoxypyridin-2- | | |
| | | yl}carbonyl)alaninate | | |
| Present | + | 3-(4-fluoro-2-methylphenyl)butan-2-yl N-({3-[(2- | 10 + 1 | 100 |
| compound 14-9 | | methylpropanoyl)oxy]-4-methoxypyridin-2- | | |
| | | yl}carbonyl)alaninate | | |
| Present | + | 3-(4-methoxy-2-methylpheny])butan-2-yl N-[(3- | 10 + 10 | 100 |
| compound 14-9 | | hydroxy-4-methoxypyridin-2-yl)carbonyl]alaninate | | |
| Present | | 3-(4-methoxy-2-methylpheny])butan-2-yl N-[(3- | 5 + 50 | 100 |
| compound 14-9 | | hydroxy-4-methoxypyridin-2-yl)carbonyl]alaninate | | |
| Present | + | 3-(4-methoxy-2-methylpheny])butan-2-yl N-[(3- | 10 + 1 | 100 |
| compound 14-9 | | hydroxy-4-methoxypyridin-2-yl)carbonyl]alaninate | | |

TABLE B18

| | Conc. | Control |
|---|---|---|
| Present composition | (ppm) | Effect |
| Present compound 14-14 + benzovindiflupyr | 10 + 10 | 100 |
| Present compound 14-14 + benzovindiflupyr | 3 + 30 | 100 |
| Present compound 14-14 + benzovindiflupyr | 10 + 1 | 100 |
| Present compound 14-14 + 3-(difluoromethyl)-N-(2,3-dihydro-1,1,3- | 10 + 10 | 100 |
| trimethyl-1H-inden-4-yl)-1-methyl-1H-pyrazole-4- | | |
| carboxamide | | |
| Present compound 14-14 + 3-(difluoromethyl)-N-(2,3-dihydro-1,1,3- | 3 + 30 | 100 |
| trimethyl-1H-inden-4-yl)-1-methyl-1H-pyrazole-4- | | |
| carboxamide | | |
| Present compound 14-14 + 3-(difluoromethyl)-N-(2,3-dihydro-1,1,3- | 10 + 1 | 100 |
| trimethyl-1H-inden-4-yl)-1-methyl-1H-pyrazole-4- | | |
| carboxamide | | |
| Present compound 14-14 + fluindapyr | 10 + 10 | 100 |
| Present compound 14-14 + fluindapyr | 3 + 30 | 100 |
| Present compound 14-14 + fluindapyr | 10 + 1 | 100 |
| Present compound 14-14 + fluxapyroxad | 10 + 10 | 100 |
| Present compound 14-14 + fluxapyroxad | 3 + 30 | 100 |
| Present compound 14-14 + fluxapyroxad | 10 + 1 | 100 |
| Present compound 14-14 + Compound of formula (1) | 10 + 10 | 100 |
| Present compound 14-14 + Compound of formula (1) | 3 + 30 | 100 |
| Present compound 14-14 + Compound of formula (1) | 10 + 1 | 100 |
| Present compound 14-14 + Compound of formula (2) | 10 + 10 | 100 |
| Present compound 14-14 + Compound of formula (2) | 3 + 30 | 100 |
| Present compound 14-14 + Compound of formula (2) | 10 + 1 | 100 |
| Present compound 14-14 + Compound of formula (3) | 10 + 10 | 100 |
| Present compound 14-14 + Compound of formula (3) | 3 + 30 | 100 |
| Present compound 14-14 + Compound of formula (3) | 10 + 1 | 100 |
| Present compound 14-14 + pyraclostrobin | 10 + 10 | 100 |
| Present compound 14-14 + pyraclostrobin | 5 + 50 | 100 |
| Present compound 14-14 + pyraclostrobin | 10 + 1 | 100 |
| Present compound 14-14 + picoxystrobin | 10 + 10 | 100 |
| Present compound 14-14 + picoxystrobin | 5 + 50 | 100 |
| Present compound 14-14 + picoxystrobin | 10 + 1 | 100 |
| Present compound 14-14 + prothioconazole | 10 + 10 | 100 |
| Present compound 14-14 + prothioconazole | 5 + 50 | 100 |
| Present compound 14-14 + prothioconazole | 30 + 10 | 100 |
| Present compound 14-14 + fenpicoxamid | 10 + 10 | 100 |
| Present compound 14-14 + fenpicoxamid | 5 + 50 | 100 |
| Present compound 14-14 + fenpicoxamid | 30 + 10 | 100 |
| Present compound 14-14 + florylpicoxamid | 10 + 10 | 100 |
| Present compound 14-14 + florylpicoxamid | 5 + 50 | 100 |
| Present compound 14-14 + florylpicoxamid | 30 + 10 | 100 |
| Present compound 14-14 + 3-(2-methylphenyl)butan-2-yl N-[(3-hydroxy-4- | 10 + 10 | 100 |
| methoxypyridin-2-yl)carbonyl]alaninate | | |
| Present compound 14-14 + 3-(2-methylphenyl)butan-2-yl N-[(3-hydroxy-4- | 5 + 50 | 100 |
| methoxypyridin-2-yl)carbonyl]alaninate | | |
| Present compound 14-14 + 3-(2-methylphenyl)butan-2-yl N-[(3-hydroxy-4- | 10 + 1 | 100 |
| methoxypyridin-2-yl)carbonyl]alaninate | | |
| Present compound 14-14 + 3-(2,4-dimethylphenyl)butan-2-yl N-[(3-acetoxy-4- | 10 + 10 | 100 |
| methoxypyridin-2-yl)carbonyl]alaninate | | |
| Present compound 14-14 + 3-(2,4-dimethylphenyl)butan-2-yl N-[(3-acetoxy-4- | 5 + 50 | 100 |
| methoxypyridin-2-yl)carbonyl]alaninate | | |
| Present compound 14-14 + 3-(2,4-dimethylphenyl)butan-2-yl N-[(3-acetoxy-4- | 10 + 1 | 100 |
| methoxypyridin-2-yl)carbonyl]alaninate | | |
| Present compound 14-14 + 3-(4-fluoro-2-methylphenyl)butan-2-yl N-({3-[(2- | 10 + 10 | 100 |
| methylpropanoyl)oxy]-4-methoxypyridin-2- | | |
| yl}carbonyl)alaninate | | |
| Present compound 14-14 + 3-(4-fluoro-2-methylphenyl)butan-2-yl N-({3-[(2- | 5 + 50 | 100 |
| methylpropanoyl)oxy]-4-methoxypyridin-2- | | |
| yl}carbonyl)alaninate | | |
| Present compound 14-14 + 3-(4-fluoro-2-methylphenyl)butan-2-yl N-({3-[(2- | 10 + 1 | 100 |
| methylpropanoyl)oxy]-4-methoxypyridin-2- | | |
| yl}carbonyl)alaninate | | |
| Present compound 14-14 + 3-(4-methoxy-2-methylphenyl)butan-2-yl N-[(3- | 10 + 10 | 100 |
| hydroxy-4-methoxypyridin-2-yl)carbonyl]alaninate | | |
| Present compound 14-14 + 3-(4-methoxy-2-methylphenyl)butan-2-yl N-[(3- | 5 + 50 | 100 |
| hydroxy-4-methoxypyridin-2-yl)carbonyl]alaninate | | |
| Present compound 14-14 + 3-(4-methoxy-2-methylphenyl)butan-2-yl N-[(3- | 10 + 1 | 100 |
| hydroxy-4-methoxypyridin-2-yl)carbonyl]alaninate | | |

TABLE B19

| | Conc. | Control |
|---|---|---|
| Present composition | (ppm) | Effect |
| Present compound 14-8 + benzovindiflupyr | 10 + 10 | 100 |
| Present compound 14-8 + benzovindiflupyr | 3 + 30 | 100 |
| Present compound 14-8 + benzovindiflupyr | 10 + 1 | 100 |
| Present compound 14-8 + 3-(difluoromethyl)-N-(2,3-dihydro-1,1,3- | 10 + 10 | 100 |
| trimethyl-1H-inden-4-yl)-1-methyl-1H-pyrazole-4- | | |
| carboxamide | | |
| Present compound 14-8 + 3-(difluoromethyl)-N-(2,3-dihydro-1,1,3- | 3 + 30 | 100 |
| trimethyl-1H-inden-4-yl)-1-methyl-1H-pyrazole-4- | | |
| carboxamide | | |
| Present compound 14-8 + 3-(difluoromethyl)-N-(2,3-dihydro-1,1,3- | 10 + 1 | 100 |
| trimethyl-1H-inden-4-yl)-1-methyl-1H-pyrazole-4- | | |
| carboxamide | | |
| Present compound 14-8 + fluindapyr | 10 + 10 | 100 |
| Present compound 14-8 + fluindapyr | 3 + 30 | 100 |
| Present compound 14-8 + fluindapyr | 10 + 1 | 100 |
| Present compound 14-8 + fluxapyroxad | 10 + 10 | 100 |
| Present compound 14-8 + fluxapyroxad | 3 + 30 | 100 |
| Present compound 14-8 + fluxapyroxad | 10 + 1 | 100 |
| Present compound 14-8 + Compound of formula (1) | 10 + 10 | 100 |
| Present compound 14-8 + Compound of formula (1) | 3 + 30 | 100 |
| Present compound 14-8 + Compound of formula (1) | 10 + 1 | 100 |
| Present compound 14-8 + Compound of formula (2) | 10 + 10 | 100 |
| Present compound 14-8 + Compound of formula (2) | 3 + 30 | 100 |
| Present compound 14-8 + Compound of formula (2) | 10 + 1 | 100 |
| Present compound 14-8 + Compound of formula (3) | 10 + 10 | 100 |
| Present compound 14-8 + Compound of formula (3) | 3 + 30 | 100 |
| Present compound 14-8 + Compound of formula (3) | 10 + 1 | 100 |
| Present compound 14-8 + pyraclostrobin | 10 + 10 | 100 |
| Present compound 14-8 + pyraclostrobin | 5 + 50 | 100 |
| Present compound 14-8 + pyraclostrobin | 10 + 1 | 100 |
| Present compound 14-8 + picoxystrobin | 10 + 10 | 100 |
| Present compound 14-8 + picoxystrobin | 5 + 50 | 100 |
| Present compound 14-8 + picoxystrobin | 10 + 1 | 100 |
| Present compound 14-8 + prothioconazole | 10 + 10 | 100 |
| Present compound 14-8 prothioconazole | 5 + 50 | 100 |
| Present compound 14-8 prothioconazole | 30 + 10 | 100 |
| Present compound 14-8 + fenpicoxamid | 10 + 10 | 100 |
| Present compound 14-8 + fenpicoxamid | 5 + 50 | 100 |
| Present compound 14-8 + fenpicoxamid | 30 + 10 | 100 |
| Present compound 14-8 + florylpicoxamid | 10 + 10 | 100 |
| Present compound 14-8 + florylpicoxamid | 5 + 50 | 100 |
| Present compound 14-8 + florylpicoxamid | 30 + 10 | 100 |
| Present compound 14-8 + 3-(2-methylphenyl)butan-2-yl N-[(3-hydroxy-4- | 10 + 10 | 100 |
| methoxypyridin-2-yl)carbonyl]alaninate | | |
| Present compound 14-8 + 3-(2-methylphenyl)butan-2-yl N-[(3-hydroxy-4- | 5 + 50 | 100 |
| methoxypyridin-2-yl)carbonyl]alaninate | | |
| Present compound 14-8 + 3-(2-methylphenyl)butan-2-yl N-[(3-hydroxy-4- | 10 + 1 | 100 |
| methoxypyridin-2-yl)carbonyl]alaninate | | |
| Present compound 14-8 + 3-(2,4-dimethylphenyl)butan-2-yl N-[(3-acetoxy-4- | 10 + 10 | 100 |
| methoxypyridin-2-yl)carbonyl]alaninate | | |
| Present compound 14-8 + 3-(2,4-dimethylphenyl)butan-2-yl N-[(3-acetoxy-4- | 5 + 50 | 100 |
| methoxypyridin-2-yl)carbonyl]alaninate | | |
| Present compound 14-8 + 3-(2,4-dimethylphenyl)butan-2-yl N-[(3-acetoxy-4- | 10 + 1 | 100 |
| methoxypyridin-2-yl)carbonyl]alaninate | | |
| Present compound 14-8 + 3-(4-fluoro-2-methylphenyl)butan-2-yl N-({3-[(2- | 10 + 10 | 100 |
| methylpropanoyl)oxy]-4-methoxypyridin-2- | | |
| yl}carbonyl)alaninate | | |
| Present compound 14-8 + 3-(4-fluoro-2-methylphenyl)butan-2-yl N-({3-[(2- | 5 + 50 | 100 |
| methylpropanoyl)oxy]-4-methoxypyridin-2- | | |
| yl}carbonyl)alaninate | | |
| Present compound 14-8 + 3-(4-fluoro-2-methylphenyl)butan-2-yl N-({3-[(2- | 10 + 1 | 100 |
| methylpropanoyl)oxy]-4-methoxypyridin-2- | | |
| yl}carbonyl)alaninate | | |
| Present compound 14-8 + 3-(4-methoxy-2-methylphenyl)butan-2-yl N-[(3- | 10 + 10 | 100 |
| hydroxy-4-methoxypyridin-2-yl)carbonyl]alaninate | | |
| Present compound 14-8 + 3-(4-methoxy-2-methylphenyl)butan-2-yl N-[(3- | 5 + 50 | 100 |
| hydroxy-4-methoxypyridin-2-yl)carbonyl]alaninate | | |
| Present compound 14-8 + 3-(4-methoxy-2-methylphenyl)butan-2-yl N-[(3- | 10 + 1 | 100 |
| hydroxy-4-methoxypyridin-2-yl)carbonyl]alaninate | | |

### Test Example 2: Control Test Against Wheat Septoria Leaf Blotch Fungus (Septoria tritici)

Each of any one of the compound selected from the present compound T and the present compound B were diluted with DMSO so as to contain a predetermined concentration thereof, and 1 μL of the dilution mixtures were dispensed into titer plate (96 well), and thereafter, thereto was then dispensed 150 μL of a potato dextrose broth (PDB broth) to which conidia of Septoria tritici were inoculated in advance. This plate was cultured at 18° C. for 4 days, thereby allowing Septoria tritici to undergo proliferation, and the absorbance at 550 nm of each well of the titer plate was then measured to determine a degree of growth of Septoria tritici (which was “treated area”).

Control effect was calculated by the following “Equation 5” from the degree of growth.

Control effect=100×(X−Y)/X “Equation 5”
X: Degree of growth of fungus in the non-treated area
Y: Degree of growth of fungus in the treated area

Here the non-treated area represents an area wherein the same procedures are taken except for using the present compound T and the present compound B.

The results are shown in Table C1 to Table C6.

TABLE C1

| | Conc. | Control |
|---|---|---|
| Present composition | (ppm) | Effect |
| Present compound 4-40 + pydiflumetofen | 0.04 + 0.04 | 100 |
| Present compound 4-40 + pydiflumetofen | 0.008 + 0.04 | 100 |
| Present compound 4-40 + pydiflumetofen | 0.04 + 0.008 | 100 |
| Present compound 4-40 + mefentrifluconazole | 0.04 + 0.04 | 100 |
| Present compound 4-40 + mefentrifluconazole | 0.008 + 0.04 | 100 |
| Present compound 4-40 + mefentrifluconazole | 0.04 + 0.008 | 100 |
| Present compound 4-40 + metyltetraprole | 0.04 + 0.04 | 100 |
| Present compound 4-40 + metyltetraprole | 0.008 + 0.04 | 100 |
| Present compound 4-40 + metyltetraprole | 0.04 + 0.008 | 100 |
| Present compound 4-17 + pydiflumetofen | 0.04 + 0.04 | 100 |
| Present compound 4-17 + pydiflumetofen | 0.008 + 0.04 | 100 |
| Present compound 4-17 + pydiflumetofen | 0.04 + 0.008 | 100 |
| Present compound 4-17 + mefentrifluconazole | 0.04 + 0.04 | 100 |
| Present compound 4-17 + mefentrifluconazole | 0.008 + 0.04 | 100 |
| Present compound 4-17 + mefentrifluconazole | 0.04 + 0.008 | 100 |
| Present compound 4-17 + metyltetraprole | 0.04 + 0.04 | 100 |
| Present compound 4-17 + metyltetraprole | 0.008 + 0.04 | 100 |
| Present compound 4-17 + metyltetraprole | 0.04 + 0.008 | 100 |
| Present compound 4-1 + pydiflumetofen | 0.04 + 0.04 | 100 |
| Present compound 4-1 + pydiflumetofen | 0.008 + 0.04 | 100 |
| Present compound 4-1 + pydiflumetofen | 0.04 + 0.008 | 100 |
| Present compound 4-1 + mefentrifluconazole | 0.04 + 0.04 | 100 |
| Present compound 4-1 + mefentrifluconazole | 0.008 + 0.04 | 100 |
| Present compound 4-1 + mefentrifluconazole | 0.04 + 0.008 | 100 |
| Present compound 4-1 + metyltetraprole | 0.04 + 0.04 | 100 |
| Present compound 4-1 + metyltetraprole | 0.008 + 0.04 | 100 |
| Present compound 4-1 + metyltetraprole | 0.04 + 0.008 | 100 |
| Present compound 4-36 + pydiflumetofen | 0.04 + 0.04 | 100 |
| Present compound 4-36 + pydiflumetofen | 0.008 + 0.04 | 100 |
| Present compound 4-36 + pydiflumetofen | 0.04 + 0.008 | 100 |
| Present compound 4-36 + mefentrifluconazole | 0.04 + 0.04 | 100 |
| Present compound 4-36 + mefentrifluconazole | 0.008 + 0.04 | 100 |
| Present compound 4-36 + mefentrifluconazole | 0.04 + 0.008 | 100 |
| Present compound 4-36 + metyltetraprole | 0.04 + 0.04 | 100 |
| Present compound 4-36 + metyltetraprole | 0.008 + 0.04 | 100 |
| Present compound 4-36 + metyltetraprole | 0.04 + 0.008 | 100 |

TABLE C2

| | Conc. | Control |
|---|---|---|
| Present composition | (ppm) | Effect |
| Present compound 4-6 + pydiflumetofen | 0.04 + 0.04 | 100 |
| Present compound 4-6 + pydiflumetofen | 0.008 + 0.04 | 100 |
| Present compound 4-6 + pydiflumetofen | 0.04 + 0.008 | 100 |
| Present compound 4-6 + mefentrifluconazole | 0.04 + 0.04 | 100 |
| Present compound 4-6 + mefentrifluconazole | 0.008 + 0.04 | 100 |
| Present compound 4-6 + mefentrifluconazole | 0.04 + 0.008 | 100 |
| Present compound 4-6 + metyltetraprole | 0.04 + 0.04 | 100 |
| Present compound 4-6 + metyltetraprole | 0.008 + 0.04 | 100 |
| Present compound 4-6 + metyltetraprole | 0.04 + 0.008 | 100 |
| Present compound 4-38 + pydiflumetofen | 0.04 + 0.04 | 100 |
| Present compound 4-38 + pydiflumetofen | 0.008 + 0.04 | 100 |
| Present compound 4-38 + pydiflumetofen | 0.04 + 0.008 | 100 |
| Present compound 4-38 + mefentrifluconazole | 0.04 + 0.04 | 100 |
| Present compound 4-38 + mefentrifluconazole | 0.008 + 0.04 | 100 |
| Present compound 4-38 + mefentrifluconazole | 0.04 + 0.008 | 100 |
| Present compound 4-38 + metyltetraprole | 0.04 + 0.04 | 100 |
| Present compound 4-38 + metyltetraprole | 0.008 + 0.04 | 100 |
| Present compound 4-38 + metyltetraprole | 0.04 + 0.008 | 100 |
| Present compound 4-12 + pydiflumetofen | 0.04 + 0.04 | 100 |
| Present compound 4-12 + pydiflumetofen | 0.008 + 0.04 | 100 |
| Present compound 4-12 + pydiflumetofen | 0.04 + 0.008 | 100 |
| Present compound 4-12 + mefentrifluconazole | 0.04 + 0.04 | 100 |
| Present compound 4-12 + mefentrifluconazole | 0.008 + 0.04 | 100 |
| Present compound 4-12 + mefentrifluconazole | 0.04 + 0.008 | 100 |
| Present compound 4-12 + metyltetraprole | 0.04 + 0.04 | 100 |
| Present compound 4-12 + metyltetraprole | 0.008 + 0.04 | 100 |
| Present compound 4-12 + metyltetraprole | 0.04 + 0.008 | 100 |

TABLE C3

| | Conc. | Control |
|---|---|---|
| Present composition | (ppm) | Effect |
| Present compound 4-14 + pydiflumetofen | 0.04 + 0.04 | 100 |
| Present compound 4-14 + pydiflumetofen | 0.008 + 0.04 | 100 |
| Present compound 4-14 + pydiflumetofen | 0.04 + 0.008 | 100 |
| Present compound 4-14 + mefentrifluconazole | 0.04 + 0.04 | 100 |
| Present compound 4-14 + mefentrifluconazole | 0.008 + 0.04 | 100 |
| Present compound 4-14 + mefentrifluconazole | 0.04 + 0.008 | 100 |
| Present compound 4-14 + metyltetraprole | 0.04 + 0.04 | 100 |
| Present compound 4-14 + metyltetraprole | 0.008 + 0.04 | 100 |
| Present compound 4-14 + metyltetraprole | 0.04 + 0.008 | 100 |
| Present compound 4-13 + pydiflumetofen | 0.04 + 0.04 | 100 |
| Present compound 4-13 + pydiflumetofen | 0.008 + 0.04 | 100 |
| Present compound 4-13 + pydiflumetofen | 0.04 + 0.008 | 100 |
| Present compound 4-13 + mefentrifluconazole | 0.04 + 0.04 | 100 |
| Present compound 4-13 + mefentrifluconazole | 0.008 + 0.04 | 100 |
| Present compound 4-13 + mefentrifluconazole | 0.04 + 0.008 | 100 |
| Present compound 4-13 + metyltetraprole | 0.04 + 0.04 | 100 |
| Present compound 4-13 + metyltetraprole | 0.008 + 0.04 | 100 |
| Present compound 4-13 + metyltetraprole | 0.04 + 0.008 | 100 |
| Present compound 4-15 + pydiflumetofen | 0.04 + 0.04 | 100 |
| Present compound 4-15 + pydiflumetofen | 0.008 + 0.04 | 100 |
| Present compound 4-15 + pydiflumetofen | 0.04 + 0.008 | 100 |
| Present compound 4-15 + mefentrifluconazole | 0.04 + 0.04 | 100 |
| Present compound 4-15 + mefentrifluconazole | 0.008 + 0.04 | 100 |
| Present compound 4-15 + mefentrifluconazole | 0.04 + 0.008 | 100 |
| Present compound 4-15 + metyltetraprole | 0.04 + 0.04 | 100 |
| Present compound 4-15 + metyltetraprole | 0.008 + 0.04 | 100 |
| Present compound 4-15 + metyltetraprole | 0.04 + 0.008 | 100 |

TABLE C4

| | Conc. | Control |
|---|---|---|
| Present composition | (ppm) | Effect |
| Present compound 14-4 + pydiflumetofen | 0.04 + 0.04 | 100 |
| Present compound 14-4 + pydiflumetofen | 0.008 + 0.04 | 100 |
| Present compound 14-4 + pydiflumetofen | 0.04 + 0.008 | 100 |
| Present compound 14-4 + mefentrifluconazole | 0.04 + 0.04 | 100 |
| Present compound 14-4 + mefentrifluconazole | 0.008 + 0.04 | 100 |
| Present compound 14-4 + mefentrifluconazole | 0.04 + 0.008 | 100 |
| Present compound 14-4 + metyltetraprole | 0.04 + 0.04 | 100 |
| Present compound 14-4 + metyltetraprole | 0.008 + 0.04 | 100 |
| Present compound 14-4 + metyltetraprole | 0.04 + 0.008 | 100 |
| Present compound 14-7 + pydiflumetofen | 0.04 + 0.04 | 100 |
| Present compound 14-7 + pydiflumetofen | 0.008 + 0.04 | 100 |
| Present compound 14-7 + pydiflumetofen | 0.04 + 0.008 | 100 |
| Present compound 14-7 + mefentrifluconazole | 0.04 + 0.04 | 100 |
| Present compound 14-7 + mefentrifluconazole | 0.008 + 0.04 | 100 |
| Present compound 14-7 + mefentrifluconazole | 0.04 + 0.008 | 100 |
| Present compound 14-7 + metyltetraprole | 0.04 + 0.04 | 100 |
| Present compound 14-7 + metyltetraprole | 0.008 + 0.04 | 100 |
| Present compound 14-7 + metyltetraprole | 0.04 + 0.008 | 100 |
| Present compound 14-6 + pydiflumetofen | 0.04 + 0.04 | 100 |
| Present compound 14-6 + pydiflumetofen | 0.008 + 0.04 | 100 |
| Present compound 14-6 + pydiflumetofen | 0.04 + 0.008 | 100 |
| Present compound 14-6 + mefentrifluconazole | 0.04 + 0.04 | 100 |
| Present compound 14-6 + mefentrifluconazole | 0.008 + 0.04 | 100 |
| Present compound 14-6 + mefentrifluconazole | 0.04 + 0.008 | 100 |
| Present compound 14-6 + metyltetraprole | 0.04 + 0.04 | 100 |
| Present compound 14-6 + metyltetraprole | 0.008 + 0.04 | 100 |
| Present compound 14-6 + metyltetraprole | 0.04 + 0.008 | 100 |
| Present compound 14-2 + pydiflumetofen | 0.04 + 0.04 | 100 |
| Present compound 14-2 + pydiflumetofen | 0.008 + 0.04 | 100 |
| Present compound 14-2 + pydiflumetofen | 0.04 + 0.008 | 100 |
| Present compound 14-2 + mefentrifluconazole | 0.04 + 0.04 | 100 |
| Present compound 14-2 + mefentrifluconazole | 0.008 + 0.04 | 100 |
| Present compound 14-2 + mefentrifluconazole | 0.04 + 0.008 | 100 |
| Present compound 14-2 + metyltetraprole | 0.04 + 0.04 | 100 |
| Present compound 14-2 + metyltetraprole | 0.008 + 0.04 | 100 |
| Present compound 14-2 + metyltetraprole | 0.04 + 0.008 | 100 |

TABLE C5

| | Conc. | Control |
|---|---|---|
| Present composition | (ppm) | Effect |
| Present compound 14-74 + pydiflumetofen | 0.04 + 0.04 | 100 |
| Present compound 14-74 + pydiflumetofen | 0.008 + 0.04 | 100 |
| Present compound 14-74 + pydiflumetofen | 0.04 + 0.008 | 100 |
| Present compound 14-74 + mefentrifluconazole | 0.04 + 0.04 | 100 |
| Present compound 14-74 + mefentrifluconazole | 0.008 + 0.04 | 100 |
| Present compound 14-74 + mefentrifluconazole | 0.04 + 0.008 | 100 |
| Present compound 14-74 + metyltetraprole | 0.04 + 0.04 | 100 |
| Present compound 14-74 + metyltetraprole | 0.008 + 0.04 | 100 |
| Present compound 14-74 + metyltetraprole | 0.04 + 0.008 | 100 |
| Present compound 14-10 + pydiflumetofen | 0.04 + 0.04 | 100 |
| Present compound 14-10 + pydiflumetofen | 0.008 + 0.04 | 100 |
| Present compound 14-10 + pydiflumetofen | 0.04 + 0.008 | 100 |
| Present compound 14-10 + mefentrifluconazole | 0.04 + 0.04 | 100 |
| Present compound 14-10 + mefentrifluconazole | 0.008 + 0.04 | 100 |
| Present compound 14-10 + mefentrifluconazole | 0.04 + 0.008 | 100 |
| Present compound 14-10 + metyltetraprole | 0.04 + 0.04 | 100 |
| Present compound 14-10 + metyltetraprole | 0.008 + 0.04 | 100 |
| Present compound 14-10 + metyltetraprole | 0.04 + 0.008 | 100 |
| Present compound 14-9 + pydiflumetofen | 0.04 + 0.04 | 100 |
| Present compound 14-9 + pydiflumetofen | 0.008 + 0.04 | 100 |
| Present compound 14-9 + pydiflumetofen | 0.04 + 0.008 | 100 |
| Present compound 14-9 + mefentrifluconazole | 0.04 + 0.04 | 100 |
| Present compound 14-9 + mefentrifluconazole | 0.008 + 0.04 | 100 |
| Present compound 14-9 + mefentrifluconazole | 0.04 + 0.008 | 100 |
| Present compound 14-9 + metyltetraprole | 0.04 + 0.04 | 100 |
| Present compound 14-9 + metyltetraprole | 0.008 + 0.04 | 100 |
| Present compound 14-9 + metyltetraprole | 0.04 + 0.008 | 100 |
| Present compound 14-14 + pydiflumetofen | 0.04 + 0.04 | 100 |
| Present compound 14-14 + pydiflumetofen | 0.008 + 0.04 | 100 |
| Present compound 14-14 + pydiflumetofen | 0.04 + 0.008 | 100 |
| Present compound 14-14 + mefentrifluconazole | 0.04 + 0.04 | 100 |
| Present compound 14-14 + mefentrifluconazole | 0.008 + 0.04 | 100 |
| Present compound 14-14 + mefentrifluconazole | 0.04 + 0.008 | 100 |
| Present compound 14-14 + metyltetraprole | 0.04 + 0.04 | 100 |
| Present compound 14-14 + metyltetraprole | 0.008 + 0.04 | 100 |
| Present compound 14-14 + metyltetraprole | 0.04 + 0.008 | 100 |

TABLE C6

| | Conc. | Control |
|---|---|---|
| Present composition | (ppm) | Effect |
| Present compound 14-8 + pydiflumetofen | 0.04 + 0.04 | 100 |
| Present compound 14-8 + pydiflumetofen | 0.008 + 0.04 | 100 |
| Present compound 14-8 + pydiflumetofen | 0.04 + 0.008 | 100 |
| Present compound 14-8 + mefentrifluconazole | 0.04 + 0.04 | 100 |
| Present compound 14-8 + mefentrifluconazole | 0.008 + 0.04 | 100 |
| Present compound 14-8 + mefentrifluconazole | 0.04 + 0.008 | 100 |
| Present compound 14-8 + metyltetraprole | 0.04 + 0.04 | 100 |
| Present compound 14-8 + metyltetraprole | 0.008 + 0.04 | 100 |
| Present compound 14-8 + metyltetraprole | 0.04 + 0.008 | 100 |

### INDUSTRIAL APPLICABILITY

The present compound or the composition A shows a control effect against a soybean rust fungus having an amino acid substitution of F129L on mitochondrial cytochrome b protein.

Also the composition for controlling plant diseases of the present invention can control plant diseases.

1. A method for controlling a soybean rust fungus having an amino acid substitution of F129L on mitochondrial cytochrome b protein, which comprises applying an effective amount of a compound represented by formula (I): 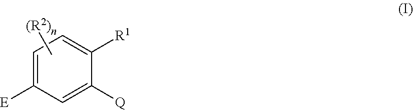
[wherein,
a combination of Q and E represents
a combination wherein
Q represents a group represented by Q1, or a group represented by Q2,
E represents a C1-C6 chain hydrocarbon group which may be optionally substituted with one or more substituents selected from Group A, a C3-C6 cycloalkyl group which may be optionally substituted with one or more substituents selected from Group B, R⁴R⁵C═C(R⁶)—, R¹⁵—CR¹⁶R³⁶—O—N═C(R⁷)—, R⁸R⁹C═NO—CH₂—, R¹²O—N═C(R⁷)—C(R⁹)═N—O—CH₂—, R¹²C(O)—C(R⁹)═NO—CH₂—, R¹²C(═N—O—R⁷)—C(R⁹)═N—O—CH₂—, R³⁴R³⁵C═N—N═C(R¹⁰)—R¹²R⁹N—N═C(R⁷)—, R¹²SC(R⁷)═N—, R¹²SC(SR⁷)═N—, R¹²N═C(R⁷)—S—CH₂—, R¹²N═C(SR⁷)—S—CH₂—, R¹²O—N═C(R⁷)—S—CH₂—, R¹²O—N═C(SR⁷)—S—CH₂—, R¹²R⁹N—C(S)—O—CH₂—, or R¹⁴O—; or
a combination wherein
Q represents a group represented by Q3, a group represented by Q4, a group represented by Q5, a group represented by Q6, a group represented by Q7, or a group represented by Q8,
E represents a C1-C6 chain hydrocarbon group which may be optionally substituted with one or more substituents selected from Group A, a C3-C6 cycloalkyl group, a C5-C6 cycloalkenyl group {the C3-C6 cycloalkyl group, and the C5-C6 cycloalkenyl group may be optionally substituted with one or more substituents selected from Group B}, a C6-C10 aryl group, a five- to ten-membered aromatic heterocyclic group {the C6-C10 aryl group and the five- to ten-membered aromatic heterocyclic group may be optionally substituted with one or more substituents selected from Group D}, R⁴R⁵C═C(R⁶)—, R¹³O—N═C(R⁷)—, R⁸R⁹C═N—O—CH₂—, R¹²O—N═C(R⁷)—C(R⁹)═N—O—CH₂—, R¹²C(O)—C(R⁹)═NO—CH₂—, R¹²C(═N—O—R⁷)—C(R⁹)═NO—CH₂—, R³⁴R³⁵C═N—N═C(R¹⁰)—, R¹²R⁹N—N═C(R⁷)—, R¹²SC(R⁷)═N—, R¹²SC(SR⁷)═N—, R¹²N═C(R⁷)—S—CH₂—, R¹²N═C(SR⁷)—S—CH₂—, R¹²O—N═C(R⁷)—S—CH₂—, R¹²O—N═C(SR⁷)—S—CH₂—, R¹²R⁹N—C(S)—O—CH₂—, R¹⁹O—, R²⁰—C≡C—, or R¹⁸—N═C(R⁷)—,
each of the group represented by Q1, the group represented by Q2, the group represented by Q3, the group represented by Q4, the group represented by Q5, the group represented by Q6, the group represented by Q7, and the group represented by Q8 represents the following group respectively (where • represents a binding site to a benzene ring), 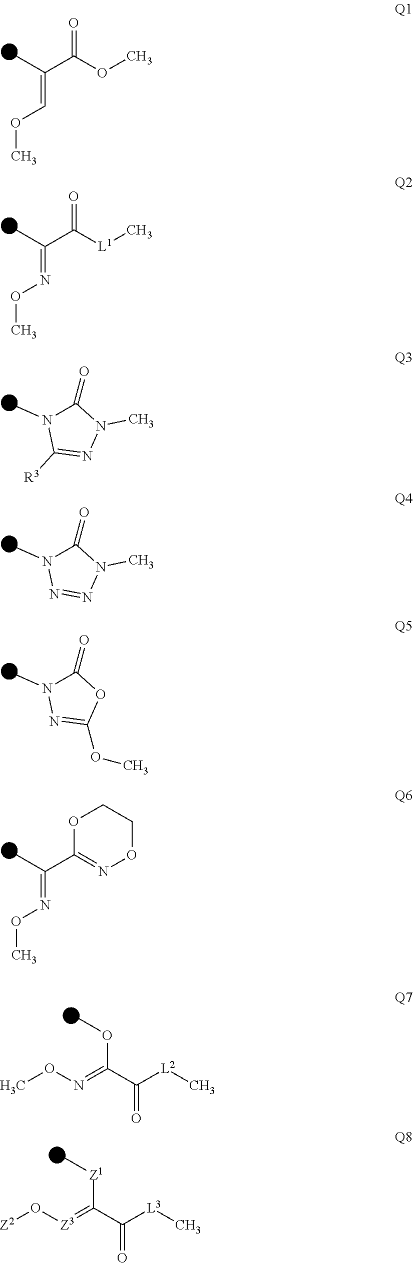
L¹, L² and L³ are identical to or different from each other and represent an oxygen atom or NH,
Z¹ represents a single bond, CH₂, an oxygen atom or NCH₃,
Z² represents a methyl group which is substituted with one or more fluorine atoms,
Z³ represents CH or a nitrogen atom,
R¹ represents a C1-C3 chain hydrocarbon group, a C1-C3 alkoxy group {the C1-C3 chain hydrocarbon group, and the C1-C3 alkoxy group may be optionally substituted with one or more halogen atoms}, a cyclopropyl group, a halogen atom, or a hydrogen atom,
n is 0, 1, 2 or 3,
when n is 2 or 3, a plural of R² may be identical to or different from each other,
R² represents a C1-C3 chain hydrocarbon group which may be optionally substituted with one or more halogen atoms, or a C1-C3 alkoxy group {the C1-C3 chain hydrocarbon group, and the C1-C3 alkoxy group may be optionally substituted with one or more halogen atoms}, a cyclopropyl group, or a halogen atom,
R³ represents a C1-C3 alkoxy group which may be optionally substituted with one or more halogen atoms, or a C1-C3 chain hydrocarbon group which may be optionally substituted with one or more halogen atoms,
R⁴ and R⁶ are identical to or different from each other and represent a C1-C3 chain hydrocarbon group which may be optionally substituted with one or more halogen atoms, a cyclopropyl group, a halogen atom, a cyano group, or a hydrogen atom,
R⁵ represents a C1-C6 chain hydrocarbon group which may be optionally substituted with one or more substituents selected from Group A, a C3-C6 cycloalkyl group which may be optionally substituted with one or more substituents selected from Group B, a C6-C10 aryl group, a five- to ten-membered aromatic heterocyclic group {the C6-C10 aryl group and the five- to ten-membered aromatic heterocyclic group may be optionally substituted with one or more substituents selected from Group D}, a halogen atom, a cyano group, or a hydrogen atom,
R⁸, R¹², R¹³, R¹⁵, R¹⁸, R²⁰, and R³⁴ are identical to or different from each other and represent a C1-C6 chain hydrocarbon group which may be optionally substituted with one or more substituents selected from Group A, a C3-C10 alicyclic hydrocarbon group which may be optionally substituted with one or more substituents selected from Group E, a C6-C10 aryl group, or a five- to ten-membered aromatic heterocyclic group {the C6-C10 aryl group and the five- to ten-membered aromatic heterocyclic group may be optionally substituted with one or more substituents selected from Group D},
R⁷, R⁹, R¹⁰ and R³⁵ are identical to or different from each other and represent a C1-C3 chain hydrocarbon group which may be optionally substituted with one or more halogen atoms, a cyclopropyl group, or a hydrogen atom,
R⁴ and R⁵ may combine together with a carbon atom to which they are attached to form a C3-C10 alicyclic hydrocarbon group or a three- to ten-membered non-aromatic heterocyclic group {the C3-C10 alicyclic hydrocarbon group and the three- to ten-membered non-aromatic heterocyclic group may be optionally substituted with one or more substituents selected from Group E},
R⁸ and R⁹ may combine together with a carbon atom to which they are attached to form a C3-C10 alicyclic hydrocarbon group or a three- to ten-membered non-aromatic heterocyclic group {the C3-C10 alicyclic hydrocarbon group and the three- to ten-membered non-aromatic heterocyclic group may be optionally substituted with one or more substituents selected from Group E},
R³⁴ and R³⁵ may combine together with a carbon atom to which they are attached to form a C3-C10 alicyclic hydrocarbon group or a three- to ten-membered non-aromatic heterocyclic group {the C3-C10 alicyclic hydrocarbon group and the three- to ten-membered non-aromatic heterocyclic group may be optionally substituted with one or more substituents selected from Group E},
R¹⁴ represents a C6-C10 aryl group, a five- to ten-membered aromatic heterocyclic group {the C6-C10 aryl group and the five- to ten-membered aromatic heterocyclic group may be optionally substituted with one or more substituents selected from Group D}, R¹⁷R⁷NC(S)—, R¹⁷O—C(O)—, or R¹⁷C(O)—,
R¹⁵ represents a C6-C10 aryl group, or a five- to ten-membered aromatic heterocyclic group {the C6-C10 aryl group and the five- to ten-membered aromatic heterocyclic group may be optionally substituted with one or more substituents selected from Group C},
R¹⁶ represents a C1-C3 chain hydrocarbon group which may be optionally substituted with one or more halogen atoms, or a cyclopropyl group,
R¹⁷ represents a C1-C6 chain hydrocarbon group which may be optionally substituted with one or more substituents selected from Group A, a C3-C6 cycloalkyl group which may be optionally substituted with one or more substituents selected from Group B, a C6-C10 aryl group, or a five- to ten-membered aromatic heterocyclic group {the C6-C10 aryl group and the five- to ten-membered aromatic heterocyclic group may be optionally substituted with one or more substituents selected from Group C},
R¹⁹ represents a C6-C10 aryl group, a five- to ten-membered aromatic heterocyclic group {the C6-C10 aryl group and the five- to ten-membered aromatic heterocyclic group may be optionally substituted with one or more substituents selected from Group D}, a methyl group which is substituted with one or more substituents selected from Group F, a C2-C6 chain hydrocarbon group which may be optionally substituted with one or more substituents selected from Group F, a C3-C4 cycloalkyl group which may be optionally substituted with one or more substituents selected from Group B, R¹⁷R⁷NC(S)—, R¹⁷O—C(O)—, R¹⁷C(O)—, or R¹⁷R⁷NC(O)—,
R³⁶ represents a C1-C3 chain hydrocarbon group which may be optionally substituted with one or more halogen atoms, a cyclopropyl group, or a hydrogen atom.
Group A is a group consisting of a C3-C6 cycloalkyl group {the C3-C6 cycloalkyl group may be optionally substituted with one or more substituents selected from Group B}, a C1-C4 alkoxy group, a C1-C4 alkylthio group {the C1-C4 alkoxy group and the C1-C4 alkylthio group may be optionally substituted with one or more substituents selected from Group F}, a halogen atom, a cyano group, a nitro group, a hydroxy group, an oxo group, a thioxo group, a phenoxy group, a C6-C10 aryl group, and a five- to ten-membered aromatic heterocyclic group {the phenoxy group, the C6-C10 aryl group and the five- to ten-membered aromatic heterocyclic group may be optionally substituted with one or more substituents selected from Group D}.
Group B is a group consisting of a C1-C3 chain hydrocarbon group which may be optionally substituted with one or more halogen atoms, a halogen atom, and a cyano group.
Group C is a group consisting of a C1-C6 chain hydrocarbon group, a C1-C6 alkoxy group, a C1-C6 alkylthio group {the C1-C6 chain hydrocarbon group, the C1-C6 alkoxy group and the C1-C6 alkylthio group may be optionally substituted with one or more substituents selected from Group F}, a C3-C6 cycloalkyl group {the C3-C6 cycloalkyl group may be optionally substituted with one or more substituents selected from Group B}, a halogen atom, a cyano group, a nitro group, and a hydroxy group.
Group D is a group consisting of a C1-C6 chain hydrocarbon group which may be optionally substituted with one or more substituents selected from Group F, a C3-C6 cycloalkyl group which may be optionally substituted with one or more substituents selected from Group B, a C1-C6 alkylthio group which may be optionally substituted with one or more substituents selected from Group F, OR¹¹, C(O)R¹¹, C(O)OR¹¹, NR¹¹R⁷, C(R⁷)═N—OR¹¹, a phenyl group, a naphthyl group, a five- to six-membered aromatic heterocyclic group {the phenyl group, the naphthyl group, and the five- to six-membered aromatic heterocyclic group may be optionally substituted with one or more substituents selected from Group C}, a halogen atom, a cyano group, and a nitro group.
R¹¹ represents a C1-C6 chain hydrocarbon group which may be optionally substituted with one or more substituents selected from Group A, a C3-C6 cycloalkyl group which may be optionally substituted with one or more substituents selected from Group B, a phenyl group, a naphthyl group, a five- to six-membered aromatic heterocyclic group {the phenyl group, the naphthyl group, and the five- to six-membered aromatic heterocyclic group may be optionally substituted with one or more substituents selected from Group C}, or a hydrogen atom.
Group E is a group consisting of an oxo group, a thioxo group, a C1-C3 chain hydrocarbon group, a C1-C3 alkoxy group {the C1-C3 chain hydrocarbon group and the C1-C3 alkoxy group may be optionally substituted with one or more halogen atoms}, a halogen atom, and a cyano group.
Group F is a group consisting of a C3-C4 cycloalkyl group, a halogen atom, and a C1-C3 alkoxy group],
or its N oxide or an agriculturally acceptable salt thereof.

2. The method according to claim 1 wherein the compound represented by formula (I), or its N oxide or an agriculturally acceptable salt thereof represents a compound wherein
Q represents a group represented by Q1, a group represented by Q2, a group represented by Q3, a group represented by Q4, a group represented by Q5, a group represented by Q6, a group represented by Q7, or a group represented by Q8,
E represents R⁴R⁵C═C(R⁶)—, R¹⁵—CR¹⁶R³⁶—O—N═C(R⁷)—, R⁸R⁹C═NO—CH₂—, R³⁴R³⁵C═N—N═C(R¹⁰)—, or R¹⁴O—,
R⁴ and R⁶ are identical to or different from each other and represent a C1-C3 alkyl group, a halogen atom, or a hydrogen atom,
R⁵ represents a C1-C6 chain hydrocarbon group which may be optionally substituted with one or more substituents selected from Group J, a C3-C6 cycloalkyl group which may be optionally substituted with one or more halogen atoms, or a phenyl group which may be optionally substituted with one or more substituents selected from Group G,
R⁷, R⁹ and R³⁵ are identical to or different from each other and represent a C1-C3 alkyl group, or a hydrogen atom,
R⁸ and R³⁴ are identical to or different from each other and represent a phenyl group, an indanyl group, a tetrahydronaphthyl group, or a five- to six-aromatic heterocyclic group {the phenyl group, the indanyl group, the tetrahydronaphthyl group, and the five- to six-aromatic heterocyclic group may be optionally substituted with one or more substituents selected from Group G},
R⁴ and R⁵ may combine together with a carbon atom to which they are attached to form a C3-C10 alicyclic hydrocarbon group, or a three- to ten-membered non-aromatic heterocyclic group {the C3-C10 alicyclic hydrocarbon group and the three- to ten-membered non-aromatic heterocyclic group may be optionally substituted with one or more substituents selected from Group E},
R⁸ and R⁹ may combine together with a carbon atom to which they are attached to form a C3-C10 alicyclic hydrocarbon group or a three- to ten-membered non-aromatic heterocyclic group {the C3-C10 alicyclic hydrocarbon group and the three- to ten-membered non-aromatic heterocyclic group may be optionally substituted with one or more substituents selected from Group E},
R³⁴ and R³⁵ may combine together with a carbon atom to which they are attached to form a C3-C10 alicyclic hydrocarbon group or a three- to ten-membered non-aromatic heterocyclic group {the C3-C10 alicyclic hydrocarbon group and the three- to ten-membered non-aromatic heterocyclic group may be optionally substituted with one or more substituents selected from Group E},
R¹⁴ and R¹⁵ are identical to or different from each other and represent a phenyl group, or a five- to six-membered aromatic heterocyclic group {the a phenyl group and the five- to six-membered aromatic heterocyclic group may be optionally substituted with one or more substituents selected from Group G},
R¹⁶ represents a C1-C3 alkyl group, and
R³⁶ represents a hydrogen atom,
Group G is a group consisting of a C1-C3 chain hydrocarbon group, a C1-C3 alkoxy group {the C1-C3 chain hydrocarbon group and the C1-C3 alkoxy group may be optionally substituted with one or more halogen atoms}, a halogen atom, and a cyano group.
Group J is a group consisting of a halogen atom and a cyclopropyl group,
or its N oxide or agriculturally acceptable salt thereof.

3. The method according to claim 2 wherein the compound represented by formula (I), or its N oxide or an agriculturally acceptable salt thereof represents a compound wherein
E represents R¹⁵—CR¹⁶R³⁶—O—N═C(R⁷)—, R⁸R⁹C═N—O—CH₂—, or R¹⁴O—,
R⁷ and R⁸ are identical to or different from each other and represent a C1-C3 alkyl group or a hydrogen atom,
R⁸, R¹⁴ and R¹⁵ are identical to or different from each other and represent a phenyl group which may be optionally substituted with one or more substituents selected from Group G,
R¹⁶ represents a C1-C3 alkyl group, and
R³⁶ represents a hydrogen atom,
or its N oxide or agriculturally acceptable salt thereof.

4. The method according to claim 1 wherein the compound respected by formula (I) is a compound wherein
E represents R¹⁴O—,
R¹⁴ represents a thiazolyl group which may be optionally substituted with one or more substituents selected from Group H, or a thienyl group which may be optionally substituted with one or more halogen atoms,
Group H is a group consisting of a C1-C3 chain hydrocarbon group which may be optionally substituted with one or more substituents selected from Group F, a C3-C6 cycloalkyl group, a phenyl group {the C3-C6 cycloalkyl group and the phenyl group may be optionally substituted with one or more halogen atoms}, and a halogen atom.

5. The method according to claim 1 wherein the compound represented by formula (I), or its N oxide or an agriculturally acceptable salt thereof represents a compound wherein
Q represents a group represented by Q1 or a group represented by Q2,
R¹ represents a methyl group, or a halogen atom, and
n is 0,
or its N oxide or agriculturally acceptable salt thereof.

6. The method according to claim 1 wherein the compound represented by formula (I), or its N oxide or an agriculturally acceptable salt thereof represents a compound wherein
Q represents a group represented by Q1,
R¹ represents a methyl group, and
n is 0,
or its N oxide or agriculturally acceptable salt thereof.

7. (canceled)

8. A compound represented by formula (II): 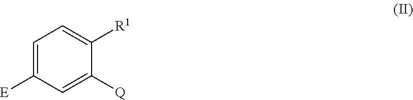
[wherein
R¹ represents a methyl group,
Q represents a group represented by Q1 or a group represented by Q2 (where • represents a binding site to a benzene ring), 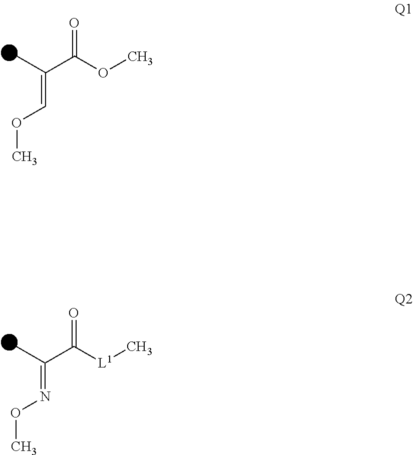
L¹ represents an oxygen atom or NH,
E represents R³⁴R³⁵C═N—N═C(R¹⁰)—, R⁸R⁹C═N—O—CH₂—, R¹⁵—CH(R¹⁶)—O—N═C(R⁷)—, or R¹⁹O—,
R³⁴ represents a C1-C6 chain hydrocarbon group which may be optionally substituted with one or more substituents selected from Group J, a C3-C10 alicyclic hydrocarbon group which may be optionally substituted with one or more substituents selected from Group E, a phenyl group, or a five- to six-membered aromatic heterocyclic group {the phenyl group and the five- to six-membered aromatic heterocyclic group may be optionally substituted with one or more substituents selected from Group G},
R⁹ and R³⁵ are identical to or different from each other and represent a C1-C3 alkyl group, or a hydrogen atom,
R⁷, R¹⁰ and R¹⁶ are identical to or different from each other and represent a C1-C3 alkyl group,
R⁸ represents an indanyl group, a tetrahydronaphthyl group, a dihydrobenzofuranyl group, or a dihydrobenzopyranyl group {the indanyl group, the tetrahydronaphthyl group, the dihydrobenzofuranyl group, and the dihydrobenzopyranyl group may be optionally substituted with one or more substituents selected from Group G},
R⁸ and R⁹ may combine together with a carbon atom to which they are attached to form a C3-C10 alicyclic hydrocarbon group or a three- to ten-membered non-aromatic heterocyclic group {the C3-C10 alicyclic hydrocarbon group and the three- to ten-membered non-aromatic heterocyclic group may be optionally substituted with one or more substituents selected from Group E},
R³⁴ and R³⁵ may combine together with a carbon atom to which they are attached to form a C3-C10 alicyclic hydrocarbon group or a three- to ten-membered non-aromatic heterocyclic group {the C3-C10 alicyclic hydrocarbon group and the three- to ten-membered non-aromatic heterocyclic group may be optionally substituted with one or more substituents selected from Group E},
R¹⁵ represents a phenyl group, or a five- to six-membered aromatic heterocyclic group {the phenyl group and the five- to six-membered aromatic heterocyclic group may be optionally substituted with one or more substituents selected from Group G},
R¹⁹ represents a phenyl group which is substituted at its 2-position with substituent(s) selected from Group L, a phenyl group which is substituted at its 3-position with substituent(s) selected from Group M, a phenyl group which is substituted at its 4-position with substituent(s) selected from Group N, a phenyl group which is substituted with two or more substituents selected from Group P (where the two or more substituents selected from Group P may be identical to or different from each other), a pyridyl group which may be optionally substituted with substituent(s) selected from Group P, a pyrazolyl group which may be optionally substituted with substituent(s) selected from Group G, or a pyrimidinyl group which may be optionally substituted with substituent(s) selected from Group Q.
Group E is a group consisting of an oxo group, a thioxo group, a C1-C3 chain hydrocarbon group, a C1-C3 alkoxy group {the C1-C3 chain hydrocarbon group and the C1-C3 alkoxy group may be optionally substituted with one or more halogen atoms}, a halogen atom, and a cyano group.
Group G is a group consisting of a C1-C3 chain hydrocarbon group, a C1-C3 alkoxy group {the C1-C3 chain hydrocarbon group and the C1-C3 alkoxy group may be optionally substituted with one or more halogen atoms}, a halogen atom, and a cyano group.
Group J is a group consisting of a halogen atom and a cyclopropyl group.
Group L is a group consisting of a C2-C4 chain hydrocarbon group, a C1-C4 alkoxy group {the C2-C4 chain hydrocarbon group and the C1-C4 alkoxy group may be optionally substituted with one or more halogen atoms}, a cyclopropyl group, a halogen atom, and a cyano group.
Group M is a group consisting of a C2-C3 chain hydrocarbon group, a C2-C3 alkoxy group {the C2-C3 chain hydrocarbon group and the C2-C3 alkoxy group may be optionally substituted with one or more halogen atoms}, a trifluoromethyl group, a cyclopropyl group, and a chlorine atom.
Group N is a group consisting of a C1-C3 alkyl group which is substituted with one or more fluorine atoms, a methoxy group, a cyclopropyl group, a halogen atom, and a cyano group.
Group P is a group consisting of a C2-C3 chain hydrocarbon group, a C1-C3 alkoxy group {the C2-C3 chain hydrocarbon group and the C1-C3 alkoxy group may be optionally substituted with one or more halogen atoms}, a halogen atom, and a cyano group.
Group Q is a group consisting of a C1-C3 chain hydrocarbon group which may be optionally substituted with one or more halogen atoms, a C1-C3 alkoxy group, a halogen atom, and a cyano group],
or its N oxide or agriculturally acceptable salt thereof.

9. The compound according to claim 8 wherein
E represents R¹⁹O—,
R¹⁹ represents a phenyl group which is substituted at its 2-position with substituent(s) selected from Group V, a phenyl group which is substituted at its 4-position with substituent(s) selected from Group V, a 3-chlorophenyl group, a phenyl group which is substituted with two or more halogen atoms, or a pyridyl group which may be optionally substituted with one or more halogen atoms,
Group V is a group consisting of a halogen atom and a methoxy group,
or its N oxide or agriculturally acceptable salt thereof.

10. An agricultural composition which comprises the compound according to claim 8 or its N-oxide compound or an agriculturally acceptable salt thereof and an inert carrier.

11. The agricultural composition according to claim 10 which comprises one or more ingredients selected from the group consisting of the following Groups (a), (b), (c) and (d):
Group (a): a group consisting of insecticidal ingredients, miticidal ingredients, and nematicidal ingredients;
Group (b): fungicidal ingredients:
Group (c): plant growth modulating ingredients; and
Group (d): repellent ingredients.

12. A method for controlling a soybean rust fungus having an amino acid substitution of F129L on mitochondrial cytochrome b protein, which comprises applying an effective amount of a compound represented by formula (III): 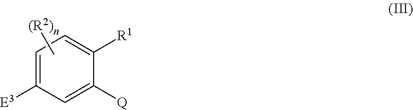
[wherein
E³ represents R³⁹OCH₂—, R⁴⁰CH₂O—, R⁴¹S—, R⁴²SCH₂—, R⁴³CH₂S—, or R⁴⁶O—,
Q represents a group represented by Q1, a group represented by Q2, a group represented by Q3, a group represented by Q4, a group represented by Q5, a group represented by Q6, a group represented by Q7, or a group represented by Q8,
each of the group represented by Q1, the group represented by Q2, the group represented by Q3, the group represented by Q4, the group represented by Q5, the group represented by Q6, the group represented by Q7, and the group represented by Q8 represents the following group respectively (where • represents a binding site to a benzene ring), 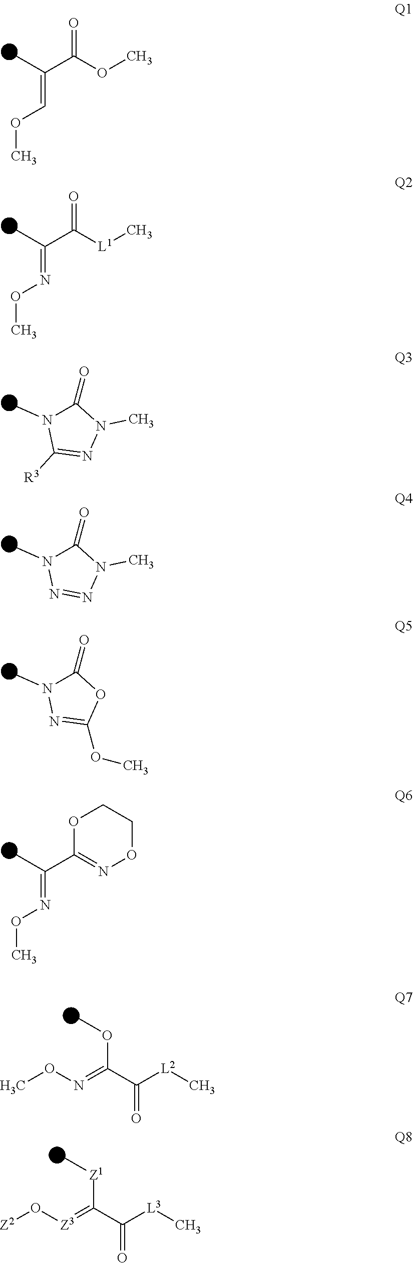
L¹, L² and L³ are identical to or different from each other and represent an oxygen atom, or NH,
Z¹ represents a single bond, CH₂, an oxygen atom or NCH₃,
Z² represents a methyl group which is substituted with one or more fluorine atoms,
Z³ represents CH or a nitrogen atom,
R¹ represents a C1-C3 chain hydrocarbon group, a C1-C3 alkoxy group {the C1-C3 chain hydrocarbon group, and the C1-C3 alkoxy group may be optionally substituted with one or more halogen atoms}, a cyclopropyl group, a halogen atom, or a hydrogen atom,
n is 0, 1, 2, or 3,
when n is 2 or 3, a plural of R² may be identical to or different from each other,
R² represents a C1-C3 chain hydrocarbon group, a C1-C3 alkoxy group {the C1-C3 chain hydrocarbon group, and the C1-C3 alkoxy group may be optionally substituted with one or more halogen atoms}, a cyclopropyl group, or a halogen atom,
R³⁹ represents a five- to ten-membered aromatic heterocyclic group which may be optionally substituted with one or more substituents selected from Group R,
R⁴⁰ represents a C6-C10 aryl group, or a five- to ten-membered aromatic heterocyclic group {the C6-C10 aryl group, and the five- to ten-membered aromatic heterocyclic group may be optionally substituted with one or more substituents selected from Group R},
R⁴¹ represents a C1-C6 chain hydrocarbon group which may be optionally substituted with one or more substituents selected from Group S, a C6-C10 aryl group, a five- to ten-membered aromatic heterocyclic group {the C6-C10 aryl group, and the five- to ten-membered aromatic heterocyclic group may be optionally substituted with one or more substituents selected from Group R}, or a C3-C6 cycloalkyl group which may be optionally substituted with one or more substituents selected from Group T,
R⁴² represents a C6-C10 aryl group, or a five- to ten-membered aromatic heterocyclic group {the C6-C10 aryl group, and the five- to ten-membered aromatic heterocyclic group may be optionally substituted with one or more substituents selected from Group R},
R⁴³ represents a C6-C10 aryl group, or a five- to ten-membered aromatic heterocyclic group {the C6-C10 aryl group, and the five- to ten-membered aromatic heterocyclic group may be optionally substituted with one or more substituents selected from Group R},
R⁴⁶ represents a C5-C6 cycloalkyl group which may be optionally substituted with one or more substituents selected from Group T.
Group R is a group consisting of a C1-C6 chain hydrocarbon group, a C1-C6 alkoxy group, a C1-C6 alkylthio group {the C1-C6 chain hydrocarbon group, the C1-C6 alkoxy group, and the C1-C6 alkylthio group may be optionally substituted with one or more substituents selected from Group S}, a C3-C6 cycloalkyl group which may be optionally substituted with one or more substituents selected from Group T, a phenyl group, a five- to six-membered aromatic heterocyclic group {the phenyl group, and the five- to six-membered aromatic heterocyclic group may be optionally substituted with one or more substituents selected from Group U}, a halogen atom, a cyano group, and a nitro group.
Group S is a group consisting of a C3-C4 cycloalkyl group, a halogen atom, and a C1-C3 alkoxy group.
Group T is a group consisting of a C1-C3 chain hydrocarbon group which may be optionally substituted with one or more halogen atoms, a halogen atom, and a cyano group.
Group U is a group consisting of a C1-C6 chain hydrocarbon group, a C1-C6 alkoxy group, a C1-C6 alkylthio group {the C1-C6 chain hydrocarbon group, the C1-C6 alkoxy group, and the C1-C6 alkylthio group may be optionally substituted with one or more substituents selected from Group S}, a C3-C6 cycloalkyl group which may be optionally substituted with one or more substituents selected from Group T, a halogen atom, a cyano group, and a nitro group.],
or its N oxide or agriculturally acceptable salt thereof.

13. The method according to claim 12 wherein the compound represented by formula (III) is a compound wherein
E³ represents R³⁹OCH₂—, R⁴⁰CH₂O—, R⁴¹S—, R⁴²SCH₂—, or R⁴³CH₂S—,
R⁴¹ represents a C1-C6 chain hydrocarbon group which may be optionally substituted with one or more substituents selected from Group S, a C6-C10 aryl group, or a five- to ten-membered aromatic heterocyclic group {the C6-C10 aryl group, and the five- to ten-membered aromatic heterocyclic group may be optionally substituted with one or more substituents selected from Group R.}.

14. (canceled)

15. A composition for controlling a plant disease, which comprises a compound represented by the following formula (IV) and one or more of compounds selected from Group (B),
a compound represented by formula (IV): 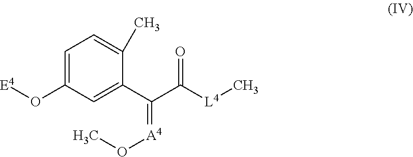
[wherein
E⁴ represents a phenyl group, a 2-methylphenyl group, a 3-methylphenyl group, a 4-methylphenyl group, a 3-fluorophenyl group, a 3-methoxyphenyl group, a 3-(trifluoromethoxy)phenyl group, a 3-phenoxyphenyl group, a 3-thienyl group, a 2-thiazolyl group, a 4-(trifluoromethyl)thizol-2-yl group, a 5-chlorothiazol-2-yl group, a 5-bromothiazol-2-yl group, a 5-(trifluoromethyl)thiazol-2-yl group, a 5-cyclopropylthiazol-2-yl group, a 5-ethynylthiazol-2-yl group, or a (5-cyclopropylethynyl)thiazol-2-yl group,
a combination of A⁴ and L⁴ represents
a combination wherein
A⁴ represents CH, and L⁴ represents an oxygen atom;
or
a combination wherein
A⁴ represents a nitrogen atom, and L⁴ represents NH.],
Group (B) is a group consisting of the following sub-groups (B-1), (B-2), (B-3) and (B-4).
Sub-group (B-1) is a mitochondrial electron transport chain complex III inhibitor, which consists of coumoxystrobin, enoxastrobin, flufenoxystrobin, picoxystrobin, pyraoxystrobin, pyraclostrobin, mandestrobin, pyrametostrobin, triclopyricab, trifloxystrobin, dimoxystrobin, fenaminstrobin, metominostrobin, orysastrobin, fluoxastrobin, fenamidone, pyribencarb, metyltetraprole, cyazofamid, amisulbrom, fenpicoxamid, and florylpicoxamid.
Sub-group (B-2) is a mitochondrial electron transport chain complex II inhibitor, which consists of bixafen, fluxapyroxad, benzovindiflupyr, fluindapyr, pydiflumetofen, 3-(difluoromethyl)-N-(2,3-dihydro-1,1,3-trimethyl-1H-inden-4-yl)-1-methyl-1H-pyrazole-4-carboxamide, a compound represented by the following formula (1), a compound represented by the following formula (2), and a compound represented by the following formula (3). 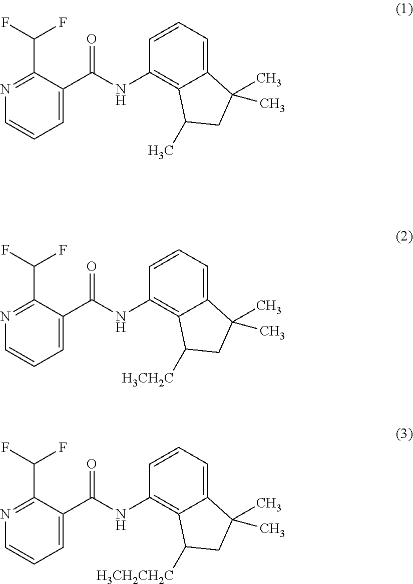
Sub-group (B-3) is a sterol biosynthesis inhibitor, which consists of prothioconazole and mefentrifluconazole.
Sub-group (B-4) is a group consisting of 3-(2-methylphenyl)butan-2-yl N-[(3-acetoxy-4-methoxypyridin-2-yl)carbonyl]alaninate, 3-(4-fluoro-2-methylphenyl)butan-2-yl N-[(3-acetoxy-4-methoxypyridin-2-yl)carbonyl]alaninate, 3-(4-methoxy-2-methylphenyl)butan-2-yl N-[(3-acetoxy-4-methoxypyridin-2-yl)carbonyl]alaninate, 3-(2,4-dimethylphenyl)butan-2-yl N-[(3-acetoxy-4-methoxypyridin-2-yl)carbonyl]alaninate, 3-(2-methylphenyl)butan-2-yl N-({3-[(2-methylpropanoyl)oxy]-4-methoxypyridin-2-yl}carbonyl)alaninate, 3-(4-fluoro-2-methylphenyl)butan-2-yl N-({3-[(2-methylpropanoyl)oxy]-4-methoxypyridin-2-yl}carbonyl)alaninate, 3-(4-methoxy-2-methylphenyl)butan-2-yl N-({3-[(2-methylpropanoyl)oxy]-4-methoxypyridin-2-yl}carbonyl)alaninate, 3-(2,4-dimethylphenyl)butan-2-yl N-({3-[(2-methylpropanoyl)oxy]-4-methoxypyridin-2-yl}carbonyl)alaninate, 3-(2-methylphenyl)butan-2-yl N-[(3-hydroxy-4-methoxypyridin-2-yl)carbonyl]alaninate, 3-(4-fluoro-2-methylphenyl)butan-2-yl N-[(3-hydroxy-4-methoxypyridin-2-yl)carbonyl]alaninate, 3-(4-methoxy-2-methylphenyl)butan-2-yl N-[(3-hydroxy-4-methoxypyridin-2-yl)carbonyl]alaninate, and 3-(2,4-dimethylphenyl)butan-2-yl N-[(3-hydroxy-4-methoxypyridin-2-yl)carbonyl]alaninate.

16. The composition for controlling a plant disease according to claim 15, wherein the compound represented by formula (IV) is a compound wherein
E⁴ represents a phenyl group, a 2-methylphenyl group, a 3-methylphenyl group, a 4-methylphenyl group, a 3-fluorophenyl group, a 3-methoxyphenyl group, a 3-(trifluoromethoxy)phenyl group, or a 3-phenoxyphenyl group, and
Group (B) is a group consisting of the sub-group (B-1), the sub-group (B-2), and the sub-group (B-3).

17. The composition for controlling a plant disease according to claim 16 wherein the compound represented by formula (IV) is
a compound wherein
A⁴ represents CH, L⁴ represents an oxygen atom, E⁴ represents a phenyl group, a 2-methylphenyl group, a 3-methylphenyl group, a 4-methylphenyl group, a 3-fluorophenyl group, a 3-methoxyphenyl group, a 3-(trifluoromethoxy)phenyl group, or a 3-phenoxyphenyl group, or
a compound wherein
A⁴ represents a nitrogen atom, L⁴ represents NH, and E⁴ represents a 2-methylphenyl group, or a 3-fluorophenyl group.

18. The composition for controlling a plant disease according to claim 16 wherein in the Group (B),
the sub-group (B-1) is a group consisting of pyraclostrobin, picoxystrobin, metyltetraprole, fenpicoxamid, and florylpicoxamid, and
the sub-group (B-2) is a group consisting of fluxapyroxad, benzovindiflupyr, fluindapyr, pydiflumetofen, 3-(difluoromethyl)-N-(2,3-dihydro-1,1,3-trimethyl-1H-inden-4-yl)-1-methyl-1H-pyrazole-4-carboxamide, the compound represented by formula (1), the compound represented by formula (2), and the compound represented by formula (3), 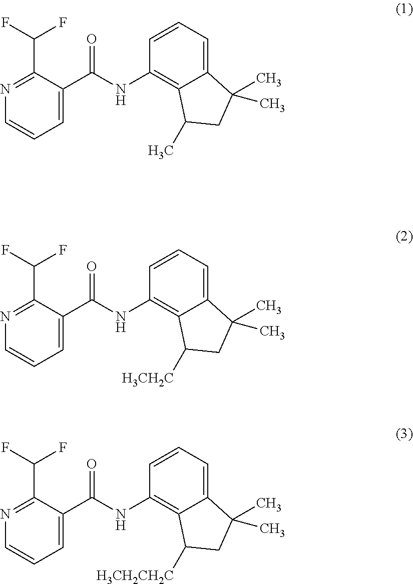
the sub-group (3) is a group consisting of prothioconazole and mefentrifluconazole.

19. The composition for controlling a plant disease according to claim 15 wherein the weight ratio of the compound represented by formula (TV) to one or more compounds selected from Group (B) is within a range of 1:0.01 to 1:100.

20. The composition for controlling a plant disease according to claim 15 wherein a weight ratio of the compound represented by formula (IV) to one of more compounds selected from Group (B) is within a range of 1:0.1 to 1:10.

21. A method for controlling a plant disease which comprises applying an effective amount of the composition for controlling a plant disease according to claim 15 to a plant or soil where a plant is cultivated.

22. (canceled)
